(19) United States
(12) Patent Application Publication
(10) Pub. No.: US 2025/0144094 A1
(43) Pub. Date: May 8, 2025

(54) THIOPHENE ULK1/2 INHIBITORS AND THEIR USE THEREOF

(71) Applicant: Erasca, Inc., San Diego, CA (US)

(72) Inventors: Marcos GONZALEZ-LOPEZ, San Diego, CA (US); Jean-Michel VERNIER, San Diego, CA (US)

(21) Appl. No.: 18/708,947

(22) PCT Filed: Nov. 15, 2022
(86) PCT No.: PCT/US2022/079896

§ 371 Date: May 9, 2024

Related U.S. Application Data
(60) Provisional application No. 63/383,113, filed on Nov. 10, 2022.
(60) Provisional application No. 63/374,491, filed on Sep. 2, 2022.
(60) Provisional application No. 63/338,210, filed on May 4, 2022.
(60) Provisional application No. 63/279,353, filed on Nov. 15, 2021.

(51) Int. Cl. A61K 31/506; A61K 31/554; A61K 31/675; C07D 409/14; C07D 495/04; C07D 513/04; C07D 515/04

(52) U.S. Cl. CPC A61K 31/506; A61K 31/554; A61K 31/675; C07D 409/14; C07D 495/04; C07D 513/04; C07D 515/04

(57) ABSTRACT

Described herein are compounds that are ULK1/2 inhibitors and their use in the treatment of disorders such as cancers.

### CROSS-REFERENCE

This application claims the benefit of U.S. Provisional Application Ser. No. 63/279,353 filed Nov. 15, 2021; U.S. Provisional Application Ser. No. 63/338,210 filed May 4, 2022; U.S. Provisional Application Ser. No. 63/374,491 filed Sep. 2, 2022; and U.S. Provisional Application Ser. No. 63/383,113 filed Nov. 10, 2022; which are hereby incorporated by reference in their entirety.

### FIELD OF THE DISCLOSURE

The disclosure relates to ULK1/2 inhibitors and their use in the treatment of cancer sensitive to ULK1/2 inhibition.

### BACKGROUND

Autophagy, the cell process of self-digestion, plays a role in maintaining energy homoeostasis and protein synthesis and causes degradation of long-lived proteins and damaged organelles, indicating that it plays a role in cancer, by both protecting against and promoting cell death. The autophagy-related gene (Atg) family, with more than 35 members, regulates multiple stages of the process. UNC-51-like kinase 1 (ULK1) has been demonstrated to mediate autophagy. Studies have indicated that inhibition of ULK1 promotes apoptosis and suppresses tumor growth and metastasis in cancers. Dower et al., Mol. Cancer Ther; 17(11), 2018, pp. 2366-2376; Martin et al., iScience; 8, 2018, pp. 74-84; Tompkins et al., Yale Journal of Biology and Medicine; 92, 2019, pp. 707-718; Lin et al., Cell Death and Disease; 10, 2019, p. 139.

There is therefore an urgent need for developing ULK1 inhibitors for the treatment of cancers in subjects, including humans.

### SUMMARY OF THE DISCLOSURE

The present disclosure provides novel ULK1/2 inhibitors, and their use in the treatment of cancers which are sensitive to ULK1/2 inhibition (e.g. CML).

Disclosed herein is a compound of Formula (I), or a pharmaceutically acceptable salt thereof:

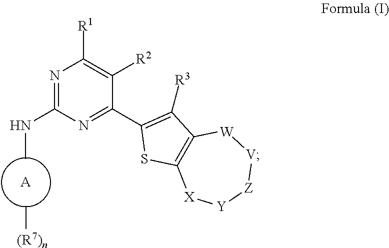

wherein
R¹ is hydrogen, halogen, C₁-C₆alkyl, C₁-C₆haloalkyl, C₁-C₆hydroxyalkyl, C₁-C₆aminoalkyl, or C₁-C₆heteroalkyl;
R² is hydrogen, halogen, C₁-C₆alkyl, C₁-C₆haloalkyl, C₁-C₆hydroxyalkyl, C₁-C₆aminoalkyl, C₁-C₆heteroalkyl, —C(═O)NR^{c}R^{d}, or cycloalkyl;
R³ is hydrogen, halogen, C₁-C₆alkyl, C₁-C₆haloalkyl, C₁-C₆hydroxyalkyl, C₁-C₆aminoalkyl, or C₁-C₆heteroalkyl;
W is —C(═O)—, —S(═O)—, —S(═O)₂—, —C(═O)NR⁴—, —S(═O)₂NR⁴—, —NR⁴S(═O)₂—, or —S(═O)(═NR⁴)—;
X is —NR⁵—, —O—, —S—, —S(═O)₂—, —C(R⁶)₂—, —C(═O)—, —C(═O)NR⁵—, or null;
Y is —C(R⁶)₂—, —O—, —NR⁵—, or null;
Z is —C(R⁶)₂—, —NR⁵—, or null;
or Y—Z is —CR⁶═CR⁶—, —CR⁶═N—, or —N═CR⁶—;
V is —C(R⁶)₂— or null;
wherein

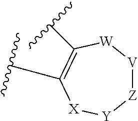

is a 5- to 8-membered ring;
R⁴ is hydrogen or C₁-C₆alkyl;
R⁵ is hydrogen, —C(═O)R^{a}, —C(═O)OR^{b}, —C(═O)NR^{c}R^{d}, —S(═O)₂NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl is optionally and independently substituted with one or more R;
each R⁶ is independently hydrogen, halogen, —CN, —NO₂, —OH, —OR^{a}, —OC(═O)NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R⁶ on the same atom are taken together to form an oxo;
Ring A is aryl or heteroaryl;
each R⁷ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl is optionally and independently substituted with one or more R^{7a};
or two R⁷ on the same atom are taken together to form an oxo;
or two R⁷ are taken together to form a heterocycloalkyl optionally substituted with one or more R^{7b};
each R^{7a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7a} on the same atom are taken together to form an oxo;
each R^{7b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7b} on the same atom are taken together to form an oxo;
n is 0-7;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
or two R on the same atom form an oxo.

Disclosed herein is a compound of Formula (II), or a pharmaceutically acceptable salt thereof:

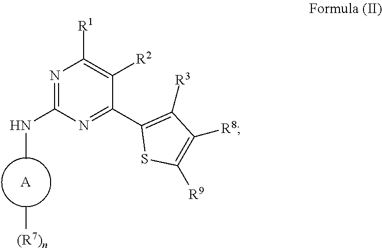

wherein
R¹ is hydrogen, halogen, C₁-C₆alkyl, C₁-C₆haloalkyl, C₁-C₆hydroxyalkyl, C₁-C₆aminoalkyl, or C₁-C₆heteroalkyl;
R² is halogen, C₁-C₆alkyl, C₁-C₆haloalkyl, C₁-C₆hydroxyalkyl, C₁-C₆aminoalkyl, C₁-C₆heteroalkyl, —C(═O)NR^{c}R^{d}, or cycloalkyl;
R³ is hydrogen, halogen, C₁-C₆alkyl, C₁-C₆haloalkyl, C₁-C₆hydroxyalkyl, C₁-C₆aminoalkyl, or C₁-C₆heteroalkyl;
R⁸ is —CN, —NR^{c}R^{d}, —C(═O)NR^{c}R^{d}, —C(═O)R^{a}, —S(═O)R^{a}, —S(═O)₂R^{a}, —S(═O)(═NR^{b})R^{a}, —S(═O)₂NR^{c}R^{d}, —NR^{b}S(═O)₂R^{a}, —P(═O)(R^{a})₂, heterocycloalkyl, or heteroaryl; wherein the heterocycloalkyl and heteroaryl is independently optionally substituted with one or more R;
R⁹ is hydrogen, halogen, —C(═O)NR^{c}R^{d}, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkyl(cycloalkyl), or C₁-C₆alkyl(heterocycloalkyl); wherein the alkyl, cycloalkyl, and heterocycloalkyl is optionally and independently substituted with one or more R;
Ring A is aryl or heteroaryl;
each R⁷ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl is optionally and independently substituted with one or more R^{7a};
or two R⁷ on the same atom are taken together to form an oxo;
or two R⁷ are taken together to form a heterocycloalkyl optionally substituted with one or more R^{7b};
each R^{7a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7a} on the same atom are taken together to form an oxo;
each R^{7b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NRC(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7b} on the same atom are taken together to form an oxo;
n is 0-7;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
or two R on the same atom form an oxo;
provided that the compound is not

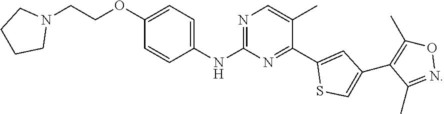 [SMILES: Cc1cnc(Nc2ccc(OCCN3CCCC3)cc2)nc1-c1cc(-c2c(C)noc2C)cs1]

Also disclosed herein is a pharmaceutical composition comprising a compound disclosed herein, or pharmaceutically acceptable salt thereof, and a pharmaceutically acceptable excipient.

Also disclosed herein is a method of inhibiting a Unc-51 like autophagy activating kinase (ULK) isoform comprising contacting the ULK isoform with a compound disclosed herein, or pharmaceutically acceptable salt thereof, or a pharmaceutical composition disclosed herein.

In some embodiments, the method inhibits ULK1 and ULK2.

Also disclosed herein is a method of treating cancer comprising administering to a subject a compound disclosed herein, or pharmaceutically acceptable salt thereof, or pharmaceutical a composition disclosed herein.

Also disclosed herein is a use of a compound disclosed herein, or pharmaceutically acceptable salt thereof, in the manufacture of a medicament for the treatment of cancer.

### DETAILED DESCRIPTION OF THE DISCLOSURE

“Abnormal cell growth”, as used herein, unless otherwise indicated, refers to cell growth that is independent of normal regulatory mechanisms (e.g., loss of contact inhibition). Abnormal cell growth may be benign (not cancerous), or malignant (cancerous). Abnormal cell growth includes the abnormal growth of: (1) tumor cells (tumors) that show increased expression of ULK1 or ULK2; (2) tumors that proliferate by aberrant ULK1 or ULK2 activation; and/or (3) tumors characterized by amplification or overexpression of the genes that express ULK1 or ULK2.

The term “additional anticancer agents” as used herein means any one or more therapeutic agent, other than a compound of the disclosure, that is or can be used in the treatment of cancer. In some embodiments, such additional anticancer agents include compounds derived from the following classes: mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, biological response modifiers, enzyme inhibitors, antisense oligonucleotides or oligonucleotide derivatives, cytotoxics, immuno-oncology agents, and the like.

As used herein “cancer” refers to any malignant and/or invasive growth or tumor caused by abnormal cell growth. Cancer includes solid tumors named for the type of cells that form them, cancer of blood, bone marrow, or the lymphatic system. Examples of solid tumors include sarcomas and carcinomas. Cancers of the blood include, but are not limited to, leukemia, lymphoma, plasmacytoma, extramedullary plasmacytoma, and myeloma.

In some embodiments, the leukemia is acute lymphocytic leukemia (ALL), acute myelogenous leukemia (AML), chronic lymphocytic leukemia (CLL), or chronic myelogenous leukemia (CML).

In some embodiments, the lymphoma is Hodgkin lymphoma, Non-Hodgkin lymphoma, chronic lymphocytic leukemia (CLL), or small lymphocytic lymphoma (SLL).

In some embodiments, the myeloma is multiple myeloma.

Cancer also includes primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type from the latter one.

As used herein, the term “combination therapy” refers to the administration of a compound of the disclosure together with an at least one additional pharmaceutical or medicinal agent (e.g., one or more additional anticancer agents), either sequentially or simultaneously.

As used herein, “subject” refers to a human or animal subject. In certain preferred embodiments, the subject is a human.

The term “treat” or “treating” a cancer as used herein means to administer a compound of the present invention to a subject having cancer, or diagnosed with cancer, to achieve at least one positive therapeutic effect, such as, for example, reduced number of cancer cells, reduced tumor size, reduced rate of cancer cell infiltration into peripheral organs, or reduced rate of tumor metastases or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

As used herein, a “pharmaceutically acceptable carrier” refers to a carrier or diluent that does not cause significant irritation to an organism and does not abrogate the biological activity and properties of the administered compound.

The terms below, as used herein, have the following meanings, unless indicated otherwise:

“Oxo” refers to ═O.

“Carboxyl” refers to —COOH.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, —COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, —COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —OR^{a} where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, —COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aromatic ring, which may be monocyclic or bicyclic (for example, phenyl or naphthyl). In some embodiments, the aryl is a 6-membered aromatic ring (phenyl). Aryl radicals include, but are not limited to anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Aryl radicals include, but are not limited to 1,2,3,5,6,7-hexahydro-s-indacene, 2,3-dihydro-1H-indene, 1,2,3,4-tetrahydronaphthalene, 2,3,5,6,7,8-hexahydro-1H-cyclopenta[b]naphthalene, and 1,2,3,4,5,6,7,8-octahydroanthracene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, —COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom) or bridged ring systems. In some embodiments, the cycloalkyl is fully saturated. Representative cycloalkyls include, but are not limited to, cycloalkyls having from three to fifteen carbon atoms (C₃-C₁₅ cycloalkyl or C₃-C₁₅ cycloalkenyl), from three to ten carbon atoms (C₃-C₁₀ cycloalkyl or C₃-C₁₀ cycloalkenyl), from three to eight carbon atoms (C₃-C₈ cycloalkyl or C₃-C₈ cycloalkenyl), from three to six carbon atoms (C₃-C₆ cycloalkyl or C₃-C₆ cycloalkenyl), from three to five carbon atoms (C₃-C₅ cycloalkyl or C₃-C₅ cycloalkenyl), or three to four carbon atoms (C₃-C₄ cycloalkyl or C₃-C₄ cycloalkenyl). In some embodiments, the cycloalkyl is a 3- to 10-membered cycloalkyl or a 3- to 10-membered cycloalkenyl. In some embodiments, the cycloalkyl is a 3- to 6-membered cycloalkyl or a 3- to 6-membered cycloalkenyl. In some embodiments, the cycloalkyl is a 5- to 6-membered cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, —COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl or C₂-C₁₅ heterocycloalkenyl), from two to ten carbon atoms (C₂-C₁₀ heterocycloalkyl or C₂-C₁₀ heterocycloalkenyl), from two to eight carbon atoms (C₂-C₈ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₆ heterocycloalkenyl), from two to five carbon atoms (C₂-C₅ heterocycloalkyl or C₂-C₅ heterocycloalkenyl), or two to four carbon atoms (C₂-C₄ heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl or a 3- to 8-membered heterocycloalkenyl. In some embodiments, the heterocycloalkyl is a 3- to 7-membered heterocycloalkyl or a 3- to 7-membered heterocycloalkenyl. In some embodiments, the heterocycloalkyl is a 3- to 6-membered heterocycloalkyl or a 3- to 6-membered heterocycloalkenyl. In some embodiments, the heterocycloalkyl is a 4- to 6-membered heterocycloalkyl or a 4- to 6-membered heterocycloalkenyl. In some embodiments, the heterocycloalkyl is a 5- to 6-membered heterocycloalkyl or a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, —COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). In some embodiments, the heteroaryl is 1,2,3,4-tetrahydroisoquinolinyl, isoindolinyl, 2,3,4,5-tetrahydro-1H-benzo[c]azepinyl, or 2,3,4,5-tetrahydro-1H-benzo[d]azepinyl, the heteroaryl is bonded through a phenyl ring atom. Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

### Compounds

Disclosed herein are compounds of Formula (I), or a pharmaceutically acceptable salt thereof:

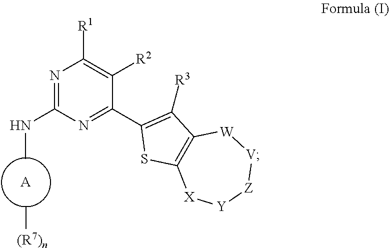

wherein
R¹ is hydrogen, halogen, C₁-C₆alkyl, C₁-C₆haloalkyl, C₁-C₆hydroxyalkyl, C₁-C₆aminoalkyl, or C₁-C₆heteroalkyl;
R² is hydrogen, halogen, C₁-C₆alkyl, C₁-C₆haloalkyl, C₁-C₆hydroxyalkyl, C₁-C₆aminoalkyl, C₁-C₆heteroalkyl, —C(═O)NR^{c}R^{d}, or cycloalkyl;
R³ is hydrogen, halogen, C₁-C₆alkyl, C₁-C₆haloalkyl, C₁-C₆hydroxyalkyl, C₁-C₆aminoalkyl, or C₁-C₆heteroalkyl;
W is —C(═O)—, —S(═O)—, —S(═O)₂—, —C(═O)NR⁴—, —S(═O)₂NR⁴—, —NR⁴S(═O)₂—, or —S(═O)(═NR⁴)—;
X is —NR⁵—, —O—, —S—, —S(═O)₂—, —C(R⁶)₂—, —C(═O)—, —C(═O)NR⁵—, or null;
Y is —C(R⁶)₂—, —O—, —NR⁵—, or null;
Z is —C(R⁶)₂—, —NR—, or null;
or Y—Z is —CR⁶═CR⁶—, —CR⁶═N—, or —N═CR⁶—
V is —C(R⁶)₂— or null;
wherein

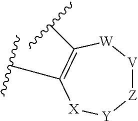

is a 5- to 8-membered ring;
R⁴ is hydrogen or C₁-C₆alkyl;
R⁵ is hydrogen, —C(═O)R^{a}, —C(═O)OR^{b}, —C(═O)NR^{c}R^{d}, —S(═O)₂NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl is optionally and independently substituted with one or more R;
each R⁶ is independently hydrogen, halogen, —CN, —NO₂, —OH, —OR^{a}, —OC(═O)NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R⁶ on the same atom are taken together to form an oxo;
Ring A is aryl or heteroaryl;
each R⁷ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl is optionally and independently substituted with one or more R^{7a};
or two R⁷ on the same atom are taken together to form an oxo;
or two R⁷ are taken together to form a heterocycloalkyl optionally substituted with one or more R^{7b};
each R^{7a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7a} on the same atom are taken together to form an oxo;
each R^{7b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7b} on the same atom are taken together to form an oxo;
n is 0-7;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, C₁-C₆heteroalkyl cycloalkyl, or heterocycloalkyl;
or two R on the same atom form an oxo.

Disclosed herein are compounds of Formula (I), or a pharmaceutically acceptable salt thereof:

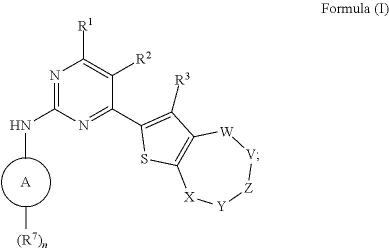

wherein
R¹ is hydrogen, halogen, C₁-C₆alkyl, C₁-C₆haloalkyl, C₁-C₆hydroxyalkyl, C₁-C₆aminoalkyl, or C₁-C₆heteroalkyl;
R² is hydrogen, halogen, C₁-C₆alkyl, C₁-C₆haloalkyl, C₁-C₆hydroxyalkyl, C₁-C₆aminoalkyl, C₁-C₆heteroalkyl, —C(═O)NR^{c}R^{d}, or cycloalkyl;
R³ is hydrogen, halogen, C₁-C₆alkyl, C₁-C₆haloalkyl, C₁-C₆hydroxyalkyl, C₁-C₆aminoalkyl, or C₁-C₆heteroalkyl;
W is —C(═O)—, —S(═O)—, —S(═O)₂—, —C(═O)NR⁴—, —S(═O)₂NR⁴—, —NR⁴S(═O)₂—, or —S(═O)(═NR⁴)—;
X is —NR⁵—, —O—, —S—, —S(═O)₂—, —C(R⁶)₂—, —C(═O)—, —C(═O)NR⁵—, or null;
Y is —C(R⁶)₂—, —NR⁵—, or null;
Z is —C(R⁶)₂—, —NR⁵—, or null;
or Y—Z is —CR⁶═CR⁶—, —CR⁶═N—, or —N═CR⁶—;
V is —C(R⁶)₂— or null;
wherein

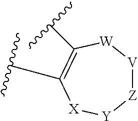

is a 5- to 8-membered ring;
R⁴ is hydrogen or C₁-C₆alkyl;
R⁵ is hydrogen, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl is optionally and independently substituted with one or more R;
each R⁶ is independently hydrogen, halogen, —CN, —NO₂, —OH, —OR^{a}, —OC(═O)NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R⁶ on the same atom are taken together to form an oxo;
Ring A is aryl or heteroaryl;
each R⁷ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{7a};
or two R⁷ on the same atom are taken together to form an oxo;
or two R⁷ are taken together to form a heterocycloalkyl optionally substituted with one or more R^{7b};
each R^{7a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7a} on the same atom are taken together to form an oxo;
each R^{7b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7b} on the same atom are taken together to form an oxo;
n is 0-7;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, and C₁-C₆heteroalkyl;
or two R on the same atom form an oxo.

Disclosed herein are compounds of Formula (I), or a pharmaceutically acceptable salt thereof:

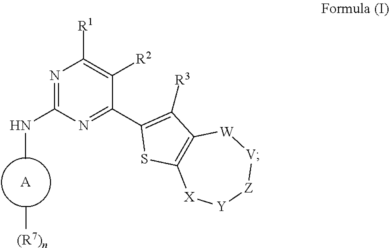

wherein
R¹ is hydrogen, halogen, C₁-C₆alkyl, C₁-C₆haloalkyl, C₁-C₆hydroxyalkyl, C₁-C₆aminoalkyl, or C₁-C₆heteroalkyl;
R² is hydrogen, halogen, C₁-C₆alkyl, C₁-C₆haloalkyl, C₁-C₆hydroxyalkyl, C₁-C₆aminoalkyl, C₁-C₆heteroalkyl, —C(═O)NR^{c}R^{d}, or cycloalkyl;
R³ is hydrogen, halogen, C₁-C₆alkyl, C₁-C₆haloalkyl, C₁-C₆hydroxyalkyl, C₁-C₆aminoalkyl, or C₁-C₆heteroalkyl;
W is —C(═O)—, —S(═O)—, —S(═O)₂—, —C(═O)NR⁴—, —S(═O)₂NR⁴—, —NR⁴S(═O)₂—, or —S(═O)(═NR⁴)—;
X is —NR⁵—, —O—, —S—, —C(R⁶)₂—, —C(═O)—, —C(═O)NR⁵—, or null;
Y is —C(R⁶)₂— or null;
Z is —C(R⁶)₂— or null;
or Y—Z is —CR⁶═CR⁶—, —CR⁶═N—, or —N═CR⁶—;
V is —C(R⁶)₂— or null;
wherein

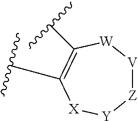

is a 5- to 8-membered ring;
R⁴ is hydrogen or C₁-C₆alkyl;
R⁵ is hydrogen, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
each R⁶ is independently hydrogen, halogen, —CN, —NO₂, —OH, —OR^{a}, —OC(═O)NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R⁶ on the same atom are taken together to form an oxo;
Ring A is aryl or heteroaryl;
each R⁷ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{7a};
or two R⁷ on the same atom are taken together to form an oxo;
or two R⁷ are taken together to form a heterocycloalkyl optionally substituted with one or more R^{7b};
each R^{7a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7a} on the same atom are taken together to form an oxo;
each R^{7b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7b} on the same atom are taken together to form an oxo;
n is 0-7;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, and C₁-C₆heteroalkyl; or two R on the same atom form an oxo.

In some embodiments of a compound of Formula (I), W is —C(═O)NR⁴—. In some embodiments of a compound of Formula (I), W is —S(═O)₂NR⁴— or —NR⁴S(═O)₂—. In some embodiments of a compound of Formula (I), W is —S(═O)₂NR⁴—. In some embodiments of a compound of Formula (I), W is —NR⁴S(═O)₂—. In some embodiments of a compound of Formula (I), W is —C(═O)—. In some embodiments of a compound of Formula (I), W is —S(═O)₂—. In some embodiments of a compound of Formula (I), W is —S(═O)(═NR⁴)—. In some embodiments of a compound of Formula (I), X is —NR⁵—. In some embodiments of a compound of Formula (I), X is —O—. In some embodiments of a compound of Formula (I), X is —S—. In some embodiments of a compound of Formula (I), X is —C(═O)—. In some embodiments of a compound of Formula (I), X is —C(═O)NR⁵—. In some embodiments of a compound of Formula (I), X is —C(R⁶)₂—. In some embodiments of a compound of Formula (I), X is null. In some embodiments of a compound of Formula (I), Y is —C(R⁶)₂—. In some embodiments of a compound of Formula (I), Y is —NR⁵—. In some embodiments of a compound of Formula (I), Y is —O—. In some embodiments of a compound of Formula (I), Y is null. In some embodiments of a compound of Formula (I), Z is —C(R⁶)₂—. In some embodiments of a compound of Formula (I), Z is null. In some embodiments of a compound of Formula (I), Y—Z is —CR═CR⁶—. In some embodiments of a compound of Formula (I), Y—Z is —CR⁶═N—. In some embodiments of a compound of Formula (I), Y—Z is —N═CR⁶—. In some embodiments of a compound of Formula (I), V is —C(R⁶)₂—. In some embodiments of a compound of Formula (I), V is null.

In some embodiments of a compound of Formula (I),

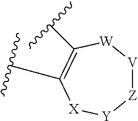

is a 5- to 7-membered ring. In some embodiments of a compound of Formula (I),

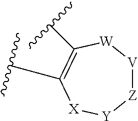

is a 5- to 6-membered ring. In some embodiments of a compound of Formula (I),

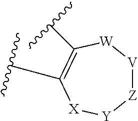

is a 5-membered ring. In some embodiments of a compound of Formula (I),

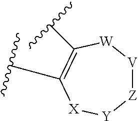

is a 6-membered ring. In some embodiments of a compound of Formula (I),

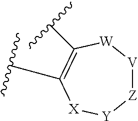

is a 7-membered ring. In some embodiments of a compound of Formula (I),

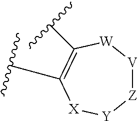

is a 8-membered ring.

In some embodiments of a compound of Formula (I), W is —C(═O)NR⁴—; V is null; Z is null, Y is null, and X is —C(R⁶)₂—. In some embodiments of a compound of Formula (I), W is —C(═O)NR⁴—; V is null; Z is null, Y is —C(R⁶)₂—, and X is —C(R⁶)₂—. In some embodiments of a compound of Formula (I), W is —C(═O)NR⁴—; V is null; Z is —C(R⁶)₂—, Y is —C(R⁶)₂—, and X is —C(R⁶)₂—. In some embodiments of a compound of Formula (I), W is —C(═O)NR⁴—; V is null; Z is —C(R⁶)₂—, Y is —C(R⁶)₂—, and X is —O—. In some embodiments of a compound of Formula (I), W is —C(═O)NR⁴—; V is null; Z is —C(R⁶)₂—, Y is —C(R⁶)₂—, and X is —NR⁵—. In some embodiments of a compound of Formula (I), W is —C(═O)NR⁴—; V is null; Y—Z is —CR⁶═CR⁶—, and X is null. In some embodiments of a compound of Formula (I), W is —C(═O)NR⁴—; V is null; Y—Z is —CR⁶═N—, and X is null. In some embodiments of a compound of Formula (I), W is —C(═O)NR⁴—; V is null; Y—Z is —N═CR⁶—, and X is null.

In some embodiments of a compound of Formula (I), W is —S(═O)₂NR⁴—; V is null; Z is null, Y is null, and X is —C(R⁶)₂—. In some embodiments of a compound of Formula (I), W is —S(═O)₂NR⁴—; V is null; Z is null, Y is —C(R⁶)₂—, and X is —C(R⁶)₂—. In some embodiments of a compound of Formula (I), W is —S(═O)₂NR⁴—; V is null; Z is —C(R⁶)₂—, Y is —C(R⁶)₂—, and X is —C(R⁶)₂—. In some embodiments of a compound of Formula (I), W is —S(═O)₂NR⁴—; V is null; Z is —C(R⁶)₂—, Y is —C(R⁶)₂—, and X is —O—. In some embodiments of a compound of Formula (I), W is —S(═O)₂NR⁴—; V is null; Z is —C(R⁶)₂—, Y is —C(R⁶)₂—, and X is —NR₅—. In some embodiments of a compound of Formula (I), W is —S(═O)₂NR⁴—; V is null; Y—Z is —CR⁶═N—, and X is null. In some embodiments of a compound of Formula (I), W is —S(═O)₂NR⁴—; V is null; Y—Z is —N═CR⁶—, and X is null.

In some embodiments of a compound of Formula (I), W is —NR⁴S(═O)₂—; V is null; Z is null, Y is null, and X is —C(R⁶)₂—. In some embodiments of a compound of Formula (I), W is —NR⁴S(═O)₂—; V is null; Z is null, Y is —C(R⁶)₂—, and X is —C(R⁶)₂—. In some embodiments of a compound of Formula (I), W is —NR⁴S(═O)₂—; V is null; Z is —C(R⁶)₂—, Y is —C(R⁶)₂—, and X is —C(R⁶)₂—. In some embodiments of a compound of Formula (I), W is —NR⁴S(═O)₂—; V is null; Z is —C(R⁶)₂—, Y is —C(R⁶)₂—, and X is —O—. In some embodiments of a compound of Formula (I), W is —NR⁴S(═O)₂—; V is null; Z is —C(R⁶)₂—, Y is —C(R⁶)₂—, and X is —NR⁵—. In some embodiments of a compound of Formula (I), W is —NR⁴S(═O)₂—; V is null; Y—Z is —CR⁶═N—, and X is null. In some embodiments of a compound of Formula (I), W is —NR⁴S(═O)₂—; V is null; Y—Z is —N═CR⁶—, and X is null.

In some embodiments of a compound of Formula (I), W is —C(═O)NR⁴—; V is null; Z is null, Y is null, and X is —C(═O)NR⁵—. In some embodiments of a compound of Formula (I), W is —C(═O)—; V is null; Z is —C(R⁶)₂—, Y is —C(R⁶)₂—, and X is —C(═O)NR⁵—. In some embodiments of a compound of Formula (I), W is —C(═O)NR⁴—; V is null; Z is —C(R⁶)₂—, Y is null, and X is —NR⁵—. In some embodiments of a compound of Formula (I), W is —C(═O)NR⁴—; V is null; Z is —C(R⁶)₂—, Y is —C(R⁶)₂—, and X is —C(═O)—.

In some embodiments of a compound of Formula (I),

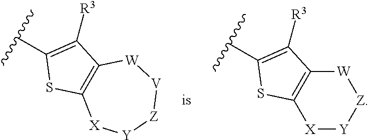

In some embodiments of a compound of Formula (I),

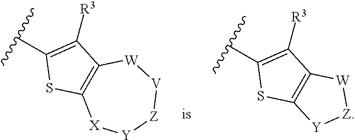

In some embodiments of a compound of Formula (I),

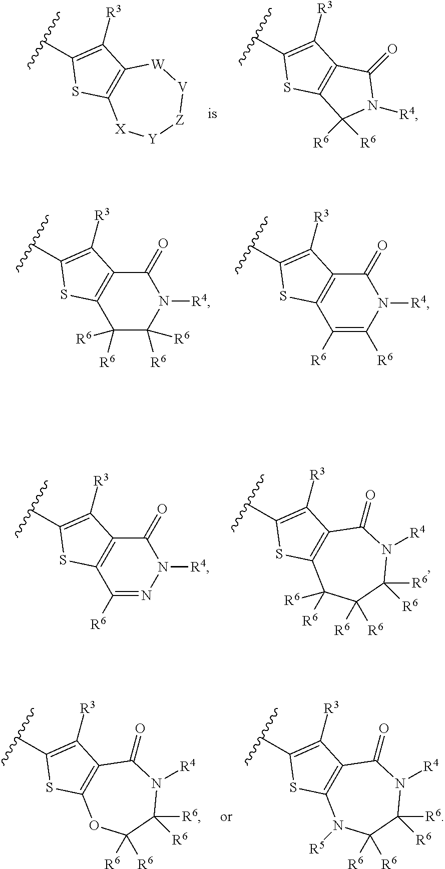

In some embodiments of a compound of Formula (I),

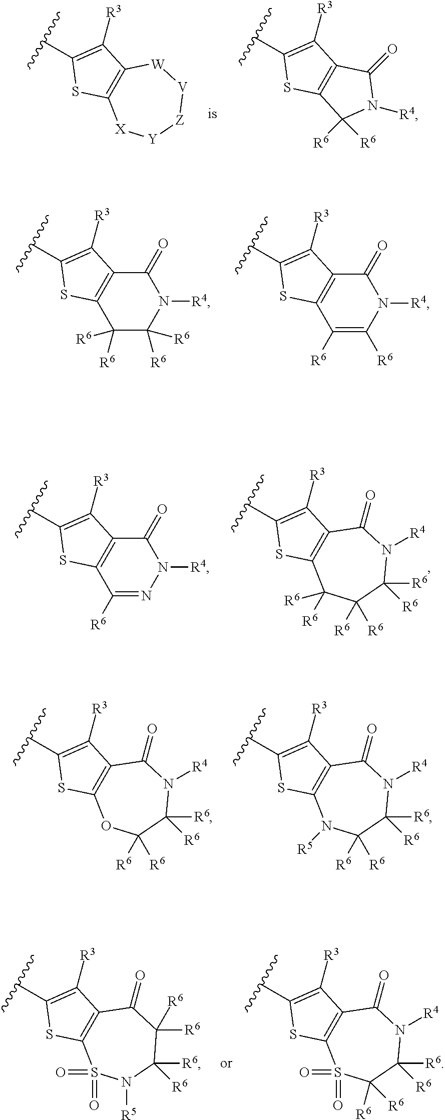

In some embodiments of a compound of Formula (I),

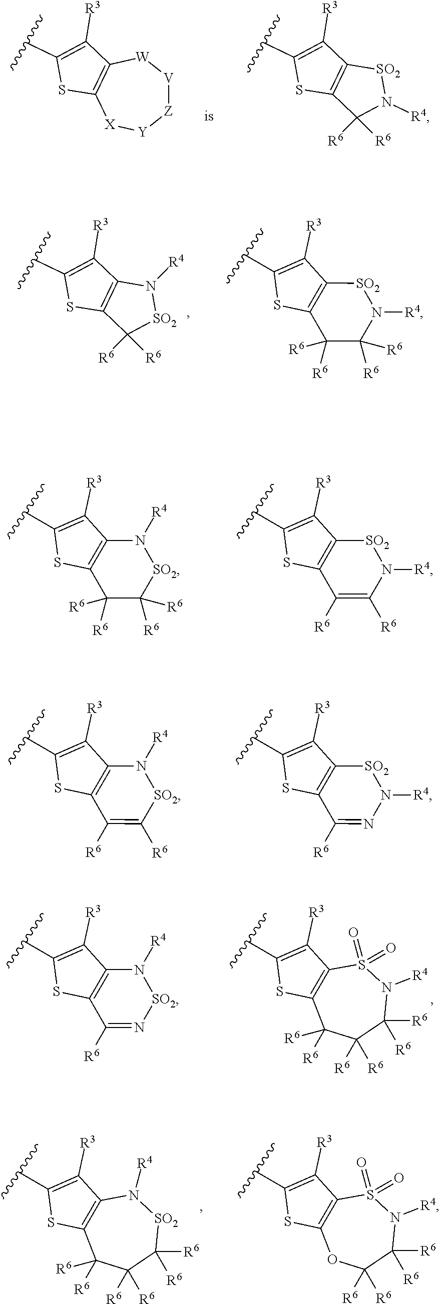

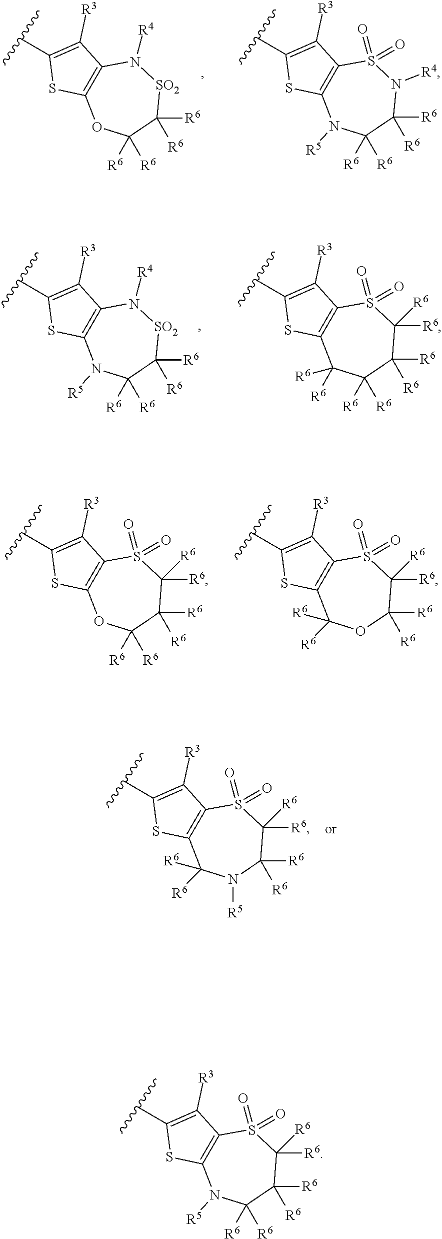

In some embodiments of a compound of Formula (I),

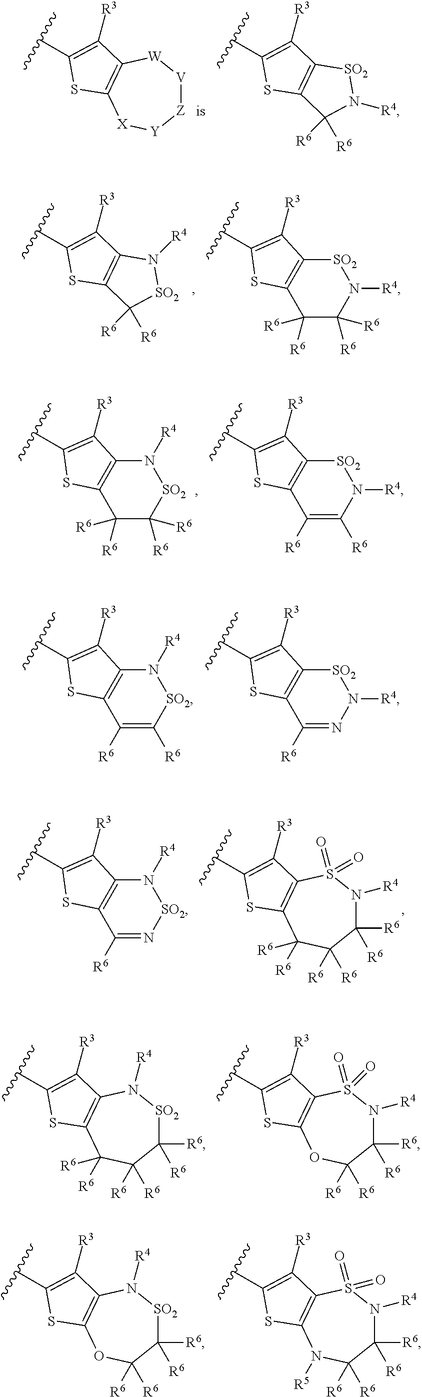

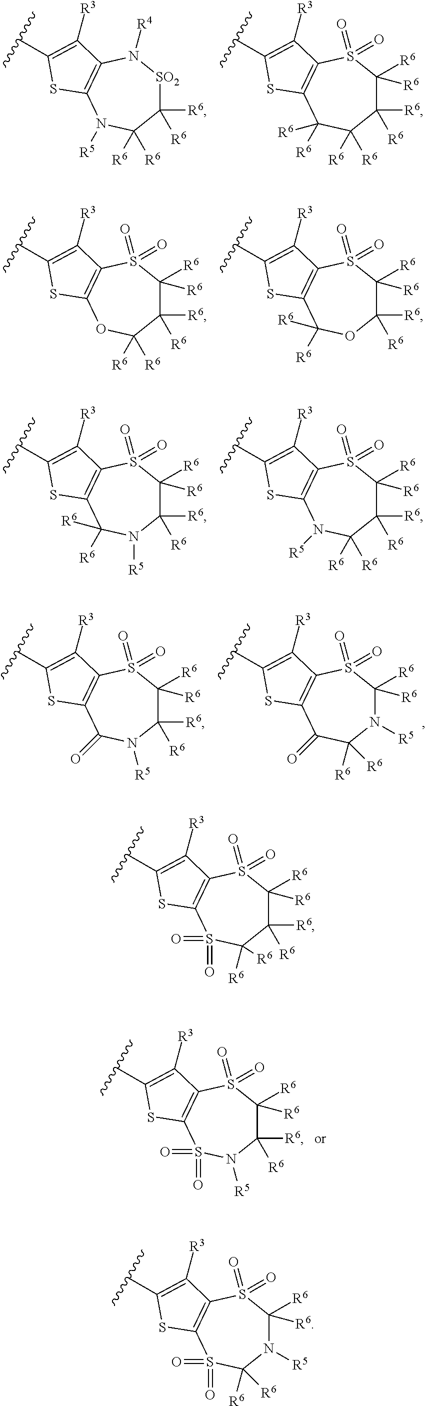

In some embodiments of a compound of Formula (I),

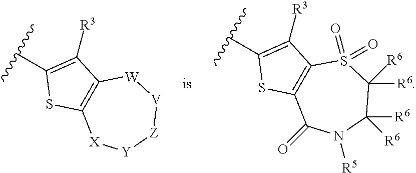

In some embodiments of a compound of Formula (I),

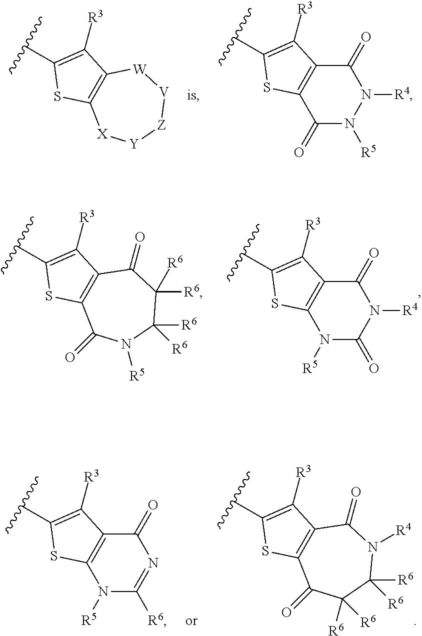

In some embodiments of a compound of Formula (I), R⁴ is hydrogen. In some embodiments of a compound of Formula (I), R⁴ is C₁-C₆alkyl.

In some embodiments of a compound of Formula (I), R⁵ is hydrogen, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkyl(cycloalkyl), or C₁-C₆alkyl(heterocycloalkyl); wherein the alkyl, cycloalkyl, and heterocycloalkyl is optionally and independently substituted with one or more R.

In some embodiments of a compound of Formula (I), R⁵ is hydrogen, C₁-C₆alkyl, C₁-C₆haloalkyl, cycloalkyl, heterocycloalkyl, C₁-C₆alkyl(cycloalkyl), or C₁-C₆alkyl(heterocycloalkyl); wherein the alkyl, cycloalkyl, and heterocycloalkyl is optionally and independently substituted with one or more R.

In some embodiments of a compound of Formula (I), R⁵ is hydrogen, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, or heterocycloalkyl is optionally and independently substituted with one or more R. In some embodiments of a compound of Formula (I), R⁵ is hydrogen, C₁-C₆alkyl, or C₁-C₆haloalkyl; wherein the alkyl is optionally and independently substituted with one or more R. In some embodiments of a compound of Formula (I), R⁵ is hydrogen, C₁-C₆alkyl, or C₁-C₆haloalkyl. In some embodiments of a compound of Formula (I), R⁵ is hydrogen. In some embodiments of a compound of Formula (I), R⁵ is C₁-C₆alkyl.

In some embodiments of a compound of Formula (I), each R⁶ is independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally and independently substituted with one or more R; or two R⁶ on the same atom are taken together to form an oxo. In some embodiments of a compound of Formula (I), each R⁶ is independently hydrogen, halogen, C₁-C₆alkyl, or C₁-C₆haloalkyl; or two R⁶ on the same atom are taken together to form an oxo. In some embodiments of a compound of Formula (I), R⁶ is independently hydrogen or C₁-C₆alkyl; or two R⁶ on the same atom are taken together to form an oxo. In some embodiments of a compound of Formula (I), R⁶ is independently hydrogen or C₁-C₆alkyl. In some embodiments of a compound of Formula (I), each R⁶ is hydrogen.

Also disclosed herein is a compound of Formula (II), or pharmaceutically acceptable salt thereof:

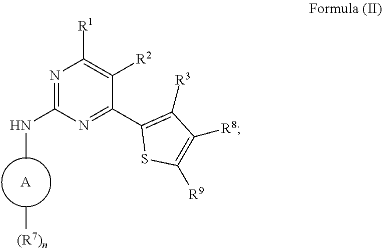

wherein
R¹ is hydrogen, halogen, C₁-C₆alkyl, C₁-C₆haloalkyl, C₁-C₆hydroxyalkyl, C₁-C₆aminoalkyl, or C₁-C₆heteroalkyl;
R² is halogen, C₁-C₆alkyl, C₁-C₆haloalkyl, C₁-C₆hydroxyalkyl, C₁-C₆aminoalkyl, C₁-C₆heteroalkyl, —C(═O)NR^{c}R^{d}, or cycloalkyl;
R³ is hydrogen, halogen, C₁-C₆alkyl, C₁-C₆haloalkyl, C₁-C₆hydroxyalkyl, C₁-C₆aminoalkyl, or C₁-C₆heteroalkyl;
R⁸ is —CN, —NR^{c}R^{d}, —C(═O)NR^{c}R^{d}, —C(═O)R^{a}, —S(═O)R^{a}, —S(═O)₂R^{a}, —S(═O)(═NR^{b})R^{a}, —S(═O)₂NR^{c}R^{d}, —NR^{b}S(═O)₂R^{a}, —P(═O)(R^{a})₂, heterocycloalkyl, or heteroaryl; wherein the heterocycloalkyl and heteroaryl is independently optionally substituted with one or more R;
R⁹ is hydrogen, halogen, —C(═O)NR^{c}R^{d}, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkyl(cycloalkyl), or C₁-C₆alkyl(heterocycloalkyl); wherein the alkyl, cycloalkyl, and heterocycloalkyl is optionally and independently substituted with one or more R;
Ring A is aryl or heteroaryl;
each R⁷ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl is optionally and independently substituted with one or more R^{7a};
or two R⁷ on the same atom are taken together to form an oxo;
or two R⁷ are taken together to form a heterocycloalkyl optionally substituted with one or more R^{7b};
each R^{7a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7a} on the same atom are taken together to form an oxo;
each R^{7b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7b} on the same atom are taken together to form an oxo;
n is 0-7;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
or two R on the same atom form an oxo;
provided that the compound is not

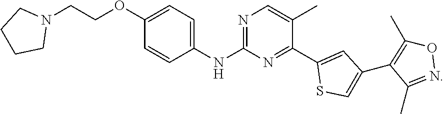 [SMILES: Cc1cnc(Nc2ccc(OCCN3CCCC3)cc2)nc1-c1cc(-c2c(C)noc2C)cs1]

Also disclosed herein is a compound of Formula (II), or pharmaceutically acceptable salt thereof:

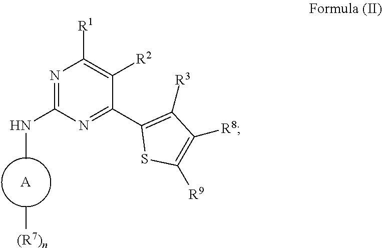

wherein
R¹ is hydrogen, halogen, C₁-C₆alkyl, C₁-C₆haloalkyl, C₁-C₆hydroxyalkyl, C₁-C₆aminoalkyl, or C₁-C₆heteroalkyl;
R² is halogen, C₁-C₆alkyl, C₁-C₆haloalkyl, C₁-C₆hydroxyalkyl, C₁-C₆aminoalkyl, C₁-C₆heteroalkyl, —C(═O)NR^{c}R^{d}, or cycloalkyl;
R³ is hydrogen, halogen, C₁-C₆alkyl, C₁-C₆haloalkyl, C₁-C₆hydroxyalkyl, C₁-C₆aminoalkyl, or C₁-C₆heteroalkyl;
R⁸ is —CN, —NR^{c}R^{d}, —C(═O)NR^{c}R^{d}, —C(═O)R^{a}, —S(═O)R^{a}, —S(═O)₂R^{a}, —S(═O)(═NR^{b})R^{a}, —S(═O)₂NR^{c}R^{d}, —NR^{b}S(═O)₂R^{a}, —P(═O)(R^{a})₂, heterocycloalkyl, or heteroaryl; wherein the heterocycloalkyl and heteroaryl is independently optionally substituted with one or more R; R⁹ is hydrogen, halogen, —C(═O)NR^{c}R^{d}, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkyl(cycloalkyl), or C₁-C₆alkyl(heterocycloalkyl);
Ring A is aryl or heteroaryl;
each R⁷ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{7a};
or two R⁷ on the same atom are taken together to form an oxo;
or two R⁷ are taken together to form a heterocycloalkyl optionally substituted with one or more R^{7b};
each R^{7a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7a} on the same atom are taken together to form an oxo;
each R^{7b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7b} on the same atom are taken together to form an oxo;
n is 0-7;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, and C₁-C₆heteroalkyl; or two R on the same atom form an oxo;
provided that the compound is not

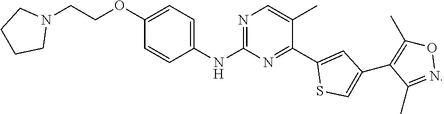 [SMILES: Cc1cnc(Nc2ccc(OCCN3CCCC3)cc2)nc1-c1cc(-c2c(C)noc2C)cs1]

Also disclosed herein is a compound of Formula (II), or pharmaceutically acceptable salt thereof:

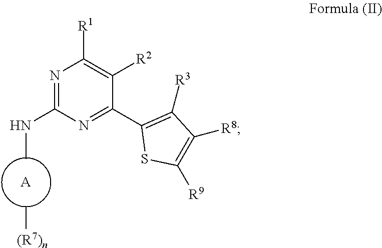

wherein
R¹ is hydrogen, halogen, C₁-C₆alkyl, C₁-C₆haloalkyl, C₁-C₆hydroxyalkyl, C₁-C₆aminoalkyl, or C₁-C₆heteroalkyl;
R² is halogen, C₁-C₆alkyl, C₁-C₆haloalkyl, C₁-C₆hydroxyalkyl, C₁-C₆aminoalkyl, C₁-C₆heteroalkyl, —C(═O)NR^{c}R^{d}, or cycloalkyl;
R³ is hydrogen, halogen, C₁-C₆alkyl, C₁-C₆haloalkyl, C₁-C₆hydroxyalkyl, C₁-C₆aminoalkyl, or C₁-C₆heteroalkyl;
R⁸ is —CN, —NR^{c}R^{d}, —C(═O)NR^{c}R^{d}, —C(═O)R^{a}, —S(═O)R^{a}, —S(═O)₂R^{a}, —S(═O)(═NR^{b})R^{a}, —S(═O)₂NR^{c}R^{d}, —NR^{b}S(═O)₂R^{a}, —P(═O)(R^{a})₂, heterocycloalkyl, or heteroaryl; wherein the heterocycloalkyl and heteroaryl is independently optionally substituted with one or more R;
R⁹ is hydrogen, halogen, —C(═O)NR^{c}R^{d}, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
Ring A is aryl or heteroaryl;
each R⁷ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{7a};
or two R⁷ on the same atom are taken together to form an oxo;
or two R⁷ are taken together to form a heterocycloalkyl optionally substituted with one or more R^{7b};
each R^{7a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7a} on the same atom are taken together to form an oxo;
each R^{7b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7b} on the same atom are taken together to form an oxo;
n is 0-7;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, and C₁-C₆heteroalkyl; or two R on the same atom form an oxo;
provided that the compound is not

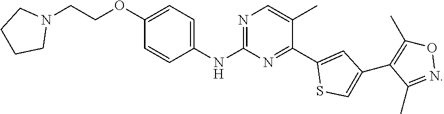 [SMILES: Cc1cnc(Nc2ccc(OCCN3CCCC3)cc2)nc1-c1cc(-c2c(C)noc2C)cs1]

In some embodiments of a compound of Formula (II), R⁸ is —CN, —NR^{c}R^{d}, —C(═O)NR^{c}R^{d}, —S(═O)₂R^{a}, —S(═O)₂NR^{c}R^{d}, —NR^{b}S(═O)₂R^{a}, or —P(═O)(R^{a})₂. In some embodiments of a compound of Formula (II), R⁸ is —C(═O)NR^{c}R^{d}, —S(═O)₂R^{a}, —S(═O)₂NR^{c}R^{d}, —NR^{b}S(═O)₂R^{a}, or —P(═O)(R^{a})₂. In some embodiments of a compound of Formula (II), R⁸ is —C(═O)NR^{c}R^{d}. In some embodiments of a compound of Formula (II), R is —P(═O)(R^{a})₂. In some embodiments of a compound of Formula (II), R⁸ is —C(═O)R^{a}. The compound of claim 36, or pharmaceutically acceptable salt thereof, wherein R⁸ is —S(═O)R^{a}. In some embodiments of a compound of Formula (II), R⁸ is —S(═O)(═NR^{b})R^{a}. In some embodiments of a compound of Formula (II), R⁸ is heterocycloalkyl or heteroaryl; wherein the heterocycloalkyl and heteroaryl is independently optionally substituted with one or more R. In some embodiments of a compound of Formula (II), R⁸ is heterocycloalkyl optionally substituted with one or more R.

In some embodiments of a compound of Formula (II), R⁹ is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (II), R⁹ is hydrogen, halogen, C₁-C₆alkyl, or C₁-C₆haloalkyl.

In some embodiments of a compound of Formula (II), R⁹ is hydrogen or C₁-C₆alkyl. In some embodiments of a compound of Formula (II), R⁹ is hydrogen. In some embodiments of a compound of Formula (II), R⁹ is hydrogen, halogen, —C(═O)NR^{c}R^{d}, C₁-C₆alkyl, or C₁-C₆haloalkyl. In some embodiments of a compound of Formula (II), R⁹ is —C(═O)NR^{c}R^{d}. In some embodiments of a compound of Formula (II), R⁹ is —OR^{a}. In some embodiments of a compound of Formula (II), R⁹ is —NR^{c}R^{d}.

In some embodiments of a compound of Formula (I) or (II), R¹ is hydrogen, halogen, C₁-C₆alkyl, or C₁-C₆haloalkyl. In some embodiments of a compound of Formula (I) or (II), R¹ is hydrogen or C₁-C₂alkyl. In some embodiments of a compound of Formula (I) or (II), R¹ is hydrogen. In some embodiments of a compound of Formula (I) or (II), R¹ is C₁-C₄alkyl. In some embodiments of a compound of Formula (I) or (II), R¹ is C₁-C₃alkyl. In some embodiments of a compound of Formula (I) or (II), R¹ is C₁-C₂alkyl. In some embodiments of a compound of Formula (I) or (II), R¹ is CH₃.

In some embodiments of a compound of Formula (I) or (II), R² is hydrogen, halogen, C₁-C₆alkyl, C₁-C₆haloalkyl, —C(═O)NR^{c}R^{d}, or cycloalkyl. In some embodiments of a compound of Formula (I) or (II), R² is C₁-C₆alkyl, C₁-C₆haloalkyl, —C(═O)NR^{c}R^{d}, or cycloalkyl. In some embodiments of a compound of Formula (I) or (II), R² is C₁-C₂alkyl, C₁-C₂haloalkyl, —C(═O)NR^{c}R^{d}, or cycloalkyl. In some embodiments of a compound of Formula (I) or (II), R² is CF₃. In some embodiments of a compound of Formula (I) or (II), R² is C₁-C₄alkyl. In some embodiments of a compound of Formula (I) or (II), R² is C₁-C₃alkyl. In some embodiments of a compound of Formula (I) or (II), R² is C₁-C₂alkyl. In some embodiments of a compound of Formula (I) or (II), R² is CH₃. In some embodiments of a compound of Formula (I) or (II), R² is cyclopropyl. In some embodiments of a compound of Formula (I) or (II), R² is —C(═O)NH₂.

In some embodiments of a compound of Formula (I) or (II), R³ is hydrogen, halogen, C₁-C₆alkyl, or C₁-C₆haloalkyl. In some embodiments of a compound of Formula (I) or (II), R³ is hydrogen.

In some embodiments of a compound of Formula (I) or (II), Ring A is heteroaryl. In some embodiments of a compound of Formula (I) or (II), Ring A is 5- or 6-membered heteroaryl. In some embodiments of a compound of Formula (I) or (II), Ring A is 5-membered heteroaryl. In some embodiments of a compound of Formula (I) or (II), Ring A is pyrazolyl. In some embodiments of a compound of Formula (I) or (II), Ring A is phenyl.

In some embodiments of a compound of Formula (I) or (II), each R⁷ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkyl(cycloalkyl), or C₁-C₆alkyl(heterocycloalkyl); wherein the alkyl, cycloalkyl, heterocycloalkyl, aryl, and heteroaryl is optionally and independently substituted with one or more R^{7a}; or two R⁷ are taken together to form a heterocycloalkyl optionally substituted with one or more R^{7b}. In some embodiments of a compound of Formula (I) or (II), each R⁷ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkyl(cycloalkyl), or C₁-C₆alkyl(heterocycloalkyl); wherein the alkyl, cycloalkyl, and heterocycloalkyl is optionally and independently substituted with one or more R^{7a}; or two R⁷ are taken together to form a heterocycloalkyl optionally substituted with one or more R^{7b}.

In some embodiments of a compound of Formula (I) or (II), each R⁷ is independently halogen, —CN, —OH, —OR^{a}, —SR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally and independently substituted with one or more R^{7a}; or two R⁷ are taken together to form a heterocycloalkyl optionally substituted with one or more R^{7b}. In some embodiments of a compound of Formula (I) or (II), each R⁷ is independently halogen, —CN, —OH, —OR^{a}, —SR^{a}, —NR^{c}R^{d}, C₁-C₆alkyl, C₁-C₆haloalkyl, cycloalkyl, or heterocycloalkyl; wherein the alkyl, cycloalkyl, and heterocycloalkyl is optionally and independently substituted with one or more R^{7a}; or two R⁷ are taken together to form a heterocycloalkyl optionally substituted with one or more R^{7b}. In some embodiments of a compound of Formula (I) or (II), each R⁷ is independently halogen, —OR^{a}, —SR^{a}, C₁-C₆alkyl, cycloalkyl, or heterocycloalkyl; wherein the alkyl, cycloalkyl, and heterocycloalkyl is optionally and independently substituted with one or more R^{7a}; or two R⁷ are taken together to form a heterocycloalkyl optionally substituted with one or more R^{7b}.

In some embodiments of a compound of Formula (I) or (II), each R^{7a} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally and independently substituted with one or more R. In some embodiments of a compound of Formula (I) or (II), each R^{7a} is independently halogen, C₁-C₆alkyl, or C₁-C₆haloalkyl. In some embodiments of a compound of Formula (I) or (II), each R^{7a} is independently C₁-C₆alkyl.

In some embodiments of a compound of Formula (I) or (II), each R^{7b} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl, is optionally and independently substituted with one or more R. In some embodiments of a compound of Formula (I) or (II), each R^{7b} is independently halogen, C₁-C₆alkyl, C₁-C₆haloalkyl, cycloalkyl, or heterocycloalkyl.

In some embodiments of a compound of Formula (I) or (II), n is 1-4. In some embodiments of a compound of Formula (I) or (II), n is 1-3. In some embodiments of a compound of Formula (I) or (II), n is 1 or 2. In some embodiments of a compound of Formula (I) or (II), n is 1. In some embodiments of a compound of Formula (I) or (II), n is 2. In some embodiments of a compound of Formula (I) or (II), n is 3. In some embodiments of a compound of Formula (I) or (II), n is 4.

In some embodiments of a compound of Formula (I) or (II),

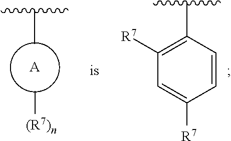

wherein each R⁷ is independently halogen, —OR^{a}, —SR^{a}, C₁-C₆alkyl, cycloalkyl, or heterocycloalkyl; wherein the cycloalkyl and heterocycloalkyl is optionally and independently substituted with one or more C₁-C₆alkyl.

In some embodiments of a compound of Formula (I) or (II),

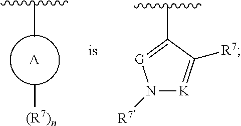

wherein:
G is —CH— and K is —N— or G is —N— and K is —CH—;
R^{7′} is cycloalkyl or heterocycloalkyl; wherein the cycloalkyl and heterocycloalkyl is optionally and independently substituted with one or more C₁-C₆alkyl; and
R⁷ is independently C₁-C₆alkyl, cycloalkyl, or heterocycloalkyl.

In some embodiments of a compound of Formula (I) or (II),

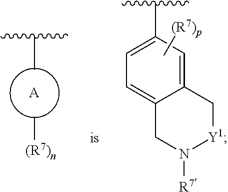

wherein:
Y¹ is —CH₂—, —C(═O)—, or null;
R^{7′} is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{7a};
p is 0-5; and
each R⁷ is independently halogen, —OR^{a}, —SR^{a}, C₁-C₆alkyl, cycloalkyl, or heterocycloalkyl; or two R⁷ on the same atom are taken together to form an oxo.

In some embodiments of a compound of Formula (I) or (II),

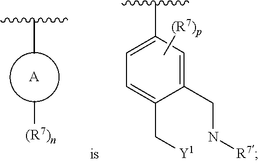

wherein:
Y¹ is —CH₂—, —C(═O)—, or null;
R^{7′} is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{7a};
p is 0-5; and
each R⁷ is independently halogen, —OR^{a}, —SR^{a}, C₁-C₆alkyl, cycloalkyl, or heterocycloalkyl; or two R⁷ on the same atom are taken together to form an oxo.

In some embodiments of a compound of Formula (I) or (II),

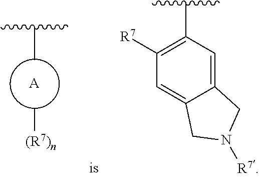

wherein:
R^{7′} is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{7a}; and
R⁷ is independently halogen, —OR^{a}, —SR^{a}, C₁-C₆alkyl, cycloalkyl, or heterocycloalkyl.

In some embodiments of a compound of Formula (I) or (II),

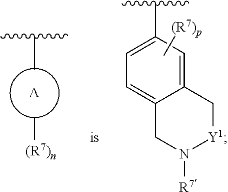

wherein:
Y¹ is —CH₂—, —C(═O)—, or null;
R^{7′} is hydrogen or C₁-C₆alkyl;
p is 0-5; and
each R⁷ is independently halogen, —OR^{a}, —SR^{a}, C₁-C₆alkyl, cycloalkyl, or heterocycloalkyl; or two R⁷ on the same atom are taken together to form an oxo.

In some embodiments of a compound of Formula (I) or (II),

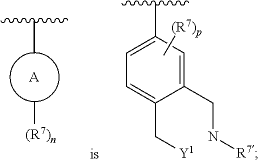

wherein:
Y¹ is —CH₂—, —C(═O)—, or null;
R^{7′} is hydrogen or C₁-C₆alkyl;
p is 0-5; and
each R⁷ is independently halogen, —OR^{a}, —SR^{a}, C₁-C₆alkyl, cycloalkyl, or heterocycloalkyl; or two R⁷ on the same atom are taken together to form an oxo.

In some embodiments of a compound of Formula (I) or (II),

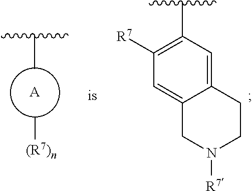

wherein:
R^{7′} is hydrogen or C₁-C₆alkyl; and
R⁷ is independently halogen, —OR^{a}, —SR^{a}, C₁-C₆alkyl, cycloalkyl, or heterocycloalkyl.

In some embodiments of a compound of Formula (I) or (II),

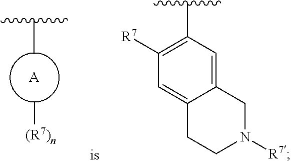

wherein:
R^{7′} is hydrogen or C₁-C₆alkyl; and
R⁷ is independently halogen, —OR^{a}, —SR^{a}, C₁-C₆alkyl, cycloalkyl, or heterocycloalkyl.

In some embodiments of a compound of Formula (I) or (II),

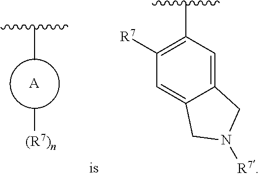

wherein:
R^{7′} is hydrogen or C₁-C₆alkyl; and
R⁷ is independently halogen, —OR^{a}, —SR^{a}, C₁-C₆alkyl, cycloalkyl, or heterocycloalkyl.

In some embodiments of a compound of Formula (I) or (II),

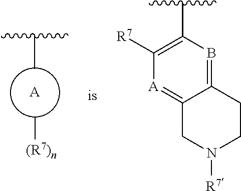

wherein:
A and B are independently selected from CH, N, and CF, with the proviso that at least one of A or B is N or CF.
R^{7′} is hydrogen or C₁-C₆alkyl; and
R⁷ is independently halogen, —OR^{a}, —SR^{a}, C₁-C₆alkyl, cycloalkyl, or heterocycloalkyl.

In some embodiments of a compound of Formula (I) or (II),

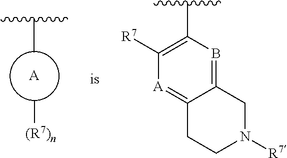

wherein:
A and B are independently selected from CH, N, and CF, with the proviso that at least one of A or B is N or CF.
R^{7′} is hydrogen or C₁-C₆alkyl; and
R⁷ is independently halogen, —OR^{a}, —SR^{a}, C₁-C₆alkyl, cycloalkyl, or heterocycloalkyl.

In some embodiments of a compound of Formula (I) or (II),

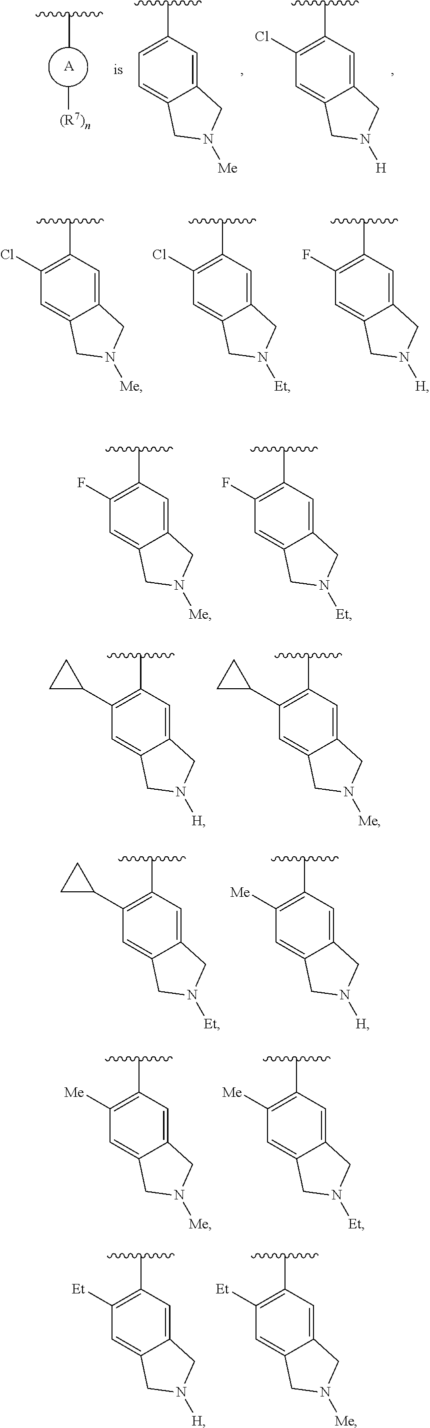

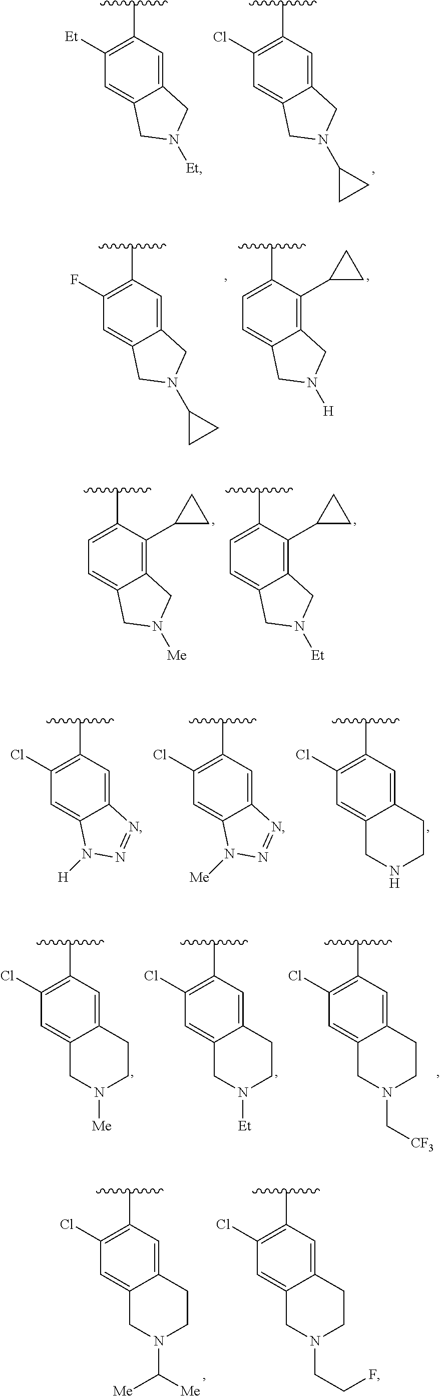

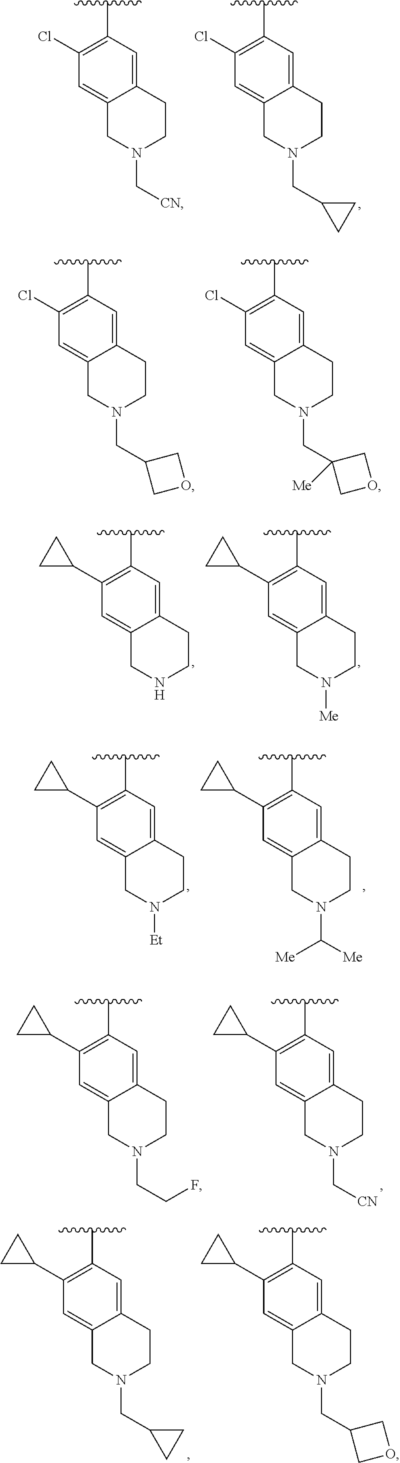

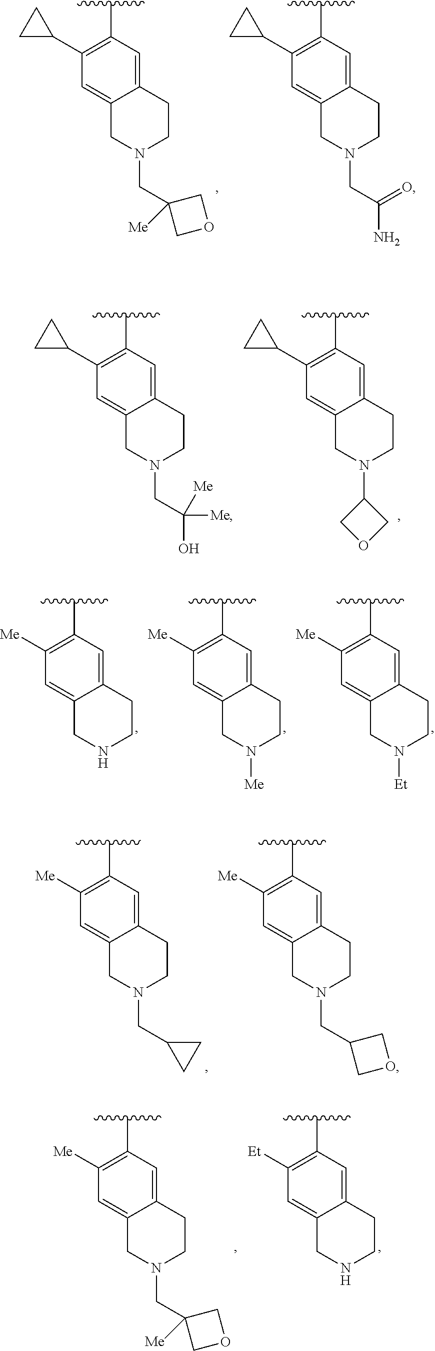

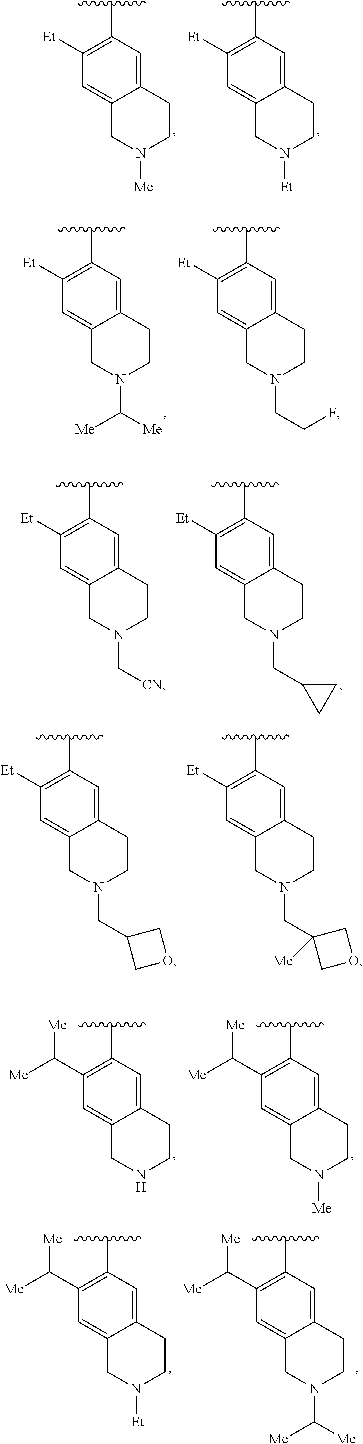

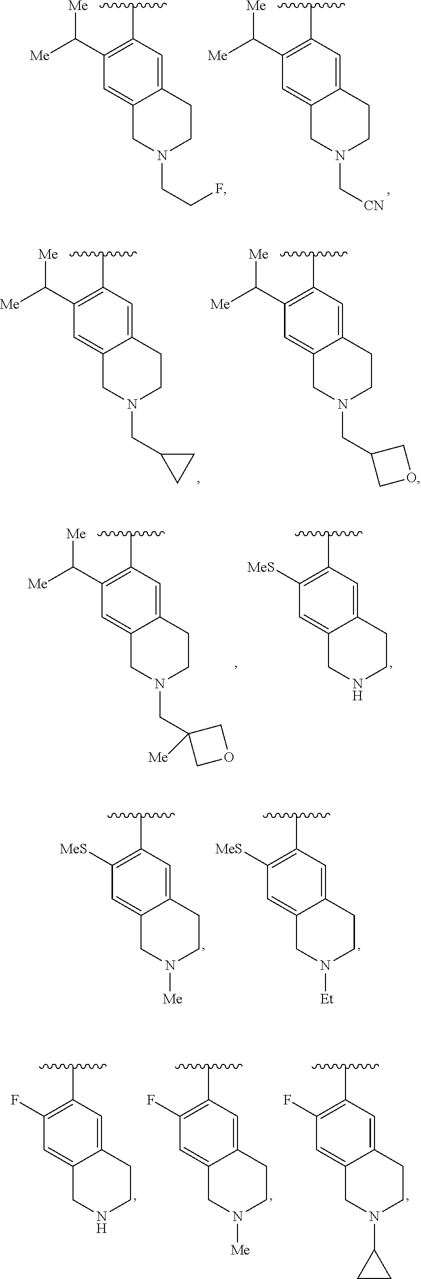

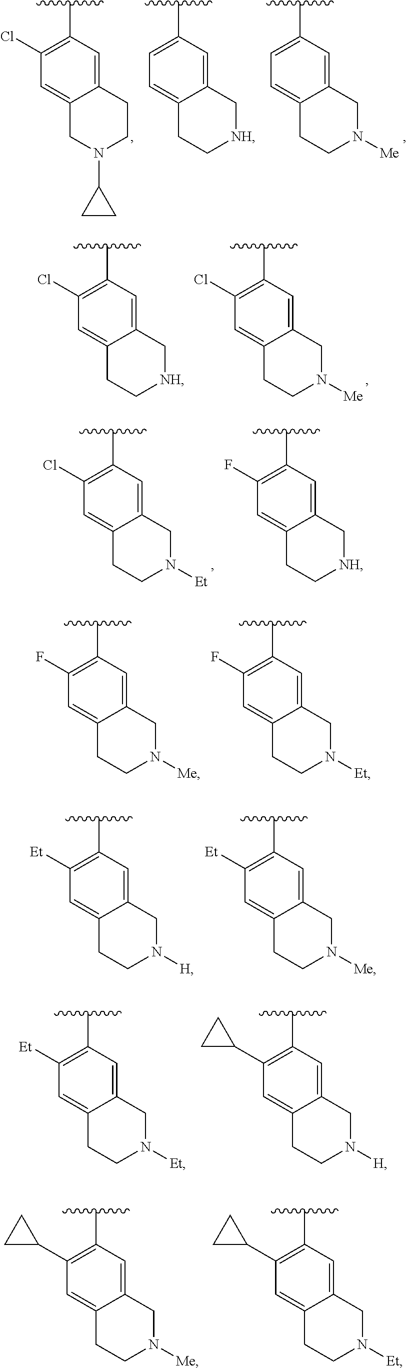

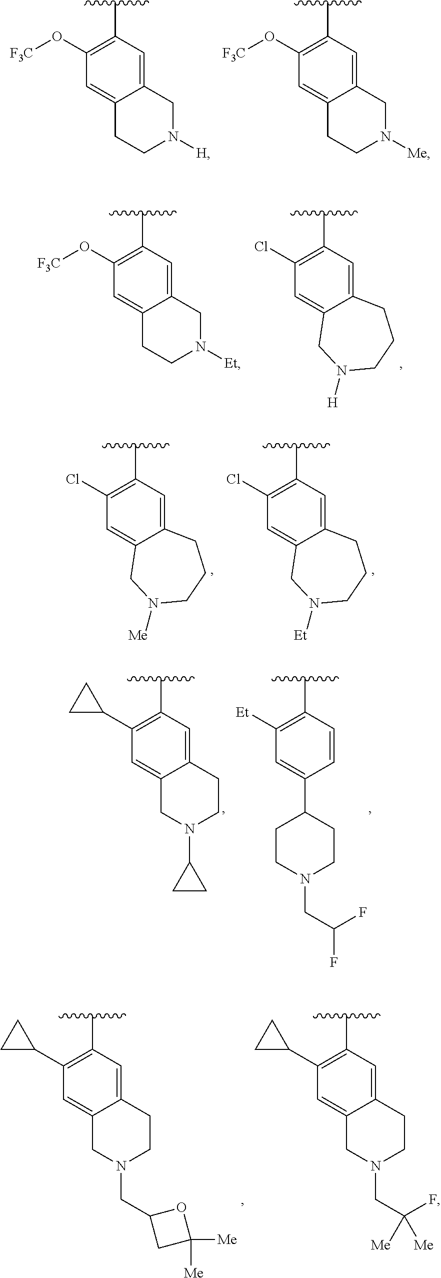

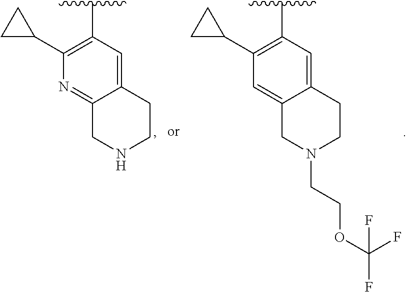 [SMILES: *c1cc2c(nc1C1CC1)CNCC2 ; *c1cc2c(cc1C1CC1)CN(CCOC(F)(F)F)CC2]

In some embodiments of a compound of Formula (I) or (II),

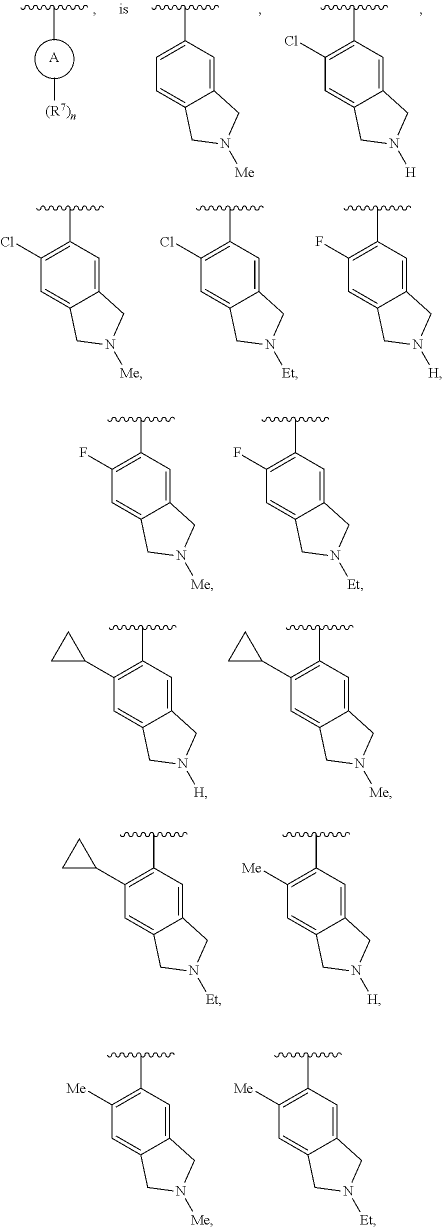

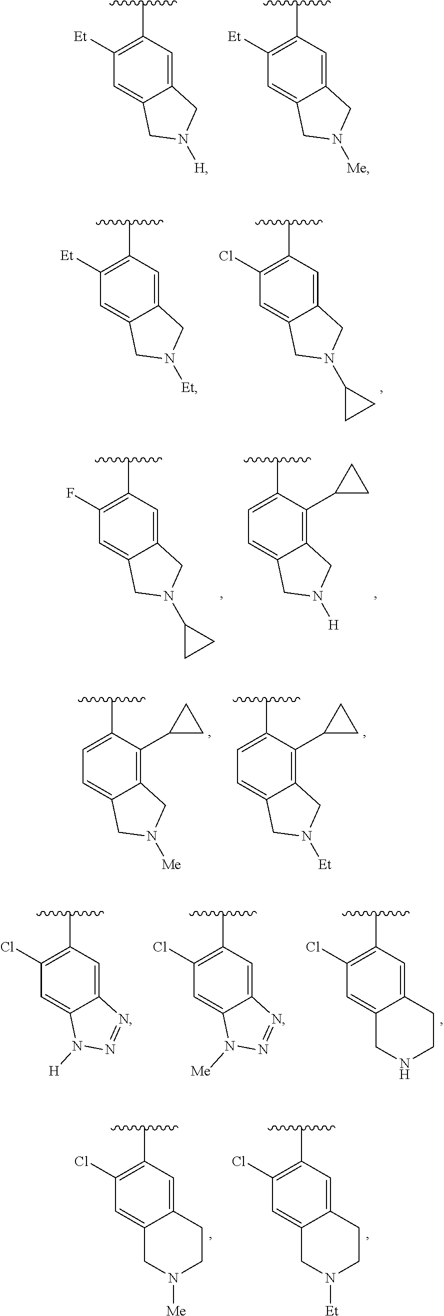

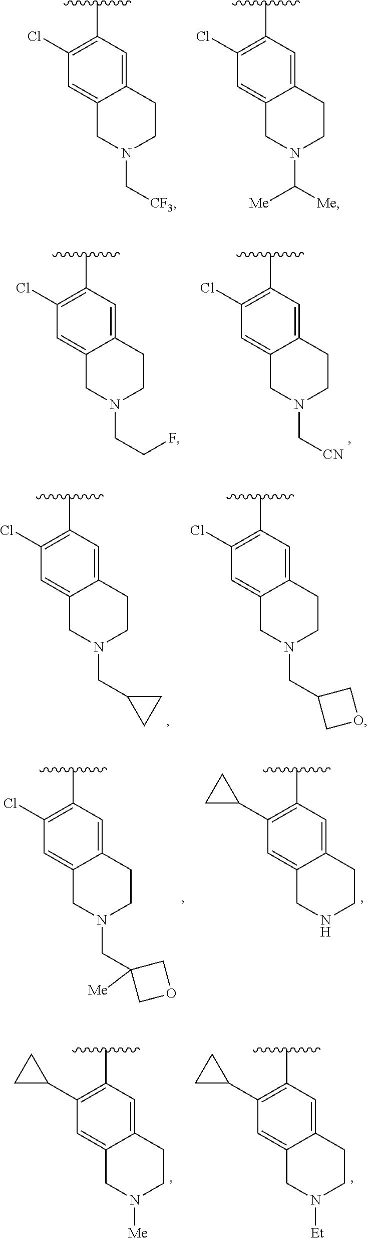

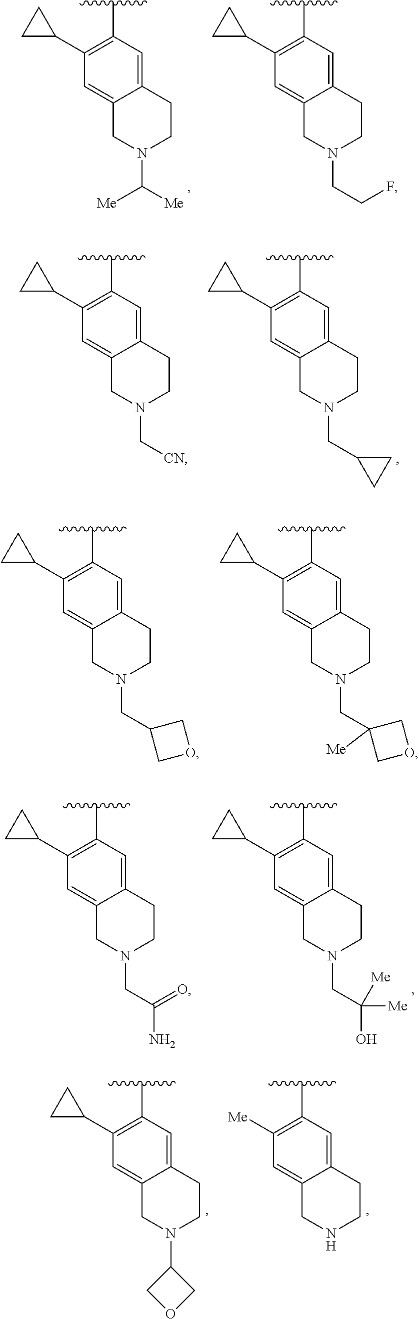

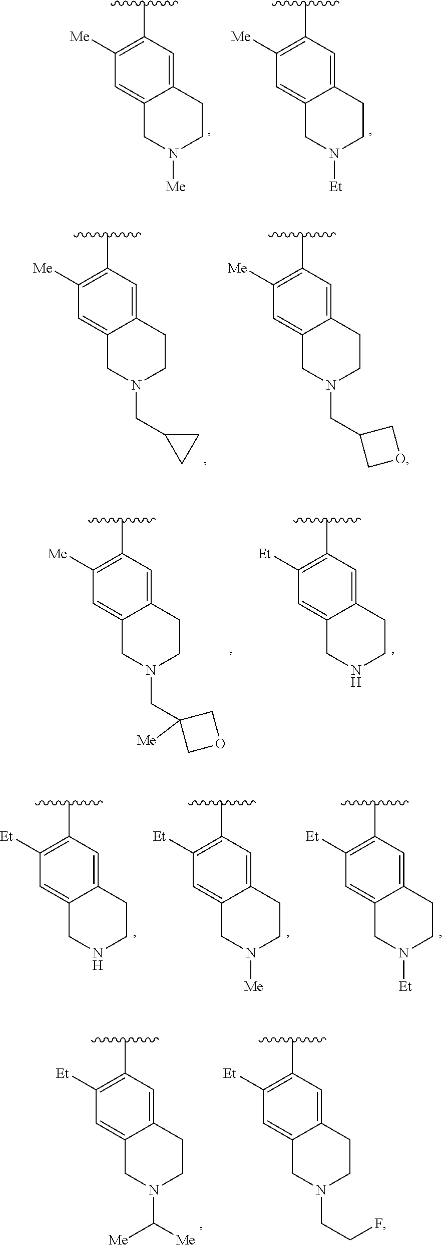

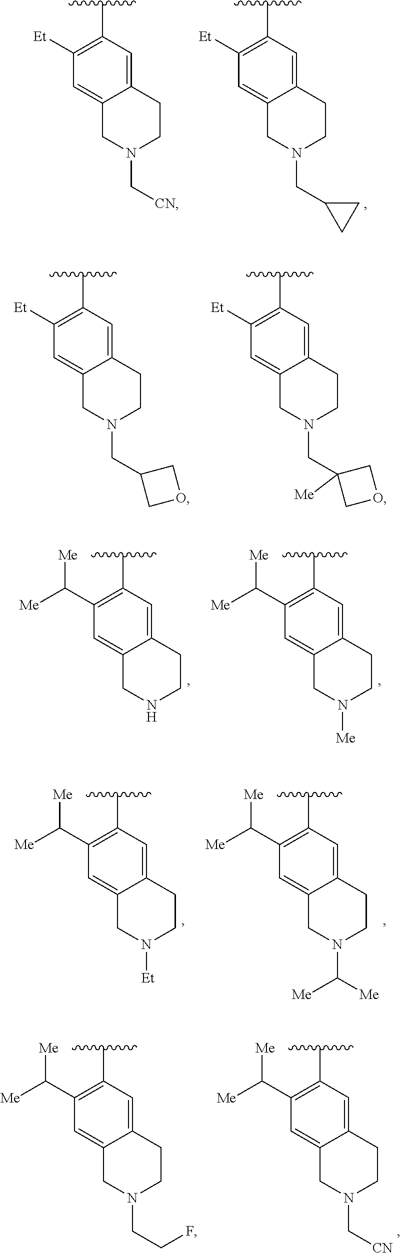

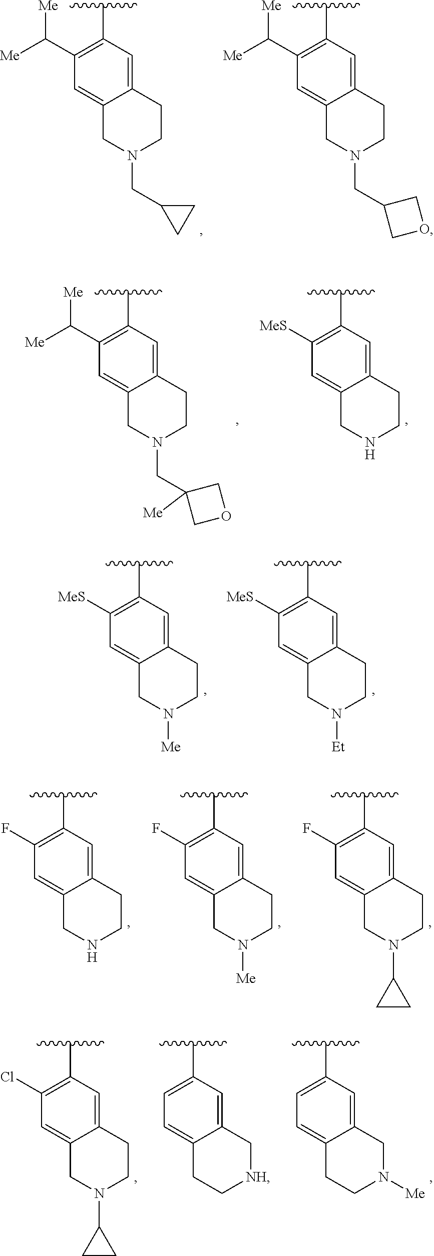

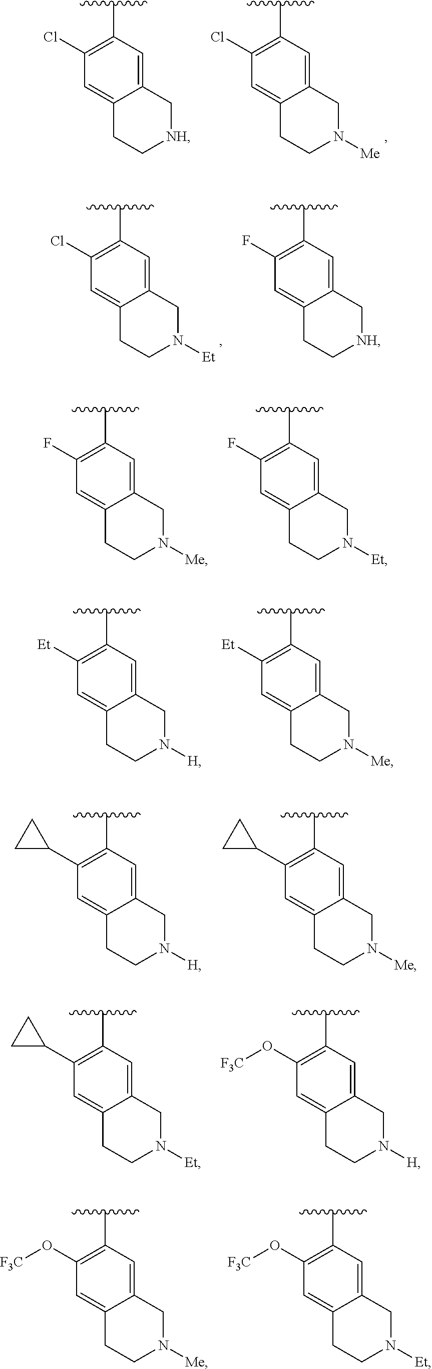

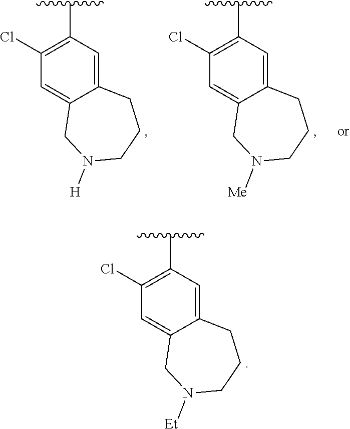 [SMILES: *c1cc2c(cc1Cl)CNCCC2 ; *c1cc2c(cc1Cl)CN(C)CCC2 ; *c1cc2c(cc1Cl)CN(CC)CCC2]

In some embodiments of a compound of Formula (I) or (II),

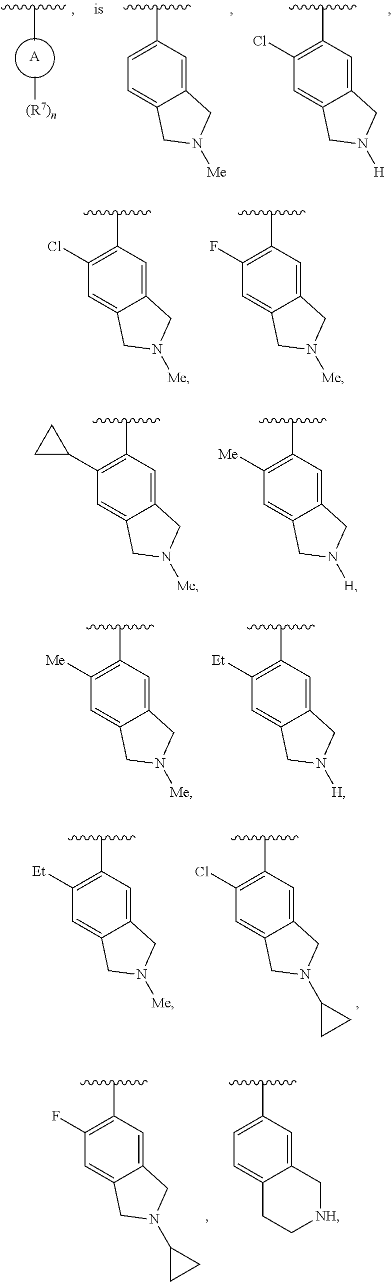

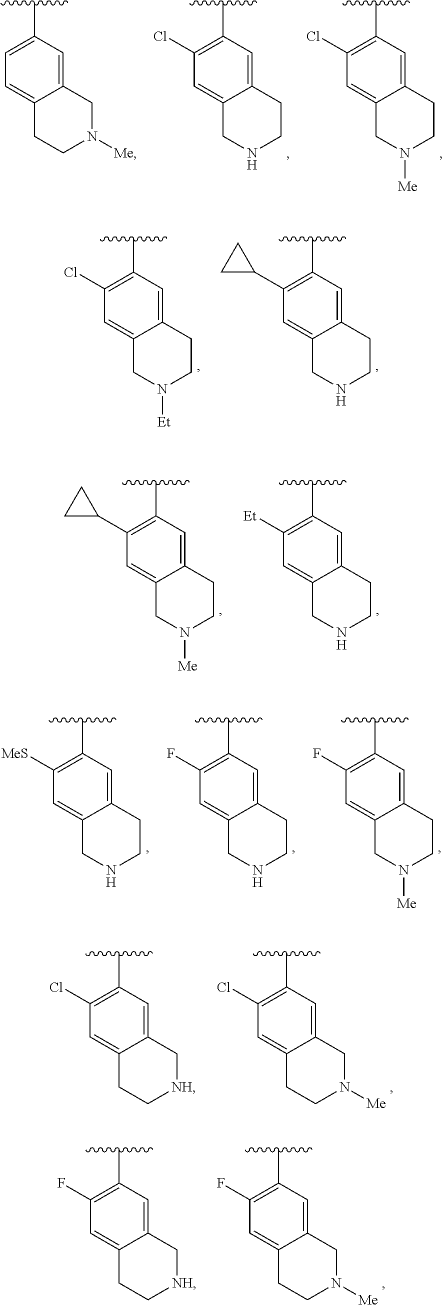

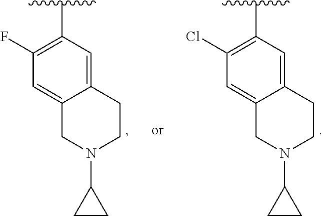 [SMILES: *c1cc2c(cc1F)CN(C1CC1)CC2 ; *c1cc2c(cc1Cl)CN(C1CC1)CC2]

In some embodiments of a compound of Formula (I) or (II),

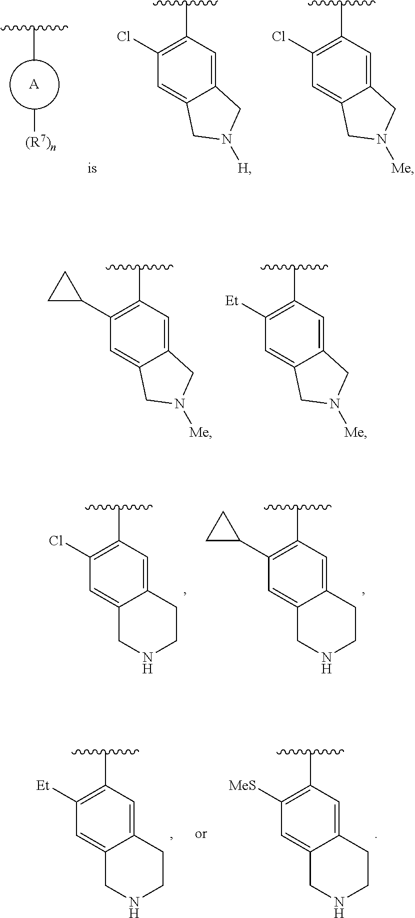

In some embodiments of a compound of Formula (I) or (II),

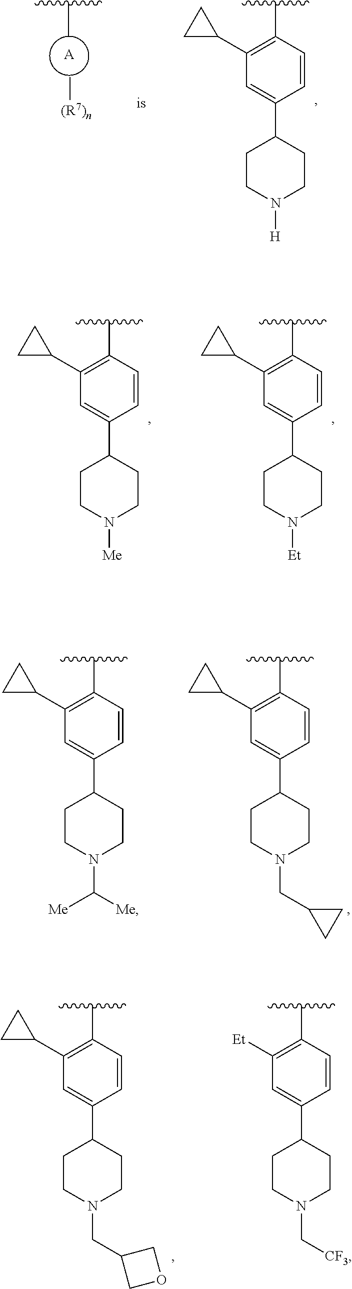

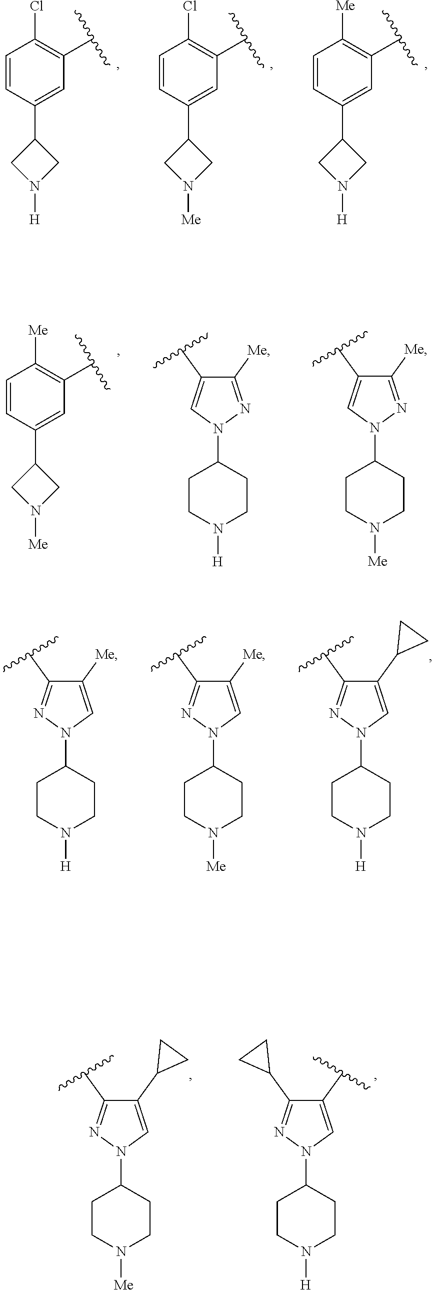

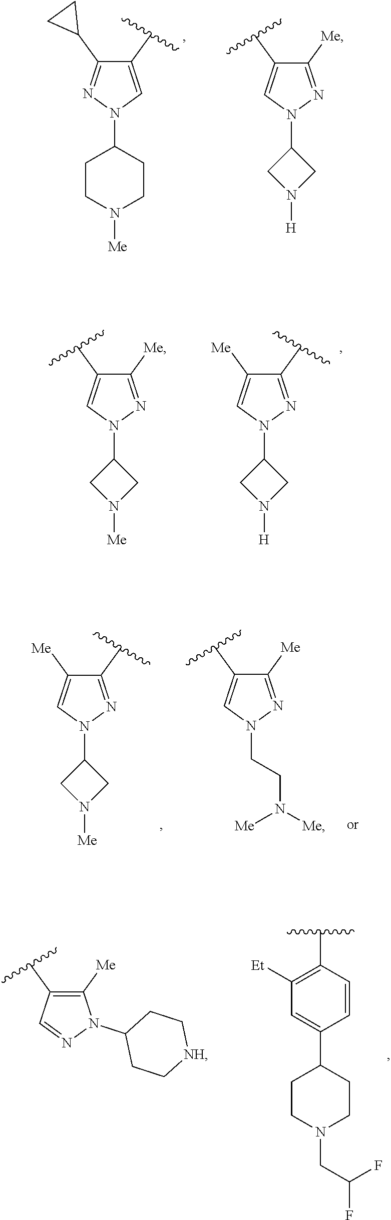

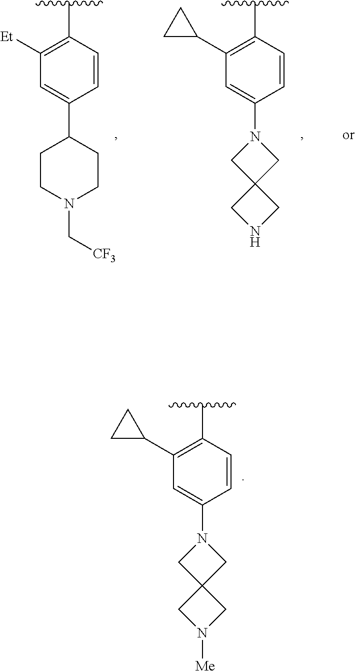 [SMILES: *c1ccc(C2CCN(CC(F)(F)F)CC2)cc1CC ; *c1ccc(N2CC3(CNC3)C2)cc1C1CC1 ; *c1ccc(N2CC3(CN(C)C3)C2)cc1C1CC1]

In some embodiments of a compound of Formula (I) or (II),

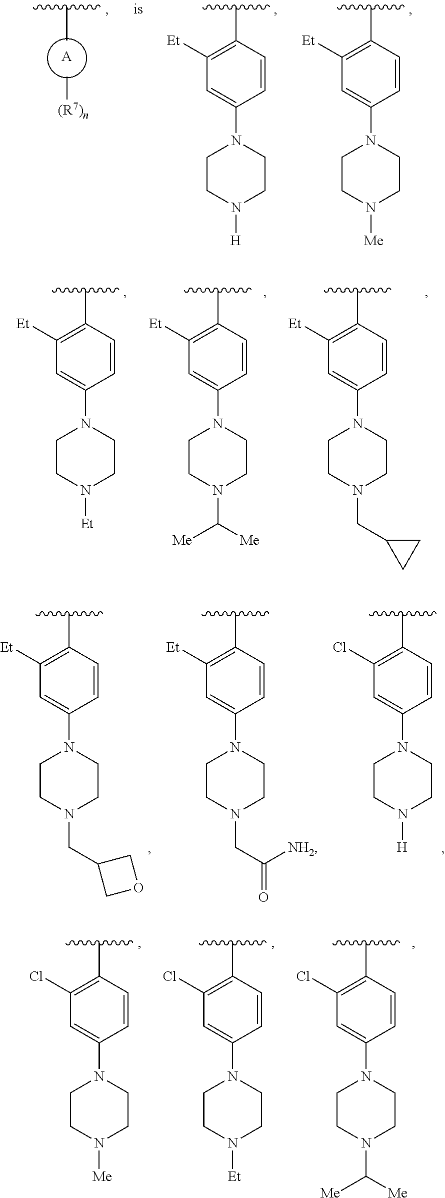

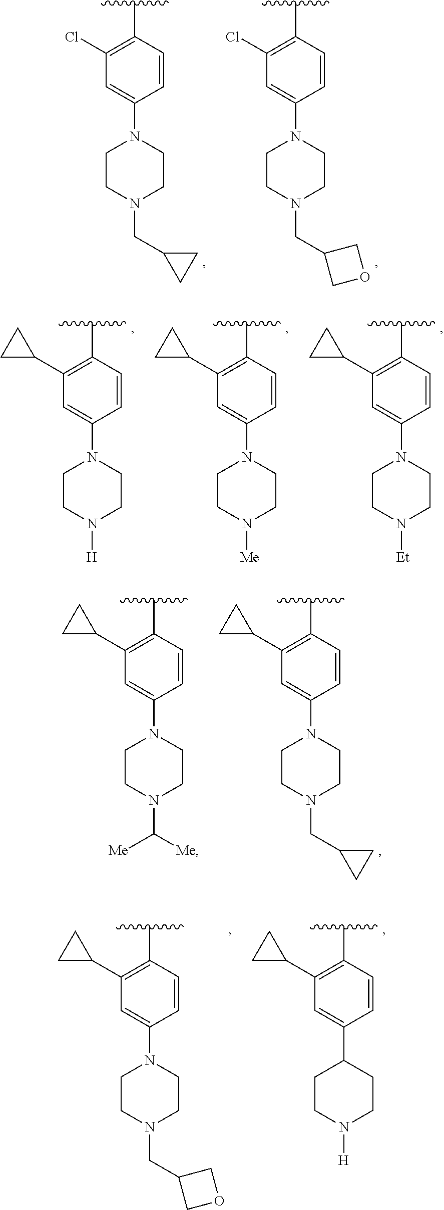

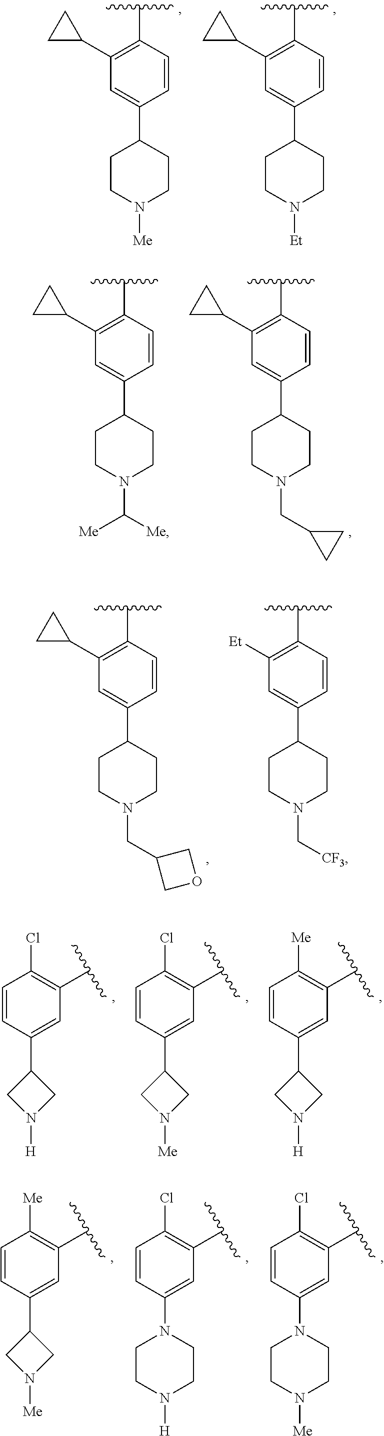

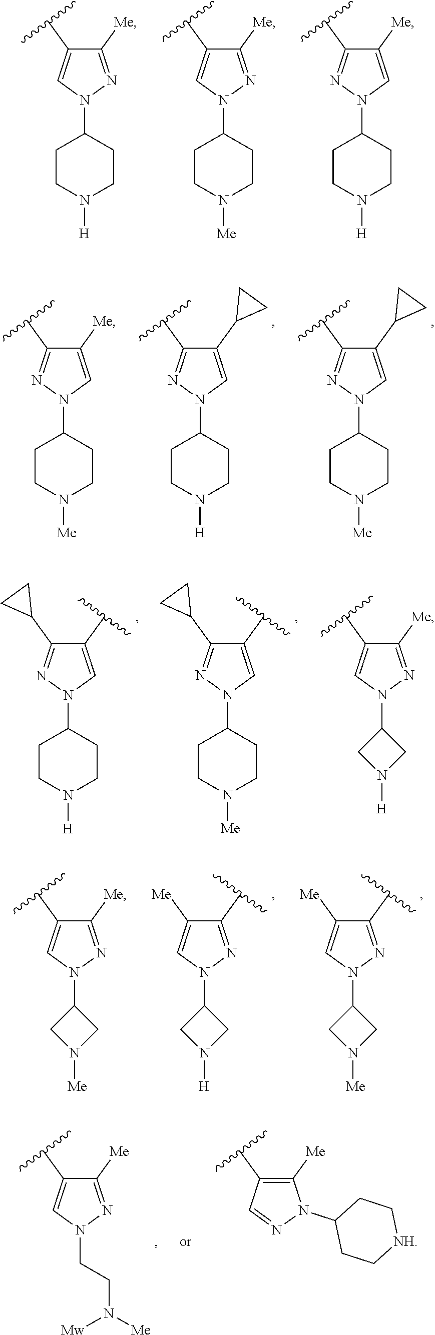

In some embodiments of a compound of Formula (I) or (II),

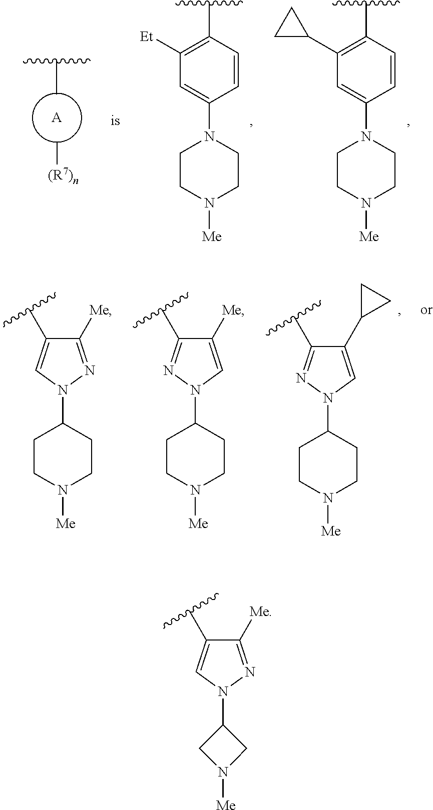

In some embodiments of a compound of Formula (I) or (II),

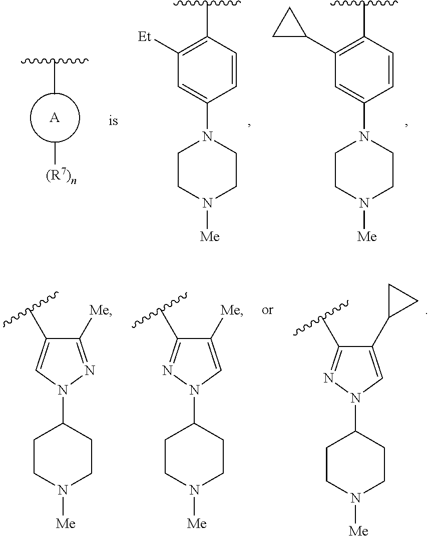

Disclosed herein is a compound of Formula (III), or a pharmaceutically acceptable salt thereof:

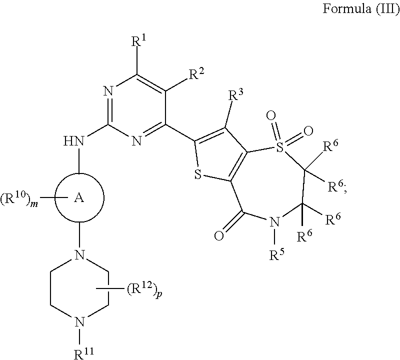

wherein
R¹ is hydrogen, halogen, C₁-C₆alkyl, C₁-C₆haloalkyl, C₁-C₆hydroxyalkyl, C₁-C₆aminoalkyl, or C₁-C₆heteroalkyl;
R² is hydrogen, halogen, C₁-C₆alkyl, C₁-C₆haloalkyl, C₁-C₆hydroxyalkyl, C₁-C₆aminoalkyl, C₁-C₆heteroalkyl, —C(═O)NR^{c}R^{d}, or cycloalkyl;
R³ is hydrogen, halogen, C₁-C₆alkyl, C₁-C₆haloalkyl, C₁-C₆hydroxyalkyl, C₁-C₆aminoalkyl, or C₁-C₆heteroalkyl;
R⁵ is hydrogen, —C(═O)R^{a}, —C(═O)OR^{b}, —C(═O)NR^{c}R^{d}, —S(═O)₂NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl is optionally and independently substituted with one or more R;
each R⁶ is independently hydrogen, halogen, —CN, —NO₂, —OH, —OR^{a}, —OC(═O)NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R⁶ on the same carbon are taken together to form an oxo;
or two R⁶ on the same carbon are taken together to form a cycloalkyl or a heterocycloalkyl; each optionally substituted with one or more R;
or two R⁶ on adjacent carbons are taken together to form a cycloalkyl, a heterocycloalkyl, an aryl, or a heteroaryl; each optionally substituted with one or more R;
Ring A is aryl or heteroaryl;
each R¹⁰ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆alkoxyC1-C₂alkylenyloxy, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkylenyl, alkenyl, alkynyl, alkoxy, cycloalkyl, heterocycloalkyl, aryl, and heteroaryl is optionally and independently substituted with one or more R;
m is 0-4;
R¹¹ is hydrogen, -L-S(═O)R^{a}, -L-S(═O)₂R^{a}, -L-S(═O)₂NR^{c}R^{d}, -L-C(═O)R^{a}, -L-C(═O)OR^{b}, -L-C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl; wherein the alkyl, alkenyl, alkynyl, cycloalkyl, heterocycloalkyl, aryl, and heteroaryl is optionally and independently substituted with one or more R;
L is absent or C₁-C₃ alkylene;
each R¹² is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R¹² on the same atom are taken together to form an oxo;
or two R¹² on the same carbon are taken together to form a cycloalkyl or a heterocycloalkyl; each optionally substituted with one or more R;
or two R¹² on different carbons are taken together to form a cycloalkyl, a heterocycloalkyl, an aryl, or a heteroaryl; each optionally substituted with one or more R;
p is 0-8;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
or two R on the same atom form an oxo.

In some embodiments of a compound of Formula (III), Ring A is heteroaryl. In some embodiments of a compound of Formula (III), Ring A is 5- or 6-membered heteroaryl. In some embodiments of a compound of Formula (III), Ring A is 5-membered heteroaryl. In some embodiments of a compound of Formula (III), Ring A is 6-membered heteroaryl. In some embodiments of a compound of Formula (III), Ring A is pyrazolyl. In some embodiments of a compound of Formula (III), Ring A is phenyl or pyridinyl. In some embodiments of a compound of Formula (III), Ring A is phenyl. In some embodiments of a compound of Formula (III), Ring A is phenyl.

In some embodiments of a compound of Formula (III), m is 1-4. In some embodiments of a compound of Formula (III), m is 1-3. In some embodiments of a compound of Formula (III), m is 1 or 2. In some embodiments of a compound of Formula (III), m is 0. In some embodiments of a compound of Formula (III), m is 1. In some embodiments of a compound of Formula (III), m is 2. In some embodiments of a compound of Formula (III), m is 3.

In some embodiments of a compound of Formula (III), the compound is of Formula (IIIa):

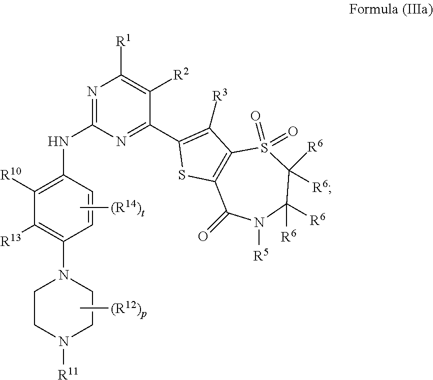

wherein:
R¹³ is hydrogen or R¹⁰;
each R¹⁴ is independently deuterium, halogen, —CN, C₁-C₆alkyl, or C₁-C₆haloalkyl; and
t is 0-2.

In some embodiments of a compound of Formula (IIIa), R¹³ is R¹⁰.

In some embodiments of a compound of Formula (III), the compound is of Formula (IIIb):

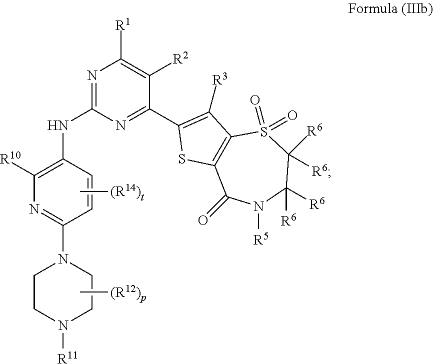

wherein:
each R¹⁴ is independently deuterium, halogen, —CN, C₁-C₆alkyl, or C₁-C₆haloalkyl; and
t is 0-2.

In some embodiments of a compound of Formula (IIIa) or (IIIb), each R¹⁴ is independently deuterium, halogen, —CN, CH₃, or CF₃. In some embodiments of a compound of Formula (IIIa) or (IIIb), each R¹⁴ is independently fluorine.

In some embodiments of a compound of Formula (IIIa) or (IIIb), t is 0. In some embodiments of a compound of Formula (IIIa) or (IIIb), t is 1. In some embodiments of a compound of Formula (IIIa) or (IIIb), t is 2.

In some embodiments of a compound of Formula (III), the compound is of Formula (IIIc):

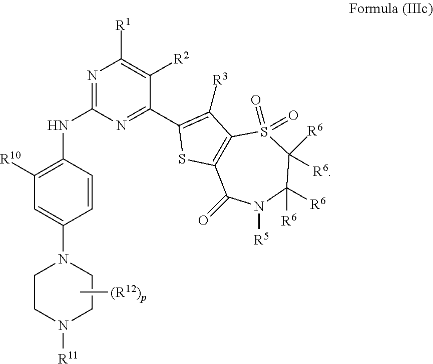

In some embodiments of a compound of Formula (III), the compound is of Formula (IIId):

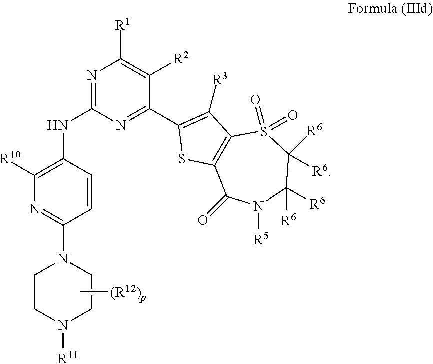

In some embodiments of a compound of Formula (III) or (IIIa)-(IIId), each R¹⁰ is independently halogen, —CN, —OH, —OR^{a}, —SH, —SR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally and independently substituted with one or more R.

In some embodiments of a compound of Formula (III) or (IIIa)-(IIId), each R¹⁰ is independently halogen, —CN, C₁-C₆alkyl, C₁-C₆haloalkyl, cycloalkyl, or heterocycloalkyl; wherein the alkyl, cycloalkyl, and heterocycloalkyl is optionally and independently substituted with one or more R. In some embodiments of a compound of Formula (III) or (IIIa)-(IIId), each R¹⁰ is independently halogen, C₁-C₆alkyl, or cycloalkyl; wherein the alkyl and cycloalkyl is optionally and independently substituted with one or more R. In some embodiments of a compound of Formula (III) or (IIIa)-(IIId), each R¹⁰ is independently halogen, C₁-C₆alkyl, or cycloalkyl. In some embodiments of a compound of Formula (III) or (IIIa)-(IIId), each R¹⁰ is independently halogen. In some embodiments of a compound of Formula (III) or (IIIa)-(IIId), each R¹⁰ is independently C₁-C₆alkyl. In some embodiments of a compound of Formula (III) or (IIIa)-(IIId), each R¹⁰ is independently cycloalkyl.

In some embodiments of a compound of Formula (III) or (IIIa)-(IIId), R¹ is hydrogen, halogen, C₁-C₆alkyl, or C₁-C₆haloalkyl. In some embodiments of a compound of Formula (III) or (IIIa)-(IIId), R¹ is hydrogen or C₁-C₂alkyl. In some embodiments of a compound of Formula (III) or (IIIa)-(IIId), R¹ is hydrogen. In some embodiments of a compound of Formula (III) or (IIIa)-(IIId), R¹ is C₁-C₄alkyl. In some embodiments of a compound of Formula (III) or (IIIa)-(IIId), R¹ is C₁-C₃alkyl. In some embodiments of a compound of Formula (III) or (IIIa)-(IIId), R¹ is C₁-C₂alkyl. In some embodiments of a compound of Formula (III) or (IIIa)-(IIId), R¹ is CH₃.

In some embodiments of a compound of Formula (III) or (IIIa)-(IIId), R² is hydrogen, halogen, C₁-C₆alkyl, C₁-C₆haloalkyl, —C(═O)NR^{c}R^{d}, or cycloalkyl. In some embodiments of a compound of Formula (III) or (IIIa)-(IIId), R² is C₁-C₆alkyl, C₁-C₆haloalkyl, —C(═O)NR^{c}R^{d}, or cycloalkyl. In some embodiments of a compound of Formula (III) or (IIIa)-(IIId), R² is C₁-C₆alkyl or C₁-C₆haloalkyl. In some embodiments of a compound of Formula (III) or (IIIa)-(IIId), R² is C₁-C₆haloalkyl. In some embodiments of a compound of Formula (III) or (IIIa)-(IIId), R² is C₁-C₂alkyl, C₁-C₂haloalkyl, —C(═O)NR^{c}R^{d}, or cycloalkyl. In some embodiments of a compound of Formula (III) or (IIIa)-(IIId), R² is C₁-C₂alkyl or C₁-C₂haloalkyl. In some embodiments of a compound of Formula (III) or (IIIa)-(IIId), R² is CF₃. In some embodiments of a compound of Formula (III) or (IIIa)-(IIId), R² is C₁-C₄alkyl. In some embodiments of a compound of Formula (III) or (IIIa)-(IIId), R² is C₁-C₃alkyl. In some embodiments of a compound of Formula (III) or (IIIa)-(IIId), R² is C₁-C₂alkyl. In some embodiments of a compound of Formula (III) or (IIIa)-(IIId), R² is CH₃. In some embodiments of a compound of Formula (III) or (IIIa)-(IIId), R² is cyclopropyl. In some embodiments of a compound of Formula (III) or (IIIa)-(IIId), R² is —C(═O)NH₂.

In some embodiments of a compound of Formula (III) or (IIIa)-(IIId), R³ is hydrogen, halogen, C₁-C₆alkyl, or C₁-C₆haloalkyl. In some embodiments of a compound of Formula (III) or (IIIa)-(IIId), R³ is hydrogen.

In some embodiments of a compound of Formula (III) or (IIIa)-(IIId), R⁵ is hydrogen, —C(═O)R^{a}, —C(═O)OR^{b}, —C(═O)NR^{c}R^{d}, —S(═O)₂NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl is optionally and independently substituted with one or more R. In some embodiments of a compound of Formula (III) or (IIIa)-(IIId), R⁵ is hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkyl(cycloalkyl), or C₁-C₆alkyl(heterocycloalkyl); wherein the alkyl, cycloalkyl, and heterocycloalkyl is optionally and independently substituted with one or more R. In some embodiments of a compound of Formula (III) or (IIIa)-(IIId), R is hydrogen, C₁-C₆alkyl, C₁-C₆haloalkyl, cycloalkyl, or heterocycloalkyl; wherein the alkyl, cycloalkyl, and heterocycloalkyl is optionally and independently substituted with one or more R. In some embodiments of a compound of Formula (III) or (IIIa)-(IIId), R⁵ is hydrogen, C₁-C₆alkyl, C₁-C₆haloalkyl, cycloalkyl, or heterocycloalkyl. In some embodiments of a compound of Formula (III) or (IIIa)-(IIId), R is hydrogen, C₁-C₆alkyl, cycloalkyl, or heterocycloalkyl. In some embodiments of a compound of Formula (III) or (IIIa)-(IIId), R⁵ is hydrogen, methyl, cyclopropyl, or oxetanyl.

In some embodiments of a compound of Formula (III) or (IIIa)-(IIId), each R⁶ is independently hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally and independently substituted with one or more R. In some embodiments of a compound of Formula (III) or (IIIa)-(IIId), each R⁶ is independently hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (III) or (IIIa)-(IIId), each R⁶ is independently hydrogen, halogen, C₁-C₆alkyl, or C₁-C₆haloalkyl. In some embodiments of a compound of Formula (III) or (IIIa)-(IIId), each R⁶ is independently hydrogen or C₁-C₆alkyl. In some embodiments of a compound of Formula (III) or (IIIa)-(IIId), each R⁶ is hydrogen. In some embodiments of a compound of Formula (III) or (IIIa)-(IIId), two R⁶ on the same carbon are taken together to form a cycloalkyl or a heterocycloalkyl; each optionally substituted with one or more R. In some embodiments of a compound of Formula (III) or (IIIa)-(IIId), two R⁶ on adjacent carbons are taken together to form a cycloalkyl, a heterocycloalkyl, an aryl, or a heteroaryl; each optionally substituted with one or more R.

In some embodiments of a compound of Formula (III) or (IIIa)-(IIId), R¹¹ is hydrogen, -L-S(═O)R^{a}, -L-S(═O)₂R^{a}, -L-S(═O)₂NR^{c}R^{d}, -L-C(═O)R^{a}, -L-C(═O)OR^{b}, -L-C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl; wherein the alkyl, alkenyl, alkynyl, cycloalkyl, heterocycloalkyl, aryl, and heteroaryl is optionally and independently substituted with one or more R. In some embodiments of a compound of Formula (III) or (IIIa)-(IIId), R¹¹ is hydrogen, -L-S(═O)R^{a}, -L-S(═O)₂R^{a}, -L-S(═O)₂NR^{c}R^{d}, -L-C(═O)R^{a}, -L-C(═O)OR^{b}, -L-C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, -L-cycloalkyl, or -L-heterocycloalkyl; wherein the alkyl, cycloalkyl, and heterocycloalkyl is optionally and independently substituted with one or more R. In some embodiments of a compound of Formula (III) or (IIIa)-(IIId), R¹¹ is hydrogen, -L-C(═O)OR^{b}, C₁-C₆alkyl, C₁-C₆haloalkyl, C₁-C₆hydroxyalkyl, C₁-C₆aminoalkyl, C₁-C₆heteroalkyl, -L-cycloalkyl, or -L-heterocycloalkyl; wherein the alkyl, cycloalkyl, and heterocycloalkyl is optionally and independently substituted with one or more R. In some embodiments of a compound of Formula (III) or (IIIa)-(IIId), R¹¹ is hydrogen, C₁-C₆alkyl, C₁-C₆haloalkyl, -L-cycloalkyl, or -L-heterocycloalkyl; wherein the alkyl, cycloalkyl, and heterocycloalkyl is optionally and independently substituted with one or more R. In some embodiments of a compound of Formula (III) or (IIIa)-(IIId), R¹¹ is hydrogen, C₁-C₆alkyl, -L-cycloalkyl, or -L-heterocycloalkyl; wherein the alkyl, cycloalkyl, and heterocycloalkyl is optionally and independently substituted with one or more R. In some embodiments of a compound of Formula (III) or (IIIa)-(IIId), R¹¹ is hydrogen, C₁-C₆alkyl, -L-cycloalkyl, or -L-heterocycloalkyl. In some embodiments of a compound of Formula (III) or (IIIa)-(IIId), R¹¹ is C₁-C₆alkyl optionally substituted with one or more R. In some embodiments of a compound of Formula (III) or (IIIa)-(IIId), R¹¹ is -L-cycloalkyl optionally substituted with one or more R. In some embodiments of a compound of Formula (III) or (IIIa)-(IIId), R¹¹ is -L-heterocycloalkyl optionally substituted with one or more R.

In some embodiments of a compound of Formula (III) or (IIIa)-(IIId), L is absent. In some embodiments of a compound of Formula (III) or (IIIa)-(IIId), L is C₁-C₃ alkylene. In some embodiments of a compound of Formula (III) or (IIIa)-(IIId), L is CH₂.

In some embodiments of a compound of Formula (III) or (IIIa)-(IIId), each R¹² is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally and independently substituted with one or more R. In some embodiments of a compound of Formula (III) or (IIIa)-(IIId), each R¹² is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (III) or (IIIa)-(IIId), each R¹² is independently halogen, C₁-C₆alkyl, C₁-C₆haloalkyl, C₁-C₆hydroxyalkyl, C₁-C₆aminoalkyl, or C₁-C₆heteroalkyl. In some embodiments of a compound of Formula (III) or (IIIa)-(IIId), each R¹² is independently C₁-C₆alkyl, C₁-C₆haloalkyl, or C₁-C₆hydroxyalkyl. In some embodiments of a compound of Formula (III) or (IIIa)-(IIId), each R¹² is independently C₁-C₆alkyl, or C₁-C₆hydroxyalkyl. In some embodiments of a compound of Formula (III) or (IIIa)-(IIId), each R¹² is independently C₁-C₆alkyl. In some embodiments of a compound of Formula (III) or (IIIa)-(IIId), two R¹² on the same carbon are taken together to form a cycloalkyl or a heterocycloalkyl; each optionally substituted with one or more R. In some embodiments of a compound of Formula (III) or (IIIa)-(IIId), two R¹² on different carbons are taken together to form a cycloalkyl, a heterocycloalkyl, an aryl, or a heteroaryl; each optionally substituted with one or more R. In some embodiments of a compound of Formula (III) or (IIIa)-(IIId), two R¹² on different carbons are taken together to form a cycloalkyl or a heterocycloalkyl; each optionally substituted with one or more R.

In some embodiments of a compound of Formula (III) or (IIIa)-(IIId), two R¹² on different carbons are taken together to form a cycloalkyl or a heterocycloalkyl. In some embodiments of a compound of Formula (III) or (IIIa)-(IIId), two R¹² on different carbons are taken together to form a cycloalkyl. In some embodiments of a compound of Formula (III) or (IIIa)-(IIId), two R¹² on different carbons are taken together to form a heterocycloalkyl.

In some embodiments of a compound of Formula (III) or (IIIa)-(IIId), p is 0-4. In some embodiments of a compound of Formula (III) or (IIIa)-(IIId), p is 0-3. In some embodiments of a compound of Formula (III) or (IIIa)-(IIId), p is 0-2. In some embodiments of a compound of Formula (III) or (IIIa)-(IIId), p is 0 or 1. In some embodiments of a compound of Formula (III) or (IIIa)-(IIId), p is 1-4. In some embodiments of a compound of Formula (III) or (IIIa)-(IIId), p is 1-3. In some embodiments of a compound of Formula (III) or (IIIa)-(IIId), p is 1 or 2. In some embodiments of a compound of Formula (III) or (IIIa)-(IIId), p is 0. In some embodiments of a compound of Formula (III) or (IIIa)-(IIId), p is 1. In some embodiments of a compound of Formula (III) or (IIIa)-(IIId), p is 2. In some embodiments of a compound of Formula (III) or (IIIa)-(IIId), p is 3. In some embodiments of a compound of Formula (III) or (IIIa)-(IIId), p is 4. In some embodiments of a compound of Formula (III) or (IIIa)-(IIId), p is 5. In some embodiments of a compound of Formula (III) or (IIIa)-(IIId), p is 6.

In some embodiments of a compound of Formula (III) or (IIIa)-(IIId),

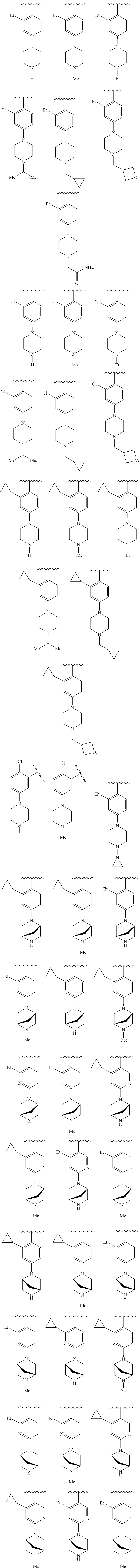

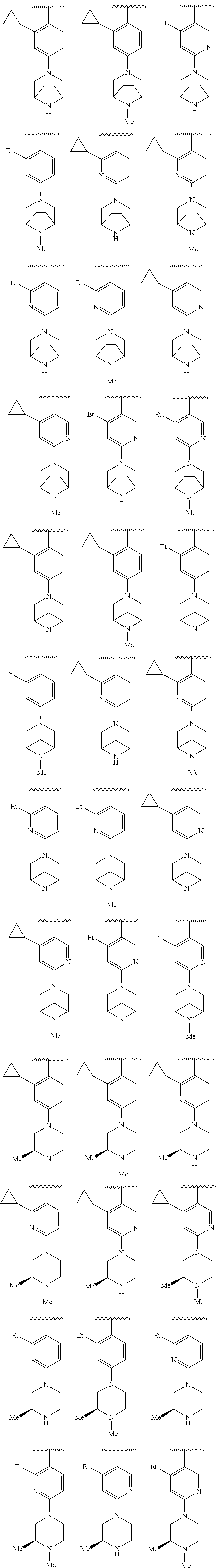

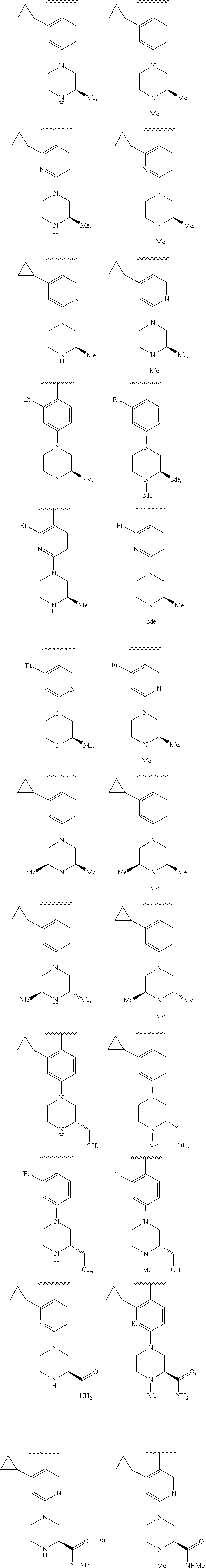

In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl, wherein the alkyl, cycloalkyl, and heterocycloalkyl is independently optionally substituted with one or more R. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl.

In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl, wherein the alkyl, cycloalkyl, and heterocycloalkyl is independently optionally substituted with one or more R. In some embodiments of a compound disclosed herein, each R^{b} is independently hydrogen, C₁-C₆alkyl or C₁-C₆haloalkyl. In some embodiments of a compound disclosed herein, each R^{b} is independently hydrogen or C₁-C₆alkyl. In some embodiments of a compound disclosed herein, each R^{b} is hydrogen. In some embodiments of a compound disclosed herein, each R^{b} is independently C₁-C₆alkyl.

In some embodiments of a compound disclosed herein, R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl, wherein the alkyl, cycloalkyl, and heterocycloalkyl is independently optionally substituted with one or more R. In some embodiments of a compound disclosed herein, R^{c} and R^{d} are each independently hydrogen, C₁-C₆alkyl or C₁-C₆haloalkyl. In some embodiments of a compound disclosed herein, R^{c} and R^{d} are each independently hydrogen or C₁-C₆alkyl. In some embodiments of a compound disclosed herein, R^{c} and R^{d} are each hydrogen. In some embodiments of a compound disclosed herein, R^{c} and R^{d} are each independently C₁-C₆alkyl.

In some embodiments of a compound disclosed herein, R^{c} and R^{d} are taken together with the atom to which they are attached to form a heterocycloalkyl optionally substituted with one or more R.

In some embodiments of a compound disclosed herein, each R is independently halogen, —CN, —OH, —OCH₃, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, and C₁-C₆heteroalkyl; or two R on the same atom form an oxo. In some embodiments of a compound disclosed herein, each R is independently halogen, —CN, —OH, —OCH₃, —NH₂, —N(CH₃)₂, —C(═O)CH₃, —C(═O)OH, —C(═O)OCH₃, C₁-C₆alkyl, or C₁-C₆haloalkyl; or two R on the same atom form an oxo. In some embodiments of a compound disclosed herein, each R is independently halogen, —CN, —OH, —OCH₃, —NH₂, —N(CH₃)₂, C₁-C₆alkyl, or C₁-C₆haloalkyl; or two R on the same atom form an oxo.

In some embodiments of a compound disclosed herein, each R⁶, R⁷, R^{7a}, R^{7b}, R⁸, R⁹, R¹⁰, R¹¹, R¹², R^{a}, R^{b}, R^{c}, R^{d}, the ring formed when: two R⁷ are taken together, two R⁶ are taken together, two R¹² are taken together, and R^{c} and R^{d} are taken together, is optionally and independently substituted with one, two, three, or four substituents as defined herein. In some embodiments of a compound disclosed herein, each R⁶, R⁷, R^{7a}, R^{7b}, R⁸, R⁹, R¹⁰, R¹¹, R¹², R^{a}, R^{b}, R^{c}, R^{d}, the ring formed when: two R⁷ are taken together, two R⁶ are taken together, two R¹² are taken together, and R^{c} and R^{d} are taken together, is optionally and independently substituted with one, two, or three substituents as defined herein. In some embodiments of a compound disclosed herein, each R⁶, R⁷, R^{7a}, R^{7b}, R⁸, R⁹, R¹⁰, R¹¹, R¹², R^{a}, R^{b}, R^{c}, R^{d}, the ring formed when: two R⁷ are taken together, two R⁶ are taken together, two R¹² are taken together, and R^{c} and R^{d} are taken together, is optionally and independently substituted with one, or two substituents as defined herein.

Any combination of the groups described above for the various variables is contemplated herein. Throughout the specification, groups and substituents thereof are chosen by one skilled in the field to provide stable moieties and compounds.

In some embodiments the compound disclosed herein, or a pharmaceutically acceptable salt, solvate, or stereoisomer thereof, is one of the compounds in Table 1.

TABLE 1

| Ex. | Structure | Name |
|---|---|---|
| 1 | 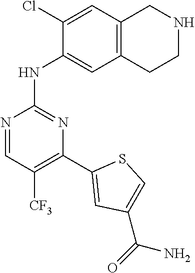 [SMILES: NC(=O)c1csc(-c2nc(Nc3cc4c(cc3Cl)CNCC4)ncc2C(F)(F)F)c1] | 5-(2-((7-chloro-1,2,3,4- tetrahydroisoquinolin-6-yl)amino)- 5-(trifluoromethyl)pyrimidin-4- yl)thiophene-3-carboxamide |
| 2 | 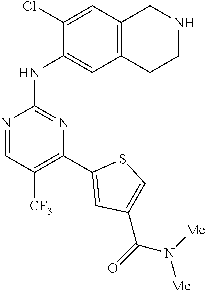 [SMILES: CN(C)C(=O)c1csc(-c2nc(Nc3cc4c(cc3Cl)CNCC4)ncc2C(F)(F)F)c1] | 5-(2-((7-chloro-1,2,3,4- tetrahydroisoquinolin-6-yl)amino)- 5-(trifluoromethyl)pyrimidin-4-yl)- N,N-dimethylthiophene-3- carboxamide |
| 3 | 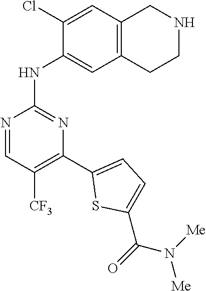 [SMILES: CN(C)C(=O)c1ccc(-c2nc(Nc3cc4c(cc3Cl)CNCC4)ncc2C(F)(F)F)s1] | 5-(2-((7-chloro-1,2,3,4- tetrahydroisoquinolin-6-yl)amino)- 5-(trifluoromethyl)pyrimidin-4-yl)- N,N-dimethylthiophene-2- carboxamide |
| 4 | 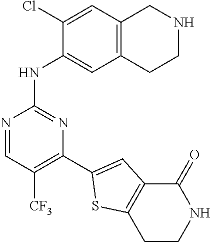 [SMILES: O=C1NCCc2sc(-c3nc(Nc4cc5c(cc4Cl)CNCC5)ncc3C(F)(F)F)cc21] | 2-(2-((7-chloro-1,2,3,4- tetrahydroisoquinolin-6-yl)amino)- 5-(trifluoromethyl)pyrimidin-4-yl)- 6,7-dihydrothieno[3,2-c]pyridin- 4(5H)-one |
| 5 | 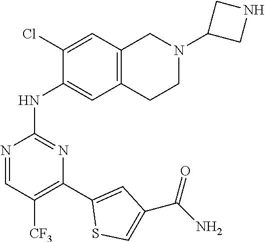 [SMILES: NC(=O)c1csc(-c2nc(Nc3cc4c(cc3Cl)CN(C3CNC3)CC4)ncc2C(F)(F)F)c1] | 5-(2-((2-(azetidin-3-yl)-7-chloro- 1,2,3,4-tetrahydroisoquinolin-6- yl)amino)-5- (trifluoromethyl)pyrimidin-4- yl)thiophene-3-carboxamide |
| 6 | 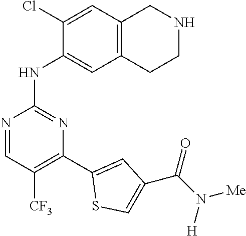 [SMILES: CNC(=O)c1csc(-c2nc(Nc3cc4c(cc3Cl)CNCC4)ncc2C(F)(F)F)c1] | 5-(2-((7-chloro-1,2,3,4- tetrahydroisoquinolin-6-yl)amino)- 5-(trifluoromethyl)pyrimidin-4-yl)- N-methylthiophene-3-carboxamide |
| 7 | 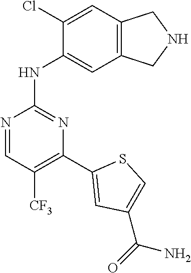 [SMILES: NC(=O)c1csc(-c2nc(Nc3cc4c(cc3Cl)CNC4)ncc2C(F)(F)F)c1] | 5-(2-((6-chloroisoindolin-5- yl)amino)-5- (trifluoromethyl)pyrimidin-4- yl)thiophene-3-carboxamide |
| 8 | 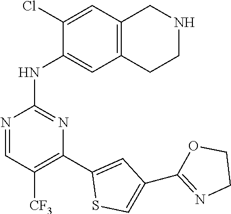 [SMILES: FC(F)(F)c1cnc(Nc2cc3c(cc2Cl)CNCC3)nc1-c1cc(C2=NCCO2)cs1] | 7-chloro-N-(4-(4-(4,5- dihydrooxazol-2-yl)thiophen-2-yl)- 5-(trifluoromethyl)pyrimidin-2-yl)- 1,2,3,4-tetrahydroisoquinolin-6- amine |
| 9 | 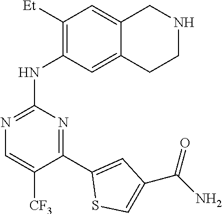 [SMILES: CCc1cc2c(cc1Nc1ncc(C(F)(F)F)c(-c3cc(C(N)=O)cs3)n1)CCNC2] | 5-(2-((7-ethyl-1,2,3,4- tetrahydroisoquinolin-6-yl)amino)- 5-(trifluoromethyl)pyrimidin-4- yl)thiophene-3-carboxamide |
| 10 | 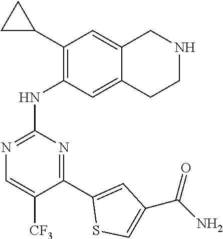 [SMILES: NC(=O)c1csc(-c2nc(Nc3cc4c(cc3C3CC3)CNCC4)ncc2C(F)(F)F)c1] | 5-(2-((7-cyclopropyl-1,2,3,4- tetrahydroisoquinolin-6-yl)amino)- 5-(trifluoromethyl)pyrimidin-4- yl)thiophene-3-carboxamide |
| 11 | 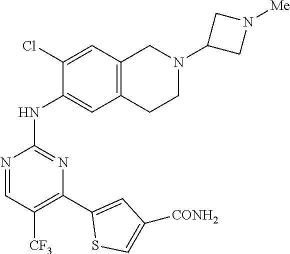 [SMILES: CN1CC(N2CCc3cc(Nc4ncc(C(F)(F)F)c(-c5cc(C(N)=O)cs5)n4)c(Cl)cc3C2)C1] | 5-(2-((7-chloro-2-(1- methylazetidin-3-yl)-1,2,3,4- tetrahydroisoquinolin-6-yl)amino)- 5-(trifluoromethyl)pyrimidin-4- yl)thiophene-3-carboxamide |
| 12 | 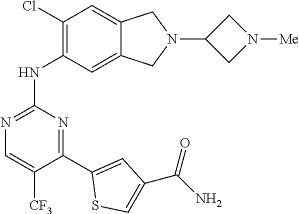 [SMILES: CN1CC(N2Cc3cc(Cl)c(Nc4ncc(C(F)(F)F)c(-c5cc(C(N)=O)cs5)n4)cc3C2)C1] | 5-(2-((6-chloro-2-(1- methylazetidin-3-yl)isoindolin-5- yl)amino)-5- (trifluoromethyl)pyrimidin-4- yl)thiophene-3-carboxamide |
| 13 | 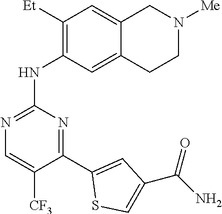 [SMILES: CCc1cc2c(cc1Nc1ncc(C(F)(F)F)c(-c3cc(C(N)=O)cs3)n1)CCN(C)C2] | 5-(2-((7-ethyl-2-methyl-1,2,3,4- tetrahydroisoquinolin-6-yl)amino)- 5-(trifluoromethyl)pyrimidin-4- yl)thiophene-3-carboxamide |
| 14 | 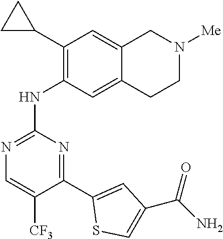 [SMILES: CN1CCc2cc(Nc3ncc(C(F)(F)F)c(-c4cc(C(N)=O)cs4)n3)c(C3CC3)cc2C1] | 5-(2-((7-cyclopropyl-2-methyl- 1,2,3,4-tetrahydroisoquinolin-6- yl)amino)-5- (trifluoromethyl)pyrimidin-4- yl)thiophene-3-carboxamide |
| 15 | 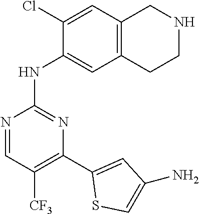 [SMILES: Nc1csc(-c2nc(Nc3cc4c(cc3Cl)CNCC4)ncc2C(F)(F)F)c1] | N-(4-(4-aminothiophen-2-yl)-5- (trifluoromethyl)pyrimidin-2-yl)-7- chloro-1,2,3,4- tetrahydroisoquinolin-6-amine |
| 16 | 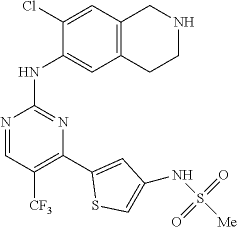 [SMILES: CS(=O)(=O)Nc1csc(-c2nc(Nc3cc4c(cc3Cl)CNCC4)ncc2C(F)(F)F)c1] | N-(5-(2-((7-chloro-1,2,3,4- tetrahydroisoquinolin-6-yl)amino)- 5-(trifluoromethyl)pyrimidin-4- yl)thiophen-3- yl)methanesulfonamide |
| 17 | 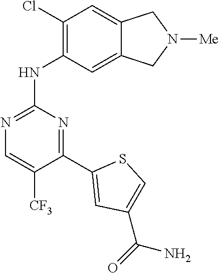 [SMILES: CN1Cc2cc(Cl)c(Nc3ncc(C(F)(F)F)c(-c4cc(C(N)=O)cs4)n3)cc2C1] | 5-(2-((6-Chloro-2- methylisoindolin-5-yl)amino)-5- (trifluoromethyl)pyrimidin-4- yl)thiophene-3-carboxamide |
| 18 | 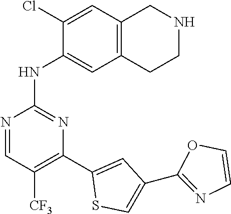 [SMILES: FC(F)(F)c1cnc(Nc2cc3c(cc2Cl)CNCC3)nc1-c1cc(-c2ncco2)cs1] | 7-Chloro-N-(4-(4-(oxazol-2- yl)thiophen-2-yl)-5- (trifluoromethyl)pyrimidin-2-yl)- 1,2,3,4-tetrahydroisoquinolin-6- amine |
| 19 | 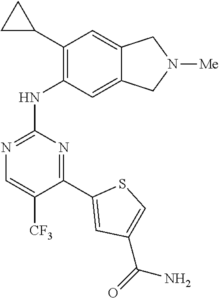 [SMILES: CN1Cc2cc(Nc3ncc(C(F)(F)F)c(-c4cc(C(N)=O)cs4)n3)c(C3CC3)cc2C1] | 5-(2-((6-Cyclopropyl-2- methylisoindolin-5-yl)amino)-5- (trifluoromethyl)pyrimidin-4- yl)thiophene-3-carboxamide |
| 20 | 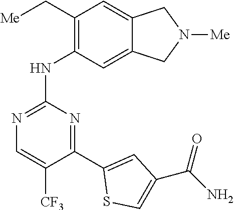 [SMILES: CCc1cc2c(cc1Nc1ncc(C(F)(F)F)c(-c3cc(C(N)=O)cs3)n1)CN(C)C2] | 5-(2-((6-Ethyl-2-methylisoindolin- 5-yl)amino)-5- (trifluoromethyl)pyrimidin-4- yl)thiophene-3-carboxamide |
| 21 | 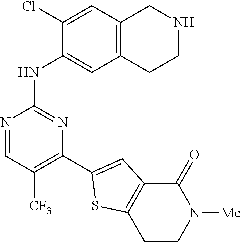 [SMILES: CN1CCc2sc(-c3nc(Nc4cc5c(cc4Cl)CNCC5)ncc3C(F)(F)F)cc2C1=O] | 2-(2-((7-Chloro-1,2,3,4- tetrahydroisoquinolin-6-yl)amino)- 5-(trifluoromethyl)pyrimidin-4-yl)- 5-methyl-6,7-dihydrothieno[3,2- c]pyridin-4(5H)-one |
| 22 | 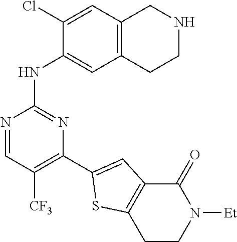 [SMILES: CCN1CCc2sc(-c3nc(Nc4cc5c(cc4Cl)CNCC5)ncc3C(F)(F)F)cc2C1=O] | 2-(2-((7-chloro-1,2,3,4- tetrahydroisoquinolin-6-yl)amino)- 5-(trifluoromethyl)pyrimidin-4-yl)- 5-ethyl-6,7-dihydrothieno[3,2- c]pyridin-4(5H)-one |
| 23 | 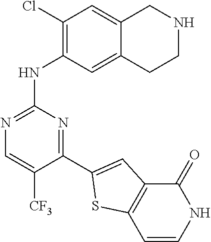 [SMILES: O=c1[nH]ccc2sc(-c3nc(Nc4cc5c(cc4Cl)CNCC5)ncc3C(F)(F)F)cc12] | 2-(2-((7-Chloro-1,2,3,4- tetrahydroisoquinolin-6-yl)amino)- 5-(trifluoromethyl)pyrimidin-4- yl)thieno[3,2-c]pyridin-4(5H)-one |
| 24 | 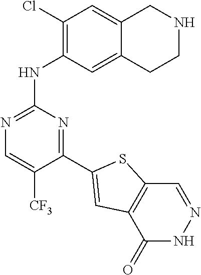 [SMILES: O=c1[nH]ncc2sc(-c3nc(Nc4cc5c(cc4Cl)CNCC5)ncc3C(F)(F)F)cc12] | 2-(2-((7-Chloro-1,2,3,4- tetrahydroisoquinolin-6-yl)amino)- 5-(trifluoromethyl)pyrimidin-4- yl)thieno[2,3-d]pyridazin-4(5H)- one |
| 25 | 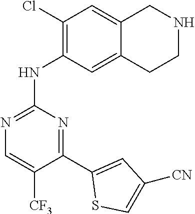 [SMILES: N#Cc1csc(-c2nc(Nc3cc4c(cc3Cl)CNCC4)ncc2C(F)(F)F)c1] | 5-(2-((7-Chloro-1,2,3,4- tetrahydroisoquinolin-6-yl)amino)- 5-(trifluoromethyl)pyrimidin-4- yl)thiophene-3-carbonitrile |
| 26 | 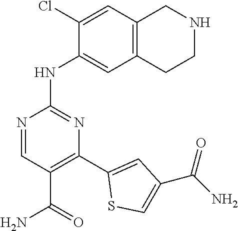 [SMILES: NC(=O)c1csc(-c2nc(Nc3cc4c(cc3Cl)CNCC4)ncc2C(N)=O)c1] | 4-(4-Carbamoylthiophen-2-y1)-2- ((7-chloro-1,2,3,4- tetrahydroisoquinolin-6- yl)amino)pyrimidine-5- carboxamide |
| 27 | 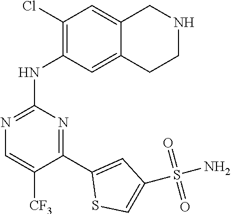 [SMILES: NS(=O)(=O)c1csc(-c2nc(Nc3cc4c(cc3Cl)CNCC4)ncc2C(F)(F)F)c1] | 5-(2-((7-Chloro-1,2,3,4- tetrahydroisoquinolin-6-yl)amino)- 5-(trifluoromethyl)pyrimidin-4- yl)thiophene-3-sulfona |
| 28 | 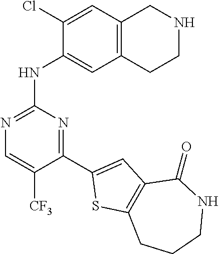 [SMILES: O=C1NCCCc2sc(-c3nc(Nc4cc5c(cc4Cl)CNCC5)ncc3C(F)(F)F)cc21] | 2-(2-((7-Chloro-1,2,3,4- tetrahydroisoquinolin-6-yl)amino)- 5-(trifluoromethyl)pyrimidin-4-yl)- 5,6,7,8-tetrahydro-4H-thieno[3,2- c]azepin-4-one |
| 29 | 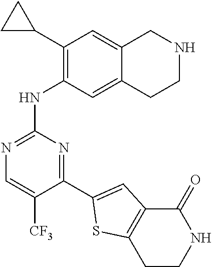 [SMILES: O=C1NCCc2sc(-c3nc(Nc4cc5c(cc4C4CC4)CNCC5)ncc3C(F)(F)F)cc21] | 2-(2-((7-Cyclopropyl-1,2,3,4- tetrahydroisoquinolin-6-yl)amino)- 5-(trifluoromethyl)pyrimidin-4-yl)- 6,7-dihydrothieno[3,2-c]pyridin- 4(5H)-one |
| 30 | 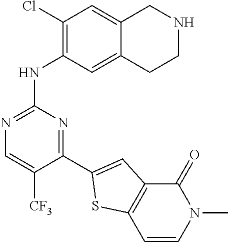 [SMILES: Cn1ccc2sc(-c3nc(Nc4cc5c(cc4Cl)CNCC5)ncc3C(F)(F)F)cc2c1=O] | 2-(2-((7-Chloro-1,2,3,4- tetrahydroisoquinolin-6-yl)amino)- 5-(trifluoromethyl)pyrimidin-4-yl)- 5-methylthieno[3,2-c]pyridin- 4(5H)-one |
| 31 | 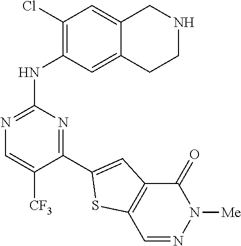 [SMILES: Cn1ncc2sc(-c3nc(Nc4cc5c(cc4Cl)CNCC5)ncc3C(F)(F)F)cc2c1=O] | 2-(2-((7-Chloro-1,2,3,4- tetrahydroisoquinolin-6-yl)amino)- 5-(trifluoromethyl)pyrimidin-4-yl)- 5-methylthieno[2,3-d]pyridazin- 4(5H)-one |
| 32 | 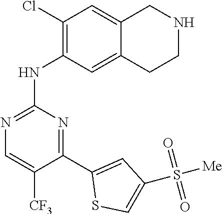 [SMILES: CS(=O)(=O)c1csc(-c2nc(Nc3cc4c(cc3Cl)CNCC4)ncc2C(F)(F)F)c1] | 7-Chloro-N-(4-(4- (methylsulfonyl)thiophen-2-yl)-5- (trifluoromethyl)pyrimidin-2-yl)- 1,2,3,4-tetrahydroisoquinolin-6- amine |
| 33 | 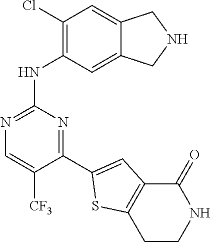 [SMILES: O=C1NCCc2sc(-c3nc(Nc4cc5c(cc4Cl)CNC5)ncc3C(F)(F)F)cc21] | 2-(2-((6-Chloroisoindolin-5- yl)amino)-5- (trifluoromethyl)pyrimidin-4-yl)- 6,7-dihydrothieno[3,2-c]pyridin- 4(5H)-one |
| 34 | 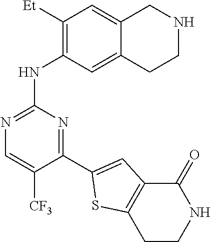 [SMILES: CCc1cc2c(cc1Nc1ncc(C(F)(F)F)c(-c3cc4c(s3)CCNC4=O)n1)CCNC2] | 2-(2-((7-ethyl-1,2,3,4- tetrahydroisoquinolin-6-yl)amino)- 5-(trifluoromethyl)pyrimidin-4-yl)- 6,7-dihydrothieno[3,2-c]pyridin- 4(5H)-one |
| 35 | 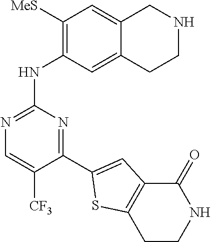 [SMILES: CSc1cc2c(cc1Nc1ncc(C(F)(F)F)c(-c3cc4c(s3)CCNC4=O)n1)CCNC2] | 2-(2-((7-(Methylthio)-1,2,3,4- tetrahydroisoquinolin-6-yl)amino)- 5-(trifluoromethyl)pyrimidin-4-yl)- 6,7-dihydrothieno[3,2-c]pyridin- 4(5H)-one |
| 36 | 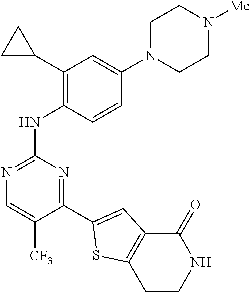 [SMILES: CN1CCN(c2ccc(Nc3ncc(C(F)(F)F)c(-c4cc5c(s4)CCNC5=O)n3)c(C3CC3)c2)CC1] | 2-(2-((2-Cyclopropyl-4-(4- methylpiperazin-1- yl)phenyl)amino)-5- (trifluoromethyl)pyrimidin-4-yl)- 6,7-dihydrothieno[3,2-c]pyridin- 4(5H)-one |
| 37 | 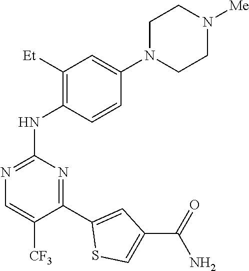 [SMILES: CCc1cc(N2CCN(C)CC2)ccc1Nc1ncc(C(F)(F)F)c(-c2cc(C(N)=O)cs2)n1] | 5-(2-((2-Ethyl-4-(4- methylpiperazin-1- yl)phenyl)amino)-5- (trifluoromethyl)pyrimidin-4- yl)thiophene-3-carboxamide |
| 38 | 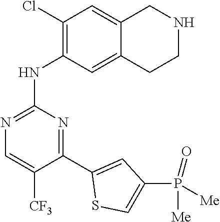 [SMILES: CP(C)(=O)c1csc(-c2nc(Nc3cc4c(cc3Cl)CNCC4)ncc2C(F)(F)F)c1] | (5-(2-((7-Chloro-1,2,3,4- tetrahydroisoquinolin-6-yl)amino)- 5-(trifluoromethyl)pyrimidin-4- yl)thiophen-3- yl)dimethylphosphine oxide |
| 39 | 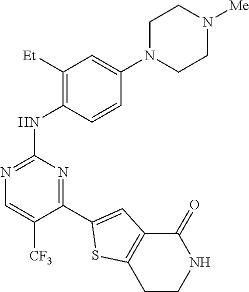 [SMILES: CCc1cc(N2CCN(C)CC2)ccc1Nc1ncc(C(F)(F)F)c(-c2cc3c(s2)CCNC3=O)n1] | 2-(2-((2-ethyl-4-(4- methylpiperazin-1- yl)phenyl)amino)-5- (trifluoromethyl)pyrimidin-4-yl)- 6,7-dihydrothieno[3,2-c]pyridin- 4(5H)-one |
| 40 | 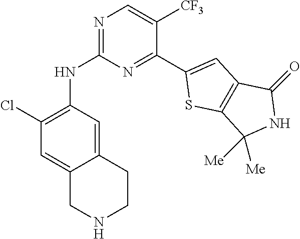 [SMILES: CC1(C)NC(=O)c2cc(-c3nc(Nc4cc5c(cc4Cl)CNCC5)ncc3C(F)(F)F)sc21] | 2-(2-((7-chloro-1,2,3,4- tetrahydroisoquinolin-6-yl)amino)- 5-(trifluoromethyl)pyrimidin-4-yl)- 6,6-dimethyl-5,6-dihydro-4H- thieno[2,3-c]pyrrol-4-one |
| 41 | 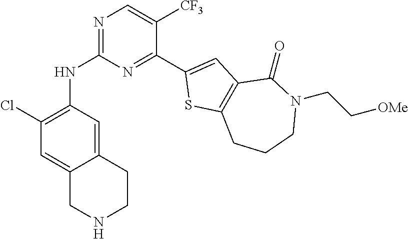 [SMILES: COCCN1CCCc2sc(-c3nc(Nc4cc5c(cc4Cl)CNCC5)ncc3C(F)(F)F)cc2C1=O] | 2-(2-((7-chloro-1,2,3,4- tetrahydroisoquinolin-6-yl)amino)- 5-(trifluoromethyl)pyrimidin-4-yl)- 5-(2-methoxyethyl)-5,6,7,8- tetrahydro-4H-thieno[3,2-c]azepin- 4-one |
| 42 | 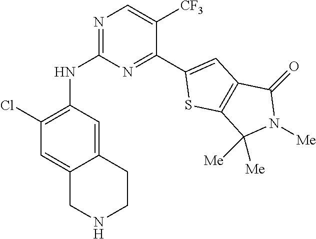 [SMILES: CN1C(=O)c2cc(-c3nc(Nc4cc5c(cc4Cl)CNCC5)ncc3C(F)(F)F)sc2C1(C)C] | 2-(2-((7-chloro-1,2,3,4- tetrahydroisoquinolin-6-yl)amino)- 5-(trifluoromethyl)pyrimidin-4-yl)- 5,6,6-trimethyl-5,6-dihydro-4H- thieno[2,3-c]pyrrol-4-one |
| 43 | 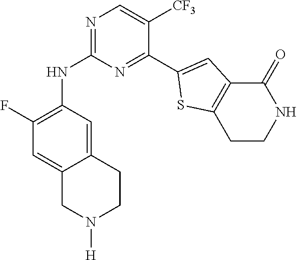 [SMILES: O=C1NCCc2sc(-c3nc(Nc4cc5c(cc4F)CNCC5)ncc3C(F)(F)F)cc21] | 2-(2-((7-fluoro-1,2,3,4- tetrahydroisoquinolin-6-yl)amino)- 5-(trifluoromethyl)pyrimidin-4-yl)- 6,7-dihydrothieno[3,2-c]pyridin- 4(5H)-one |
| 44 | 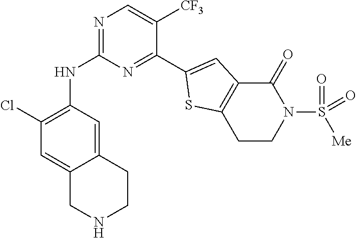 [SMILES: CS(=O)(=O)N1CCc2sc(-c3nc(Nc4cc5c(cc4Cl)CNCC5)ncc3C(F)(F)F)cc2C1=O] | 2-(2-((7-chloro-1,2,3,4- tetrahydroisoquinolin-6-yl)amino)- 5-(trifluoromethyl)pyrimidin-4-yl)- 5-(methylsulfonyl)-6,7- dihydrothieno[3,2-c]pyridin-4(5H)- one |
| 45 | 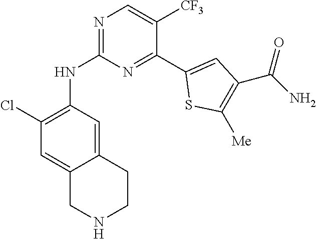 [SMILES: Cc1sc(-c2nc(Nc3cc4c(cc3Cl)CNCC4)ncc2C(F)(F)F)cc1C(N)=O] | 5-(2-((7-chloro-1,2,3,4- tetrahydroisoquinolin-6-yl)amino)- 5-(trifluoromethyl)pyrimidin-4-yl)- 2-methylthiophene-3-carboxamide |
| 46 | 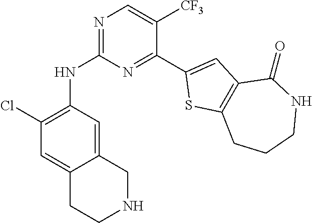 [SMILES: O=C1NCCCc2sc(-c3nc(Nc4cc5c(cc4Cl)CCNC5)ncc3C(F)(F)F)cc21] | 2-(2-((6-chloro-1,2,3,4- tetrahydroisoquinolin-7-yl)amino)- 5-(trifluoromethyl)pyrimidin-4-yl)- 5,6,7,8-tetrahydro-4H-thieno[3,2- c]azepin-4-one |
| 47 | 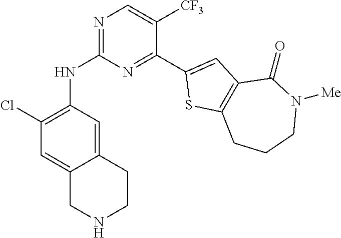 [SMILES: CN1CCCc2sc(-c3nc(Nc4cc5c(cc4Cl)CNCC5)ncc3C(F)(F)F)cc2C1=O] | 2-(2-((7-chloro-1,2,3,4- tetrahydroisoquinolin-6-yl)amino)- 5-(trifluoromethyl)pyrimidin-4-yl)- 5-methyl-5,6,7,8-tetrahydro-4H- thieno[3,2-c]azepin-4-one |
| 48 | 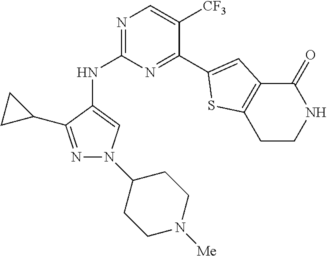 [SMILES: CN1CCC(n2cc(Nc3ncc(C(F)(F)F)c(-c4cc5c(s4)CCNC5=O)n3)c(C3CC3)n2)CC1] | 2-(2-((3-cyclopropyl-1-(1- methylpiperidin-4-yl)-1H-pyrazol- 4-yl)amino)-5- (trifluoromethyl)pyrimidin-4-yl)- 6,7-dihydrothieno[3,2-c]pyridin- 4(5H)-one |
| 49 | 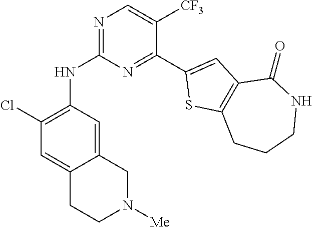 [SMILES: CN1CCc2cc(Cl)c(Nc3ncc(C(F)(F)F)c(-c4cc5c(s4)CCCNC5=O)n3)cc2C1] | 2-(2-((6-chloro-2-methyl-1,2,3,4- tetrahydroisoquinolin-7-yl)amino)- 5-(trifluoromethyl)pyrimidin-4-yl)- 5,6,7,8-tetrahydro-4H-thieno[3,2- c]azepin-4-one |
| 50 | 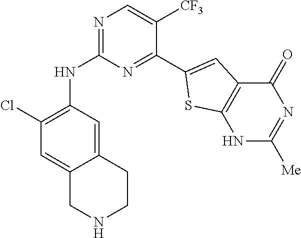 [SMILES: Cc1nc(=O)c2cc(-c3nc(Nc4cc5c(cc4Cl)CNCC5)ncc3C(F)(F)F)sc2[nH]1] | 6-(2-((7-chloro-1,2,3,4- tetrahydroisoquinolin-6-yl)amino)- 5-(trifluoromethyl)pyrimidin-4-yl)- 2-methylthieno[2,3-d]pyrimidin- 4(1H)-one |
| 51 | 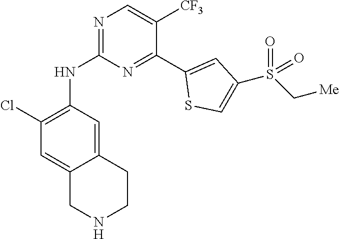 [SMILES: CCS(=O)(=O)c1csc(-c2nc(Nc3cc4c(cc3Cl)CNCC4)ncc2C(F)(F)F)c1] | 7-chloro-N-(4-(4- (ethylsulfonyl)thiophen-2-yl)-5- (trifluoromethyl)pyrimidin-2-yl)- 1,2,3,4-tetrahydroisoquinolin-6- amine |
| 52 | 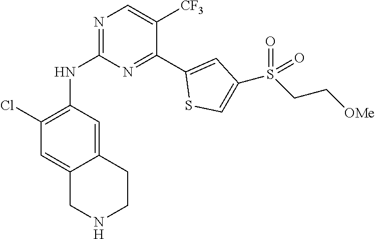 [SMILES: COCCS(=O)(=O)c1csc(-c2nc(Nc3cc4c(cc3Cl)CNCC4)ncc2C(F)(F)F)c1] | 7-chloro-N-(4-(4-((2- methoxyethyl)sulfonyl)thiophen-2- yl)-5-(trifluoromethyl)pyrimidin-2- yl)-1,2,3,4-tetrahydroisoquinolin-6- amine |
| 53 | 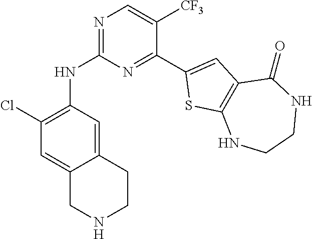 [SMILES: O=C1NCCNc2sc(-c3nc(Nc4cc5c(cc4Cl)CNCC5)ncc3C(F)(F)F)cc21] | 7-(2-((7-chloro-1,2,3,4- tetrahydroisoquinolin-6-yl)amino)- 5-(trifluoromethyl)pyrimidin-4-yl)- 1,2,3,4-tetrahydro-5H-thieno[2,3- e][1,4]diazepin-5-one |
| 54 | 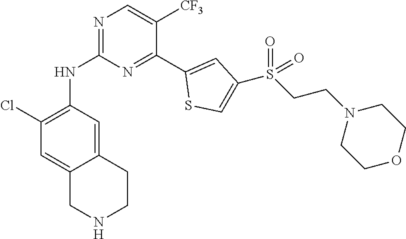 [SMILES: O=S(=O)(CCN1CCOCC1)c1csc(-c2nc(Nc3cc4c(cc3Cl)CNCC4)ncc2C(F)(F)F)c1] | 7-chloro-N-(4-(4-((2- morpholinoethyl)sulfonyl)thiophen- 2-yl)-5-(trifluoromethyl)pyrimidin- 2-yl)-1,2,3,4-tetrahydroisoquinolin- 6-amine |
| 55 | 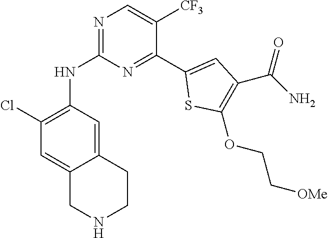 [SMILES: COCCOc1sc(-c2nc(Nc3cc4c(cc3Cl)CNCC4)ncc2C(F)(F)F)cc1C(N)=O] | 5-(2-((7-chloro-1,2,3,4- tetrahydroisoquinolin-6-yl)amino)- 5-(trifluoromethyl)pyrimidin-4-yl)- 2-(2-methoxyethoxy)thiophene-3- carboxamide |
| 56 | 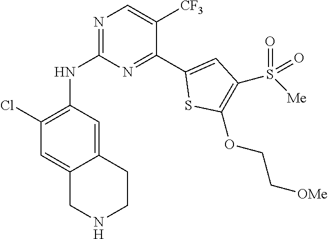 [SMILES: COCCOc1sc(-c2nc(Nc3cc4c(cc3Cl)CNCC4)ncc2C(F)(F)F)cc1S(C)(=O)=O] | 7-chloro-N-(4-(5-(2- methoxyethoxy)-4- (methylsulfonyl)thiophen-2-yl)-5- (trifluoromethyl)pyrimidin-2-yl)- 1,2,3,4-tetrahydroisoquinolin-6- amine |
| 57 | 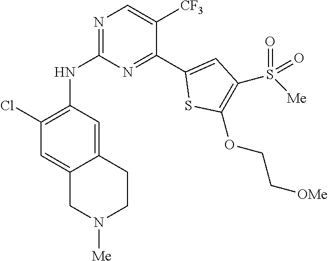 [SMILES: COCCOc1sc(-c2nc(Nc3cc4c(cc3Cl)CN(C)CC4)ncc2C(F)(F)F)cc1S(C)(=O)=O] | 7-chloro-N-(4-(5-(2- methoxyethoxy)-4- (methylsulfonyl)thiophen-2-yl)-5- (trifluoromethyl)pyrimidin-2-yl)-2- methyl-1,2,3,4- tetrahydroisoquinolin-6-amine |
| 58 | 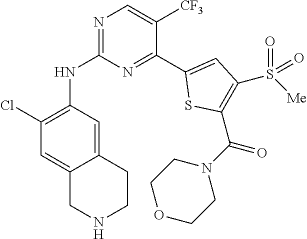 [SMILES: CS(=O)(=O)c1cc(-c2nc(Nc3cc4c(cc3Cl)CNCC4)ncc2C(F)(F)F)sc1C(=O)N1CCOCC1] | (5-(2-((7-chloro-1,2,3,4- tetrahydroisoquinolin-6-yl)amino)- 5-(trifluoromethyl)pyrimidin-4-yl)- 3-(methylsulfonyl)thiophen-2- yl)(morpholino)methanone |
| 59 | 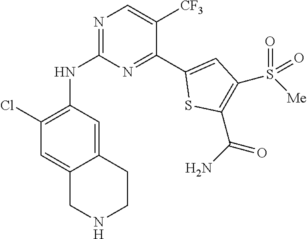 [SMILES: CS(=O)(=O)c1cc(-c2nc(Nc3cc4c(cc3Cl)CNCC4)ncc2C(F)(F)F)sc1C(N)=O] | 5-(2-((7-chloro-1,2,3,4- tetrahydroisoquinolin-6-yl)amino)- 5-(trifluoromethyl)pyrimidin-4-yl)- 3-(methylsulfonyl)thiophene-2- carboxamide |
| 60 | 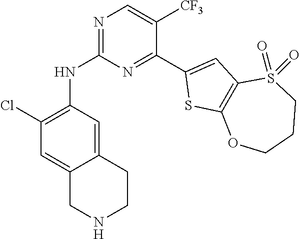 [SMILES: O=S1(=O)CCCOc2sc(-c3nc(Nc4cc5c(cc4Cl)CNCC5)ncc3C(F)(F)F)cc21] | 2-(2-((7-chloro-1,2,3,4- tetrahydroisoquinolin-6-yl)amino)- 5-(trifluoromethyl)pyrimidin-4-yl)- 6,7-dihydro-5H-thieno[2,3- b][1,4]oxathiepine 4,4-dioxide |
| 61 | 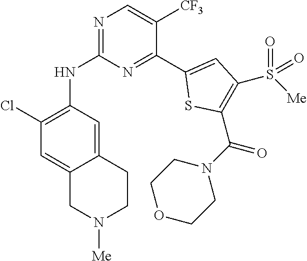 [SMILES: CN1CCc2cc(Nc3ncc(C(F)(F)F)c(-c4cc(S(C)(=O)=O)c(C(=O)N5CCOCC5)s4)n3)c(Cl)cc2C1] | (5-(2-((7-chloro-2-methyl-1,2,3,4- tetrahydroisoquinolin-6-yl)amino)- 5-(trifluoromethyl)pyrimidin-4-yl)- 3-(methylsulfonyl)thiophen-2- yl)(morpholino)methanone |
| 62 | 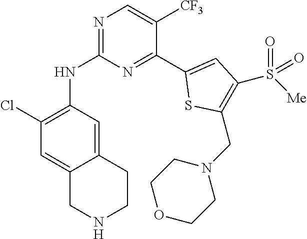 [SMILES: CS(=O)(=O)c1cc(-c2nc(Nc3cc4c(cc3Cl)CNCC4)ncc2C(F)(F)F)sc1CN1CCOCC1] | 7-chloro-N-(4-(4-(methylsulfonyl)- 5-(morpholinomethyl)thiophen-2- yl)-5-(trifluoromethyl)pyrimidin-2- yl)-1,2,3,4-tetrahydroisoquinolin-6- amine |
| 63 | 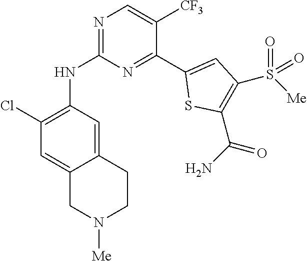 [SMILES: CN1CCc2cc(Nc3ncc(C(F)(F)F)c(-c4cc(S(C)(=O)=O)c(C(N)=O)s4)n3)c(Cl)cc2C1] | 5-(2-((7-chloro-2-methyl-1,2,3,4- tetrahydroisoquinolin-6-yl)amino)- 5-(trifluoromethyl)pyrimidin-4-yl)- 3-(methylsulfonyl)thiophene-2- carboxamide |
| 64 | 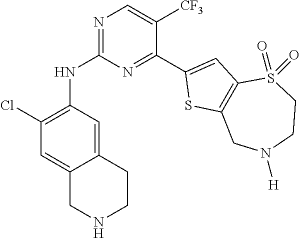 [SMILES: O=S1(=O)CCNCc2sc(-c3nc(Nc4cc5c(cc4Cl)CNCC5)ncc3C(F)(F)F)cc21] | 7-(2-((7-chloro-1,2,3,4- tetrahydroisoquinolin-6-yl)amino)- 5-(trifluoromethyl)pyrimidin-4-yl)- 2,3,4,5-tetrahydrothieno[2,3- f][1,4]thiazepine 1,1-dioxide |
| 65 | 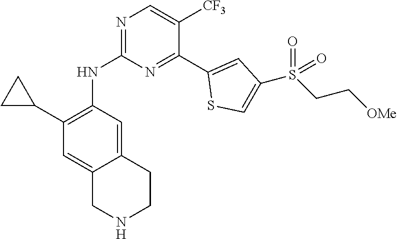 [SMILES: COCCS(=O)(=O)c1csc(-c2nc(Nc3cc4c(cc3C3CC3)CNCC4)ncc2C(F)(F)F)c1] | 7-cyclopropyl-N-(4-(4-((2- methoxyethyl)sulfonyl)thiophen-2- yl)-5-(trifluoromethyl)pyrimidin-2- yl)-1,2,3,4-tetrahydroisoquinolin-6- amine |
| 66 | 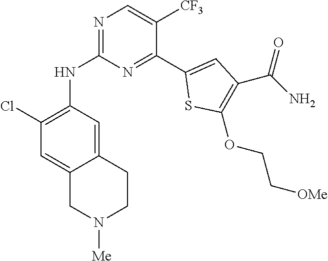 [SMILES: COCCOc1sc(-c2nc(Nc3cc4c(cc3Cl)CN(C)CC4)ncc2C(F)(F)F)cc1C(N)=O] | 5-(2-((7-chloro-2-methyl-1,2,3,4- tetrahydroisoquinolin-6-yl)amino)- 5-(trifluoromethyl)pyrimidin-4-yl)- 2-(2-methoxyethoxy)thiophene-3- carboxamide |
| 67 | 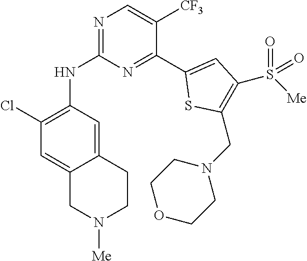 [SMILES: CN1CCc2cc(Nc3ncc(C(F)(F)F)c(-c4cc(S(C)(=O)=O)c(CN5CCOCC5)s4)n3)c(Cl)cc2C1] | 7-chloro-2-methyl-N-(4-(4- (methylsulfonyl)-5- (morpholinomethyl)thiophen-2-yl)- 5-(trifluoromethyl)pyrimidin-2-yl)- 1,2,3,4-tetrahydroisoquinolin-6- amine |
| 68 | 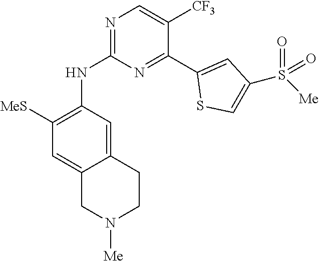 [SMILES: CSc1cc2c(cc1Nc1ncc(C(F)(F)F)c(-c3cc(S(C)(=O)=O)cs3)n1)CCN(C)C2] | 2-methyl-N-(4-(4- (methylsulfony1)thiophen-2-yl)-5- (trifluoromethyl)pyrimidin-2-yl)-7- (methylthio)-1,2,3,4- tetrahydroisoquinolin-6-amine |
| 69 | 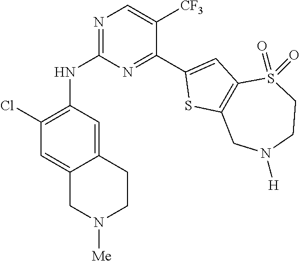 [SMILES: CN1CCc2cc(Nc3ncc(C(F)(F)F)c(-c4cc5c(s4)CNCCS5(=O)=O)n3)c(Cl)cc2C1] | 7-(2-((7-chloro-2-methyl-1,2,3,4- tetrahydroisoquinolin-6-yl)amino)- 5-(trifluoromethyl)pyrimidin-4-yl)- 2,3,4,5-tetrahydrothieno[2,3- f][1,4]thiazepine 1,1-dioxide |
| 71 | 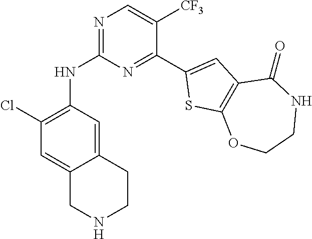 [SMILES: O=C1NCCOc2sc(-c3nc(Nc4cc5c(cc4Cl)CNCC5)ncc3C(F)(F)F)cc21] | 7-(2-((7-chloro-1,2,3,4- tetrahydroisoquinolin-6-yl)amino)- 5-(trifluoromethyl)pyrimidin-4-yl)- 3,4-dihydrothieno[3,2- f][1,4]oxazepin-5(2H)-one |
| 72 | 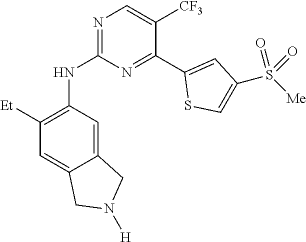 [SMILES: CCc1cc2c(cc1Nc1ncc(C(F)(F)F)c(-c3cc(S(C)(=O)=O)cs3)n1)CNC2] | 6-ethyl-N-(4-(4- (methylsulfony1)thiophen-2-yl)-5- (trifluoromethyl)pyrimidin-2- yl)isoindolin-5-amine |
| 73 | 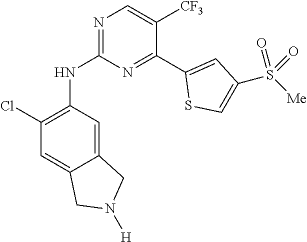 [SMILES: CS(=O)(=O)c1csc(-c2nc(Nc3cc4c(cc3Cl)CNC4)ncc2C(F)(F)F)c1] | 6-chloro-N-(4-(4- (methylsulfonyl)thiophen-2-yl)-5- (trifluoromethyl)pyrimidin-2- yl)isoindolin-5-amine |
| 75 | 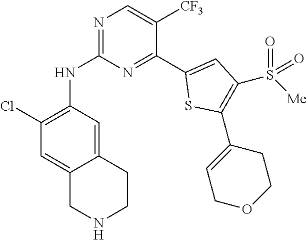 [SMILES: CS(=O)(=O)c1cc(-c2nc(Nc3cc4c(cc3Cl)CNCC4)ncc2C(F)(F)F)sc1C1=CCOCC1] | 7-chloro-N-(4-(5-(3,6-dihydro-2H- pyran-4-yl)-4- (methylsulfonyl)thiophen-2-yl)-5- (trifluoromethyl)pyrimidin-2-yl)- 1,2,3,4-tetrahydroisoquinolin-6- amine |
| 76 | 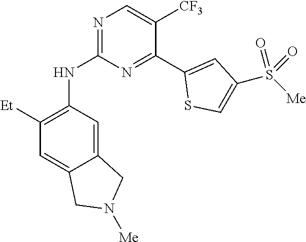 [SMILES: CCc1cc2c(cc1Nc1ncc(C(F)(F)F)c(-c3cc(S(C)(=O)=O)cs3)n1)CN(C)C2] | 6-ethyl-2-methyl-N-(4-(4- (methylsulfonyl)thiophen-2-yl)-5- (trifluoromethyl)pyrimidin-2- yl)isoindolin-5-amine |
| 77 | 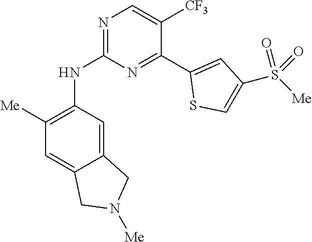 [SMILES: Cc1cc2c(cc1Nc1ncc(C(F)(F)F)c(-c3cc(S(C)(=O)=O)cs3)n1)CN(C)C2] | 6-chloro-2-methyl-N-(4-(4- (methylsulfonyl)thiophen-2-yl)-5- (trifluoromethyl)pyrimidin-2- yl)isoindolin-5-amine |
| 78 | 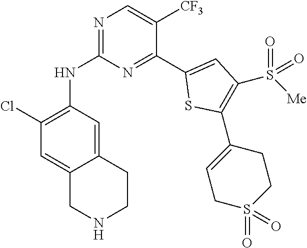 [SMILES: CS(=O)(=O)c1cc(-c2nc(Nc3cc4c(cc3Cl)CNCC4)ncc2C(F)(F)F)sc1C1=CCS(=O)(=O)CC1] | 4-(5-(2-((7-chloro-1,2,3,4- tetrahydroisoquinolin-6-yl)amino)- 5-(trifluoromethyl)pyrimidin-4-yl)- 3-(methylsulfonyl)thiophen-2-yl)- 3,6-dihydro-2H-thiopyran 1,1- dioxide |
| 79 | 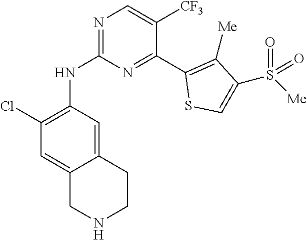 [SMILES: Cc1c(S(C)(=O)=O)csc1-c1nc(Nc2cc3c(cc2Cl)CNCC3)ncc1C(F)(F)F] | 7-chloro-N-(4-(3-methyl-4- (methylsulfonyl)thiophen-2-yl)-5- (trifluoromethyl)pyrimidin-2-yl)- 1,2,3,4-tetrahydroisoquinolin-6- amine |
| 80 | 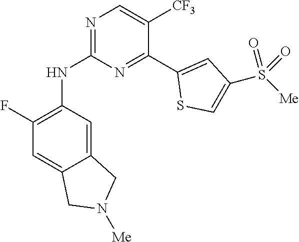 [SMILES: CN1Cc2cc(F)c(Nc3ncc(C(F)(F)F)c(-c4cc(S(C)(=O)=O)cs4)n3)cc2C1] | 6-fluoro-2-methyl-N-(4-(4- (methylsulfonyl)thiophen-2-yl)-5- (trifluoromethyl)pyrimidin-2- yl)isoindolin-5-amine |
| 81 | 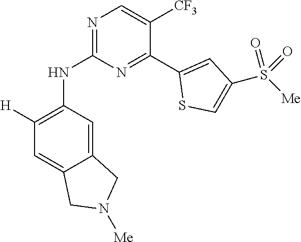 [SMILES: CN1Cc2ccc(Nc3ncc(C(F)(F)F)c(-c4cc(S(C)(=O)=O)cs4)n3)cc2C1] | 2-methyl-N-(4-(4- (methylsulfonyl)thiophen-2-yl)-5- (trifluoromethyl)pyrimidin-2- yl)isoindolin-5-amine |
| 82 | 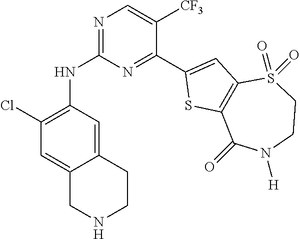 [SMILES: O=C1NCCS(=O)(=O)c2cc(-c3nc(Nc4cc5c(cc4Cl)CNCC5)ncc3C(F)(F)F)sc21] | 7-(2-((7-chloro-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 83 | 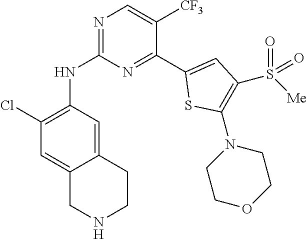 [SMILES: CS(=O)(=O)c1cc(-c2nc(Nc3cc4c(cc3Cl)CNCC4)ncc2C(F)(F)F)sc1N1CCOCC1] | 7-chloro-N-(4-(4-(methylsulfonyl)- 5-morpholinothiophen-2-yl)-5- (trifluoromethyl)pyrimidin-2-yl)- 1,2,3,4-tetrahydroisoquinolin-6- amine |
| 84 | 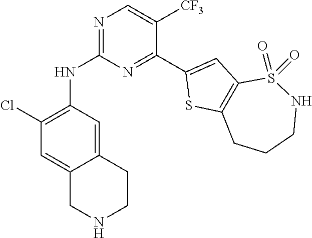 [SMILES: O=S1(=O)NCCCc2sc(-c3nc(Nc4cc5c(cc4Cl)CNCC5)ncc3C(F)(F)F)cc21] | 7-(2-((7-chloro-1,2,3,4- tetrahydroisoquinolin-6-yl)amino)- 5-(trifluoromethyl)pyrimidin-4-yl)- 2,3,4,5-tetrahydrothieno[2,3- f][1,2]thiazepine 1,1-dioxide |
| 85 | 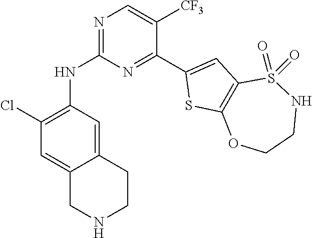 [SMILES: O=S1(=O)NCCOc2sc(-c3nc(Nc4cc5c(cc4Cl)CNCC5)ncc3C(F)(F)F)cc21] | 7-(2-((7-chloro-1,2,3,4- tetrahydroisoquinolin-6-yl)amino)- 5-(trifluoromethyl)pyrimidin-4-yl)- 3,4-dihydro-2H-thieno[2,3- b][1,4,5]oxathiazepine 1,1-dioxide |
| 86 | 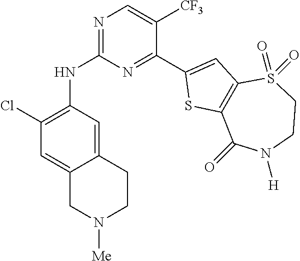 [SMILES: CN1CCc2cc(Nc3ncc(C(F)(F)F)c(-c4cc5c(s4)C(=O)NCCS5(=O)=O)n3)c(Cl)cc2C1] | 7-(2-((7-chloro-2-methyl-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 87 | 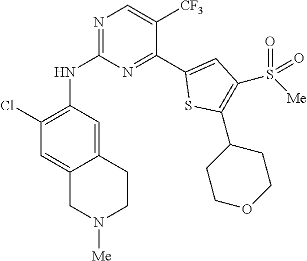 [SMILES: CN1CCc2cc(Nc3ncc(C(F)(F)F)c(-c4cc(S(C)(=O)=O)c(C5CCOCC5)s4)n3)c(Cl)cc2C1] | 7-chloro-2-methyl-N-(4-(4- (methylsulfonyl)-5-(tetrahydro-2H- pyran-4-yl)thiophen-2-yl)-5- (trifluoromethyl)pyrimidin-2-yl)- 1,2,3,4-tetrahydroisoquinolin-6- amine |
| 88 | 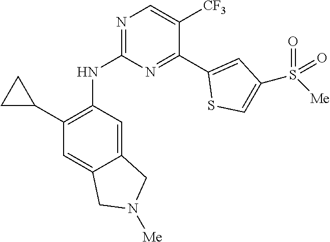 [SMILES: CN1Cc2cc(Nc3ncc(C(F)(F)F)c(-c4cc(S(C)(=O)=O)cs4)n3)c(C3CC3)cc2C1] | 6-cyclopropyl-2-methyl-N-(4-(4- (methylsulfonyl)thiophen-2-yl)-5- (trifluoromethyl)pyrimidin-2- yl)isoindolin-5-amine |
| 89 | 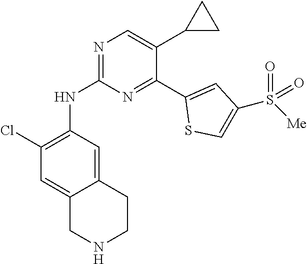 [SMILES: CS(=O)(=O)c1csc(-c2nc(Nc3cc4c(cc3Cl)CNCC4)ncc2C2CC2)c1] | 7-chloro-N-(5-cyclopropyl-4-(4- (methylsulfonyl)thiophen-2- yl)pyrimidin-2-yl)-1,2,3,4- tetrahydroisoquinolin-6-amine |
| 90 | 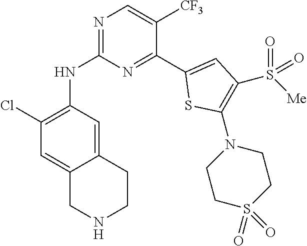 [SMILES: CS(=O)(=O)c1cc(-c2nc(Nc3cc4c(cc3Cl)CNCC4)ncc2C(F)(F)F)sc1N1CCS(=O)(=O)CC1] | 4-(5-(2-((7-chloro-1,2,3,4- tetrahydroisoquinolin-6-yl)amino)- 5-(trifluoromethyl)pyrimidin-4-yl)- 3-(methylsulfonyl)thiophen-2- yl)thiomorpholine 1,1-dioxide |
| 91 | 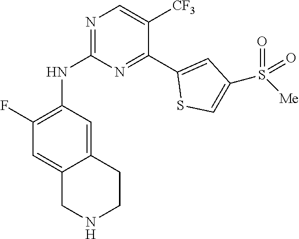 [SMILES: CS(=O)(=O)c1csc(-c2nc(Nc3cc4c(cc3F)CNCC4)ncc2C(F)(F)F)c1] | 7-fluoro-N-(4-(4- (methylsulfonyl)thiophen-2-yl)-5- (trifluoromethyl)pyrimidin-2-yl)- 1,2,3,4-tetrahydroisoquinolin-6- amine |
| 92 | 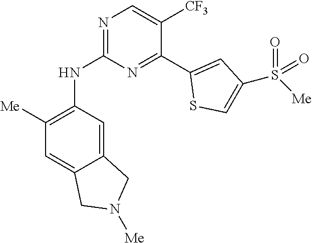 [SMILES: Cc1cc2c(cc1Nc1ncc(C(F)(F)F)c(-c3cc(S(C)(=O)=O)cs3)n1)CN(C)C2] | 2,6-dimethyl-N-(4-(4- (methylsulfonyl)thiophen-2-yl)-5- (trifluoromethyl)pyrimidin-2- yl)isoindolin-5-amine |
| 93 | 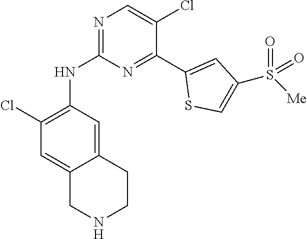 [SMILES: CS(=O)(=O)c1csc(-c2nc(Nc3cc4c(cc3Cl)CNCC4)ncc2Cl)c1] | 7-chloro-N-(5-chloro-4-(4- (methylsulfonyl)thiophen-2- yl)pyrimidin-2-yl)-1,2,3,4- tetrahydroisoquinolin-6-amine |
| 94 | 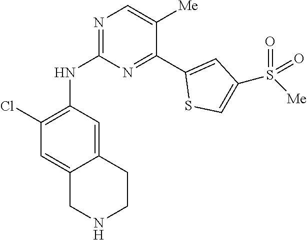 [SMILES: Cc1cnc(Nc2cc3c(cc2Cl)CNCC3)nc1-c1cc(S(C)(=O)=O)cs1] | 7-chloro-N-(5-methyl-4-(4- (methylsulfonyl)thiophen-2- yl)pyrimidin-2-yl)-1,2,3,4- tetrahydroisoquinolin-6-amine |
| 95 | 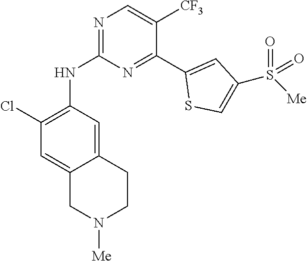 [SMILES: CN1CCc2cc(Nc3ncc(C(F)(F)F)c(-c4cc(S(C)(=O)=O)cs4)n3)c(Cl)cc2C1] | 7-chloro-2-methyl-N-(4-(4- (methylsulfonyl)thiophen-2-yl)-5- (trifluoromethyl)pyrimidin-2-yl)- 1,2,3,4-tetrahydroisoquinolin-6- amine |
| 96 | 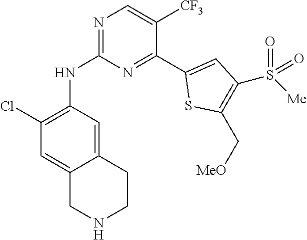 [SMILES: COCc1sc(-c2nc(Nc3cc4c(cc3Cl)CNCC4)ncc2C(F)(F)F)cc1S(C)(=O)=O] | 7-chloro-N-(4-(5-(methoxymethyl)- 4-(methylsulfonyl)thiophen-2-yl)- 5-(trifluoromethyl)pyrimidin-2-yl)- 1,2,3,4-tetrahydroisoquinolin-6- amine |
| 97 | 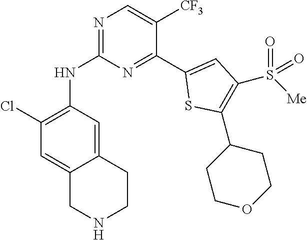 [SMILES: CS(=O)(=O)c1cc(-c2nc(Nc3cc4c(cc3Cl)CNCC4)ncc2C(F)(F)F)sc1C1CCOCC1] | 7-chloro-N-(4-(4-(methylsulfonyl)- 5-(tetrahydro-2H-pyran-4- yl)thiophen-2-yl)-5- (trifluoromethyl)pyrimidin-2-yl)- 1,2,3,4-tetrahydroisoquinolin-6- amine |
| 98 | 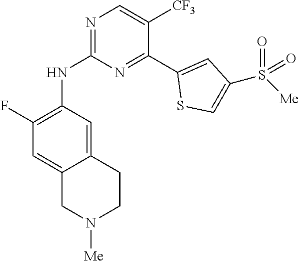 [SMILES: CN1CCc2cc(Nc3ncc(C(F)(F)F)c(-c4cc(S(C)(=O)=O)cs4)n3)c(F)cc2C1] | 7-fluoro-2-methyl-N-(4-(4- (methylsulfonyl)thiophen-2-yl)-5- (trifluoromethyl)pyrimidin-2-yl)- 1,2,3,4-tetrahydroisoquinolin-6- amine |
| 99 | 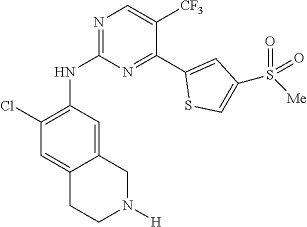 [SMILES: CS(=O)(=O)c1csc(-c2nc(Nc3cc4c(cc3Cl)CCNC4)ncc2C(F)(F)F)c1] | 6-chloro-N-(4-(4- (methylsulfonyl)thiophen-2-yl)-5- (trifluoromethyl)pyrimidin-2-yl)- 1,2,3,4-tetrahydroisoquinolin-7- amine |
| 100 | 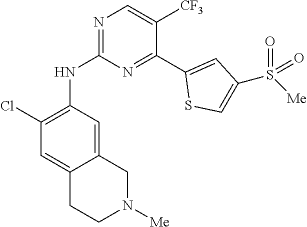 [SMILES: CN1CCc2cc(Cl)c(Nc3ncc(C(F)(F)F)c(-c4cc(S(C)(=O)=O)cs4)n3)cc2C1] | 6-chloro-2-methyl-N-(4-(4- (methylsulfonyl)thiophen-2-yl)-5- (trifluoromethyl)pyrimidin-2-yl)- 1,2,3,4-tetrahydroisoquinolin-7- amine |
| 101 | 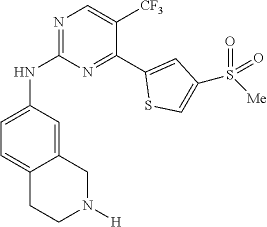 [SMILES: CS(=O)(=O)c1csc(-c2nc(Nc3ccc4c(c3)CNCC4)ncc2C(F)(F)F)c1] | N-(4-(4-(methylsulfonyl)thiophen- 2-y1)-5-(trifluoromethyl)pyrimidin- 2-yl)-1,2,3,4-tetrahydroisoquinolin- 7-amine |
| 102 | 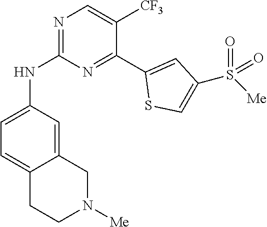 [SMILES: CN1CCc2ccc(Nc3ncc(C(F)(F)F)c(-c4cc(S(C)(=O)=O)cs4)n3)cc2C1] | 2-methyl-N-(4-(4- (methylsulfonyl)thiophen-2-yl)-5- (trifluoromethyl)pyrimidin-2-yl)- 1,2,3,4-tetrahydroisoquinolin-7- amine |
| 103 | 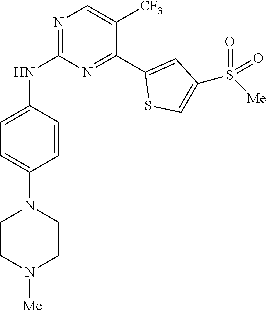 [SMILES: CN1CCN(c2ccc(Nc3ncc(C(F)(F)F)c(-c4cc(S(C)(=O)=O)cs4)n3)cc2)CC1] | N-(4-(4-methylpiperazin-1- yl)phenyl)-4-(4- (methylsulfonyl)thiophen-2-yl)-5- (trifluoromethyl)pyrimidin-2-amine |
| 104 | 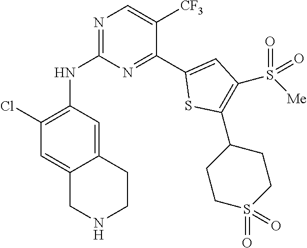 [SMILES: CS(=O)(=O)c1cc(-c2nc(Nc3cc4c(cc3Cl)CNCC4)ncc2C(F)(F)F)sc1C1CCS(=O)(=O)CC1] | 4-(5-(2-((7-chloro-1,2,3,4- tetrahydroisoquinolin-6-yl)amino)- 5-(trifluoromethyl)pyrimidin-4-yl)- 3-(methylsulfonyl)thiophen-2- yl)tetrahydro-2H-thiopyran 1,1- dioxide |
| 105 | 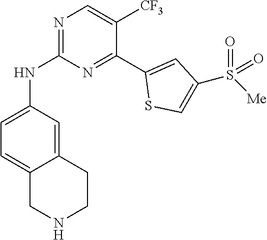 [SMILES: CS(=O)(=O)c1csc(-c2nc(Nc3ccc4c(c3)CCNC4)ncc2C(F)(F)F)c1] | N-(4-(4-(methylsulfonyl)thiophen- 2-yl)-5-(trifluoromethyl)pyrimidin- 2-yl)-1,2,3,4-tetrahydroisoquinolin- 6-amine |
| 106 | 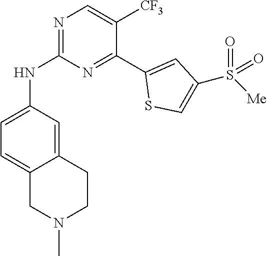 [SMILES: CN1CCc2cc(Nc3ncc(C(F)(F)F)c(-c4cc(S(C)(=O)=O)cs4)n3)ccc2C1] | 2-methyl-N-[4-(4-methylsulfonyl- 2-thienyl)-5- (trifluoromethyl)pyrimidin-2-yl]- 3,4-dihydro-1H-isoquinolin-6- amine |
| 107 | 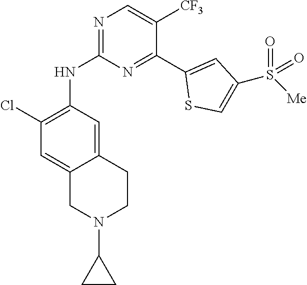 [SMILES: CS(=O)(=O)c1csc(-c2nc(Nc3cc4c(cc3Cl)CN(C3CC3)CC4)ncc2C(F)(F)F)c1] | 7-chloro-2-cyclopropyl-N-[4-(4- methylsulfonyl-2-thienyl)-5- (trifluoromethyl)pyrimidin-2-yl]- 3,4-dihydro-1H-isoquinolin-6- amine |
| 108 | 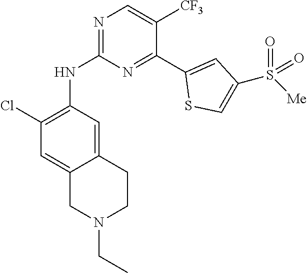 [SMILES: CCN1CCc2cc(Nc3ncc(C(F)(F)F)c(-c4cc(S(C)(=O)=O)cs4)n3)c(Cl)cc2C1] | 7-chloro-N-[4-(4-methylsulfonyl-2- thienyl)-5- (trifluoromethyl)pyrimidin-2-yl]-2- (2,2,2-trifluoroethyl)-3,4-dihydro- 1H-isoquinolin-6-amine |
| 109 | 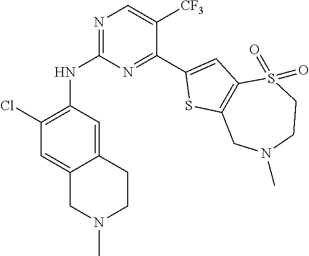 [SMILES: CN1CCc2cc(Nc3ncc(C(F)(F)F)c(-c4cc5c(s4)CN(C)CCS5(=O)=O)n3)c(Cl)cc2C1] | 7-chloro-2-methyl-N-[4-(4-methyl- 1,1-dioxo-3,5-dihydro-2H- thieno[2,3-f][1,4]thiazepin-7-yl)-5- (trifluoromethyl)pyrimidin-2-yl]- 3,4-dihydro-1H-isoquinolin-6- amine |
| 110 | 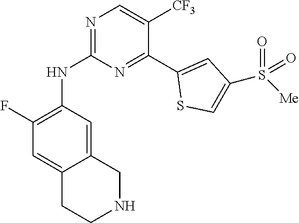 [SMILES: CS(=O)(=O)c1csc(-c2nc(Nc3cc4c(cc3F)CCNC4)ncc2C(F)(F)F)c1] | 6-fluoro-N-[4-(4-methylsulfonyl-2- thienyl)-5- (trifluoromethyl)pyrimidin-2-yl]- 1,2,3,4-tetrahydroisoquinolin-7- amine |
| 111 | 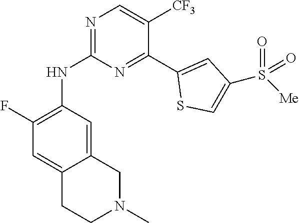 [SMILES: CN1CCc2cc(F)c(Nc3ncc(C(F)(F)F)c(-c4cc(S(C)(=O)=O)cs4)n3)cc2C1] | 6-fluoro-2-methyl-N-[4-(4- methylsulfonyl-2-thienyl)-5- (trifluoromethyl)pyrimidin-2-yl]- 3,4-dihydro-1H-isoquinolin-7- amine |
| 112 | 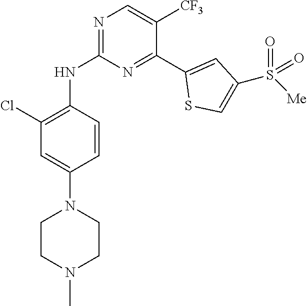 [SMILES: CN1CCN(c2ccc(Nc3ncc(C(F)(F)F)c(-c4cc(S(C)(=O)=O)cs4)n3)c(Cl)c2)CC1] | N-[2-chloro-4-(4-methylpiperazin- 1-yl)phenyl]-4-(4-methylsulfonyl- 2-thienyl)-5- (trifluoromethyl)pyrimidin-2-amine |
| 113 | 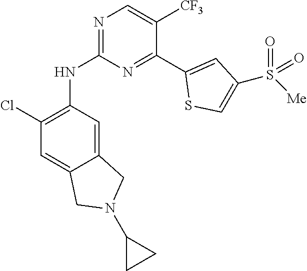 [SMILES: CS(=O)(=O)c1csc(-c2nc(Nc3cc4c(cc3Cl)CN(C3CC3)C4)ncc2C(F)(F)F)c1] | 6-chloro-2-cyclopropyl-N-[4-(4- methylsulfonyl-2-thienyl)-5- (trifluoromethyl)pyrimidin-2- yl]isoindolin-5-amine |
| 114 | 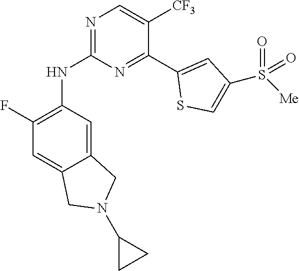 [SMILES: CS(=O)(=O)c1csc(-c2nc(Nc3cc4c(cc3F)CN(C3CC3)C4)ncc2C(F)(F)F)c1] | 2-cyclopropyl-6-fluoro-N-[4-(4- methylsulfonyl-2-thienyl)-5- (trifluoromethyl)pyrimidin-2- yl]isoindolin-5-amine |
| 115 | 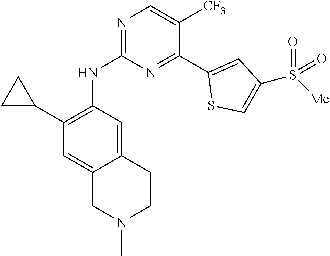 [SMILES: CN1CCc2cc(Nc3ncc(C(F)(F)F)c(-c4cc(S(C)(=O)=O)cs4)n3)c(C3CC3)cc2C1] | 7-cyclopropyl-2-methyl-N-[4-(4- methylsulfonyl-2-thienyl)-5- (trifluoromethyl)pyrimidin-2-yl]- 3,4-dihydro-1H-isoquinolin-6- amine |
| 116 | 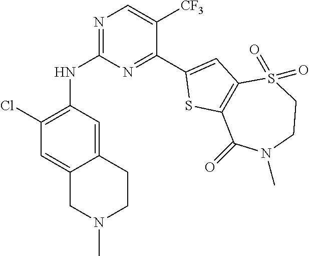 [SMILES: CN1CCc2cc(Nc3ncc(C(F)(F)F)c(-c4cc5c(s4)C(=O)N(C)CCS5(=O)=O)n3)c(Cl)cc2C1] | 7-[2-[(7-chloro-2-methyl-3,4- dihydro-1H-isoquinolin-6- yl)amino]-5- (trifluoromethyl)pyrimidin-4-yl]-4- methyl-1,1-dioxo-2,3- dihydrothieno[2,3-f][1,4]thiazepin- 5-one |
| 117 | 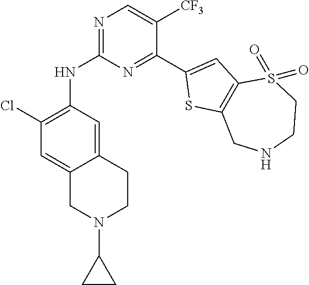 [SMILES: O=S1(=O)CCNCc2sc(-c3nc(Nc4cc5c(cc4Cl)CN(C4CC4)CC5)ncc3C(F)(F)F)cc21] | 7-chloro-2-cyclopropyl-N-[4-(1,1- dioxo-2,3,4,5-tetrahydrothieno[2,3- f][1,4]thiazepin-7-yl)-5- (trifluoromethyl)pyrimidin-2-yl]- 3,4-dihydro-1H-isoquinolin-6- amine |
| 118 | 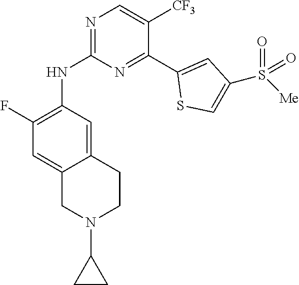 [SMILES: CS(=O)(=O)c1csc(-c2nc(Nc3cc4c(cc3F)CN(C3CC3)CC4)ncc2C(F)(F)F)c1] | 2-cyclopropyl-7-fluoro-N-[4-(4- methylsulfonyl-2-thienyl)-5- (trifluoromethyl)pyrimidin-2-yl]- 3,4-dihydro-1H-isoquinolin-6- amine |
| 119 | 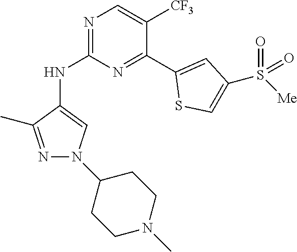 [SMILES: Cc1nn(C2CCN(C)CC2)cc1Nc1ncc(C(F)(F)F)c(-c2cc(S(C)(=O)=O)cs2)n1] | N-[3-methyl-1-(1-methyl-4- piperidyl)pyrazol-4-yl]-4-(4- methylsulfonyl-2-thienyl)-5- (trifluoromethyl)pyrimidin-2-amine |
| 120 | 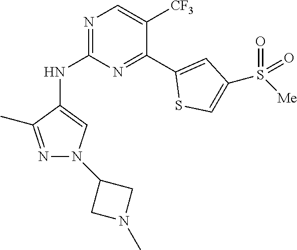 [SMILES: Cc1nn(C2CN(C)C2)cc1Nc1ncc(C(F)(F)F)c(-c2cc(S(C)(=O)=O)cs2)n1] | N-[3-methyl-1-(1-methylazetidin-3- yl)pyrazol-4-yl]-4-(4- methylsulfonyl-2-thienyl)-5- (trifluoromethyl)pyrimidin-2-amine |
| 121 | 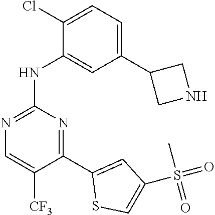 [SMILES: CS(=O)(=O)c1csc(-c2nc(Nc3cc(C4CNC4)ccc3Cl)ncc2C(F)(F)F)c1] | N-(2-chloro-5-(1-methylazetidin-3- yl)phenyl)-4-(4- (methylsulfonyl)thiophen-2-yl)-5- (trifluoromethyl)pyrimidin-2-amine |
| 122 | 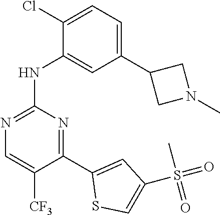 [SMILES: CN1CC(c2ccc(Cl)c(Nc3ncc(C(F)(F)F)c(-c4cc(S(C)(=O)=O)cs4)n3)c2)C1] | N-(2-chloro-5-(1-methylazetidin-3- yl)phenyl)-4-(4- (methylsulfonyl)thiophen-2-yl)-5- (trifluoromethyl)pyrimidin-2-amine |
| 123 | 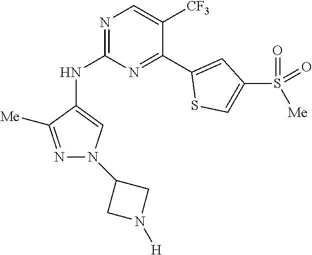 [SMILES: Cc1nn(C2CNC2)cc1Nc1ncc(C(F)(F)F)c(-c2cc(S(C)(=O)=O)cs2)n1] | N-(1-(azetidin-3-yl)-3-methyl-1H- pyrazol-4-yl)-4-(4- (methylsulfonyl)thiophen-2-yl)-5- (trifluoromethyl)pyrimidin-2-amine |
| 124 | 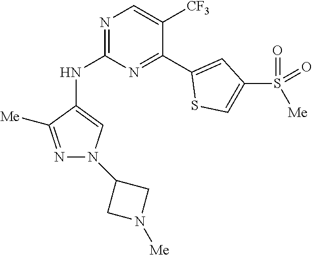 [SMILES: Cc1nn(C2CN(C)C2)cc1Nc1ncc(C(F)(F)F)c(-c2cc(S(C)(=O)=O)cs2)n1] | N-(3-methyl-1-(1-methylazetidin-3- yl)-1H-pyrazol-4-yl)-4-(4- (methylsulfonyl)thiophen-2-yl)-5- (trifluoromethyl)pyrimidin-2-amine |
| 125 | 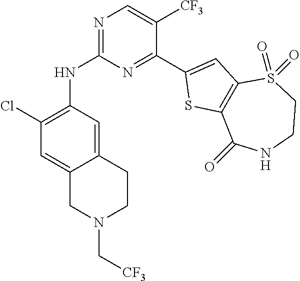 [SMILES: O=C1NCCS(=O)(=O)c2cc(-c3nc(Nc4cc5c(cc4Cl)CN(CC(F)(F)F)CC5)ncc3C(F)(F)F)sc21] | 7-(2-((7-chloro-2-(2,2,2- trifluoroethyl)-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 126 | 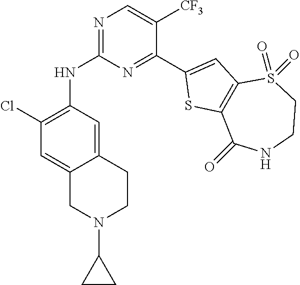 [SMILES: O=C1NCCS(=O)(=O)c2cc(-c3nc(Nc4cc5c(cc4Cl)CN(C4CC4)CC5)ncc3C(F)(F)F)sc21] | 7-(2-((7-chloro-2-cyclopropyl- 1,2,3,4-tetrahydroisoquinolin-6- yl)amino)-5- (trifluoromethyl)pyrimidin-4-yl)- 3,4-dihydrothieno[2,3- f][1,4]thiazepin-5(2H)-one 1,1- dioxide |
| 127 | 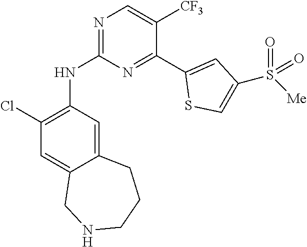 [SMILES: CS(=O)(=O)c1csc(-c2nc(Nc3cc4c(cc3Cl)CNCCC4)ncc2C(F)(F)F)c1] | 8-chloro-N-(4-(4- (methylsulfonyl)thiophen-2-yl)-5- (trifluoromethyl)pyrimidin-2-yl)- 2,3,4,5-tetrahydro-1H- benzo[c]azepin-7-amine |
| 128 | 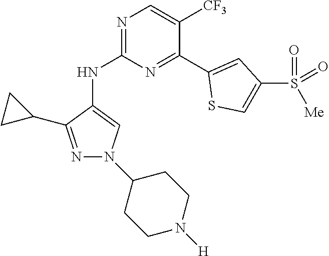 [SMILES: CS(=O)(=O)c1csc(-c2nc(Nc3cn(C4CCNCC4)nc3C3CC3)ncc2C(F)(F)F)c1] | N-(3-Cyclopropyl-1-(piperidin-4- yl)-1H-pyrazol-4-yl)-4-(4- (methylsulfonyl)thiophen-2-yl)-5- (trifluoromethyl)pyrimidin-2-amine |
| 129 | 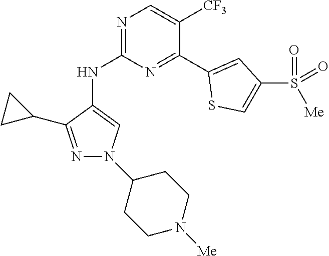 [SMILES: CN1CCC(n2cc(Nc3ncc(C(F)(F)F)c(-c4cc(S(C)(=O)=O)cs4)n3)c(C3CC3)n2)CC1] | N-(3-cyclopropyl-1-(1- methylpiperidin-4-yl)-1H-pyrazol- 4-yl)-4-(4- (methylsulfonyl)thiophen-2-yl)-5- (trifluoromethyl)pyrimidin-2-amine |
| 130 | 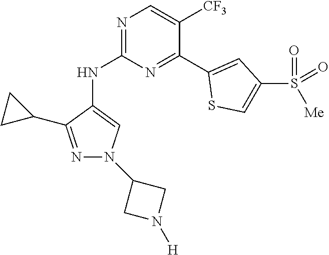 [SMILES: CS(=O)(=O)c1csc(-c2nc(Nc3cn(C4CNC4)nc3C3CC3)ncc2C(F)(F)F)c1] | N-(1-(azetidin-3-yl)-3-cyclopropyl- 1H-pyrazol-4-yl)-4-(4- (methylsulfonyl)thiophen-2-yl)-5- (trifluoromethyl)pyrimidin-2-amine |
| 131 | 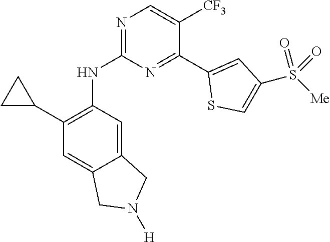 [SMILES: CS(=O)(=O)c1csc(-c2nc(Nc3cc4c(cc3C3CC3)CNC4)ncc2C(F)(F)F)c1] | 6-cyclopropyl-N-(4-(4- (methylsulfonyl)thiophen-2-yl)-5- (trifluoromethyl)pyrimidin-2- yl)isoindolin-5-amine |
| 132 | 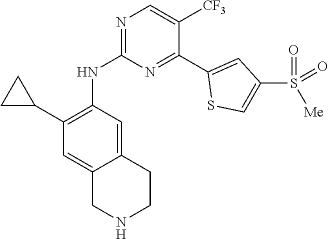 [SMILES: CS(=O)(=O)c1csc(-c2nc(Nc3cc4c(cc3C3CC3)CNCC4)ncc2C(F)(F)F)c1] | 7-cyclopropyl-N-(4-(4- (methylsulfonyl)thiophen-2-yl)-5- (trifluoromethyl)pyrimidin-2-yl)- 1,2,3,4-tetrahydroisoquinolin-6- amine |
| 133 | 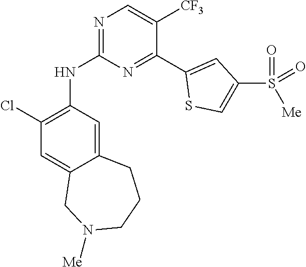 [SMILES: CN1CCCc2cc(Nc3ncc(C(F)(F)F)c(-c4cc(S(C)(=O)=O)cs4)n3)c(Cl)cc2C1] | 8-chloro-2-methyl-N-(4-(4- (methylsulfonyl)thiophen-2-yl)-5- (trifluoromethyl)pyrimidin-2-yl)- 2,3,4,5-tetrahydro-1H- benzo[c]azepin-7-amine |
| 134 | 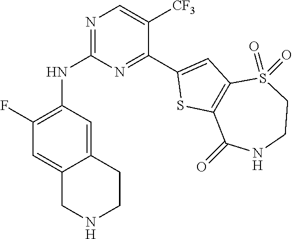 [SMILES: O=C1NCCS(=O)(=O)c2cc(-c3nc(Nc4cc5c(cc4F)CNCC5)ncc3C(F)(F)F)sc21] | 7-(2-((7-fluoro-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 135 | 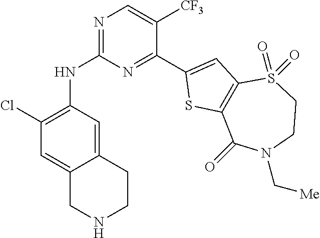 [SMILES: CCN1CCS(=O)(=O)c2cc(-c3nc(Nc4cc5c(cc4Cl)CNCC5)ncc3C(F)(F)F)sc2C1=O] | 7-(2-((7-chloro-1,2,3,4- tetrahydroisoquinolin-6-yl)amino)- 5-(trifluoromethyl)pyrimidin-4-yl)- 4-ethyl-3,4-dihydrothieno[2,3- f][1,4]thiazepin-5(2H)-one 1,1- dioxide |
| 136 | 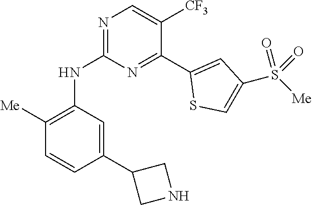 [SMILES: Cc1ccc(C2CNC2)cc1Nc1ncc(C(F)(F)F)c(-c2cc(S(C)(=O)=O)cs2)n1] | N-(5-(azetidin-3-yl)-2- methylphenyl)-4-(4- (methylsulfonyl)thiophen-2-yl)-5- (trifluoromethyl)pyrimidin-2-amine |
| 137 | 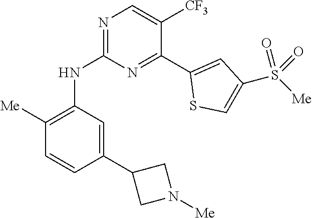 [SMILES: Cc1ccc(C2CN(C)C2)cc1Nc1ncc(C(F)(F)F)c(-c2cc(S(C)(=O)=O)cs2)n1] | N-(2-Methyl-5-(1-methylazetidin- 3-yl)phenyl)-4-(4- (methylsulfonyl)thiophen-2-yl)-5- (trifluoromethyl)pyrimidin-2-amine |
| 138 | 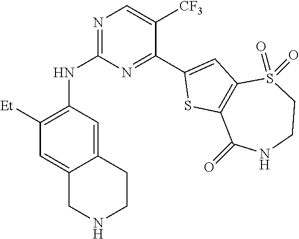 [SMILES: CCc1cc2c(cc1Nc1ncc(C(F)(F)F)c(-c3cc4c(s3)C(=O)NCCS4(=O)=O)n1)CCNC2] | 7-(2-((7-ethyl-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 139 | 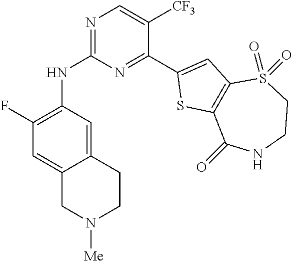 [SMILES: CN1CCc2cc(Nc3ncc(C(F)(F)F)c(-c4cc5c(s4)C(=O)NCCS5(=O)=O)n3)c(F)cc2C1] | 7-(2-((7-fluoro-2-methyl-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 140 | 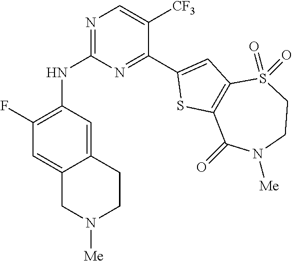 [SMILES: CN1CCc2cc(Nc3ncc(C(F)(F)F)c(-c4cc5c(s4)C(=O)N(C)CCS5(=O)=O)n3)c(F)cc2C1] | 7-(2-((7-fluoro-2-methyl-1,2,3,4- tetrahydroisoquinolin-6-yl)amino)- 5-(trifluoromethyl)pyrimidin-4-yl)- 4-methyl-3,4-dihydrothieno[2,3- f][1,4]thiazepin-5(2H)-one 1,1- dioxide |
| 141 | 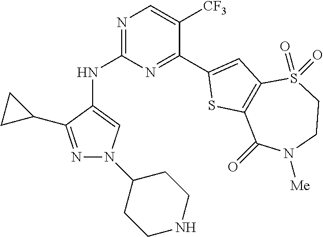 [SMILES: CN1CCS(=O)(=O)c2cc(-c3nc(Nc4cn(C5CCNCC5)nc4C4CC4)ncc3C(F)(F)F)sc2C1=O] | 7-(2-((3-cyclopropyl-1-(piperidin- 4-yl)-1H-pyrazol-4-yl)amino)-5- (trifluoromethyl)pyrimidin-4-y1)-4- methyl-3,4-dihydrothieno[2,3- f][1,4]thiazepin-5(2H)-one 1,1- dioxide |
| 142 | 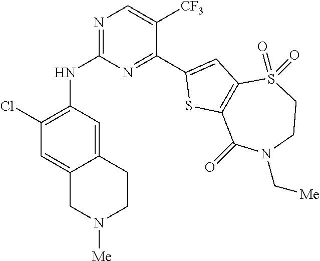 [SMILES: CCN1CCS(=O)(=O)c2cc(-c3nc(Nc4cc5c(cc4Cl)CN(C)CC5)ncc3C(F)(F)F)sc2C1=O] | 7-(2-((7-chloro-2-methyl-1,2,3,4- tetrahydroisoquinolin-6-yl)amino)- 5-(trifluoromethyl)pyrimidin-4-yl)- 4-ethyl-3,4-dihydrothieno[2,3- f][1,4]thiazepin-5(2H)-one 1,1- dioxide |
| 143 | 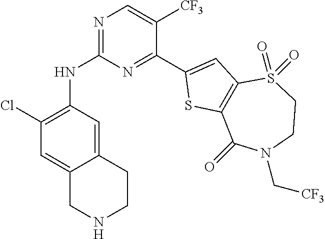 [SMILES: O=C1c2sc(-c3nc(Nc4cc5c(cc4Cl)CNCC5)ncc3C(F)(F)F)cc2S(=O)(=O)CCN1CC(F)(F)F] | 7-(2-((7-chloro-1,2,3,4- tetrahydroisoquinolin-6-yl)amino)- 5-(trifluoromethyl)pyrimidin-4-yl)- 4-(2,2,2-trifluoroethyl)-3,4- dihydrothieno[2,3-f][1,4]thiazepin- 5(2H)-one 1,1-dioxide |
| 144 | 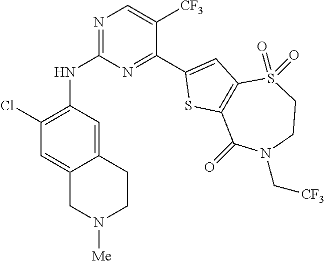 [SMILES: CN1CCc2cc(Nc3ncc(C(F)(F)F)c(-c4cc5c(s4)C(=O)N(CC(F)(F)F)CCS5(=O)=O)n3)c(Cl)cc2C1] | 7-(2-((7-chloro-2-methyl-1,2,3,4- tetrahydroisoquinolin-6-yl)amino)- 5-(trifluoromethyl)pyrimidin-4-yl)- 4-(2,2,2-trifluoroethyl)-3,4- dihydrothieno[2,3-f][1,4]thiazepin- 5(2H)-one 1,1-dioxide |
| 145 | 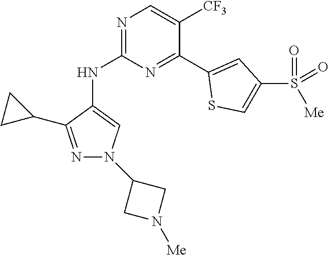 [SMILES: CN1CC(n2cc(Nc3ncc(C(F)(F)F)c(-c4cc(S(C)(=O)=O)cs4)n3)c(C3CC3)n2)C1] | N-(3-cyclopropyl-1-(1- methylazetidin-3-yl)-1H-pyrazol-4- yl)-4-(4-(methylsulfonyl)thiophen- 2-yl)-5-(trifluoromethyl)pyrimidin- 2-amine |
| 146 | 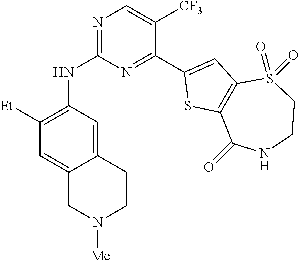 [SMILES: CCc1cc2c(cc1Nc1ncc(C(F)(F)F)c(-c3cc4c(s3)C(=O)NCCS4(=O)=O)n1)CCN(C)C2] | 7-(2-((7-ethyl-2-methyl-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 147 | 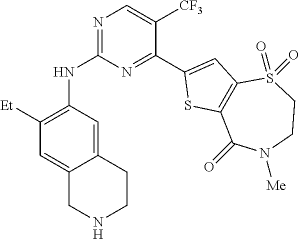 [SMILES: CCc1cc2c(cc1Nc1ncc(C(F)(F)F)c(-c3cc4c(s3)C(=O)N(C)CCS4(=O)=O)n1)CCNC2] | 7-(2-((7-ethyl-1,2,3,4- tetrahydroisoquinolin-6-yl)amino)- 5-(trifluoromethyl)pyrimidin-4-yl)- 4-methyl-3,4-dihydrothieno[2,3- f][1,4]thiazepin-5(2H)-one 1,1- dioxide |
| 148 | 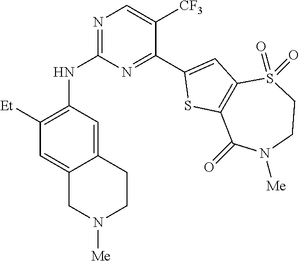 [SMILES: CCc1cc2c(cc1Nc1ncc(C(F)(F)F)c(-c3cc4c(s3)C(=O)N(C)CCS4(=O)=O)n1)CCN(C)C2] | 7-(2-((7-ethyl-2-methyl-1,2,3,4- tetrahydroisoquinolin-6-yl)amino)- 5-(trifluoromethyl)pyrimidin-4-yl)- 4-methyl-3,4-dihydrothieno[2,3- f][1,4]thiazepin-5(2H)-one 1,1- dioxide |
| 149 | 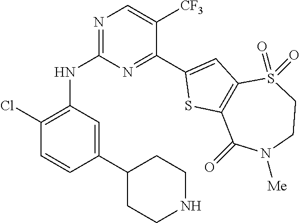 [SMILES: CN1CCS(=O)(=O)c2cc(-c3nc(Nc4cc(C5CCNCC5)ccc4Cl)ncc3C(F)(F)F)sc2C1=O] | 7-(2-((2-chloro-5-(piperidin-4- yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 150 | 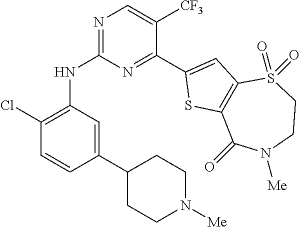 [SMILES: CN1CCC(c2ccc(Cl)c(Nc3ncc(C(F)(F)F)c(-c4cc5c(s4)C(=O)N(C)CCS5(=O)=O)n3)c2)CC1] | 7-(2-((2-chloro-5-(1- methylpiperidin-4- yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 151 | 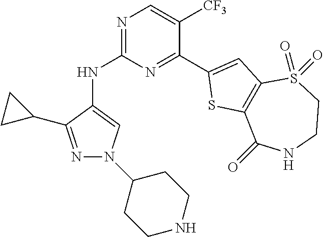 [SMILES: O=C1NCCS(=O)(=O)c2cc(-c3nc(Nc4cn(C5CCNCC5)nc4C4CC4)ncc3C(F)(F)F)sc21] | 7-(2-((3-cyclopropyl-1-(piperidin- 4-yl)-1H-pyrazol-4-yl)amino)-5- (trifluoromethyl)pyrimidin-4-yl)- 3,4-dihydrothieno[2,3- f][1,4]thiazepin-5(2H)-one 1,1- dioxide |
| 152 | 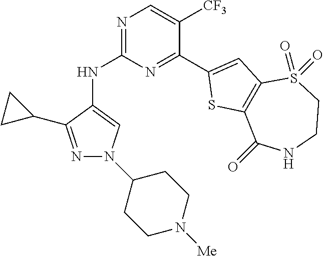 [SMILES: CN1CCC(n2cc(Nc3ncc(C(F)(F)F)c(-c4cc5c(s4)C(=O)NCCS5(=O)=O)n3)c(C3CC3)n2)CC1] | 7-(2-((3-cyclopropyl-1-(1- methylpiperidin-4-yl)-1H-pyrazol- 4-yl)amino)-5- (trifluoromethyl)pyrimidin-4-yl)- 3,4-dihydrothieno[2,3- f][1,4]thiazepin-5(2H)-one 1,1- dioxide |
| 153 | 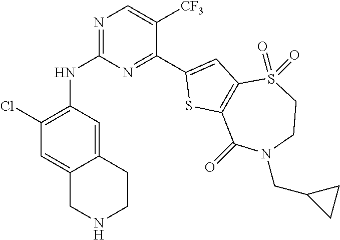 [SMILES: O=C1c2sc(-c3nc(Nc4cc5c(cc4Cl)CNCC5)ncc3C(F)(F)F)cc2S(=O)(=O)CCN1CC1CC1] | 7-(2-((7-chloro-1,2,3,4- tetrahydroisoquinolin-6-yl)amino)- 5-(trifluoromethyl)pyrimidin-4-yl)- 4-(cyclopropylmethyl)-3,4- dihydrothieno[2,3-f][1,4]thiazepin- 5(2H)-one 1,1-dioxide |
| 154 | 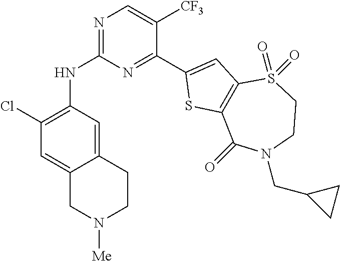 [SMILES: CN1CCc2cc(Nc3ncc(C(F)(F)F)c(-c4cc5c(s4)C(=O)N(CC4CC4)CCS5(=O)=O)n3)c(Cl)cc2C1] | 7-(2-((7-chloro-2-methyl-1,2,3,4- tetrahydroisoquinolin-6-yl)amino)- 5-(trifluoromethyl)pyrimidin-4-yl)- 4-(cyclopropylmethyl)-3,4- dihydrothieno[2,3-f][1,4]thiazepin- 5(2H)-one 1,1-dioxide |
| 155 | 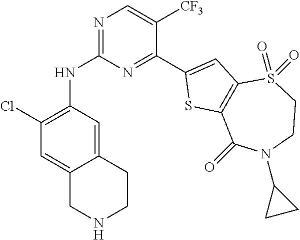 [SMILES: O=C1c2sc(-c3nc(Nc4cc5c(cc4Cl)CNCC5)ncc3C(F)(F)F)cc2S(=O)(=O)CCN1C1CC1] | 7-(2-((7-chloro-1,2,3,4- tetrahydroisoquinolin-6-yl)amino)- 5-(trifluoromethyl)pyrimidin-4-yl)- 4-cyclopropyl-3,4- dihydrothieno[2,3-f][1,4]thiazepin- 5(2H)-one 1,1-dioxide |
| 156 | 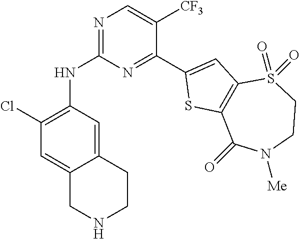 [SMILES: CN1CCS(=O)(=O)c2cc(-c3nc(Nc4cc5c(cc4Cl)CNCC5)ncc3C(F)(F)F)sc2C1=O] | 7-(2-((7-chloro-1,2,3,4- tetrahydroisoquinolin-6-yl)amino)- 5-(trifluoromethyl)pyrimidin-4-yl)- 4-methyl-3,4-dihydrothieno[2,3- f][1,4]thiazepin-5(2H)-one 1,1- dioxide |
| 157 | 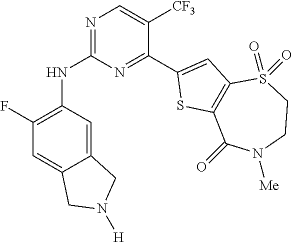 [SMILES: CN1CCS(=O)(=O)c2cc(-c3nc(Nc4cc5c(cc4F)CNC5)ncc3C(F)(F)F)sc2C1=O] | 7-(2-((6-fluoroisoindolin-5- yl)amino)-5- (trifluoromethyl)pyrimidin-4-yl)-4- methyl-3,4-dihydrothieno[2,3- f][1,4]thiazepin-5(2H)-one 1,1- dioxide |
| 158 | 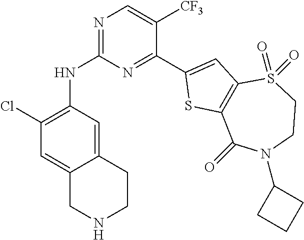 [SMILES: O=C1c2sc(-c3nc(Nc4cc5c(cc4Cl)CNCC5)ncc3C(F)(F)F)cc2S(=O)(=O)CCN1C1CCC1] | 7-(2-((7-chloro-1,2,3,4- tetrahydroisoquinolin-6-yl)amino)- 5-(trifluoromethyl)pyrimidin-4-yl)- 4-cyclobutyl-3,4- dihydrothieno[2,3-f][1,4]thiazepin- 5(2H)-one 1,1-dioxide |
| 159 | 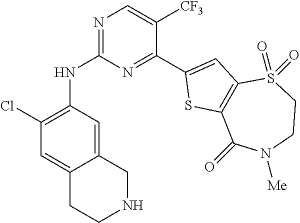 [SMILES: CN1CCS(=O)(=O)c2cc(-c3nc(Nc4cc5c(cc4Cl)CCNC5)ncc3C(F)(F)F)sc2C1=O] | 7-(2-((6-chloro-1,2,3,4- tetrahydroisoquinolin-7-yl)amino)- 5-(trifluoromethyl)pyrimidin-4-yl)- 4-methyl-3,4-dihydrothieno[2,3- f][1,4]thiazepin-5(2H)-one 1,1- dioxide |
| 160 | 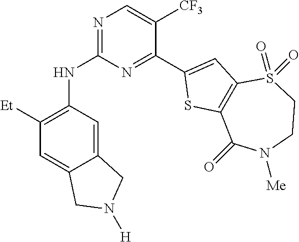 [SMILES: CCc1cc2c(cc1Nc1ncc(C(F)(F)F)c(-c3cc4c(s3)C(=O)N(C)CCS4(=O)=O)n1)CNC2] | 7-(2-((6-ethylisoindolin-5- yl)amino)-5- (trifluoromethyl)pyrimidin-4-yl)-4- methyl-3,4-dihydrothieno[2,3- f][1,4]thiazepin-5(2H)-one 1,1- dioxide |
| 161 | 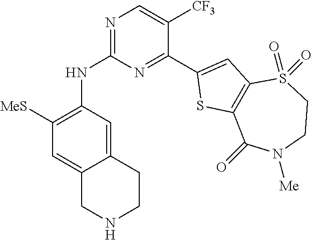 [SMILES: CSc1cc2c(cc1Nc1ncc(C(F)(F)F)c(-c3cc4c(s3)C(=O)N(C)CCS4(=O)=O)n1)CCNC2] | 4-methyl-7-(2-((7-(methylthio)- 1,2,3,4-tetrahydroisoquinolin-6- yl)amino)-5- (trifluoromethyl)pyrimidin-4-yl)- 3,4-dihydrothieno[2,3- f][1,4]thiazepin-5(2H)-one 1,1- dioxide |
| 162 | 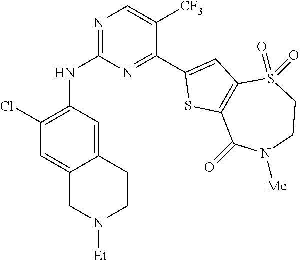 [SMILES: CCN1CCc2cc(Nc3ncc(C(F)(F)F)c(-c4cc5c(s4)C(=O)N(C)CCS5(=O)=O)n3)c(Cl)cc2C1] | 7-(2-((7-chloro-2-ethyl-1,2,3,4- tetrahydroisoquinolin-6-yl)amino)- 5-(trifluoromethyl)pyrimidin-4-yl)- 4-methyl-3,4-dihydrothieno[2,3- f][1,4]thiazepin-5(2H)-one 1,1- dioxide |
| 163 | 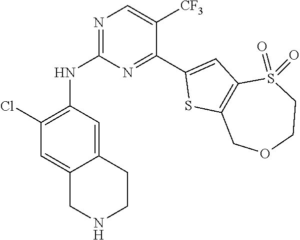 [SMILES: O=S1(=O)CCOCc2sc(-c3nc(Nc4cc5c(cc4Cl)CNCC5)ncc3C(F)(F)F)cc21] | 7-(2-((7-chloro-1,2,3,4- tetrahydroisoquinolin-6-yl)amino)- 5-(trifluoromethyl)pyrimidin-4-yl)- 2,3-dihydro-5H-thieno[3,2- e][1,4]oxathiepine 1,1-dioxide |
| 164 | 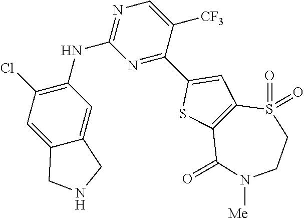 [SMILES: CN1CCS(=O)(=O)c2cc(-c3nc(Nc4cc5c(cc4Cl)CNC5)ncc3C(F)(F)F)sc2C1=O] | 7-(2-((6-chloroisoindolin-5- yl)amino)-5- (trifluoromethyl)pyrimidin-4-yl)-4- methyl-3,4-dihydrothieno[2,3- f][1,4]thiazepin-5(2H)-one 1,1- dioxide |
| 165 | 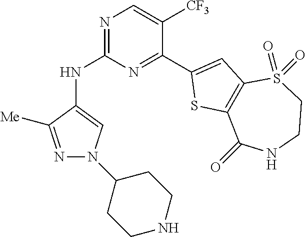 [SMILES: Cc1nn(C2CCNCC2)cc1Nc1ncc(C(F)(F)F)c(-c2cc3c(s2)C(=O)NCCS3(=O)=O)n1] | 7-(2-((3-methyl-1-(piperidin-4-yl)- 1H-pyrazol-4-yl)amino)-5- (trifluoromethyl)pyrimidin-4-yl)- 3,4-dihydrothieno[2,3- f][1,4]thiazepin-5(2H)-one 1,1- dioxide |
| 166 | 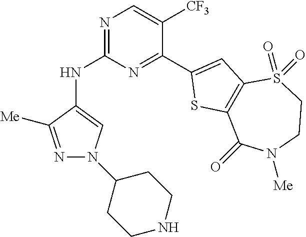 [SMILES: Cc1nn(C2CCNCC2)cc1Nc1ncc(C(F)(F)F)c(-c2cc3c(s2)C(=O)N(C)CCS3(=O)=O)n1] | 4-methyl-7-(2-((3-methyl-1- (piperidin-4-yl)-1H-pyrazol-4- yl)amino)-5- (trifluoromethyl)pyrimidin-4-yl)- 3,4-dihydrothieno[2,3- f][1,4]thiazepin-5(2H)-one 1,1- dioxide |
| 167 | 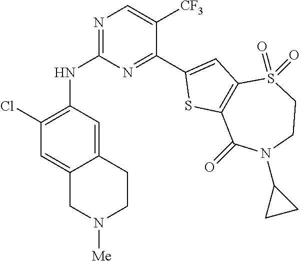 [SMILES: CN1CCc2cc(Nc3ncc(C(F)(F)F)c(-c4cc5c(s4)C(=O)N(C4CC4)CCS5(=O)=O)n3)c(Cl)cc2C1] | 7-(2-((7-chloro-2-methyl-1,2,3,4- tetrahydroisoquinolin-6-yl)amino)- 5-(trifluoromethyl)pyrimidin-4-yl)- 4-cyclopropyl-3,4- dihydrothieno[2,3-f][1,4]thiazepin- 5(2H)-one 1,1-dioxide |
| 168 | 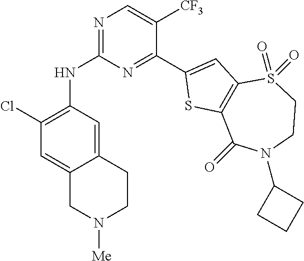 [SMILES: CN1CCc2cc(Nc3ncc(C(F)(F)F)c(-c4cc5c(s4)C(=O)N(C4CCC4)CCS5(=O)=O)n3)c(Cl)cc2C1] | 7-(2-((7-chloro-2-methyl-1,2,3,4- tetrahydroisoquinolin-6-yl)amino)- 5-(trifluoromethyl)pyrimidin-4-yl)- 4-cyclobutyl-3,4- dihydrothieno[2,3-f][1,4]thiazepin- 5(2H)-one 1,1-dioxide |
| 169 | 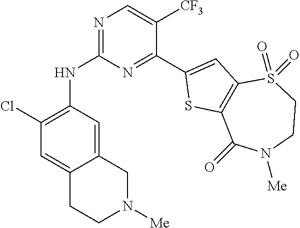 [SMILES: CN1CCc2cc(Cl)c(Nc3ncc(C(F)(F)F)c(-c4cc5c(s4)C(=O)N(C)CCS5(=O)=O)n3)cc2C1] | 7-(2-((7-chloro-2-methyl-1,2,3,4- tetrahydroisoquinolin-6-yl)amino)- 5-(trifluoromethyl)pyrimidin-4-yl)- 4-cyclobutyl-3,4- dihydrothieno[2,3-f][1,4]thiazepin- 5(2H)-one 1,1-dioxide |
| 170 | 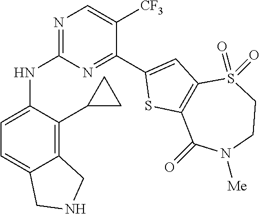 [SMILES: CN1CCS(=O)(=O)c2cc(-c3nc(Nc4ccc5c(c4C4CC4)CNC5)ncc3C(F)(F)F)sc2C1=O] | 7-(2-((4-cyclopropylisoindolin-5- yl)amino)-5- (trifluoromethyl)pyrimidin-4-yl)-4- methyl-3,4-dihydrothieno[2,3- f][1,4]thiazepin-5(2H)-one 1,1- dioxide |
| 171 | 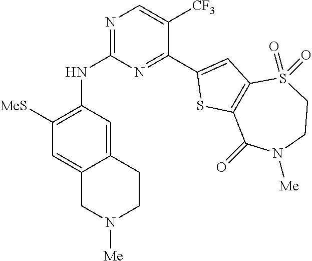 [SMILES: CSc1cc2c(cc1Nc1ncc(C(F)(F)F)c(-c3cc4c(s3)C(=O)N(C)CCS4(=O)=O)n1)CCN(C)C2] | 4-methyl-7-(2-((2-methyl-7- (methylthio)-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 172 | 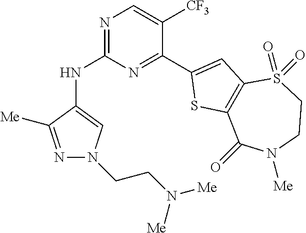 [SMILES: Cc1nn(CCN(C)C)cc1Nc1ncc(C(F)(F)F)c(-c2cc3c(s2)C(=O)N(C)CCS3(=O)=O)n1] | 7-(2-((1-(2-(dimethylamino)ethyl)- 3-methyl-1H-pyrazol-4-yl)amino)- 5-(trifluoromethyl)pyrimidin-4-yl)- 4-methyl-3,4-dihydrothieno[2,3- f][1,4]thiazepin-5(2H)-one 1,1- dioxide |
| 173 | 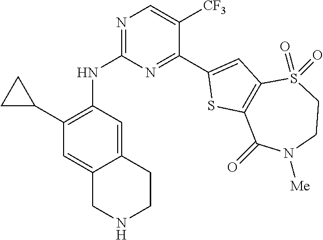 [SMILES: CN1CCS(=O)(=O)c2cc(-c3nc(Nc4cc5c(cc4C4CC4)CNCC5)ncc3C(F)(F)F)sc2C1=O] | 7-(2-((7-cyclopropyl-1,2,3,4- tetrahydroisoquinolin-6-yl)amino)- 5-(trifluoromethyl)pyrimidin-4-yl)- 4-methyl-3,4-dihydrothieno[2,3- f][1,4]thiazepin-5(2H)-one 1,1- dioxide |
| 174 | 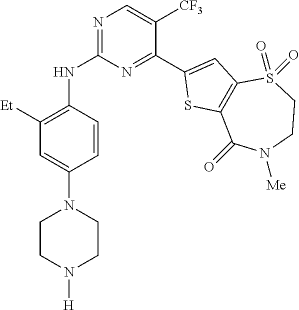 [SMILES: CCc1cc(N2CCNCC2)ccc1Nc1ncc(C(F)(F)F)c(-c2cc3c(s2)C(=O)N(C)CCS3(=O)=O)n1] | 7-(2-((2-ethyl-4-(piperazin-1- yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 175 | 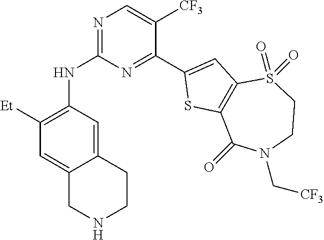 [SMILES: CCc1cc2c(cc1Nc1ncc(C(F)(F)F)c(-c3cc4c(s3)C(=O)N(CC(F)(F)F)CCS4(=O)=O)n1)CCNC2] | 7-(2-((7-ethyl-1,2,3,4- tetrahydroisoquinolin-6-yl)amino)- 5-(trifluoromethyl)pyrimidin-4-yl)- 4-(2,2,2-trifluoroethyl)-3,4- dihydrothieno[2,3-f][1,4]thiazepin- 5(2H)-one 1,1-dioxide |
| 176 | 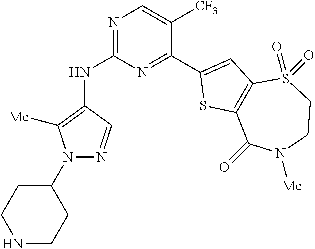 [SMILES: Cc1c(Nc2ncc(C(F)(F)F)c(-c3cc4c(s3)C(=O)N(C)CCS4(=O)=O)n2)cnn1C1CCNCC1] | 4-methyl-7-(2-((5-methyl-1- (piperidin-4-yl)-1H-pyrazol-4- yl)amino)-5- (trifluoromethyl)pyrimidin-4-yl)- 3,4-dihydrothieno[2,3- f][1,4]thiazepin-5(2H)-one 1,1- dioxide |
| 177 | 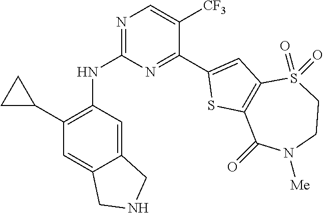 [SMILES: CN1CCS(=O)(=O)c2cc(-c3nc(Nc4cc5c(cc4C4CC4)CNC5)ncc3C(F)(F)F)sc2C1=O] | 7-(2-((6-cyclopropylisoindolin-5- yl)amino)-5- (trifluoromethyl)pyrimidin-4-yl)-4- methyl-3,4-dihydrothieno[2,3- f][1,4]thiazepin-5(2H)-one 1,1- dioxide |
| 178 | 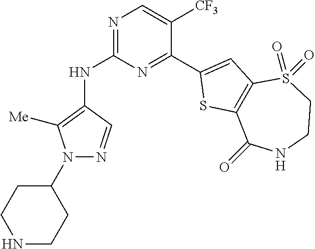 [SMILES: Cc1c(Nc2ncc(C(F)(F)F)c(-c3cc4c(s3)C(=O)NCCS4(=O)=O)n2)cnn1C1CCNCC1] | 7-(2-((5-methyl-1-(piperidin-4-yl)- 1H-pyrazol-4-yl)amino)-5- (trifluoromethyl)pyrimidin-4-yl)- 3,4-dihydrothieno[2,3- f][1,4]thiazepin-5(2H)-one 1,1- dioxide |
| 179 | 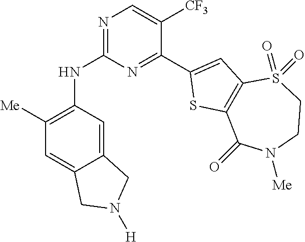 [SMILES: Cc1cc2c(cc1Nc1ncc(C(F)(F)F)c(-c3cc4c(s3)C(=O)N(C)CCS4(=O)=O)n1)CNC2] | 7-(2-((2-ethyl-6-methylisoindolin- 5-yl)amino)-5- (trifluoromethyl)pyrimidin-4-yl)-4- methyl-3,4-dihydrothieno[2,3- f][1,4]thiazepin-5(2H)-one 1,1- dioxide |
| 180 | 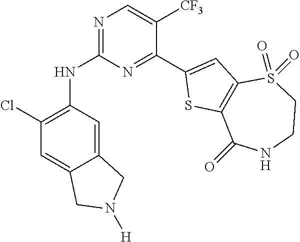 [SMILES: O=C1NCCS(=O)(=O)c2cc(-c3nc(Nc4cc5c(cc4Cl)CNC5)ncc3C(F)(F)F)sc21] | 7-(2-((6-chloroisoindolin-5- yl)amino)-5- (trifluoromethyl)pyrimidin-4-yl)- 3,4-dihydrothieno[2,3- f][1,4]thiazepin-5(2H)-one 1,1- dioxide |
| 181 | 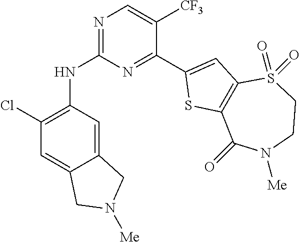 [SMILES: CN1Cc2cc(Cl)c(Nc3ncc(C(F)(F)F)c(-c4cc5c(s4)C(=O)N(C)CCS5(=O)=O)n3)cc2C1] | 7-(2-((6-chloro-2-methylisoindolin- 5-yl)amino)-5- (trifluoromethyl)pyrimidin-4-yl)-4- methyl-3,4-dihydrothieno[2,3- f][1,4]thiazepin-5(2H)-one 1,1- dioxide |
| 182 | 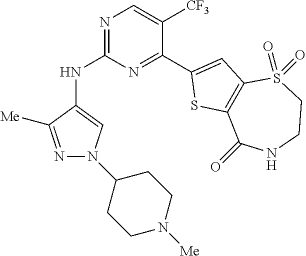 [SMILES: Cc1nn(C2CCN(C)CC2)cc1Nc1ncc(C(F)(F)F)c(-c2cc3c(s2)C(=O)NCCS3(=O)=O)n1] | 7-(2-((3-methyl-1-(1- methylpiperidin-4-yl)-1H-pyrazol- 4-yl)amino)-5- (trifluoromethyl)pyrimidin-4-yl)- 3,4-dihydrothieno[2,3- f][1,4]thiazepin-5(2H)-one 1,1- dioxide |
| 183 | 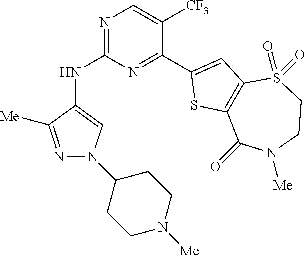 [SMILES: Cc1nn(C2CCN(C)CC2)cc1Nc1ncc(C(F)(F)F)c(-c2cc3c(s2)C(=O)N(C)CCS3(=O)=O)n1] | 4-methyl-7-(2-((3-methyl-1-(1- methylpiperidin-4-yl)-1H-pyrazol- 4-yl)amino)-5- (trifluoromethyl)pyrimidin-4-yl)- 3,4-dihydrothieno[2,3- f][1,4]thiazepin-5(2H)-one 1,1- dioxide |
| 184 | 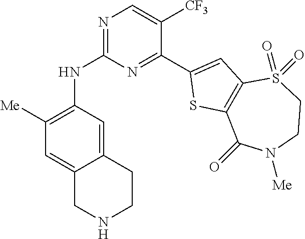 [SMILES: Cc1cc2c(cc1Nc1ncc(C(F)(F)F)c(-c3cc4c(s3)C(=O)N(C)CCS4(=O)=O)n1)CCNC2] | 4-methyl-7-(2-((7-methyl-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 185 | 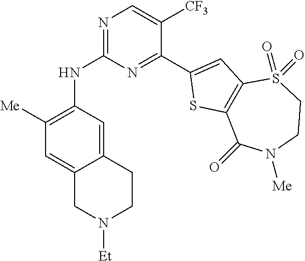 [SMILES: CCN1CCc2cc(Nc3ncc(C(F)(F)F)c(-c4cc5c(s4)C(=O)N(C)CCS5(=O)=O)n3)c(C)cc2C1] | 7-(2-((2-ethyl-7-methyl-1,2,3,4- tetrahydroisoquinolin-6-yl)amino)- 5-(trifluoromethyl)pyrimidin-4-yl)- 4-methyl-3,4-dihydrothieno[2,3- f][1,4]thiazepin-5(2H)-one 1,1- dioxide |
| 186 | 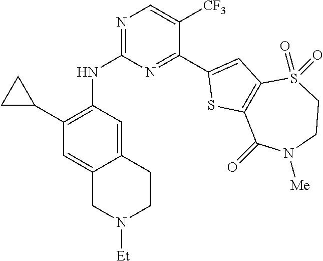 [SMILES: CCN1CCc2cc(Nc3ncc(C(F)(F)F)c(-c4cc5c(s4)C(=O)N(C)CCS5(=O)=O)n3)c(C3CC3)cc2C1] | 7-(2-((7-cyclopropyl-2-ethyl- 1,2,3,4-tetrahydroisoquinolin-6- yl)amino)-5- (trifluoromethyl)pyrimidin-4-yl)-4- methyl-3,4-dihydrothieno[2,3- f][1,4]thiazepin-5(2H)-one 1,1- dioxide |
| 187 | 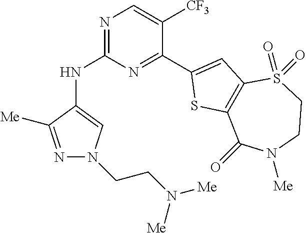 [SMILES: Cc1nn(CCN(C)C)cc1Nc1ncc(C(F)(F)F)c(-c2cc3c(s2)C(=O)N(C)CCS3(=O)=O)n1] | 7-(2-((1-(2-(dimethylamino)ethyl)- 3-methyl-1H-pyrazol-4-yl)amino)- 5-(trifluoromethyl)pyrimidin-4-yl)- 4-methyl-3,4-dihydrothieno[2,3- f][1,4]thiazepin-5(2H)-one 1,1- dioxide |
| 188 | 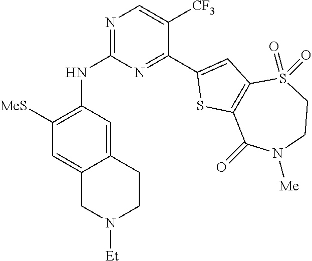 [SMILES: CCN1CCc2cc(Nc3ncc(C(F)(F)F)c(-c4cc5c(s4)C(=O)N(C)CCS5(=O)=O)n3)c(SC)cc2C1] | 7-(2-((2-ethyl-7-(methylthio)- 1,2,3,4-tetrahydroisoquinolin-6- yl)amino)-5- (trifluoromethyl)pyrimidin-4-yl)-4- methyl-3,4-dihydrothieno[2,3- f][1,4]thiazepin-5(2H)-one 1,1- dioxide |
| 189 | 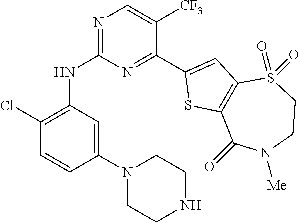 [SMILES: CN1CCS(=O)(=O)c2cc(-c3nc(Nc4cc(N5CCNCC5)ccc4Cl)ncc3C(F)(F)F)sc2C1=O] | 7-(2-((2-chloro-5-(piperazin-1- yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 190 | 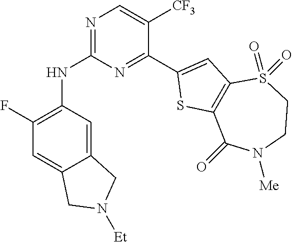 [SMILES: CCN1Cc2cc(F)c(Nc3ncc(C(F)(F)F)c(-c4cc5c(s4)C(=O)N(C)CCS5(=O)=O)n3)cc2C1] | 7-(2-((2-ethyl-6-fluoroisoindolin-5- yl)amino)-5- (trifluoromethyl)pyrimidin-4-yl)-4- methyl-3,4-dihydrothieno[2,3- f][1,4]thiazepin-5(2H)-one 1,1- dioxide |
| 191 | 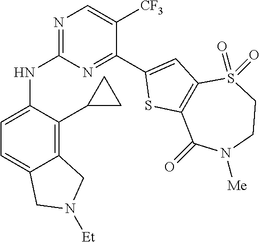 [SMILES: CCN1Cc2ccc(Nc3ncc(C(F)(F)F)c(-c4cc5c(s4)C(=O)N(C)CCS5(=O)=O)n3)c(C3CC3)c2C1] | 7-(2-((4-cyclopropyl-2- ethylisoindolin-5-yl)amino)-5- (trifluoromethyl)pyrimidin-4-yl)-4- methyl-3,4-dihydrothieno[2,3- f][1,4]thiazepin-5(2H)-one 1,1- dioxide |
| 192 | 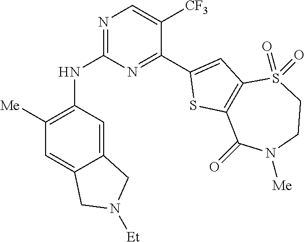 [SMILES: CCN1Cc2cc(C)c(Nc3ncc(C(F)(F)F)c(-c4cc5c(s4)C(=O)N(C)CCS5(=O)=O)n3)cc2C1] | 7-(2-((2-ethyl-6-methylisoindolin- 5-yl)amino)-5- (trifluoromethyl)pyrimidin-4-yl)-4- methyl-3,4-dihydrothieno[2,3- f][1,4]thiazepin-5(2H)-one 1,1- dioxide |
| 193 | 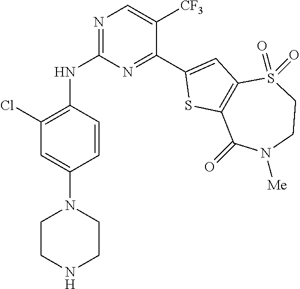 [SMILES: CN1CCS(=O)(=O)c2cc(-c3nc(Nc4ccc(N5CCNCC5)cc4Cl)ncc3C(F)(F)F)sc2C1=O] | 7-(2-((2-chloro-4-(piperazin-1- yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 194 | 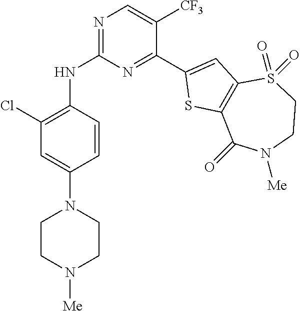 [SMILES: CN1CCN(c2ccc(Nc3ncc(C(F)(F)F)c(-c4cc5c(s4)C(=O)N(C)CCS5(=O)=O)n3)c(Cl)c2)CC1] | 7-(2-((2-chloro-4-(4- methylpiperazin-1- yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 195 | 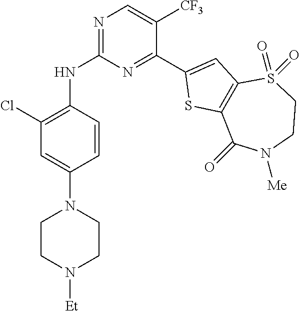 [SMILES: CCN1CCN(c2ccc(Nc3ncc(C(F)(F)F)c(-c4cc5c(s4)C(=O)N(C)CCS5(=O)=O)n3)c(Cl)c2)CC1] | 7-(2-((2-chloro-4-(4-ethylpiperazin- 1-yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 196 | 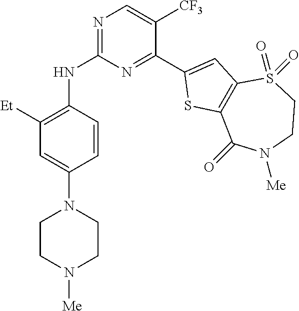 [SMILES: CCc1cc(N2CCN(C)CC2)ccc1Nc1ncc(C(F)(F)F)c(-c2cc3c(s2)C(=O)N(C)CCS3(=O)=O)n1] | 7-(2-((2-ethyl-4-(4- methylpiperazin-1- yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 197 | 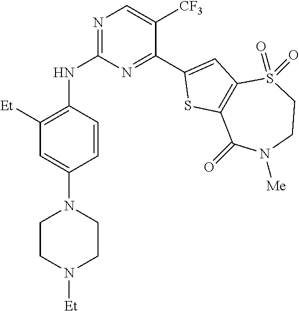 [SMILES: CCc1cc(N2CCN(CC)CC2)ccc1Nc1ncc(C(F)(F)F)c(-c2cc3c(s2)C(=O)N(C)CCS3(=O)=O)n1] | 7-(2-((2-ethyl-4-(4-ethylpiperazin- 1-yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 198 | 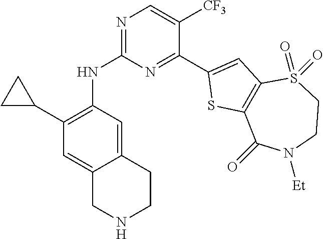 [SMILES: CCN1CCS(=O)(=O)c2cc(-c3nc(Nc4cc5c(cc4C4CC4)CNCC5)ncc3C(F)(F)F)sc2C1=O] | 7-(2-((7-cyclopropyl-1,2,3,4- tetrahydroisoquinolin-6-yl)amino)- 5-(trifluoromethyl)pyrimidin-4-yl)- 4-ethyl-3,4-dihydrothieno[2,3- f][1,4]thiazepin-5(2H)-one 1,1- dioxide |
| 199 | 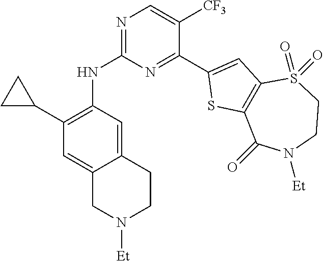 [SMILES: CCN1CCc2cc(Nc3ncc(C(F)(F)F)c(-c4cc5c(s4)C(=O)N(CC)CCS5(=O)=O)n3)c(C3CC3)cc2C1] | 7-(2-((7-cyclopropyl-2-ethyl- 1,2,3,4-tetrahydroisoquinolin-6- yl)amino)-5- (trifluoromethyl)pyrimidin-4-yl)-4- ethyl-3,4-dihydrothieno[2,3- f][1,4]thiazepin-5(2H)-one 1,1- dioxide |
| 200 | 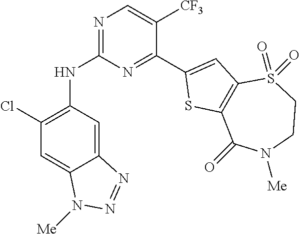 [SMILES: CN1CCS(=O)(=O)c2cc(-c3nc(Nc4cc5nnn(C)c5cc4Cl)ncc3C(F)(F)F)sc2C1=O] | 7-(2-((6-chloro-1-methyl-1H- benzo[d][1,2,3]triazol-5-yl)amino)- 5-(trifluoromethyl)pyrimidin-4-yl)- 4-methyl-3,4-dihydrothieno[2,3- f][1,4]thiazepin-5(2H)-one 1,1- dioxide |
| 201 | 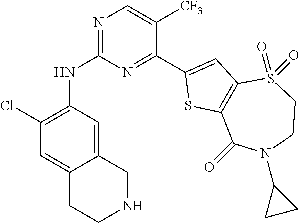 [SMILES: O=C1c2sc(-c3nc(Nc4cc5c(cc4Cl)CCNC5)ncc3C(F)(F)F)cc2S(=O)(=O)CCN1C1CC1] | 7-(2-((6-chloro-1,2,3,4- tetrahydroisoquinolin-7-yl)amino)- 5-(trifluoromethyl)pyrimidin-4-yl)- 4-cyclopropyl-3,4- dihydrothieno[2,3-f][1,4]thiazepin- 5(2H)-one 1,1-dioxide |
| 202 | 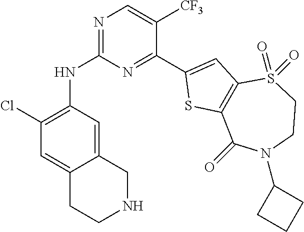 [SMILES: O=C1c2sc(-c3nc(Nc4cc5c(cc4Cl)CCNC5)ncc3C(F)(F)F)cc2S(=O)(=O)CCN1C1CCC1] | 7-(2-((6-chloro-1,2,3,4- tetrahydroisoquinolin-7-yl)amino)- 5-(trifluoromethyl)pyrimidin-4-yl)- 4-cyclopropyl-3,4- dihydrothieno[2,3-f][1,4]thiazepin- 5(2H)-one 1,1-dioxide |
| 203 | 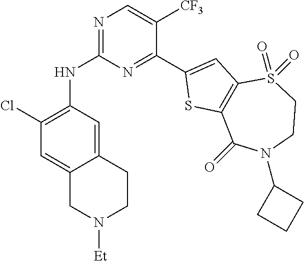 [SMILES: CCN1CCc2cc(Nc3ncc(C(F)(F)F)c(-c4cc5c(s4)C(=O)N(C4CCC4)CCS5(=O)=O)n3)c(Cl)cc2C1] | 7-(2-((7-chloro-2-ethyl-1,2,3,4- tetrahydroisoquinolin-6-yl)amino)- 5-(trifluoromethyl)pyrimidin-4-yl)- 4-cyclobutyl-3,4- dihydrothieno[2,3-f][1,4]thiazepin- 5(2H)-one 1,1-dioxide |
| 204 | 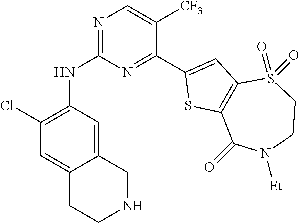 [SMILES: CCN1CCS(=O)(=O)c2cc(-c3nc(Nc4cc5c(cc4Cl)CCNC5)ncc3C(F)(F)F)sc2C1=O] | 7-(2-((6-chloro-1,2,3,4- tetrahydroisoquinolin-7-yl)amino)- 5-(trifluoromethyl)pyrimidin-4-yl)- 4-ethyl-3,4-dihydrothieno[2,3- f][1,4]thiazepin-5(2H)-one 1,1- dioxide |
| 205 | 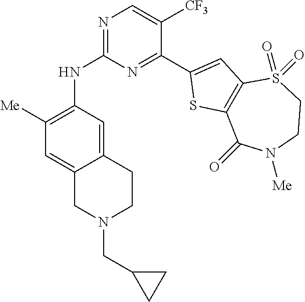 [SMILES: Cc1cc2c(cc1Nc1ncc(C(F)(F)F)c(-c3cc4c(s3)C(=O)N(C)CCS4(=O)=O)n1)CCN(CC1CC1)C2] | 7-(2-((2-(cyclopropylmethyl)-7- methyl-1,2,3,4- tetrahydroisoquinolin-6-yl)amino)- 5-(trifluoromethyl)pyrimidin-4-yl)- 4-methyl-3,4-dihydrothieno[2,3- f][1,4]thiazepin-5(2H)-one 1,1- dioxide |
| 206 | 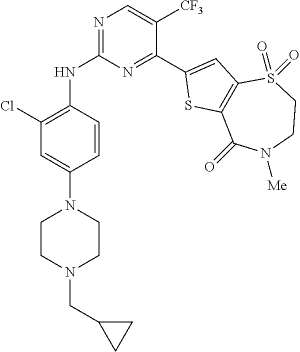 [SMILES: CN1CCS(=O)(=O)c2cc(-c3nc(Nc4ccc(N5CCN(CC6CC6)CC5)cc4Cl)ncc3C(F)(F)F)sc2C1=O] | 7-(2-((2-chloro-4-(4- (cyclopropylmethyl)piperazin-1- yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 207 | 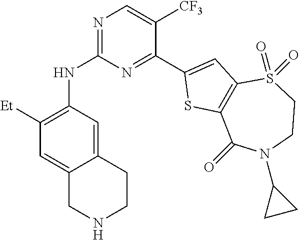 [SMILES: CCc1cc2c(cc1Nc1ncc(C(F)(F)F)c(-c3cc4c(s3)C(=O)N(C3CC3)CCS4(=O)=O)n1)CCNC2] | 4-cyclopropyl-7-(2-((7-ethyl- 1,2,3,4-tetrahydroisoquinolin-6- yl)amino)-5- (trifluoromethyl)pyrimidin-4-yl)- 3,4-dihydrothieno[2,3- f][1,4]thiazepin-5(2H)-one 1,1- dioxide |
| 208 | 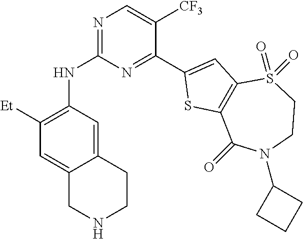 [SMILES: CCc1cc2c(cc1Nc1ncc(C(F)(F)F)c(-c3cc4c(s3)C(=O)N(C3CCC3)CCS4(=O)=O)n1)CCNC2] | 4-cyclobutyl-7-(2-((7-ethyl-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 209 | 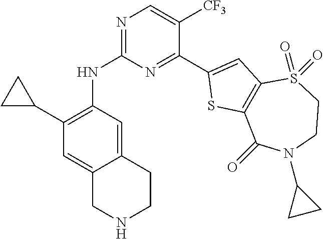 [SMILES: O=C1c2sc(-c3nc(Nc4cc5c(cc4C4CC4)CNCC5)ncc3C(F)(F)F)cc2S(=O)(=O)CCN1C1CC1] | 4-cyclopropyl-7-(2-((7- cyclopropyl-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 210 | 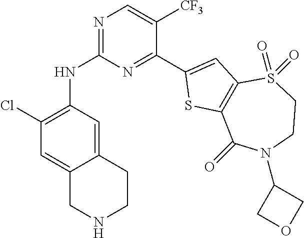 [SMILES: O=C1c2sc(-c3nc(Nc4cc5c(cc4Cl)CNCC5)ncc3C(F)(F)F)cc2S(=O)(=O)CCN1C1COC1] | 7-(2-((7-chloro-1,2,3,4- tetrahydroisoquinolin-6-yl)amino)- 5-(trifluoromethyl)pyrimidin-4-yl)- 4-(oxetan-3-yl)-3,4- dihydrothieno[2,3-f][1,4]thiazepin- 5(2H)-one 1,1-dioxide |
| 211 | 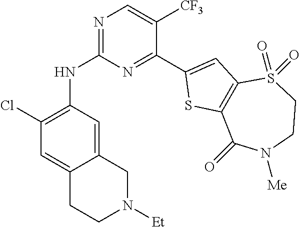 [SMILES: CCN1CCc2cc(Cl)c(Nc3ncc(C(F)(F)F)c(-c4cc5c(s4)C(=O)N(C)CCS5(=O)=O)n3)cc2C1] | 7-(2-((6-chloro-2-ethyl-1,2,3,4- tetrahydroisoquinolin-7-yl)amino)- 5-(trifluoromethyl)pyrimidin-4-yl)- 4-methyl-3,4-dihydrothieno[2,3- f][1,4]thiazepin-5(2H)-one 1,1- dioxide |
| 212 | 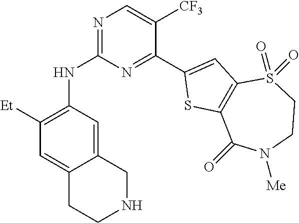 [SMILES: CCc1cc2c(cc1Nc1ncc(C(F)(F)F)c(-c3cc4c(s3)C(=O)N(C)CCS4(=O)=O)n1)CNCC2] | 7-(2-((6-ethyl-1,2,3,4- tetrahydroisoquinolin-7-yl)amino)- 5-(trifluoromethyl)pyrimidin-4-yl)- 4-methyl-3,4-dihydrothieno[2,3- f][1,4]thiazepin-5(2H)-one 1,1- dioxide |
| 213 | 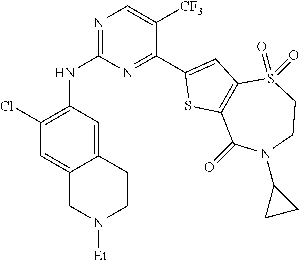 [SMILES: CCN1CCc2cc(Nc3ncc(C(F)(F)F)c(-c4cc5c(s4)C(=O)N(C4CC4)CCS5(=O)=O)n3)c(Cl)cc2C1] | 7-(2-((7-chloro-2-ethyl-1,2,3,4- tetrahydroisoquinolin-6-yl)amino)- 5-(trifluoromethyl)pyrimidin-4-yl)- 4-cyclopropyl-3,4- dihydrothieno[2,3-f][1,4]thiazepin- 5(2H)-one 1,1-dioxide |
| 214 | 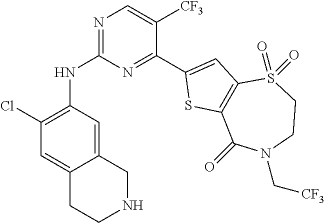 [SMILES: O=C1c2sc(-c3nc(Nc4cc5c(cc4Cl)CCNC5)ncc3C(F)(F)F)cc2S(=O)(=O)CCN1CC(F)(F)F] | 7-(2-((6-chloro-1,2,3,4- tetrahydroisoquinolin-7-yl)amino)- 5-(trifluoromethyl)pyrimidin-4-yl)- 4-(2,2,2-trifluoroethyl)-3,4- dihydrothieno[2,3-f][1,4]thiazepin- 5(2H)-one 1,1-dioxide |
| 215 | 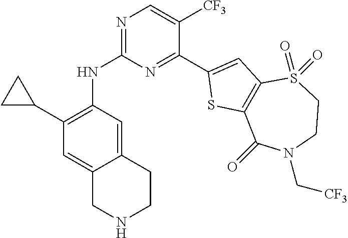 [SMILES: O=C1c2sc(-c3nc(Nc4cc5c(cc4C4CC4)CNCC5)ncc3C(F)(F)F)cc2S(=O)(=O)CCN1CC(F)(F)F] | 7-(2-((7-cyclopropyl-1,2,3,4- tetrahydroisoquinolin-6-yl)amino)- 5-(trifluoromethyl)pyrimidin-4-yl)- 4-(2,2,2-trifluoroethyl)-3,4- dihydrothieno[2,3-f][1,4]thiazepin- 5(2H)-one 1,1-dioxide |
| 216 | 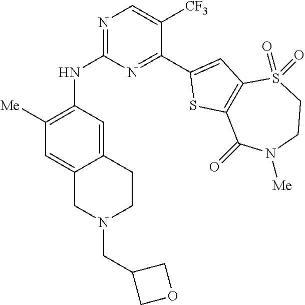 [SMILES: Cc1cc2c(cc1Nc1ncc(C(F)(F)F)c(-c3cc4c(s3)C(=O)N(C)CCS4(=O)=O)n1)CCN(CC1COC1)C2] | 4-methyl-7-(2-((7-methyl-2- (oxetan-3-ylmethyl)-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 217 | 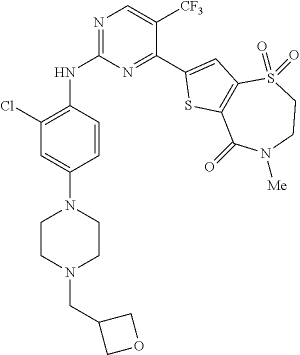 [SMILES: CN1CCS(=O)(=O)c2cc(-c3nc(Nc4ccc(N5CCN(CC6COC6)CC5)cc4Cl)ncc3C(F)(F)F)sc2C1=O] | 7-(2-((2-chloro-4-(4-(oxetan-3- ylmethyl)piperazin-1- yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 218 | 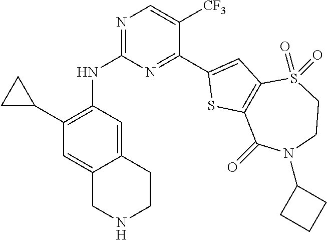 [SMILES: O=C1c2sc(-c3nc(Nc4cc5c(cc4C4CC4)CNCC5)ncc3C(F)(F)F)cc2S(=O)(=O)CCN1C1CCC1] | 4-cyclobutyl-7-(2-((7-cyclopropyl- 1,2,3,4-tetrahydroisoquinolin-6- yl)amino)-5- (trifluoromethyl)pyrimidin-4-yl)- 3,4-dihydrothieno[2,3- f][1,4]thiazepin-5(2H)-one 1,1- dioxide |
| 219 | 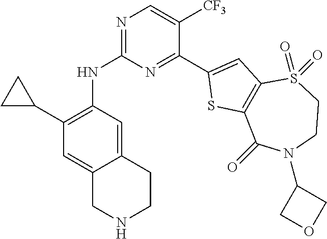 [SMILES: O=C1c2sc(-c3nc(Nc4cc5c(cc4C4CC4)CNCC5)ncc3C(F)(F)F)cc2S(=O)(=O)CCN1C1COC1] | 7-(2-((7-cyclopropyl-1,2,3,4- tetrahydroisoquinolin-6-yl)amino)- 5-(trifluoromethyl)pyrimidin-4-yl)- 4-(oxetan-3-yl)-3,4- dihydrothieno[2,3-f][1,4]thiazepin- 5(2H)-one 1,1-dioxide |
| 220 | 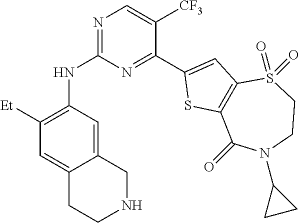 [SMILES: CCc1cc2c(cc1Nc1ncc(C(F)(F)F)c(-c3cc4c(s3)C(=O)N(C3CC3)CCS4(=O)=O)n1)CNCC2] | 4-cyclopropyl-7-(2-((6-ethyl- 1,2,3,4-tetrahydroisoquinolin-7- yl)amino)-5- (trifluoromethyl)pyrimidin-4-yl)- 3,4-dihydrothieno[2,3- f][1,4]thiazepin-5(2H)-one 1,1- dioxide |
| 221 | 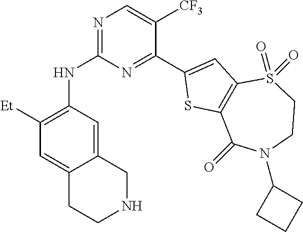 [SMILES: CCc1cc2c(cc1Nc1ncc(C(F)(F)F)c(-c3cc4c(s3)C(=O)N(C3CCC3)CCS4(=O)=O)n1)CNCC2] | 4-cyclobutyl-7-(2-((6-ethyl-1,2,3,4- tetrahydroisoquinolin-7-yl)amino)- 5-(trifluoromethyl)pyrimidin-4-yl)- 3,4-dihydrothieno[2,3- f][1,4]thiazepin-5(2H)-one 1,1- dioxide |
| 222 | 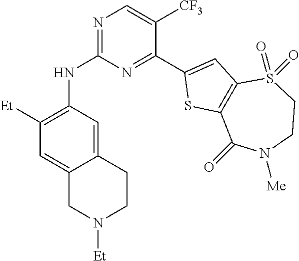 [SMILES: CCc1cc2c(cc1Nc1ncc(C(F)(F)F)c(-c3cc4c(s3)C(=O)N(C)CCS4(=O)=O)n1)CCN(CC)C2] | 7-(2-((2,7-diethyl-1,2,3,4- tetrahydroisoquinolin-6-yl)amino)- 5-(trifluoromethyl)pyrimidin-4-yl)- 4-methyl-3,4-dihydrothieno[2,3- f][1,4]thiazepin-5(2H)-one 1,1- dioxide |
| 223 | 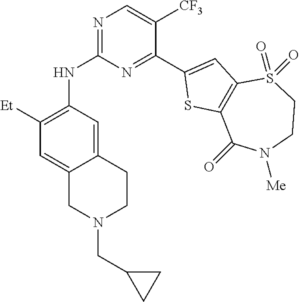 [SMILES: CCc1cc2c(cc1Nc1ncc(C(F)(F)F)c(-c3cc4c(s3)C(=O)N(C)CCS4(=O)=O)n1)CCN(CC1CC1)C2] | 7-(2-((2-(cyclopropylmethyl)-7- ethyl-1,2,3,4-tetrahydroisoquinolin- 6-yl)amino)-5- (trifluoromethyl)pyrimidin-4-yl)-4- methyl-3,4-dihydrothieno[2,3- f][1,4]thiazepin-5(2H)-one 1,1- dioxide |
| 224 | 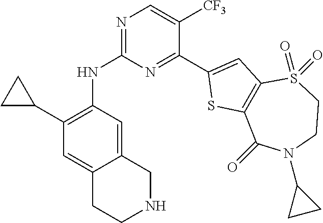 [SMILES: O=C1c2sc(-c3nc(Nc4cc5c(cc4C4CC4)CCNC5)ncc3C(F)(F)F)cc2S(=O)(=O)CCN1C1CC1] | 4-cyclopropyl-7-(2-((6- cyclopropyl-1,2,3,4- tetrahydroisoquinolin-7-yl)amino)- 5-(trifluoromethyl)pyrimidin-4-yl)- 3,4-dihydrothieno[2,3- f][1,4]thiazepin-5(2H)-one 1,1- dioxide |
| 225 | 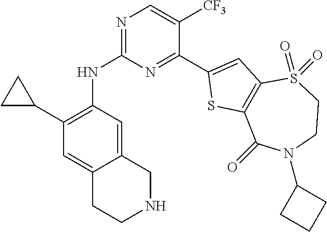 [SMILES: O=C1c2sc(-c3nc(Nc4cc5c(cc4C4CC4)CCNC5)ncc3C(F)(F)F)cc2S(=O)(=O)CCN1C1CCC1] | 4-cyclobutyl-7-(2-((6-cyclopropyl- 1,2,3,4-tetrahydroisoquinolin-7- yl)amino)-5- (trifluoromethyl)pyrimidin-4-yl)- 3,4-dihydrothieno[2,3- f][1,4]thiazepin-5(2H)-one 1,1- dioxide |
| 226 | 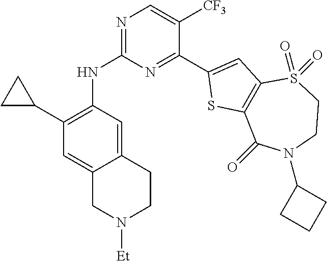 [SMILES: CCN1CCc2cc(Nc3ncc(C(F)(F)F)c(-c4cc5c(s4)C(=O)N(C4CCC4)CCS5(=O)=O)n3)c(C3CC3)cc2C1] | 4-cyclobutyl-7-(2-((7-cyclopropyl- 2-ethyl-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 227 | 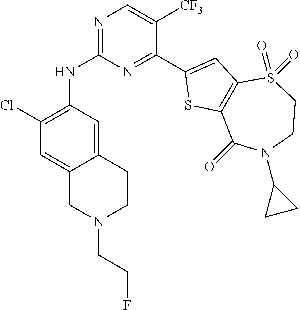 [SMILES: O=C1c2sc(-c3nc(Nc4cc5c(cc4Cl)CN(CCF)CC5)ncc3C(F)(F)F)cc2S(=O)(=O)CCN1C1CC1] | 7-(2-((7-chloro-2-(2-fluoroethyl)- 1,2,3,4-tetrahydroisoquinolin-6- yl)amino)-5- (trifluoromethyl)pyrimidin-4-yl)-4- cyclopropyl-3,4-dihydrothieno[2,3- f][1,4]thiazepin-5(2H)-one 1,1- dioxide |
| 228 | 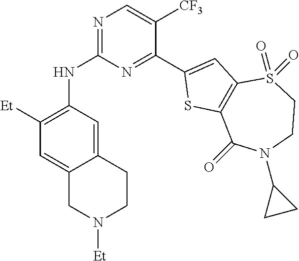 [SMILES: CCc1cc2c(cc1Nc1ncc(C(F)(F)F)c(-c3cc4c(s3)C(=O)N(C3CC3)CCS4(=O)=O)n1)CCN(CC)C2] | 4-cyclopropyl-7-(2-((2,7-diethyl- 1,2,3,4-tetrahydroisoquinolin-6- yl)amino)-5- (trifluoromethyl)pyrimidin-4-yl)- 3,4-dihydrothieno[2,3- f][1,4]thiazepin-5(2H)-one 1,1- dioxide |
| 229 | 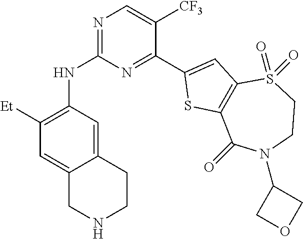 [SMILES: CCc1cc2c(cc1Nc1ncc(C(F)(F)F)c(-c3cc4c(s3)C(=O)N(C3COC3)CCS4(=O)=O)n1)CCNC2] | 7-(2-((7-ethyl-1,2,3,4- tetrahydroisoquinolin-6-yl)amino)- 5-(trifluoromethyl)pyrimidin-4-yl)- 4-(oxetan-3-yl)-3,4- dihydrothieno[2,3-f][1,4]thiazepin- 5(2H)-one 1,1-dioxide |
| 230 | 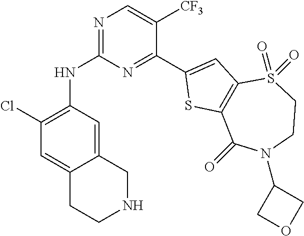 [SMILES: O=C1c2sc(-c3nc(Nc4cc5c(cc4Cl)CCNC5)ncc3C(F)(F)F)cc2S(=O)(=O)CCN1C1COC1] | 7-(2-((6-chloro-1,2,3,4- tetrahydroisoquinolin-7-yl)amino)- 5-(trifluoromethyl)pyrimidin-4-yl)- 4-(oxetan-3-yl)-3,4- dihydrothieno[2,3-f][1,4]thiazepin- 5(2H)-one 1,1-dioxide |
| 231 | 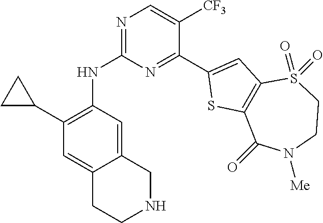 [SMILES: CN1CCS(=O)(=O)c2cc(-c3nc(Nc4cc5c(cc4C4CC4)CCNC5)ncc3C(F)(F)F)sc2C1=O] | 7-(2-((6-cyclopropyl-1,2,3,4- tetrahydroisoquinolin-7-yl)amino)- 5-(trifluoromethyl)pyrimidin-4-yl)- 4-methyl-3,4-dihydrothieno[2,3- f][1,4]thiazepin-5(2H)-one 1,1- dioxide |
| 232 | 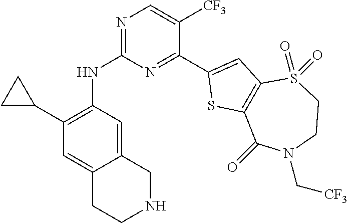 [SMILES: O=C1c2sc(-c3nc(Nc4cc5c(cc4C4CC4)CCNC5)ncc3C(F)(F)F)cc2S(=O)(=O)CCN1CC(F)(F)F] | 7-(2-((6-cyclopropyl-1,2,3,4- tetrahydroisoquinolin-7-yl)amino)- 5-(trifluoromethyl)pyrimidin-4-yl)- 4-(2,2,2-trifluoroethyl)-3,4- dihydrothieno[2,3-f][1,4]thiazepin- 5(2H)-one 1,1-dioxide |
| 233 | 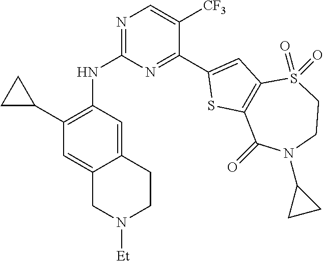 [SMILES: CCN1CCc2cc(Nc3ncc(C(F)(F)F)c(-c4cc5c(s4)C(=O)N(C4CC4)CCS5(=O)=O)n3)c(C3CC3)cc2C1] | 4-cyclopropyl-7-(2-((7- cyclopropyl-2-ethyl-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 234 | 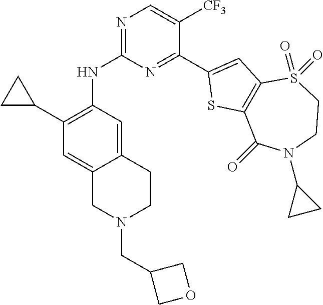 [SMILES: O=C1c2sc(-c3nc(Nc4cc5c(cc4C4CC4)CN(CC4COC4)CC5)ncc3C(F)(F)F)cc2S(=O)(=O)CCN1C1CC1] | 4-cyclopropyl-7-(2-((7- cyclopropyl-2-(oxetan-3-ylmethyl)- 1,2,3,4-tetrahydroisoquinolin-6- yl)amino)-5- (trifluoromethyl)pyrimidin-4-yl)- 3,4-dihydrothieno[2,3- f][1,4]thiazepin-5(2H)-one 1,1- dioxide |
| 235 | 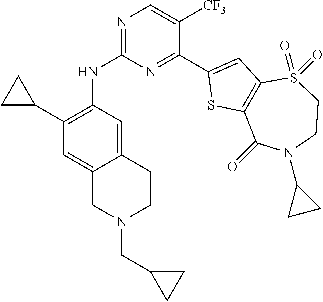 [SMILES: O=C1c2sc(-c3nc(Nc4cc5c(cc4C4CC4)CN(CC4CC4)CC5)ncc3C(F)(F)F)cc2S(=O)(=O)CCN1C1CC1] | 4-cyclopropyl-7-(2-((7- cyclopropyl-2- (cyclopropylmethyl)-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 236 | 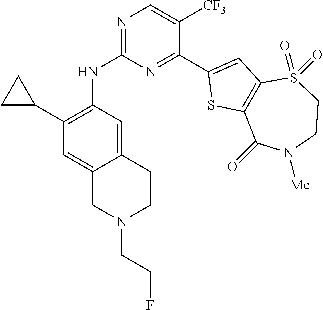 [SMILES: CN1CCS(=O)(=O)c2cc(-c3nc(Nc4cc5c(cc4C4CC4)CN(CCF)CC5)ncc3C(F)(F)F)sc2C1=O] | 7-(2-((7-cyclopropyl-2-(2- fluoroethyl)-1,2,3,4- tetrahydroisoquinolin-6-yl)amino)- 5-(trifluoromethyl)pyrimidin-4-yl)- 4-methyl-3,4-dihydrothieno[2,3- f][1,4]thiazepin-5(2H)-one 1,1- dioxide |
| 237 | 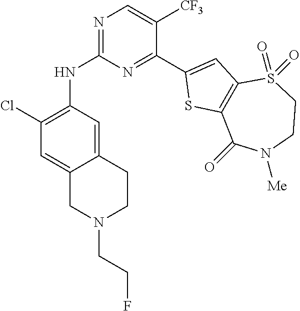 [SMILES: CN1CCS(=O)(=O)c2cc(-c3nc(Nc4cc5c(cc4Cl)CN(CCF)CC5)ncc3C(F)(F)F)sc2C1=O] | 7-(2-((7-chloro-2-(2-fluoroethyl)- 1,2,3,4-tetrahydroisoquinolin-6- yl)amino)-5- (trifluoromethyl)pyrimidin-4-yl)-4- methyl-3,4-dihydrothieno[2,3- f][1,4]thiazepin-5(2H)-one 1,1- dioxid |
| 238 | 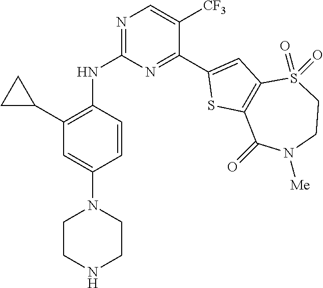 [SMILES: CN1CCS(=O)(=O)c2cc(-c3nc(Nc4ccc(N5CCNCC5)cc4C4CC4)ncc3C(F)(F)F)sc2C1=O] | 7-(2-((2-cyclopropyl-4-(piperazin- 1-yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 239 | 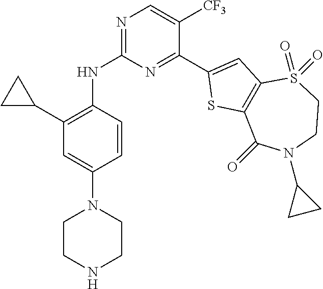 [SMILES: O=C1c2sc(-c3nc(Nc4ccc(N5CCNCC5)cc4C4CC4)ncc3C(F)(F)F)cc2S(=O)(=O)CCN1C1CC1] | 4-cyclopropyl-7-(2-((2- cyclopropyl-4-(piperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 240 | 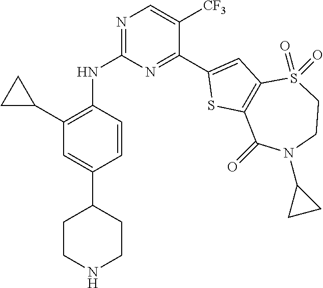 [SMILES: O=C1c2sc(-c3nc(Nc4ccc(C5CCNCC5)cc4C4CC4)ncc3C(F)(F)F)cc2S(=O)(=O)CCN1C1CC1] | 4-cyclopropyl-7-(2-((2- cyclopropyl-4-(piperidin-4- yl)phenyl)amino)-5- (trifluoromethyl)pyrimidin-4-yl)- 3,4-dihydrothieno[2,3- f][1,4]thiazepin-5(2H)-one 1,1- dioxide |
| 241 | 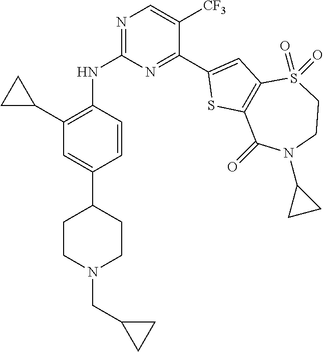 [SMILES: O=C1c2sc(-c3nc(Nc4ccc(C5CCN(CC6CC6)CC5)cc4C4CC4)ncc3C(F)(F)F)cc2S(=O)(=O)CCN1C1CC1] | 4-cyclopropyl-7-(2-((2- cyclopropyl-4-(1- (cyclopropylmethyl)piperidin-4- yl)phenyl)amino)-5- (trifluoromethyl)pyrimidin-4-yl)- 3,4-dihydrothieno[2,3- f][1,4]thiazepin-5(2H)-one 1,1- dioxide |
| 242 | 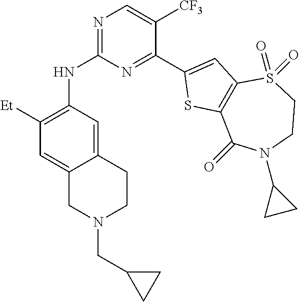 [SMILES: CCc1cc2c(cc1Nc1ncc(C(F)(F)F)c(-c3cc4c(s3)C(=O)N(C3CC3)CCS4(=O)=O)n1)CCN(CC1CC1)C2] | 4-cyclopropyl-7-(2-((2- (cyclopropylmethyl)-7-ethyl- 1,2,3,4-tetrahydroisoquinolin-6- yl)amino)-5- (trifluoromethyl)pyrimidin-4-yl)- 3,4-dihydrothieno[2,3- f][1,4]thiazepin-5(2H)-one 1,1- dioxide |
| 243 | 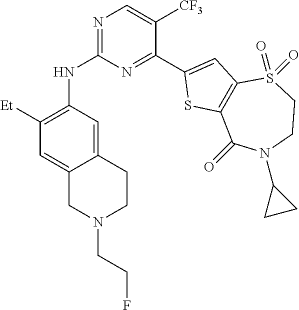 [SMILES: CCc1cc2c(cc1Nc1ncc(C(F)(F)F)c(-c3cc4c(s3)C(=O)N(C3CC3)CCS4(=O)=O)n1)CCN(CCF)C2] | 7-(2-((7-chloro-2-(2-fluoroethyl)- 1,2,3,4-tetrahydroisoquinolin-6- yl)amino)-5- (trifluoromethyl)pyrimidin-4-yl)-4- methyl-3,4-dihydrothieno[2,3- f][1,4]thiazepin-5(2H)-one 1,1- dioxide |
| 244 | 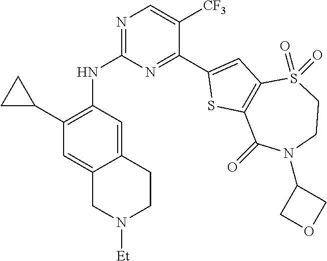 [SMILES: CCN1CCc2cc(Nc3ncc(C(F)(F)F)c(-c4cc5c(s4)C(=O)N(C4COC4)CCS5(=O)=O)n3)c(C3CC3)cc2C1] | 7-(2-((7-cyclopropyl-2-ethyl- 1,2,3,4-tetrahydroisoquinolin-6- yl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 245 | 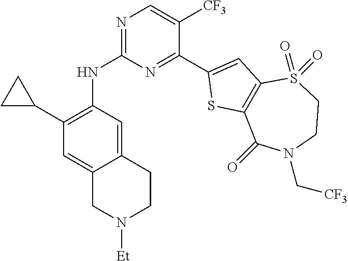 [SMILES: CCN1CCc2cc(Nc3ncc(C(F)(F)F)c(-c4cc5c(s4)C(=O)N(CC(F)(F)F)CCS5(=O)=O)n3)c(C3CC3)cc2C1] | 7-(2-((7-cyclopropyl-2-ethyl- 1,2,3,4-tetrahydroisoquinolin-6- yl)amino)-5- (trifluoromethyl)pyrimidin-4-yl)-4- (2,2,2-trifluoroethyl)-3,4- dihydrothieno[2,3-f][1,4]thiazepin- 5(2H)-one 1,1-dioxide |
| 246 | 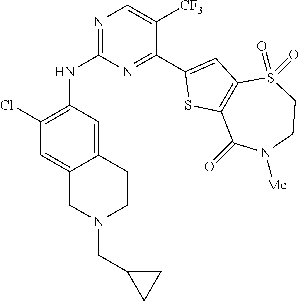 [SMILES: CN1CCS(=O)(=O)c2cc(-c3nc(Nc4cc5c(cc4Cl)CN(CC4CC4)CC5)ncc3C(F)(F)F)sc2C1=O] | 7-(2-((7-chloro-2- (cyclopropylmethyl)-1,2,3,4- tetrahydroisoquinolin-6-yl)amino)- 5-(trifluoromethyl)pyrimidin-4-yl)- 4-methyl-3,4-dihydrothieno[2,3- f][1,4]thiazepin-5(2H)-one 1,1- dioxide |
| 247 | 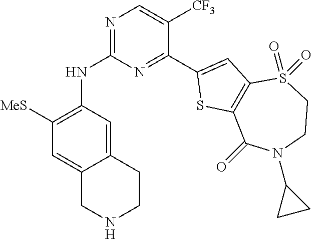 [SMILES: CSc1cc2c(cc1Nc1ncc(C(F)(F)F)c(-c3cc4c(s3)C(=O)N(C3CC3)CCS4(=O)=O)n1)CCNC2] | 4-cyclopropyl-7-(2-((7- (methylthio)-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 248 | 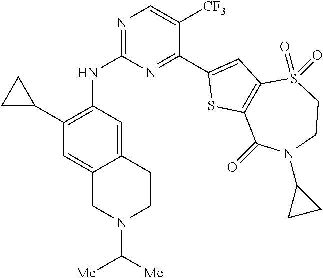 [SMILES: CC(C)N1CCc2cc(Nc3ncc(C(F)(F)F)c(-c4cc5c(s4)C(=O)N(C4CC4)CCS5(=O)=O)n3)c(C3CC3)cc2C1] | 4-cyclopropyl-7-(2-((7- cyclopropyl-2-isopropyl-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 249 | 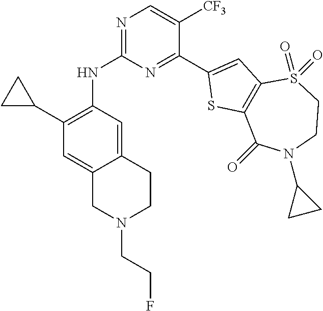 [SMILES: O=C1c2sc(-c3nc(Nc4cc5c(cc4C4CC4)CN(CCF)CC5)ncc3C(F)(F)F)cc2S(=O)(=O)CCN1C1CC1] | 4-cyclopropyl-7-(2-((7- cyclopropyl-2-(2-fluoroethyl)- 1,2,3,4-tetrahydroisoquinolin-6- yl)amino)-5- (trifluoromethyl)pyrimidin-4-yl)- 3,4-dihydrothieno[2,3- f][1,4]thiazepin-5(2H)-one 1,1- dioxide |
| 250 | 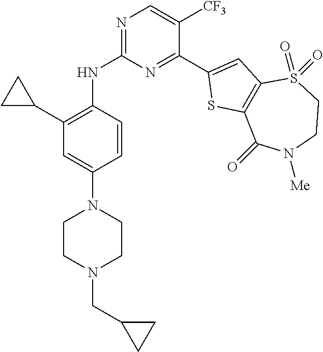 [SMILES: CN1CCS(=O)(=O)c2cc(-c3nc(Nc4ccc(N5CCN(CC6CC6)CC5)cc4C4CC4)ncc3C(F)(F)F)sc2C1=O] | 7-(2-((2-cyclopropyl-4-(4- (cyclopropylmethyl)piperazin-1- yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 251 | 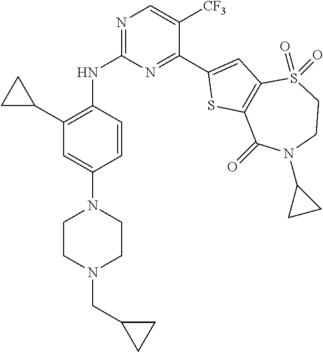 [SMILES: O=C1c2sc(-c3nc(Nc4ccc(N5CCN(CC6CC6)CC5)cc4C4CC4)ncc3C(F)(F)F)cc2S(=O)(=O)CCN1C1CC1] | 4-cyclopropyl-7-(2-((2- cyclopropyl-4-(4- (cyclopropylmethyl)piperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 252 | 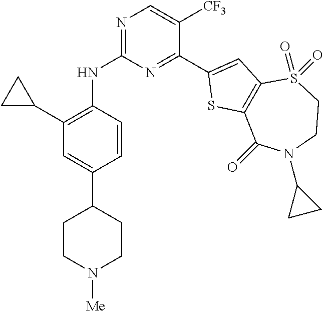 [SMILES: CN1CCC(c2ccc(Nc3ncc(C(F)(F)F)c(-c4cc5c(s4)C(=O)N(C4CC4)CCS5(=O)=O)n3)c(C3CC3)c2)CC1] | 4-cyclopropyl-7-(2-((2- cyclopropyl-4-(1-methylpiperidin- 4-yl)phenyl)amino)-5- (trifluoromethyl)pyrimidin-4-yl)- 3,4-dihydrothieno[2,3- f][1,4]thiazepin-5(2H)-one 1,1- dioxide |
| 253 | 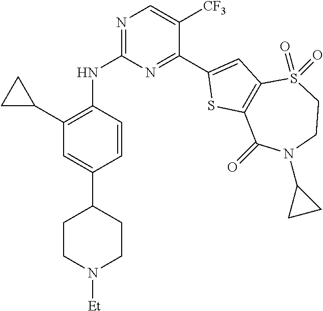 [SMILES: CCN1CCC(c2ccc(Nc3ncc(C(F)(F)F)c(-c4cc5c(s4)C(=O)N(C4CC4)CCS5(=O)=O)n3)c(C3CC3)c2)CC1] | 4-cyclopropyl-7-(2-((2- cyclopropyl-4-(1-ethylpiperidin-4- yl)phenyl)amino)-5- (trifluoromethyl)pyrimidin-4-yl)- 3,4-dihydrothieno[2,3- f][1,4]thiazepin-5(2H)-one 1,1- dioxide |
| 254 | 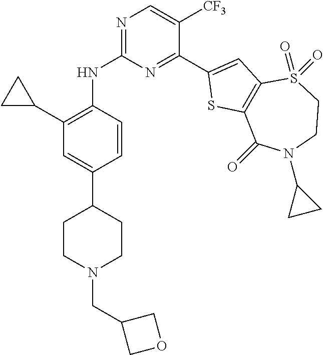 [SMILES: O=C1c2sc(-c3nc(Nc4ccc(C5CCN(CC6COC6)CC5)cc4C4CC4)ncc3C(F)(F)F)cc2S(=O)(=O)CCN1C1CC1] | 4-cyclopropyl-7-(2-((2- cyclopropyl-4-(1-(oxetan-3- ylmethyl)piperidin-4- yl)phenyl)amino)-5- (trifluoromethyl)pyrimidin-4-yl)- 3,4-dihydrothieno[2,3- f][1,4]thiazepin-5(2H)-one 1,1- dioxide |
| 255 | 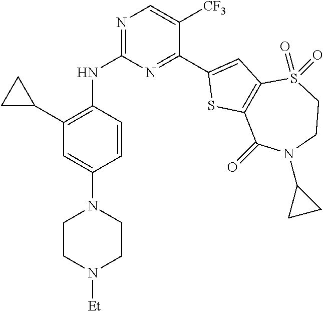 [SMILES: CCN1CCN(c2ccc(Nc3ncc(C(F)(F)F)c(-c4cc5c(s4)C(=O)N(C4CC4)CCS5(=O)=O)n3)c(C3CC3)c2)CC1] | 4-cyclopropyl-7-(2-((2- cyclopropyl-4-(4-ethylpiperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 256 | 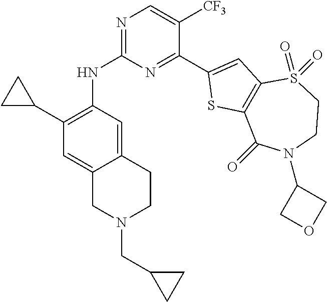 [SMILES: O=C1c2sc(-c3nc(Nc4cc5c(cc4C4CC4)CN(CC4CC4)CC5)ncc3C(F)(F)F)cc2S(=O)(=O)CCN1C1COC1] | 7-(2-((7-cyclopropyl-2- (cyclopropylmethyl)-1,2,3,4- tetrahydroisoquinolin-6-yl)amino)- 5-(trifluoromethyl)pyrimidin-4-yl)- 4-(oxetan-3-yl)-3,4- dihydrothieno[2,3-f][1,4]thiazepin- 5(2H)-one 1,1-dioxide |
| 257 | 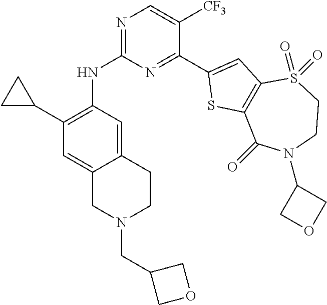 [SMILES: O=C1c2sc(-c3nc(Nc4cc5c(cc4C4CC4)CN(CC4COC4)CC5)ncc3C(F)(F)F)cc2S(=O)(=O)CCN1C1COC1] | 7-(2-((7-cyclopropyl-2-(oxetan-3- ylmethyl)-1,2,3,4- tetrahydroisoquinolin-6-yl)amino)- 5-(trifluoromethyl)pyrimidin-4-yl)- 4-(oxetan-3-yl)-3,4- dihydrothieno[2,3-f][1,4]thiazepin- 5(2H)-one 1,1-dioxide |
| 258 | 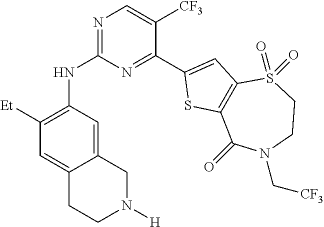 [SMILES: CCc1cc2c(cc1Nc1ncc(C(F)(F)F)c(-c3cc4c(s3)C(=O)N(CC(F)(F)F)CCS4(=O)=O)n1)CNCC2] | 7-(2-((6-ethyl-1,2,3,4- tetrahydroisoquinolin-7-yl)amino)- 5-(trifluoromethyl)pyrimidin-4-yl)- 4-(2,2,2-trifluoroethyl)-3,4- dihydrothieno[2,3-f][1,4]thiazepin- 5(2H)-one 1,1-dioxide |
| 259 | 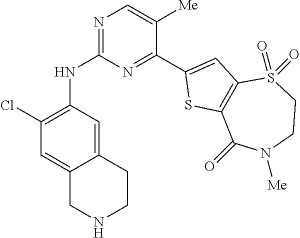 [SMILES: Cc1cnc(Nc2cc3c(cc2Cl)CNCC3)nc1-c1cc2c(s1)C(=O)N(C)CCS2(=O)=O] | 7-(2-((7-chloro-1,2,3,4- tetrahydroisoquinolin-6-yl)amino)- 5-methylpyrimidin-4-yl)-4-methyl- 3,4-dihydrothieno[2,3- f][1,4]thiazepin-5(2H)-one 1,1- dioxide |
| 260 | 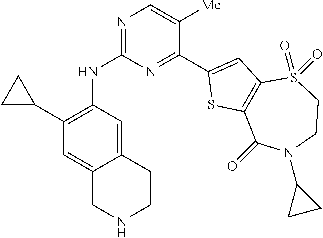 [SMILES: Cc1cnc(Nc2cc3c(cc2C2CC2)CNCC3)nc1-c1cc2c(s1)C(=O)N(C1CC1)CCS2(=O)=O] | 4-cyclopropyl-7-(2-((7- cyclopropyl-1,2,3,4- tetrahydroisoquinolin-6-yl)amino)- 5-methylpyrimidin-4-y1)-3,4- dihydrothieno[2,3-f][1,4]thiazepin- 5(2H)-one 1,1-dioxide |
| 261 | 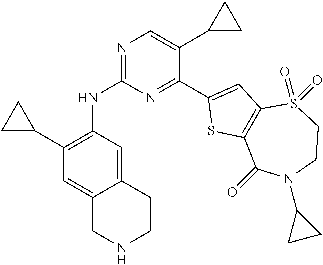 [SMILES: O=C1c2sc(-c3nc(Nc4cc5c(cc4C4CC4)CNCC5)ncc3C3CC3)cc2S(=O)(=O)CCN1C1CC1] | 4-cyclopropyl-7-(5-cyclopropyl-2- ((7-cyclopropyl-1,2,3,4- tetrahydroisoquinolin-6- yl)amino)pyrimidin-4-yl)-3,4- dihydrothieno[2,3-f][1,4]thiazepin- 5(2H)-one 1,1-dioxide |
| 262 | 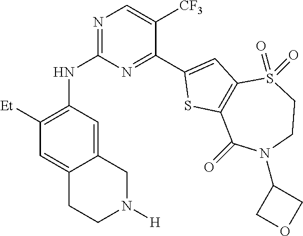 [SMILES: CCc1cc2c(cc1Nc1ncc(C(F)(F)F)c(-c3cc4c(s3)C(=O)N(C3COC3)CCS4(=O)=O)n1)CNCC2] | 7-(2-((6-ethyl-1,2,3,4- tetrahydroisoquinolin-7-yl)amino)- 5-(trifluoromethyl)pyrimidin-4-yl)- 4-(oxetan-3-yl)-3,4- dihydrothieno[2,3-f][1,4]thiazepin- 5(2H)-one 1,1-dioxide |
| 263 | 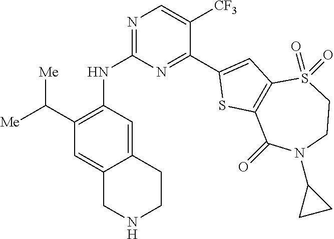 [SMILES: CC(C)c1cc2c(cc1Nc1ncc(C(F)(F)F)c(-c3cc4c(s3)C(=O)N(C3CC3)CCS4(=O)=O)n1)CCNC2] | 4-cyclopropyl-7-(2-((7-isopropyl- 1,2,3,4-tetrahydroisoquinolin-6- yl)amino)-5- (trifluoromethyl)pyrimidin-4-yl)- 3,4-dihydrothieno[2,3- f][1,4]thiazepin-5(2H)-one 1,1- dioxide |
| 264 | 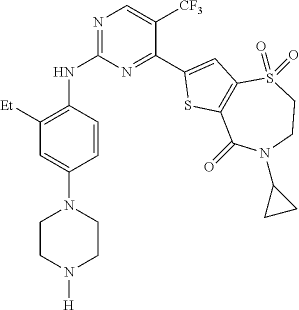 [SMILES: CCc1cc(N2CCNCC2)ccc1Nc1ncc(C(F)(F)F)c(-c2cc3c(s2)C(=O)N(C2CC2)CCS3(=O)=O)n1] | 4-cyclopropyl-7-(2-((2-ethyl-4- (piperazin-1-yl)phenyl)amino)-5- (trifluoromethyl)pyrimidin-4-yl)- 3,4-dihydrothieno[2,3- f][1,4]thiazepin-5(2H)-one 1,1- dioxide |
| 265 | 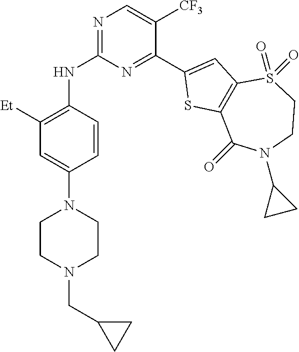 [SMILES: CCc1cc(N2CCN(CC3CC3)CC2)ccc1Nc1ncc(C(F)(F)F)c(-c2cc3c(s2)C(=O)N(C2CC2)CCS3(=O)=O)n1] | 4-cyclopropyl-7-(2-((4-(4- (cyclopropylmethyl)piperazin-1- yl)-2-ethylphenyl)amino)-5- (trifluoromethyl)pyrimidin-4-yl)- 3,4-dihydrothieno[2,3- f][1,4]thiazepin-5(2H)-one 1,1- dioxide |
| 266 | 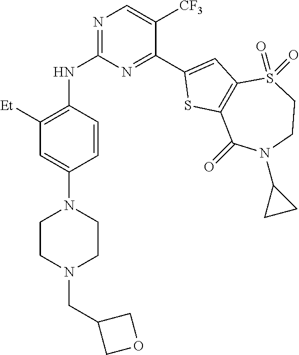 [SMILES: CCc1cc(N2CCN(CC3COC3)CC2)ccc1Nc1ncc(C(F)(F)F)c(-c2cc3c(s2)C(=O)N(C2CC2)CCS3(=O)=O)n1] | 4-cyclopropyl-7-(2-((2-ethyl-4-(4- (oxetan-3-ylmethyl)piperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 267 | 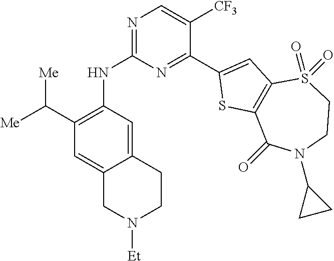 [SMILES: CCN1CCc2cc(Nc3ncc(C(F)(F)F)c(-c4cc5c(s4)C(=O)N(C4CC4)CCS5(=O)=O)n3)c(C(C)C)cc2C1] | 4-cyclopropyl-7-(2-((2-ethyl-7- isopropyl-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 268 | 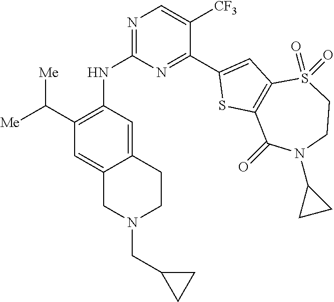 [SMILES: CC(C)c1cc2c(cc1Nc1ncc(C(F)(F)F)c(-c3cc4c(s3)C(=O)N(C3CC3)CCS4(=O)=O)n1)CCN(CC1CC1)C2] | 4-cyclopropyl-7-(2-((2- (cyclopropylmethyl)-7-isopropyl- 1,2,3,4-tetrahydroisoquinolin-6- yl)amino)-5- (trifluoromethyl)pyrimidin-4-yl)- 3,4-dihydrothieno[2,3- f][1,4]thiazepin-5(2H)-one 1,1- dioxide |
| 269 | 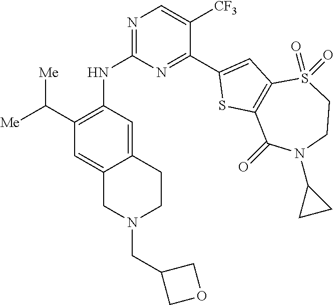 [SMILES: CC(C)c1cc2c(cc1Nc1ncc(C(F)(F)F)c(-c3cc4c(s3)C(=O)N(C3CC3)CCS4(=O)=O)n1)CCN(CC1COC1)C2] | 4-cyclopropyl-7-(2-((7-isopropyl-2- (oxetan-3-ylmethyl)-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 270 | 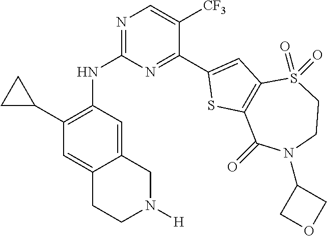 [SMILES: O=C1c2sc(-c3nc(Nc4cc5c(cc4C4CC4)CCNC5)ncc3C(F)(F)F)cc2S(=O)(=O)CCN1C1COC1] | 7-(2-((6-cyclopropyl-1,2,3,4- tetrahydroisoquinolin-7-yl)amino)- 5-(trifluoromethyl)pyrimidin-4-yl)- 4-(oxetan-3-yl)-3,4- dihydrothieno[2,3-f][1,4]thiazepin- 5(2H)-one 1,1-dioxide |
| 271 | 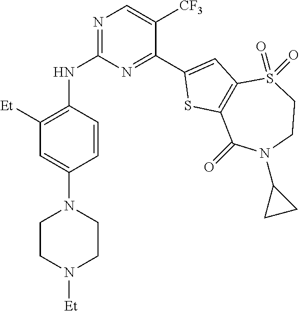 [SMILES: CCc1cc(N2CCN(CC)CC2)ccc1Nc1ncc(C(F)(F)F)c(-c2cc3c(s2)C(=O)N(C2CC2)CCS3(=O)=O)n1] | 4-cyclopropyl-7-(2-((2-ethyl-4-(4- ethylpiperazin-1-yl)phenyl)amino)- 5-(trifluoromethyl)pyrimidin-4-yl)- 3,4-dihydrothieno[2,3- f][1,4]thiazepin-5(2H)-one 1,1- dioxide |
| 272 | 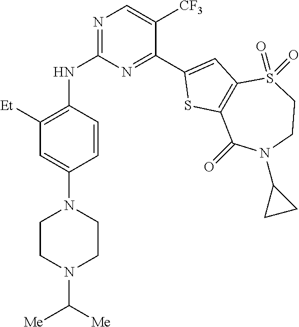 [SMILES: CCc1cc(N2CCN(C(C)C)CC2)ccc1Nc1ncc(C(F)(F)F)c(-c2cc3c(s2)C(=O)N(C2CC2)CCS3(=O)=O)n1] | 4-cyclopropyl-7-(2-((2-ethyl-4-(4- isopropylpiperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 273 | 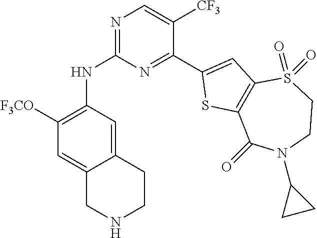 [SMILES: O=C1c2sc(-c3nc(Nc4cc5c(cc4OC(F)(F)F)CNCC5)ncc3C(F)(F)F)cc2S(=O)(=O)CCN1C1CC1] | 4-cyclopropyl-7-(2-((7- (trifluoromethoxy)-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 274 | 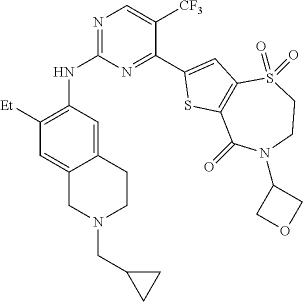 [SMILES: CCc1cc2c(cc1Nc1ncc(C(F)(F)F)c(-c3cc4c(s3)C(=O)N(C3COC3)CCS4(=O)=O)n1)CCN(CC1CC1)C2] | 7-(2-((2-(cyclopropylmethyl)-7- ethyl-1,2,3,4-tetrahydroisoquinolin- 6-yl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 275 | 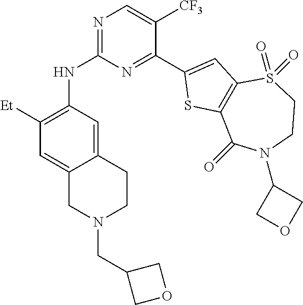 [SMILES: CCc1cc2c(cc1Nc1ncc(C(F)(F)F)c(-c3cc4c(s3)C(=O)N(C3COC3)CCS4(=O)=O)n1)CCN(CC1COC1)C2] | 7-(2-((7-ethyl-2-(oxetan-3- ylmethyl)-1,2,3,4- tetrahydroisoquinolin-6-yl)amino)- 5-(trifluoromethyl)pyrimidin-4-yl)- 4-(oxetan-3-y1)-3,4- dihydrothieno[2,3-f][1,4]thiazepin- 5(2H)-one 1,1-dioxide |
| 276 | 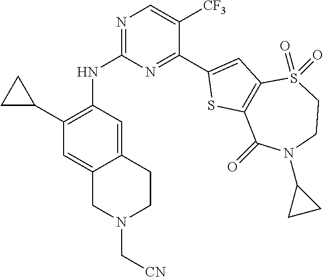 [SMILES: N#CCN1CCc2cc(Nc3ncc(C(F)(F)F)c(-c4cc5c(s4)C(=O)N(C4CC4)CCS5(=O)=O)n3)c(C3CC3)cc2C1] | 2-(7-cyclopropyl-6-((4-(4- cyclopropyl-1,1-dioxido-5-oxo- 2,3,4,5-tetrahydrothieno[2,3- f][1,4]thiazepin-7-yl)-5- (trifluoromethyl)pyrimidin-2- yl)amino)-3,4-dihydroisoquinolin- 2(1H)-yl)acetonitrile |
| 277 | 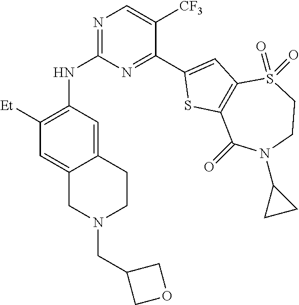 [SMILES: CCc1cc2c(cc1Nc1ncc(C(F)(F)F)c(-c3cc4c(s3)C(=O)N(C3CC3)CCS4(=O)=O)n1)CCN(CC1COC1)C2] | 4-cyclopropyl-7-(2-((7-ethyl-2- (oxetan-3-ylmethyl)-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 278 | 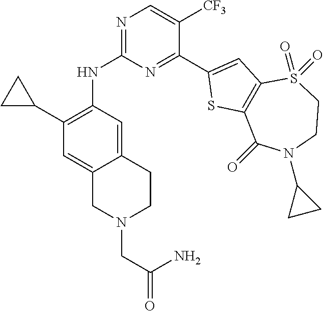 [SMILES: NC(=O)CN1CCc2cc(Nc3ncc(C(F)(F)F)c(-c4cc5c(s4)C(=O)N(C4CC4)CCS5(=O)=O)n3)c(C3CC3)cc2C1] | 2-(7-cyclopropyl-6-((4-(4- cyclopropyl-1,1-dioxido-5-oxo- 2,3,4,5-tetrahydrothieno[2,3- f][1,4]thiazepin-7-yl)-5- (trifluoromethyl)pyrimidin-2- yl)amino)-3,4-dihydroisoquinolin- 2(1H)-yl)acetamide |
| 279 | 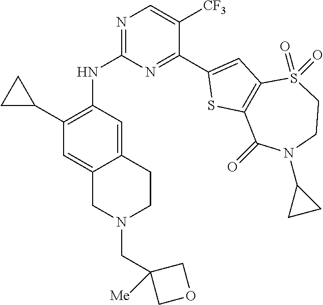 [SMILES: CC1(CN2CCc3cc(Nc4ncc(C(F)(F)F)c(-c5cc6c(s5)C(=O)N(C5CC5)CCS6(=O)=O)n4)c(C4CC4)cc3C2)COC1] | 4-cyclopropyl-7-(2-((7- cyclopropyl-2-((3-methyloxetan-3- yl)methyl)-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 280 | 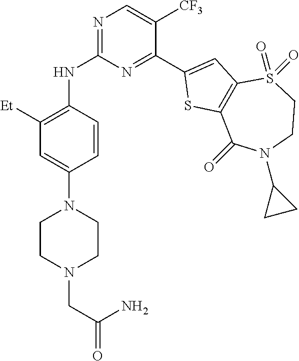 [SMILES: CCc1cc(N2CCN(CC(N)=O)CC2)ccc1Nc1ncc(C(F)(F)F)c(-c2cc3c(s2)C(=O)N(C2CC2)CCS3(=O)=O)n1] | 2-(4-(4-((4-(4-cyclopropyl-1,1- dioxido-5-oxo-2,3,4,5- tetrahydrothieno[2,3- f][1,4]thiazepin-7-y1)-5- (trifluoromethyl)pyrimidin-2- yl)amino)-3-ethylphenyl)piperazin- 1-yl)acetamide |
| 281 | 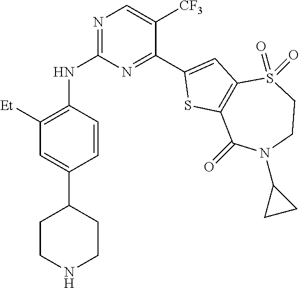 [SMILES: CCc1cc(C2CCNCC2)ccc1Nc1ncc(C(F)(F)F)c(-c2cc3c(s2)C(=O)N(C2CC2)CCS3(=O)=O)n1] | 4-cyclopropyl-7-(2-((2-ethyl-4- (piperidin-4-yl)phenyl)amino)-5- (trifluoromethyl)pyrimidin-4-yl)- 3,4-dihydrothieno[2,3- f][1,4]thiazepin-5(2H)-one 1,1- dioxide |
| 282 | 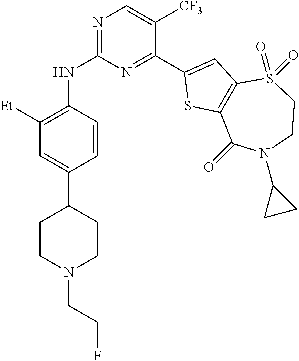 [SMILES: CCc1cc(C2CCN(CCF)CC2)ccc1Nc1ncc(C(F)(F)F)c(-c2cc3c(s2)C(=O)N(C2CC2)CCS3(=O)=O)n1] | 4-cyclopropyl-7-(2-((2-ethyl-4-(1- (2-fluoroethyl)piperidin-4- yl)phenyl)amino)-5- (trifluoromethyl)pyrimidin-4-yl)- 3,4-dihydrothieno[2,3- f][1,4]thiazepin-5(2H)-one 1,1- dioxide |
| 283 | 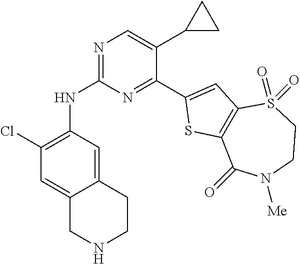 [SMILES: CN1CCS(=O)(=O)c2cc(-c3nc(Nc4cc5c(cc4Cl)CNCC5)ncc3C3CC3)sc2C1=O] | 7-(2-((7-chloro-1,2,3,4- tetrahydroisoquinolin-6-yl)amino)- 5-cyclopropylpyrimidin-4-yl)-4- methyl-3,4-dihydrothieno[2,3- f][1,4]thiazepin-5(2H)-one 1,1- dioxide |
| 284 | 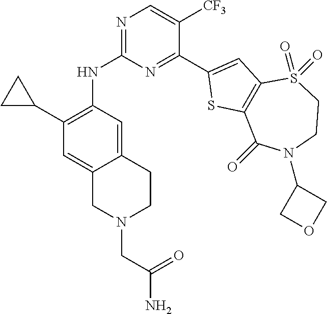 [SMILES: NC(=O)CN1CCc2cc(Nc3ncc(C(F)(F)F)c(-c4cc5c(s4)C(=O)N(C4COC4)CCS5(=O)=O)n3)c(C3CC3)cc2C1] | 2-(7-cyclopropyl-6-((4-(4-(oxetan- 3-yl)-1,1-dioxido-5-oxo-2,3,4,5- tetrahydrothieno[2,3- f][1,4]thiazepin-7-yl)-5- (trifluoromethyl)pyrimidin-2- yl)amino)-3,4-dihydroisoquinolin- 2(1H)-yl)acetamide |
| 285 | 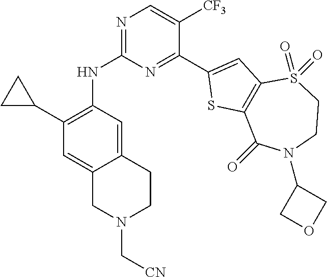 [SMILES: N#CCN1CCc2cc(Nc3ncc(C(F)(F)F)c(-c4cc5c(s4)C(=O)N(C4COC4)CCS5(=O)=O)n3)c(C3CC3)cc2C1] | 2-(7-cyclopropyl-6-((4-(4-(oxetan- 3-yl)-1,1-dioxido-5-oxo-2,3,4,5- tetrahydrothieno[2,3- f][1,4]thiazepin-7-yl)-5- (trifluoromethyl)pyrimidin-2- yl)amino)-3,4-dihydroisoquinolin- 2(1H)-yl)acetonitrile |
| 286 | 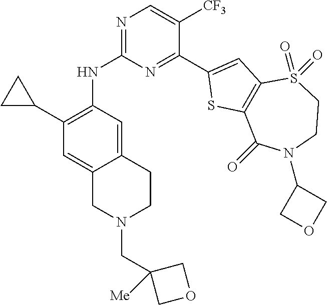 [SMILES: CC1(CN2CCc3cc(Nc4ncc(C(F)(F)F)c(-c5cc6c(s5)C(=O)N(C5COC5)CCS6(=O)=O)n4)c(C4CC4)cc3C2)COC1] | 7-(2-((7-cyclopropyl-2-((3- methyloxetan-3-yl)methyl)-1,2,3,4- tetrahydroisoquinolin-6-yl)amino)- 5-(trifluoromethyl)pyrimidin-4-yl)- 4-(oxetan-3-yl)-3,4- dihydrothieno[2,3-f][1,4]thiazepin- 5(2H)-one 1,1-dioxide |
| 287 | 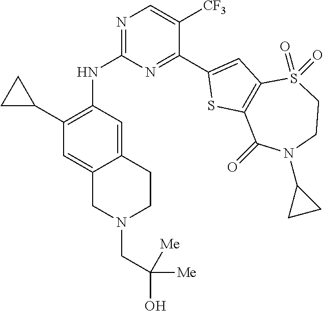 [SMILES: CC(C)(O)CN1CCc2cc(Nc3ncc(C(F)(F)F)c(-c4cc5c(s4)C(=O)N(C4CC4)CCS5(=O)=O)n3)c(C3CC3)cc2C1] | 4-cyclopropyl-7-(2-((7- cyclopropyl-2-(2-hydroxy-2- methylpropyl)-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 288 | 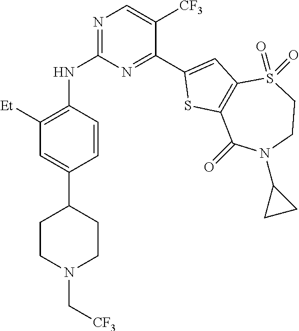 [SMILES: CCc1cc(C2CCN(CC(F)(F)F)CC2)ccc1Nc1ncc(C(F)(F)F)c(-c2cc3c(s2)C(=O)N(C2CC2)CCS3(=O)=O)n1] | 4-cyclopropyl-7-(2-((2-ethyl-4-(1- (2,2,2-trifluoroethyl)piperidin-4- yl)phenyl)amino)-5- (trifluoromethyl)pyrimidin-4-yl)- 3,4-dihydrothieno[2,3- f][1,4]thiazepin-5(2H)-one 1,1- dioxide |
| 289 | 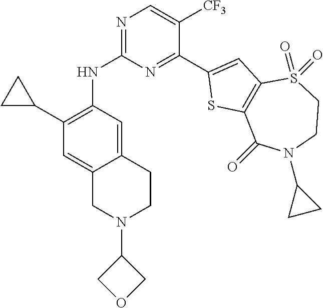 [SMILES: O=C1c2sc(-c3nc(Nc4cc5c(cc4C4CC4)CN(C4COC4)CC5)ncc3C(F)(F)F)cc2S(=O)(=O)CCN1C1CC1] | 4-cyclopropyl-7-(2-((7- cyclopropyl-2-(oxetan-3-yl)- 1,2,3,4-tetrahydroisoquinolin-6- yl)amino)-5- (trifluoromethyl)pyrimidin-4-yl)- 3,4-dihydrothieno[2,3- f][1,4]thiazepin-5(2H)-one 1,1- dioxide |
| 290 | 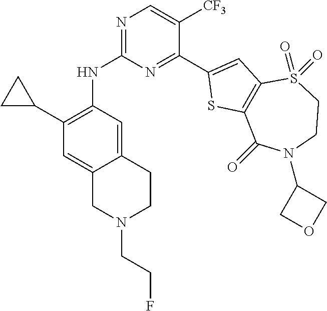 [SMILES: O=C1c2sc(-c3nc(Nc4cc5c(cc4C4CC4)CN(CCF)CC5)ncc3C(F)(F)F)cc2S(=O)(=O)CCN1C1COC1] | 7-(2-((7-cyclopropyl-2-(2- fluoroethyl)-1,2,3,4- tetrahydroisoquinolin-6-yl)amino)- 5-(trifluoromethyl)pyrimidin-4-yl)- 4-(oxetan-3-yl)-3,4- dihydrothieno[2,3-f][1,4]thiazepin- 5(2H)-one 1,1-dioxide |
| 291 | 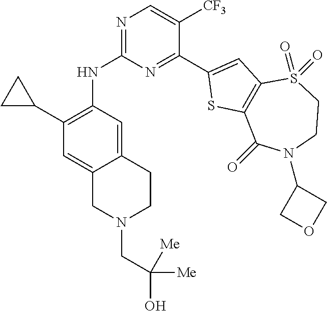 [SMILES: CC(C)(O)CN1CCc2cc(Nc3ncc(C(F)(F)F)c(-c4cc5c(s4)C(=O)N(C4COC4)CCS5(=O)=O)n3)c(C3CC3)cc2C1] | 7-(2-((7-cyclopropyl-2-(2-hydroxy- 2-methylpropyl)-1,2,3,4- tetrahydroisoquinolin-6-yl)amino)- 5-(trifluoromethyl)pyrimidin-4-yl)- 4-(oxetan-3-yl)-3,4- dihydrothieno[2,3-f][1,4]thiazepin- 5(2H)-one 1,1-dioxide |
| 292 | 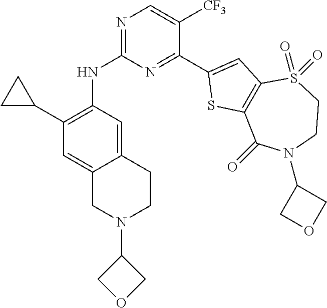 [SMILES: O=C1c2sc(-c3nc(Nc4cc5c(cc4C4CC4)CN(C4COC4)CC5)ncc3C(F)(F)F)cc2S(=O)(=O)CCN1C1COC1] | 7-(2-((7-cyclopropyl-2-(oxetan-3- yl)-1,2,3,4-tetrahydroisoquinolin-6- yl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 293 | 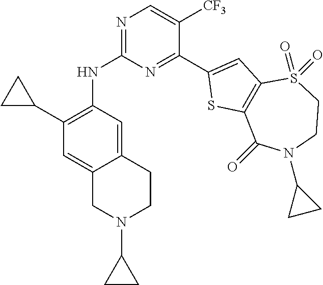 [SMILES: O=C1c2sc(-c3nc(Nc4cc5c(cc4C4CC4)CN(C4CC4)CC5)ncc3C(F)(F)F)cc2S(=O)(=O)CCN1C1CC1] | 4-Cyclopropyl-7-(2-((2,7- dicyclopropyl-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 294 | 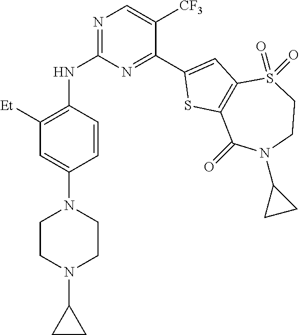 [SMILES: CCc1cc(N2CCN(C3CC3)CC2)ccc1Nc1ncc(C(F)(F)F)c(-c2cc3c(s2)C(=O)N(C2CC2)CCS3(=O)=O)n1] | 4-Cyclopropyl-7-(2-((4-(4- cyclopropylpiperazin-1-yl)-2- ethylphenyl)amino)-5- (trifluoromethyl)pyrimidin-4-yl)- 3,4-dihydrothieno[2,3- f][1,4]thiazepin-5(2H)-one 1,1- dioxide |
| 295 | 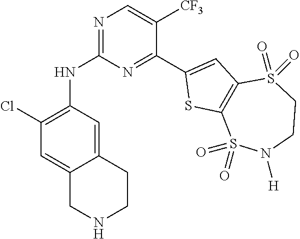 [SMILES: O=S1(=O)CCNS(=O)(=O)c2sc(-c3nc(Nc4cc5c(cc4Cl)CNCC5)ncc3C(F)(F)F)cc21] | 7-(2-((7-chloro-1,2,3,4- tetrahydroisoquinolin-6-yl)amino)- 5-(trifluoromethyl)pyrimidin-4-yl)- 3,4-dihydro-2H-thieno[3,2- f][1,5,2]dithiazepine 1,1,5,5- tetraoxide |
| 296 | 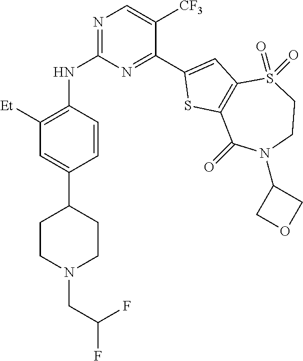 [SMILES: CCc1cc(C2CCN(CC(F)F)CC2)ccc1Nc1ncc(C(F)(F)F)c(-c2cc3c(s2)C(=O)N(C2COC2)CCS3(=O)=O)n1] | 7-(2-((4-(1-(2,2- difluoroethyl)piperidin-4-yl)-2- ethylphenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 297 | 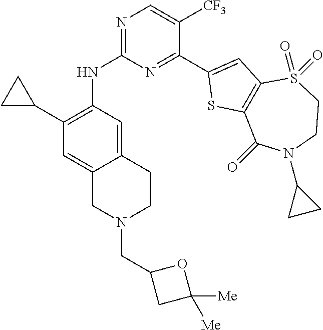 [SMILES: CC1(C)CC(CN2CCc3cc(Nc4ncc(C(F)(F)F)c(-c5cc6c(s5)C(=O)N(C5CC5)CCS6(=O)=O)n4)c(C4CC4)cc3C2)O1] | 4-Cyclopropyl-7-(2-((7- cyclopropyl-2-((4,4- dimethyloxetan-2-yl)methyl)- 1,2,3,4-tetrahydroisoquinolin-6- yl)amino)-5- (trifluoromethyl)pyrimidin-4-yl)- 3,4-dihydrothieno[2,3- f][1,4]thiazepin-5(2H)-one 1,1- dioxide |
| 298 | 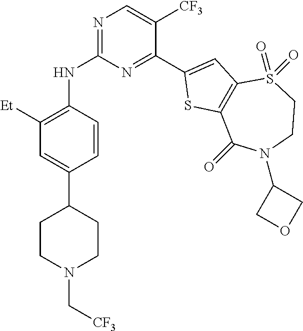 [SMILES: CCc1cc(C2CCN(CC(F)(F)F)CC2)ccc1Nc1ncc(C(F)(F)F)c(-c2cc3c(s2)C(=O)N(C2COC2)CCS3(=O)=O)n1] | 7-(2-((2-Ethyl-4-(1-(2,2,2- trifluoroethyl)piperidin-4- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 299 | 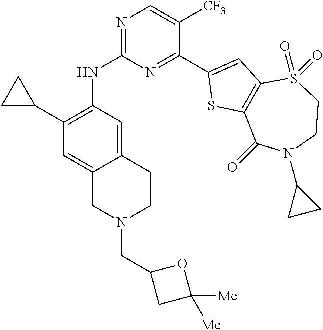 [SMILES: CC1(C)CC(CN2CCc3cc(Nc4ncc(C(F)(F)F)c(-c5cc6c(s5)C(=O)N(C5CC5)CCS6(=O)=O)n4)c(C4CC4)cc3C2)O1] | 4-cyclopropyl-7-(2-((7- cyclopropyl-2-(2-fluoro-2- methylpropyl)-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 300 | 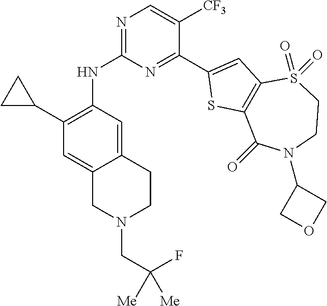 [SMILES: CC(C)(F)CN1CCc2cc(Nc3ncc(C(F)(F)F)c(-c4cc5c(s4)C(=O)N(C4COC4)CCS5(=O)=O)n3)c(C3CC3)cc2C1] | 7-(2-((7-cyclopropyl-2-(2-fluoro-2- methylpropyl)-1,2,3,4- tetrahydroisoquinolin-6-yl)amino)- 5-(trifluoromethyl)pyrimidin-4-yl)- 4-(oxetan-3-yl)-3,4- dihydrothieno[2,3-f][1,4]thiazepin- 5(2H)-one 1,1-dioxide |
| 301 | 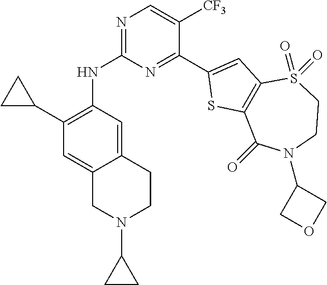 [SMILES: O=C1c2sc(-c3nc(Nc4cc5c(cc4C4CC4)CN(C4CC4)CC5)ncc3C(F)(F)F)cc2S(=O)(=O)CCN1C1COC1] | 7-(2-((2,7-dicyclopropyl-1,2,3,4- tetrahydroisoquinolin-6-yl)amino)- 5-(trifluoromethyl)pyrimidin-4-yl)- 4-(oxetan-3-yl)-3,4- dihydrothieno[2,3-f][1,4]thiazepin- 5(2H)-one 1,1-dioxide |
| 302 | 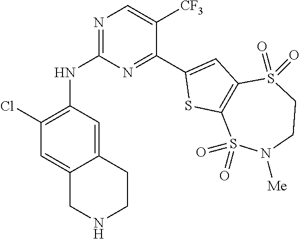 [SMILES: CN1CCS(=O)(=O)c2cc(-c3nc(Nc4cc5c(cc4Cl)CNCC5)ncc3C(F)(F)F)sc2S1(=O)=O] | 7-(2-((7-chloro-1,2,3,4- tetrahydroisoquinolin-6-yl)amino)- 5-(trifluoromethyl)pyrimidin-4-yl)- 2-methyl-3,4-dihydro-2H- thieno[3,2-f][1,5,2]dithiazepine 1,1,5,5-tetraoxide |
| 303 | 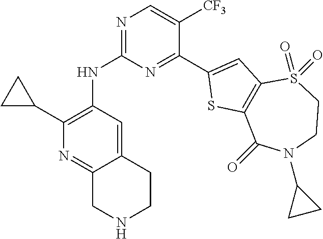 [SMILES: O=C1c2sc(-c3nc(Nc4cc5c(nc4C4CC4)CNCC5)ncc3C(F)(F)F)cc2S(=O)(=O)CCN1C1CC1] | 4-cyclopropyl-7-(2-((2- cyclopropyl-5,6,7,8-tetrahydro-1,7- naphthyridin-3-yl)amino)-5- (trifluoromethyl)pyrimidin-4-yl)- 3,4-dihydrothieno[2,3- f][1,4]thiazepin-5(2H)-one 1,1- dioxide |
| 304 | 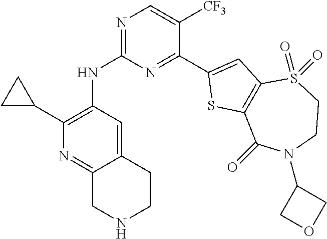 [SMILES: O=C1c2sc(-c3nc(Nc4cc5c(nc4C4CC4)CNCC5)ncc3C(F)(F)F)cc2S(=O)(=O)CCN1C1COC1] | 7-(2-((2-cyclopropyl-5,6,7,8- tetrahydro-1,7-naphthyridin-3- yl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 305 | 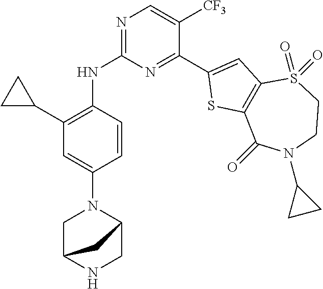 [SMILES: O=C1c2sc(-c3nc(Nc4ccc(N5C[C@@H]6C[C@H]5CN6)cc4C4CC4)ncc3C(F)(F)F)cc2S(=O)(=O)CCN1C1CC1] | 7-(2-((4-((1S,4S)-2,5- diazabicyclo[2.2.1]heptan-2-yl)-2- cyclopropylphenyl)amino)-5- (trifluoromethyl)pyrimidin-4-yl)-4- cyclopropyl-3,4-dihydrothieno[2,3- f][1,4]thiazepin-5(2H)-one 1,1- dioxide |
| 306 | 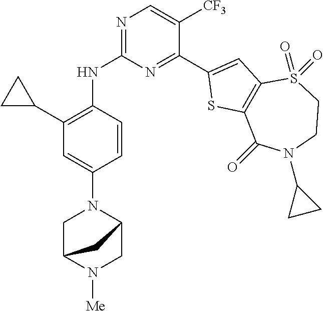 [SMILES: CN1C[C@@H]2C[C@H]1CN2c1ccc(Nc2ncc(C(F)(F)F)c(-c3cc4c(s3)C(=O)N(C3CC3)CCS4(=O)=O)n2)c(C2CC2)c1] | 4-Cyclopropyl-7-(2-((2- cyclopropyl-4-((1S,4S)-5-methyl- 2,5-diazabicyclo[2.2.1]heptan-2- yl)phenyl)amino)-5- (trifluoromethyl)pyrimidin-4-yl)- 3,4-dihydrothieno[2,3- f][1,4]thiazepin-5(2H)-one 1,1- dioxide |
| 307 | 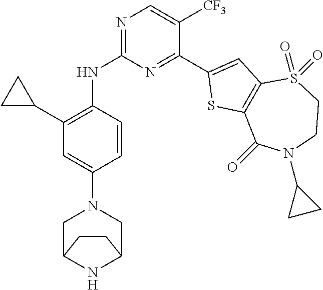 [SMILES: O=C1c2sc(-c3nc(Nc4ccc(N5CC6CCC(C5)N6)cc4C4CC4)ncc3C(F)(F)F)cc2S(=O)(=O)CCN1C1CC1] | 7-(2-((4-((1R,5S)-3,8- diazabicyclo[3.2.1]octan-3-yl)-2- cyclopropylphenyl)amino)-5- (trifluoromethyl)pyrimidin-4-yl)-4- cyclopropyl-3,4-dihydrothieno[2,3- f][1,4]thiazepin-5(2H)-one 1,1- dioxide |
| 308 | 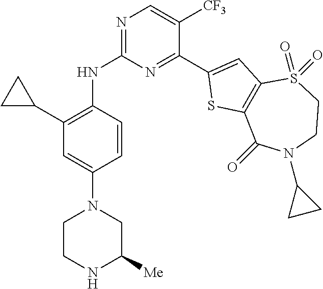 [SMILES: C[C@@H]1CN(c2ccc(Nc3ncc(C(F)(F)F)c(-c4cc5c(s4)C(=O)N(C4CC4)CCS5(=O)=O)n3)c(C3CC3)c2)CCN1] | (R)-4-cyclopropyl-7-(2-((2- cyclopropyl-4-(3-methylpiperazin- 1-yl)phenyl)amino)-5- (trifluoromethyl)pyrimidin-4-yl)- 3,4-dihydrothieno[2,3- f][1,4]thiazepin-5(2H)-one 1,1- dioxide |
| 309 | 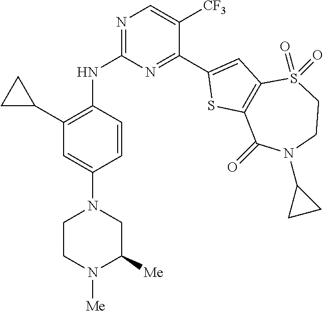 [SMILES: C[C@@H]1CN(c2ccc(Nc3ncc(C(F)(F)F)c(-c4cc5c(s4)C(=O)N(C4CC4)CCS5(=O)=O)n3)c(C3CC3)c2)CCN1C] | (R)-4-cyclopropyl-7-(2-((2- cyclopropyl-4-(3,4- dimethylpiperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 310 | 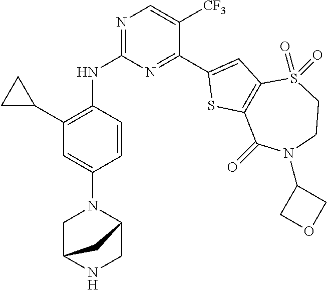 [SMILES: O=C1c2sc(-c3nc(Nc4ccc(N5C[C@@H]6C[C@H]5CN6)cc4C4CC4)ncc3C(F)(F)F)cc2S(=O)(=O)CCN1C1COC1] | 7-(2-((4-((1S,4S)-2,5- diazabicyclo[2.2.1]heptan-2-yl)-2- cyclopropylphenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 311 | 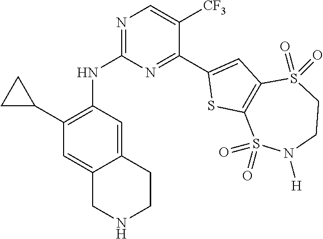 [SMILES: O=S1(=O)CCNS(=O)(=O)c2sc(-c3nc(Nc4cc5c(cc4C4CC4)CNCC5)ncc3C(F)(F)F)cc21] | 7-(2-((7-cyclopropyl-1,2,3,4- tetrahydroisoquinolin-6-yl)amino)- 5-(trifluoromethyl)pyrimidin-4-yl)- 3,4-dihydro-2H-thieno[3,2- f][1,5,2]dithiazepine 1,1,5,5- tetraoxide |
| 312 | 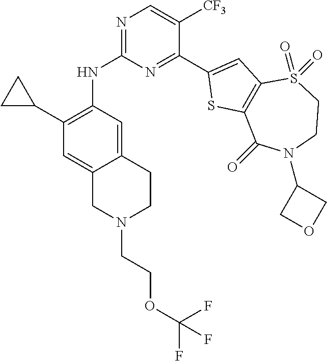 [SMILES: O=C1c2sc(-c3nc(Nc4cc5c(cc4C4CC4)CN(CCOC(F)(F)F)CC5)ncc3C(F)(F)F)cc2S(=O)(=O)CCN1C1COC1] | 7-(2-((7-cyclopropyl-2-(2- (trifluoromethoxy)ethyl)-1,2,3,4- tetrahydroisoquinolin-6-yl)amino)- 5-(trifluoromethyl)pyrimidin-4-yl)- 4-(oxetan-3-yl)-3,4- dihydrothieno[2,3-f] [1,4]thiazepin- 5(2H)-one 1,1-dioxide |
| 313 | 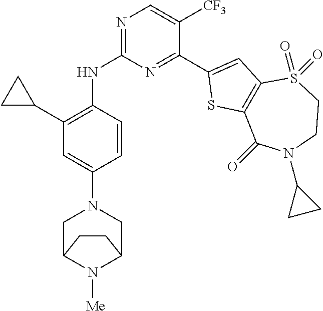 [SMILES: CN1C2CCC1CN(c1ccc(Nc3ncc(C(F)(F)F)c(-c4cc5c(s4)C(=O)N(C4CC4)CCS5(=O)=O)n3)c(C3CC3)c1)C2] | 4-cyclopropyl-7-(2-((2- cyclopropyl-4-((1R,5S)-8-methyl- 3,8-diazabicyclo[3.2.1]octan-3- yl)phenyl)amino)-5- (trifluoromethyl)pyrimidin-4-yl)- 3,4-dihydrothieno[2,3- f][1,4]thiazepin-5(2H)-one 1,1- dioxide |
| 314 | 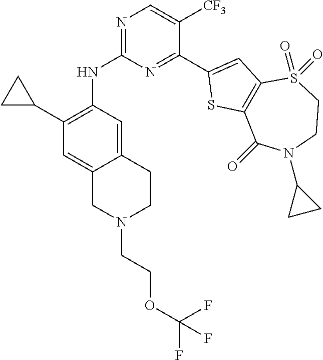 [SMILES: O=C1c2sc(-c3nc(Nc4cc5c(cc4C4CC4)CN(CCOC(F)(F)F)CC5)ncc3C(F)(F)F)cc2S(=O)(=O)CCN1C1CC1] | 4-cyclopropyl-7-(2-((7- cyclopropyl-2-(2- (trifluoromethoxy)ethyl)-1,2,3,4- tetrahydroisoquinolin-6-yl)amino)- 5-(trifluoromethyl)pyrimidin-4-yl)- 3,4-dihydrothieno[2,3- f][1,4]thiazepin-5(2H)-one 1,1- dioxide |
| 315 | 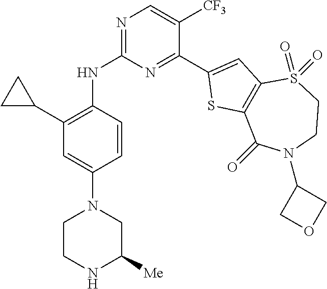 [SMILES: C[C@@H]1CN(c2ccc(Nc3ncc(C(F)(F)F)c(-c4cc5c(s4)C(=O)N(C4COC4)CCS5(=O)=O)n3)c(C3CC3)c2)CCN1] | (R)-7-(2-((2-cyclopropyl-4-(3- methylpiperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 316 | 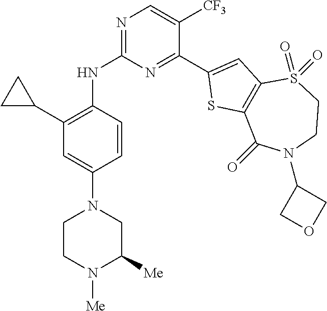 [SMILES: C[C@@H]1CN(c2ccc(Nc3ncc(C(F)(F)F)c(-c4cc5c(s4)C(=O)N(C4COC4)CCS5(=O)=O)n3)c(C3CC3)c2)CCN1C] | (R)-7-(2-((2-cyclopropyl-4-(3,4- dimethylpiperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 317 | 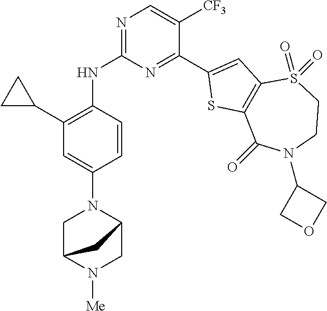 [SMILES: CN1C[C@@H]2C[C@H]1CN2c1ccc(Nc2ncc(C(F)(F)F)c(-c3cc4c(s3)C(=O)N(C3COC3)CCS4(=O)=O)n2)c(C2CC2)c1] | 7-(2-((2-cyclopropyl-4-((1S,4S)-5- methyl-2,5- diazabicyclo[2.2.1]heptan-2- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 318 | 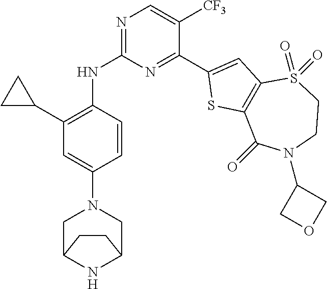 [SMILES: O=C1c2sc(-c3nc(Nc4ccc(N5CC6CCC(C5)N6)cc4C4CC4)ncc3C(F)(F)F)cc2S(=O)(=O)CCN1C1COC1] | 7-(2-((4-((1R,5S)-3,8- diazabicyclo[3.2.1]octan-3-yl)-2- cyclopropylphenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 319 | 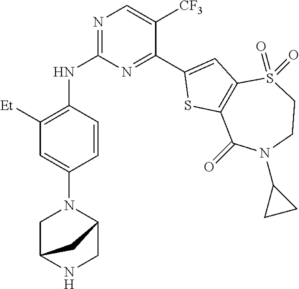 [SMILES: CCc1cc(N2C[C@@H]3C[C@H]2CN3)ccc1Nc1ncc(C(F)(F)F)c(-c2cc3c(s2)C(=O)N(C2CC2)CCS3(=O)=O)n1] | 7-(2-((4-((1S,4S)-2,5- diazabicyclo[2.2.1]heptan-2-yl)-2- ethylphenyl)amino)-5- (trifluoromethyl)pyrimidin-4-yl)-4- cyclopropyl-3,4-dihydrothieno[2,3- f][1,4]thiazepin-5(2H)-one 1,1- dioxide |
| 320 | 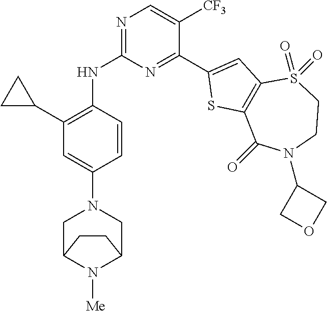 [SMILES: CN1C2CCC1CN(c1ccc(Nc3ncc(C(F)(F)F)c(-c4cc5c(s4)C(=O)N(C4COC4)CCS5(=O)=O)n3)c(C3CC3)c1)C2] | 7-(2-((2-cyclopropyl-4-((1R,5S)-8- methyl-3,8- diazabicyclo[3.2.1]octan-3- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 321 | 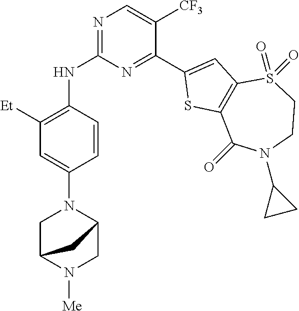 [SMILES: CCc1cc(N2C[C@@H]3C[C@H]2CN3C)ccc1Nc1ncc(C(F)(F)F)c(-c2cc3c(s2)C(=O)N(C2CC2)CCS3(=O)=O)n1] | 4-cyclopropyl-7-(2-((2-ethyl-4- ((1S,4S)-5-methyl-2,5- diazabicyclo[2.2.1]heptan-2- yl)phenyl)amino)-5- (trifluoromethyl)pyrimidin-4-yl)- 3,4-dihydrothieno[2,3- f][1,4]thiazepin-5(2H)-one 1,1- dioxide |
| 322 | 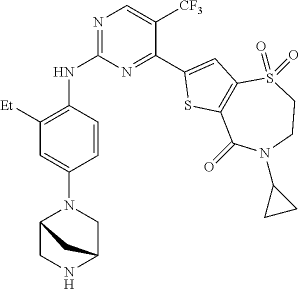 [SMILES: CCc1cc(N2C[C@H]3C[C@@H]2CN3)ccc1Nc1ncc(C(F)(F)F)c(-c2cc3c(s2)C(=O)N(C2CC2)CCS3(=O)=O)n1] | 7-(2-((4-((1R,4R)-2,5- diazabicyclo[2.2.1]heptan-2-yl)-2- ethylphenyl)amino)-5- (trifluoromethyl)pyrimidin-4-yl)-4- cyclopropyl-3,4-dihydrothieno[2,3- f][1,4]thiazepin-5(2H)-one 1,1- dioxide |
| 323 | 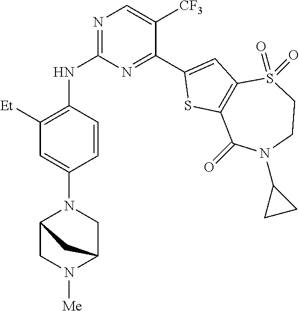 [SMILES: CCc1cc(N2C[C@H]3C[C@@H]2CN3C)ccc1Nc1ncc(C(F)(F)F)c(-c2cc3c(s2)C(=O)N(C2CC2)CCS3(=O)=O)n1] | 4-cyclopropyl-7-(2-((2-ethyl-4- ((1R,4R)-5-methyl-2,5- diazabicyclo[2.2.1]heptan-2- yl)phenyl)amino)-5- (trifluoromethyl)pyrimidin-4-yl)- 3,4-dihydrothieno[2,3- f][1,4]thiazepin-5(2H)-one 1,1- dioxide |
| 324 | 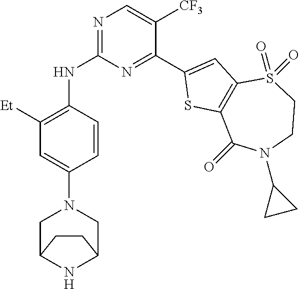 [SMILES: CCc1cc(N2CC3CCC(C2)N3)ccc1Nc1ncc(C(F)(F)F)c(-c2cc3c(s2)C(=O)N(C2CC2)CCS3(=O)=O)n1] | 7-(2-((4-((1R,5S)-3,8- diazabicyclo[3.2.1]octan-3-yl)-2- ethylphenyl)amino)-5- (trifluoromethyl)pyrimidin-4-yl)-4- cyclopropyl-3,4-dihydrothieno[2,3- f][1,4]thiazepin-5(2H)-one 1,1- dioxide |
| 325 | 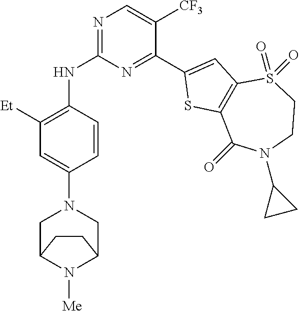 [SMILES: CCc1cc(N2CC3CCC(C2)N3C)ccc1Nc1ncc(C(F)(F)F)c(-c2cc3c(s2)C(=O)N(C2CC2)CCS3(=O)=O)n1] | 4-cyclopropyl-7-(2-((2-ethyl-4- ((1R,5S)-8-methyl-3,8- diazabicyclo[3.2.1]octan-3- yl)phenyl)amino)-5- (trifluoromethyl)pyrimidin-4-yl)- 3,4-dihydrothieno[2,3- f][1,4]thiazepin-5(2H)-one 1,1- dioxide |
| 326 | 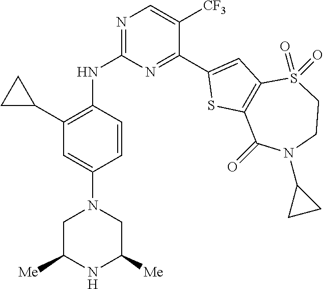 [SMILES: C[C@@H]1CN(c2ccc(Nc3ncc(C(F)(F)F)c(-c4cc5c(s4)C(=O)N(C4CC4)CCS5(=O)=O)n3)c(C3CC3)c2)C[C@H](C)N1] | 4-cyclopropyl-7-(2-((2- cyclopropyl-4-((3S,5R)-3,5- dimethylpiperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 327 | 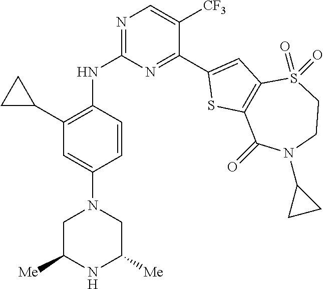 [SMILES: C[C@H]1CN(c2ccc(Nc3ncc(C(F)(F)F)c(-c4cc5c(s4)C(=O)N(C4CC4)CCS5(=O)=O)n3)c(C3CC3)c2)C[C@H](C)N1] | 4-cyclopropyl-7-(2-((2- cyclopropyl-4-((3S,5S)-3,5- dimethylpiperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 328 | 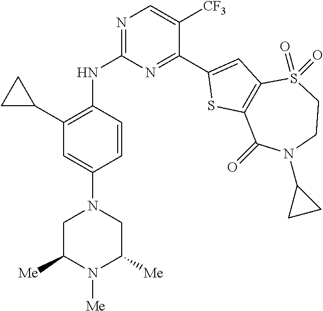 [SMILES: C[C@H]1CN(c2ccc(Nc3ncc(C(F)(F)F)c(-c4cc5c(s4)C(=O)N(C4CC4)CCS5(=O)=O)n3)c(C3CC3)c2)C[C@H](C)N1C] | 4-cyclopropyl-7-(2-((2- cyclopropyl-4-((3S,5S)-3,4,5- trimethylpiperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 329 | 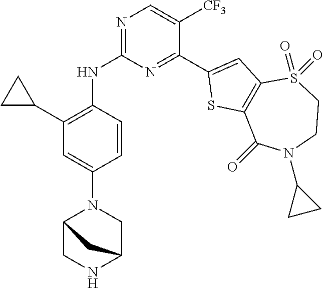 [SMILES: O=C1c2sc(-c3nc(Nc4ccc(N5C[C@H]6C[C@@H]5CN6)cc4C4CC4)ncc3C(F)(F)F)cc2S(=O)(=O)CCN1C1CC1] | 7-(2-((4-((1R,4R)-2,5- diazabicyclo[2.2.1]heptan-2-yl)-2- cyclopropylphenyl)amino)-5- (trifluoromethyl)pyrimidin-4-yl)-4- cyclopropyl-3,4-dihydrothieno[2,3- f][1,4]thiazepin-5(2H)-one 1,1- dioxide |
| 330 | 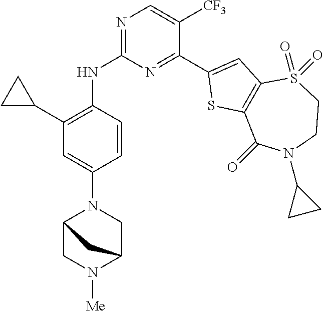 [SMILES: CN1C[C@H]2C[C@@H]1CN2c1ccc(Nc2ncc(C(F)(F)F)c(-c3cc4c(s3)C(=O)N(C3CC3)CCS4(=O)=O)n2)c(C2CC2)c1] | 4-cyclopropyl-7-(2-((2- cyclopropyl-4-((1R,4R)-5-methyl- 2,5-diazabicyclo[2.2.1]heptan-2- yl)phenyl)amino)-5- (trifluoromethyl)pyrimidin-4-yl)- 3,4-dihydrothieno[2,3- f][1,4]thiazepin-5(2H)-one 1,1- dioxide |
| 331 | 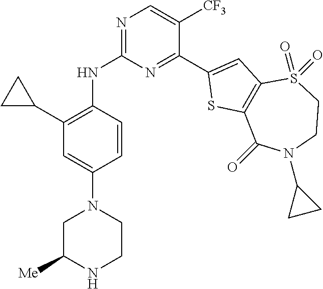 [SMILES: C[C@H]1CN(c2ccc(Nc3ncc(C(F)(F)F)c(-c4cc5c(s4)C(=O)N(C4CC4)CCS5(=O)=O)n3)c(C3CC3)c2)CCN1] | (S)-4-cyclopropyl-7-(2-((2- cyclopropyl-4-(3-methylpiperazin- 1-yl)phenyl)amino)-5- (trifluoromethyl)pyrimidin-4-yl)- 3,4-dihydrothieno[2,3- f][1,4]thiazepin-5(2H)-one 1,1- dioxide |
| 332 | 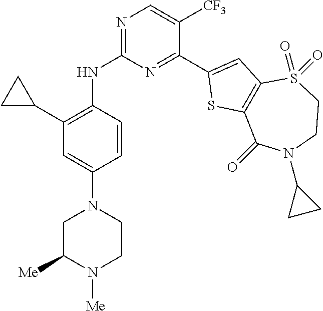 [SMILES: C[C@H]1CN(c2ccc(Nc3ncc(C(F)(F)F)c(-c4cc5c(s4)C(=O)N(C4CC4)CCS5(=O)=O)n3)c(C3CC3)c2)CCN1C] | (S)-4-cyclopropyl-7-(2-((2- cyclopropyl-4-(3,4- dimethylpiperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 333 | 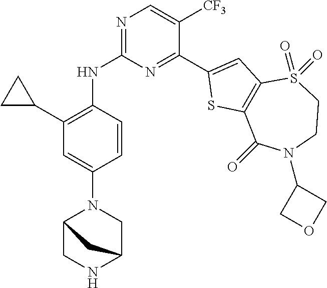 [SMILES: O=C1c2sc(-c3nc(Nc4ccc(N5C[C@H]6C[C@@H]5CN6)cc4C4CC4)ncc3C(F)(F)F)cc2S(=O)(=O)CCN1C1COC1] | 7-(2-((4-((1R,4R)-2,5- diazabicyclo[2.2.1]heptan-2-yl)-2- cyclopropylphenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 334 | 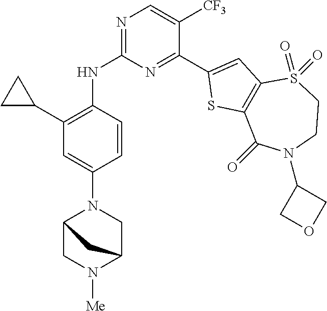 [SMILES: CN1C[C@H]2C[C@@H]1CN2c1ccc(Nc2ncc(C(F)(F)F)c(-c3cc4c(s3)C(=O)N(C3COC3)CCS4(=O)=O)n2)c(C2CC2)c1] | 7-(2-((2-cyclopropyl-4-((1R,4R)-5- methyl-2,5- diazabicyclo[2.2.1]heptan-2- yl)phenyl)amino)-5- (trifluoromethyl)pyrimidin-4-yl)-4- (oxetan-3-y1)-3,4- dihydrothieno[2,3-f][1,4]thiazepin- 5(2H)-one 1,1-dioxide |
| 335 | 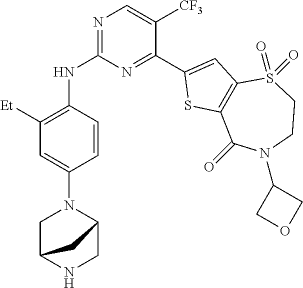 [SMILES: CCc1cc(N2C[C@@H]3C[C@H]2CN3)ccc1Nc1ncc(C(F)(F)F)c(-c2cc3c(s2)C(=O)N(C2COC2)CCS3(=O)=O)n1] | 7-(2-((4-((1S,4S)-2,5- diazabicyclo[2.2.1]heptan-2-yl)-2- ethylphenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 336 | 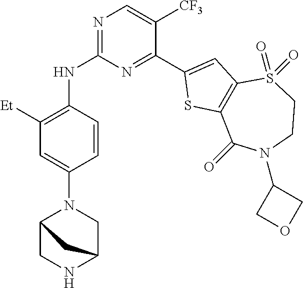 [SMILES: CCc1cc(N2C[C@H]3C[C@@H]2CN3)ccc1Nc1ncc(C(F)(F)F)c(-c2cc3c(s2)C(=O)N(C2COC2)CCS3(=O)=O)n1] | 7-(2-((4-((1R,4R)-2,5- diazabicyclo[2.2.1]heptan-2-yl)-2- ethylphenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 337 | 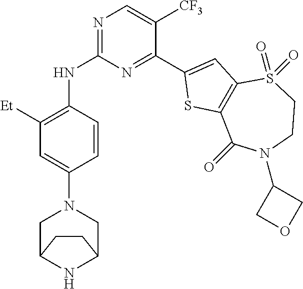 [SMILES: CCc1cc(N2CC3CCC(C2)N3)ccc1Nc1ncc(C(F)(F)F)c(-c2cc3c(s2)C(=O)N(C2COC2)CCS3(=O)=O)n1] | 7-(2-((4-((1R,5S)-3,8- diazabicyclo[3.2.1]octan-3-yl)-2- ethylphenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 338 | 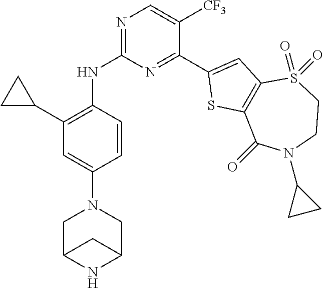 [SMILES: O=C1c2sc(-c3nc(Nc4ccc(N5CC6CC(C5)N6)cc4C4CC4)ncc3C(F)(F)F)cc2S(=O)(=O)CCN1C1CC1] | 7-(2-((4-(3,6- diazabicyclo[3.1.1]heptan-3-yl)-2- cyclopropylphenyl)amino)-5- (trifluoromethyl)pyrimidin-4-yl)-4- cyclopropyl-3,4-dihydrothieno[2,3- f][1,4]thiazepin-5(2H)-one 1,1- dioxide |
| 339 | 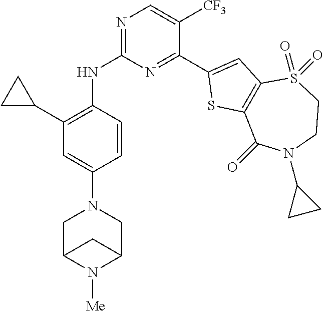 [SMILES: CN1C2CC1CN(c1ccc(Nc3ncc(C(F)(F)F)c(-c4cc5c(s4)C(=O)N(C4CC4)CCS5(=O)=O)n3)c(C3CC3)c1)C2] | 4-cyclopropyl-7-(2-((2- cyclopropyl-4-(6-methyl-3,6- diazabicyclo[3.1.1]heptan-3- yl)phenyl)amino)-5- (trifluoromethyl)pyrimidin-4-yl)- 3,4-dihydrothieno[2,3- f][1,4]thiazepin-5(2H)-one 1,1- dioxide |
| 340 | 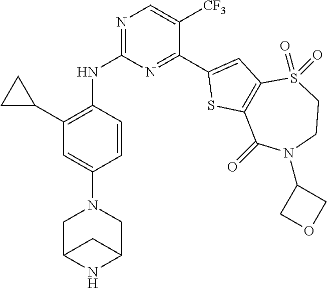 [SMILES: O=C1c2sc(-c3nc(Nc4ccc(N5CC6CC(C5)N6)cc4C4CC4)ncc3C(F)(F)F)cc2S(=O)(=O)CCN1C1COC1] | 7-(2-((4-(3,6- diazabicyclo[3.1.1]heptan-3-yl)-2- cyclopropylphenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 341 | 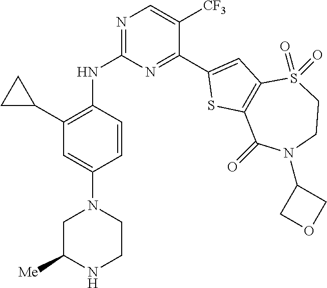 [SMILES: C[C@H]1CN(c2ccc(Nc3ncc(C(F)(F)F)c(-c4cc5c(s4)C(=O)N(C4COC4)CCS5(=O)=O)n3)c(C3CC3)c2)CCN1] | (S)-7-(2-((2-Cyclopropyl-4-(3- methylpiperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 342 | 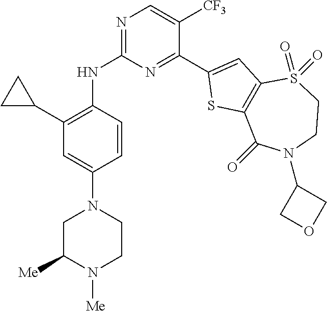 [SMILES: C[C@H]1CN(c2ccc(Nc3ncc(C(F)(F)F)c(-c4cc5c(s4)C(=O)N(C4COC4)CCS5(=O)=O)n3)c(C3CC3)c2)CCN1C] | (S)-7-(2-((2-cyclopropyl-4-(3,4- dimethylpiperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 343 | 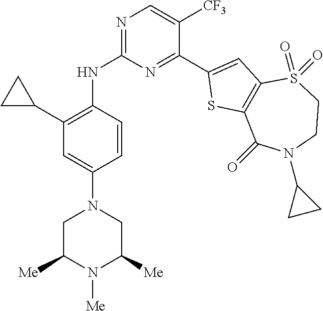 [SMILES: C[C@@H]1CN(c2ccc(Nc3ncc(C(F)(F)F)c(-c4cc5c(s4)C(=O)N(C4CC4)CCS5(=O)=O)n3)c(C3CC3)c2)C[C@H](C)N1C] | 4-Cyclopropyl-7-(2-((2- cyclopropyl-4-((3S,5R)-3,4,5- trimethylpiperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 344 | 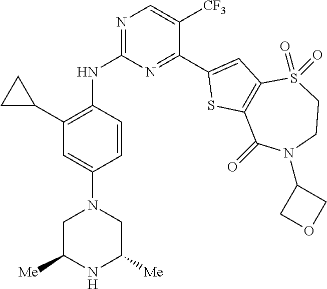 [SMILES: C[C@H]1CN(c2ccc(Nc3ncc(C(F)(F)F)c(-c4cc5c(s4)C(=O)N(C4COC4)CCS5(=O)=O)n3)c(C3CC3)c2)C[C@H](C)N1] | 7-(2-((2-Cyclopropyl-4-((3S,5S)- 3,5-dimethylpiperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 345 | 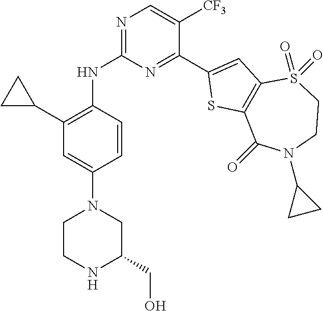 [SMILES: O=C1c2sc(-c3nc(Nc4ccc(N5CCN[C@@H](CO)C5)cc4C4CC4)ncc3C(F)(F)F)cc2S(=O)(=O)CCN1C1CC1] | (R)-4-cyclopropyl-7-(2-((2- cyclopropyl-4-(3- (hydroxymethyl)piperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 346 | 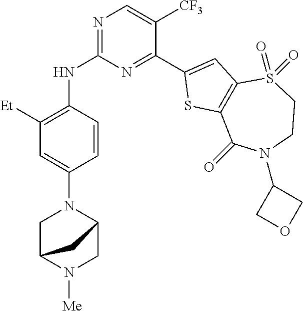 [SMILES: CCc1cc(N2C[C@@H]3C[C@H]2CN3C)ccc1Nc1ncc(C(F)(F)F)c(-c2cc3c(s2)C(=O)N(C2COC2)CCS3(=O)=O)n1] | 7-(2-((2-Ethyl-4-((1S,4S)-5- methyl-2,5- diazabicyclo[2.2.1]heptan-2- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 347 | 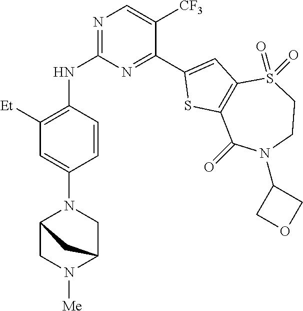 [SMILES: CCc1cc(N2C[C@H]3C[C@@H]2CN3C)ccc1Nc1ncc(C(F)(F)F)c(-c2cc3c(s2)C(=O)N(C2COC2)CCS3(=O)=O)n1] | 7-(2-((2-ethyl-4-((1R,4R)-5- methyl-2,5- diazabicyclo[2.2.1]heptan-2- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 348 | 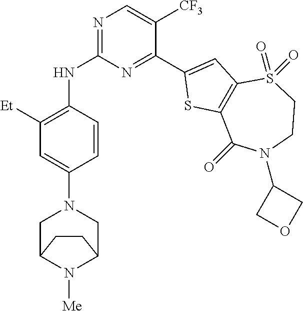 [SMILES: CCc1cc(N2CC3CCC(C2)N3C)ccc1Nc1ncc(C(F)(F)F)c(-c2cc3c(s2)C(=O)N(C2COC2)CCS3(=O)=O)n1] | 7-(2-((2-ethyl-4-((1R,5S)-8-methyl- 3,8-diazabicyclo[3.2.1]octan-3- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 349 | 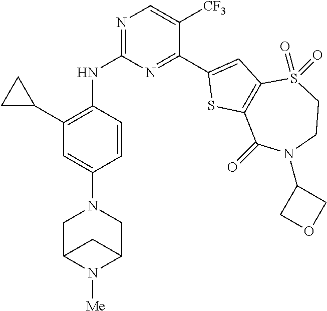 [SMILES: CN1C2CC1CN(c1ccc(Nc3ncc(C(F)(F)F)c(-c4cc5c(s4)C(=O)N(C4COC4)CCS5(=O)=O)n3)c(C3CC3)c1)C2] | 7-(2-((2-cyclopropyl-4-(6-methyl- 3,6-diazabicyclo[3.1.1]heptan-3- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 350 | 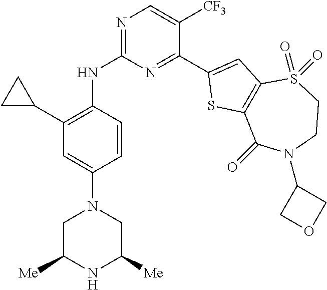 [SMILES: C[C@@H]1CN(c2ccc(Nc3ncc(C(F)(F)F)c(-c4cc5c(s4)C(=O)N(C4COC4)CCS5(=O)=O)n3)c(C3CC3)c2)C[C@H](C)N1] | 7-(2-((2-cyclopropyl-4-((3S,5R)- 3,5-dimethylpiperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 351 | 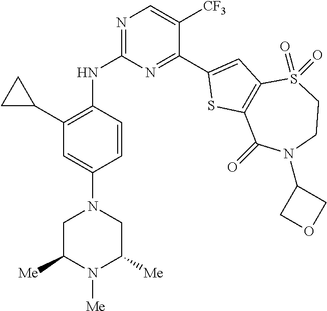 [SMILES: C[C@H]1CN(c2ccc(Nc3ncc(C(F)(F)F)c(-c4cc5c(s4)C(=O)N(C4COC4)CCS5(=O)=O)n3)c(C3CC3)c2)C[C@H](C)N1C] | 7-(2-((2-cyclopropyl-4-((3S,5S)- 3,4,5-trimethylpiperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 352 | 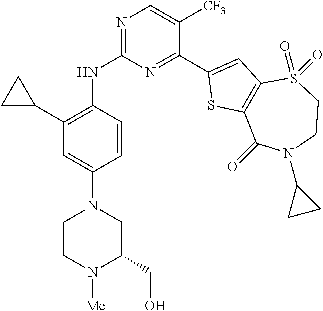 [SMILES: CN1CCN(c2ccc(Nc3ncc(C(F)(F)F)c(-c4cc5c(s4)C(=O)N(C4CC4)CCS5(=O)=O)n3)c(C3CC3)c2)C[C@@H]1CO] | (R)-4-cyclopropyl-7-(2-((2- cyclopropyl-4-(3-(hydroxymethyl)- 4-methylpiperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 353 | 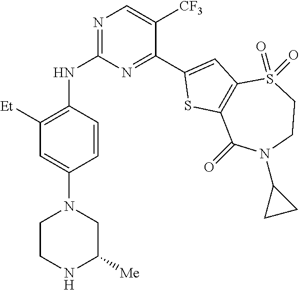 [SMILES: CCc1cc(N2CCN[C@@H](C)C2)ccc1Nc1ncc(C(F)(F)F)c(-c2cc3c(s2)C(=O)N(C2CC2)CCS3(=O)=O)n1] | (S)-4-cyclopropyl-7-(2-((2-ethyl-4- (3-methylpiperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 354 | 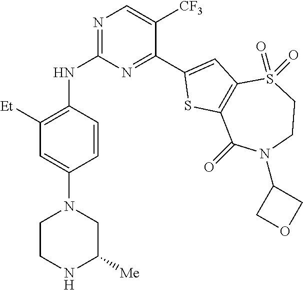 [SMILES: CCc1cc(N2CCN[C@@H](C)C2)ccc1Nc1ncc(C(F)(F)F)c(-c2cc3c(s2)C(=O)N(C2COC2)CCS3(=O)=O)n1] | (S)-7-(2-((2-ethyl-4-(3- methylpiperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 355 | 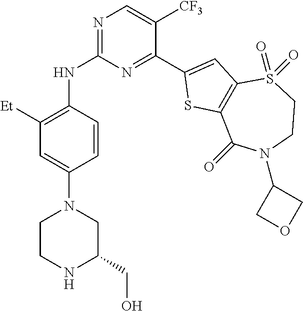 [SMILES: CCc1cc(N2CCN[C@@H](CO)C2)ccc1Nc1ncc(C(F)(F)F)c(-c2cc3c(s2)C(=O)N(C2COC2)CCS3(=O)=O)n1] | (R)-7-(2-((2-ethyl-4-(3- (hydroxymethyl)piperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 356 | 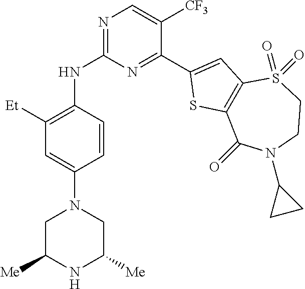 [SMILES: CCc1cc(N2C[C@H](C)N[C@@H](C)C2)ccc1Nc1ncc(C(F)(F)F)c(-c2cc3c(s2)C(=O)N(C2CC2)CCS3(=O)=O)n1] | 4-cyclopropyl-7-(2-((4-((3S,5S)- 3,5-dimethylpiperazin-1-yl)-2- ethylphenyl)amino)-5- (trifluoromethyl)pyrimidin-4-yl)- 3,4-dihydrothieno[2,3- f][1,4]thiazepin-5(2H)-one 1,1- dioxide |
| 357 | 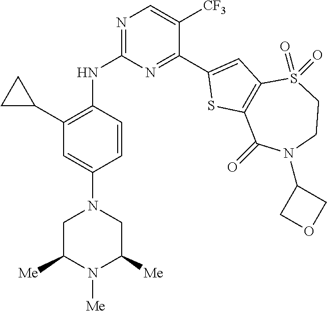 [SMILES: C[C@@H]1CN(c2ccc(Nc3ncc(C(F)(F)F)c(-c4cc5c(s4)C(=O)N(C4COC4)CCS5(=O)=O)n3)c(C3CC3)c2)C[C@H](C)N1C] | 7-(2-((2-cyclopropyl-4-((3S,5R)- 3,4,5-trimethylpiperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 358 | 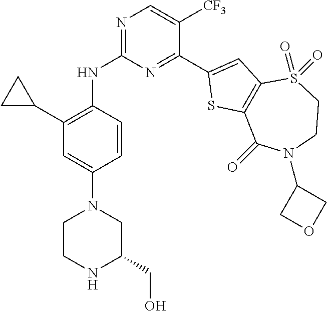 [SMILES: O=C1c2sc(-c3nc(Nc4ccc(N5CCN[C@@H](CO)C5)cc4C4CC4)ncc3C(F)(F)F)cc2S(=O)(=O)CCN1C1COC1] | (R)-7-(2-((2-cyclopropyl-4-(3- (hydroxymethyl)piperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 359 | 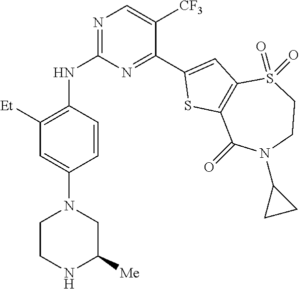 [SMILES: CCc1cc(N2CCN[C@H](C)C2)ccc1Nc1ncc(C(F)(F)F)c(-c2cc3c(s2)C(=O)N(C2CC2)CCS3(=O)=O)n1] | (R)-4-cyclopropyl-7-(2-((2-ethyl-4- (3-methylpiperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 360 | 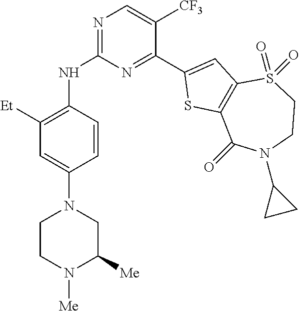 [SMILES: CCc1cc(N2CCN(C)[C@H](C)C2)ccc1Nc1ncc(C(F)(F)F)c(-c2cc3c(s2)C(=O)N(C2CC2)CCS3(=O)=O)n1] | (R)-4-cyclopropyl-7-(2-((4-(3,4- dimethylpiperazin-1-yl)-2- ethylphenyl)amino)-5- (trifluoromethyl)pyrimidin-4-yl)- 3,4-dihydrothieno[2,3- f][1,4]thiazepin-5(2H)-one 1,1- dioxide |
| 361 | 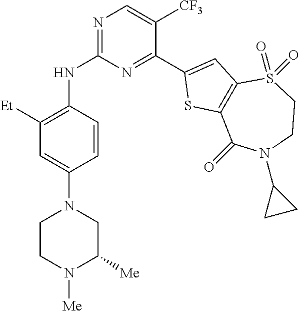 [SMILES: CCc1cc(N2CCN(C)[C@@H](C)C2)ccc1Nc1ncc(C(F)(F)F)c(-c2cc3c(s2)C(=O)N(C2CC2)CCS3(=O)=O)n1] | (S)-4-cyclopropyl-7-(2-((4-(3,4- dimethylpiperazin-1-yl)-2- ethylphenyl)amino)-5- (trifluoromethyl)pyrimidin-4-yl)- 3,4-dihydrothieno[2,3- f][1,4]thiazepin-5(2H)-one 1,1- dioxide |
| 362 | 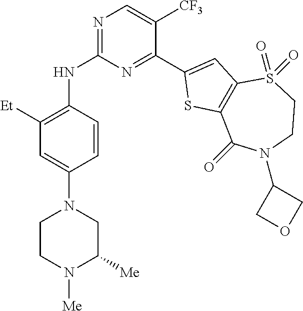 [SMILES: CCc1cc(N2CCN(C)[C@@H](C)C2)ccc1Nc1ncc(C(F)(F)F)c(-c2cc3c(s2)C(=O)N(C2COC2)CCS3(=O)=O)n1] | (S)-7-(2-((4-(3,4- dimethylpiperazin-1-yl)-2- ethylphenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 363 | 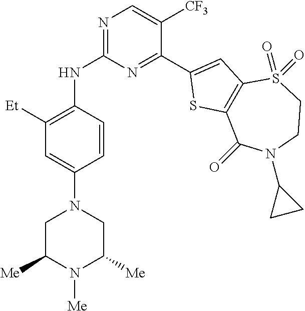 [SMILES: CCc1cc(N2C[C@H](C)N(C)[C@@H](C)C2)ccc1Nc1ncc(C(F)(F)F)c(-c2cc3c(s2)C(=O)N(C2CC2)CCS3(=O)=O)n1] | 4-cyclopropyl-7-(2-((2-ethyl-4- ((3S,5S)-3,4,5-trimethylpiperazin- 1-yl)phenyl)amino)-5- (trifluoromethyl)pyrimidin-4-yl)- 3,4-dihydrothieno[2,3- f][1,4]thiazepin-5(2H)-one 1,1- dioxide |
| 364 | 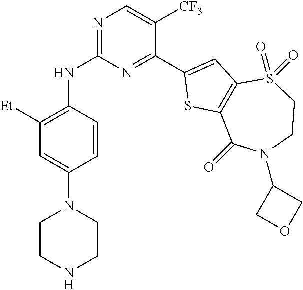 [SMILES: CCc1cc(N2CCNCC2)ccc1Nc1ncc(C(F)(F)F)c(-c2cc3c(s2)C(=O)N(C2COC2)CCS3(=O)=O)n1] | 7-(2-((2-ethyl-4-(piperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 365 | 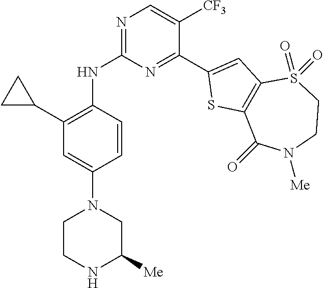 [SMILES: C[C@@H]1CN(c2ccc(Nc3ncc(C(F)(F)F)c(-c4cc5c(s4)C(=O)N(C)CCS5(=O)=O)n3)c(C3CC3)c2)CCN1] | (R)-7-(2-((2-cyclopropyl-4-(3- methylpiperazin-1- yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 366 | 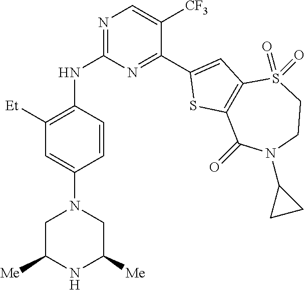 [SMILES: CCc1cc(N2C[C@@H](C)N[C@@H](C)C2)ccc1Nc1ncc(C(F)(F)F)c(-c2cc3c(s2)C(=O)N(C2CC2)CCS3(=O)=O)n1] | 4-cyclopropyl-7-(2-((4-((3S,5R)- 3,5-dimethylpiperazin-1-yl)-2- ethylphenyl)amino)-5- (trifluoromethyl)pyrimidin-4-yl)- 3,4-dihydrothieno[2,3- f][1,4]thiazepin-5(2H)-one 1,1- dioxide |
| 367 | 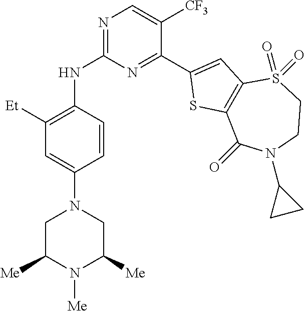 [SMILES: CCc1cc(N2C[C@@H](C)N(C)[C@@H](C)C2)ccc1Nc1ncc(C(F)(F)F)c(-c2cc3c(s2)C(=O)N(C2CC2)CCS3(=O)=O)n1] | 4-cyclopropyl-7-(2-((2-ethyl-4- ((3S,5R)-3,4,5-trimethylpiperazin- 1-yl)phenyl)amino)-5- (trifluoromethyl)pyrimidin-4-yl)- 3,4-dihydrothieno[2,3- f][1,4]thiazepin-5(2H)-one 1,1- dioxide |
| 368 | 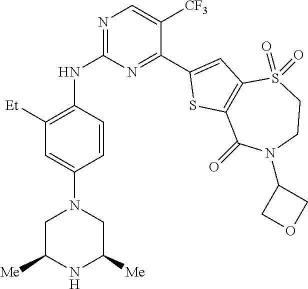 [SMILES: CCc1cc(N2C[C@@H](C)N[C@@H](C)C2)ccc1Nc1ncc(C(F)(F)F)c(-c2cc3c(s2)C(=O)N(C2COC2)CCS3(=O)=O)n1] | 7-(2-((4-((3S,5R)-3,5- dimethylpiperazin-1-yl)-2- ethylphenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 369 | 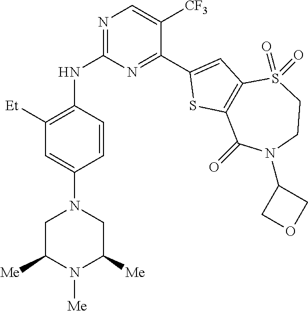 [SMILES: CCc1cc(N2C[C@@H](C)N(C)[C@@H](C)C2)ccc1Nc1ncc(C(F)(F)F)c(-c2cc3c(s2)C(=O)N(C2COC2)CCS3(=O)=O)n1] | 7-(2-((2-ethyl-4-((3S,5R)-3,4,5- trimethylpiperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 370 | 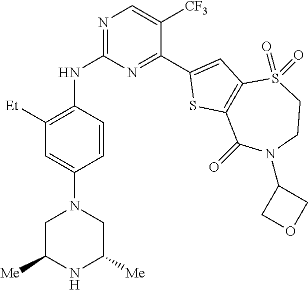 [SMILES: CCc1cc(N2C[C@H](C)N[C@@H](C)C2)ccc1Nc1ncc(C(F)(F)F)c(-c2cc3c(s2)C(=O)N(C2COC2)CCS3(=O)=O)n1] | 7-(2-((4-((3S,5S)-3,5- dimethylpiperazin-1-yl)-2- ethylphenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 371 | 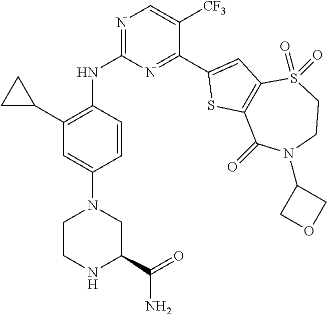 [SMILES: NC(=O)[C@@H]1CN(c2ccc(Nc3ncc(C(F)(F)F)c(-c4cc5c(s4)C(=O)N(C4COC4)CCS5(=O)=O)n3)c(C3CC3)c2)CCN1] | (S)-4-(3-cyclopropyl-4-((4-(4- (oxetan-3-yl)-1,1-dioxido-5-oxo- 2,3,4,5-tetrahydrothieno[2,3- f][1,4]thiazepin-7-yl)-5- (trifluoromethyl)pyrimidin-2- yl)amino)phenyl)piperazine-2- carboxamide |
| 372 | 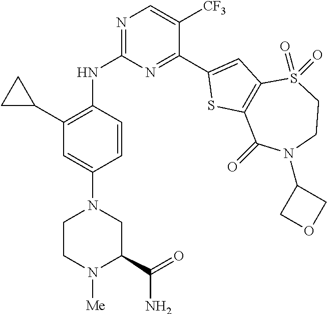 [SMILES: CN1CCN(c2ccc(Nc3ncc(C(F)(F)F)c(-c4cc5c(s4)C(=O)N(C4COC4)CCS5(=O)=O)n3)c(C3CC3)c2)C[C@H]1C(N)=O] | (S)-4-(3-Cyclopropyl-4-((4-(4- (oxetan-3-yl)-1,1-dioxido-5-oxo- 2,3,4,5-tetrahydrothieno[2,3- f][1,4]thiazepin-7-yl)-5- (trifluoromethyl)pyrimidin-2- yl)amino)phenyl)-1- methylpiperazine-2-carboxamide |
| 373 | 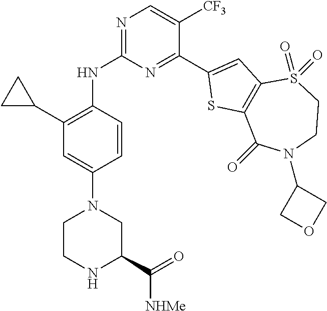 [SMILES: CNC(=O)[C@@H]1CN(c2ccc(Nc3ncc(C(F)(F)F)c(-c4cc5c(s4)C(=O)N(C4COC4)CCS5(=O)=O)n3)c(C3CC3)c2)CCN1] | (S)-4-(3-cyclopropyl-4-((4-(4- (oxetan-3-yl)-1,1-dioxido-5-oxo- 2,3,4,5-tetrahydrothieno[2,3- f][1,4]thiazepin-7-yl)-5- (trifluoromethyl)pyrimidin-2- yl)amino)phenyl)-N- methylpiperazine-2-carboxamide |
| 374 | 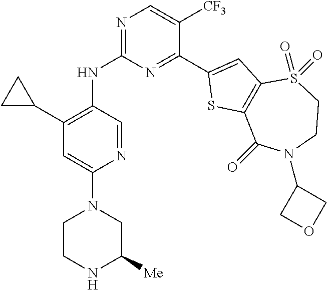 [SMILES: C[C@@H]1CN(c2cc(C3CC3)c(Nc3ncc(C(F)(F)F)c(-c4cc5c(s4)C(=O)N(C4COC4)CCS5(=O)=O)n3)cn2)CCN1] | (R)-7-(2-((4-cyclopropyl-6-(3- methylpiperazin-1-yl)pyridin-3- yl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 375 | 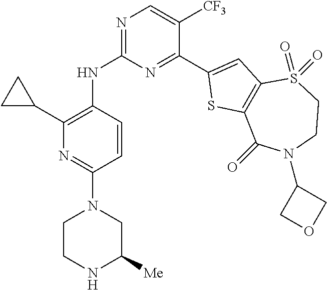 [SMILES: C[C@@H]1CN(c2ccc(Nc3ncc(C(F)(F)F)c(-c4cc5c(s4)C(=O)N(C4COC4)CCS5(=O)=O)n3)c(C3CC3)n2)CCN1] | (R)-7-(2-((2-cyclopropyl-6-(3- methylpiperazin-1-yl)pyridin-3- yl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 376 | 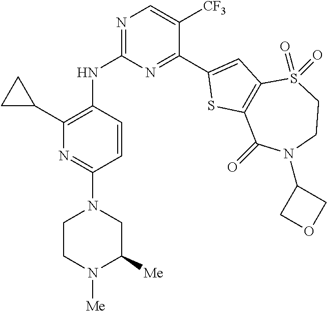 [SMILES: C[C@@H]1CN(c2ccc(Nc3ncc(C(F)(F)F)c(-c4cc5c(s4)C(=O)N(C4COC4)CCS5(=O)=O)n3)c(C3CC3)n2)CCN1C] | (R)-7-(2-((2-cyclopropyl-6-(3,4- dimethylpiperazin-1-yl)pyridin-3- yl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 377 | 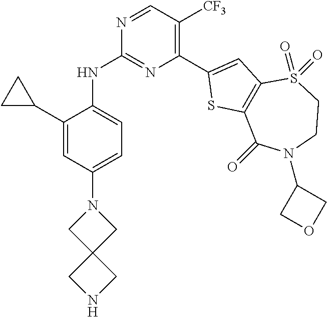 [SMILES: O=C1c2sc(-c3nc(Nc4ccc(N5CC6(CNC6)C5)cc4C4CC4)ncc3C(F)(F)F)cc2S(=O)(=O)CCN1C1COC1] | 7-(2-((2-Cyclopropyl-4-(2,6- diazaspiro[3.3]heptan-2- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 378 | 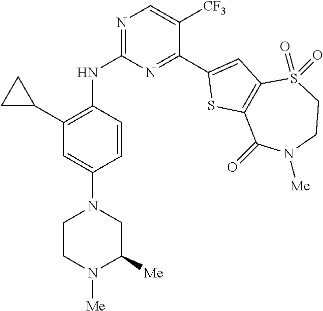 [SMILES: C[C@@H]1CN(c2ccc(Nc3ncc(C(F)(F)F)c(-c4cc5c(s4)C(=O)N(C)CCS5(=O)=O)n3)c(C3CC3)c2)CCN1C] | (R)-7-(2-((2-cyclopropyl-4-(3,4- dimethylpiperazin-1- yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 379 | 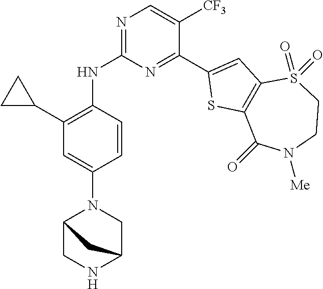 [SMILES: CN1CCS(=O)(=O)c2cc(-c3nc(Nc4ccc(N5C[C@H]6C[C@@H]5CN6)cc4C4CC4)ncc3C(F)(F)F)sc2C1=O] | 7-(2-((4-((1R,4R)-2,5- diazabicyclo[2.2.1]heptan-2-yl)-2- cyclopropylphenyl)amino)-5- (trifluoromethyl)pyrimidin-4-yl)-4- methyl-3,4-dihydrothieno[2,3- f][1,4]thiazepin-5(2H)-one 1,1- dioxide |
| 380 | 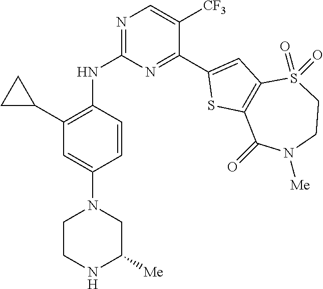 [SMILES: C[C@H]1CN(c2ccc(Nc3ncc(C(F)(F)F)c(-c4cc5c(s4)C(=O)N(C)CCS5(=O)=O)n3)c(C3CC3)c2)CCN1] | (S)-7-(2-((2-cyclopropyl-4-(3- methylpiperazin-1- yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 381 | 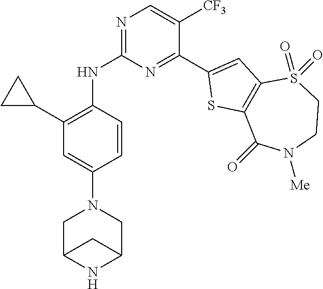 [SMILES: CN1CCS(=O)(=O)c2cc(-c3nc(Nc4ccc(N5CC6CC(C5)N6)cc4C4CC4)ncc3C(F)(F)F)sc2C1=O] | 7-(2-((4-(3,6- diazabicyclo[3.1.1]heptan-3-yl)-2- cyclopropylphenyl)amino)-5- (trifluoromethyl)pyrimidin-4-yl)-4- methyl-3,4-dihydrothieno[2,3- f][1,4]thiazepin-5(2H)-one 1,1- dioxide |
| 382 | 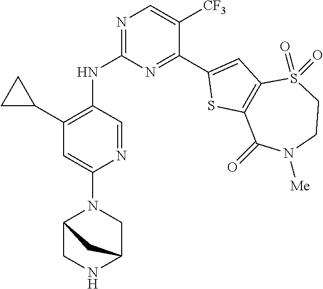 [SMILES: CN1CCS(=O)(=O)c2cc(-c3nc(Nc4cnc(N5C[C@H]6C[C@@H]5CN6)cc4C4CC4)ncc3C(F)(F)F)sc2C1=O] | 7-(2-((6-((1R,4R)-2,5- diazabicyclo[2.2.1]heptan-2-yl)-4- cyclopropylpyridin-3-yl)amino)-5- (trifluoromethyl)pyrimidin-4-yl)-4- methyl-3,4-dihydrothieno[2,3- f][1,4]thiazepin-5(2H)-one 1,1- dioxide |
| 383 | 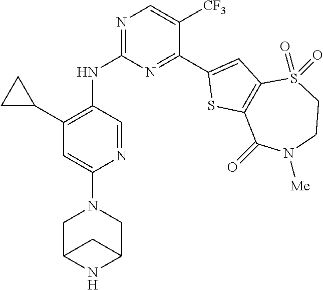 [SMILES: CN1CCS(=O)(=O)c2cc(-c3nc(Nc4cnc(N5CC6CC(C5)N6)cc4C4CC4)ncc3C(F)(F)F)sc2C1=O] | 7-(2-((6-(3,6- diazabicyclo[3.1.1]heptan-3-yl)-4- cyclopropylpyridin-3-yl)amino)-5- (trifluoromethyl)pyrimidin-4-yl)-4- methyl-3,4-dihydrothieno[2,3- f][1,4]thiazepin-5(2H)-one 1,1- dioxide |
| 384 | 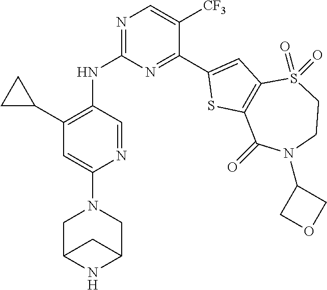 [SMILES: O=C1c2sc(-c3nc(Nc4cnc(N5CC6CC(C5)N6)cc4C4CC4)ncc3C(F)(F)F)cc2S(=O)(=O)CCN1C1COC1] | 7-(2-((6-(3,6- diazabicyclo[3.1.1]heptan-3-yl)-4- cyclopropylpyridin-3-yl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 385 | 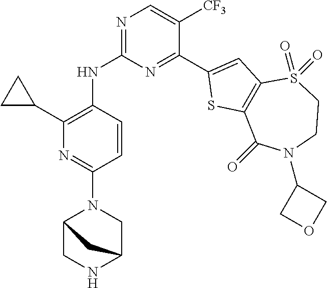 [SMILES: O=C1c2sc(-c3nc(Nc4ccc(N5C[C@H]6C[C@@H]5CN6)nc4C4CC4)ncc3C(F)(F)F)cc2S(=O)(=O)CCN1C1COC1] | 7-(2-((6-((1R,4R)-2,5- diazabicyclo[2.2.1]heptan-2-yl)-2- cyclopropylpyridin-3-yl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 386 | 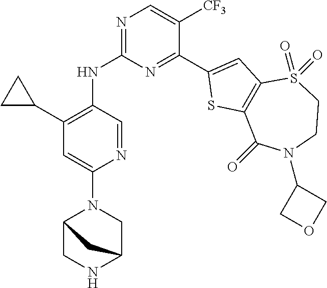 [SMILES: O=C1c2sc(-c3nc(Nc4cnc(N5C[C@H]6C[C@@H]5CN6)cc4C4CC4)ncc3C(F)(F)F)cc2S(=O)(=O)CCN1C1COC1] | 7-(2-((6-((1R,4R)-2,5- diazabicyclo[2.2.1]heptan-2-yl)-4- cyclopropylpyridin-3-yl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 387 | 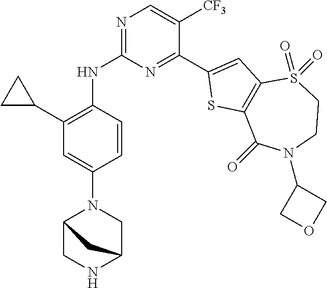 [SMILES: O=C1c2sc(-c3nc(Nc4ccc(N5C[C@H]6C[C@@H]5CN6)cc4C4CC4)ncc3C(F)(F)F)cc2S(=O)(=O)CCN1C1COC1] | 7-(2-((4-((1R,4R)-2,5- diazabicyclo[2.2.1]heptan-2-yl)-2- cyclopropylphenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 388 | 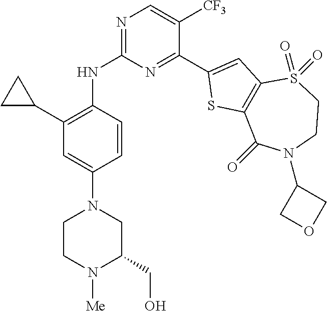 [SMILES: CN1CCN(c2ccc(Nc3ncc(C(F)(F)F)c(-c4cc5c(s4)C(=O)N(C4COC4)CCS5(=O)=O)n3)c(C3CC3)c2)C[C@@H]1CO] | (R)-7-(2-((2-cyclopropyl-4-(3- (hydroxymethyl)-4- methylpiperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 389 | 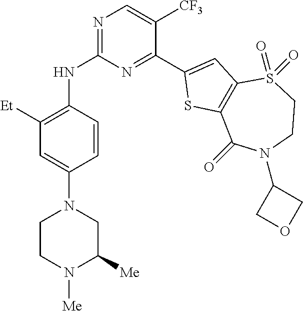 [SMILES: CCc1cc(N2CCN(C)[C@H](C)C2)ccc1Nc1ncc(C(F)(F)F)c(-c2cc3c(s2)C(=O)N(C2COC2)CCS3(=O)=O)n1] | (R)-7-(2-((4-(3,4- dimethylpiperazin-1-yl)-2- ethylphenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 390 | 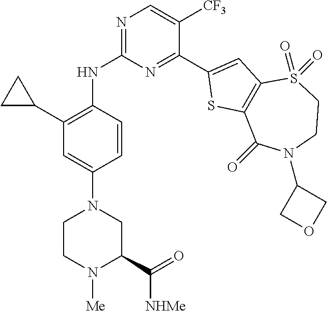 [SMILES: CNC(=O)[C@@H]1CN(c2ccc(Nc3ncc(C(F)(F)F)c(-c4cc5c(s4)C(=O)N(C4COC4)CCS5(=O)=O)n3)c(C3CC3)c2)CCN1C] | (S)-4-(3-cyclopropyl-4-((4-(4- (oxetan-3-yl)-1,1-dioxido-5-oxo- 2,3,4,5-tetrahydrothieno[2,3- f][1,4]thiazepin-7-yl)-5- (trifluoromethyl)pyrimidin-2- yl)amino)phenyl)-N,1- dimethylpiperazine-2-carboxamide |
| 391 | 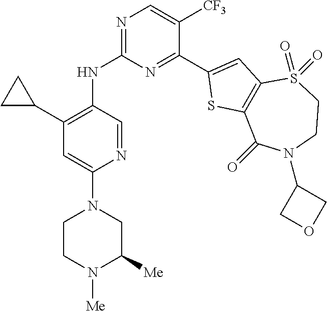 [SMILES: C[C@@H]1CN(c2cc(C3CC3)c(Nc3ncc(C(F)(F)F)c(-c4cc5c(s4)C(=O)N(C4COC4)CCS5(=O)=O)n3)cn2)CCN1C] | (R)-7-(2-((4-cyclopropyl-6-(3,4- dimethylpiperazin-1-yl)pyridin-3- yl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 392 | 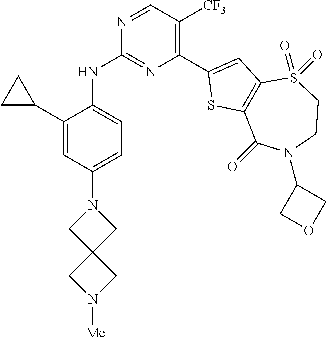 [SMILES: CN1CC2(C1)CN(c1ccc(Nc3ncc(C(F)(F)F)c(-c4cc5c(s4)C(=O)N(C4COC4)CCS5(=O)=O)n3)c(C3CC3)c1)C2] | 7-(2-((2-cyclopropyl-4-(6-methyl- 2,6-diazaspiro[3.3]heptan-2- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 393 | 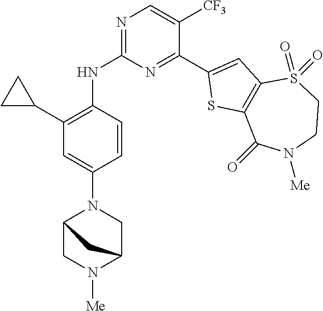 [SMILES: CN1CCS(=O)(=O)c2cc(-c3nc(Nc4ccc(N5C[C@H]6C[C@@H]5CN6C)cc4C4CC4)ncc3C(F)(F)F)sc2C1=O] | 7-(2-((2-cyclopropyl-4-((1R,4R)-5- methyl-2,5- diazabicyclo[2.2.1]heptan-2- yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 394 | 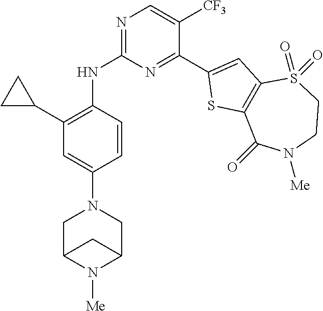 [SMILES: CN1CCS(=O)(=O)c2cc(-c3nc(Nc4ccc(N5CC6CC(C5)N6C)cc4C4CC4)ncc3C(F)(F)F)sc2C1=O] | 7-(2-((2-cyclopropyl-4-(6-methyl- 3,6-diazabicyclo[3.1.1]heptan-3- yl)phenyl)amino)-5- (trifluoromethyl)pyrimidin-4-yl)-4- methyl-3,4-dihydrothieno[2,3- f][1,4]thiazepin-5(2H)-one 1,1- dioxide |
| 395 | 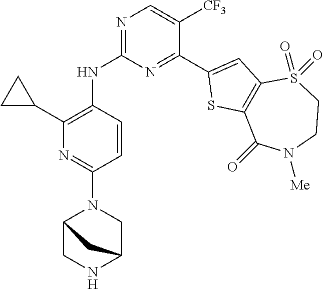 [SMILES: CN1CCS(=O)(=O)c2cc(-c3nc(Nc4ccc(N5C[C@H]6C[C@@H]5CN6)nc4C4CC4)ncc3C(F)(F)F)sc2C1=O] | 7-(2-((6-((1R,4R)-2,5- Diazabicyclo[2.2.1]heptan-2-yl)-2- cyclopropylpyridin-3-yl)amino)-5- (trifluoromethyl)pyrimidin-4-yl)-4- methyl-3,4-dihydrothieno[2,3- f][1,4]thiazepin-5(2H)-one 1,1- dioxide |
| 396 | 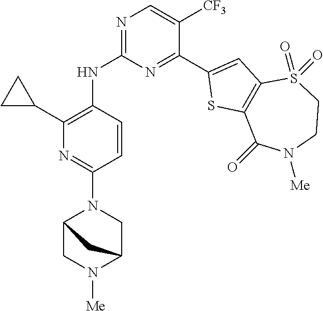 [SMILES: CN1CCS(=O)(=O)c2cc(-c3nc(Nc4ccc(N5C[C@H]6C[C@@H]5CN6C)nc4C4CC4)ncc3C(F)(F)F)sc2C1=O] | 7-(2-((2-Cyclopropyl-6-((1R,4R)-5- methyl-2,5- diazabicyclo[2.2.1]heptan-2- yl)pyridin-3-yl)amino)-5- (trifluoromethyl)pyrimidin-4-yl)-4- methyl-3,4-dihydrothieno[2,3- f][1,4]thiazepin-5(2H)-one 1,1- dioxide |
| 397 | 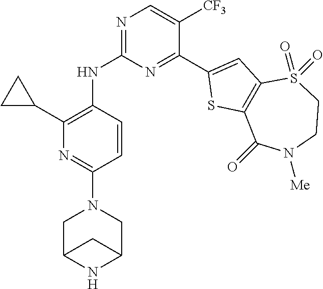 [SMILES: CN1CCS(=O)(=O)c2cc(-c3nc(Nc4ccc(N5CC6CC(C5)N6)nc4C4CC4)ncc3C(F)(F)F)sc2C1=O] | 7-(2-((6-(3,6- diazabicyclo[3.1.1]heptan-3-yl)-2- cyclopropylpyridin-3-yl)amino)-5- (trifluoromethyl)pyrimidin-4-yl)-4- methyl-3,4-dihydrothieno[2,3- f][1,4]thiazepin-5(2H)-one 1,1- dioxide |
| 398 | 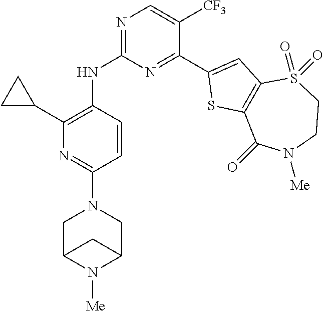 [SMILES: CN1CCS(=O)(=O)c2cc(-c3nc(Nc4ccc(N5CC6CC(C5)N6C)nc4C4CC4)ncc3C(F)(F)F)sc2C1=O] | 7-(2-((2-cyclopropyl-6-(6-methyl- 3,6-diazabicyclo[3.1.1]heptan-3- yl)pyridin-3-yl)amino)-5- (trifluoromethyl)pyrimidin-4-yl)-4- methyl-3,4-dihydrothieno[2,3- f][1,4]thiazepin-5(2H)-one 1,1- dioxide |
| 399 | 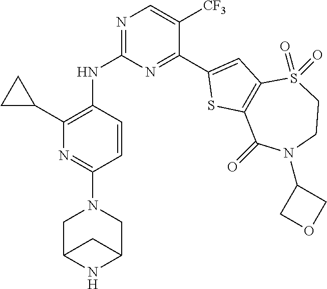 [SMILES: O=C1c2sc(-c3nc(Nc4ccc(N5CC6CC(C5)N6)nc4C4CC4)ncc3C(F)(F)F)cc2S(=O)(=O)CCN1C1COC1] | 7-(2-((6-(3,6- Diazabicyclo[3.1.1]heptan-3-yl)-2- cyclopropylpyridin-3-yl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 400 | 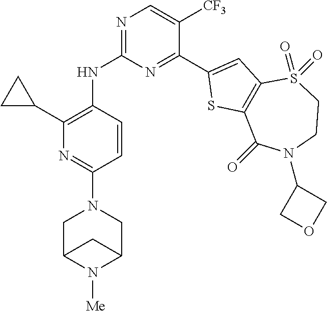 [SMILES: CN1C2CC1CN(c1ccc(Nc3ncc(C(F)(F)F)c(-c4cc5c(s4)C(=O)N(C4COC4)CCS5(=O)=O)n3)c(C3CC3)n1)C2] | 7-(2-((2-Cyclopropyl-6-(6-methyl- 3,6-diazabicyclo[3.1.1]heptan-3- yl)pyridin-3-yl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 401 | 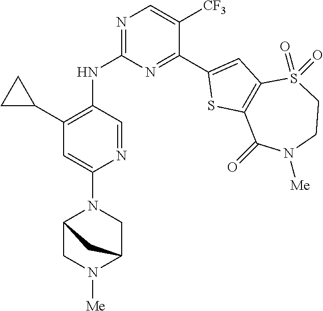 [SMILES: CN1CCS(=O)(=O)c2cc(-c3nc(Nc4cnc(N5C[C@H]6C[C@@H]5CN6C)cc4C4CC4)ncc3C(F)(F)F)sc2C1=O] | 7-(2-((4-cyclopropyl-6-((1R,4R)-5- methyl-2,5- diazabicyclo[2.2.1]heptan-2- yl)pyridin-3-yl)amino)-5- (trifluoromethyl)pyrimidin-4-yl)-4- methyl-3,4-dihydrothieno[2,3- f][1,4]thiazepin-5(2H)-one 1,1- dioxide |
| 402 | 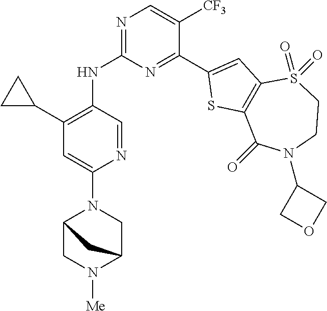 [SMILES: CN1C[C@H]2C[C@@H]1CN2c1cc(C2CC2)c(Nc2ncc(C(F)(F)F)c(-c3cc4c(s3)C(=O)N(C3COC3)CCS4(=O)=O)n2)cn1] | 7-(2-((4-cyclopropyl-6-((1R,4R)-5- methyl-2,5- diazabicyclo[2.2.1]heptan-2- yl)pyridin-3-yl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 403 | 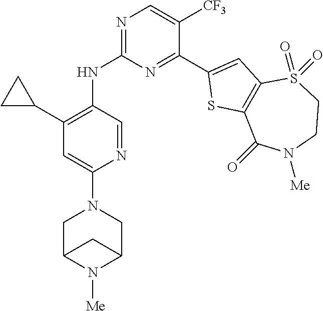 [SMILES: CN1CCS(=O)(=O)c2cc(-c3nc(Nc4cnc(N5CC6CC(C5)N6C)cc4C4CC4)ncc3C(F)(F)F)sc2C1=O] | 7-(2-((4-cyclopropyl-6-(6-methyl- 3,6-diazabicyclo[3.1.1]heptan-3- yl)pyridin-3-yl)amino)-5- (trifluoromethyl)pyrimidin-4-yl)-4- methyl-3,4-dihydrothieno[2,3- f][1,4]thiazepin-5(2H)-one 1,1- dioxide |
| 404 | 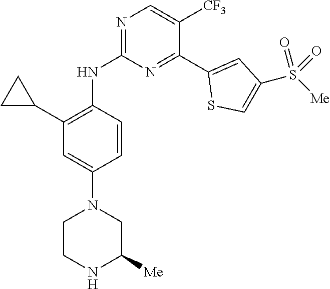 [SMILES: C[C@@H]1CN(c2ccc(Nc3ncc(C(F)(F)F)c(-c4cc(S(C)(=O)=O)cs4)n3)c(C3CC3)c2)CCN1] | (R)-N-(2-cyclopropyl-4-(3- methylpiperazin-1-yl)phenyl)-4-(4- (methylsulfonyl)thiophen-2-yl)-5- (trifluoromethyl)pyrimidin-2-amine |
| 405 | 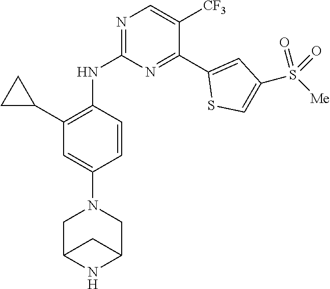 [SMILES: CS(=O)(=O)c1csc(-c2nc(Nc3ccc(N4CC5CC(C4)N5)cc3C3CC3)ncc2C(F)(F)F)c1] | N-(4-(3,6- diazabicyclo[3.1.1]heptan-3-yl)-2- cyclopropylphenyl)-4-(4- (methylsulfonyl)thiophen-2-yl)-5- (trifluoromethyl)pyrimidin-2-amine |
| 406 | 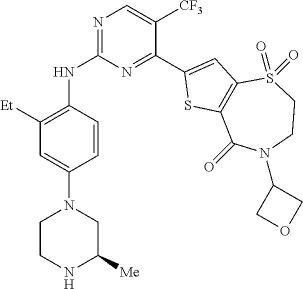 [SMILES: CCc1cc(N2CCN[C@H](C)C2)ccc1Nc1ncc(C(F)(F)F)c(-c2cc3c(s2)C(=O)N(C2COC2)CCS3(=O)=O)n1] | (R)-7-(2-((2-ethyl-4-(3- methylpiperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |
| 407 | 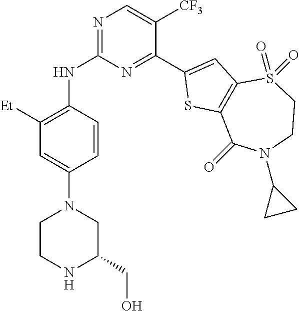 [SMILES: CCc1cc(N2CCN[C@@H](CO)C2)ccc1Nc1ncc(C(F)(F)F)c(-c2cc3c(s2)C(=O)N(C2CC2)CCS3(=O)=O)n1] | (R)-4-cyclopropyl-7-(2-((2-ethyl-4- (3-(hydroxymethyl)piperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 408 | 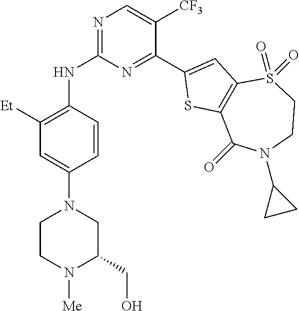 [SMILES: CCc1cc(N2CCN(C)[C@@H](CO)C2)ccc1Nc1ncc(C(F)(F)F)c(-c2cc3c(s2)C(=O)N(C2CC2)CCS3(=O)=O)n1] | (R)-4-cyclopropyl-7-(2-((2-ethyl-4- (3-(hydroxymethyl)-4- methylpiperazin-1- yl)phenyl)amino)-5- (trifluoromethyl)pyrimidin-4-yl)- 3,4-dihydrothieno[2,3- f][1,4]thiazepin-5(2H)-one 1,1- dioxide |
| 409 | 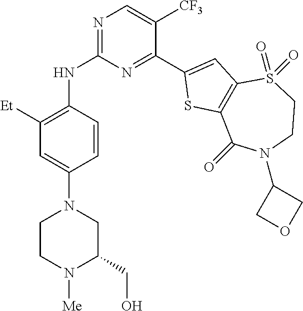 [SMILES: CCc1cc(N2CCN(C)[C@@H](CO)C2)ccc1Nc1ncc(C(F)(F)F)c(-c2cc3c(s2)C(=O)N(C2COC2)CCS3(=O)=O)n1] | (R)-7-(2-((2-Ethyl-4-(3- (hydroxymethyl)-4- methylpiperazin-1- yl)phenyl)amino)-5- (trifluoromethyl)pyrimidin-4-yl)-4- (oxetan-3-yl)-3,4- dihydrothieno[2,3-f][1,4]thiazepin- 5(2H)-one 1,1-dioxide |

In some embodiments, the compound, or a pharmaceutically acceptable salt thereof, is selected from:

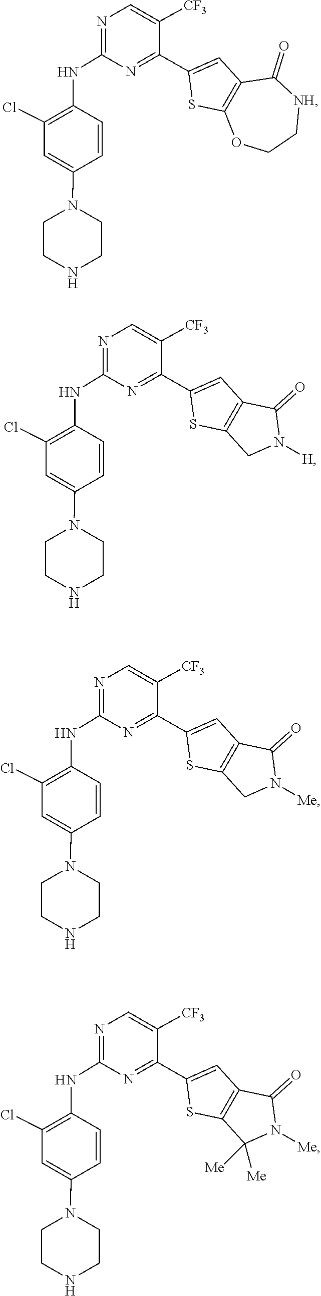

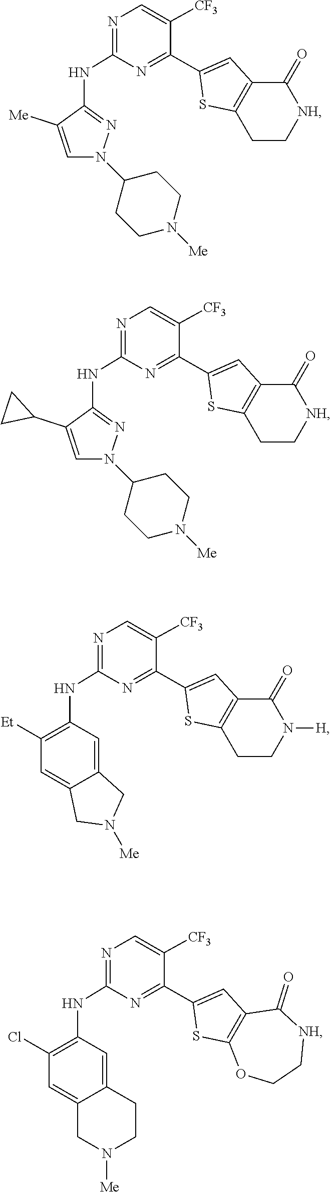

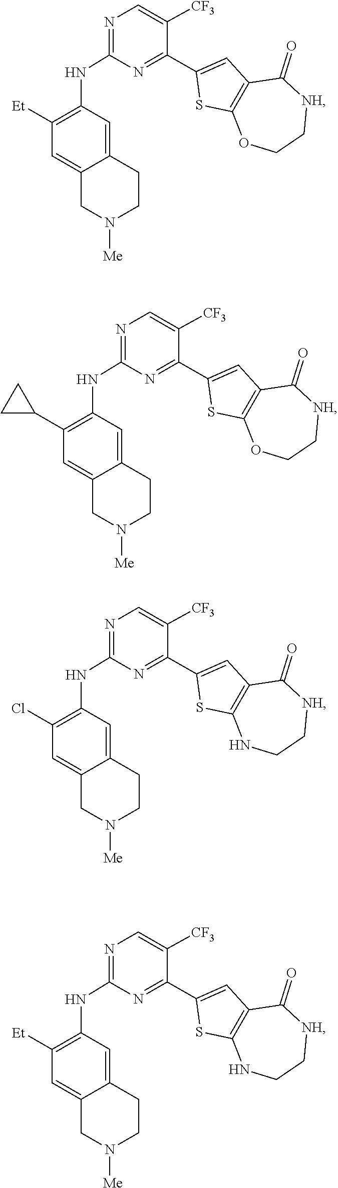 [SMILES: CCc1cc2c(cc1Nc1ncc(C(F)(F)F)c(-c3cc4c(s3)OCCNC4=O)n1)CCN(C)C2 ; CN1CCc2cc(Nc3ncc(C(F)(F)F)c(-c4cc5c(s4)OCCNC5=O)n3)c(C3CC3)cc2C1 ; CN1CCc2cc(Nc3ncc(C(F)(F)F)c(-c4cc5c(s4)NCCNC5=O)n3)c(Cl)cc2C1 ; CCc1cc2c(cc1Nc1ncc(C(F)(F)F)c(-c3cc4c(s3)NCCNC4=O)n1)CCN(C)C2]

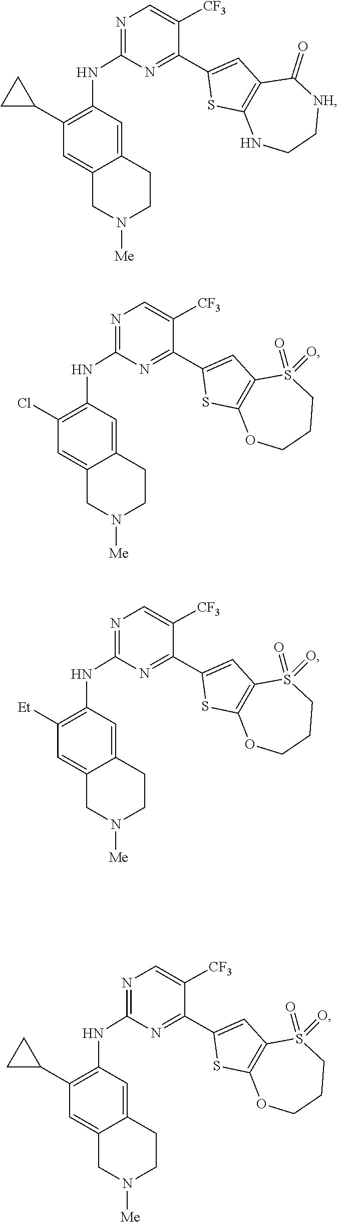 [SMILES: CN1CCc2cc(Nc3ncc(C(F)(F)F)c(-c4cc5c(s4)NCCNC5=O)n3)c(C3CC3)cc2C1 ; CN1CCc2cc(Nc3ncc(C(F)(F)F)c(-c4cc5c(s4)OCCCS5(=O)=O)n3)c(Cl)cc2C1 ; CCc1cc2c(cc1Nc1ncc(C(F)(F)F)c(-c3cc4c(s3)OCCCS4(=O)=O)n1)CCN(C)C2 ; CN1CCc2cc(Nc3ncc(C(F)(F)F)c(-c4cc5c(s4)OCCCS5(=O)=O)n3)c(C3CC3)cc2C1]

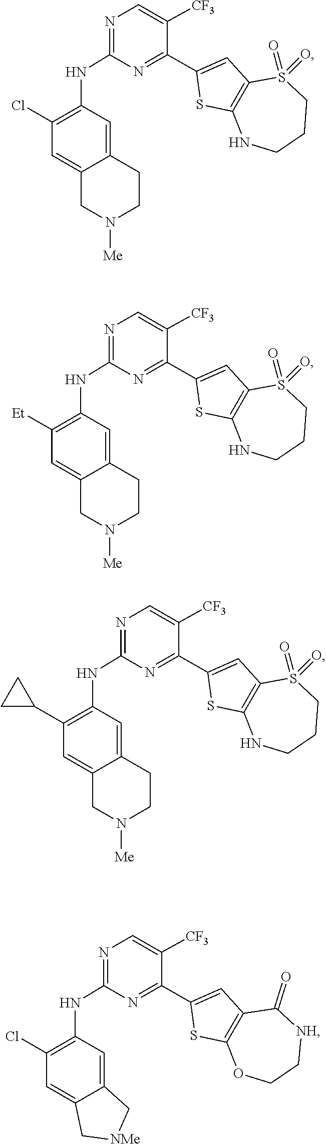 [SMILES: CN1CCc2cc(Nc3ncc(C(F)(F)F)c(-c4cc5c(s4)NCCCS5(=O)=O)n3)c(Cl)cc2C1 ; CCc1cc2c(cc1Nc1ncc(C(F)(F)F)c(-c3cc4c(s3)NCCCS4(=O)=O)n1)CCN(C)C2 ; CN1CCc2cc(Nc3ncc(C(F)(F)F)c(-c4cc5c(s4)NCCCS5(=O)=O)n3)c(C3CC3)cc2C1 ; CN1Cc2cc(Cl)c(Nc3ncc(C(F)(F)F)c(-c4cc5c(s4)OCCNC5=O)n3)cc2C1]

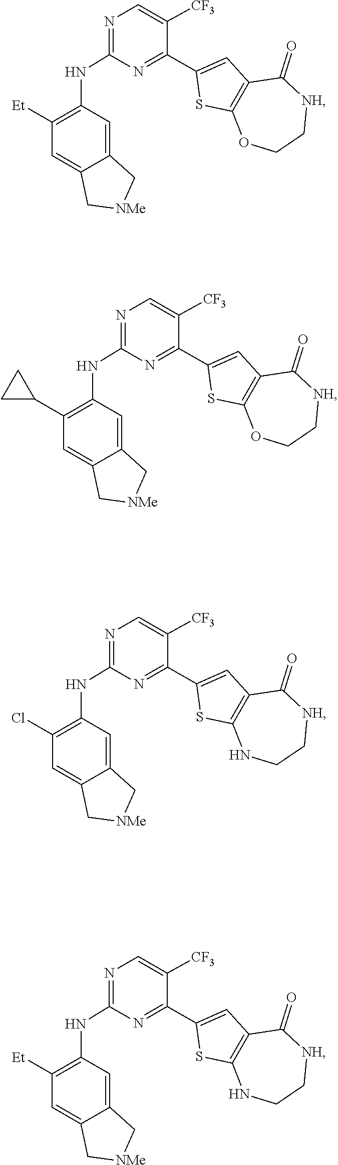 [SMILES: CCc1cc2c(cc1Nc1ncc(C(F)(F)F)c(-c3cc4c(s3)OCCNC4=O)n1)CN(C)C2 ; CN1Cc2cc(Nc3ncc(C(F)(F)F)c(-c4cc5c(s4)OCCNC5=O)n3)c(C3CC3)cc2C1 ; CN1Cc2cc(Cl)c(Nc3ncc(C(F)(F)F)c(-c4cc5c(s4)NCCNC5=O)n3)cc2C1 ; CCc1cc2c(cc1Nc1ncc(C(F)(F)F)c(-c3cc4c(s3)NCCNC4=O)n1)CN(C)C2]

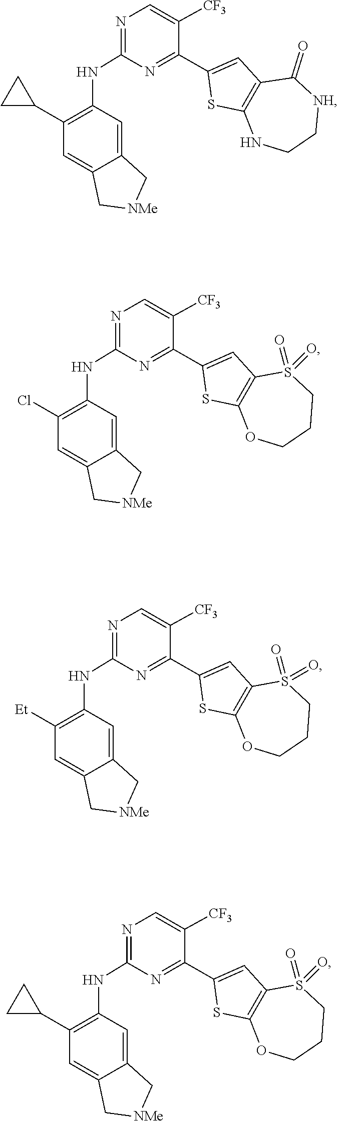 [SMILES: CN1Cc2cc(Nc3ncc(C(F)(F)F)c(-c4cc5c(s4)NCCNC5=O)n3)c(C3CC3)cc2C1 ; CN1Cc2cc(Cl)c(Nc3ncc(C(F)(F)F)c(-c4cc5c(s4)OCCCS5(=O)=O)n3)cc2C1 ; CCc1cc2c(cc1Nc1ncc(C(F)(F)F)c(-c3cc4c(s3)OCCCS4(=O)=O)n1)CN(C)C2 ; CN1Cc2cc(Nc3ncc(C(F)(F)F)c(-c4cc5c(s4)OCCCS5(=O)=O)n3)c(C3CC3)cc2C1]

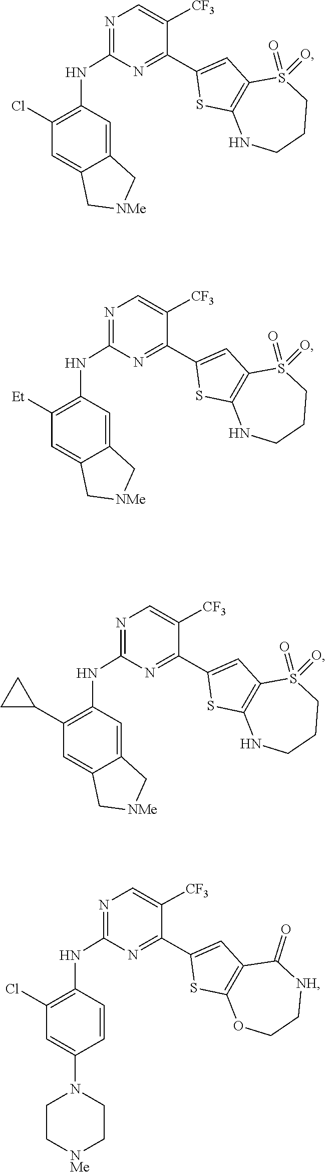 [SMILES: CN1Cc2cc(Cl)c(Nc3ncc(C(F)(F)F)c(-c4cc5c(s4)NCCCS5(=O)=O)n3)cc2C1 ; CCc1cc2c(cc1Nc1ncc(C(F)(F)F)c(-c3cc4c(s3)NCCCS4(=O)=O)n1)CN(C)C2 ; CN1Cc2cc(Nc3ncc(C(F)(F)F)c(-c4cc5c(s4)NCCCS5(=O)=O)n3)c(C3CC3)cc2C1 ; CN1CCN(c2ccc(Nc3ncc(C(F)(F)F)c(-c4cc5c(s4)OCCNC5=O)n3)c(Cl)c2)CC1]

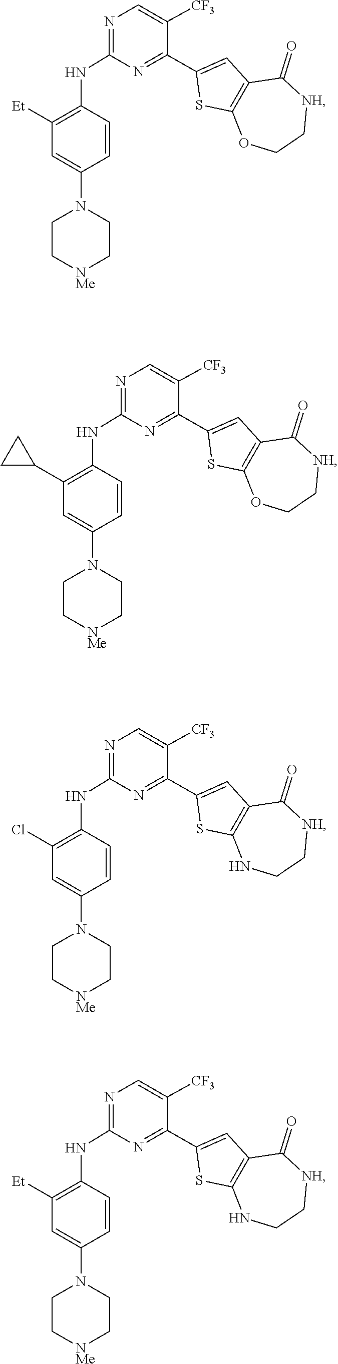 [SMILES: CCc1cc(N2CCN(C)CC2)ccc1Nc1ncc(C(F)(F)F)c(-c2cc3c(s2)OCCNC3=O)n1 ; CN1CCN(c2ccc(Nc3ncc(C(F)(F)F)c(-c4cc5c(s4)OCCNC5=O)n3)c(C3CC3)c2)CC1 ; CN1CCN(c2ccc(Nc3ncc(C(F)(F)F)c(-c4cc5c(s4)NCCNC5=O)n3)c(Cl)c2)CC1 ; CCc1cc(N2CCN(C)CC2)ccc1Nc1ncc(C(F)(F)F)c(-c2cc3c(s2)NCCNC3=O)n1]

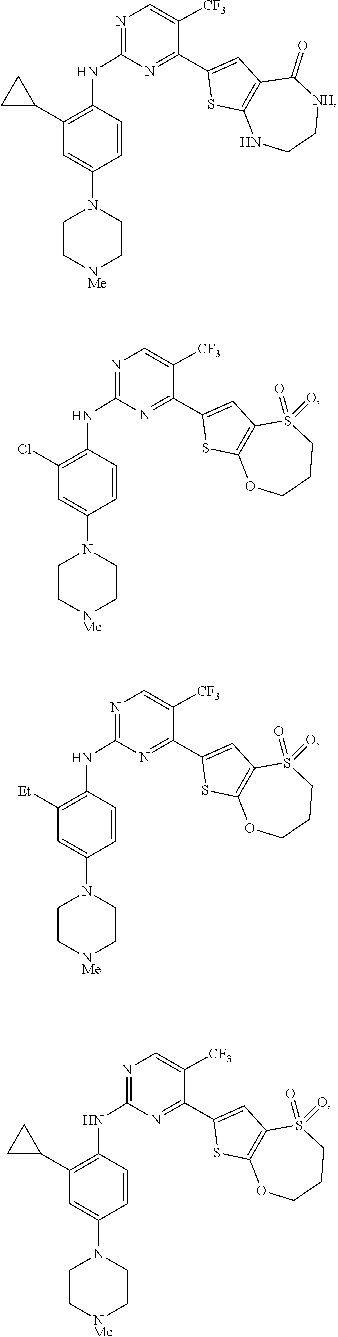 [SMILES: CN1CCN(c2ccc(Nc3ncc(C(F)(F)F)c(-c4cc5c(s4)NCCNC5=O)n3)c(C3CC3)c2)CC1 ; CN1CCN(c2ccc(Nc3ncc(C(F)(F)F)c(-c4cc5c(s4)OCCCS5(=O)=O)n3)c(Cl)c2)CC1 ; CCc1cc(N2CCN(C)CC2)ccc1Nc1ncc(C(F)(F)F)c(-c2cc3c(s2)OCCCS3(=O)=O)n1 ; CN1CCN(c2ccc(Nc3ncc(C(F)(F)F)c(-c4cc5c(s4)OCCCS5(=O)=O)n3)c(C3CC3)c2)CC1]

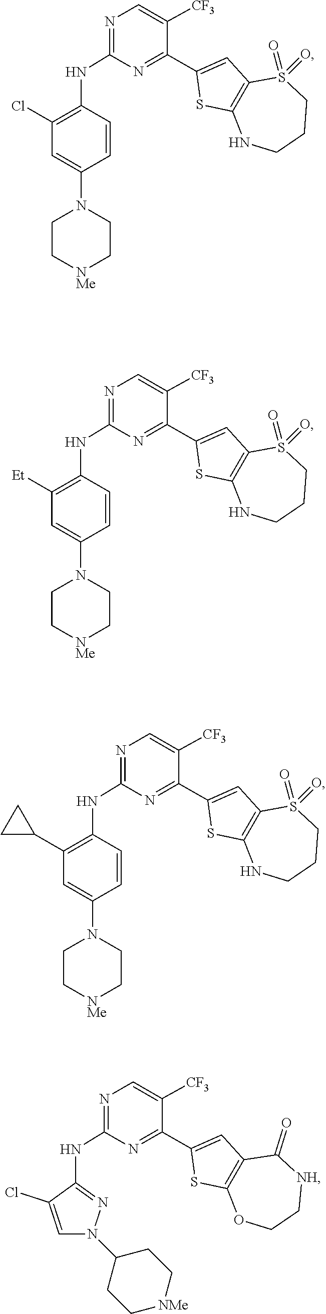 [SMILES: CN1CCN(c2ccc(Nc3ncc(C(F)(F)F)c(-c4cc5c(s4)NCCCS5(=O)=O)n3)c(Cl)c2)CC1 ; CCc1cc(N2CCN(C)CC2)ccc1Nc1ncc(C(F)(F)F)c(-c2cc3c(s2)NCCCS3(=O)=O)n1 ; CN1CCN(c2ccc(Nc3ncc(C(F)(F)F)c(-c4cc5c(s4)NCCCS5(=O)=O)n3)c(C3CC3)c2)CC1 ; CN1CCC(n2cc(Cl)c(Nc3ncc(C(F)(F)F)c(-c4cc5c(s4)OCCNC5=O)n3)n2)CC1]

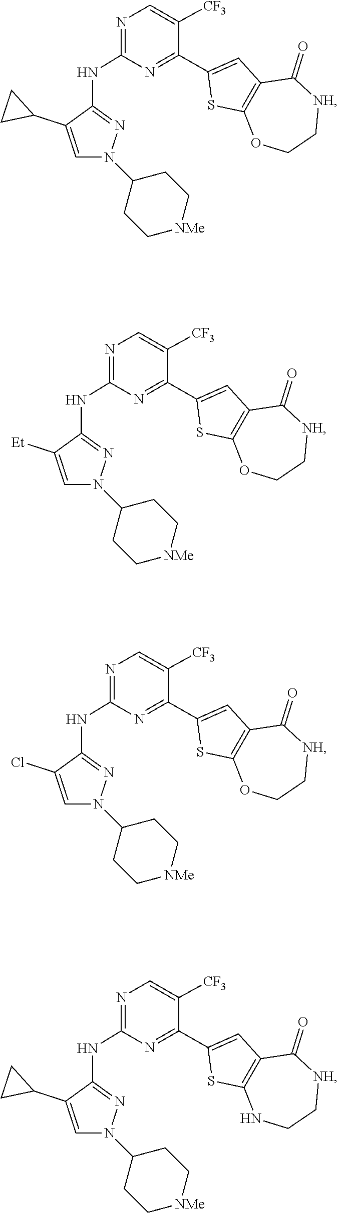 [SMILES: CN1CCC(n2cc(C3CC3)c(Nc3ncc(C(F)(F)F)c(-c4cc5c(s4)OCCNC5=O)n3)n2)CC1 ; CCc1cn(C2CCN(C)CC2)nc1Nc1ncc(C(F)(F)F)c(-c2cc3c(s2)OCCNC3=O)n1 ; CN1CCC(n2cc(Cl)c(Nc3ncc(C(F)(F)F)c(-c4cc5c(s4)OCCNC5=O)n3)n2)CC1 ; CN1CCC(n2cc(C3CC3)c(Nc3ncc(C(F)(F)F)c(-c4cc5c(s4)NCCNC5=O)n3)n2)CC1]

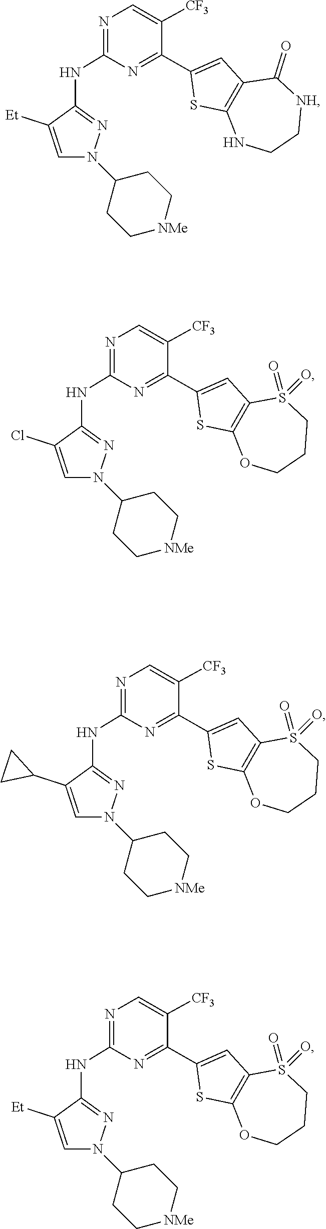 [SMILES: CCc1cn(C2CCN(C)CC2)nc1Nc1ncc(C(F)(F)F)c(-c2cc3c(s2)NCCNC3=O)n1 ; CN1CCC(n2cc(Cl)c(Nc3ncc(C(F)(F)F)c(-c4cc5c(s4)OCCCS5(=O)=O)n3)n2)CC1 ; CN1CCC(n2cc(C3CC3)c(Nc3ncc(C(F)(F)F)c(-c4cc5c(s4)OCCCS5(=O)=O)n3)n2)CC1 ; CCc1cn(C2CCN(C)CC2)nc1Nc1ncc(C(F)(F)F)c(-c2cc3c(s2)OCCCS3(=O)=O)n1]

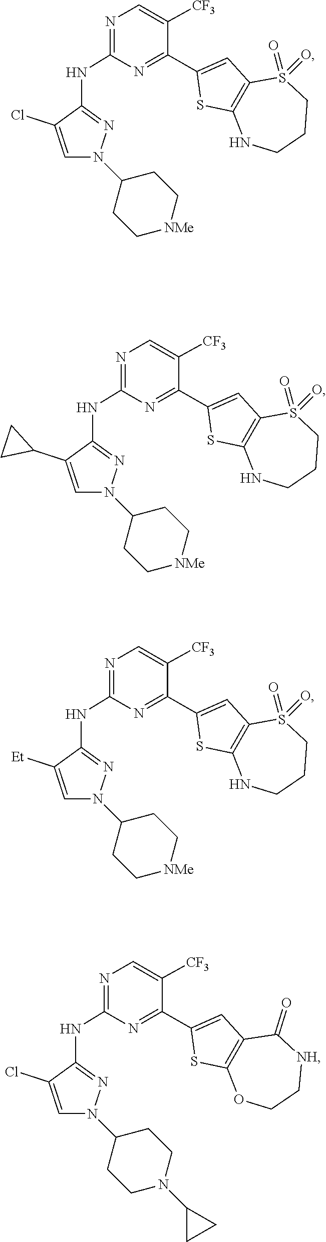 [SMILES: CN1CCC(n2cc(Cl)c(Nc3ncc(C(F)(F)F)c(-c4cc5c(s4)NCCCS5(=O)=O)n3)n2)CC1 ; CN1CCC(n2cc(C3CC3)c(Nc3ncc(C(F)(F)F)c(-c4cc5c(s4)NCCCS5(=O)=O)n3)n2)CC1 ; CCc1cn(C2CCN(C)CC2)nc1Nc1ncc(C(F)(F)F)c(-c2cc3c(s2)NCCCS3(=O)=O)n1 ; O=C1NCCOc2sc(-c3nc(Nc4nn(C5CCN(C6CC6)CC5)cc4Cl)ncc3C(F)(F)F)cc21]

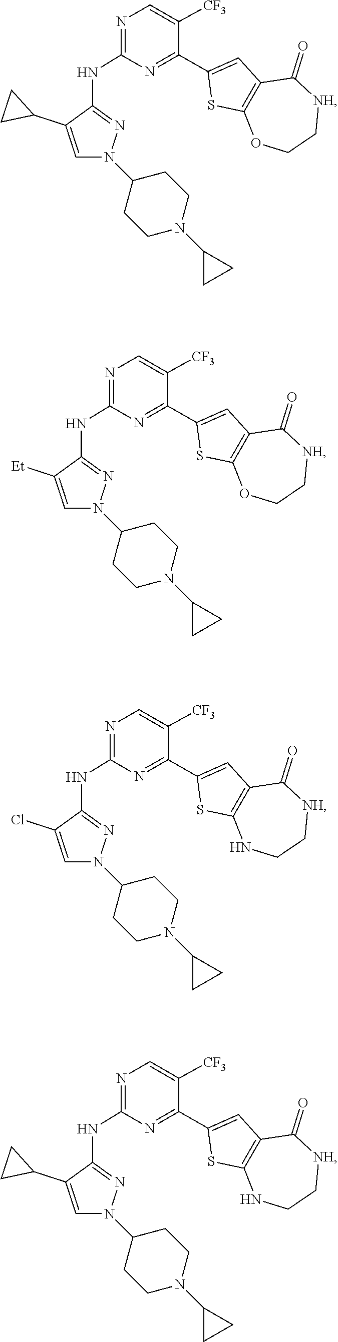 [SMILES: O=C1NCCOc2sc(-c3nc(Nc4nn(C5CCN(C6CC6)CC5)cc4C4CC4)ncc3C(F)(F)F)cc21 ; CCc1cn(C2CCN(C3CC3)CC2)nc1Nc1ncc(C(F)(F)F)c(-c2cc3c(s2)OCCNC3=O)n1 ; O=C1NCCNc2sc(-c3nc(Nc4nn(C5CCN(C6CC6)CC5)cc4Cl)ncc3C(F)(F)F)cc21 ; O=C1NCCNc2sc(-c3nc(Nc4nn(C5CCN(C6CC6)CC5)cc4C4CC4)ncc3C(F)(F)F)cc21]

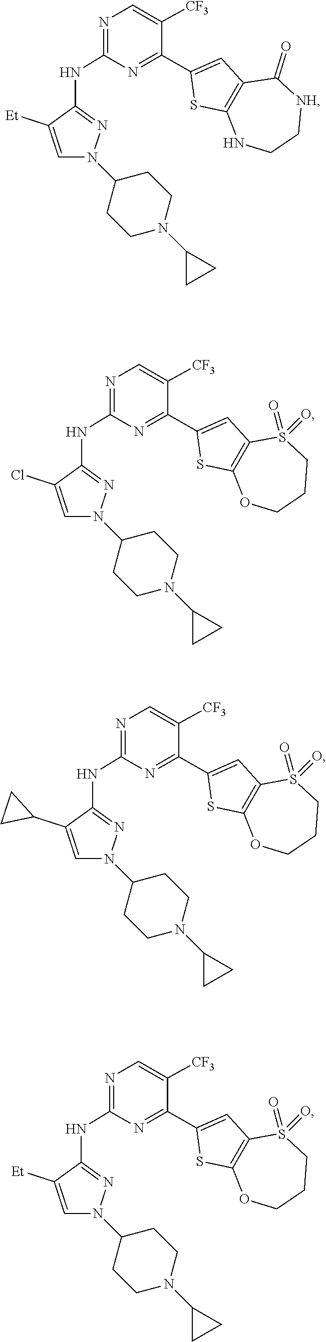 [SMILES: CCc1cn(C2CCN(C3CC3)CC2)nc1Nc1ncc(C(F)(F)F)c(-c2cc3c(s2)NCCNC3=O)n1 ; O=S1(=O)CCCOc2sc(-c3nc(Nc4nn(C5CCN(C6CC6)CC5)cc4Cl)ncc3C(F)(F)F)cc21 ; O=S1(=O)CCCOc2sc(-c3nc(Nc4nn(C5CCN(C6CC6)CC5)cc4C4CC4)ncc3C(F)(F)F)cc21 ; CCc1cn(C2CCN(C3CC3)CC2)nc1Nc1ncc(C(F)(F)F)c(-c2cc3c(s2)OCCCS3(=O)=O)n1]

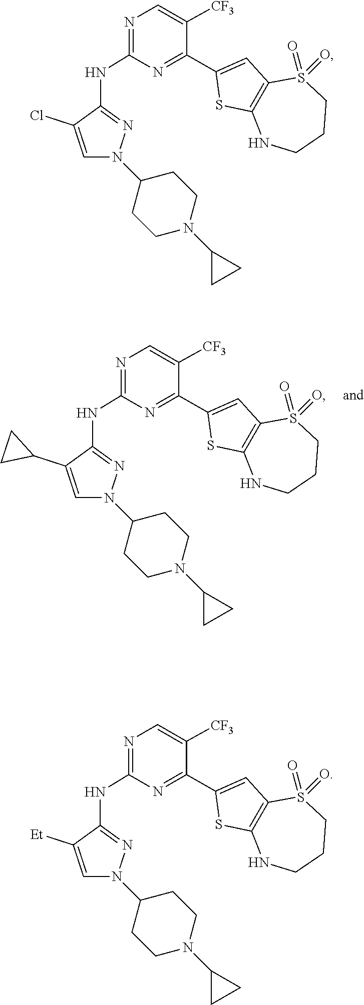 [SMILES: O=S1(=O)CCCNc2sc(-c3nc(Nc4nn(C5CCN(C6CC6)CC5)cc4Cl)ncc3C(F)(F)F)cc21 ; O=S1(=O)CCCNc2sc(-c3nc(Nc4nn(C5CCN(C6CC6)CC5)cc4C4CC4)ncc3C(F)(F)F)cc21 ; "and" ; CCc1cn(C2CCN(C3CC3)CC2)nc1Nc1ncc(C(F)(F)F)c(-c2cc3c(s2)NCCCS3(=O)=O)n1]

In some embodiments, the compound, or a pharmaceutically acceptable salt thereof, is selected from:

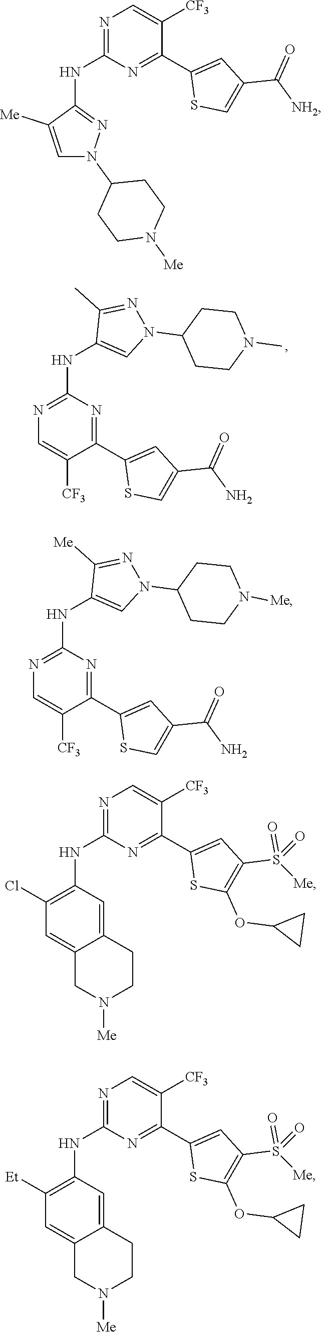

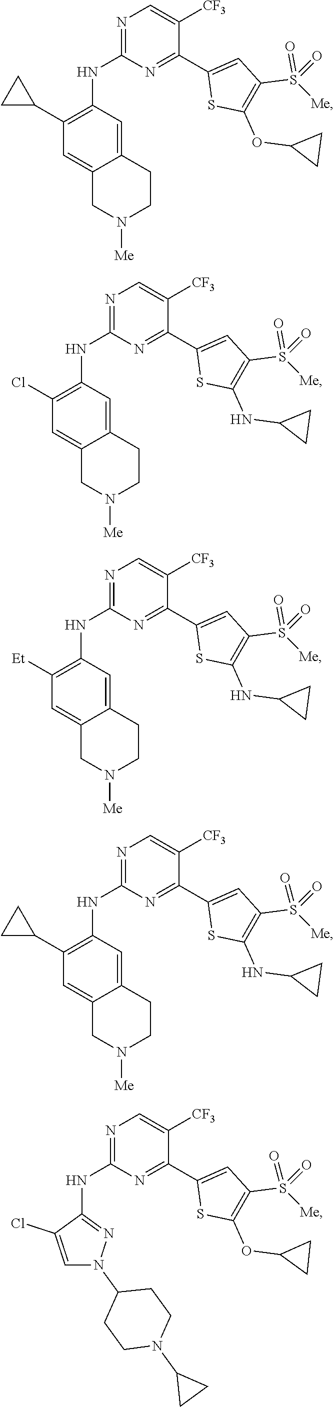

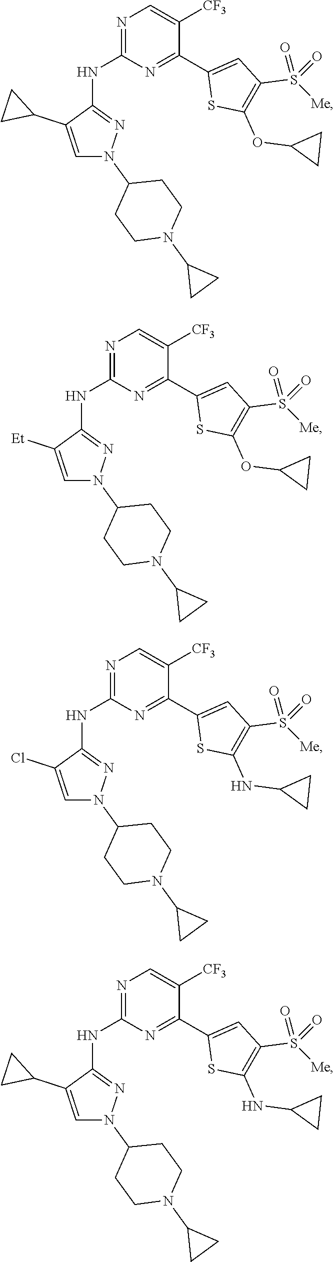

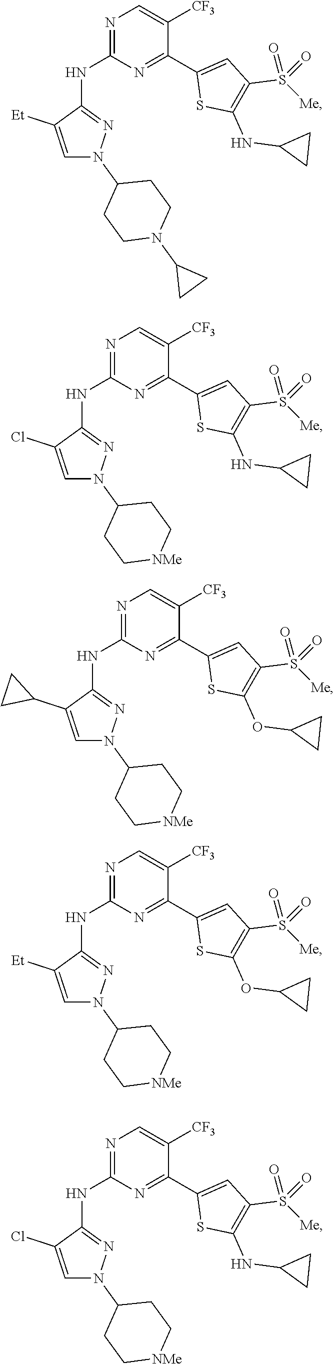

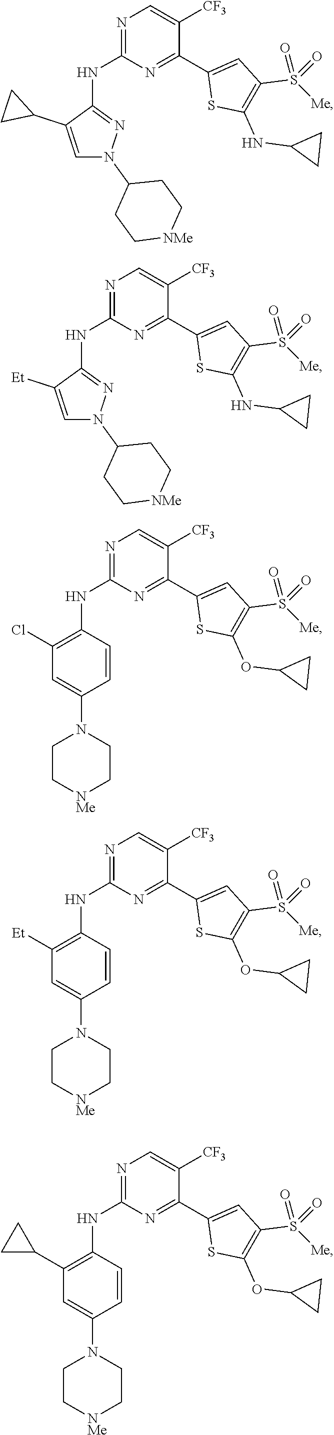

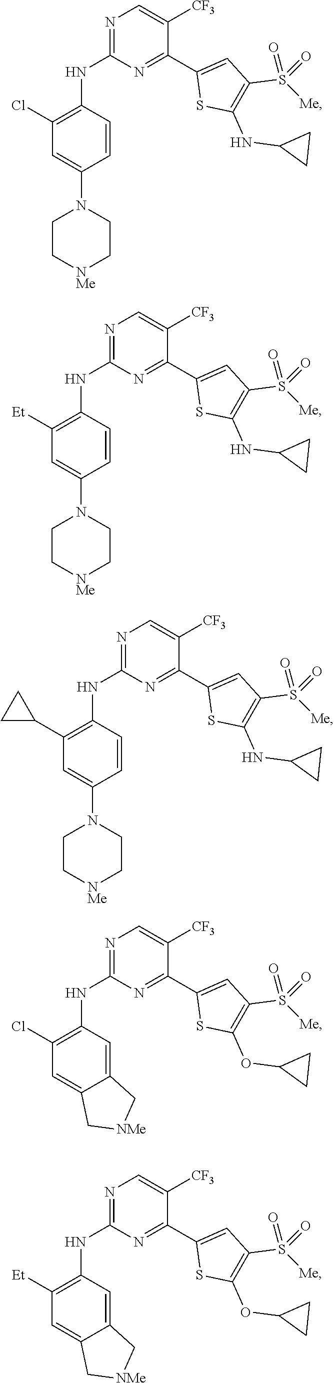 [SMILES: CN1CCN(c2ccc(Nc3ncc(C(F)(F)F)c(-c4cc(S(C)(=O)=O)c(NC5CC5)s4)n3)c(Cl)c2)CC1 ; CCc1cc(N2CCN(C)CC2)ccc1Nc1ncc(C(F)(F)F)c(-c2cc(S(C)(=O)=O)c(NC3CC3)s2)n1 ; CN1CCN(c2ccc(Nc3ncc(C(F)(F)F)c(-c4cc(S(C)(=O)=O)c(NC5CC5)s4)n3)c(C3CC3)c2)CC1 ; CN1Cc2cc(Cl)c(Nc3ncc(C(F)(F)F)c(-c4cc(S(C)(=O)=O)c(OC5CC5)s4)n3)cc2C1 ; CCc1cc2c(cc1Nc1ncc(C(F)(F)F)c(-c3cc(S(C)(=O)=O)c(OC4CC4)s3)n1)CN(C)C2]

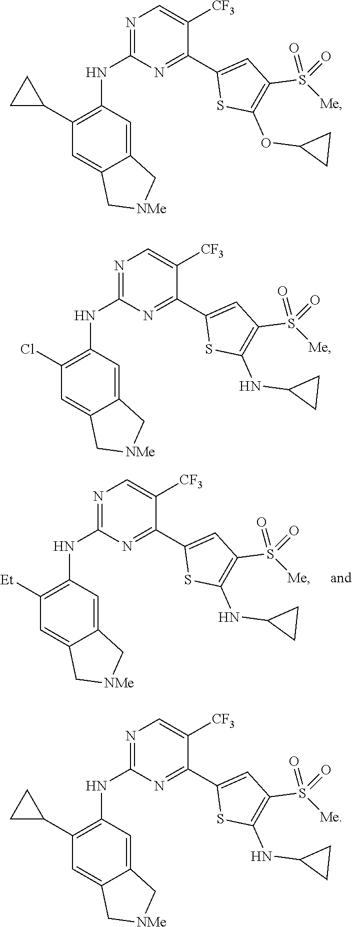

In some embodiments, the compound, or a pharmaceutically acceptable salt thereof, is selected from:

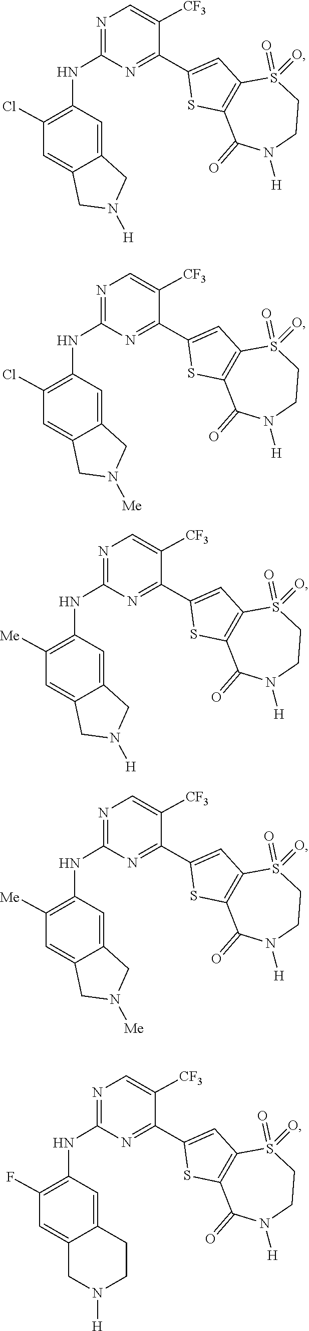

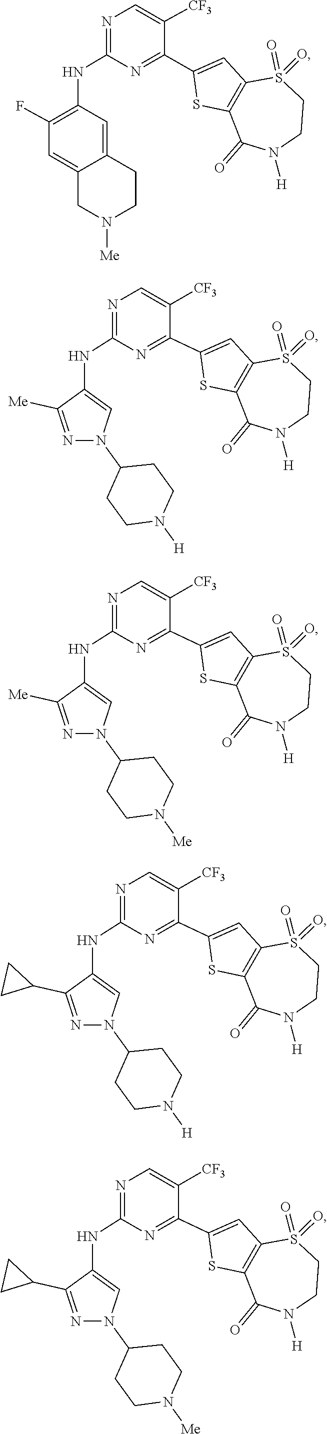

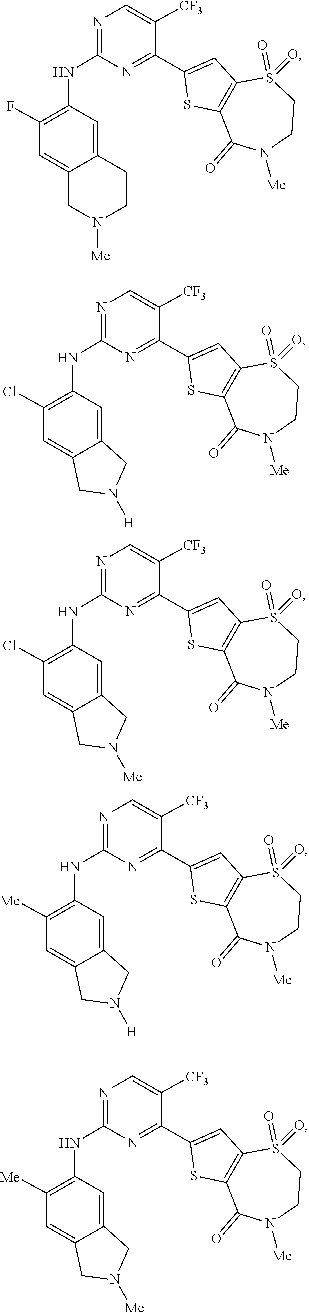

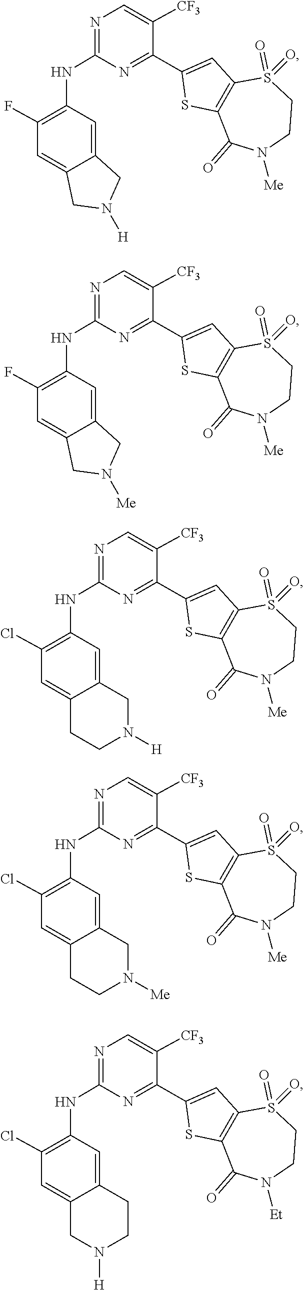

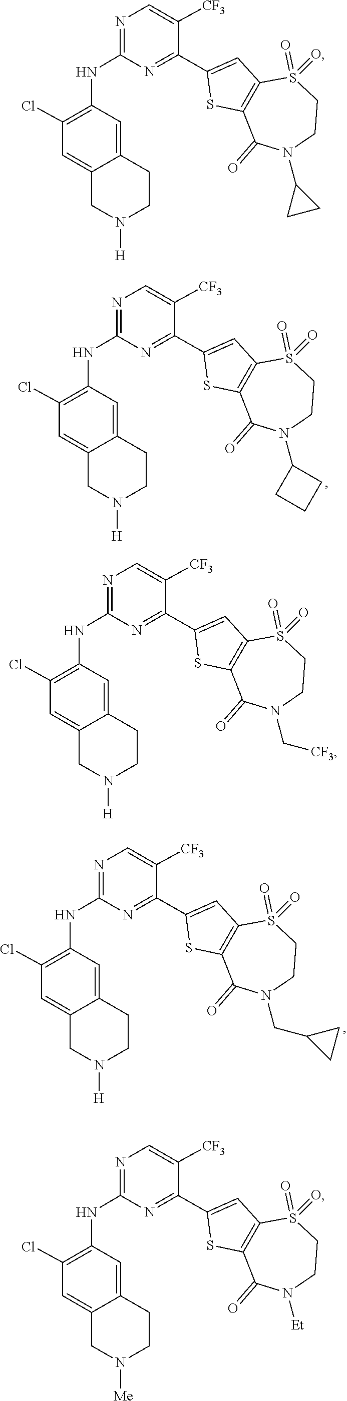

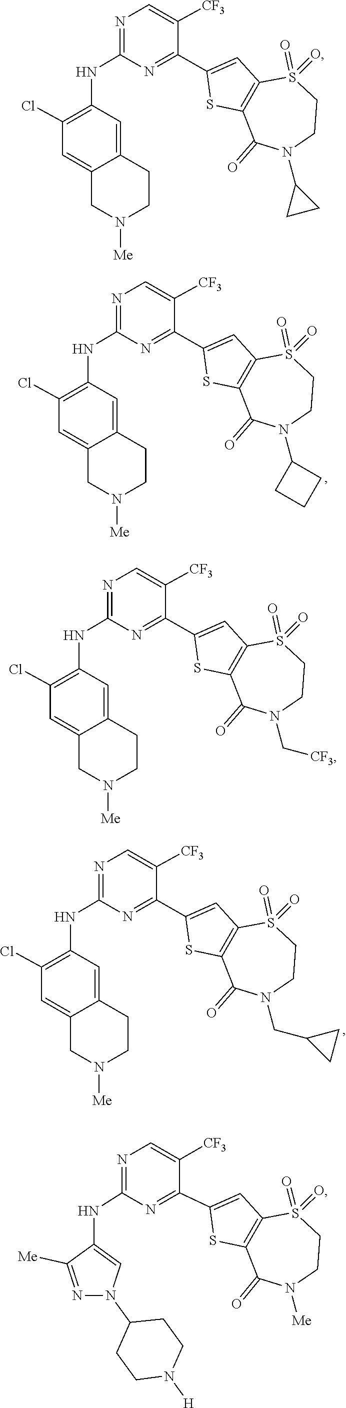

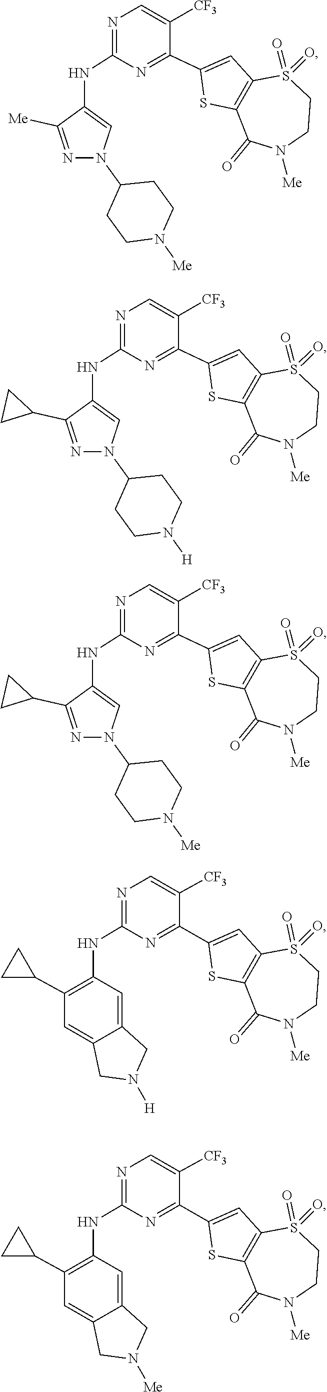

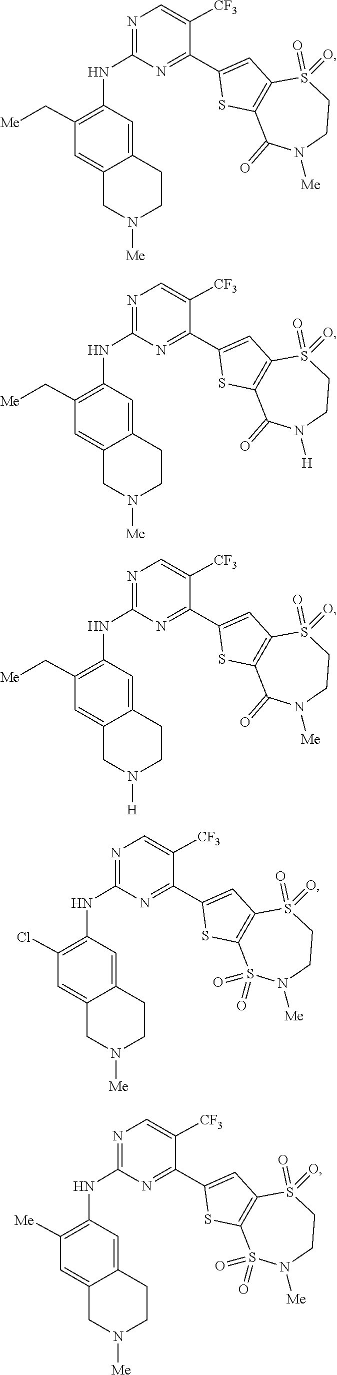

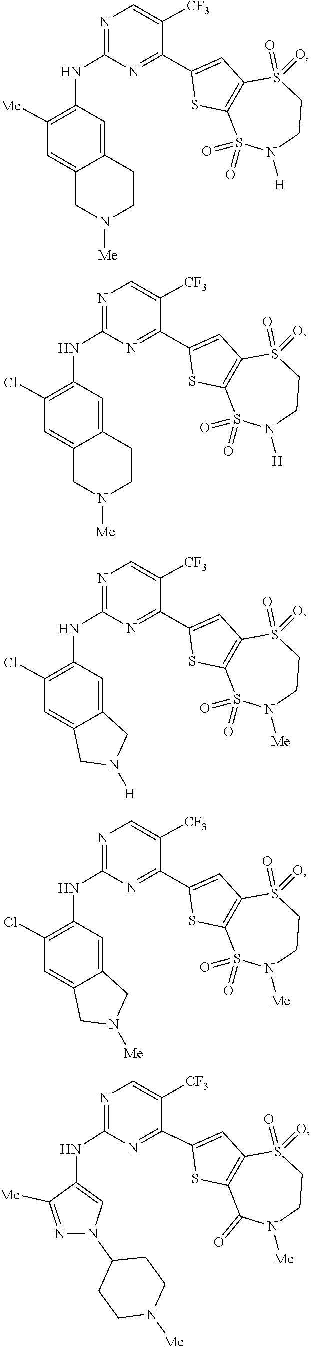

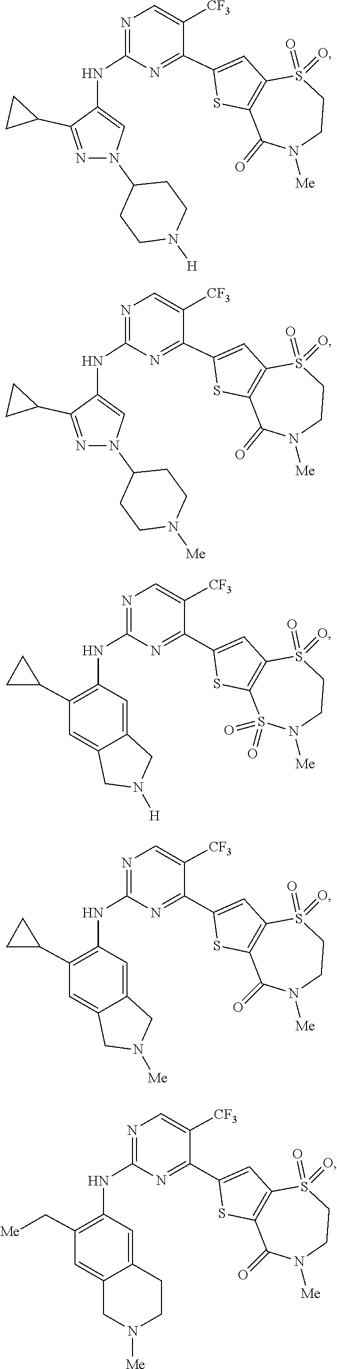

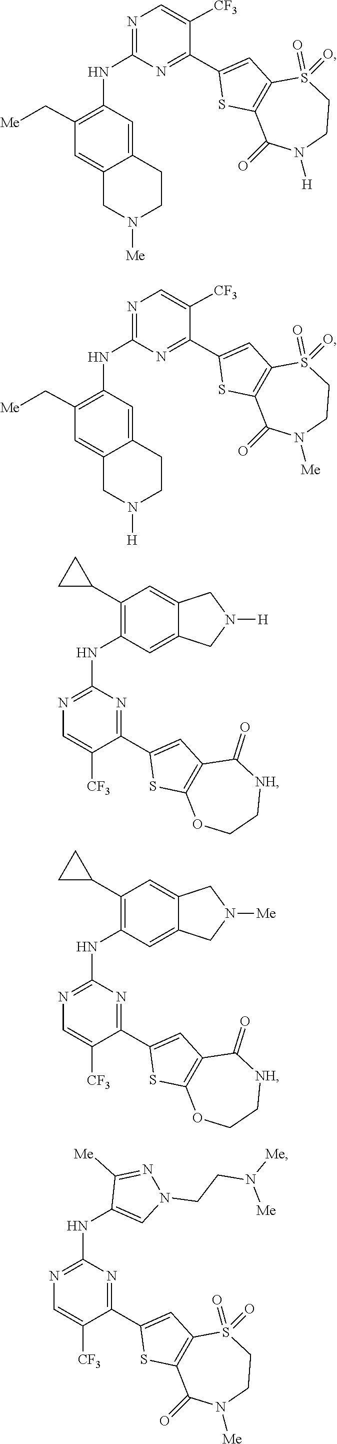

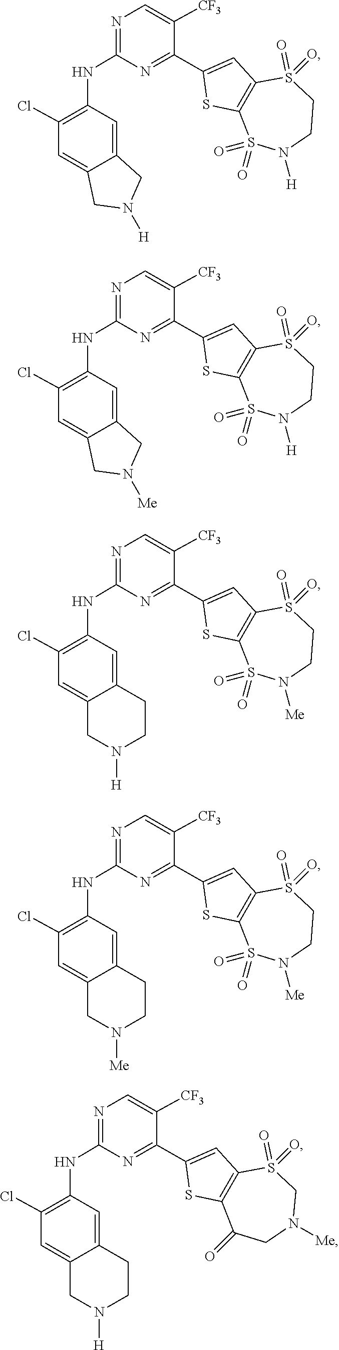

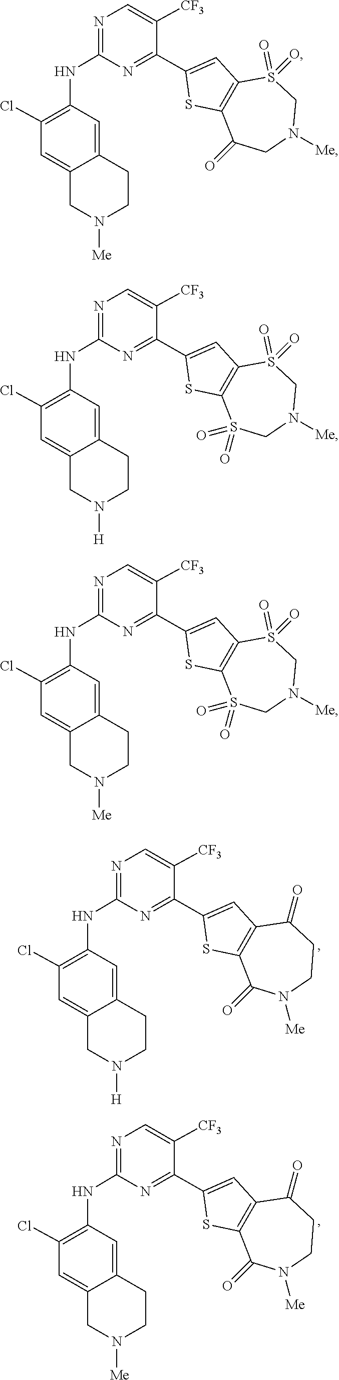

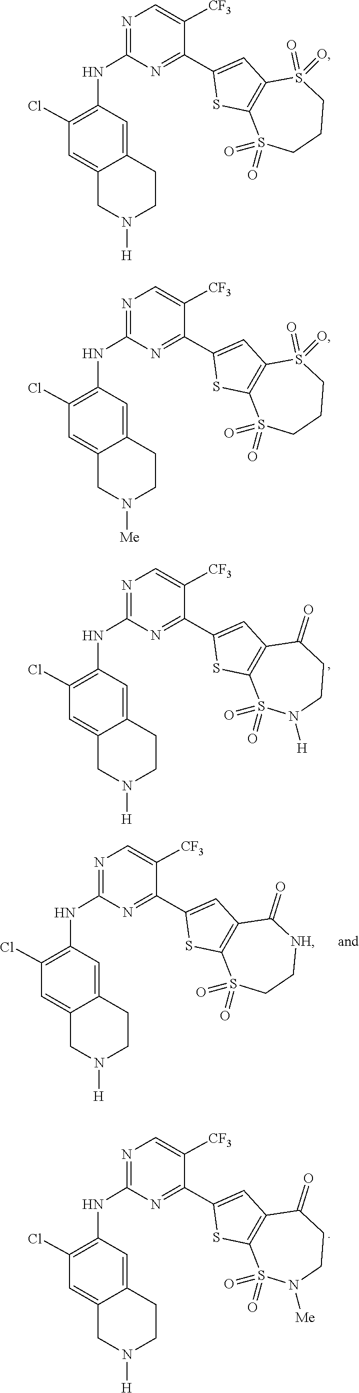

In some embodiments, the compound, or a pharmaceutically acceptable salt thereof, is selected from:

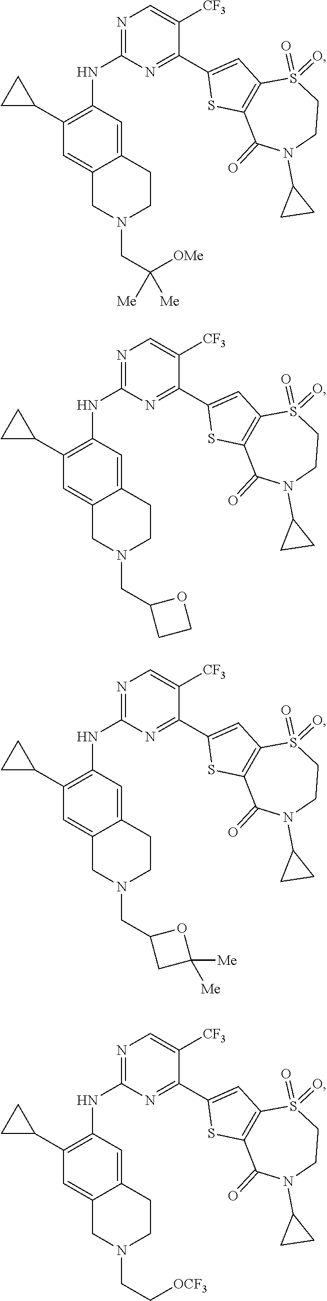

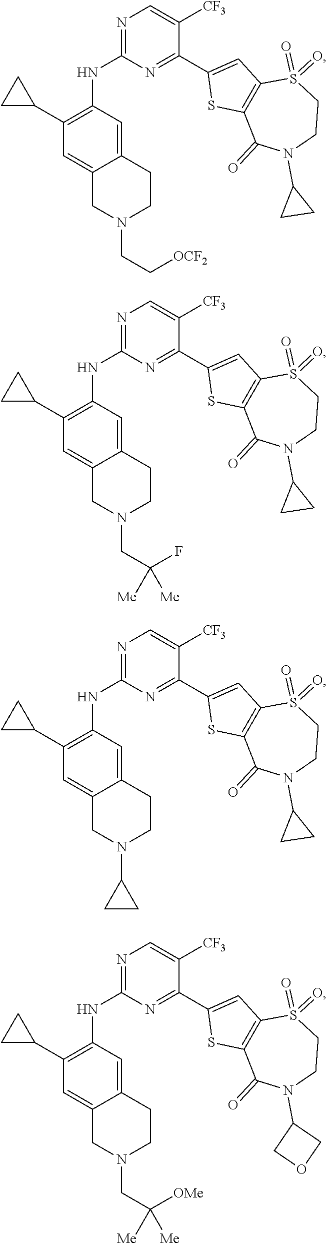

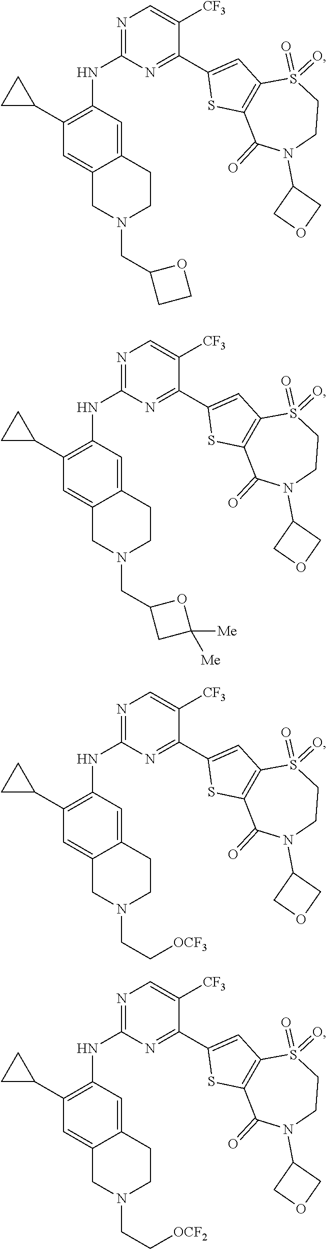

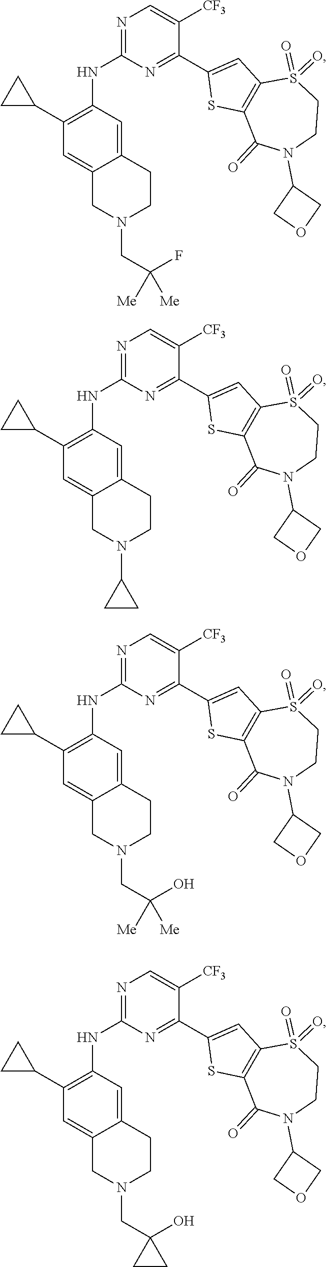

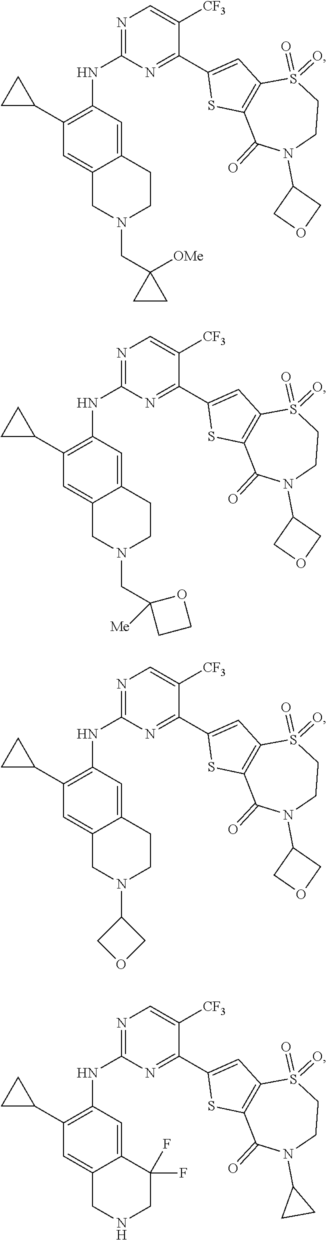

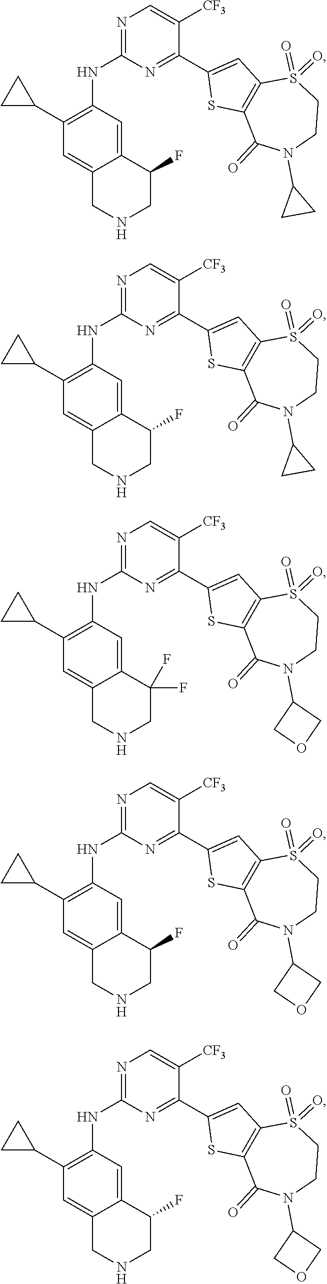 [SMILES: O=C1c2sc(-c3nc(Nc4cc5c(cc4C4CC4)CNC[C@@H]5F)ncc3C(F)(F)F)cc2S(=O)(=O)CCN1C1CC1 ; O=C1c2sc(-c3nc(Nc4cc5c(cc4C4CC4)CNC[C@H]5F)ncc3C(F)(F)F)cc2S(=O)(=O)CCN1C1CC1 ; O=C1c2sc(-c3nc(Nc4cc5c(cc4C4CC4)CNCC5(F)F)ncc3C(F)(F)F)cc2S(=O)(=O)CCN1C1COC1 ; O=C1c2sc(-c3nc(Nc4cc5c(cc4C4CC4)CNC[C@@H]5F)ncc3C(F)(F)F)cc2S(=O)(=O)CCN1C1COC1 ; O=C1c2sc(-c3nc(Nc4cc5c(cc4C4CC4)CNC[C@H]5F)ncc3C(F)(F)F)cc2S(=O)(=O)CCN1C1COC1]

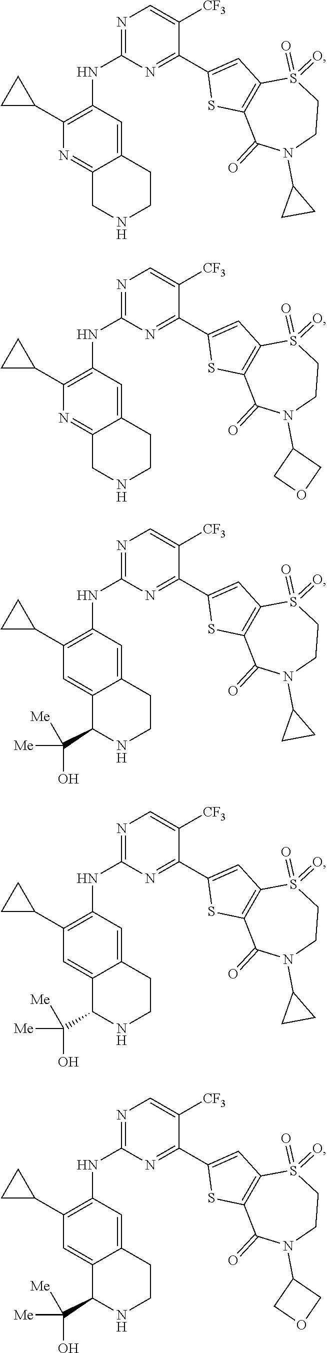 [SMILES: O=C1c2sc(-c3nc(Nc4cc5c(nc4C4CC4)CNCC5)ncc3C(F)(F)F)cc2S(=O)(=O)CCN1C1CC1 ; O=C1c2sc(-c3nc(Nc4cc5c(nc4C4CC4)CNCC5)ncc3C(F)(F)F)cc2S(=O)(=O)CCN1C1COC1 ; CC(C)(O)[C@@H]1NCCc2cc(Nc3ncc(C(F)(F)F)c(-c4cc5c(s4)C(=O)N(C4CC4)CCS5(=O)=O)n3)c(C3CC3)cc21 ; CC(C)(O)[C@H]1NCCc2cc(Nc3ncc(C(F)(F)F)c(-c4cc5c(s4)C(=O)N(C4CC4)CCS5(=O)=O)n3)c(C3CC3)cc21 ; CC(C)(O)[C@@H]1NCCc2cc(Nc3ncc(C(F)(F)F)c(-c4cc5c(s4)C(=O)N(C4COC4)CCS5(=O)=O)n3)c(C3CC3)cc21]

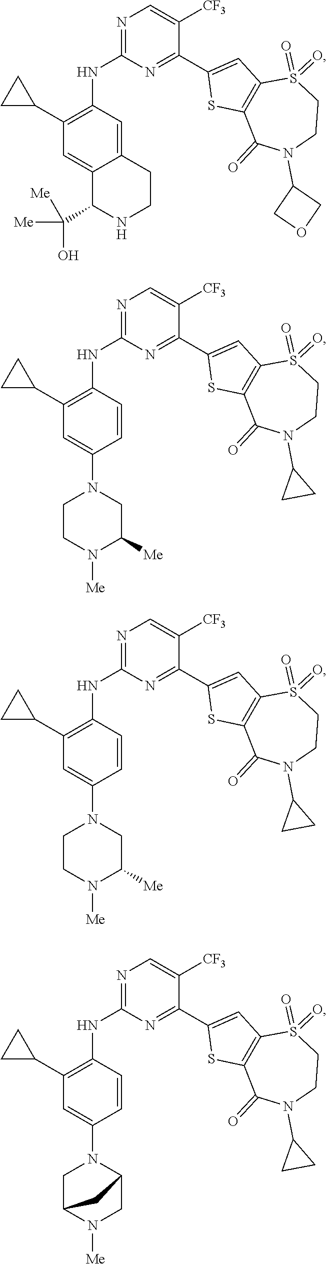 [SMILES: CC(C)(O)[C@H]1NCCc2cc(Nc3ncc(C(F)(F)F)c(-c4cc5c(s4)C(=O)N(C4COC4)CCS5(=O)=O)n3)c(C3CC3)cc21 ; C[C@@H]1CN(c2ccc(Nc3ncc(C(F)(F)F)c(-c4cc5c(s4)C(=O)N(C4CC4)CCS5(=O)=O)n3)c(C3CC3)c2)CCN1C ; C[C@H]1CN(c2ccc(Nc3ncc(C(F)(F)F)c(-c4cc5c(s4)C(=O)N(C4CC4)CCS5(=O)=O)n3)c(C3CC3)c2)CCN1C ; CN1C[C@@H]2C[C@H]1CN2c1ccc(Nc2ncc(C(F)(F)F)c(-c3cc4c(s3)C(=O)N(C3CC3)CCS4(=O)=O)n2)c(C2CC2)c1]

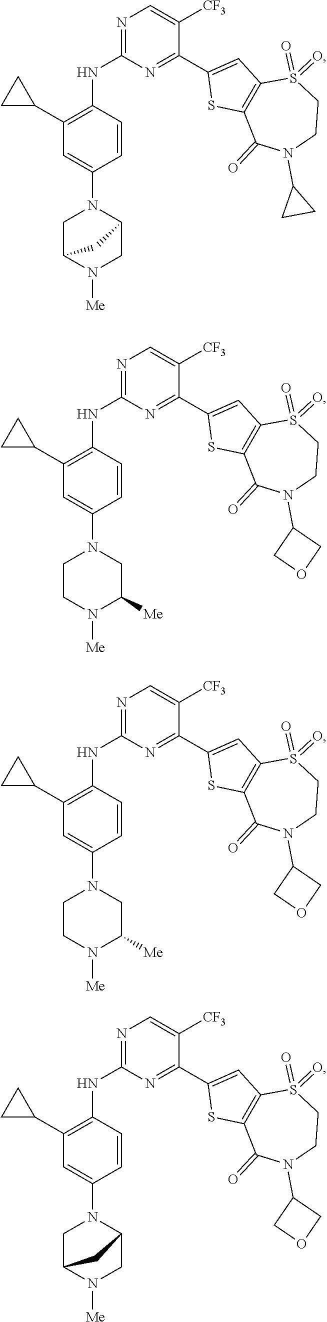 [SMILES: CN1C[C@H]2C[C@@H]1CN2c1ccc(Nc2ncc(C(F)(F)F)c(-c3cc4c(s3)C(=O)N(C3CC3)CCS4(=O)=O)n2)c(C2CC2)c1 ; C[C@@H]1CN(c2ccc(Nc3ncc(C(F)(F)F)c(-c4cc5c(s4)C(=O)N(C4COC4)CCS5(=O)=O)n3)c(C3CC3)c2)CCN1C ; C[C@H]1CN(c2ccc(Nc3ncc(C(F)(F)F)c(-c4cc5c(s4)C(=O)N(C4COC4)CCS5(=O)=O)n3)c(C3CC3)c2)CCN1C ; CN1C[C@@H]2C[C@H]1CN2c1ccc(Nc2ncc(C(F)(F)F)c(-c3cc4c(s3)C(=O)N(C3COC3)CCS4(=O)=O)n2)c(C2CC2)c1]

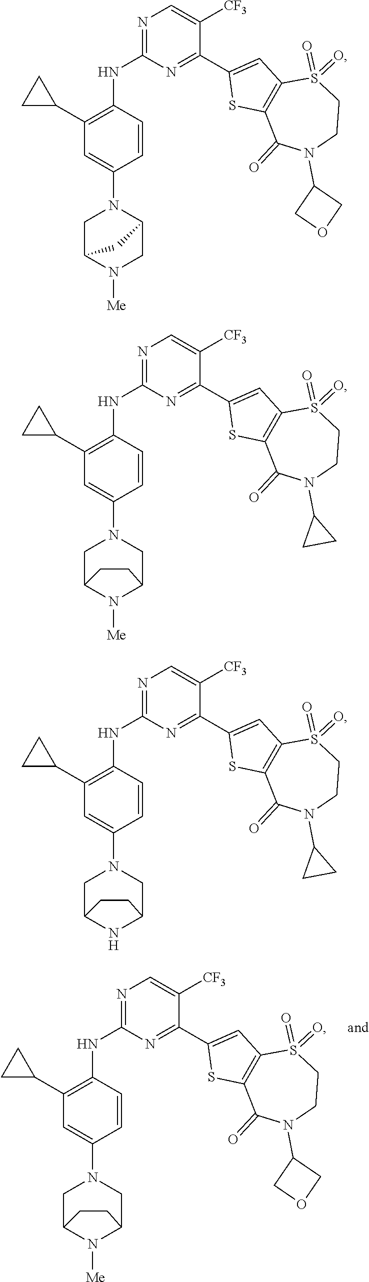 [SMILES: CN1C[C@H]2C[C@@H]1CN2c1ccc(Nc2ncc(C(F)(F)F)c(-c3cc4c(s3)C(=O)N(C3COC3)CCS4(=O)=O)n2)c(C2CC2)c1 ; CN1C2CCC1CN(c1ccc(Nc3ncc(C(F)(F)F)c(-c4cc5c(s4)C(=O)N(C4CC4)CCS5(=O)=O)n3)c(C3CC3)c1)C2 ; O=C1c2sc(-c3nc(Nc4ccc(N5CC6CCC(C5)N6)cc4C4CC4)ncc3C(F)(F)F)cc2S(=O)(=O)CCN1C1CC1 ; CN1C2CCC1CN(c1ccc(Nc3ncc(C(F)(F)F)c(-c4cc5c(s4)C(=O)N(C4COC4)CCS5(=O)=O)n3)c(C3CC3)c1)C2 ; "and"]

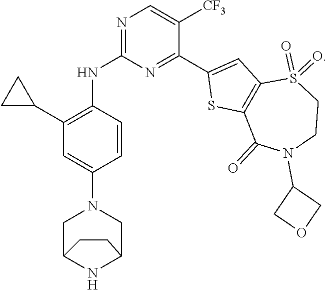 [SMILES: O=C1c2sc(-c3nc(Nc4ccc(N5CC6CCC(C5)N6)cc4C4CC4)ncc3C(F)(F)F)cc2S(=O)(=O)CCN1C1COC1]

In some embodiments, the compound, or a pharmaceutically acceptable salt thereof, is selected from:

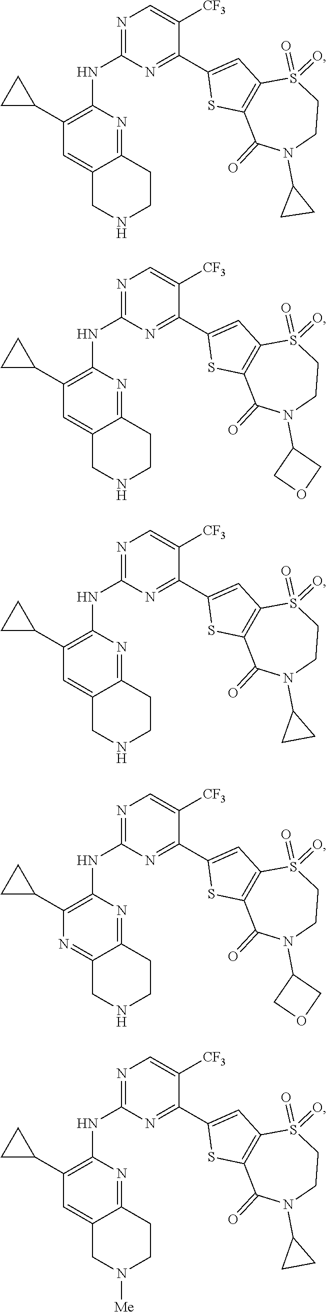 [SMILES: O=C1c2sc(-c3nc(Nc4nc5c(cc4C4CC4)CNCC5)ncc3C(F)(F)F)cc2S(=O)(=O)CCN1C1CC1 ; O=C1c2sc(-c3nc(Nc4nc5c(cc4C4CC4)CNCC5)ncc3C(F)(F)F)cc2S(=O)(=O)CCN1C1COC1 ; O=C1c2sc(-c3nc(Nc4nc5c(cc4C4CC4)CNCC5)ncc3C(F)(F)F)cc2S(=O)(=O)CCN1C1CC1 ; O=C1c2sc(-c3nc(Nc4nc5c(nc4C4CC4)CNCC5)ncc3C(F)(F)F)cc2S(=O)(=O)CCN1C1COC1 ; CN1CCc2nc(Nc3ncc(C(F)(F)F)c(-c4cc5c(s4)C(=O)N(C4CC4)CCS5(=O)=O)n3)c(C3CC3)cc2C1]

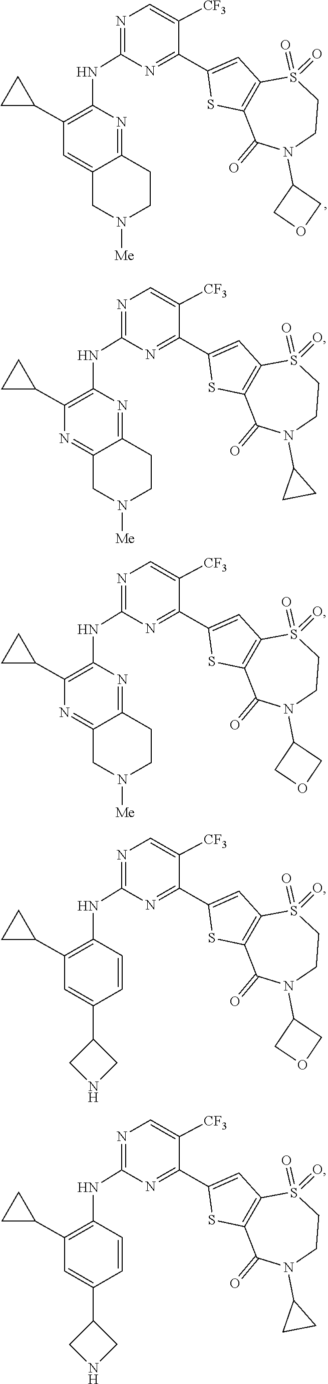

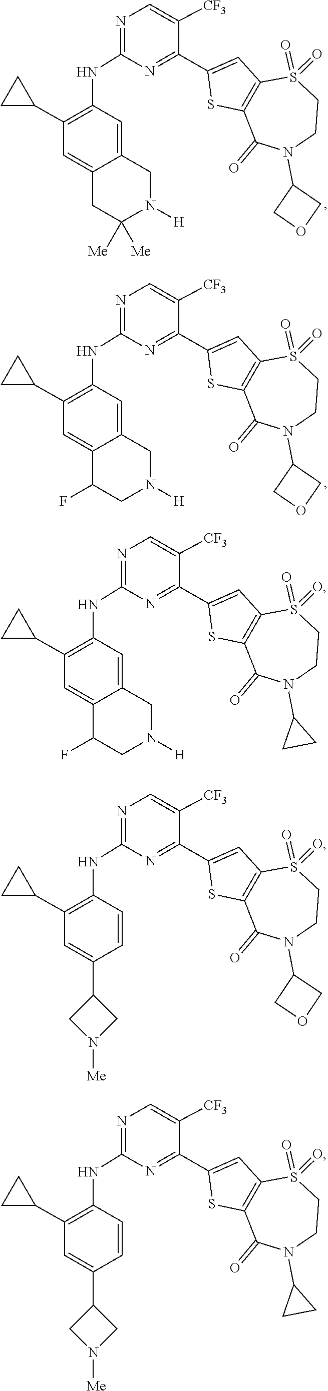

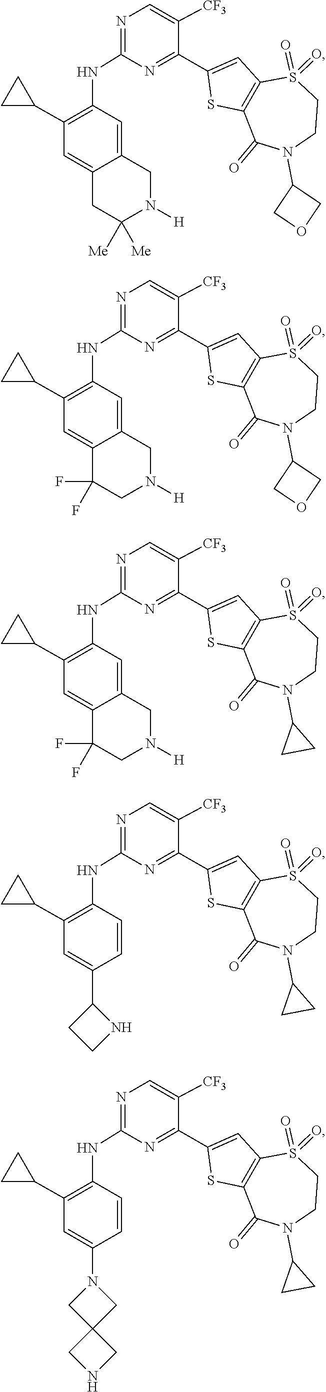

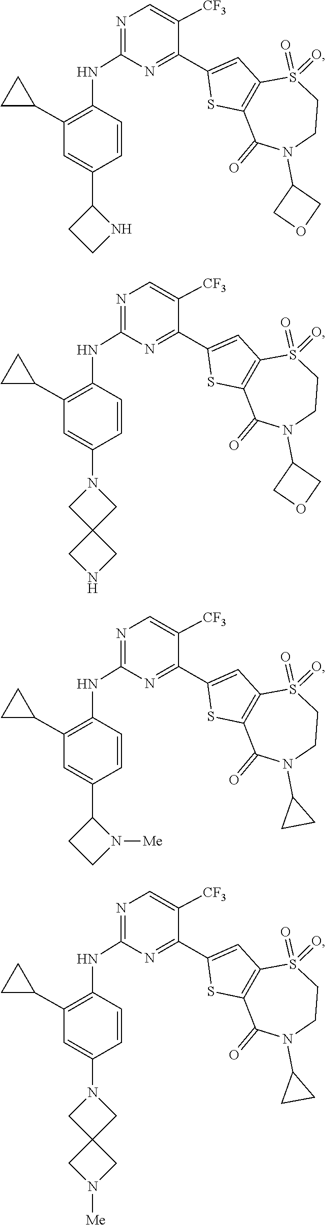 [SMILES: O=C1c2sc(-c3nc(Nc4ccc(C5CCN5)cc4C4CC4)ncc3C(F)(F)F)cc2S(=O)(=O)CCN1C1COC1 ; O=C1c2sc(-c3nc(Nc4ccc(N5CC6(CNC6)C5)cc4C4CC4)ncc3C(F)(F)F)cc2S(=O)(=O)CCN1C1COC1 ; CN1CCC1c1ccc(Nc2ncc(C(F)(F)F)c(-c3cc4c(s3)C(=O)N(C3CC3)CCS4(=O)=O)n2)c(C2CC2)c1 ; CN1CC2(C1)CN(c1ccc(Nc3ncc(C(F)(F)F)c(-c4cc5c(s4)C(=O)N(C4CC4)CCS5(=O)=O)n3)c(C3CC3)c1)C2]

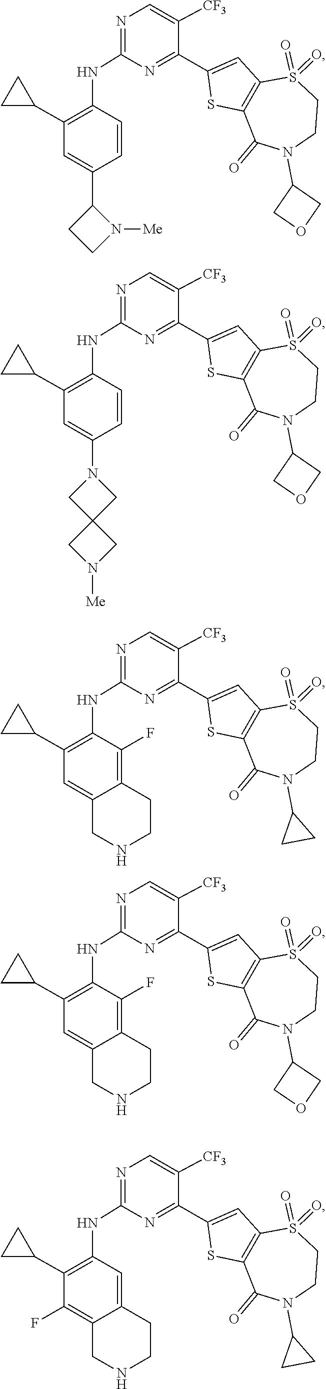

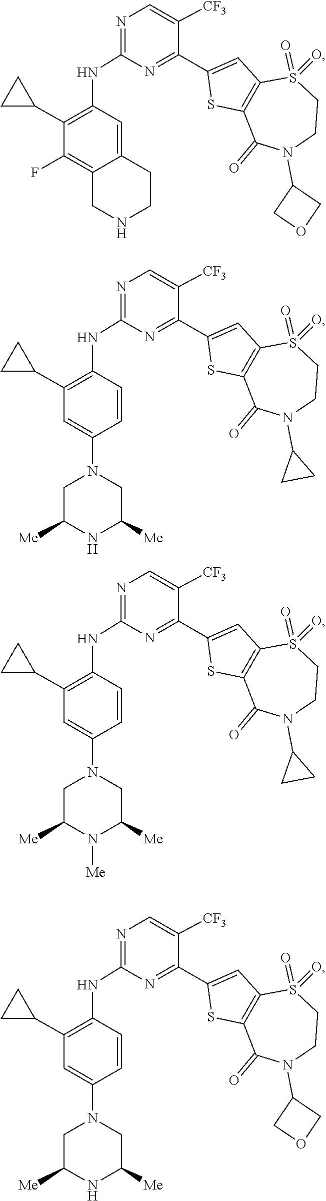

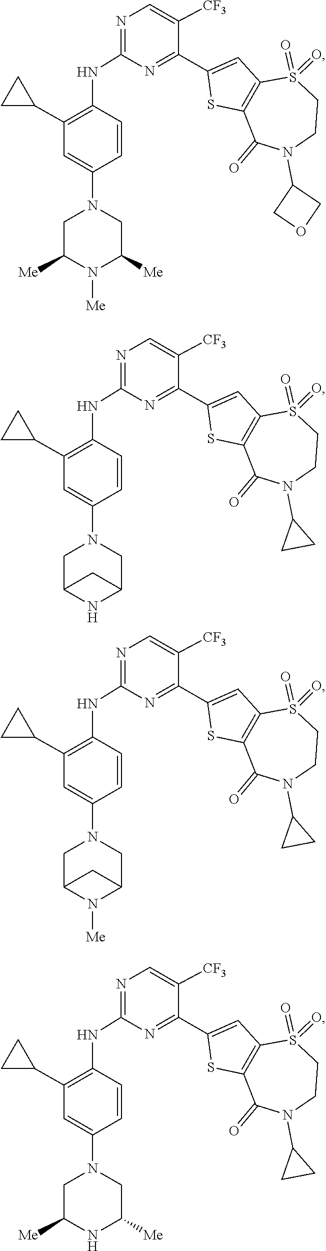 [SMILES: C[C@@H]1CN(c2ccc(Nc3ncc(C(F)(F)F)c(-c4cc5c(s4)C(=O)N(C4COC4)CCS5(=O)=O)n3)c(C3CC3)c2)C[C@H](C)N1C ; O=C1c2sc(-c3nc(Nc4ccc(N5CC6CC(C5)N6)cc4C4CC4)ncc3C(F)(F)F)cc2S(=O)(=O)CCN1C1CC1 ; CN1C2CC1CN(c1ccc(Nc3ncc(C(F)(F)F)c(-c4cc5c(s4)C(=O)N(C4CC4)CCS5(=O)=O)n3)c(C3CC3)c1)C2 ; C[C@H]1CN(c2ccc(Nc3ncc(C(F)(F)F)c(-c4cc5c(s4)C(=O)N(C4CC4)CCS5(=O)=O)n3)c(C3CC3)c2)C[C@H](C)N1]

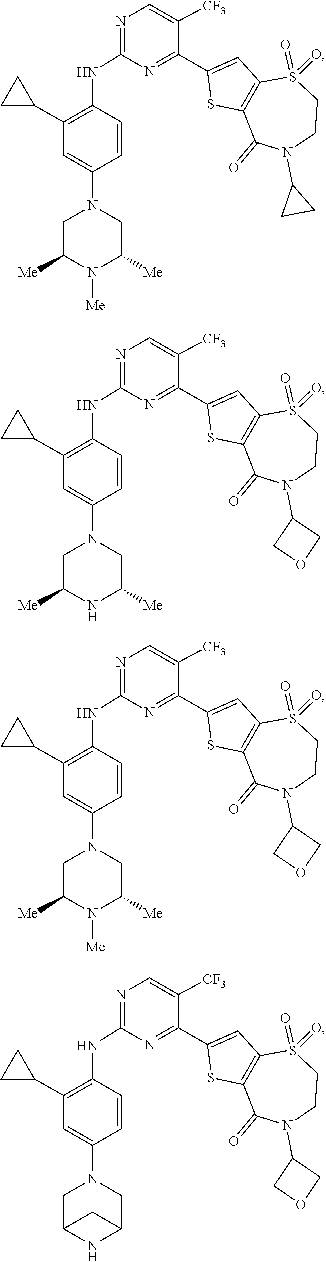

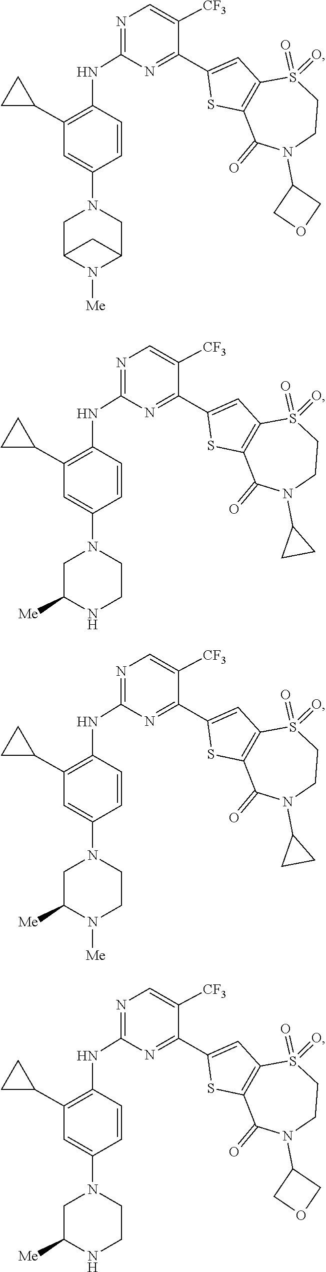 [SMILES: CN1C2CC1CN(c1ccc(Nc3ncc(C(F)(F)F)c(-c4cc5c(s4)C(=O)N(C4COC4)CCS5(=O)=O)n3)c(C3CC3)c1)C2 ; C[C@H]1CN(c2ccc(Nc3ncc(C(F)(F)F)c(-c4cc5c(s4)C(=O)N(C4CC4)CCS5(=O)=O)n3)c(C3CC3)c2)CCN1 ; C[C@H]1CN(c2ccc(Nc3ncc(C(F)(F)F)c(-c4cc5c(s4)C(=O)N(C4CC4)CCS5(=O)=O)n3)c(C3CC3)c2)CCN1C ; C[C@H]1CN(c2ccc(Nc3ncc(C(F)(F)F)c(-c4cc5c(s4)C(=O)N(C4COC4)CCS5(=O)=O)n3)c(C3CC3)c2)CCN1]

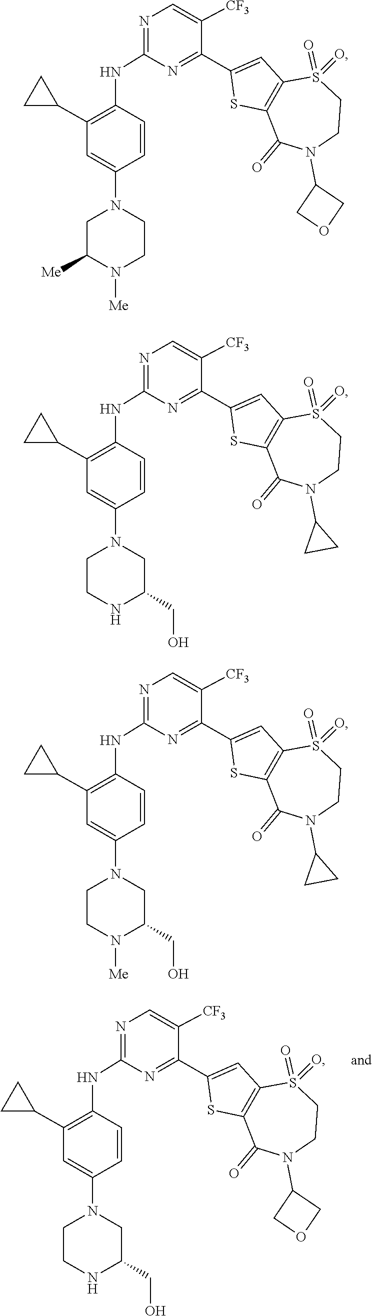

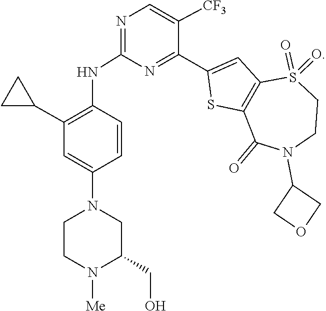 [SMILES: CN1CCN(c2ccc(Nc3ncc(C(F)(F)F)c(-c4cc5c(s4)C(=O)N(C4COC4)CCS5(=O)=O)n3)c(C3CC3)c2)C[C@@H]1CO]

In some embodiments, the compound, or a pharmaceutically acceptable salt thereof, is selected from:

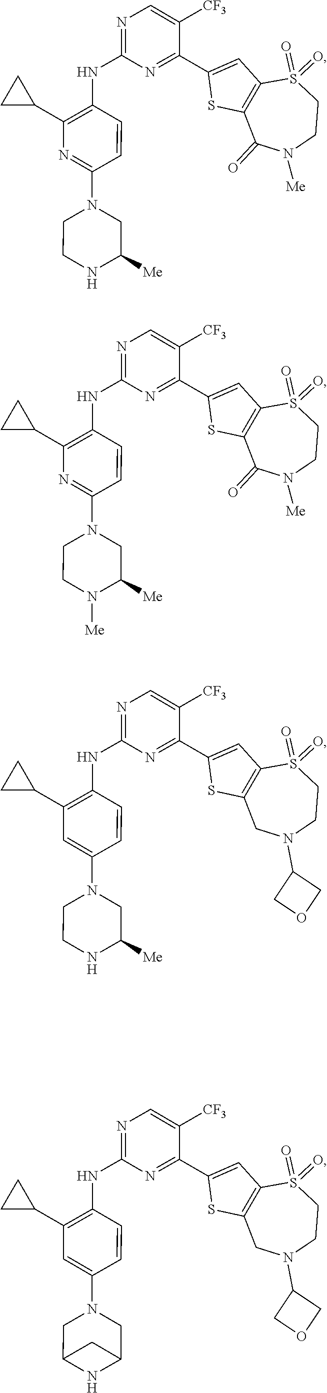 [SMILES: C[C@@H]1CN(c2ccc(Nc3ncc(C(F)(F)F)c(-c4cc5c(s4)C(=O)N(C)CCS5(=O)=O)n3)c(C3CC3)n2)CCN1 ; C[C@@H]1CN(c2ccc(Nc3ncc(C(F)(F)F)c(-c4cc5c(s4)C(=O)N(C)CCS5(=O)=O)n3)c(C3CC3)n2)CCN1C ; C[C@@H]1CN(c2ccc(Nc3ncc(C(F)(F)F)c(-c4cc5c(s4)CN(C4COC4)CCS5(=O)=O)n3)c(C3CC3)c2)CCN1 ; O=S1(=O)CCN(C2COC2)Cc2sc(-c3nc(Nc4ccc(N5CC6CC(C5)N6)cc4C4CC4)ncc3C(F)(F)F)cc21]

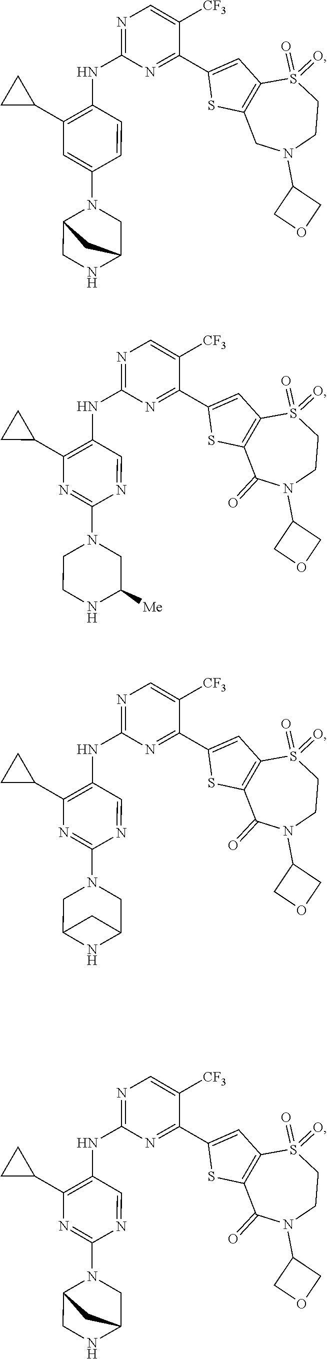 [SMILES: O=S1(=O)CCN(C2COC2)Cc2sc(-c3nc(Nc4ccc(N5C[C@H]6C[C@@H]5CN6)cc4C4CC4)ncc3C(F)(F)F)cc21 ; C[C@@H]1CN(c2ncc(Nc3ncc(C(F)(F)F)c(-c4cc5c(s4)C(=O)N(C4COC4)CCS5(=O)=O)n3)c(C3CC3)n2)CCN1 ; O=C1c2sc(-c3nc(Nc4cnc(N5CC6CC(C5)N6)nc4C4CC4)ncc3C(F)(F)F)cc2S(=O)(=O)CCN1C1COC1 ; O=C1c2sc(-c3nc(Nc4cnc(N5C[C@H]6C[C@@H]5CN6)nc4C4CC4)ncc3C(F)(F)F)cc2S(=O)(=O)CCN1C1COC1]

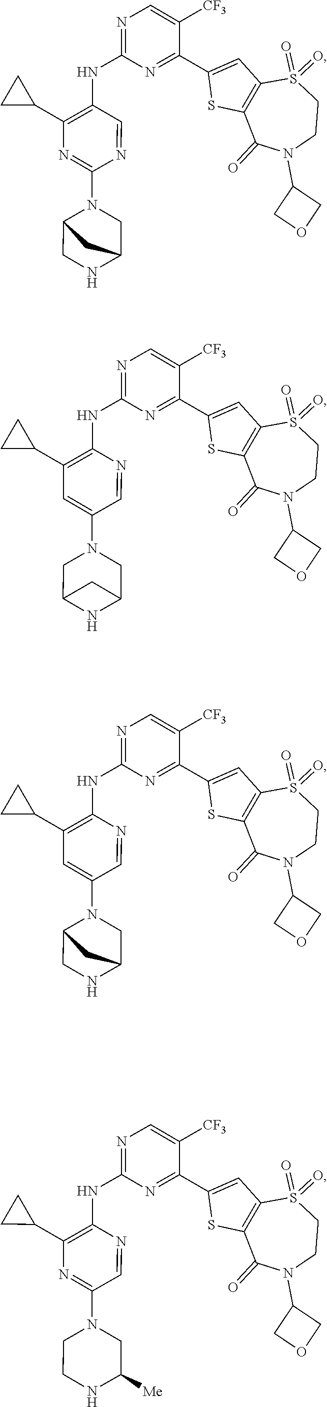 [SMILES: O=C1c2sc(-c3nc(Nc4cnc(N5C[C@H]6C[C@@H]5CN6)nc4C4CC4)ncc3C(F)(F)F)cc2S(=O)(=O)CCN1C1COC1 ; O=C1c2sc(-c3nc(Nc4ncc(N5CC6CC(C5)N6)cc4C4CC4)ncc3C(F)(F)F)cc2S(=O)(=O)CCN1C1COC1 ; O=C1c2sc(-c3nc(Nc4ncc(N5C[C@H]6C[C@@H]5CN6)cc4C4CC4)ncc3C(F)(F)F)cc2S(=O)(=O)CCN1C1COC1 ; C[C@@H]1CN(c2cnc(Nc3ncc(C(F)(F)F)c(-c4cc5c(s4)C(=O)N(C4COC4)CCS5(=O)=O)n3)c(C3CC3)n2)CCN1]

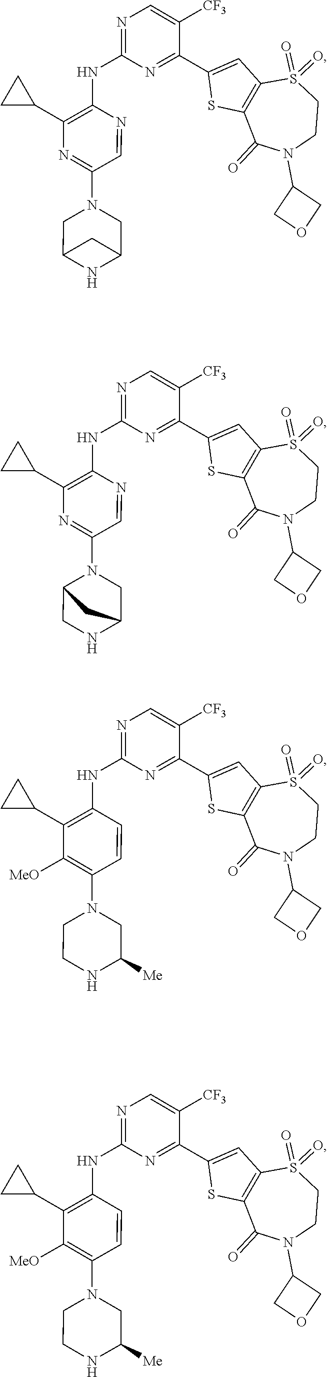 [SMILES: O=C1c2sc(-c3nc(Nc4ncc(N5CC6CC(C5)N6)nc4C4CC4)ncc3C(F)(F)F)cc2S(=O)(=O)CCN1C1COC1 ; O=C1c2sc(-c3nc(Nc4ncc(N5C[C@H]6C[C@@H]5CN6)nc4C4CC4)ncc3C(F)(F)F)cc2S(=O)(=O)CCN1C1COC1 ; COc1c(N2CCN[C@H](C)C2)ccc(Nc2ncc(C(F)(F)F)c(-c3cc4c(s3)C(=O)N(C3COC3)CCS4(=O)=O)n2)c1C1CC1 ; COc1c(N2CCN[C@H](C)C2)ccc(Nc2ncc(C(F)(F)F)c(-c3cc4c(s3)C(=O)N(C3COC3)CCS4(=O)=O)n2)c1C1CC1]

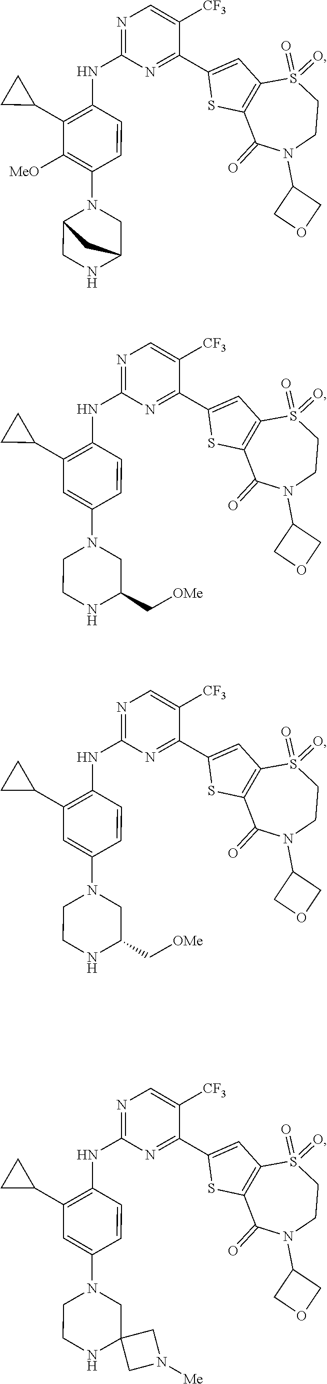 [SMILES: COc1c(N2C[C@H]3C[C@@H]2CN3)ccc(Nc2ncc(C(F)(F)F)c(-c3cc4c(s3)C(=O)N(C3COC3)CCS4(=O)=O)n2)c1C1CC1 ; COC[C@@H]1CN(c2ccc(Nc3ncc(C(F)(F)F)c(-c4cc5c(s4)C(=O)N(C4COC4)CCS5(=O)=O)n3)c(C3CC3)c2)CCN1 ; COC[C@H]1CN(c2ccc(Nc3ncc(C(F)(F)F)c(-c4cc5c(s4)C(=O)N(C4COC4)CCS5(=O)=O)n3)c(C3CC3)c2)CCN1 ; CN1CC2(C1)CN(c1ccc(Nc3ncc(C(F)(F)F)c(-c4cc5c(s4)C(=O)N(C4COC4)CCS5(=O)=O)n3)c(C3CC3)c1)CCN2]

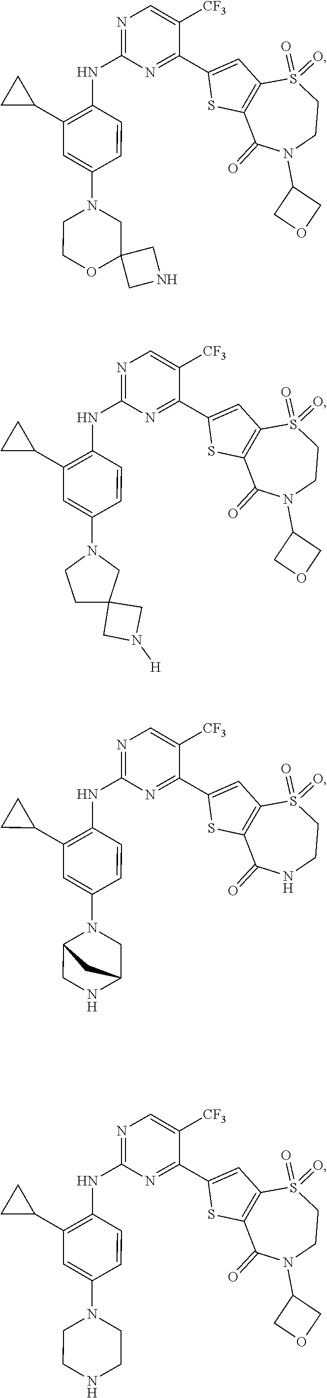 [SMILES: O=C1c2sc(-c3nc(Nc4ccc(N5CCOC6(CNC6)C5)cc4C4CC4)ncc3C(F)(F)F)cc2S(=O)(=O)CCN1C1COC1 ; O=C1c2sc(-c3nc(Nc4ccc(N5CCC6(CNC6)C5)cc4C4CC4)ncc3C(F)(F)F)cc2S(=O)(=O)CCN1C1COC1 ; O=C1NCCS(=O)(=O)c2cc(-c3nc(Nc4ccc(N5C[C@H]6C[C@@H]5CN6)cc4C4CC4)ncc3C(F)(F)F)sc21 ; O=C1c2sc(-c3nc(Nc4ccc(N5CCNCC5)cc4C4CC4)ncc3C(F)(F)F)cc2S(=O)(=O)CCN1C1COC1]

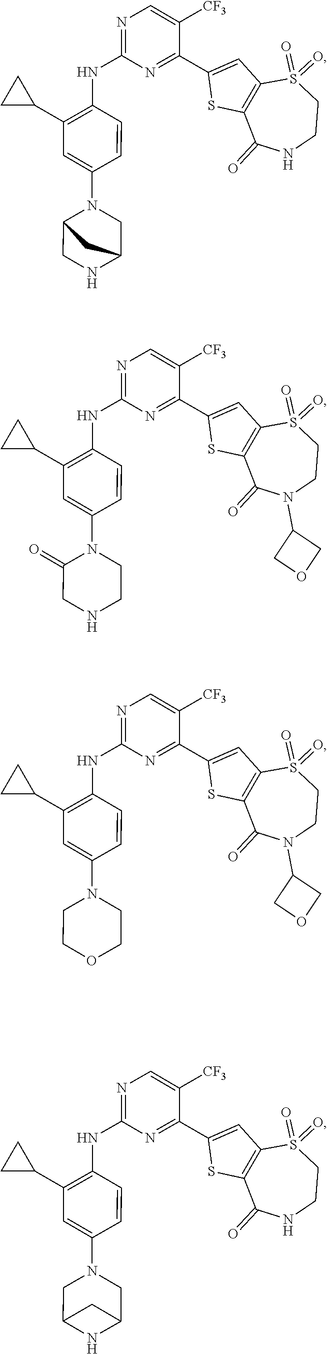 [SMILES: O=C1NCCS(=O)(=O)c2cc(-c3nc(Nc4ccc(N5C[C@H]6C[C@@H]5CN6)cc4C4CC4)ncc3C(F)(F)F)sc21 ; O=C1CNCCN1c1ccc(Nc2ncc(C(F)(F)F)c(-c3cc4c(s3)C(=O)N(C3COC3)CCS4(=O)=O)n2)c(C2CC2)c1 ; O=C1c2sc(-c3nc(Nc4ccc(N5CCOCC5)cc4C4CC4)ncc3C(F)(F)F)cc2S(=O)(=O)CCN1C1COC1 ; O=C1NCCS(=O)(=O)c2cc(-c3nc(Nc4ccc(N5CC6CC(C5)N6)cc4C4CC4)ncc3C(F)(F)F)sc21]

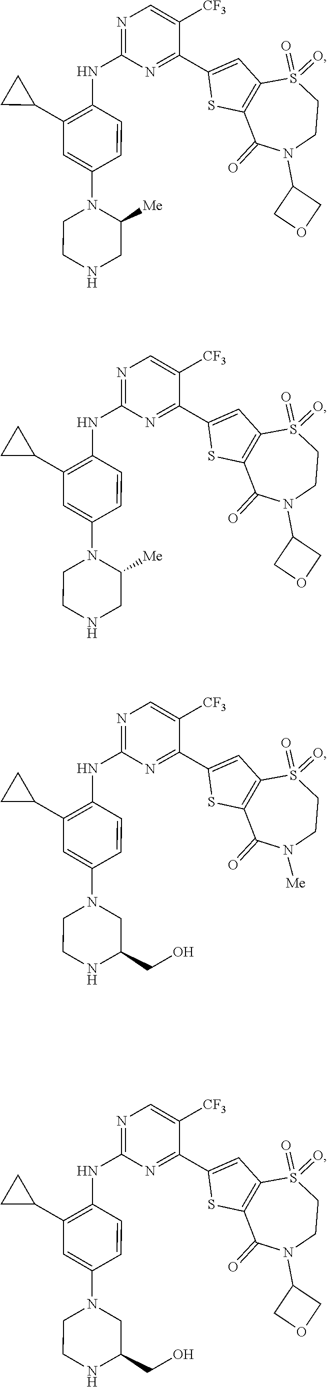 [SMILES: C[C@H]1CNCCN1c1ccc(Nc2ncc(C(F)(F)F)c(-c3cc4c(s3)C(=O)N(C3COC3)CCS4(=O)=O)n2)c(C2CC2)c1 ; C[C@@H]1CNCCN1c1ccc(Nc2ncc(C(F)(F)F)c(-c3cc4c(s3)C(=O)N(C3COC3)CCS4(=O)=O)n2)c(C2CC2)c1 ; CN1CCS(=O)(=O)c2cc(-c3nc(Nc4ccc(N5CCN[C@H](CO)C5)cc4C4CC4)ncc3C(F)(F)F)sc2C1=O ; O=C1c2sc(-c3nc(Nc4ccc(N5CCN[C@H](CO)C5)cc4C4CC4)ncc3C(F)(F)F)cc2S(=O)(=O)CCN1C1COC1]

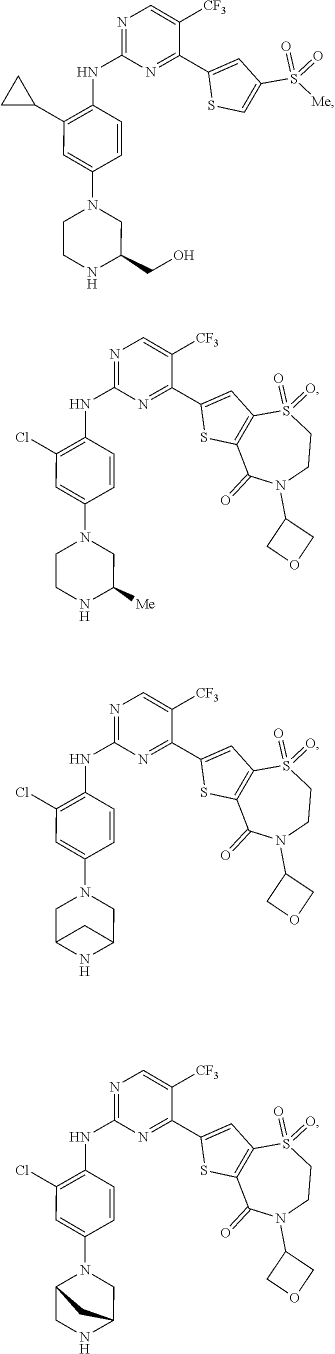 [SMILES: CS(=O)(=O)c1csc(-c2nc(Nc3ccc(N4CCN[C@H](CO)C4)cc3C3CC3)ncc2C(F)(F)F)c1 ; C[C@@H]1CN(c2ccc(Nc3ncc(C(F)(F)F)c(-c4cc5c(s4)C(=O)N(C4COC4)CCS5(=O)=O)n3)c(Cl)c2)CCN1 ; O=C1c2sc(-c3nc(Nc4ccc(N5CC6CC(C5)N6)cc4Cl)ncc3C(F)(F)F)cc2S(=O)(=O)CCN1C1COC1 ; O=C1c2sc(-c3nc(Nc4ccc(N5C[C@H]6C[C@@H]5CN6)cc4Cl)ncc3C(F)(F)F)cc2S(=O)(=O)CCN1C1COC1]

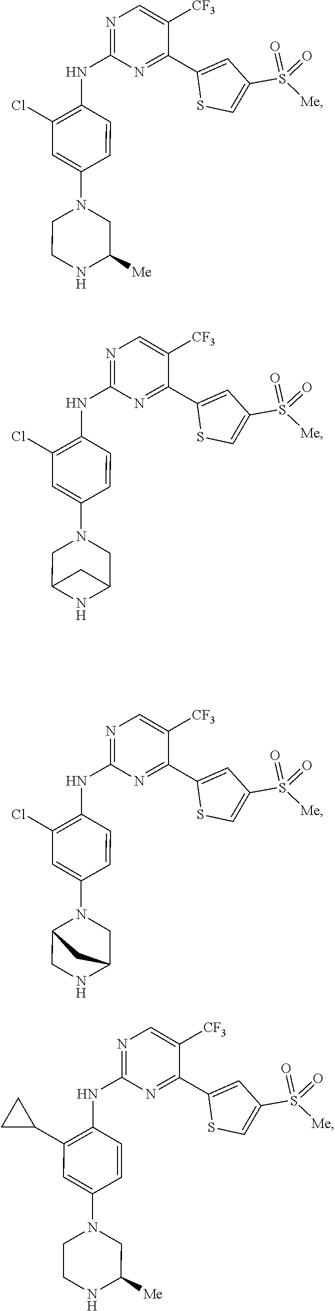 [SMILES: C[C@@H]1CN(c2ccc(Nc3ncc(C(F)(F)F)c(-c4cc(S(C)(=O)=O)cs4)n3)c(Cl)c2)CCN1 ; CS(=O)(=O)c1csc(-c2nc(Nc3ccc(N4CC5CC(C4)N5)cc3Cl)ncc2C(F)(F)F)c1 ; CS(=O)(=O)c1csc(-c2nc(Nc3ccc(N4C[C@H]5C[C@@H]4CN5)cc3Cl)ncc2C(F)(F)F)c1 ; C[C@@H]1CN(c2ccc(Nc3ncc(C(F)(F)F)c(-c4cc(S(C)(=O)=O)cs4)n3)c(C3CC3)c2)CCN1]

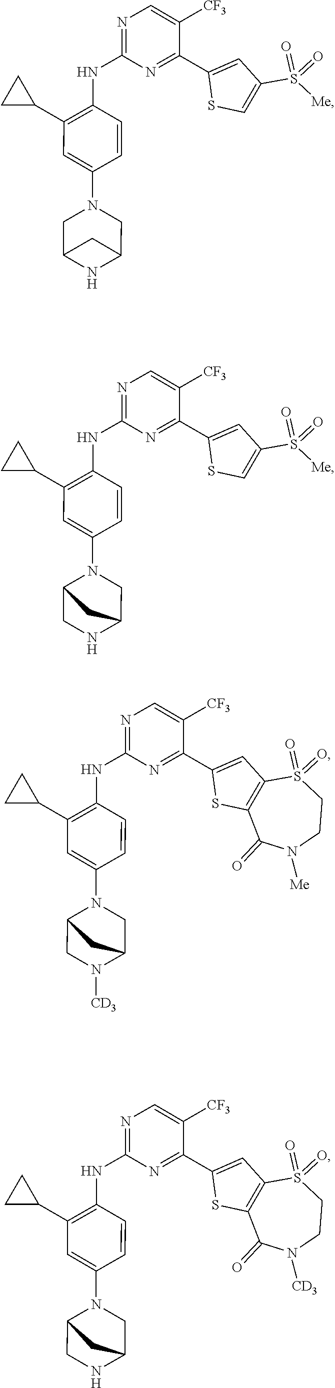 [SMILES: CS(=O)(=O)c1csc(-c2nc(Nc3ccc(N4CC5CC(C4)N5)cc3C3CC3)ncc2C(F)(F)F)c1 ; CS(=O)(=O)c1csc(-c2nc(Nc3ccc(N4C[C@H]5C[C@@H]4CN5)cc3C3CC3)ncc2C(F)(F)F)c1 ; [2H]C([2H])([2H])N1C[C@H]2C[C@@H]1CN2c1ccc(Nc2ncc(C(F)(F)F)c(-c3cc4c(s3)C(=O)N(C)CCS4(=O)=O)n2)c(C2CC2)c1 ; [2H]C([2H])([2H])N1CCS(=O)(=O)c2cc(-c3nc(Nc4ccc(N5C[C@H]6C[C@@H]5CN6)cc4C4CC4)ncc3C(F)(F)F)sc2C1=O]

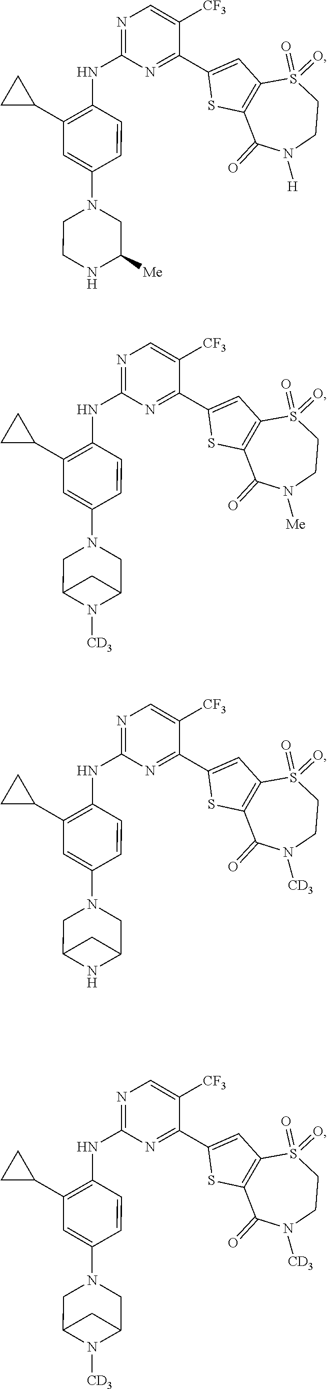 [SMILES: C[C@@H]1CN(c2ccc(Nc3ncc(C(F)(F)F)c(-c4cc5c(s4)C(=O)NCCS5(=O)=O)n3)c(C3CC3)c2)CCN1 ; [2H]C([2H])([2H])N1C2CC1CN(c1ccc(Nc3ncc(C(F)(F)F)c(-c4cc5c(s4)C(=O)N(C)CCS5(=O)=O)n3)c(C3CC3)c1)C2 ; [2H]C([2H])([2H])N1CCS(=O)(=O)c2cc(-c3nc(Nc4ccc(N5CC6CC(C5)N6)cc4C4CC4)ncc3C(F)(F)F)sc2C1=O ; [2H]C([2H])([2H])N1CCS(=O)(=O)c2cc(-c3nc(Nc4ccc(N5CC6CC(C5)N6C([2H])([2H])[2H])cc4C4CC4)ncc3C(F)(F)F)sc2C1=O]

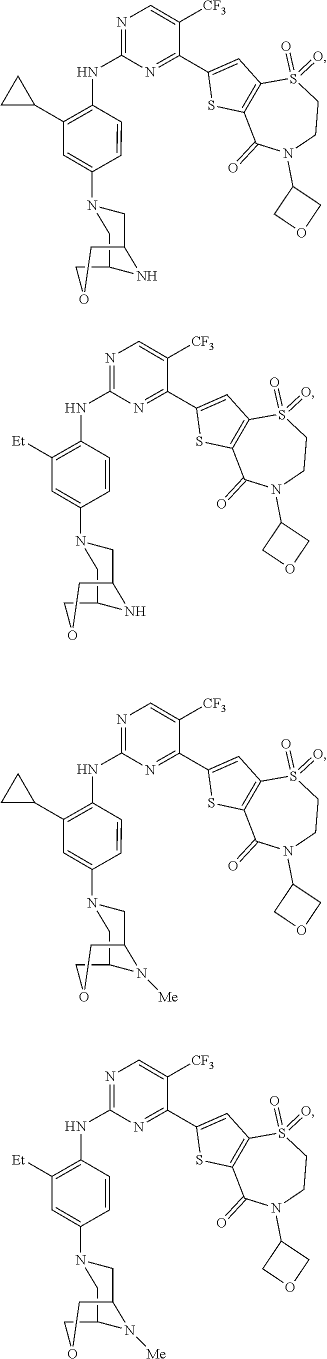 [SMILES: O=C1c2sc(-c3nc(Nc4ccc(N5CC6COCC(C5)N6)cc4C4CC4)ncc3C(F)(F)F)cc2S(=O)(=O)CCN1C1COC1 ; CCc1cc(N2CC3COCC(C2)N3)ccc1Nc1ncc(C(F)(F)F)c(-c2cc3c(s2)C(=O)N(C2COC2)CCS3(=O)=O)n1 ; CN1C2COCC1CN(c1ccc(Nc3ncc(C(F)(F)F)c(-c4cc5c(s4)C(=O)N(C4COC4)CCS5(=O)=O)n3)c(C3CC3)c1)C2 ; CCc1cc(N2CC3COCC(C2)N3C)ccc1Nc1ncc(C(F)(F)F)c(-c2cc3c(s2)C(=O)N(C2COC2)CCS3(=O)=O)n1]

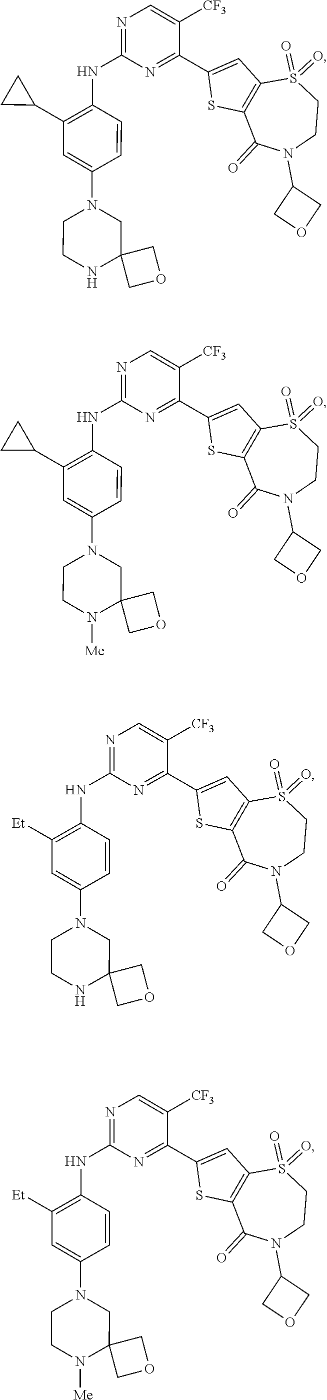 [SMILES: O=C1c2sc(-c3nc(Nc4ccc(N5CCNC6(COC6)C5)cc4C4CC4)ncc3C(F)(F)F)cc2S(=O)(=O)CCN1C1COC1 ; CN1CCN(c2ccc(Nc3ncc(C(F)(F)F)c(-c4cc5c(s4)C(=O)N(C4COC4)CCS5(=O)=O)n3)c(C3CC3)c2)CC12COC2 ; CCc1cc(N2CCNC3(COC3)C2)ccc1Nc1ncc(C(F)(F)F)c(-c2cc3c(s2)C(=O)N(C2COC2)CCS3(=O)=O)n1 ; CCc1cc(N2CCN(C)C3(COC3)C2)ccc1Nc1ncc(C(F)(F)F)c(-c2cc3c(s2)C(=O)N(C2COC2)CCS3(=O)=O)n1]

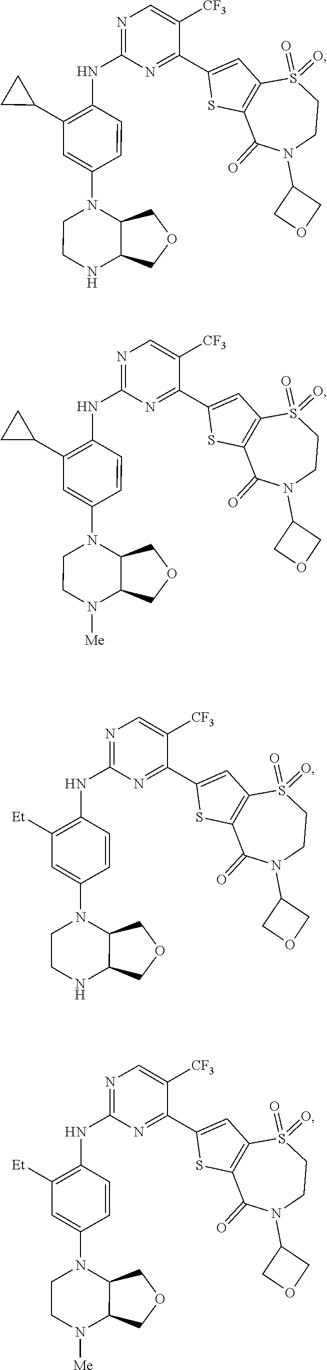 [SMILES: O=C1c2sc(-c3nc(Nc4ccc(N5CCN[C@@H]6COC[C@@H]65)cc4C4CC4)ncc3C(F)(F)F)cc2S(=O)(=O)CCN1C1COC1 ; CN1CCN(c2ccc(Nc3ncc(C(F)(F)F)c(-c4cc5c(s4)C(=O)N(C4COC4)CCS5(=O)=O)n3)c(C3CC3)c2)[C@H]2COC[C@H]21 ; CCc1cc(N2CCN[C@@H]3COC[C@@H]32)ccc1Nc1ncc(C(F)(F)F)c(-c2cc3c(s2)C(=O)N(C2COC2)CCS3(=O)=O)n1 ; CCc1cc(N2CCN(C)[C@@H]3COC[C@@H]32)ccc1Nc1ncc(C(F)(F)F)c(-c2cc3c(s2)C(=O)N(C2COC2)CCS3(=O)=O)n1]

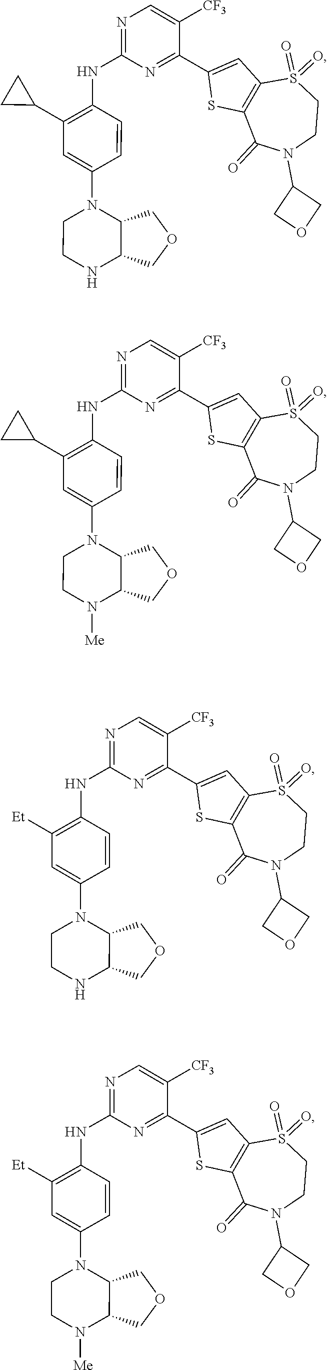 [SMILES: O=C1c2sc(-c3nc(Nc4ccc(N5CCN[C@H]6COC[C@H]65)cc4C4CC4)ncc3C(F)(F)F)cc2S(=O)(=O)CCN1C1COC1 ; CN1CCN(c2ccc(Nc3ncc(C(F)(F)F)c(-c4cc5c(s4)C(=O)N(C4COC4)CCS5(=O)=O)n3)c(C3CC3)c2)[C@@H]2COC[C@@H]21 ; CCc1cc(N2CCN[C@H]3COC[C@H]32)ccc1Nc1ncc(C(F)(F)F)c(-c2cc3c(s2)C(=O)N(C2COC2)CCS3(=O)=O)n1 ; CCc1cc(N2CCN(C)[C@H]3COC[C@H]32)ccc1Nc1ncc(C(F)(F)F)c(-c2cc3c(s2)C(=O)N(C2COC2)CCS3(=O)=O)n1]

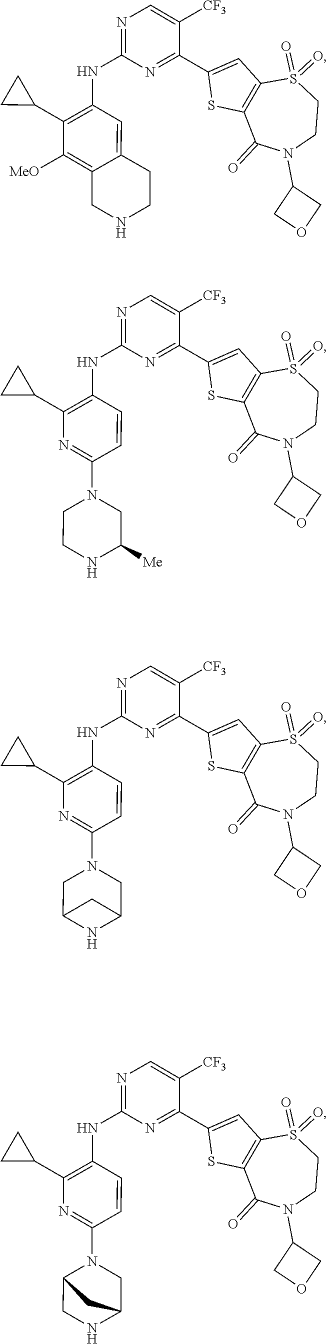 [SMILES: COc1c2c(cc(Nc3ncc(C(F)(F)F)c(-c4cc5c(s4)C(=O)N(C4COC4)CCS5(=O)=O)n3)c1C1CC1)CCNC2 ; C[C@@H]1CN(c2ccc(Nc3ncc(C(F)(F)F)c(-c4cc5c(s4)C(=O)N(C4COC4)CCS5(=O)=O)n3)c(C3CC3)n2)CCN1 ; O=C1c2sc(-c3nc(Nc4ccc(N5CC6CC(C5)N6)nc4C4CC4)ncc3C(F)(F)F)cc2S(=O)(=O)CCN1C1COC1 ; O=C1c2sc(-c3nc(Nc4ccc(N5C[C@H]6C[C@@H]5CN6)nc4C4CC4)ncc3C(F)(F)F)cc2S(=O)(=O)CCN1C1COC1]

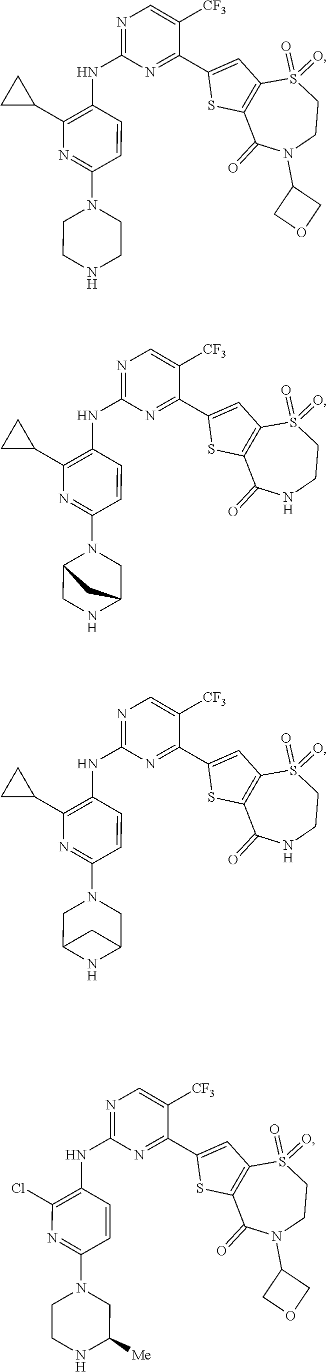 [SMILES: O=C1c2sc(-c3nc(Nc4ccc(N5CCNCC5)nc4C4CC4)ncc3C(F)(F)F)cc2S(=O)(=O)CCN1C1COC1 ; O=C1NCCS(=O)(=O)c2cc(-c3nc(Nc4ccc(N5C[C@H]6C[C@@H]5CN6)nc4C4CC4)ncc3C(F)(F)F)sc21 ; O=C1NCCS(=O)(=O)c2cc(-c3nc(Nc4ccc(N5CC6CC(C5)N6)nc4C4CC4)ncc3C(F)(F)F)sc21 ; C[C@@H]1CN(c2ccc(Nc3ncc(C(F)(F)F)c(-c4cc5c(s4)C(=O)N(C4COC4)CCS5(=O)=O)n3)c(Cl)n2)CCN1]

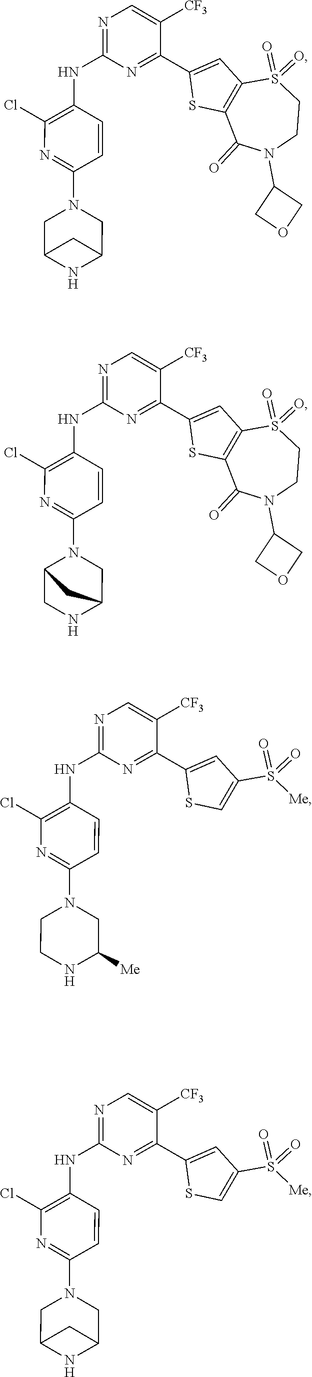 [SMILES: O=C1c2sc(-c3nc(Nc4ccc(N5CC6CC(C5)N6)nc4Cl)ncc3C(F)(F)F)cc2S(=O)(=O)CCN1C1COC1 ; O=C1c2sc(-c3nc(Nc4ccc(N5C[C@H]6C[C@@H]5CN6)nc4Cl)ncc3C(F)(F)F)cc2S(=O)(=O)CCN1C1COC1 ; C[C@@H]1CN(c2ccc(Nc3ncc(C(F)(F)F)c(-c4cc(S(C)(=O)=O)cs4)n3)c(Cl)n2)CCN1 ; CS(=O)(=O)c1csc(-c2nc(Nc3ccc(N4CC5CC(C4)N5)nc3Cl)ncc2C(F)(F)F)c1]

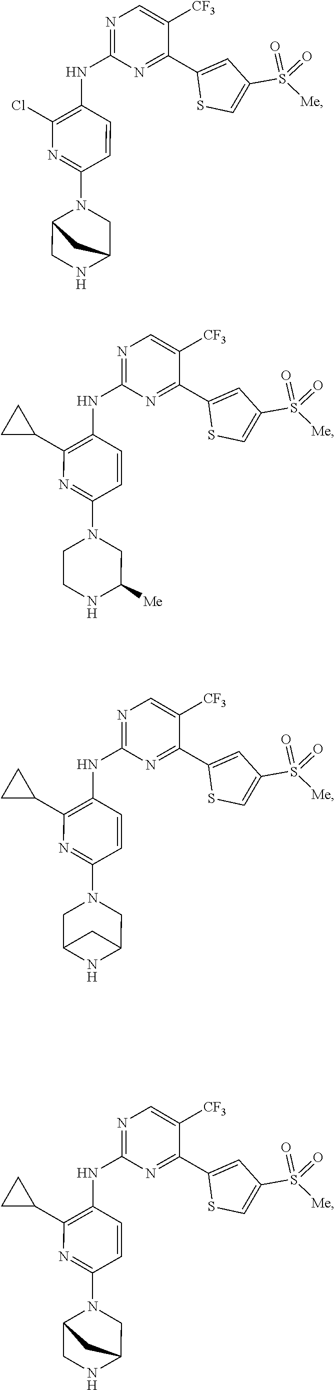 [SMILES: CS(=O)(=O)c1csc(-c2nc(Nc3ccc(N4C[C@H]5C[C@@H]4CN5)nc3Cl)ncc2C(F)(F)F)c1 ; C[C@@H]1CN(c2ccc(Nc3ncc(C(F)(F)F)c(-c4cc(S(C)(=O)=O)cs4)n3)c(C3CC3)n2)CCN1 ; CS(=O)(=O)c1csc(-c2nc(Nc3ccc(N4CC5CC(C4)N5)nc3C3CC3)ncc2C(F)(F)F)c1 ; CS(=O)(=O)c1csc(-c2nc(Nc3ccc(N4C[C@H]5C[C@@H]4CN5)nc3C3CC3)ncc2C(F)(F)F)c1]

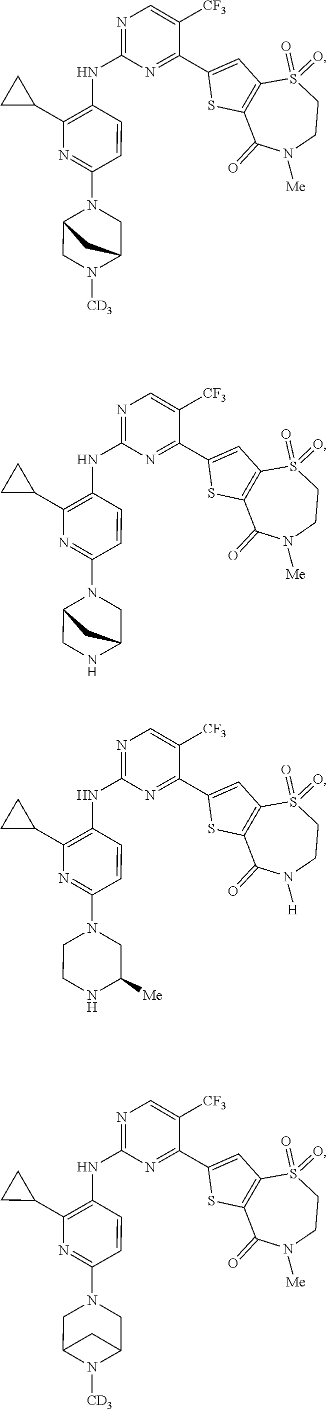 [SMILES: [2H]C([2H])([2H])N1C[C@H]2C[C@@H]1CN2c1ccc(Nc2ncc(C(F)(F)F)c(-c3cc4c(s3)C(=O)N(C)CCS4(=O)=O)n2)c(C2CC2)n1 ; CN1CCS(=O)(=O)c2cc(-c3nc(Nc4ccc(N5C[C@H]6C[C@@H]5CN6)nc4C4CC4)ncc3C(F)(F)F)sc2C1=O ; C[C@@H]1CN(c2ccc(Nc3ncc(C(F)(F)F)c(-c4cc5c(s4)C(=O)NCCS5(=O)=O)n3)c(C3CC3)n2)CCN1 ; [2H]C([2H])([2H])N1C2CC1CN(c1ccc(Nc3ncc(C(F)(F)F)c(-c4cc5c(s4)C(=O)N(C)CCS5(=O)=O)n3)c(C3CC3)n1)C2]

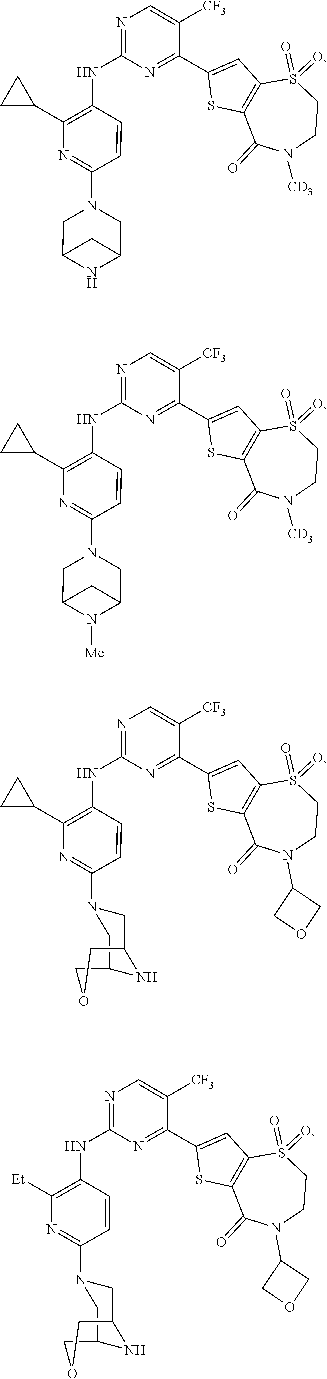 [SMILES: [2H]C([2H])([2H])N1CCS(=O)(=O)c2cc(-c3nc(Nc4ccc(N5CC6CC(C5)N6)nc4C4CC4)ncc3C(F)(F)F)sc2C1=O ; [2H]C([2H])([2H])N1CCS(=O)(=O)c2cc(-c3nc(Nc4ccc(N5CC6CC(C5)N6C)nc4C4CC4)ncc3C(F)(F)F)sc2C1=O ; O=C1c2sc(-c3nc(Nc4ccc(N5CC6COCC(C5)N6)nc4C4CC4)ncc3C(F)(F)F)cc2S(=O)(=O)CCN1C1COC1 ; CCc1nc(N2CC3COCC(C2)N3)ccc1Nc1ncc(C(F)(F)F)c(-c2cc3c(s2)C(=O)N(C2COC2)CCS3(=O)=O)n1]

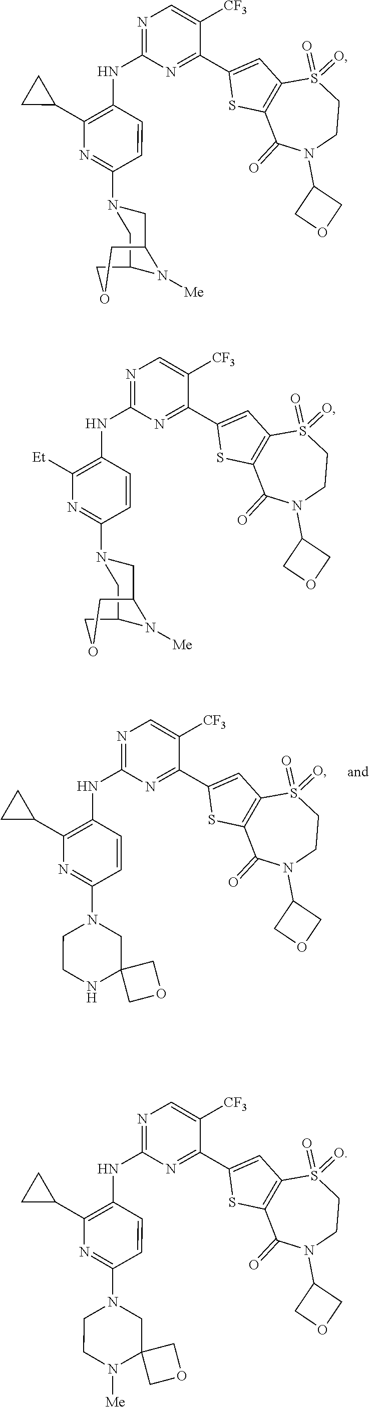 [SMILES: CN1C2COCC1CN(c1ccc(Nc3ncc(C(F)(F)F)c(-c4cc5c(s4)C(=O)N(C4COC4)CCS5(=O)=O)n3)c(C3CC3)n1)C2 ; CCc1nc(N2CC3COCC(C2)N3C)ccc1Nc1ncc(C(F)(F)F)c(-c2cc3c(s2)C(=O)N(C2COC2)CCS3(=O)=O)n1 ; O=C1c2sc(-c3nc(Nc4ccc(N5CCNC6(COC6)C5)nc4C4CC4)ncc3C(F)(F)F)cc2S(=O)(=O)CCN1C1COC1 ; CN1CCN(c2ccc(Nc3ncc(C(F)(F)F)c(-c4cc5c(s4)C(=O)N(C4COC4)CCS5(=O)=O)n3)c(C3CC3)n2)CC12COC2]

### Further Forms of Compounds Disclosed Herein

### Isomers Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements.

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% of a total number of hydrogen and deuterium. In some embodiments, one or more of the substituents disclosed herein comprise deuterium at a percentage higher than the natural abundance of deuterium. In some embodiments, one or more hydrogens are replaced with one or more deuteriums in one or more of the substituents disclosed herein.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein is a method of inhibiting a Unc-51 like autophagy activating kinase (ULK) isoform comprising contacting the ULK isoform with a compound disclosed herein or pharmaceutically acceptable salt thereof. In some embodiments, the method inhibits ULK1 and/or ULK2.

Disclosed herein is a method of treating cancer comprising administering to a subject a compound disclosed herein or pharmaceutically acceptable salt thereof.

Disclosed herein is a method of treating cancer sensitive to ULK1/2 inhibition in a subject in need thereof. Some embodiments of the disclosure include methods for treating abnormal cell growth in a subject comprising administering to the subject a therapeutically effective amount of a compound as described herein or a pharmaceutically acceptable salt thereof. In certain such embodiments, the abnormal cell growth is cancer, and in certain of those embodiments the cancer is lung cancer, pancreatic cancer, skin cancer, including melanoma, cancer of the head or neck, ovarian cancer, rectal cancer, colon cancer, breast cancer, cancer of the thyroid gland, chronic or acute leukaemia, and renal cell carcinoma. Such cancers may be KRAS associated cancers. In some embodiments, the cancer comprises a solid tumor. Of particular interest are cancers such as lung cancer, non-small cell lung cancer, colon cancer, rectal, colorectal, gastric, stomach, oesophageal cancer, salivary gland cancer, pancreatic cancer, including pancreatic ductal adenocarcinoma (PDAC), AML, CML, and ovarian cancer. In some embodiment the method of treating cancer is a method of treating chronic myeloid leukaemia. In some embodiments, the cancer comprises a liquid tumor. In some embodiments, the cancer is chronic myeloid leukaemia.

In some embodiments, one or more compounds disclosed herein are administered to subjects having cancer that comprises one or more alterations in the MAPK pathway, including cancers having alternations in one or more of the RAS, SHP2, RAF, MEK, and ERK pathways. In some embodiments, the cancer in the subject has one or more alterations in the RAS pathway. In some embodiments, the cancer in the subject has one or more alterations in the RAF pathway. In some embodiments, the cancer in the subject has one or more alterations in the MEK pathway. In some embodiments, the cancer in the subject has one or more alterations in the ERK pathway. In some embodiments, one or more compounds disclosed herein are administered to subjects having cancer that is driven by cellular signalling in the MAPK pathway.

In some embodiments, one or more compounds disclosed herein are administered to subjects having cancer that comprises one or more alterations in the PI3K-AKT pathway, including cancers having alternations in one or more of the PI3K, PTEN, and AKT pathways. In some embodiments, the cancer in the subject has one or more alterations in the PI3K pathway. In some embodiments, the cancer in the subject has one or more alterations in the PTEN pathway. In some embodiments, the cancer in the subject has one or more alterations in the AKT pathway.

In some embodiments, one or more compounds disclosed herein are administered to subjects having cancer that comprises one or more alterations in the mTOR pathway.

In some embodiments, the cancer in the subject has one or more alterations in the RAS pathway, including mutations to KRAS, including G12C, G12D, and G12V mutations. KRAS inhibitors that may be used in combination with the compounds disclosed herein include, but are not limited to, one or more of AMG 510, MRTX849, and GDC-6036.

In some embodiments, the cancer in the subject has one or more alterations in the RAF pathway, including mutations to BRAF, including BRAF V600E.

In some embodiments, the cancer in the subject has one or more alterations in the ERK pathway.

In some embodiments, the cancer in the subject has one or more alterations in the MEK pathway.

Within the scope of the present disclosure, beneficial or desired clinical results in a subject to which a compound of the disclosure is administered, alone or in the form of a pharmaceutically acceptable composition, include, but are not limited to, one or more of the following: reducing the proliferation of (or destroying) neoplastic or cancerous cell; inhibiting metastasis or neoplastic cells; shrinking or decreasing the size of a tumor; remission of the cancer; decreasing symptoms resulting from the cancer; increasing the quality of life of those suffering from the cancer; decreasing the dose of other medications required to treat the cancer; delaying the progression of the cancer; curing the cancer; overcoming one or more resistance mechanisms of the cancer; and/or prolonging survival of subjects the cancer. Positive therapeutic effects in cancer can be measured in a number of ways (see, for example, W. A. Weber, Assessing tumor response to therapy, J. Nucl. Med. 50 Suppl. 1:1S-10S (2009). For example, with respect to tumor growth inhibition (T/C), according to the National Cancer Institute (NCI) standards, a T/C less than or equal to 42% is the minimum level of anti-tumor activity. A T/C<10% is considered a high anti-tumor activity level, with T/C (%)=median tumor volume of the treated/median tumor volume of the control.times.100.

In some embodiments, the treatment achieved by treatment as disclosed herein is defined by reference to any of the following: partial response (PR), complete response (CR), overall response (OR), progression free survival (PFS), disease free survival (DFS) and overall survival (OS). PFS, also referred to as “Time to Tumor Progression” indicates the length of time during and after treatment that the cancer does not grow and includes the amount of time subjects have experienced a CR or PR, as well as the amount of time subjects have experienced stable disease (SD). DFS refers to the length of time during and after treatment that the subject remains free of disease. OS refers to a prolongation in life expectancy as compared to naive or untreated subjects or subjects. In some embodiments, response to a combination of the disclosure is any of PR, CR, PFS, DFS, OR, or OS that is assessed using Response Evaluation Criteria in Solid Tumors (RECIST) 1.1 response criteria.

The treatment regimen relating to a compound of the disclosure, or a pharmaceutical composition comprising a compound of the disclosure, that is effective to treat cancer in a subject may vary according to factors such as the disease state, age, and weight of the subject, and the ability of the therapy to elicit an anti-cancer response in the subject. While an embodiment of any of the aspects of the disclosure may not be effective in achieving a positive therapeutic effect in every subject, it should do so in a statistically significant number of subjects as determined by any statistical test known in the art such as the Student's t-test, the chi2-test the U-test according to Mann and Whitney, the Kruskal-Wallis test (H-test), Jonckheere-Terpstrat-testy and the Wilcon on-test.

### Dosing

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of or risk factor for the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage, or the frequency of administration, or both, is reduced, as a function of the symptoms, to a level at which the improved disease, disorder or condition is retained. In certain embodiments, however, the patient requires intermittent or daily treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage, or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₁₀ and the ED₉₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the subject every 12 hours; (v) the compound is administered to the subject every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compound disclosed herein, or a pharmaceutically acceptable salt thereof is administered to animals. In some embodiments, the compounds are administered orally or parenterally, including the intravenous, intramuscular, intraperitoneal, subcutaneous, rectal, and topical routes of administration.

In another aspect, provided herein are pharmaceutical compositions comprising a compound described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compounds described herein, or a pharmaceutically acceptable salt thereof are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt thereof. In some embodiments, the pharmaceutical composition comprises a liquid carrier. In some embodiments, the liquid carrier is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

### Combination

Disclosed herein are methods of treating a disease or disorder associated with modulating autophagy via inhibition of ULK1 and/or ULK2, with a compound disclosed herein, or a pharmaceutically acceptable salt thereof, in combination with an additional therapeutic agent.

In some embodiments, the additional therapeutic agent is administered at the same time as the compound disclosed herein. In some embodiments, the additional therapeutic agent and the compound disclosed herein are administered sequentially. In some embodiments, the additional therapeutic agent is administered less frequently than the compound disclosed herein. In some embodiments, the additional therapeutic agent is administered more frequently than the compound disclosed herein. In some embodiments, the additional therapeutic agent is administered prior than the administration of the compound disclosed herein. In some embodiments, the additional therapeutic agent is administered after the administration of the compound disclosed herein.

In some embodiments, the additional therapeutic agent is an additional anticancer agent. Such additional anticancer agents include compounds derived from the following classes: mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases and/or phosphatases, cell cycle inhibitors, biological response modifiers, enzyme inhibitors, antisense oligonucleotides or oligonucleotide derivatives, cytotoxics, immuno-oncology agents, and the like. In some embodiments, the additional anti-cancer agent is a tyrosine kinase inhibitor. In some embodiments, the tyrosine kinase inhibitor is selected from imatinib and nilotinib. In some embodiments the additional therapeutic agent is radiotherapy.

In some embodiments, the additional therapeutic agent is a poly ADP ribose polymerase (PARP) inhibitor. In some embodiments, the PARP inhibitor is olaparib, rucaparib, niraparib, or talazoparib.

In some embodiments, the additional therapeutic agent is a BRAF inhibitor. In some embodiments, the BRAF inhibitor is encorafenib, dabrafenib, or vemurafenib.

In some embodiments, the additional therapeutic agent is an ERK inhibitor. In some embodiments, the ERK inhibitor is ulixertinib, ASN007, LY3214996, AZ13767370, MK-8353, or LTT462.

In some embodiments, the additional therapeutic agent is a MEK inhibitor. In some embodiments, the MEK inhibitor is trametinib, binimetinib, cobimetinib, or selumetinib.

In some embodiments, the additional therapeutic agent is mammalian target of rapamycin inhibitor (mTOR). In some embodiments, the mTOR inhibitor is sirolimus, everolimus, temsirolimus, or ridaforolimus (AP23573 and MK-8669).

In some embodiments, the additional therapeutic agent is an anti-angiogenesis agent. In some embodiments, the additional therapeutic agent is a VEGF inhibitor, VEGFR inhibitor, PDGFR inhibitor, sunitinib, bevacizumab, axitinib, SU 14813 (Pfizer), or AG 13958 (Pfizer). In some embodiments, the additional therapeutic agent is sorafenib.

In some embodiments, the additional therapeutic agent is a so-called signal transduction inhibitor (e.g., inhibiting the means by which regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, farnesyl protein transferase inhibitors, EGF inhibitor, ErbB-1 (EGFR), ErbB-2, pan erb, ERBB family inhibitors, IGF1R inhibitors, MEK, c-Kit inhibitors, Erk1/2 inhibitors, FLT-3 inhibitors, K-Ras inhibitors, PI3 kinase inhibitors, JAK inhibitors, STAT inhibitors, Raf kinase inhibitors, Akt inhibitors, mTOR inhibitor, P70S6 kinase inhibitors, inhibitors of the WVNT pathway and so called multi-targeted kinase inhibitors.

In some embodiments, the additional therapeutic agent is a tyrosine kinase inhibitor. In some embodiments, the tyrosine kinase inhibitor is selected from imatinib and nilotinib.

### EXAMPLES

### Example 1: 5-(2-((7-chloro-1,2,3,4-tetrahydroisoquinolin-6-yl)amino)-5-(trifluoromethyl)pyrimidin-4-yl)thiophene-3-carboxamide

### Step 1: N-(4-chlorophenethyl)-2,2,2-trifluoroacetamide

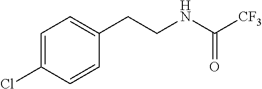 [SMILES: O=C(NCCc1ccc(Cl)cc1)C(F)(F)F]

2-(4-Chlorophenyl)ethylamine (642 mmol) was dissolved in dichloromethane (1.2 L) and triethylamine (107 mL) was added in one portion. The mixture was cooled down to 0° C. and trifluoroacetic anhydride (771 mmol) was added dropwise over 40 minutes while maintaining the reaction temperature below 5° C. The reaction mixture was stirred at 0-5° C. for 15 minutes and quenched with water. The layers were separated and the aqueous layer was extracted with dichloromethane twice. The combined organic layers were combined, washed with brine, dried over anhydrous sodium sulfate, filtered, and evaporated to afford the title compound in 99% yield. This material was used in the next step without purification. m/z (ESI, −ve)=250.6 (M−H).

### Step 2: 1-(7-chloro-3,4-dihydroisoquinolin-2(1H)-yl)-2,2,2-trifluoroethan-1-one

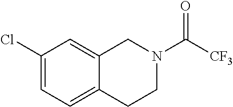 [SMILES: O=C(N1CCc2ccc(Cl)cc2C1)C(F)(F)F]

N-[2-(4-chlorophenyl)ethyl]-2,2,2-trifluoroacetamide (636.56 mmol) was suspended in acetic acid (173 mL) and paraformaldehyde (1.27 mol) was added in one portion. The mixture was cooled down to 10° C. and concentrated sulfuric acid (218 mL) was added dropwise over 85 minutes maintaining the temperature below 15° C. The reaction mixture was stirred at 50-60° C. for 1 hour and then cooled down to room temperature and stirred for another 16 hours. The reaction mixture was poured on ice and the aqueous layer was separated and extracted with dichloromethane three times. The combined organic layers were combined, washed with a saturated solution of sodium carbonate, dried over sodium sulfate, and evaporated to afford a crude that was triturated with 10% ethyl acetate in hexane, filtered, washed with n-hexane and dried. The title compound was isolated in 60% yield. m/z (ESI, +ve)=264.1 [M+H]⁺.

### Step 3: 1-(7-chloro-6-nitro-3,4-dihydroisoquinolin-2(1H)-yl)-2,2,2-trifluoroethan-1-one

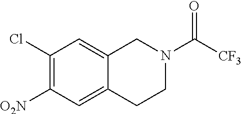 [SMILES: O=C(N1CCc2cc([N+](=O)[O-])c(Cl)cc2C1)C(F)(F)F]

1-(7-chloro-1,2,3,4-tetrahydroisoquinolin-2-yl)-2,2,2-trifluoroethan-1-one (152 mmol) was dissolved in sulfuric acid (374 mL). The solution was cooled down to −20° C. and fuming nitric acid (6.5 mL) was added dropwise over 30 min while keeping the temperature at −20° C. After 15 minutes, the reaction was quenched with ice-water (1.5 L) and the aqueous layer was extracted with dichloromethane three times. The combined organic layers were dried over anhydrous sodium sulfate, filtered, and concentrated to afford the title compound in 79% yield. m/z (ESI, −ve)=307.1 (M−H)−.

### Step 4: 1-(6-amino-7-chloro-3,4-dihydroisoquinolin-2(1H)-yl)-2,2,2-trifluoroethan-1-one

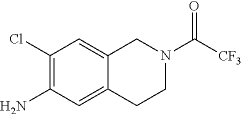 [SMILES: Nc1cc2c(cc1Cl)CN(C(=O)C(F)(F)F)CC2]

To a solution of 1-(7-chloro-6-nitro-1,2,3,4-tetrahydroisoquinolin-2-yl)-2,2,2-trifluoroethan-1-one (120 mmol) in ethanol (928 mL), iron powder (608 mmol) was added in small portions. Acetic acid (14 mL) and 6N aqueous HCl (5 mL) were added and the resulting reaction mixture was heated at 70° C. for 2 hours. The crude reaction mixture was cooled down to room temperature, filtered through a pad of celite and rinsed with ethanol. The filtrate was concentrated to obtain a dark brown semisolid that was purified by chromatography (20% ethyl acetate in hexanes) to afford the title compound in 96% yield. m/z (ESI, +ve)=279.1 [M+H]⁺.

### Step 5: 1-(7-chloro-6-((4-chloro-5-(trifluoromethyl)pyrimidin-2-yl)amino)-3,4-dihydroisoquinolin-2(1H)-yl)-2,2,2-trifluoroethan-1-one

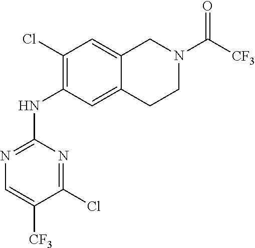 [SMILES: O=C(N1CCc2cc(Nc3ncc(C(F)(F)F)c(Cl)n3)c(Cl)cc2C1)C(F)(F)F]

1-(6-amino-7-chloro-1,2,3,4-tetrahydroisoquinolin-2-yl)-2,2,2-trifluoroethan-1-one (34.7 mmol) was dissolved in 2,4-dichloro-5-(trifluoromethyl)pyrimidine (191 mmol) and the mixture was stirred at 70° C. for 24 hours. The reaction mixture was diluted with dichloromethane, celite was added and the solvent was evaporated under reduced pressure. Purification by silica gel chromatography (20% ethyl acetate in hexanes) afforded the title compound in 37% yield. m/z (ESI, +ve)=459.1 [M+H]⁺.

### Step 6: Methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate

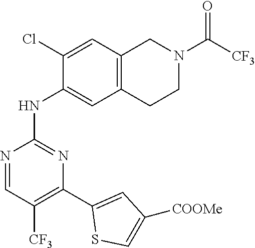 [SMILES: COC(=O)c1csc(-c2nc(Nc3cc4c(cc3Cl)CN(C(=O)C(F)(F)F)CC4)ncc2C(F)(F)F)c1]

A solution of 1-(7-chloro-6-((4-chloro-5-(trifluoromethyl)pyrimidin-2-yl)amino)-3,4-dihydroisoquinolin-2(1H)-yl)-2,2,2-trifluoroethan-1-one (0.41 mmol), triethylamine (0.83 mmol), Pd(dppf)Cl₂ (0.041 mmol) and 4-(methoxycarbonyl)thiophene-2-boronic acid pinacol ester (0.50 mmol) in 1,4-dioxane (2.0 mL) and water (0.4 mL) was stirred at 90° C. for 16 hours. The reaction mixture was concentrated under reduced pressure and the resulting residue was purified by silica gel chromatography (70% ethyl acetate in hexanes) to afford the title compound in 40% yield. m/z (ESI, −ve)=563.3 (M−H)−.

### Step 7

Methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate (0.88 mmol) was suspended in 7N solution of ammonia in methanol (2.53 mL). The reaction mixture was stirred at 90° C. for 48 hours and the volatiles removed under reduced pressure to afford a residue that was triturated with methanol and purified by HPLC. The title compound was isolated in 30% yield. m/z (ESI, +ve)=454.1 [M+H]⁺. ¹H NMR (400 MHz, DMSO-d₆) δ 9.86 (s, 1H), 8.78 (s, 1H), 8.42 (d, J=1.2 Hz, 1H), 8.22 (s, 1H), 8.03 (s, 1H), 7.96 (s, 1H), 7.39 (s, 1H), 7.37 (s, 1H), 7.29 (s, 1H), 3.96 (s, 2H), 3.06 (t, J=6.0 Hz, 2H), 2.77 (t, J=5.8 Hz, 2H).

### Example 2: 5-(2-((7-chloro-1,2,3,4-tetrahydroisoquinolin-6-yl)amino)-5-(trifluoromethyl)pyrimidin-4-yl)-N,N-dimethylthiophene-3-carboxamide

### Step 1: 5-(2-((7-chloro-1,2,3,4-tetrahydroisoquinolin-6-yl)amino)-5-(trifluoromethyl)pyrimidin-4-yl)thiophene-3-carboxylic acid

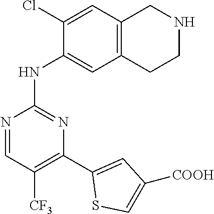 [SMILES: O=C(O)c1csc(-c2nc(Nc3cc4c(cc3Cl)CNCC4)ncc2C(F)(F)F)c1]

A solution of methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate (0.16 mmol) in ethanol (1.9 mL), was treated with a 4M aqueous solution of LiOH (0.19 mL) and the reaction mixture was stirred at room temperature for 1 hour. Evaporation of volatiles under reduced pressure afforded the title compound in quantitative yield. This material was used in the next step without further purification. m/z (ESI, +ve)=455.1 [M+H]⁺

### Step 2: 5-(2-((2-(tert-butoxycarbonyl)-7-chloro-1,2,3,4-tetrahydroisoquinolin-6-yl)amino)-5-(trifluoromethyl)pyrimidin-4-yl)thiophene-3-carboxylic acid

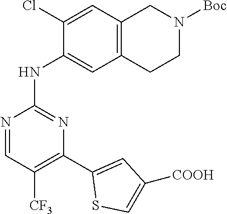 [SMILES: CC(C)(C)OC(=O)N1CCc2cc(Nc3ncc(C(F)(F)F)c(-c4cc(C(=O)O)cs4)n3)c(Cl)cc2C1]

To a solution of 5-(2-((7-chloro-1,2,3,4-tetrahydroisoquinolin-6-yl)amino)-5-(trifluoromethyl)pyrimidin-4-yl)thiophene-3-carboxylic acid (0.16 mmol) in acetonitrile (3.7 mL) was added di-tert-butyl dicarbonate (0.33 mmol) and triethylamine (0.069 mL). The reaction mixture was stirred at room temperature for 1 hour and the volatiles were removed under reduced pressure to afford the title compound in quantitative yield. This material was used in the next step without further purification. m/z (ESI, −ve)=553.3 (M−H)−

### Step 3: tert-butyl 7-chloro-6-((4-(4-(dimethylcarbamoyl)thiophen-2-yl)-5-(trifluoromethyl)pyrimidin-2-yl)amino)-3,4-dihydroisoquinoline-2(1H)-carboxylate

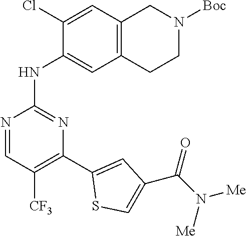 [SMILES: CN(C)C(=O)c1csc(-c2nc(Nc3cc4c(cc3Cl)CN(C(=O)OC(C)(C)C)CC4)ncc2C(F)(F)F)c1]

A solution of O-(1H-6-chlorobenzotriazole-1-yl)-1,1,3,3-tetramethyluronium hexafluorophosphate (HCTU) (0.33 mmol), 5-(2-((2-(tert-butoxycarbonyl)-7-chloro-1,2,3,4-tetrahydroisoquinolin-6-yl)amino)-5-(trifluoromethyl)pyrimidin-4-yl)thiophene-3-carboxylic acid (0.16 mmol), triethylamine (0.046 mL) and methylamine (2M in THF, 0.165 mL) in acetonitrile (1.8 mL) was stirred at room temperature for 30 minutes. Evaporation of volatiles afforded a residue that was dissolved in dichloromethane and water. The organic layer was separated and dried over anhydrous sodium sulfate and concentrated. The crude product was purified by silica gel chromatography (70% hexanes in ethyl acetate) to afford the title compound in 93% yield. m/z (ESI, −ve)=580.5 (M−H)−.

### Step 4

A solution of tert-butyl 7-chloro-6-((4-(4-(dimethylcarbamoyl)thiophen-2-yl)-5-(trifluoromethyl)pyrimidin-2-yl)amino)-3,4-dihydroisoquinoline-2(1H)-carboxylate (0.12 mmol) in 1,4-dioxane (1.4 mL) was treated with a 4N aqueous solution of HCl (1.4 mL) and the solution was stirred at room temperature overnight. Evaporation of volatiles afforded a residue that was purified by HPLC. The title compound was isolated in 19% yield. m/z (ESI, +ve)=482.2 [M+H]⁺. ¹H NMR (400 MHz, DMSO-d₆) δ 9.99 (s, 1H), 8.97 (s, 2H), 8.80 (s, 1H), 8.15 (d, J=1.3 Hz, 1H), 7.77 (d, J=1.3 Hz, 1H), 7.53 (s, 1H), 7.50 (s, 1H), 4.31 (s, 2H), 3.42 (t, J=6.3 Hz, 2H), 3.02 (m, 8H).

### Example 3: 5-(2-((7-chloro-1,2,3,4-tetrahydroisoquinolin-6-yl)amino)-5-(trifluoromethyl)pyrimidin-4-yl)-N,N-dimethylthiophene-2-carboxamide

### Step 1: 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-2-carboxylic acid

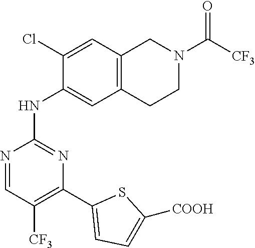 [SMILES: O=C(O)c1ccc(-c2nc(Nc3cc4c(cc3Cl)CN(C(=O)C(F)(F)F)CC4)ncc2C(F)(F)F)s1]

The title compound was prepared analogously to Example 1, step 6, where 4-(methoxycarbonyl)thiophene-2-boronic acid pinacol ester was substituted with 5-carboxylthiophene-2-boronic acid pinacol ester. The title compound was isolated in 23% yield. m/z (ESI, +ve)=551.3 [M+H]⁺.

### Step 2

5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-2-carboxylic acid (0.15 mmol) was treated with SOCl₂ (0.11 mL) and the mixture was stirred at 60° C. for 1 hour and concentrated to dryness. The resulting residue was dissolved in THF (2.4 mL) and a 2M solution of dimethylamine in THF (0.76 mL) was added. This mixture was stirred at room temperature for 1 hour. Potassium carbonate (0.76 mmol) and water (0.6 mL) were added and the reaction mixture was stirred at 50° C. overnight, concentrated under reduced pressure and the residue purified by preparative HPLC to afford the title compound in 2% yield. m/z (ESI, +ve)=482.14 [M+H]⁺. ¹H NMR (300 MHz, DMSO-d₆) δ 8.78 (s, 1H), 8.48 (s, 1H), 7.64 (d, J=3.9 Hz, 1H), 7.54 (d, J=4.1 Hz, 1H), 7.30 (s, 1H), 7.22 (s, 1H), 3.84 (s, 2H), 2.94 (t, J=5.8 Hz, 2H), 2.68 (d, J=5.6 Hz, 2H).

### Example 4: 2-(2-((7-chloro-1,2,3,4-tetrahydroisoquinolin-6-yl)amino)-5-(trifluoromethyl)pyrimidin-4-yl)-6,7-dihydrothieno[3,2-c]pyridin-4(5H)-one

### Step 1: 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one

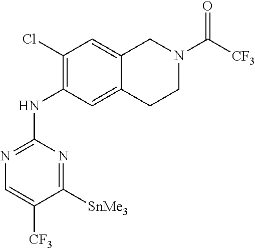

A solution of 1-(7-chloro-6-((4-chloro-5-(trifluoromethyl)pyrimidin-2-yl)amino)-3,4-dihydroisoquinolin-2(1H)-yl)-2,2,2-trifluoroethan-1-one (3.26 mmol), 1,4-bis(diphenylphosphino)butane (0.65 mmol), palladium (II) acetate (0.65 mmol) and hexamethylditin (9.80 mmol) in 1,4-dioxane (30 mL) was stirred at 95° C. for 24 hours. The reaction mixture was filtered through a pad of celite and the filtrate was diluted with ethyl acetate and water. The layers were separated and the aqueous layer was extracted with ethyl acetate three times. The combined organic layers were washed with brine, dried over anhydrous sodium sulfate and concentrated. The crude material was purified by silica gel chromatography (hexanes with 10% of triethylamine) to afford the title compound in 32% yield. m/z (ESI, +ve)=590.0 [M+H]⁺. ¹H NMR (400 MHz, DMSO-d₆) δ 9.47 (s, 1H), 8.56 (s, 1H), 7.60-7.47 (m, 2H), 4.77 (d, J=12.4 Hz, 3H), 3.82 (q, J=5.8 Hz, 2H), 2.95-2.85 (m, 2H), 0.31 (s, 9H).

### Step 2: 2-(2-((7-chloro-2-(2,2,2-trifluoroacetyl)-1,2,3,4-tetrahydroisoquinolin-6-yl)amino)-5-(trifluoromethyl)pyrimidin-4-yl)-6,7-dihydrothieno[3,2-c]pyridin-4(5H)-one

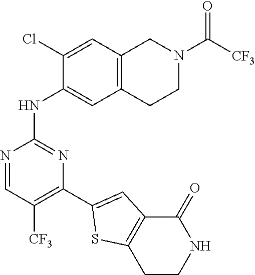 [SMILES: O=C1NCCc2sc(-c3nc(Nc4cc5c(cc4Cl)CN(C(=O)C(F)(F)F)CC5)ncc3C(F)(F)F)cc21]

A mixture of CuI (0.037 mmol), PdCl₂(PPh₃)₂ (0.009 mmol), 2-bromo-6,7-dihydrothieno[3,2-C]pyridin-4(5H)-one (0.22 mmol) and 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one (0.18 mmol) in 1,4-dioxane (2.8 mL) was stirred at 100° C. for 2 hours. The reaction mixture was cooled down to room temperature and concentrated under reduced pressure. The residue was purified by silica gel chromatography (30% ethyl acetate in hexanes) to afford the title compound in 47% yield. m/z (ESI, +ve)=576.2 [M+H]⁺.

### Step 3

Potassium carbonate (0.36 mmol) was added to a stirred solution of 2-(2-((7-chloro-2-(2,2,2-trifluoroacetyl)-1,2,3,4-tetrahydroisoquinolin-6-yl)amino)-5-(trifluoromethyl)pyrimidin-4-yl)-6,7-dihydrothieno[3,2-c]pyridin-4(5H)-one (0.073 mmol) in a mixture of ethanol (1.2 mL) and water (0.12 mL) and the reaction mixture was stirred at 70° C. for 1 hour. Evaporation of volatiles afforded a residue that was purified by HPLC. The title compound was isolated in 21% yield. m/z (ESI, +ve)=480.0[M+H]⁺. ¹H NMR (400 MHz, DMSO-d₆) δ 8.75 (s, 1H), 8.25 (s, 2H), 7.88-7.75 (m, 2H), 7.34 (s, 1H), 7.26 (s, 1H), 3.92 (m, 2H), 3.04 (m, 6H), 2.74 (m, 2H).

### Example 5: 5-(2-((2-(azetidin-3-yl)-7-chloro-1,2,3,4-tetrahydroisoquinolin-6-yl)amino)-5-(trifluoromethyl)pyrimidin-4-yl)thiophene-3-carboxamide

### Step 1: tert-butyl 3-(6-((4-(4-carbamoylthiophen-2-yl)-5-(trifluoromethyl)pyrimidin-2-yl)amino)-7-chloro-3,4-dihydroisoquinolin-2(1H)-yl)azetidine-1-carboxylate

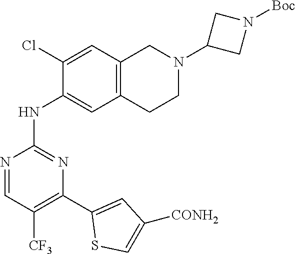 [SMILES: CC(C)(C)OC(=O)N1CC(N2CCc3cc(Nc4ncc(C(F)(F)F)c(-c5cc(C(N)=O)cs5)n4)c(Cl)cc3C2)C1]

To a stirred solution of tert-butyl 3-oxoazetidine-1-carboxylate (0.41 mmol) and 5-(2-((7-chloro-1,2,3,4-tetrahydroisoquinolin-6-yl)amino)-5-(trifluoromethyl)pyrimidin-4-yl)thiophene-3-carboxamide (0.34 mmol) in dichloromethane (3.4 mL), triethylamine (0.49 mL) and NaBH(OAc)₃ (1.69 mmol) were added in one portion. The reaction mixture was stirred at room temperature overnight, diluted with water and the organic layer separated, dried over sodium sulfate, and concentrated to dryness. The crude material was purified by silica gel chromatography (0 to 100% ethyl acetate in hexanes) to afford the title compound in 93% yield. m/z (ESI, +ve)=609.3 [M+H]⁺.

### Step 2

A solution of tert-butyl 3-(6-((4-(4-carbamoylthiophen-2-yl)-5-(trifluoromethyl)pyrimidin-2-yl)amino)-7-chloro-3,4-dihydroisoquinolin-2(1H)-yl)azetidine-1-carboxylate (0.071 mmol) was treated with a 4N solution of HCl in 1,4-dioxane (0.8 mL). The reaction was stirred at room temperature for 1 hour, the volatiles were removed under reduced pressure and the resulting crude material was purified by preparative HPLC to afford the title compound in 40% yield. m/z (ESI, +ve)=509.4 [M+H]⁺.

### Example 6: 5-(2-((7-chloro-1,2,3,4-tetrahydroisoquinolin-6-yl)amino)-5-(trifluoromethyl)pyrimidin-4-yl)-N-methylthiophene-3-carboxamide

### Step 1: tert-butyl 7-chloro-6-((4-(4-(methylcarbamoyl)thiophen-2-yl)-5-(trifluoromethyl)pyrimidin-2-yl)amino)-3,4-dihydroisoquinoline-2(1H)-carboxylate

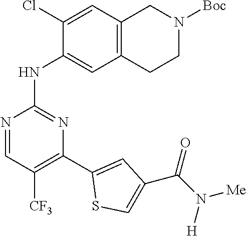 [SMILES: CNC(=O)c1csc(-c2nc(Nc3cc4c(cc3Cl)CN(C(=O)OC(C)(C)C)CC4)ncc2C(F)(F)F)c1]

The title compound was prepared analogously to Example 3, where the solution of dimethylamine was replaced with a 2M solution of methylamine in THF. The title compound was isolated in 53% yield. m/z (ESI, −ve)=566.6 (M−H)−.

### Step 2

The title compound was prepared analogously to Example 2 where tert-butyl 7-chloro-6-((4-(4-(dimethylcarbamoyl)thiophen-2-yl)-5-(trifluoromethyl)pyrimidin-2-yl)amino)-3,4-dihydroisoquinoline-2(1H)-carboxylate was replaced with tert-butyl 7-chloro-6-((4-(4-(methylcarbamoyl)thiophen-2-yl)-5-(trifluoromethyl)pyrimidin-2-yl)amino)-3,4-dihydroisoquinoline-2(1H)-carboxylate. The title compound was isolated in 40% yield. m/z (ESI, +ve)=468.10 [M+H]⁺. ¹H NMR (400 MHz, DMSO-d₆) δ 9.84 (s, 1H), 8.76 (s, 1H), 8.47 (d, J=4.6 Hz, 1H), 8.42-8.28 (m, 3H), 8.01 (s, 1H), 7.31 (s, 1H), 7.23 (s, 1H), 3.87 (s, 3H), 2.97 (s, 3H), 2.75 (d, J=4.5 Hz, 3H), 2.70 (s, 2H).

### Example 7: 5-(2-((6-chloroisoindolin-5-yl)amino)-5-(trifluoromethyl)pyrimidin-4-yl)thiophene-3-carboxamide

### Step 1: 1-(5-chloroisoindolin-2-yl)-2,2,2-trifluoroethan-1-one

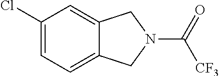 [SMILES: O=C(N1Cc2ccc(Cl)cc2C1)C(F)(F)F]

5-Chloroisoindoline (5.69 mmol) and triethylamine (2.4 mL) were dissolved in dichloromethane (28 mL). The solution was cooled down to −10° C. and trifluoroacetic anhydride (8.53 mmol) was added dropwise. The reaction mixture was allowed to warm up to room temperature, stirred at that temperature for 1 hour and quenched with water. The organic layer was separated and dried over anhydrous sodium sulfate, filtered, and concentrated to afford the title compound in 77% yield, which was used in the next step without further purification. m/z (ESI, +ve)=250.0 [M+H]⁺

### Step 2: 1-(5-chloro-6-nitroisoindolin-2-yl)-2,2,2-trifluoroethan-1-one

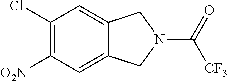 [SMILES: O=C(N1Cc2cc(Cl)c([N+](=O)[O-])cc2C1)C(F)(F)F]

1-(5-Chloro-2,3-dihydro-1H-isoindol-2-yl)-2,2,2-trifluoroethan-1-one (4.37 mmol) was stirred in H₂SO₄ (12 mL) at 0° C. until the starting material is completely dissolved. The solution was cooled down to −20° C. and 90% fuming HNO₃ (0.43 mL) was added. The reaction mixture was stirred at −20° C. for 1 hour and quenched by addition of ice. The mixture was extracted with dichloromethane three times and the combined organic layers were washed with a saturated solution of NaHCO₃, dried over anhydrous sodium sulfate, filtered, and concentrated to afford the title compound in 98% yield, which was used in the next step without purification. m/z (ESI, +ve)=293.1 [M+H]⁺.

### Step 3: 1-(5-amino-6-chloroisoindolin-2-yl)-2,2,2-trifluoroethan-1-one

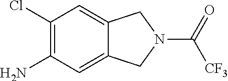 [SMILES: Nc1cc2c(cc1Cl)CN(C(=O)C(F)(F)F)C2]

1-(5-chloro-6-nitro-2,3-dihydro-1H-isoindol-2-yl)-2,2,2-trifluoroethan-1-one (4.23 mmol), acetic acid (0.50 mL) and iron powder (21.3 mmol) were suspended in EtOH (63.5 mL). The mixture was stirred at 80° C. for 1.5 hours and the volatiles were removed under reduced pressure to afford a residue that was filtered through a pad of celite and rinsed with ethyl acetate. Evaporation of volatiles afforded the title compound in 96% yield. m/z (ESI, +ve)=265.0 [M+H]⁺.

### Step 4: 1-(5-chloro-6-((4-chloro-5-(trifluoromethyl)pyrimidin-2-yl)amino)isoindolin-2-yl)-2,2,2-trifluoroethan-1-one

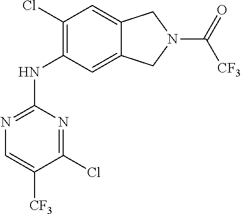 [SMILES: O=C(N1Cc2cc(Cl)c(Nc3ncc(C(F)(F)F)c(Cl)n3)cc2C1)C(F)(F)F]

A mixture of 1-(5-amino-6-chloro-2,3-dihydro-1H-isoindol-2-yl)-2,2,2-trifluoroethan-1-one (4.12 mmol) and 2,4-dichloro-5-(trifluoromethyl)pyrimidine (28.8 mmol) was stirred at 60° C. overnight. The reaction mixture was diluted with dichloromethane (3 mL) and the insoluble material was filtered off. The filtrate was concentrated and purified by chromatography in silica gel (50-100% dichloromethane in hexanes) to afford the title compound in 26% yield. m/z (ESI, +ve)=445.7 [M+H]⁺.

### Step 5: Methyl-5-(2-((6-chloro-2-(2,2,2-trifluoroacetyl)isoindolin-5-yl)amino)-5-(trifluoromethyl)pyrimidin-4-yl)thiophene-3-carboxylate

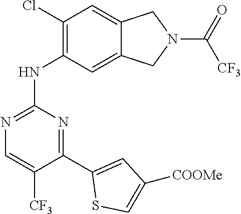 [SMILES: COC(=O)c1csc(-c2nc(Nc3cc4c(cc3Cl)CN(C(=O)C(F)(F)F)C4)ncc2C(F)(F)F)c1]

The title compound was prepared analogously to Example 1, step 6 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with 1-(5-chloro-6-((4-chloro-5-(trifluoromethyl)pyrimidin-2-yl)amino)isoindolin-2-yl)-2,2,2-trifluoroethan-1-one. The title compound was isolated in 72% yield. m/z (ESI, +ve)=551.9 [M+H]⁺.

### Step 6

The title compound was prepared analogously to Example 1 where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with methyl-5-(2-((6-chloro-2-(2,2,2-trifluoroacetyl)isoindolin-5-yl)amino)-5-(trifluoromethyl)pyrimidin-4-yl)thiophene-3-carboxylate. The title compound was isolated in 83% yield. m/z (ESI, +ve)=440.1 [M+H]⁺. ¹H NMR (400 MHz, DMSO-d₆) δ 9.89 (s, 1H), 8.76 (s, 1H), 8.41 (d, J=1.2 Hz, 1H), 8.23 (s, 1H), 8.02 (s, 1H), 7.96 (s, 1H), 7.49 (s, 1H), 7.47 (s, 1H), 7.38 (s, 1H), 4.14 (s, 4H).

### Example 8: 7-chloro-N-(4-(4-(4,5-dihydrooxazol-2-yl)thiophen-2-yl)-5-(trifluoromethyl)pyrimidin-2-yl)-1,2,3,4-tetrahydroisoquinolin-6-amine

### Step 1: 5-bromothiophene-3-carboxylic acid

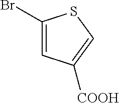

Methyl 2-bromothiophene-4-carboxylate (13.6 mmol) and LiOH (20.4 mmol) were dissolved in THF (30 mL) and water (30 mL) and the solution stirred at 50° C. for 2 hours. The THF was removed under reduced pressure and the aqueous solution was acidified with concentrated aqueous HCl to pH=3. The resulting white solid was filtered, washed with water, and dried under high vacuum. The title compound was isolated in 87% yield. m/z (ESI, −ve)=206.9 (M−H)−.

### Step 2: 5-Bromo-N-(2-hydroxyethyl)thiophene-3-carboxamide

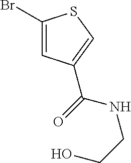 [SMILES: O=C(NCCO)c1csc(Br)c1]

Over a solution of 5-bromothiophene-3-carboxylic acid (11.4 mmol) in dichloromethane (47 mL) and 3 drops of DMF at 0° C., oxalyl chloride (17.1 mmol) was added. The resulting mixture was stirred at room temperature for 30 minutes, the volatiles were removed under reduced pressure and the residue re-dissolved in dichloromethane (25 mL). This new solution was added over a solution of ethanolamine (0.76 mL) and triethylamine (3.2 mL) in dichloromethane (25 mL) precooled to 0° C. The reaction was allowed to warm up to room temperature and stirred for one hour. Evaporation of volatiles and purification of the crude material by silica gel (0-100% ethyl acetate in hexanes) afforded the title compound in 46% yield. m/z (ESI, +ve)=251.9 [M+H]⁺.

### Step 3: 2-(5-Bromothiophen-3-yl)-4,5-dihydrooxazole

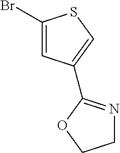 [SMILES: Brc1cc(C2=NCCO2)cs1]

5-Bromo-N-(2-hydroxyethyl)thiophene-3-carboxamide (1.9 mmol) was suspended in dichloromethane (10 mL). Triethylamine (0.69 mL) was added and the mixture was cooled to 0° C. Methanesulfonyl chloride (0.19 mL) was added dropwise and the reaction mixture was allowed to warm to room temperature and stirred for 12 hours. Evaporation of volatiles under reduced pressure afforded a residue that was purified by silica gel chromatography (0-10% methanol in dichloromethane) to afford the title compound in 50% yield. m/z (ESI, +ve)=234.4 [M+H]⁺.

### Step 4: 1-(7-Chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one

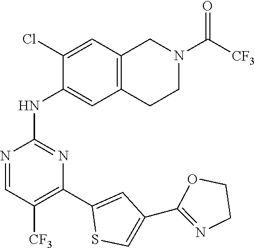 [SMILES: O=C(N1CCc2cc(Nc3ncc(C(F)(F)F)c(-c4cc(C5=NCCO5)cs4)n3)c(Cl)cc2C1)C(F)(F)F]

The title compound was prepared analogously to Example 4, step 2 where 2-bromo-6,7-dihydrothieno[3,2-C]pyridin-4(5H)-one was replaced with 2-(5-bromothiophen-3-yl)-4,5-dihydrooxazole. The title compound was isolated in 32% yield. m/z (ESI, +ve)=576.8 [M+H]⁺.

### Step 5

1-(7-Chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one (0.064 mmol) was suspended in a 7N solution of ammonia in methanol (2 mL) and the reaction mixture was stirred at 50° C. for 2 hours. The solvent was removed under reduced pressure and the crude was purified by preparative TLC (dichloromethane with 5% of a 3M solution of ammonia in methanol) to afford the title compound in 32% yield. m/z (ESI, +ve)=479.87 [M+H]⁺. ¹H NMR (400 MHz, DMSO-d₆) δ 9.86 (s, 1H), 8.78 (s, 1H), 8.34 (d, J=1.1 Hz, 1H), 8.01 (s, 1H), 7.27 (d, J=32.8 Hz, 2H), 4.39 (t, J=9.5 Hz, 2H), 3.94 (t, J=9.4 Hz, 2H), 3.85 (s, 1H), 2.95 (t, J=5.8 Hz, 2H), 2.73-2.63 (m, 2H).

### Example 9: 5-(2-((7-ethyl-1,2,3,4-tetrahydroisoquinolin-6-yl)amino)-5-(trifluoromethyl)pyrimidin-4-yl)thiophene-3-carboxamide

### Step 1: 7-Ethyl-6-nitro-1,2,3,4-tetrahydroisoquinoline

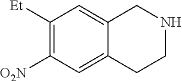 [SMILES: CCc1cc2c(cc1[N+](=O)[O-])CCNC2]

1-(7-chloro-6-nitro-1,2,3,4-tetrahydroisoquinolin-2-yl)-2,2,2-trifluoroethan-1-one (3.24 mmol), Pd(dppf)₂Cl₂ complex with dichloromethane (0.16 mmol), potassium phosphate tribasic (16.2 mmol), 1,1′-Bis(diphenylphosphino)ferrocene (0.26 mmol) and ethylboronic acid (9.72 mmol) were suspended in toluene (20 mL) and water (3 mL) and the mixture was stirred at 100° C. overnight. The reaction was cooled down to room temperature and washed with water and 1N aqueous HCl. The pH of the organic layer was made alkaline (pH>10) by the addition 15% aqueous solution of NaOH, the organic layer was separated and the aqueous layer was extracted with dichloromethane twice. The combined organic layers were dried over anhydrous sodium sulfate, filtered and concentrated to afford the title compound in 61% yield. m/z (ESI, +ve)=207.1 [M+H]⁺.

### Step 2: 1-(7-ethyl-6-nitro-3,4-dihydroisoquinolin-2(1H)-yl)-2,2,2-trifluoroethan-1-one

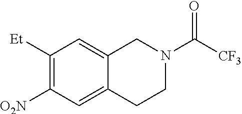 [SMILES: CCc1cc2c(cc1[N+](=O)[O-])CCN(C(=O)C(F)(F)F)C2]

A −10° C. solution of 7-ethyl-6-nitro-1,2,3,4-tetrahydroisoquinoline (1.96 mmol) and triethylamine (0.55 mL) in dichloromethane (9 mL) was treated with trifluoroacetic anhydride (2.94 mmol) and the resulting mixture was stirred at room temperature for one hour. The reaction was quenched with water and the organic layer was separated, dried over anhydrous sodium sulfate, filtered and concentrated. The crude material was purified by silica gel chromatography (10-60% ethyl acetate in hexanes) to afford the title compound in 67% yield. m/z (ESI, −ve)=301.2 (M−H)−.

### Step 3: 1-(6-Amino-7-ethyl-3,4-dihydroisoquinolin-2(1H)-yl)-2,2,2-trifluoroethan-1-one

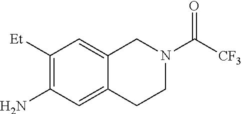 [SMILES: CCc1cc2c(cc1N)CCN(C(=O)C(F)(F)F)C2]

A mixture of 1-(7-ethyl-6-nitro-1,2,3,4-tetrahydroisoquinolin-2-yl)-2,2,2-trifluoroethan-1-one (1.31 mmol), iron powder (6.55 mmol) and acetic acid (0.15 mL) was suspended in ethanol (20 mL) and heated at 80° C. overnight. Evaporation of volatiles under reduced pressure afforded a residue that was suspended in ethyl acetate and filtered through silica gel. The filtrate was concentrated and the crude material was purified by silica gel chromatography (10-60% ethyl acetate in hexanes) to afford the title compound in 58% yield. m/z (ESI, +ve)=273.2 [M+H]⁺

### Step 4: 1-(6-((4-Chloro-5-(trifluoromethyl)pyrimidin-2-yl)amino)-7-ethyl-3,4-dihydroisoquinolin-2(1H)-yl)-2,2,2-trifluoroethan-1-one

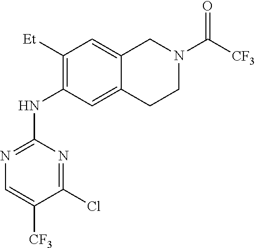 [SMILES: CCc1cc2c(cc1Nc1ncc(C(F)(F)F)c(Cl)n1)CCN(C(=O)C(F)(F)F)C2]

A solution of 1-(6-Amino-7-ethyl-3,4-dihydroisoquinolin-2(1H)-yl)-2,2,2-trifluoroethan-1-one (0.73 mmol) in 2,4-dichloro-5-(trifluoromethyl)pyrimidine (4.70 mmol) was heated at 50° C. overnight. The insoluble materials were filtered and the solvent was removed under reduced pressure. Purification by silica gel chromatography afforded the title compound in 39% yield. m/z (ESI, +ve)=453.9 [M+H]⁺.

### Step 5

The title compound was prepared analogously to Example 1 where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate. The title compound was isolated in 26% yield. m/z (ESI, +ve)=448.2 [M+H]⁺. ¹H NMR (400 MHz, DMSO-d₆) δ 9.67 (s, 1H), 8.71 (s, 1H), 8.40 (s, 1H), 8.28 (s, 1H), 8.00 (s, 1H), 7.95 (s, 1H), 7.38 (s, 1H), 7.13 (s, 1H), 6.99 (s, 1H), 3.98 (s, 2H), 3.09 (s, 2H), 2.76 (s, 2H), 2.60-2.54 (m, 2H), 1.08 (t, J=7.5 Hz, 3H).

### Step 5: Methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate

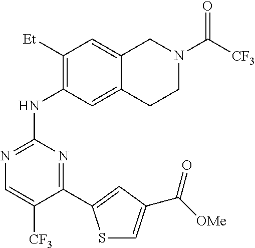 [SMILES: CCc1cc2c(cc1Nc1ncc(C(F)(F)F)c(-c3cc(C(=O)OC)cs3)n1)CCN(C(=O)C(F)(F)F)C2]

The title compound was prepared analogously to Example 1, step 6 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with 11-(6-((4-chloro-5-(trifluoromethyl)pyrimidin-2-yl)amino)-7-ethyl-3,4-dihydroisoquinolin-2(1H)-yl)-2,2,2-trifluoroethan-1-one. The title compound was isolated in 75% yield. m/z (ESI, +ve)=559.9 [M+H]⁺.

### Example 10: 5-(2-((7-cyclopropyl-1,2,3,4-tetrahydroisoquinolin-6-yl)amino)-5-(trifluoromethyl)pyrimidin-4-yl)thiophene-3-carboxamide

### Step 1: 1-(7-Cyclopropyl-6-nitro-3,4-dihydroisoquinolin-2(1H)-yl)-2,2,2-trifluoroethan-1-one

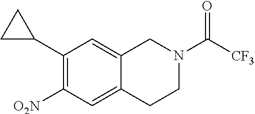 [SMILES: O=C(N1CCc2cc([N+](=O)[O-])c(C3CC3)cc2C1)C(F)(F)F]

The title compound was prepared analogously to Example 9, step 1, where ethylboronic acid was replaced with potassium cyclopropyltrifluoroborate. The title compound was isolated in 72% yield. m/z (ESI, +ve)=315.1 [M+H]⁺.

### Step 2: 1-(6-Amino-7-cyclopropyl-3,4-dihydroisoquinolin-2(1H)-yl)-2,2,2-trifluoroethan-1-one

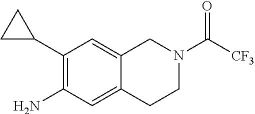 [SMILES: Nc1cc2c(cc1C1CC1)CN(C(=O)C(F)(F)F)CC2]

The title compound was prepared analogously to Example 9, step 3, where 1-(7-ethyl-6-nitro-1,2,3,4-tetrahydroisoquinolin-2-yl)-2,2,2-trifluoroethan-1-one was replaced with 1-(7-cyclopropyl-6-nitro-3,4-dihydroisoquinolin-2(1H)-yl)-2,2,2-trifluoroethan-1-one. The title compound was isolated in 79% yield. m/z (ESI, +ve)=285.3 [M+H]⁺.

### Step 3: 1-(6-((4-chloro-5-(trifluoromethyl)pyrimidin-2-yl)amino)-7-cyclopropyl-3,4-dihydroisoquinolin-2(1H)-yl)-2,2,2-trifluoroethan-1-one

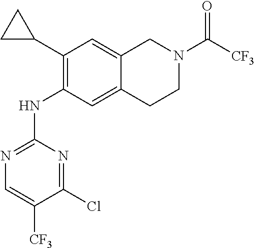 [SMILES: O=C(N1CCc2cc(Nc3ncc(C(F)(F)F)c(Cl)n3)c(C3CC3)cc2C1)C(F)(F)F]

The title compound was prepared analogously to example 9, step 4, where 1-(6-amino-7-ethyl-3,4-dihydroisoquinolin-2(1H)-yl)-2,2,2-trifluoroethan-1-one was replaced with 1-(6-amino-7-cyclopropyl-3,4-dihydroisoquinolin-2(1H)-yl)-2,2,2-trifluoroethan-1-one. The title compound was isolated in 33% yield. m/z (ESI, +ve)=465.2 [M+H]⁺.

### Step 4: Methyl 5-(2-((7-cyclopropyl-2-(2,2,2-trifluoroacetyl)-1,2,3,4-tetrahydroisoquinolin-6-yl)amino)-5-(trifluoromethyl)pyrimidin-4-yl)thiophene-3-carboxylate

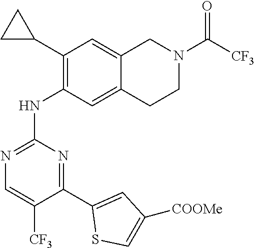 [SMILES: COC(=O)c1csc(-c2nc(Nc3cc4c(cc3C3CC3)CN(C(=O)C(F)(F)F)CC4)ncc2C(F)(F)F)c1]

The title compound was prepared analogously to Example 1, step 6, where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with 1-(6-((4-chloro-5-(trifluoromethyl)pyrimidin-2-yl)amino)-7-cyclopropyl-3,4-dihydroisoquinolin-2(1H)-yl)-2,2,2-trifluoroethan-1-one. The title compound was isolated in 66% yield. m/z (ESI, +ve)=571.9 [M+H]⁺.

### Step 5

The title compound was prepared analogously to Example 1 where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with methyl 5-(2-((7-cyclopropyl-2-(2,2,2-trifluoroacetyl)-1,2,3,4-tetrahydroisoquinolin-6-yl)amino)-5-(trifluoromethyl)pyrimidin-4-yl)thiophene-3-carboxylate. The title compound was isolated in 27% yield. m/z (ESI, +ve)=460.2 [M+H]⁺. ¹H NMR (400 MHz, DMSO-d₆) δ 9.78 (s, 1H), 8.75 (s, 1H), 8.41 (s, 1H), 8.02 (s, 1H), 7.96 (s, 1H), 7.39 (s, 1H), 7.34 (s, 1H), 6.87 (s, 1H), 4.22 (s, 2H), 3.38 (t, J=6.3 Hz, 2H), 2.97 (t, J=6.3 Hz, 2H), 2.09-1.92 (m, 1H), 0.95-0.80 (m, 2H), 0.67-0.52 (m, 2H).

### Example 11: 5-(2-((7-chloro-2-(1-methylazetidin-3-yl)-1,2,3,4-tetrahydroisoquinolin-6-yl)amino)-5-(trifluoromethyl)pyrimidin-4-yl)thiophene-3-carboxamide

To a stirred solution of 1-methylazetidin-3-one hydrochloride (0.17 mmol) and 5-(2-((7-chloro-1,2,3,4-tetrahydroisoquinolin-6-yl)amino)-5-(trifluoromethyl)pyrimidin-4-yl)thiophene-3-carboxamide (0.14 mmol) in dichloromethane (1.4 mL), triethylamine (0.20 mL) and NaBH(OAc)₃ (0.69 mmol) were added in one portion. The reaction mixture was stirred at room temperature overnight and quenched with water. The organic layer was separated and dried over sodium sulfate and concentrated to dryness. The crude product was purified by preparative HPLC to afford the title compound in 5% yield. m/z (ESI, +ve)=523.15 [M+H]⁺. ¹H NMR (400 MHz, DMSO-d₆) δ 9.82 (s, 1H), 8.77 (s, 1H), 8.42 (d, J=1.2 Hz, 1H), 8.02 (s, 1H), 7.96 (s, 1H), 7.37 (d, J=6.0 Hz, 2H), 7.27 (s, 1H), 3.47 (t, J=6.6 Hz, 2H), 3.43 (s, 2H), 3.02 (p, J=6.5 Hz, 1H), 2.85 (t, J=6.7 Hz, 2H), 2.80 (t, J=5.9 Hz, 2H), 2.25 (s, 3H).

### Example 12: 5-(2-((6-chloro-2-(1-methylazetidin-3-yl)isoindolin-5-yl)amino)-5-(trifluoromethyl)pyrimidin-4-yl)thiophene-3-carboxamide

5-(2-((6-chloroisoindolin-5-yl)amino)-5-(trifluoromethyl)pyrimidin-4-yl)thiophene-3-carboxamide (0.21 mmol), 1-methylazetidin-3-one hydrochloride (0.42 mmol) and sodium cyanoborohydride (0.42 mmol) were dissolved in methanol (2 mL). The reaction was stirred at 50° C. for 3 hours and purified by silica gel chromatography (dichloromethane with 10-40% of a 0.6M methanolic solution of ammonia). The product was dried and a second purification was carried out by eluting the dissolved compound through a NH2-silica cartridge using methanol as solvent. The title compound was isolated in 32% yield. m/z (ESI, +ve)=509.14 [M+H]⁺. ¹H NMR (400 MHz, DMSO-d₆) δ 9.89 (s, 1H), 8.76 (s, 1H), 8.42 (d, J=1.2 Hz, 1H), 8.01 (s, 1H), 7.96 (s, 1H), 7.46 (s, 1H), 7.44 (s, 1H), 7.39 (s, 1H), 3.83 (s, 4H), 3.45-3.38 (m, 3H), 3.05-2.94 (m, 2H), 2.26 (s, 3H).

### Example 13: 5-(2-((7-ethyl-2-methyl-1,2,3,4-tetrahydroisoquinolin-6-yl)amino)-5-(trifluoromethyl)pyrimidin-4-yl)thiophene-3-carboxamide

### Step 1: Methyl 5-(2-((7-Ethyl-2-methyl-1,2,3,4-tetrahydroisoquinolin-6-yl)amino)-5-(trifluoromethyl)pyrimidin-4-yl)thiophene-3-carboxylate

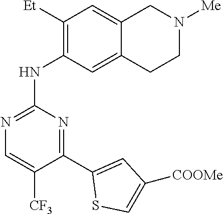 [SMILES: CCc1cc2c(cc1Nc1ncc(C(F)(F)F)c(-c3cc(C(=O)OC)cs3)n1)CCN(C)C2]

Methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate (0.69 mmol) was dissolved in THF (8 mL) and a 0.3M aqueous solution of LiOH (3 mL) was added dropwise. The reaction mixture was stirred at room temperature for 1 hour followed by the addition of acetic acid (0.12 mL) and a 37% aqueous solution of formaldehyde (0.072 mL). After 10 minutes, sodium cyanoborohydride (1.39 mmol) was added and one hour later all volatiles were removed under reduced pressure to afford a solid that was taken up in dichloromethane and water. The organic layer was separated, dried over sodium sulfate, filtered, and concentrated to afford the title compound in 84% yield. m/z (ESI, +ve)=477.4 [M+H]⁺

### Step 2: 5-(2-((7-Ethyl-2-methyl-1,2,3,4-tetrahydroisoquinolin-6-yl)amino)-5-(trifluoromethyl)pyrimidin-4-yl)thiophene-3-carboxylic acid

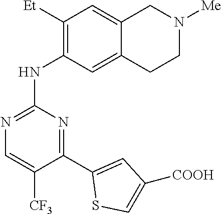 [SMILES: CCc1cc2c(cc1Nc1ncc(C(F)(F)F)c(-c3cc(C(=O)O)cs3)n1)CCN(C)C2]

Methyl 5-(2-((7-Ethyl-2-methyl-1,2,3,4-tetrahydroisoquinolin-6-yl)amino)-5-(trifluoromethyl)pyrimidin-4-yl)thiophene-3-carboxylate (0.58 mmol) was dissolved in THF (5.6 mL) and a 1M aqueous solution of LiOH (1.4 mL) was added. The reaction was stirred at room temperature overnight and the pH of the mixture was decreased to 4 by the addition of a 1M aqueous solution of HCl. The solvents were removed under reduced pressure affording the title compound in 96% yield. This material was used in the next step without further purification. m/z (ESI, +ve)=463.3 [M+H]⁺.

### Step 3

5-(2-((7-ethyl-2-methyl-1,2,3,4-tetrahydroisoquinolin-6-yl)amino)-5-(trifluoromethyl)pyrimidin-4-yl)thiophene-3-carboxylic acid hydrochloride (0.56 mmol) was dissolved in DMF (6.9 mL) and triethylamine (0.23 mL), a 0.4 N solution of ammonia in 1,4-dioxane (4.2 mL) and HATU (1.68 mmol) were added. The reaction mixture was stirred at room temperature for 1 hour, the volatiles were removed under reduced pressure and the residue was taken up in water and filtered. The crude material was purified by silica gel chromatography (dichloromethane with 10-50% of a 0.6M methanolic solution of ammonia) to afford the title compound in 52% yield. m/z (ESI, +ve)=462.1 [M+H]⁺. ¹H NMR (400 MHz, DMSO-d₆) δ 9.67 (s, 1H), 8.71 (s, 1H), 8.39 (s, 1H), 8.00 (s, 1H), 7.94 (s, 1H), 7.37 (s, 1H), 7.16 (s, 1H), 6.98 (s, 1H), 3.69 (s, 2H), 2.92-2.75 (m, 4H), 2.56 (q, J=8.5, 8.0 Hz, 2H), 1.08 (t, J=7.5 Hz, 3H).

### Example 14: 5-(2-((7-cyclopropyl-2-methyl-1,2,3,4-tetrahydroisoquinolin-6-yl)amino)-5-(trifluoromethyl)pyrimidin-4-yl)thiophene-3-carboxamide

### Step 1: Methyl 5-(2-((7-cyclopropyl-2-methyl-1,2,3,4-tetrahydroisoquinolin-6-yl)amino)-5-(trifluoromethyl)pyrimidin-4-yl)thiophene-3-carboxylate

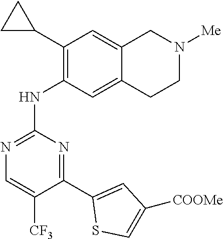 [SMILES: COC(=O)c1csc(-c2nc(Nc3cc4c(cc3C3CC3)CN(C)CC4)ncc2C(F)(F)F)c1]

The title compound was prepared analogously to Example 13, step 1, where methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with methyl 5-(2-((7-cyclopropyl-2-(2,2,2-trifluoroacetyl)-1,2,3,4-tetrahydroisoquinolin-6-yl)amino)-5-(trifluoromethyl)pyrimidin-4-yl)thiophene-3-carboxylate. The title compound was isolated in 99% yield. m/z (ESI, +ve)=489.4 [M+H]⁺.

### Step 2: 5-(2-((7-cyclopropyl-2-methyl-1,2,3,4-tetrahydroisoquinolin-6-yl)amino)-5-(trifluoromethyl)pyrimidin-4-yl)thiophene-3-carboxylic acid

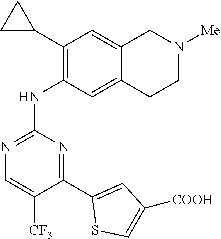 [SMILES: CN1CCc2cc(Nc3ncc(C(F)(F)F)c(-c4cc(C(=O)O)cs4)n3)c(C3CC3)cc2C1]

The title compound was prepared analogously to Example 13, step 2, where methyl 5-(2-((7-ethyl-2-methyl-1,2,3,4-tetrahydroisoquinolin-6-yl)amino)-5-(trifluoromethyl)pyrimidin-4-yl)thiophene-3-carboxylate was replaced with methyl 5-(2-((7-cyclopropyl-2-methyl-1,2,3,4-tetrahydroisoquinolin-6-yl)amino)-5-(trifluoromethyl)pyrimidin-4-yl)thiophene-3-carboxylate. The title compound was isolated in 98% yield. m/z (ESI, +ve)=475.3 [M+H]⁺.

### Step 3

A solution of 5-(2-((7-cyclopropyl-2-methyl-1,2,3,4-tetrahydroisoquinolin-6-yl)amino)-5-(trifluoromethyl)pyrimidin-4-yl)thiophene-3-carboxylic acid hydrochloride (0.36 mmol), triethylamine (0.15 mL) in DMF (4 mL) was treated with a 0.4 N solution of ammonia in 1,4-dioxane (2.7 mL) and HATU (1.08 mmol) was stirred at room temperature for 1 hour. The reaction mixture was concentrated to dryness and the solid residue was suspended in water and filtered. Purification by preparative HPLC afforded the title compound in 15% yield. m/z (ESI, +ve)=474.2 [M+H]⁺. ¹H NMR (400 MHz, DMSO-d₆) δ 9.64 (s, 1H), 8.72 (s, 1H), 8.41 (s, 1H), 8.29 (s, 2H), 8.01 (s, 1H), 7.96 (s, 1H), 7.38 (s, 1H), 7.18 (s, 1H), 6.67 (s, 1H), 3.44 (s, 2H), 2.79 (t, J=5.8 Hz, 2H), 2.58 (t, J=5.9 Hz, 2H), 2.33 (s, 3H), 2.00-1.86 (m, 1H), 0.84-0.76 (m, 2H), 0.59-0.51 (m, 2H).

### Example 15: N-(4-(4-aminothiophen-2-yl)-5-(trifluoromethyl)pyrimidin-2-yl)-7-chloro-1,2,3,4-tetrahydroisoquinolin-6-amine

### Step 1: tert-Butyl (5-(2-((7-chloro-2-(2,2,2-trifluoroacetyl)-1,2,3,4-tetrahydroisoquinolin-6-yl)amino)-5-(trifluoromethyl)pyrimidin-4-yl)thiophen-3-yl)carbamate

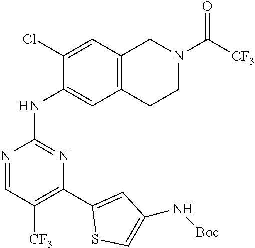 [SMILES: CC(C)(C)OC(=O)Nc1csc(-c2nc(Nc3cc4c(cc3Cl)CN(C(=O)C(F)(F)F)CC4)ncc2C(F)(F)F)c1]

The title compound was prepared analogously to Example 4, step 2, where 2-bromo-6,7-dihydrothieno[3,2-C]pyridin-4(5H)-one was replaced with 2-bromo-4-(N-tert-butyloxycarbonylamino)thiophene. The title compound was isolated in 28% yield. m/z (ESI, +ve)=623.3 [M+H]⁺.

### Step 2

tert-Butyl-(5-(2-((7-chloro-2-(2,2,2-trifluoroacetyl)-1,2,3,4-tetrahydroisoquinolin-6-yl)amino)-5-(trifluoromethyl)pyrimidin-4-yl)thiophen-3-yl)carbamate (0.038 mmol) was dissolved in a 2M solution of HCl in diethyl ether (2 mL) and the reaction was stirred at room temperature for 2 hours. After evaporation of volatiles, the residue was redissolved in a 7N solution of ammonia in methanol (0.55 mL) and the reaction was stirred for 16 hours. The solvents were removed under reduced pressure and the crude material was purified by HPLC to afford the title compound in 49% yield. m/z (ESI, +ve)=424.0 [M+H]⁺. ¹H NMR (400 MHz, DMSO-d₆) δ 9.59 (s, 1H), 8.69 (s, 1H), 8.18 (s, 1H), 7.42 (s, 1H), 7.28 (d, J=6.0 Hz, 2H), 6.37 (d, J=1.4 Hz, 1H), 5.06 (s, 2H), 3.97 (s, 2H), 3.08 (s, 2H), 2.78 (s, 2H).

### Example 16: N-(5-(2-((7-chloro-1,2,3,4-tetrahydroisoquinolin-6-yl)amino)-5-(trifluoromethyl)pyrimidin-4-yl)thiophen-3-yl)methanesulfonamide

### Step 1: tert-butyl (5-(2-((7-chloro-2-(2,2,2-trifluoroacetyl)-1,2,3,4-tetrahydroisoquinolin-6-yl)amino)-5-(trifluoromethyl)pyrimidin-4-yl)thiophen-3-yl)carbamate

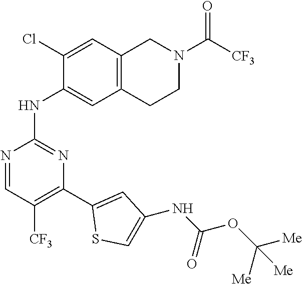 [SMILES: CC(C)(C)OC(=O)Nc1csc(-c2nc(Nc3cc4c(cc3Cl)CN(C(=O)C(F)(F)F)CC4)ncc2C(F)(F)F)c1]

The title compound was prepared analogously to Example 4, step 2, where 2-bromo-6,7-dihydrothieno[3,2-C]pyridin-4(5H)-one was replaced with 2-bromo-4-(N-tert-butyloxycarbonylamino)thiophene. The title compound was isolated in 92% yield. m/z (ESI, +ve)=622.2 [M+H]⁺

### Step 2: 1-(6-((4-(4-aminothiophen-2-yl)-5-(trifluoromethyl)pyrimidin-2-yl)amino)-7-chloro-3,4-dihydroisoquinolin-2(1H)-yl)-2,2,2-trifluoroethan-1-one

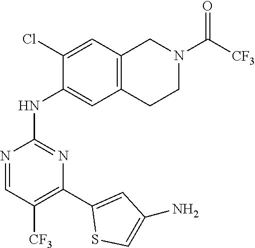 [SMILES: Nc1csc(-c2nc(Nc3cc4c(cc3Cl)CN(C(=O)C(F)(F)F)CC4)ncc2C(F)(F)F)c1]

The title compound was prepared analogously to Example 5 where tert-butyl 3-(6-((4-(4-carbamoylthiophen-2-yl)-5-(trifluoromethyl)pyrimidin-2-yl)amino)-7-chloro-3,4-dihydroisoquinolin-2(1H)-yl)azetidine-1-carboxylate was replaced with tert-butyl (5-(2-((7-chloro-2-(2,2,2-trifluoroacetyl)-1,2,3,4-tetrahydroisoquinolin-6-yl)amino)-5-(trifluoromethyl)pyrimidin-4-yl)thiophen-3-yl)carbamate. The title compound was isolated in 92% yield. m/z (ESI, +ve)=522.2 [M+H]⁺.

### Step 3

To the stirred suspension of N-(4-(4-aminothiophen-2-yl)-5-(trifluoromethyl)pyrimidin-2-yl)-7-chloro-1,2,3,4-tetrahydroisoquinolin-6-amine hydrochloride (0.096 mmol) in dichloromethane (1.0 mL), triethylamine (0.028 mL) and methanesulfonyl chloride (0.007 mL) were added. The reaction mixture was stirred at room temperature overnight and the volatiles were removed under reduced pressure to afford a residue that was treated with a 7N solution of ammonia in methanol (1.4 mL). The resulting reaction was stirred at 50° C. for 2 hours, concentrated to dryness and purified by silica gel chromatography (dichloromethane with a 10% methanolic solution of ammonia) to afford the title compound in 52% yield. m/z (ESI, +ve)=501.8 [M+H]⁺. ¹H NMR (400 MHz, DMSO-d₆) δ 9.75 (s, 1H), 8.75 (s, 1H), 7.63 (s, 1H), 7.33 (d, J=1.5 Hz, 2H), 7.22 (s, 1H), 3.86 (s, 2H), 2.97 (d, J=10.2 Hz, 5H), 2.71 (d, J=5.9 Hz, 3H).

### Example 17: 5-(2-((6-Chloro-2-methylisoindolin-5-yl)amino)-5-(trifluoromethyl)pyrimidin-4-yl)thiophene-3-carboxamide

5-(2-((6-chloroisoindolin-5-yl)amino)-5-(trifluoromethyl)pyrimidin-4-yl)thiophene-3-carboxamide (0.16 mmol) and formaldehyde (37 wt. % in water, 0.02 mL) were dissolved in methanol (2.4 mL) and stirred at room temperature for 10 minutes. Sodium cyanoborohydride (0.33 mmol) was added and the reaction was stirred for another hour. After evaporation of volatiles under reduced pressure, the crude material was purified by HPLC to afford the title compound in 12% yield. m/z (ESI, +ve)=454.4 [M+H]⁺. ¹H NMR (400 MHz, DMSO-d₆) δ 9.90 (s, 1H), 8.77 (s, 1H), 8.41 (d, J=1.2 Hz, 1H), 8.14 (s, 1H), 8.02 (s, 1H), 7.97 (s, 1H), 7.47 (s, 1H), 7.46 (s, 1H), 7.40 (s, 1H), 3.91 (s, 4H), 2.55 (s, 3H).

### Example 18: 7-Chloro-N-(4-(4-(oxazol-2-yl)thiophen-2-yl)-5-(trifluoromethyl)pyrimidin-2-yl)-1,2,3,4-tetrahydroisoquinolin-6-amine

### Step 1: 2-(5-Bromothiophen-3-yl)oxazole

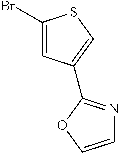 [SMILES: Brc1cc(-c2ncco2)cs1]

2-(5-Bromothiophen-3-yl)-4,5-dihydro-1,3-oxazole (1.08 mmol) and 2,3-dichloro-5,6-dicyano-1,4-benzoquinone (1.62 mmol) in 1,4-dioxane (5.0 mL) were stirred at 100° C. for 2 hours. The reaction mixture was concentrated and purified by silica gel chromatography (0-100% dichloromethane in hexanes) to afford the title compound in 36% yield. m/z (ESI, +ve)=232.0 [M+H]⁺.

### Step 2: 1-(7-Chloro-6-((4-(4-(oxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one

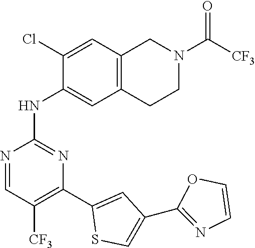 [SMILES: O=C(N1CCc2cc(Nc3ncc(C(F)(F)F)c(-c4cc(-c5ncco5)cs4)n3)c(Cl)cc2C1)C(F)(F)F]

The title compound was prepared analogously to Example 4, step 2 where 2-bromo-6,7-dihydrothieno[3,2-C]pyridin-4(5H)-one was replaced with 2-(5-bromothiophen-3-yl)oxazole. The title compound was isolated in 20% yield. m/z (ESI, +ve)=574.8 [M+H]⁺.

### Step 3

The title compound was prepared analogously to Example 1 where methyl-5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 1-(7-chloro-6-((4-(4-(oxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one. The title compound was isolated in 56% yield. m/z (ESI, +ve)=478.0 [M+H]⁺. ¹H NMR (400 MHz, DMSO-d₆) δ 9.87 (s, 1H), 8.80 (s, 1H), 8.48 (d, J=1.2 Hz, 1H), 8.20 (d, J=0.8 Hz, 1H), 8.12 (s, 1H), 7.36 (d, J=0.8 Hz, 1H), 7.32 (s, 1H), 7.23 (s, 1H), 3.85 (s, 2H), 2.95 (t, J=5.8 Hz, 2H), 2.70 (m, 3H).

### Example 19: 5-(2-((6-Cyclopropyl-2-methylisoindolin-5-yl)amino)-5-(trifluoromethyl)pyrimidin-4-yl)thiophene-3-carboxamide

### Step 1: 1-(5-Cyclopropylisoindolin-2-yl)-2,2,2-trifluoroethan-1-one

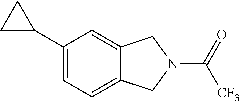 [SMILES: O=C(N1Cc2ccc(C3CC3)cc2C1)C(F)(F)F]

The title compound was prepared analogously to Example 9, step 1, where 1-(7-chloro-6-nitro-1,2,3,4-tetrahydroisoquinolin-2-yl)-2,2,2-trifluoroethan-1-one and ethylboronic acid were replaced with 1-(5-bromoisoindolin-2-yl)-2,2,2-trifluoroethan-1-one and cyclopropylboronic acid. The title compound was isolated in 52% yield. m/z (ESI, +ve)=256.1 [M+H]⁺.

### Step 2: 1-(5-Cyclopropyl-6-nitroisoindolin-2-yl)-2,2,2-trifluoroethan-1-one

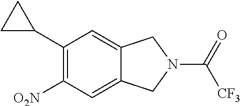 [SMILES: O=C(N1Cc2cc(C3CC3)c([N+](=O)[O-])cc2C1)C(F)(F)F]

The title compound was prepared analogously to Example 1, step 3, where 1-(7-chloro-1,2,3,4-tetrahydroisoquinolin-2-yl)-2,2,2-trifluoroethan-1-one was replaced with 1-(5-cyclopropylisoindolin-2-yl)-2,2,2-trifluoroethan-1-one. The title compound was isolated in 31% yield. m/z (ESI, +ve)=301.2 [M+H]⁺.

### Step 3: 1-(5-Amino-6-cyclopropylisoindolin-2-yl)-2,2,2-trifluoroethan-1-one

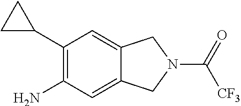 [SMILES: Nc1cc2c(cc1C1CC1)CN(C(=O)C(F)(F)F)C2]

A solution of 1-(5-cyclopropyl-6-nitroisoindolin-2-yl)-2,2,2-trifluoroethan-1-one (2.40 mmol) in methanol (29 mL) was hydrogenated in the presence of 10% palladium on carbon (0.72 mmol) for two hours. The reaction was filtered through celite and concentrated to afford the title compound in 93% yield. m/z (ESI, +ve)=271.2 [M+H]⁺.

### Step 4: 1-(5-((4-Chloro-5-(trifluoromethyl)pyrimidin-2-yl)amino)-6-cyclopropylisoindolin-2-yl)-2,2,2-trifluoroethan-1-one

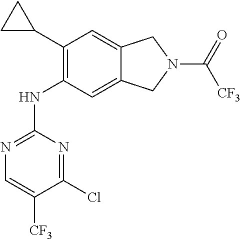 [SMILES: O=C(N1Cc2cc(Nc3ncc(C(F)(F)F)c(Cl)n3)c(C3CC3)cc2C1)C(F)(F)F]

The title compound was prepared analogously to Example 1, step 5, where 1-(6-amino-7-chloro-1,2,3,4-tetrahydroisoquinolin-2-yl)-2,2,2-trifluoroethan-1-one was replaced with 1-(5-amino-6-cyclopropylisoindolin-2-yl)-2,2,2-trifluoroethan-1-one. The title compound was isolated in 34% yield. m/z (ESI, +ve)=451.1 [M+H]⁺.

### Step 5: Methyl 5-(2-((6-cyclopropyl-2-(2,2,2-trifluoroacetyl)isoindolin-5-yl)amino)-5-(trifluoromethyl)pyrimidin-4-yl)thiophene-3-carboxylate

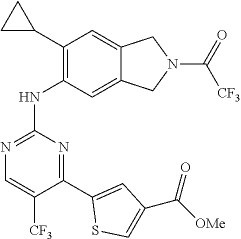 [SMILES: COC(=O)c1csc(-c2nc(Nc3cc4c(cc3C3CC3)CN(C(=O)C(F)(F)F)C4)ncc2C(F)(F)F)c1]

The title compound was prepared analogously to Example 1, step 6, where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with 1-(5-((4-chloro-5-(trifluoromethyl)pyrimidin-2-yl)amino)-6-cyclopropylisoindolin-2-yl)-2,2,2-trifluoroethan-1-one. The title compound was isolated in 73% yield. m/z (ESI, +ve)=557.3 [M+H]⁺.

### Step 6

The title compound was prepared analogously to Example 13 where 5-(2-((7-ethyl-2-methyl-1,2,3,4-tetrahydroisoquinolin-6-yl)amino)-5-(trifluoromethyl)pyrimidin-4-yl)thiophene-3-carboxylic acid hydrochloride was replaced with 5-(2-((6-cyclopropyl-2-methylisoindolin-5-yl)amino)-5-(trifluoromethyl)pyrimidin-4-yl)thiophene-3-carboxylic acid. The title compound was isolated in 49% yield. m/z (ESI, +ve)=460.2 [M+H]⁺. ¹H NMR (400 MHz, DMSO-d₆) δ 9.88 (s, 1H), 8.75 (s, 1H), 8.40 (d, J=1.2 Hz, 1H), 8.02 (s, 1H), 7.97 (s, 1H), 7.44 (s, 1H), 7.42 (s, 1H), 7.04 (s, 1H), 4.48 (s, 4H), 2.97 (s, 3H), 2.02 (m, 1H), 0.96-0.81 (m, 2H), 0.66-0.54 (m, 2H).

### Example 20: 5-(2-((6-Ethyl-2-methylisoindolin-5-yl)amino)-5-(trifluoromethyl)pyrimidin-4-yl)thiophene-3-carboxamide

### Step 1: 1-(5-Bromoisoindolin-2-yl)-2,2,2-trifluoroethan-1-one

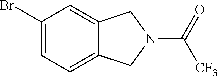 [SMILES: O=C(N1Cc2ccc(Br)cc2C1)C(F)(F)F]

The title compound was prepared analogously to Example 7, step 1 where 5-chloroisoindoline was replaced with 5-bromoisoindoline. The title compound was isolated in 96% yield. ¹H NMR (400 MHz, DMSO-d₆) δ 7.69-7.59 (m, 1H), 7.53 (dd, J=8.2, 1.9 Hz, 1H), 7.41-7.30 (m, 1H), 5.01 (d, J=12.0 Hz, 2H), 4.81 (d, J=12.8 Hz, 2H).

### Step 2: 1-(5-Ethylisoindolin-2-yl)-2,2,2-trifluoroethan-1-one

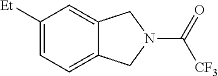 [SMILES: CCc1ccc2c(c1)CN(C(=O)C(F)(F)F)C2]

The title compound was prepared analogously to Example 9, step 1, where 1-(7-chloro-6-nitro-1,2,3,4-tetrahydroisoquinolin-2-yl)-2,2,2-trifluoroethan-1-one was replaced with 1-(5-bromoisoindolin-2-yl)-2,2,2-trifluoroethan-1-one. The title compound was isolated in 97% yield. m/z (ESI, +ve)=244.1 [M+H]⁺.

### Step 3: 1-(5-Ethyl-6-nitroisoindolin-2-yl)-2,2,2-trifluoroethan-1-one

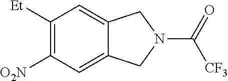 [SMILES: CCc1cc2c(cc1[N+](=O)[O-])CN(C(=O)C(F)(F)F)C2]

The title compound was prepared analogously to Example 7, step 2, where 1-(5-chloro-2,3-dihydro-1H-isoindol-2-yl)-2,2,2-trifluoroethan-1-one was replaced with 1-(5-Ethylisoindolin-2-yl)-2,2,2-trifluoroethan-1-one. The title compound was isolated in 41% yield. m/z (ESI, +ve)=289.2 [M+H]⁺.

### Step 4: 1-(5-Amino-6-ethylisoindolin-2-yl)-2,2,2-trifluoroethan-1-one

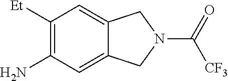 [SMILES: CCc1cc2c(cc1N)CN(C(=O)C(F)(F)F)C2]

A solution of 1-(5-ethyl-6-nitro-2,3-dihydro-1H-isoindol-2-yl)-2,2,2-trifluoroethan-1-one (4.10 mmol) in methanol (47 mL) was hydrogenated in the presence of 10% palladium on carbon (130 mg) for 2 hours. The reaction was filtered through celite and the solvents were removed under reduced pressure to afford the title compound in 93% yield. m/z (ESI, +ve)=259.1 [M+H]⁺.

### Step 5: 1-(5-((4-Chloro-5-(trifluoromethyl)pyrimidin-2-yl)amino)-6-ethylisoindolin-2-yl)-2,2,2-trifluoroethan-1-one

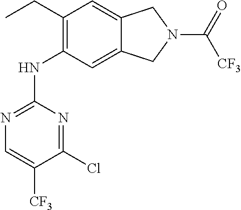 [SMILES: CCc1cc2c(cc1Nc1ncc(C(F)(F)F)c(Cl)n1)CN(C(=O)C(F)(F)F)C2]

The title compound was prepared analogously to Example 7, step 4, where 1-(5-amino-6-chloro-2,3-dihydro-1H-isoindol-2-yl)-2,2,2-trifluoroethan-1-one was replaced with 1-(5-amino-6-ethylisoindolin-2-yl)-2,2,2-trifluoroethan-1-one. The title compound was isolated in 23% yield. m/z (ESI, +ve)=439.1 [M+H]⁺.

### Step 6: Methyl 5-(2-((6-ethyl-2-(2,2,2-trifluoroacetyl)isoindolin-5-yl)amino)-5-(trifluoromethyl)pyrimidin-4-yl)thiophene-3-carboxylate

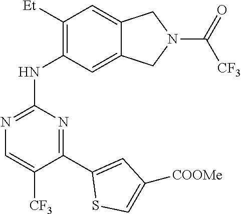 [SMILES: CCc1cc2c(cc1Nc1ncc(C(F)(F)F)c(-c3cc(C(=O)OC)cs3)n1)CN(C(=O)C(F)(F)F)C2]

The title compound was prepared analogously to Example 1, step 6, where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with 1-(5-((4-chloro-5-(trifluoromethyl)pyrimidin-2-yl)amino)-6-ethylisoindolin-2-yl)-2,2,2-trifluoroethan-1-one. The title compound was isolated in 55% yield. m/z (ESI, +ve)=545.2 [M+H]⁺.

### Step 7: Methyl 5-(2-((6-ethyl-2-methylisoindolin-5-yl)amino)-5-(trifluoromethyl)pyrimidin-4-yl)thiophene-3-carboxylate

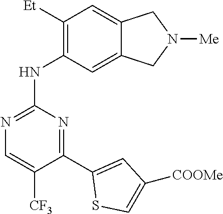 [SMILES: CCc1cc2c(cc1Nc1ncc(C(F)(F)F)c(-c3cc(C(=O)OC)cs3)n1)CN(C)C2]

The title compound was prepared analogously to Example 13, step 1 where methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with methyl 5-(2-((6-ethyl-2-(2,2,2-trifluoroacetyl)isoindolin-5-yl)amino)-5-(trifluoromethyl)pyrimidin-4-yl)thiophene-3-carboxylate. The title compound was isolated in 99% yield. m/z (ESI, +ve)=463.3 [M+H]⁺.

### Step 8: 5-(2-((6-Ethyl-2-methylisoindolin-5-yl)amino)-5-(trifluoromethyl)pyrimidin-4-yl)thiophene-3-carboxylic acid

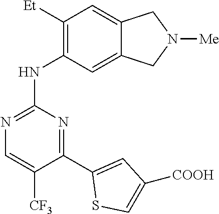 [SMILES: CCc1cc2c(cc1Nc1ncc(C(F)(F)F)c(-c3cc(C(=O)O)cs3)n1)CN(C)C2]

The title compound was prepared analogously to Example 2, step 1 where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with methyl 5-(2-((6-ethyl-2-methylisoindolin-5-yl)amino)-5-(trifluoromethyl)pyrimidin-4-yl)thiophene-3-carboxylate. The title compound was isolated in 99% yield. m/z (ESI, +ve)=449.3 [M+H]⁺.

### Step 9

The title compound was prepared analogously to Example 13 where 5-(2-((7-ethyl-2-methyl-1,2,3,4-tetrahydroisoquinolin-6-yl)amino)-5-(trifluoromethyl)pyrimidin-4-yl)thiophene-3-carboxylic acid hydrochloride was replaced with 5-(2-((6-ethyl-2-methylisoindolin-5-yl)amino)-5-(trifluoromethyl)pyrimidin-4-yl)thiophene-3-carboxylic acid. The title compound was isolated in 49% yield. m/z (ESI, +ve)=448.1 [M+H]⁺. ¹H NMR (400 MHz, DMSO-d₆) δ 9.69 (s, 1H), 8.71 (s, 1H), 8.40 (s, 1H), 8.23 (s, 1H), 8.00 (s, 1H), 7.96 (s, 1H), 7.39 (s, 1H), 7.21 (s, 1H), 7.16 (s, 1H), 3.83 (s, 4H), 2.59 (t, J=7.5 Hz, 2H), 1.09 (t, J=7.5 Hz, 3H).

### Example 21: 2-(2-((7-Chloro-1,2,3,4-tetrahydroisoquinolin-6-yl)amino)-5-(trifluoromethyl)pyrimidin-4-yl)-5-methyl-6,7-dihydrothieno[3,2-c]pyridin-4(5H)-one

### Step 1: 2-Bromo-5-methyl-6,7-dihydrothieno[3,2-c]pyridin-4(5H)-one

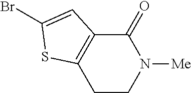 [SMILES: CN1CCc2sc(Br)cc2C1=O]

Cesium carbonate (2.15 mmol), 2-bromo-6,7-dihydrothieno[3,2-c]pyridin-4(5h)-one (0.43 mmol) and methyl iodide (0.14 mL) were suspended in DMF (2.0 mL) and the mixture stirred at 40° C. overnight. The volatiles were removed under reduced pressure, the residue was taken up in dichloromethane and water, the organic layer was separated, dried over sodium sulfate, filtered, and concentrated. Purification by silica gel chromatography (50% ethyl acetate in hexanes) afforded the title compound in 52% yield. m/z (ESI, +ve)=247.9 [M+H]⁺.

### Step 2: 2-(2-((7-Chloro-2-(2,2,2-trifluoroacetyl)-1,2,3,4-tetrahydroisoquinolin-6-yl)amino)-5-(trifluoromethyl)pyrimidin-4-yl)-5-methyl-6,7-dihydrothieno[3,2-c]pyridin-4(5H)-one

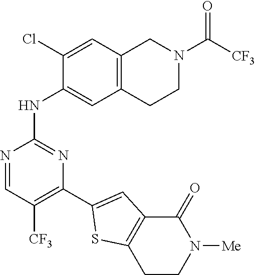 [SMILES: CN1CCc2sc(-c3nc(Nc4cc5c(cc4Cl)CN(C(=O)C(F)(F)F)CC5)ncc3C(F)(F)F)cc2C1=O]

The title compound was prepared analogously to Example 4, step 2, where 2-bromo-6,7-dihydrothieno[3,2-C]pyridin-4(5H)-one was replaced with 2-bromo-5-methyl-6,7-dihydrothieno[3,2-c]pyridin-4(5H)-one. The title compound was isolated in 62% yield. m/z (ESI, +ve)=588.4 [M+H]⁺.

### Step 3

The title compound was prepared analogously to Example 13 where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 2-(2-((7-chloro-2-(2,2,2-trifluoroacetyl)-1,2,3,4-tetrahydroisoquinolin-6-yl)amino)-5-(trifluoromethyl)pyrimidin-4-yl)-5-methyl-6,7-dihydrothieno[3,2-c]pyridin-4(5H)-one. The title compound was isolated in 36% yield. m/z (ESI, +ve)=493.9 [M+H]⁺. ¹H NMR (400 MHz, DMSO-d₆) δ 9.83 (s, 1H), 8.76 (s, 1H), 7.84 (s, 1H), 7.34 (s, 1H), 7.27 (s, 1H), 3.93 (s, 2H), 3.64 (t, J=6.8 Hz, 4H), 3.14 (t, J=6.8 Hz, 2H), 3.02 (s, 1H), 2.97 (s, 3H), 2.75 (s, 2H).

### Example 22: 2-(2-((7-chloro-1,2,3,4-tetrahydroisoquinolin-6-yl)amino)-5-(trifluoromethyl)pyrimidin-4-yl)-5-ethyl-6,7-dihydrothieno[3,2-c]pyridin-4(5H)-one

### Step 1: 2-bromo-5-ethyl-6,7-dihydrothieno[3,2-c]pyridin-4(5H)-one

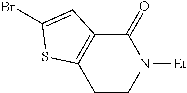 [SMILES: CCN1CCc2sc(Br)cc2C1=O]

The title compound was prepared analogously to Example 21, step1 where methyl iodide was replaced with ethyl iodide. The title compound was isolated in 45% yield. m/z (ESI, +ve)=260.0 [M+H]⁺

### Step 2: 2-(2-((7-chloro-2-(2,2,2-trifluoroacetyl)-1,2,3,4-tetrahydroisoquinolin-6-yl)amino)-5-(trifluoromethyl)pyrimidin-4-yl)-5-ethyl-6,7-dihydrothieno[3,2-c]pyridin-4(5H)-one

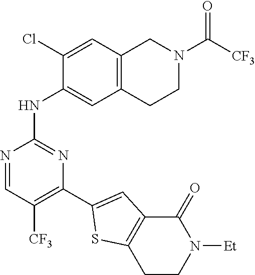 [SMILES: CCN1CCc2sc(-c3nc(Nc4cc5c(cc4Cl)CN(C(=O)C(F)(F)F)CC5)ncc3C(F)(F)F)cc2C1=O]

The title compound was prepared analogously to Example 4, step 2, where 2-bromo-6,7-dihydrothieno[3,2-C]pyridin-4(5H)-one was replaced with 2-bromo-5-ethyl-6,7-dihydrothieno[3,2-c]pyridin-4(5H)-one. The title compound was isolated in 65% yield. m/z (ESI, +ve)=604.1 [M+H]⁺.

### Step 3

The title compound was prepared analogously to Example 8 where 1-(7-chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one was replaced with 2-(2-((7-chloro-2-(2,2,2-trifluoroacetyl)-1,2,3,4-tetrahydroisoquinolin-6-yl)amino)-5-(trifluoromethyl)pyrimidin-4-yl)-5-ethyl-6,7-dihydrothieno[3,2-c]pyridin-4(5H)-one. The title compound was isolated in 77% yield. m/z (ESI, +ve)=508.1 [M+H]⁺. ¹H NMR (400 MHz, DMSO-d₆) δ 9.80 (s, 1H), 8.75 (s, 1H), 7.84 (s, 1H), 7.29 (s, 1H), 7.22 (s, 1H), 3.84 (s, 2H), 3.64 (t, J=6.8 Hz, 2H), 3.45 (q, J=7.1 Hz, 2H), 3.18-3.06 (m, 2H), 2.94 (t, J=5.8 Hz, 2H), 2.68 (t, J=5.9 Hz, 2H), 1.10 (t, J=7.1 Hz, 3H).

### Example 23: 2-(2-((7-Chloro-1,2,3,4-tetrahydroisoquinolin-6-yl)amino)-5-(trifluoromethyl)pyrimidin-4-yl)thieno[3,2-c]pyridin-4(5H)-one

### Step 1: 2-(2-((7-Chloro-2-(2,2,2-trifluoroacetyl)-1,2,3,4-tetrahydroisoquinolin-6-yl)amino)-5-(trifluoromethyl)pyrimidin-4-yl)thieno[3,2-c]pyridin-4(5H)-one

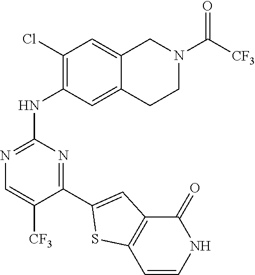 [SMILES: O=C(N1CCc2cc(Nc3ncc(C(F)(F)F)c(-c4cc5c(=O)[nH]ccc5s4)n3)c(Cl)cc2C1)C(F)(F)F]

The title compound was prepared analogously to Example 4, step 2, where 2-bromo-6,7-dihydrothieno[3,2-C]pyridin-4(5H)-one was replaced with 2-bromothieno[3,2-c]pyridin-4(5h)-one. The title compound was isolated in 39% yield. m/z (ESI, +ve)=574.2 [M+H]⁺.

### Step 2

The title compound was prepared analogously to Example 8 where 1-(7-chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one was replaced with 2-(2-((7-chloro-2-(2,2,2-trifluoroacetyl)-1,2,3,4-tetrahydroisoquinolin-6-yl)amino)-5-(trifluoromethyl)pyrimidin-4-yl)thieno[3,2-c]pyridin-4(5H)-one. The title compound was isolated in 25% yield. m/z (ESI, +ve)=478.2 [M+H]⁺. ¹H NMR (400 MHz, DMSO-d₆) δ 11.66 (s, 1H), 9.90 (s, 1H), 8.78 (s, 1H), 8.06 (s, 1H), 7.39 (d, J=11.5 Hz, 2H), 7.33 (s, 1H), 6.87 (d, J=7.0 Hz, 1H), 4.03 (s, 2H), 3.13 (s, 2H), 2.82 (s, 2H).

### Example 24: 2-(2-((7-Chloro-1,2,3,4-tetrahydroisoquinolin-6-yl)amino)-5-(trifluoromethyl)pyrimidin-4-yl)thieno[2,3-d]pyridazin-4(5H)-one

### Step 1: 2-(2-((7-chloro-2-(2,2,2-trifluoroacetyl)-1,2,3,4-tetrahydroisoquinolin-6-yl)amino)-5-(trifluoromethyl)pyrimidin-4-yl)thieno[2,3-d]pyridazin-4(5H)-one

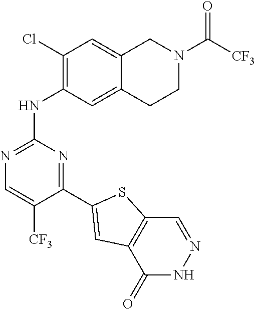 [SMILES: O=C(N1CCc2cc(Nc3ncc(C(F)(F)F)c(-c4cc5c(=O)[nH]ncc5s4)n3)c(Cl)cc2C1)C(F)(F)F]

The title compound was prepared analogously to Example 4, step 2, where ethyl 2-chlorooxazole-4-carboxylate was replaced with 2-bromothieno[2,3-D]pyridazine-4(5H)-one. The title compound was isolated in 48% yield. m/z (ESI, +ve)=575.2 [M+H]⁺.

### Step 2

The title compound was prepared analogously to Example 8 where 1-(7-chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one was replaced with 2-(2-((7-chloro-2-(2,2,2-trifluoroacetyl)-1,2,3,4-tetrahydroisoquinolin-6-yl)amino)-5-(trifluoromethyl)pyrimidin-4-yl)thieno[2,3-d]pyridazin-4(5H)-one. The title compound was isolated in 27% yield. m/z (ESI, +ve)=479.1 [M+H]⁺. ¹H NMR (400 MHz, DMSO-d₆) δ 8.83 (s, 1H), 8.59 (s, 1H), 8.31 (s, 1H), 8.07 (s, 1H), 7.45 (s, 1H), 7.39 (s, 1H), 4.13 (s, 2H), 3.22 (d, J=6.4 Hz, 2H), 2.91 (s, 2H).

### Example 25: 5-(2-((7-Chloro-1,2,3,4-tetrahydroisoquinolin-6-yl)amino)-5-(trifluoromethyl)pyrimidin-4-yl)thiophene-3-carbonitrile

### Step 1: 5-(2-((7-Chloro-2-(2,2,2-trifluoroacetyl)-1,2,3,4-tetrahydroisoquinolin-6-yl)amino)-5-(trifluoromethyl)pyrimidin-4-yl)thiophene-3-carbonitrile

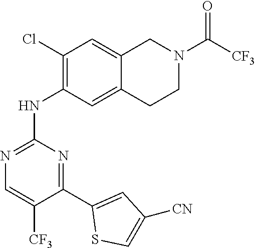 [SMILES: N#Cc1csc(-c2nc(Nc3cc4c(cc3Cl)CN(C(=O)C(F)(F)F)CC4)ncc2C(F)(F)F)c1]

The title compound was prepared analogously to Example 1, step 6, where 4-(methoxycarbonyl)thiophene-2-boronic acid pinacol ester was replaced with 5-(4,4,5,5-tetramethyl-1,3,2-dioxaborolan-2-yl)-3-thiophenecarbonitrile. The title compound was isolated in 9% yield. m/z (ESI, +ve)=532.1 [M+H]⁺.

### Step 2

The title compound was prepared analogously to Example 8, where 1-(7-chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one was replaced was replaced with 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carbonitrile. The title compound was isolated in 12% yield. m/z (ESI, +ve)=436.1 [M+H]⁺. ¹H NMR (400 MHz, DMSO-d₆) δ 9.97 (s, 1H), 8.84 (s, 1H), 8.82 (s, 1H), 7.91 (s, 1H), 7.28 (s, 1H), 7.22 (s, 1H), 3.84 (m, 2H), 2.94 (m, 2H), 2.67 (m, 2H).

### Example 26: 4-(4-Carbamoylthiophen-2-yl)-2-((7-chloro-1,2,3,4-tetrahydroisoquinolin-6-yl)amino)pyrimidine-5-carboxamide

### Step 1: Methyl 5-(5-carbamoyl-2-chloropyrimidin-4-yl)thiophene-3-carboxylate

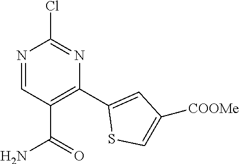 [SMILES: COC(=O)c1csc(-c2nc(Cl)ncc2C(N)=O)c1]

The title compound was prepared analogously to Example 1, step 6, where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one and 4-(methoxycarbonyl)thiophene-2-boronic acid pinacol ester were replaced with 2,4-dichloropyrimidine-5-carboxamide and 4-(methoxycarbonyl)thiophene-2-boronic acid pinacol ester. The title compound was isolated in 35% yield. m/z (ESI, +ve)=298.1 [M+H]⁺.

### Step 2: Methyl 5-(5-carbamoyl-2-((7-chloro-2-(2,2,2-trifluoroacetyl)-1,2,3,4-tetrahydroisoquinolin-6-yl)amino)pyrimidin-4-yl)thiophene-3-carboxylate

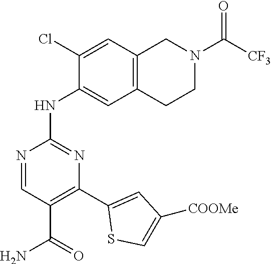 [SMILES: COC(=O)c1csc(-c2nc(Nc3cc4c(cc3Cl)CN(C(=O)C(F)(F)F)CC4)ncc2C(N)=O)c1]

A solution of methyl 5-(5-carbamoyl-2-chloropyrimidin-4-yl)thiophene-3-carboxylate (0.34 mmol), 1-(6-amino-7-chloro-1,2,3,4-tetrahydroisoquinolin-2-yl)-2,2,2-trifluoroethan-1-one (0.67 mmol) and (1S)-(+)-10-camphorsulfonic acid (0.672 mmol) in N-methylpyrrolidone (2.5 mL) was stirred in a sealed tube at 100° C. overnight. Additional (1S)-(+)-10-camphorsulfonic acid (0.67 mmol) was added and the reaction was continued at 100° C. for 24 hours. A third batch of (1S)-(+)-10-camphorsulfonic acid (0.67 mmol) was added and the reaction was continued at 100° C. for another 24 hours. The reaction was cooled down to room temperature and a saturated aqueous solution of NaHCO3 (50 mL) was added. The precipitate was collected by filtration and triturated with dichloromethane to afford the title compound in 75% yield. m/z (ESI, +ve)=540.2 [M+H]⁺.

### Step 3: Methyl 5-(5-carbamoyl-2-((7-chloro-1,2,3,4-tetrahydroisoquinolin-6-yl)amino)pyrimidin-4-yl)thiophene-3-carboxylate

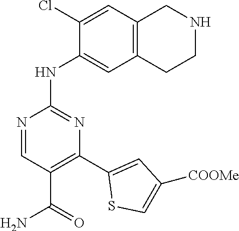 [SMILES: COC(=O)c1csc(-c2nc(Nc3cc4c(cc3Cl)CNCC4)ncc2C(N)=O)c1]

The title compound was prepared analogously to Example 8 where 1-(7-chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one was replaced with methyl 5-(5-carbamoyl-2-((7-chloro-2-(2,2,2-trifluoroacetyl)-1,2,3,4-tetrahydroisoquinolin-6-yl)amino)pyrimidin-4-yl)thiophene-3-carboxylate. The title compound was isolated in 99% yield. m/z (ESI, +ve)=444.1 [M+H]⁺.

### Step 4: tert-Butyl 6-((5-carbamoyl-4-(4-(methoxycarbonyl)thiophen-2-yl)pyrimidin-2-yl)amino)-7-chloro-3,4-dihydroisoquinoline-2(1H)-carboxylate

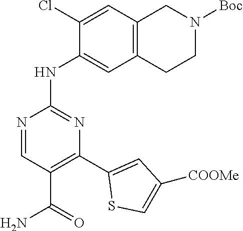 [SMILES: COC(=O)c1csc(-c2nc(Nc3cc4c(cc3Cl)CN(C(=O)OC(C)(C)C)CC4)ncc2C(N)=O)c1]

The title compound was prepared analogously to Example 2, step 2, where 5-(2-((7-chloro-1,2,3,4-tetrahydroisoquinolin-6-yl)amino)-5-(trifluoromethyl)pyrimidin-4-yl)thiophene-3-carboxylic acid was replaced with methyl 5-(5-carbamoyl-2-((7-chloro-1,2,3,4-tetrahydroisoquinolin-6-yl)amino)pyrimidin-4-yl)thiophene-3-carboxylate. The title compound was isolated in 99% yield. m/z (ESI, +ve)=544.2 [M+H]⁺.

### Step 5: 5-(2-((2-(tert-Butoxycarbonyl)-7-chloro-1,2,3,4-tetrahydroisoquinolin-6-yl)amino)-5-carbamoylpyrimidin-4-yl)thiophene-3-carboxylic acid

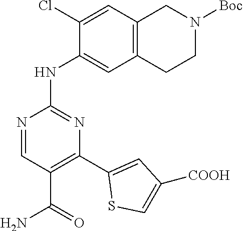 [SMILES: CC(C)(C)OC(=O)N1CCc2cc(Nc3ncc(C(N)=O)c(-c4cc(C(=O)O)cs4)n3)c(Cl)cc2C1]

The title compound was prepared analogously to Example 2, step 1, where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with tert-butyl 6-((5-carbamoyl-4-(4-(methoxycarbonyl)thiophen-2-yl)pyrimidin-2-yl)amino)-7-chloro-3,4-dihydroisoquinoline-2(1H)-carboxylate. The title compound was isolated in 99% yield. m/z (ESI, +ve)=530.2 [M+H]⁺.

### Step 6: tert-Butyl 6-((5-carbamoyl-4-(4-carbamoylthiophen-2-yl)pyrimidin-2-yl)amino)-7-chloro-3,4-dihydroisoquinoline-2(1H)-carboxylate

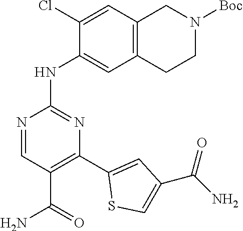 [SMILES: CC(C)(C)OC(=O)N1CCc2cc(Nc3ncc(C(N)=O)c(-c4cc(C(N)=O)cs4)n3)c(Cl)cc2C1]

The title compound was prepared analogously to Example 13 where 5-(2-((7-ethyl-2-methyl-1,2,3,4-tetrahydroisoquinolin-6-yl)amino)-5-(trifluoromethyl)pyrimidin-4-yl)thiophene-3-carboxylic acid hydrochloride was replaced with 5-(2-((2-(tert-Butoxycarbonyl)-7-chloro-1,2,3,4-tetrahydroisoquinolin-6-yl)amino)-5-carbamoylpyrimidin-4-yl)thiophene-3-carboxylic acid. The title compound was isolated in 16% yield. m/z (ESI, +ve)=529.2 [M+H]⁺.

### Step 7

The title compound was prepared analogously to Example 5 where tert-butyl 3-(6-((4-(4-carbamoylthiophen-2-yl)-5-(trifluoromethyl)pyrimidin-2-yl)amino)-7-chloro-3,4-dihydroisoquinolin-2(1H)-yl)azetidine-1-carboxylate was replaced with tert-butyl 6-((5-carbamoyl-4-(4-carbamoylthiophen-2-yl)pyrimidin-2-yl)amino)-7-chloro-3,4-dihydroisoquinoline-2(1H)-carboxylate. The title compound was isolated in 21% yield. m/z (ESI, +ve)=429.3 [M+H]⁺. ¹H NMR (400 MHz, DMSO-d₆) δ 9.13 (s, 1H), 8.41 (s, 1H), 8.32 (d, J=1.3 Hz, 1H), 8.26 (s, 1H), 8.11 (d, J=1.3 Hz, 1H), 8.09 (d, J=2.2 Hz, 1H), 7.88 (s, 1H), 7.72 (d, J=2.2 Hz, 1H), 7.50 (s, 1H), 7.30 (s, 1H), 7.26 (s, 1H), 3.94 (s, 2H), 3.06 (t, J=5.9 Hz, 3H), 2.77 (t, J=6.0 Hz, 2H).

### Example 27: 5-(2-((7-Chloro-1,2,3,4-tetrahydroisoquinolin-6-yl)amino)-5-(trifluoromethyl)pyrimidin-4-yl)thiophene-3-sulfonamide

### Step 1: Methyl 5-bromo-3-sulfamoylthiophene-2-carboxylate

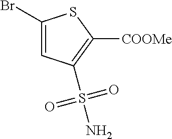 [SMILES: COC(=O)c1sc(Br)cc1S(N)(=O)=O]

Methyl 5-bromo-3-(chlorosulfonyl)thiophene-2-carboxylate (2.48 mmol) was dissolved in a 0.4 N solution of ammonia in 1,4-dioxane (15.5 mL) and the reaction was stirred at room temperature for 20 minutes. Volatiles were removed under reduced pressure and the resulting residue was dissolved in dichloromethane and washed with water. The organic layer was dried with sodium sulfate, filtered, and concentrated to afford the title compound in 96% yield. m/z (ESI, +ve)=300.0 [M+H]⁺.

### Step 2: 5-bromo-3-sulfamoylthiophene-2-carboxylic acid

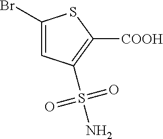 [SMILES: NS(=O)(=O)c1cc(Br)sc1C(=O)O]

The title compound was prepared analogously to Example 8, step 1, where methyl 2-bromothiophene-4-carboxylate was replaced with methyl 5-bromo-3-sulfamoylthiophene-2-carboxylate. The title compound was isolated in 57% yield. m/z (ESI, −ve)=284.0 (M−H)−.

### Step 3: 5-Bromothiophene-3-sulfonamide

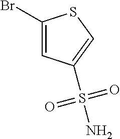

5-Bromo-3-sulfamoylthiophene-2-carboxylic acid (1.05 mmol) was dissolved in DMSO (6.0 mL) and acetic acid (0.006 mL) and silver (I) carbonate (0.052 mmol) were added. The reaction mixture was stirred overnight at 100° C. and quenched with water. Extraction with dichloromethane, filtration and concentration under reduced pressure afforded a crude material that was purified by silica gel chromatography (20-80% ethyl acetate in hexanes). The title compound was isolated in 50% yield. m/z (ESI, +ve)=242.0 [M+H]⁺.

### Step 4: 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-sulfonamide

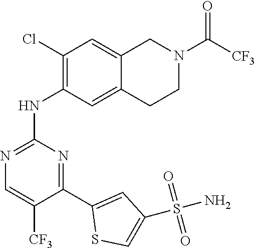 [SMILES: NS(=O)(=O)c1csc(-c2nc(Nc3cc4c(cc3Cl)CN(C(=O)C(F)(F)F)CC4)ncc2C(F)(F)F)c1]

The title compound was prepared analogously to Example 4, step 2, where 2-bromo-6,7-dihydrothieno[3,2-C]pyridin-4(5H)-one was replaced with 5-bromothiophene-3-sulfonamide. The title compound was isolated in 48% yield. m/z (ESI, +ve)=586.1 [M+H]⁺.

### Step 5

The title compound was prepared analogously to Example 4 where 2-(2-((7-chloro-2-(2,2,2-trifluoroacetyl)-1,2,3,4-tetrahydroisoquinolin-6-yl)amino)-5-(trifluoromethyl)pyrimidin-4-yl)-6,7-dihydrothieno[3,2-c]pyridin- 4(5H)-one was replaced with 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-sulfonamide. The title compound was isolated in 40% yield. m/z (ESI, +ve)=490.1 [M+H]⁺. ¹H NMR (400 MHz, DMSO-d₆) δ 9.89 (s, 1H), 8.79 (s, 1H), 8.34 (d, J=1.3 Hz, 1H), 7.89 (s, 1H), 7.57 (s, 2H), 7.29 (s, 1H), 7.21 (s, 1H), 3.84 (s, 2H), 2.93 (s, 2H), 2.68 (s, 2H).

### Example 28: 2-(2-((7-Chloro-1,2,3,4-tetrahydroisoquinolin-6-yl)amino)-5-(trifluoromethyl)pyrimidin-4-yl)-5,6,7,8-tetrahydro-4H-thieno[3,2-c]azepin-4-one

### Step 1: 2-(2-((7-Chloro-2-(2,2,2-trifluoroacetyl)-1,2,3,4-tetrahydroisoquinolin-6-yl)amino)-5-(trifluoromethyl)pyrimidin-4-yl)-5,6,7,8-tetrahydro-4H-thieno[3,2-c]azepin-4-one

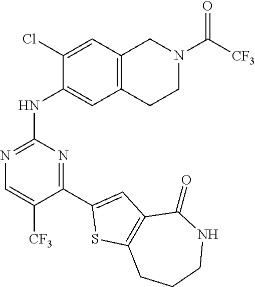 [SMILES: O=C1NCCCc2sc(-c3nc(Nc4cc5c(cc4Cl)CN(C(=O)C(F)(F)F)CC5)ncc3C(F)(F)F)cc21]

The title compound was prepared analogously to Example 4, step 2, where ethyl 2-chlorooxazole-4-carboxylate was replaced with 2-bromo-4H,5H,6H,7H,8H-thieno[3,2-c]azepin-4-one. The title compound was isolated in 24% yield. m/z (ESI, +ve)=590.0 [M+H]⁺.

### Step 2

The title compound was prepared analogously to Example 8 where 1-(7-chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one was replaced with 2-(2-((7-Chloro-2-(2,2,2-trifluoroacetyl)-1,2,3,4-tetrahydroisoquinolin-6-yl)amino)-5-(trifluoromethyl)pyrimidin-4-yl)-5,6,7,8-tetrahydro-4H-thieno[3,2-c]azepin-4-one. The title compound was isolated in 54% yield. m/z (ESI, +ve)=494.2 [M+H]⁺. ¹H NMR (400 MHz, DMSO-d₆) δ 9.74 (s, 1H), 8.73 (s, 1H), 8.03 (t, J=5.3 Hz, 1H), 7.91 (s, 1H), 7.33 (s, 1H), 7.22 (s, 1H), 3.85 (s, 2H), 3.16 (tt, J=5.4, 3.2 Hz, 2H), 3.11 (t, J=6.8 Hz, 2H), 2.95 (t, J=5.8 Hz, 2H), 2.69 (t, J=6.1 Hz, 2H), 1.99 (ddd, J=10.3, 8.5, 5.1 Hz, 2H).

### Example 29: 2-(2-((7-Cyclopropyl-1,2,3,4-tetrahydroisoquinolin-6-yl)amino)-5-(trifluoromethyl)pyrimidin-4-yl)-6,7-dihydrothieno[3,2-c]pyridin-4(5H)-one

### Step 1: 2-(2-((7-Cyclopropyl-2-(2,2,2-trifluoroacetyl)-1,2,3,4-tetrahydroisoquinolin-6-yl)amino)-5-(trifluoromethyl)pyrimidin-4-yl)-6,7-dihydrothieno[3,2-c]pyridin-4(5H)-one

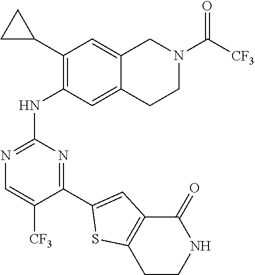 [SMILES: O=C1NCCc2sc(-c3nc(Nc4cc5c(cc4C4CC4)CN(C(=O)C(F)(F)F)CC5)ncc3C(F)(F)F)cc21]

The title compound was prepared analogously to Example 1, step 6, where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one and 4-(methoxycarbonyl)thiophene-2-boronic acid pinacol ester were replaced with 1-(6-((4-chloro-5-(trifluoromethyl)pyrimidin-2-yl)amino)-7-cyclopropyl-3,4-dihydroisoquinolin-2(1H)-yl)-2,2,2-trifluoroethan-1-one and 2-(4,4,5,5-tetramethyl-1,3,2-dioxaborolan-2-yl)-4H,5H,6H,7H-thieno[3,2-c]pyridin-4-one, respectively. The title compound was isolated in 30% yield. m/z (ESI, +ve)=582.2 [M+H]⁺.

### Step 2

The title compound was prepared analogously to Example 1, step 6, where 1-(7-chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one was replaced with 2-(2-((7-cyclopropyl-2-(2,2,2-trifluoroacetyl)-1,2,3,4-tetrahydroisoquinolin-6-yl)amino)-5-(trifluoromethyl)pyrimidin-4-yl)-6,7-dihydrothieno[3,2-c]pyridin-4(5H)-one. The title compound was isolated in 41% yield. m/z (ESI, +ve)=486.4 [M+H]⁺. ¹H NMR (400 MHz, DMSO-d₆) δ 9.59 (s, 1H), 8.71 (s, 1H), 7.83 (s, 1H), 7.78 (s, 1H), 7.14 (s, 1H), 6.65 (s, 1H), 3.79 (s, 2H), 3.47 (td, J=6.9, 2.6 Hz, 2H), 3.05 (t, J=6.8 Hz, 2H), 2.93 (t, J=5.8 Hz, 2H), 2.66 (d, J=5.9 Hz, 2H), 1.93 (m, 1H), 0.85-0.76 (m, 2H), 0.59-0.49 (m, 2H).

### Example 30: 2-(2-((7-Chloro-1,2,3,4-tetrahydroisoquinolin-6-yl)amino)-5-(trifluoromethyl)pyrimidin-4-yl)-5-methylthieno[3,2-c]pyridin-4(5H)-one

### Step 1: 2-(2-((7-Chloro-2-(2,2,2-trifluoroacetyl)-1,2,3,4-tetrahydroisoquinolin-6-yl)amino)-5-(trifluoromethyl)pyrimidin-4-yl)-5-methylthieno[3,2-c]pyridin-4(5H)-one

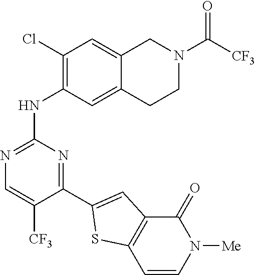 [SMILES: Cn1ccc2sc(-c3nc(Nc4cc5c(cc4Cl)CN(C(=O)C(F)(F)F)CC5)ncc3C(F)(F)F)cc2c1=O]

The title compound was prepared analogously to Example 4, step 2, where 2-bromo-6,7-dihydrothieno[3,2-C]pyridin-4(5H)-one was replaced with 2-bromo-5-methyl-4H,5H-thieno[3,2-c]pyridin-4-one. The title compound was isolated in 45% yield. m/z (ESI, +ve)=588.1 [M+H]⁺.

### Step 2

The title compound was prepared analogously to Example 13 where 1-(7-chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one was replaced with 2-(2-((7-chloro-2-(2,2,2-trifluoroacetyl)-1,2,3,4-tetrahydroisoquinolin-6-yl)amino)-5-(trifluoromethyl)pyrimidin-4-yl)-5-methylthieno[3,2-c]pyridin-4(5H)-one. The title compound was isolated in 56% yield. m/z (ESI, +ve)=492.3 [M+H]⁺. ¹H NMR (400 MHz, DMSO-d₆) δ 9.88 (s, 1H), 8.79 (s, 1H), 8.27 (s, 1H), 8.07 (s, 1H), 7.71 (d, J=7.2 Hz, 1H), 7.36 (s, 1H), 7.28 (s, 1H), 6.95 (d, J=7.2 Hz, 1H), 3.93 (s, 2H), 3.52 (s, 4H, overlapped with water signal), 3.03 (m, 2H), 2.76 m, 2H).

### Example 31: 2-(2-((7-Chloro-1,2,3,4-tetrahydroisoquinolin-6-yl)amino)-5-(trifluoromethyl)pyrimidin-4-yl)-5-methylthieno[2,3-d]pyridazin-4(5H)-one

### Step 1: 2-Bromo-5-methylthieno[2,3-d]pyridazin-4(5H)-one

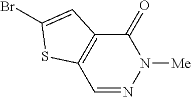 [SMILES: Cn1ncc2sc(Br)cc2c1=O]

A solution of 2-bromothieno[2,3-D]pyrazin-4(5H)-one (0.65 mmol) and dimethylformamide-dimethyl acetal (0.97 mmol) in DMF (3 mL) was stirred at 160° C. for 2 hours in a sealed pressure tube. The reaction mixture was cooled down and concentrated to dryness. The crude was purified by silica gel chromatography (0-50% ethyl acetate in hexanes) to afford the title compound in 72% yield. m/z (ESI, +ve)=246.9 [M+H]⁺.

### Step 2: 2-(2-((7-Chloro-2-(2,2,2-trifluoroacetyl)-1,2,3,4-tetrahydroisoquinolin-6-yl)amino)-5-(trifluoromethyl)pyrimidin-4-yl)-5-methylthieno[2,3-d]pyridazin-4(5H)-one

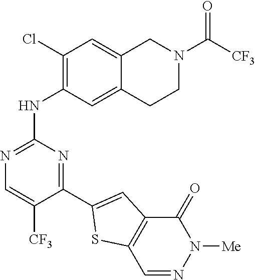 [SMILES: Cn1ncc2sc(-c3nc(Nc4cc5c(cc4Cl)CN(C(=O)C(F)(F)F)CC5)ncc3C(F)(F)F)cc2c1=O]

The title compound was prepared analogously to Example 6, step 2, where 2-bromo-6,7-dihydrothieno[3,2-C]pyridin-4(5H)-one was replaced with 2-bromo-5-methyl-4H,5H-thieno[2,3-d]pyridazin-4-one. The title compound was isolated in 72% yield. m/z (ESI, +ve)=589.1 [M+H]⁺.

### Step 3:

The title compound was prepared analogously to Example 13, where 1-(7-chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one was replaced with 2-(2-((7-chloro-2-(2,2,2-trifluoroacetyl)-1,2,3,4-tetrahydroisoquinolin-6-yl)amino)-5-(trifluoromethyl)pyrimidin-4-yl)-5-methylthieno[2,3-d]pyridazin-4(5H)-one. The title compound was isolated in 38% yield. m/z (ESI, +ve)=493.2 [M+H]⁺. ¹H NMR (400 MHz, DMSO-d₆) δ 10.13 (s, 1H), 8.88 (s, 1H), 8.65 (s, 1H), 8.06 (s, 1H), 7.43 (s, 1H), 7.41 (s, 1H), 4.15 (s, 2H), 3.74 (s, 3H), 3.25 (t, J=6.1 Hz, 4H, overlapped with H2O), 2.90 (t, J=6.2 Hz, 2H).

### Example 32: 7-Chloro-N-(4-(4-(methylsulfonyl)thiophen-2-yl)-5-(trifluoromethyl)pyrimidin-2-yl)-1,2,3,4-tetrahydroisoquinolin-6-amine

### Step 1: 2-(Methoxycarbonyl)thiophene-3-sulfinic acid

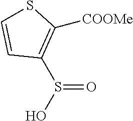 [SMILES: COC(=O)c1sccc1S(=O)O]

A suspension of Na2SO3 (15 mmol) and NaHCO3 (16 mmol) in water (30.0 mL) was heated at 40° C. and methyl 3-chlorosulfonylthiophene-2-carboxylate (12 mmol) was added in small portions. The reaction mixture was stirred at 40° C. for one additional hour, cooled down to room temperature and filtered. The aqueous layer was acidified with a 1M aqueous solution of HCl and extracted with dichloromethane twice. The combined organic layers were dried over sodium sulfate, filtered, and concentrated under reduced pressure to afford the title compound in 70% yield. m/z (ESI, +ve)=207.0 [M+H]⁺.

### Step 2: Methyl 3-(methylsulfonyl)thiophene-2-carboxylate

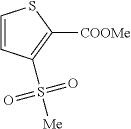 [SMILES: COC(=O)c1sccc1S(C)(=O)=O]

To a solution of 2-(methoxycarbonyl)thiophene-3-sulfinic acid (8.8 mmol) in DMF (18.0 mL), cesium carbonate (17.5 mmol) and methyl iodide (1.1 mL) were added. The reaction mixture was stirred at room temperature for 2 hours, filtered and concentrated under reduced pressure. The residue was purified by silica gel chromatography (30% ethyl acetate in hexanes) to afford the title compound in 52% yield. m/z (ESI, +ve)=221.0 [M+H]⁺.

### Step 3: Methyl 5-bromo-3-(methylsulfonyl)thiophene-2-carboxylate

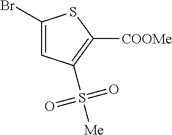 [SMILES: COC(=O)c1sc(Br)cc1S(C)(=O)=O]

Methyl 3-(methylsulfonyl)thiophene-2-carboxylate (1.36 mmol) was dissolved in TFA (6.0 mL) and H₂SO₄ (13.6 mmol). The reaction mixture was cooled down to −15° C. and N-bromosuccinimide (1.49 mmol) was added in small portions over 15 minutes. The cooling bath was removed and the solution was stirred at room temperature for one hour. The reaction was quenched with water, extracted with dichloromethane three times, the combined organic layers were washed with brine, dried over sodium sulfate, filtered, and concentrated to afford a residue that was purified by silica gel chromatography (30% ethyl acetate in hexanes). The title compound was isolated in 42% yield. m/z (ESI, +ve)=300.8 [M+H]⁺.

### Step 4: 5-Bromo-3-(methylsulfonyl)thiophene-2-carboxylic acid

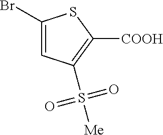 [SMILES: CS(=O)(=O)c1cc(Br)sc1C(=O)O]

A 2.2 M aqueous solution of LiOH (0.5 mL) was added over a solution of methyl 5-bromo-3-methanesulfonylthiophene-2-carboxylate (0.56 mmol) in methanol (3.4 mL) and the reaction was stirred at room temperature for one hour. After evaporation of volatiles under reduced pressure, the crude was dissolved in water and the pH was made acidic by the addition of a 1M aqueous solution of HCl. The precipitate was filtered affording title compound in 93% yield. ¹H NMR (400 MHz, DMSO-d₆) δ 14.9-13.9 (bs, 1H), 7.60 (s, 1H), 3.47 (s, 3H).

### Step 5: 2-Bromo-4-(methylsulfonyl)thiophene

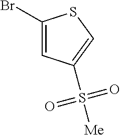

5-Bromo-3-methanesulfonylthiophene-2-carboxylic acid (0.52 mmol) was dissolved in DMSO (1.0 mL). Acetic acid (0.006 mL) and silver carbonate (0.025 mmol) were added and the reaction mixture was stirred at 100° C. for 2 hours. Dilution with water yielded a precipitate that was filtered to afford the title compound in 47% yield.

### Step 6: 1-(7-Chloro-6-((4-(4-(methylsulfonyl)thiophen-2-yl)-5-(trifluoromethyl)pyrimidin-2-yl)amino)-3,4-dihydroisoquinolin-2(1H)-yl)-2,2,2-trifluoroethan-1-one

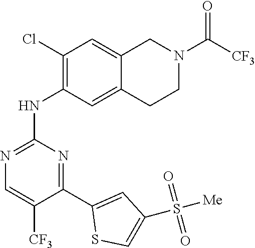 [SMILES: CS(=O)(=O)c1csc(-c2nc(Nc3cc4c(cc3Cl)CN(C(=O)C(F)(F)F)CC4)ncc2C(F)(F)F)c1]

The title compound was prepared analogously to Example 4, step 2, where 2-bromo-6,7-dihydrothieno[3,2-C]pyridin-4(5H)-one was replaced with 2-bromo-4-(methylsulfonyl)thiophene. The title compound was isolated in 44% yield. m/z (ESI, +ve)=585.0 [M+H]⁺

### Step 7

The title compound was prepared analogously to Example 8 where 1-(7-chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one was replaced with 1-(7-chloro-6-((4-(4-(methylsulfonyl)thiophen-2-yl)-5-(trifluoromethyl)pyrimidin-2-yl)amino)-3,4-dihydroisoquinolin-2(1H)-yl)-2,2,2-trifluoroethan-1-one. The title compound was isolated in 24% yield. m/z (ESI, +ve)=489.18 [M+H]⁺. ¹H NMR (400 MHz, DMSO-d₆) δ 10.02 (s, 1H), 8.83 (s, 1H), 8.68 (d, J=1.2 Hz, 1H), 7.90 (s, 1H), 7.37 (s, 1H), 7.33 (s, 1H), 4.02 (s, 2H), 3.30 (s, 3H), 3.13 (t, J=6.1 Hz, 2H), 2.81 (t, J=6.0 Hz, 2H), 2.08 (s, 1H).

### Example 33: 2-(2-((6-Chloroisoindolin-5-yl)amino)-5-(trifluoromethyl)pyrimidin-4-yl)-6,7-dihydrothieno[3,2-c]pyridin-4(5H)-one

### Step 1: 2-(2-((6-Chloro-2-(2,2,2-trifluoroacetyl)isoindolin-5-yl)amino)-5-(trifluoromethyl)pyrimidin-4-yl)-6,7-dihydrothieno[3,2-c]pyridin-4(5H)-one

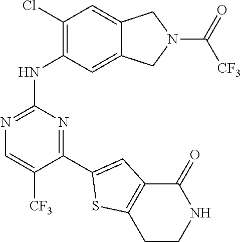 [SMILES: O=C1NCCc2sc(-c3nc(Nc4cc5c(cc4Cl)CN(C(=O)C(F)(F)F)C5)ncc3C(F)(F)F)cc21]

The title compound was prepared analogously to Example 14, step 2, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one was replaced with 1-(5-chloro-6-((4-chloro-5-(trifluoromethyl)pyrimidin-2-yl)amino)isoindolin-2-yl)-2,2,2-trifluoroethan-1-one. The title compound was isolated in 90% yield. m/z (ESI, +ve)=562.0 [M+H]⁺.

### Step 2

The title compound was prepared analogously to Example 8 where 1-(7-chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one was replaced with 2-(2-((6-chloro-2-(2,2,2-trifluoroacetyl)isoindolin-5-yl)amino)-5-(trifluoromethyl)pyrimidin-4-yl)-6,7-dihydrothieno[3,2-c]pyridin-4(5H)-one. The title compound was isolated in 26% yield.

m/z (ESI, +ve)=466.1 [M+H]⁺. ¹H NMR (400 MHz, DMSO-d₆) δ 9.87 (s, 1H), 8.75 (s, 1H), 8.18 (s, 1H), 7.83 (s, 1H), 7.78 (s, 1H), 7.49 (s, 1H), 7.48 (s, 1H), 4.16 (s, 4H), 3.46 (td, J=6.8, 2.6 Hz, 2H), 3.04 (t, J=6.7 Hz, 2H).

### Example 34: 2-(2-((7-ethyl-1,2,3,4-tetrahydroisoquinolin-6-yl)amino)-5-(trifluoromethyl)pyrimidin-4-yl)-6,7-dihydrothieno[3,2-c]pyridin-4(5H)-one

### Step 1: 2-(2-((7-Ethyl-2-(2,2,2-trifluoroacetyl)-1,2,3,4-tetrahydroisoquinolin-6-yl)amino)-5-(trifluoromethyl)pyrimidin-4-yl)-6,7-dihydrothieno[3,2-c]pyridin-4(5H)-one

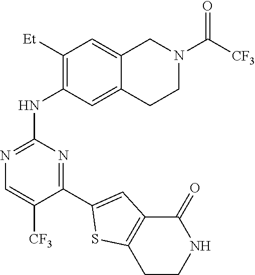 [SMILES: CCc1cc2c(cc1Nc1ncc(C(F)(F)F)c(-c3cc4c(s3)CCNC4=O)n1)CCN(C(=O)C(F)(F)F)C2]

The title compound was prepared analogously to Example 14, step 2, where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one. The title compound was isolated in 24% yield. m/z (ESI, +ve)=570.2 [M+H]⁺.

### Step 2

The title compound was prepared analogously to Example 8 where 1-(7-chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one was replaced with 2-(2-((7-ethyl-2-(2,2,2-trifluoroacetyl)-1,2,3,4-tetrahydroisoquinolin-6-yl)amino)-5-(trifluoromethyl)pyrimidin-4-yl)-6,7-dihydrothieno[3,2-c]pyridin-4(5H)-one. The title compound was isolated in 40% yield. m/z (ESI, +ve)=474.2 [M+H]⁺. ¹H NMR (400 MHz, DMSO-d₆) δ 9.65 (s, 1H), 8.70 (s, 1H), 8.35 (s, 1H), 7.82 (s, 1H), 7.79 (s, 1H), 7.12 (s, 1H), 6.98 (s, 1H), 3.95 (s, 2H), 3.50-3.44 (m, 2H, overlapped with water signal), 3.08-3.00 (m, 4H), 2.75 (s, 2H), 2.62-2.54 (m, 2H), 1.08 (t, J=7.5 Hz, 3H).

### Example 35: 2-(2-((7-(Methylthio)-1,2,3,4-tetrahydroisoquinolin-6-yl)amino)-5-(trifluoromethyl)pyrimidin-4-yl)-6,7-dihydrothieno[3,2-c]pyridin-4(5H)-one

### Step 1: 2,2,2-Trifluoro-1-(7-(methylthio)-6-nitro-3,4-dihydroisoquinolin-2(1H)-yl)ethan-1-one

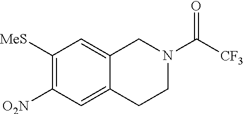 [SMILES: CSc1cc2c(cc1[N+](=O)[O-])CCN(C(=O)C(F)(F)F)C2]

1-(7-Chloro-6-nitro-1,2,3,4-tetrahydroisoquinolin-2-yl)-2,2,2-trifluoroethan-1-one (3.2 mmol) was dissolved in DMSO (20 mL) and. NaSMe (3.6 mmol) was added in one portion. After 16 hours at room temperature, the reaction was diluted with water, extracted with dichloromethane, dried over anhydrous sodium sulfate, filtered and concentrated. Purification in silica gel (15-30% ethyl acetate in hexanes) afforded the title compound in 28% yield. m/z (ESI, +ve)=319.1 [M+H]⁺

### Step 2: 1-(6-Amino-7-(methylthio)-3,4-dihydroisoquinolin-2(1H)-yl)-2,2,2-trifluoroethan-1-one

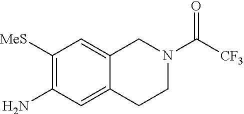 [SMILES: CSc1cc2c(cc1N)CCN(C(=O)C(F)(F)F)C2]

The title compound was prepared analogously to Example 9, step 3, where 1-(7-ethyl-6-nitro-1,2,3,4-tetrahydroisoquinolin-2-yl)-2,2,2-trifluoroethan-1-one was replaced with 2,2,2-Trifluoro-1-(7-(methylthio)-6-nitro-3,4-dihydroisoquinolin-2(1H)-yl)ethan-1-one. The title compound was isolated in 84% yield. m/z (ESI, +ve)=291.1 [M+H]⁺

### Step 3: 1-(6-((4-Chloro-5-(trifluoromethyl)pyrimidin-2-yl)amino)-7-(methylthio)-3,4-dihydroisoquinolin-2(1H)-yl)-2,2,2-trifluoroethan-1-one

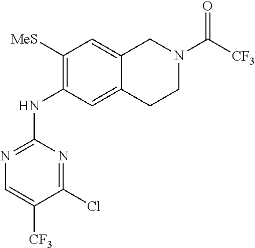 [SMILES: CSc1cc2c(cc1Nc1ncc(C(F)(F)F)c(Cl)n1)CCN(C(=O)C(F)(F)F)C2]

The title compound was prepared analogously to Example 9, step 4, where 1-(6-amino-7-ethyl-3,4-dihydroisoquinolin-2(1H)-yl)-2,2,2-trifluoroethan-1-one was replaced with 1-(6-amino-7-(methylthio)-3,4-dihydroisoquinolin-2(1H)-yl)-2,2,2-trifluoroethan-1-one. The title compound was isolated in 22% yield. m/z (ESI, +ve)=471.1 [M+H]⁺

### Step 4: 2-(2-((7-(Methylthio)-2-(2,2,2-trifluoroacetyl)-1,2,3,4-tetrahydroisoquinolin-6-yl)amino)-5-(trifluoromethyl)pyrimidin-4-yl)-6,7-dihydrothieno[3,2-c]pyridin-4(5H)-one

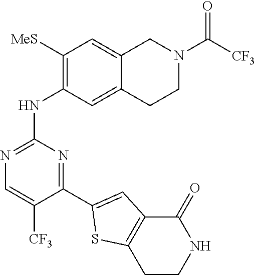 [SMILES: CSc1cc2c(cc1Nc1ncc(C(F)(F)F)c(-c3cc4c(s3)CCNC4=O)n1)CCN(C(=O)C(F)(F)F)C2]

The title compound was prepared analogously to Example 1, step 6 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one and 4-(methoxycarbonyl)thiophene-2-boronic acid pinacol ester were replaced with 1-(6-((4-Chloro-5-(trifluoromethyl)pyrimidin-2-yl)amino)-7-(methylthio)-3,4-dihydroisoquinolin-2(1H)-yl)-2,2,2-trifluoroethan-1-one and 2-(4,4,5,5-tetramethyl-1,3,2-dioxaborolan-2-yl)-4H,5H,6H,7H-thieno[3,2-c]pyridin-4-one. The title compound was isolated in 46% yield. m/z (ESI, +ve)=588.1 [M+H]⁺.

### Step 5

The title compound was prepared analogously to Example 8 where 1-(7-chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one was replaced with 2-(2-((7-(Methylthio)-2-(2,2,2-trifluoroacetyl)-1,2,3,4-tetrahydroisoquinolin-6-yl)amino)-5-(trifluoromethyl)pyrimidin-4-yl)-6,7-dihydrothieno[3,2-c]pyridin-4(5H)-one. The title compound was isolated in 50% yield. m/z (ESI, +ve)=492.1 [M+H]⁺. ¹H NMR (400 MHz, DMSO-d₆) δ 9.59 (s, 1H), 8.71 (s, 1H), 8.24 (s, 1H), 7.82 (s, 1H), 7.78 (s, 1H), 7.20 (s, 1H), 7.11 (s, 1H), 3.99 (s, 2H), 3.47 (td, J=6.8, 2.6 Hz, 2H), 3.06 (q, J=7.5, 6.9 Hz, 4H), 2.75 (t, J=5.8 Hz, 2H), 2.36 (s, 3H).

### Example 36: 2-(2-((2-Cyclopropyl-4-(4-methylpiperazin-1-yl)phenyl)amino)-5-(trifluoromethyl)pyrimidin-4-yl)-6,7-dihydrothieno[3,2-c]pyridin-4(5H)-one

### Step 1: 2-Cyclopropyl-4-(4-methylpiperazin-1-yl)aniline

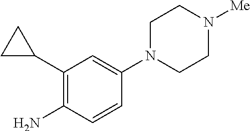 [SMILES: CN1CCN(c2ccc(N)c(C3CC3)c2)CC1]

A solution of 1-(3-cyclopropyl-4-nitrophenyl)-4-methylpiperazine (7.65 mmol) in methanol (10 mL) at 0° C., was treated with NH₄Cl (2.33 mmol) and the reaction mixture was stirred at 0° C. for 10 minutes. Zinc dust (76.5 mmol) was added slowly and after 15 minutes the reaction mixture was allowed to warm to room temperature and stirred overnight. Filtration through celite and evaporation of volatiles afforded a residue that was purified by silica gel chromatography (0-10% methanol in dichloromethane) to afford the title compound in 53% yield. m/z (ESI, +ve)=232.2 [M+H]⁺

### Step 2: 4-Chloro-N-(2-cyclopropyl-4-(4-methylpiperazin-1-yl)phenyl)-5-(trifluoromethyl)pyrimidin-2-amine

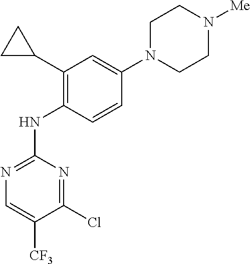 [SMILES: CN1CCN(c2ccc(Nc3ncc(C(F)(F)F)c(Cl)n3)c(C3CC3)c2)CC1]

A 0.7 M solution of ZnCl2 in THF (6.8 mL) was added dropwise over a solution of 2,4-dichloro-5-trifluoromethyl-pyrimidine (2.17 mmol) in dichloroethane (9 mL) and tert-butanol (1.5 mL). The reaction mixture was stirred at 0° C. for 1 hour and a solution of 2-cyclopropyl-4-(4-methylpiperazin-1-yl)aniline (2.16 mmol) in dichloroethane (2.5 mL) and tert-butanol (2.5 mL) was added, followed by triethylamine (0.42 mL). After 16 hours at room temperature, the reaction was concentrated under reduced pressure and the crude was purified by silica gel chromatography (0-10% methanol in dichloromethane) to afford the title compound in 12% yield. m/z (ESI, +ve)=412.3 [M+H]⁺.

### Step 3

The title compound was prepared analogously to Example 1, step 6, where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one and 4-(methoxycarbonyl)thiophene-2-boronic acid pinacol ester were replaced with 4-chloro-N-(2-cyclopropyl-4-(4-methylpiperazin-1-yl)phenyl)-5-(trifluoromethyl)pyrimidin-2-amine and dihydrothienopyridin-4-one-2-boronic acid pinacol ester, respectively. The title compound was isolated in 11% yield. m/z (ESI, +ve)=529.2 [M+H]⁺. ¹H NMR (400 MHz, DMSO-d₆) 6.53 (s, 1H), 8.67 (s, 1H), 7.80 (s, 1H), 7.78 (s, 1H), 7.20 (d, J=9.8 Hz, 1H), 6.77 (d, J=9.3 Hz, 1H), 6.48 (s, 1H), 3.46 (s, 2H), 3.11 (m, 4H), 3.04 (s, 2H), 2.45 (m, 5H), 2.22 (s, 3H), 1.93 (s, 1H), 0.84-0.76 (m, 2H), 0.65-0.57 (m, 2H).

### Example 37: 5-(2-((2-Ethyl-4-(4-methylpiperazin-1-yl)phenyl)amino)-5-(trifluoromethyl)pyrimidin-4-yl)thiophene-3-carboxamide

### Step 1: 1-Methyl-4-(4-nitro-3-vinylphenyl)piperazine

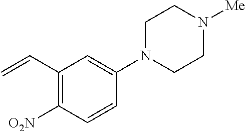 [SMILES: C=Cc1cc(N2CCN(C)CC2)ccc1[N+](=O)[O-]]

A mixture of 1-(3-bromo-4-nitrophenyl)-4-methylpiperazine (16.7 mmol), potassium vinyltrifluoroborate (25.0 mmol), potassium carbonate (49.9 mmol) and Pd(dppf)Cl2-dichloromethane complex (0.83 mmol) in DMSO (35 mL), was stirred at 80° C. for 3 hours. The reaction was cooled down to room temperature, diluted with water and extracted with dichloromethane three times. The combined organic layers were concentrated and the resulting crude purified by silica gel chromatography (0-8% MeOH in dichloromethane) to afford the title compound in 92% yield.

### Step 2: 2-Ethyl-4-(4-methylpiperazin-1-yl)aniline

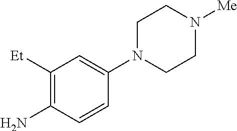 [SMILES: CCc1cc(N2CCN(C)CC2)ccc1N]

The title compound was prepared analogously to Example 19, step 3 where 1-(5-cyclopropyl-6-nitroisoindolin-2-yl)-2,2,2-trifluoroethan-1-one was replaced with 1-methyl-4-(4-nitro-3-vinylphenyl)piperazine. The title compound was isolated in 89% yield m/z (ESI, +ve)=220.5 [M+H]⁺

### Step 3: 4-Chloro-N-(2-ethyl-4-(4-methylpiperazin-1-yl)phenyl)-5-(trifluoromethyl)pyrimidin-2-amine

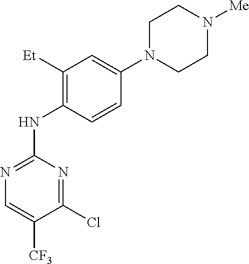 [SMILES: CCc1cc(N2CCN(C)CC2)ccc1Nc1ncc(C(F)(F)F)c(Cl)n1]

The title compound was prepared analogously to Example 36, step 2, where 2-cyclopropyl-4-(4-methylpiperazin-1-yl)aniline was replaced with 2-ethyl-4-(4-methylpiperazin-1-yl)aniline. The title compound was isolated in 53% yield. m/z (ESI, +ve)=400.5 [M+H]⁺.

### Step 4: Methyl 5-(2-((2-ethyl-4-(4-methylpiperazin-1-yl)phenyl)amino)-5-(trifluoromethyl)pyrimidin-4-yl)thiophene-3-carboxylate

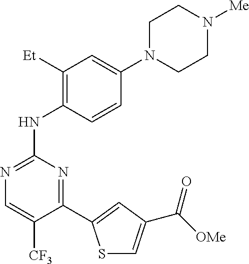 [SMILES: CCc1cc(N2CCN(C)CC2)ccc1Nc1ncc(C(F)(F)F)c(-c2cc(C(=O)OC)cs2)n1]

The title compound was prepared analogously to Example 1, step 6 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was substituted with 4-chloro-N-(2-ethyl-4-(4-methylpiperazin-1-yl)phenyl)-5-(trifluoromethyl)pyrimidin-2-amine. The title compound was isolated in 28% yield. m/z (ESI, +ve)=506.6 [M+H]⁺.

### Step 5: 5-(2-((2-ethyl-4-(4-methylpiperazin-1-yl)phenyl)amino)-5-(trifluoromethyl)pyrimidin-4-yl)thiophene-3-carboxylic acid

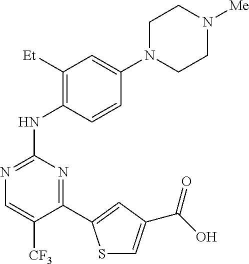 [SMILES: CCc1cc(N2CCN(C)CC2)ccc1Nc1ncc(C(F)(F)F)c(-c2cc(C(=O)O)cs2)n1]

The title compound was prepared analogously to Example 2, step 1 where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was substituted with methyl 5-(2-((2-ethyl-4-(4-methylpiperazin-1-yl)phenyl)amino)-5-(trifluoromethyl)pyrimidin-4-yl)thiophene-3-carboxylate. The title compound was isolated in 28% yield. m/z (ESI, +ve)=492.3 [M+H]⁺.

### Step 6

The title compound was prepared analogously to Example 13, where 5-(2-((7-ethyl-2-methyl-1,2,3,4-tetrahydroisoquinolin-6-yl)amino)-5-(trifluoromethyl)pyrimidin-4-yl)thiophene-3-carboxylic acid hydrochloride was replaced with 5-(2-((2-ethyl-4-(4-methylpiperazin-1-yl)phenyl)amino)-5-(trifluoromethyl)pyrimidin-4-yl)thiophene-3-carboxylic acid. The title compound was isolated in 16% yield. m/z (ESI, +ve)=491.2 [M+H]⁺. ¹H NMR (300 MHz, MeOH-d4) δ. 8.56 (s, 1H), 8.26 (s, 1H), 8.12 (s, 1H), 7.25 (s, 1H), 6.97-6.81 (m, 2H), 3.23 (d, J=5.2 Hz, 4H), 2.74-2.54 (m, 6H), 2.39 (s, 3H), 1.17 (t, J=7.6 Hz, 3H).

### Example 38: (5-(2-((7-Chloro-1,2,3,4-tetrahydroisoquinolin-6-yl)amino)-5-(trifluoromethyl)pyrimidin-4-yl)thiophen-3-yl)dimethylphosphine oxide

### Step 1: Dimethyl(thiophen-3-yl)phosphine oxide

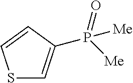

A solution of 3-iodothiophene (4.76 mmol), dimethylphosphine oxide (4.32 mmol), triethylamine (0.75 mL), Pd₂(dba)₃ (0.044 mmol) and Xantphos (0.044 mmol) in 1,4-dioxane (22 mL), was stirred at room temperature for 20 hours. The reaction was concentrated and purified by silica gel chromatography (3-5% methanol in dichloromethane) to afford the title compound in 55% yield.

### Step 2: (5-Iodothiophen-3-yl)dimethylphosphine oxide

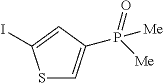

A solution of 3-(dimethylphosphoryl)thiophene in ethanol (6.6 mL) was treated with I₂ (0.94 mmol) and H₅IO₆ (1.09 mmol) at room temperature. The resulting mixture was stirred at 80° C. for 30 minutes and at room temperature overnight. The reaction was concentrated under reduced pressure and the residue purified by silica gel chromatography (5% methanol in dichloromethane) to afford the title compound in 55% yield.

### Step 3: 1-(7-Chloro-6-((4-(4-(dimethylphosphoryl)thiophen-2-yl)-5-(trifluoromethyl)pyrimidin-2-yl)amino)-3,4-dihydroisoquinolin-2(1H)-yl)-2,2,2-trifluoroethan-1-one

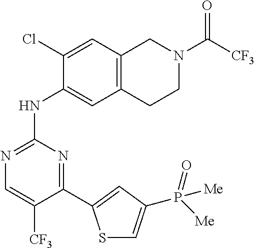 [SMILES: CP(C)(=O)c1csc(-c2nc(Nc3cc4c(cc3Cl)CN(C(=O)C(F)(F)F)CC4)ncc2C(F)(F)F)c1]

The title compound was prepared analogously to Example 4, step 2, where 2-bromo-6,7-dihydrothieno[3,2-C]pyridin-4(5H)-one was replaced with 4-(dimethylphosphoryl)-2-iodothiophene. The title compound was isolated in 50% yield. m/z (ESI, +ve)=583.0 [M+H]⁺.

### Step 4

The title compound was prepared analogously to Example 8 where 1-(7-chloro-6-((4-(4-(4,5-dihydrooxazol-2-yl)thiophen-2-yl)-5-(trifluoromethyl)pyrimidin-2-yl)amino)-3,4-dihydroisoquinolin-2(1H)-yl)-2,2,2-trifluoroethan-1-one was replaced with 1-(7-chloro-6-((4-(4-(dimethylphosphoryl)thiophen-2-yl)-5-(trifluoromethyl)pyrimidin-2-yl)amino)-3,4-dihydroisoquinolin-2(1H)-yl)-2,2,2-trifluoroethan-1-one. The title compound was isolated in 52% yield. m/z (ESI, +ve)=487.1 [M+H]⁺. ¹H NMR (400 MHz, DMSO-d₆) δ 9.84 (s, 1H), 8.78 (s, 1H), 8.35 (dd, J=7.1, 1.2 Hz, 1H), 7.85 (d, J=4.1 Hz, 1H), 7.31 (s, 1H), 7.22 (s, 1H), 3.85 (s, 2H), 3.01-2.89 (m, 2H), 2.72-2.64 (m, 2H), 1.67 (d, J=13.6 Hz, 6H).

### Example 39: 2-(2-((2-ethyl-4-(4-methylpiperazin-1-yl)phenyl)amino)-5-(trifluoromethyl)pyrimidin-4-yl)-6,7-dihydrothieno[3,2-c]pyridin-4(5H)-one

The title compound was prepared analogously to Example 1, step 6, where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one and 4-(methoxycarbonyl)thiophene-2-boronic acid pinacol ester were replaced with 4-chloro-N-[2-ethyl-4-(4-methylpiperazin-1-yl)phenyl]-5-(trifluoromethyl)pyrimidin-2-amine and 2-(4,4,5,5-tetramethyl-1,3,2-dioxaborolan-2-yl)-4H,5H,6H,7H-thieno[3,2-c]pyridin-4-one, respectively. The title compound was isolated in 10% yield. m/z (ESI, +ve)=517.1 [M+H]⁺. ¹H NMR (300 MHz, MeOH-d4) δ 8.54 (s, 1H), 8.01 (s, 1H), 7.29 (d, J=8.3 Hz, 1H), 6.99-6.84 (m, 2H), 3.60 (t, J=6.9 Hz, 2H), 3.36 (s, 5H), 3.10 (d, J=5.3 Hz, 5H), 2.71 (s, 3H), 2.63 (q, J=7.6 Hz, 2H), 1.17 (t, J=7.6 Hz, 3H).

### Example 43: 2-(2-((7-fluoro-1,2,3,4-tetrahydroisoquinolin-6-yl)amino)-5-(trifluoromethyl)pyrimidin-4-yl)-6,7-dihydrothieno[3,2-c]pyridin-4(5H)-one

### Step 1: 1-(6-amino-7-fluoro-3,4-dihydroisoquinolin-2(1H)-yl)-2,2,2-trifluoroethan-1-one

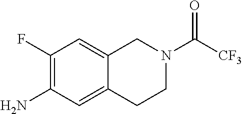 [SMILES: Nc1cc2c(cc1F)CN(C(=O)C(F)(F)F)CC2]

The title compound was prepared analogously to Example 1, steps 1-4, where 2-(4-chlorophenyl)ethylamine was replaced with 2-(4-fluorophenyl)ethan-1-amine in step 1. MS (ESI) m/z: 263.0 [M+H]⁺.

### Step 2: 1-(6-((4-chloro-5-(trifluoromethyl)pyrimidin-2-yl)amino)-7-fluoro-3,4-dihydroisoquinolin-2(1H)-yl)-2,2,2-trifluoroethan-1-one

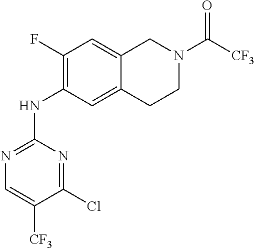 [SMILES: O=C(N1CCc2cc(Nc3ncc(C(F)(F)F)c(Cl)n3)c(F)cc2C1)C(F)(F)F]

The title compound was prepared analogously to Example 1, step 5, where 1-(6-amino-7-chloro-1,2,3,4-tetrahydroisoquinolin-2-yl)-2,2,2-trifluoroethan-1-one was replaced with 1-(6-amino-7-fluoro-3,4-dihydroisoquinolin-2(1H)-yl)-2,2,2-trifluoroethan-1-one. The title compound was isolated in 30% yield. MS (ESI) m/z: 443.0 [M+H]⁺.

### Step 3: 2-(2-((7-fluoro-2-(2,2,2-trifluoroacetyl)-1,2,3,4-tetrahydroisoquinolin-6-yl)amino)-5-(trifluoromethyl)pyrimidin-4-yl)-6,7-dihydrothieno[3,2-c]pyridin-4(5H)-one

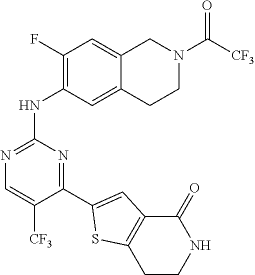 [SMILES: O=C1NCCc2sc(-c3nc(Nc4cc5c(cc4F)CN(C(=O)C(F)(F)F)CC5)ncc3C(F)(F)F)cc21]

The title compound was prepared analogously to Example 1, step 6 where 4-(methoxycarbonyl)thiophene-2-boronic acid pinacol ester was replaced with 2-(4,4,5,5-tetramethyl-1,3,2-dioxaborolan-2-yl)-4H,5H,6H,7H-thieno[3,2-c]pyridin-4-one. The title compound was isolated in 37% yield. MS (ESI) m/z: 560.2 [M+H]⁺.

### Step 4: 2-(2-((7-fluoro-1,2,3,4-tetrahydroisoquinolin-6-yl)amino)-5-(trifluoromethyl)pyrimidin-4-yl)-6,7-dihydrothieno[3,2-c]pyridin-4(5H)-one

The title compound was prepared analogously to Example 1, step 7 where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 2-(2-((7-fluoro-2-(2,2,2-trifluoroacetyl)-1,2,3,4-tetrahydroisoquinolin-6-yl)amino)-5-(trifluoromethyl)pyrimidin-4-yl)-6,7-dihydrothieno[3,2-c]pyridin-4(5H)-one. The title compound was isolated in 15% yield. MS (ESI) m/z: 463.8 [M+H]⁺. 1H-NMR (400 MHz, DMSO-d₆) δ 9.90 (s, 1H), 8.76 (s, 1H), 8.42 (s, 2H), 7.84 (s, 1H), 7.79 (s, 1H), 7.30 (s, 1H), 6.98 (d, J=11.0 Hz, 1H), 3.06 (t, J=6.7 Hz, 2H).

### Example 46: 2-(2-((6-chloro-1,2,3,4-tetrahydroisoquinolin-7-yl)amino)-5-(trifluoromethyl)pyrimidin-4-yl)-5,6,7,8-tetrahydro-4H-thieno[3,2-c]azepin-4-one

### Step 1: 1-(6-chloro-3,4-dihydroisoquinolin-2(1H)-yl)-2,2,2-trifluoroethan-1-one

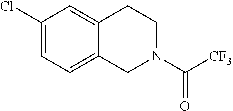 [SMILES: O=C(N1CCc2cc(Cl)ccc2C1)C(F)(F)F]

Trifluoroacetic anhydride (72 mmol) was added over a 0° C. solution of 6-chloro-1,2,3,4-tetrahydroisoquinoline (60 mmol) and triethylamine (119 mmol) in dichloromethane (300 mL) and stirred for one hour. The reaction mixture was diluted with water, extracted with ethyl acetate three times and the combined organic layers were dried over anhydrous sodium sulfate, filtered, and concentrated under reduced pressure to afford the title compound in 96% yield. MS (ESI) m/z: 263.9 [M+H]⁺.

### Step 2: 1-(6-chloro-7-nitro-3,4-dihydroisoquinolin-2(1H)-yl)-2,2,2-trifluoroethan-1-one

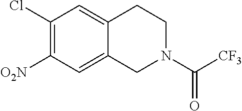 [SMILES: O=C(N1CCc2cc(Cl)c([N+](=O)[O-])cc2C1)C(F)(F)F]

The title compound was prepared analogously to Example 1, step 3 where 1-(7-chloro-1,2,3,4-tetrahydroisoquinolin-2-yl)-2,2,2-trifluoroethan-1-one was replaced with 1-(6-chloro-3,4-dihydroisoquinolin-2(1H)-yl)-2,2,2-trifluoroethan-1-one. The title compound was isolated in 79% yield. MS (ESI) m/z: 307.1 [M+H]⁺.

### Step 3: 1-(7-amino-6-chloro-3,4-dihydroisoquinolin-2(1H)-yl)-2,2,2-trifluoroethan-1-one

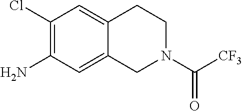 [SMILES: Nc1cc2c(cc1Cl)CCN(C(=O)C(F)(F)F)C2]

The title compound was prepared analogously to Example 1, step 4 where 1-(7-chloro-6-nitro-1,2,3,4-tetrahydroisoquinolin-2-yl)-2,2,2-trifluoroethan-1-one was replaced with 1-(6-chloro-7-nitro-3,4-dihydroisoquinolin-2(1H)-yl)-2,2,2-trifluoroethan-1-one. The title compound was isolated in 96% yield. MS (ESI) m/z: 279.1 [M+H]⁺.

### Step 4: 1-(6-chloro-7-((4-chloro-5-(trifluoromethyl)pyrimidin-2-yl)amino)-3,4-dihydroisoquinolin-2(1H)-yl)-2,2,2-trifluoroethan-1-one

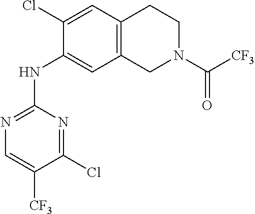 [SMILES: O=C(N1CCc2cc(Cl)c(Nc3ncc(C(F)(F)F)c(Cl)n3)cc2C1)C(F)(F)F]

The title compound was prepared analogously to Example 1, step 5 where 1-(6-amino-7-chloro-1,2,3,4-tetrahydroisoquinolin-2-yl)-2,2,2-trifluoroethan-1-one was replaced with 1-(7-amino-6-chloro-3,4-dihydroisoquinolin-2(1H)-yl)-2,2,2-trifluoroethan-1-one. The title compound was isolated in 49% yield. MS (ESI) m/z: 459.0 [M+H]⁺.

### Step 5: 2-(4,4,5,5-tetramethyl-1,3,2-dioxaborolan-2-yl)-5,6,7,8-tetrahydro-4H-thieno[3,2-c]azepin-4-one

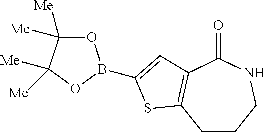 [SMILES: CC1(C)OB(c2cc3c(s2)CCCNC3=O)OC1(C)C]

A solution of 2-bromo-4H,5H,6H,7H,8H-thieno[3,2-c]azepin-4-one (0.41 mmol), potassium acetate (0.61 mmol), bis(pinacolato)diboron (0.45 mmol), tricyclohexylphosphine (0.03 mmol) and tris(dibenzylideneacetone)dipalladium(0) (0.012 mmol) in 1,4-dioxane (2 mL) was microwaved at 120° C. for 25 minutes. The solids were filtered off and the volatiles were removed under reduced pressure to afford a residue that was redissolved in dichloromethane (3 mL). Addition of hexanes (30 mL) and storage of this mixture at −20° C. afford a grey solid that was filtered and dried. The title compound was isolated in 63% yield. MS (ESI) m/z: 294.1 [M+H]⁺.

### Step 6: 2-(2-((6-chloro-2-(2,2,2-trifluoroacetyl)-1,2,3,4-tetrahydroisoquinolin-7-yl)amino)-5-(trifluoromethyl)pyrimidin-4-yl)-5,6,7,8-tetrahydro-4H-thieno[3,2-c]azepin-4-one

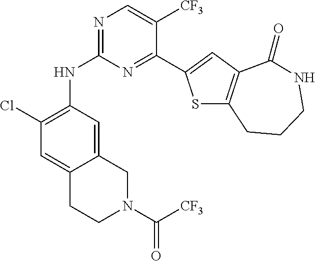 [SMILES: O=C1NCCCc2sc(-c3nc(Nc4cc5c(cc4Cl)CCN(C(=O)C(F)(F)F)C5)ncc3C(F)(F)F)cc21]

The title compound was prepared analogously to Example 1, step 6 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one and 4-(methoxycarbonyl)thiophene-2-boronic acid pinacol ester were replaced with 1-(6-chloro-7-((4-chloro-5-(trifluoromethyl)pyrimidin-2-yl)amino)-3,4-dihydroisoquinolin-2(1H)-yl)-2,2,2-trifluoroethan-1-one and 2-(4,4,5,5-tetramethyl-1,3,2-dioxaborolan-2-yl)-5,6,7,8-tetrahydro-4H-thieno[3,2-c]azepin-4-one. The title compound was isolated in 29% yield. MS (ESI) m/z: 590.0 [M+H]⁺.

### Step 7: 2-(2-((6-chloro-1,2,3,4-tetrahydroisoquinolin-7-yl)amino)-5-(trifluoromethyl)pyrimidin-4-yl)-5,6,7,8-tetrahydro-4H-thieno[3,2-c]azepin-4-one

The title compound was prepared analogously to Example 1, step 7 where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 2-(2-((6-chloro-2-(2,2,2-trifluoroacetyl)-1,2,3,4-tetrahydroisoquinolin-7-yl)amino)-5-(trifluoromethyl)pyrimidin-4-yl)-5,6,7,8-tetrahydro-4H-thieno[3,2-c]azepin-4-one. The title compound was isolated in 31% yield. MS (ESI) m/z: 494.08 [M+H]⁺. (400 MHz, DMSO-d₆) δ 1.95-2.07 (m, 2H), 2.73 (t, J=5.9 Hz, 2H), 3.00 (t, J=6.0 Hz, 2H), 3.11 (t, J=6.8 Hz, 2H), 3.13-3.19 (m, 2H), 3.89 (s, 2H), 7.28 (s, 1H), 7.31 (s, 1H), 7.91 (s, 1H), 8.03 (t, J=5.3 Hz, 1H), 8.26 (s, 1H), 8.73 (s, 1H), 9.76 (s, 1H).

### Example 48: 2-(2-((3-cyclopropyl-1-(1-methylpiperidin-4-yl)-1H-pyrazol-4-yl)amino)-5-(trifluoromethyl)pyrimidin-4-yl)-6,7-dihydrothieno[3,2-c]pyridin-4(5H)-one

### Step 1: tert-butyl 4-(3-cyclopropyl-4-nitro-1H-pyrazol-1-yl)piperidine-1-carboxylate

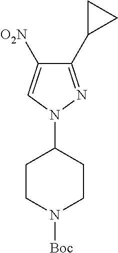 [SMILES: CC(C)(C)OC(=O)N1CCC(n2cc([N+](=O)[O-])c(C3CC3)n2)CC1]

3-Cyclopropyl-4-nitro-1H-pyrazole (13 mmol), tert-butyl 4-hydroxypiperidine-1-carboxylate (14 mmol) and triphenylphosphine (40 mmol) were dissolved in THF (22.0 mL) and the mixture was cooled down to 0° C. A 40% solution of DEAD in toluene (40 mmol) was added dropwise over 25 minutes and the reaction mixture was stirred at room temperature overnight. The volatiles were removed under reduced pressure and the resulting residue was purified by silica gel chromatography (0-30% ethyl acetate in hexanes) to afford the title compound in 51% yield. (400 MHz, DMSO-d₆) δ 8.82 (s, 1H), 4.32 (tt, J=11.6, 4.0 Hz, 1H), 4.02 (d, J=13.3 Hz, 2H), 2.86 (s, 2H), 2.05-1.91 (m, 2H), 1.75 (qd, J=12.2, 4.4 Hz, 2H), 1.41 (s, 9H), 1.31-1.10 (m, 1H), 1.05-0.96 (m, 2H), 0.87 (dq, J=5.0, 3.7 Hz, 2H).

### Step 2: tert-butyl 4-(4-amino-3-cyclopropyl-1H-pyrazol-1-yl)piperidine-1-carboxylate

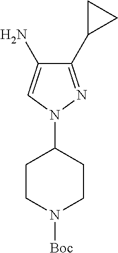 [SMILES: CC(C)(C)OC(=O)N1CCC(n2cc(N)c(C3CC3)n2)CC1]

A suspension of tert-butyl 4-(3-cyclopropyl-4-nitro-1H-pyrazol-1-yl)piperidine-1-carboxylate (6.3 mmol) and 10% palladium on carbon (0.075 mmol) in methanol was hydrogenated at room temperature for 48 hours. The catalyst was filtered through celite and the volatiles were removed under reduced pressure to afford the title compound in 99% yield. MS (ESI) m/z: 307.3 [M+H]⁺.

### Step 3: tert-butyl 4-(4-((4-chloro-5-(trifluoromethyl)pyrimidin-2-yl)amino)-3-cyclopropyl-1H-pyrazol-1-yl)piperidine-1-carboxylate

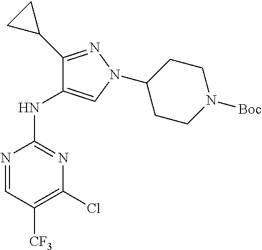 [SMILES: CC(C)(C)OC(=O)N1CCC(n2cc(Nc3ncc(C(F)(F)F)c(Cl)n3)c(C3CC3)n2)CC1]

The title compound was prepared analogously to Example 1, step 5 where 1-(6-amino-7-chloro-1,2,3,4-tetrahydroisoquinolin-2-yl)-2,2,2-trifluoroethan-1-one was replaced with tert-butyl 4-(4-amino-3-cyclopropyl-1H-pyrazol-1-yl)piperidine-1-carboxylate. The title compound was isolated in 27% yield. MS (ESI) m/z: 485.4 [M+H]⁺.

### Step 4: tert-butyl 4-(3-cyclopropyl-4-((4-(4-oxo-4,5,6,7-tetrahydrothieno[3,2-c]pyridin-2-yl)-5-(trifluoromethyl)pyrimidin-2-yl)amino)-1H-pyrazol-1-yl)piperidine-1-carboxylate

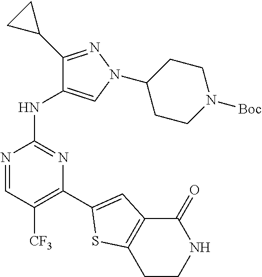 [SMILES: CC(C)(C)OC(=O)N1CCC(n2cc(Nc3ncc(C(F)(F)F)c(-c4cc5c(s4)CCNC5=O)n3)c(C3CC3)n2)CC1]

The title compound was prepared analogously to Example 1, step 6 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one and 4-(methoxycarbonyl)thiophene-2-boronic acid pinacol ester were replaced with tert-butyl 4-(4-((4-chloro-5-(trifluoromethyl)pyrimidin-2-yl)amino)-3-cyclopropyl-1H-pyrazol-1-yl)piperidine-1-carboxylate and 2-(4,4,5,5-tetramethyl-1,3,2-dioxaborolan-2-yl)-4H,5H,6H,7H-thieno[3,2-c]pyridin-4-one. The title compound was isolated in 23% yield. MS (ESI) m/z: 602.3 [M+H]⁺.

### Step 5: 2-(2-((3-cyclopropyl-1-(piperidin-4-yl)-1H-pyrazol-4-yl)amino)-5-(trifluoromethyl)pyrimidin-4-yl)-6,7-dihydrothieno[3,2-c]pyridin-4(5H)-one

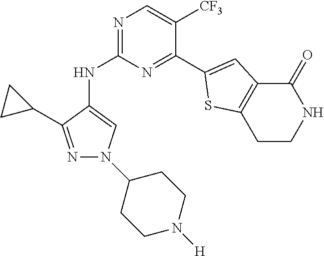 [SMILES: O=C1NCCc2sc(-c3nc(Nc4cn(C5CCNCC5)nc4C4CC4)ncc3C(F)(F)F)cc21]

A solution of tert-butyl 4-(3-cyclopropyl-4-((4-(4-oxo-4,5,6,7-tetrahydrothieno[3,2-c]pyridin-2-yl)-5-(trifluoromethyl)pyrimidin-2-yl)amino)-1H-pyrazol-1-yl)piperidine-1-carboxylate (0.39 mmol) in methanol (3 mL) was treated with a 4M solution of HCl in dioxane (4.8 mL). After 10 minutes, the volatiles were removed under reduced pressure to afford the title compound in 99% yield. MS (ESI) m/z: 504.3 [M+H]⁺.

### Step 6: 2-(2-((3-cyclopropyl-1-(1-methylpiperidin-4-yl)-1H-pyrazol-4-yl)amino)-5-(trifluoromethyl)pyrimidin-4-yl)-6,7-dihydrothieno[3,2-c]pyridin-4(5H)-one

The title compound was prepared analogously to Example 13, step 1 where methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 2-(2-((3-cyclopropyl-1-(piperidin-4-yl)-1H-pyrazol-4-yl)amino)-5-(trifluoromethyl)pyrimidin-4-yl)-6,7-dihydrothieno[3,2-c]pyridin-4(5H)-one. The title compound was isolated in 6% yield. MS (ESI) m/z: 518.2 [M+H]⁺. (400 MHz, DMSO-d₆) δ 9.81 (s, 1H), 9.47 (s, OH), 8.74 (d, J=11.5 Hz, 1H), 8.00 (s, 1H), 7.91-7.71 (m, 2H), 4.00 (s, 1H), 3.50 (td, J=6.8, 2.6 Hz, 2H), 3.15-3.00 (m, 2H), 2.87 (d, J=11.1 Hz, 2H), 2.23 (s, 3H), 2.16-1.80 (m, 7H), 0.84-0.66 (m, 4H).

### Example 49: 2-(2-((6-chloro-2-methyl-1,2,3,4-tetrahydroisoquinolin-7-yl)amino)-5-(trifluoromethyl)pyrimidin-4-yl)-5,6,7,8-tetrahydro-4H-thieno[3,2-c]azepin-4-one

The title compound was prepared analogously to Example 13, step 1 where methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 2-(2-((6-chloro-1,2,3,4-tetrahydroisoquinolin-7-yl)amino)-5-(trifluoromethyl)pyrimidin-4-yl)-5,6,7,8-tetrahydro-4H-thieno[3,2-c]azepin-4-one. The title compound was isolated in 63% yield. MS (ESI) m/z: 508.1 [M+H]⁺. (400 MHz, DMSO-d₆) δ 1.95-2.06 (m, 2H), 2.35 (s, 3H), 2.61 (t, J=5.9 Hz, 2H), 2.84 (t, J=6.0 Hz, 2H), 3.11 (t, J=6.8 Hz, 2H), 3.14-3.19 (m, 2H), 3.49 (s, 2H), 7.23-7.37 (m, 2H), 7.91 (s, 1H), 8.03 (t, J=5.4 Hz, 1H), 8.16 (s, 1H), 8.73 (s, 1H), 9.76 (s, 1H).

### Example 53: 7-(2-((7-chloro-1,2,3,4-tetrahydroisoquinolin-6-yl)amino)-5-(trifluoromethyl)pyrimidin-4-yl)-1,2,3,4-tetrahydro-5H-thieno[2,3-e][1,4]diazepin-5-one

### Step 1: tert-butyl (3-(((1,3-dioxolan-2-yl)methyl)carbamoyl)-5-bromothiophen-2-yl)carbamate

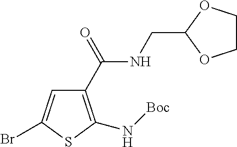 [SMILES: CC(C)(C)OC(=O)Nc1sc(Br)cc1C(=O)NCC1OCCO1]

A solution of 5-bromo-2-{[(tert-butoxy)carbonyl]amino}thiophene-3-carboxylic acid (1.34 mmol), 1-(1,3-dioxolan-2-yl)methanamine (5.21 mmol) and triethylamine (8.69 mmol) in acetonitrile (28 ml) was treated with HCTU (8.69 mmol) and the reaction mixture was stirred at room temperature for 10 minutes, concentrated under vacuum and the resulting residue was suspended in dichloromethane (8 mL). The insoluble materials were filtered off, the filtrate was concentrated and the resulting residue was purified by silica gel chromatography to afford the title compound in 82% yield. MS (ESI) m/z: 408.0 [M+H]⁺.

### Step 2: tert-butyl (5-bromo-3-((2-oxoethyl)carbamoyl)thiophen-2-yl)carbamate

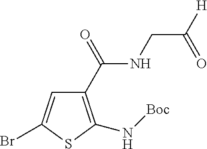 [SMILES: CC(C)(C)OC(=O)Nc1sc(Br)cc1C(=O)NCC=O]

tert-butyl (3-(((1,3-dioxolan-2-yl)methyl)carbamoyl)-5-bromothiophen-2-yl)carbamate (0.88 mmol) was dissolved in methanol (20.0 mL) and 37% methanolic solution of hydrochloric acid (88 mmol). After 2 hours, the reaction mixture was quenched by addition of 1M aqueous sodium hydroxide and the methanol was removed under reduced pressure. The resulting aqueous solution was extracted with dichloromethane three times and the combined organic layers were washed with brine, dried over sodium sulfate, filtered, and concentrated under reduced pressure to afford the title compound in 62% yield.

### Step 3: 7-bromo-1,2,3,4-tetrahydro-5H-thieno[2,3-e][1,4]diazepin-5-one

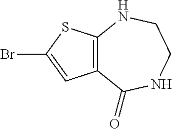 [SMILES: O=C1NCCNc2sc(Br)cc21]

A solution of tert-butyl (5-bromo-3-((2-oxoethyl)carbamoyl)thiophen-2-yl)carbamate (0.28 mmol), acetic acid (0.017 ml) and sodium cyanoborohydride (0.42 mmol) in methanol (3.54 mL) was stirred at 45° C. for 48 hours. The reaction mixture was concentrated under reduced pressure and the residue was purified by silica gel chromatography (1-10% methanol in dichloromethane) to afford the title compound in 25% yield. MS (ESI) m/z: 248.0 [M+H]⁺.

### Step 4: 7-(2-((7-chloro-2-(2,2,2-trifluoroacetyl)-1,2,3,4-tetrahydroisoquinolin-6-yl)amino)-5-(trifluoromethyl)pyrimidin-4-yl)-1,2,3,4-tetrahydro-5H-thieno[2,3-e][1,4]diazepin-5-one

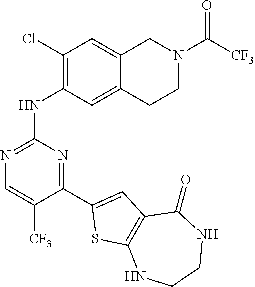 [SMILES: O=C1NCCNc2sc(-c3nc(Nc4cc5c(cc4Cl)CN(C(=O)C(F)(F)F)CC5)ncc3C(F)(F)F)cc21]

The title compound was prepared analogously to Example 4, step 2 where 2-bromo-6,7-dihydrothieno[3,2-C]pyridin-4(5H)-one was replaced with 7-bromo-1,2,3,4-tetrahydro-5H-thieno[2,3-e][1,4]diazepin-5-one. The title compound was isolated in 7% yield. MS (ESI) m/z: 591.0 [M+H]⁺.

### Step 5: 7-(2-((7-chloro-1,2,3,4-tetrahydroisoquinolin-6-yl)amino)-5-(trifluoromethyl)pyrimidin-4-yl)-1,2,3,4-tetrahydro-5H-thieno[2,3-e][1,4]diazepin-5-one

The title compound was prepared analogously to Example 1, step 7 where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 7-(2-((7-chloro-2-(2,2,2-trifluoroacetyl)-1,2,3,4-tetrahydroisoquinolin-6-yl)amino)-5-(trifluoromethyl)pyrimidin-4-yl)-1,2,3,4-tetrahydro-5H-thieno[2,3-e][1,4]diazepin-5-one. The title compound was isolated in 17% yield. MS (ESI) m/z: 495.1 [M+H]⁺. 1H-NMR (400 MHz, DMSO-d₆) δ 2.75 (t, J=5.8 Hz, 2H), 3.00 (t, J=5.9 Hz, 2H), 3.31 (m, 2H), 3.41 (m, 2H), 3.88 (s, 2H), 7.21 (s, 1H), 7.40 (s, 1H), 7.73 (t, J=5.1 Hz, 1H), 7.90 (s, 1H), 8.23 (s, 1H), 8.54 (s, 1H), 8.63 (t, J=3.7 Hz, 1H), 9.34 (s, 1H).

### Example 59: 5-(2-((7-chloro-1,2,3,4-tetrahydroisoquinolin-6-yl)amino)-5-(trifluoromethyl)pyrimidin-4-yl)-3-(methylsulfonyl)thiophene-2-carboxamide

### Step 1: Methyl 3-(methylsulfonyl)thiophene-2-carboxylate

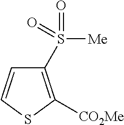 [SMILES: COC(=O)c1sccc1S(C)(=O)=O]

To a solution of sodium bicarbonate (8.73 mmol) in water (5 mL) was added sodium sulfite (8.31 mmol). Methyl 3-chlorosulfonylthiophene-2-carboxylate (4.15 mmol) and ethanol (2.5 mL) were added and the reaction was stirred at 50° C. for 45 minutes. The volatiles were removed under reduced pressure and the resulting residue was dissolved in DMF (7.5 mL) and treated with iodomethane (20.8 mmol). After 15 minutes, the reaction was quenched with water and extracted with ethyl acetate three times. The combined organic layers were washed with brine, dried over anhydrous sodium sulfate, filtered, and concentrated under reduced pressure to afford a residue that was purified by silica gel chromatography (10-30% ethyl acetate in hexanes). The title compound was isolated in 77% yield. MS (ESI) m/z: 221.0[M+H]⁺.

### Step 2: Methyl 5-bromo-3-(methylsulfonyl)thiophene-2-carboxylate

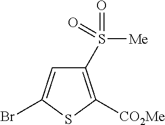 [SMILES: COC(=O)c1sc(Br)cc1S(C)(=O)=O]

1-Bromopyrrolidine-2,5-dione (10.0 mmol) and sulfuric acid (45.4 mmol) were added over a solution of methyl 3-methylsulfonylthiophene-2-carboxylate (4.54 mmol) in acetic acid (10 mL). The mixture was stirred at 60° C. for 12 hours, quenched with water and extracted with ethyl acetate three times. The combined organic layers were washed with brine, dried over anhydrous sodium sulfate, filtered, and concentrated under reduced pressure to afford a residue that was purified by semi-preparative reverse phase-HPLC. The title compound was isolated in 22% yield. MS (ESI) m/z: 299.0[M+H]⁺.

### Step 3: 5-Bromo-3-(methylsulfonyl)thiophene-2-carboxylic acid

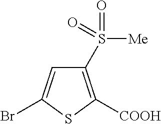 [SMILES: CS(=O)(=O)c1cc(Br)sc1C(=O)O]

To a solution of methyl 5-bromo-3-methylsulfonyl-thiophene-2-carboxylate (1.00 mmol) in methanol (3 mL) and water (0.6 mL) was added sodium hydroxide (2.01 mmol). The mixture was stirred at 25° C. for 2 hours, quenched with water and the pH adjusted to 3 with a 1M aqueous solution of hydrochloric acid. The resulting aqueous solution was extracted with ethyl acetate three times, the combined organic layers were washed with brine, dried over anhydrous sodium sulfate, filtered, and concentrated under reduced pressure to afford the title compound in 91% yield. MS (ESI) m/z: 285.2 [M+H]⁺.

### Step 4: 5-Bromo-3-(methylsulfonyl)thiophene-2-carboxamide

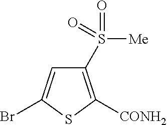 [SMILES: CS(=O)(=O)c1cc(Br)sc1C(N)=O]

A solution of 5-bromo-3-methylsulfonyl-thiophene-2-carboxylic acid (0.10 mmol) in thionyl chloride (1 mL) was stirred at 60° C. for 2 hours. The mixture was concentrated under reduced pressure and the resulting residue was treated with a solution of ammonium hydroxide (1.15 mmol) in THF (1 mL) precooled to 0° C. The mixture was stirred at 25° C. for 1 hour, concentrated and purified by preparative TLC (50% ethyl acetate in hexanes) to afford the title compound in 61% yield. MS (ESI) m/z: 283.8 [M+H]⁺.

### Step 5: 5-(2-((7-chloro-2-(2,2,2-trifluoroacetyl)-1,2,3,4-tetrahydroisoquinolin-6-yl)amino)-5-(trifluoromethyl)pyrimidin-4-yl)-3-(methylsulfonyl)thiophene-2-carboxamide

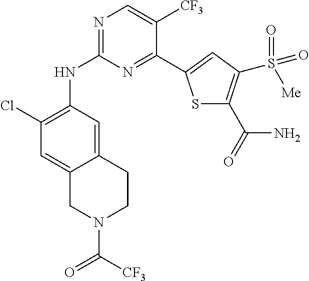 [SMILES: CS(=O)(=O)c1cc(-c2nc(Nc3cc4c(cc3Cl)CN(C(=O)C(F)(F)F)CC4)ncc2C(F)(F)F)sc1C(N)=O]

The title compound was prepared analogously to Example 4, step 2 where 2-bromo-6,7-dihydrothieno[3,2-C]pyridin-4(5H)-one was replaced with 5-bromo-3-(methylsulfonyl)thiophene-2-carboxamide. The title compound was isolated in 22% yield. MS (ESI) m/z: 628.1 [M+H]⁺.

### Step 6: 5-(2-((7-chloro-1,2,3,4-tetrahydroisoquinolin-6-yl)amino)-5-(trifluoromethyl)pyrimidin-4-yl)-3-(methylsulfonyl)thiophene-2-carboxamide

A solution of 5-(2-((7-chloro-2-(2,2,2-trifluoroacetyl)-1,2,3,4-tetrahydroisoquinolin-6-yl)amino)-5-(trifluoromethyl)pyrimidin-4-yl)-3-(methylsulfonyl)thiophene-2-carboxamide (0.03 mmol) in ethanol (1 mL) was treated with a solution of potassium carbonate (0.19 mmol) in water (0.25 mL). After 12 hours, the reaction was concentrated and purified by semi-preparative reverse phase-HPLC to afford the title compound in 31% yield. MS (ESI) m/z: 532.1 [M+H]⁺. ¹H NMR (400 MHz, DMSO-d₆) δ 10.08-9.92 (m, 1H), 8.84 (s, 1H), 8.41 (s, 1H), 8.31-8.17 (m, 1H), 8.08 (s, 1H), 7.88 (s, 1H), 7.32 (s, 1H), 7.26 (s, 1H), 3.89 (s, 3H), 3.43 (s, 2H), 2.98 (s, 2H), 2.71 (s, 2H).

### Example 60: 2-(2-((7-chloro-1,2,3,4-tetrahydroisoquinolin-6-yl)amino)-5-(trifluoromethyl)pyrimidin-4-yl)-6,7-dihydro-5H-thieno[2,3-b][1,4]oxathiepine 4,4-dioxide

### Step 1: 3-(thiophen-3-ylsulfanyl)propan-1-ol

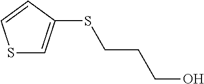

3-bromothiophene (3.00 g, 1.0 eq) was dissolved in diethyl ether (21 mL) and cooled down to −76° C. A 1.6 M solution of n-BuLi in hexanes (12.60 mL) was added dropwise for 10 minutes and after 30 minutes, sulphur (649 mg, 1.10 eq) was added in one portion. After 30 minutes, the reaction was allowed to warm to room temperature during 1 hour. The mixture was cooled down to −30° C. and 3-bromopropanol (2.43 g) was added dropwise. The cooling bath was removed and the reaction was allowed to warm up to room temperature and stirring continued for another 48 hours. Saturated ammonium chloride and ethyl acetate were added and the layers separated. The aqueous layer was extracted with ethyl acetate twice and the combined organic layers were dried over sodium sulfate, filtered, and evaporated to afford the title compound in 60% yield. This crude material was used in the next step without further purification. MS (ESI) m/z: 175.1 [M+H]⁺.

### Step 2: 3-[(2-bromothiophen-3-yl)sulfanyl]propan-1-ol

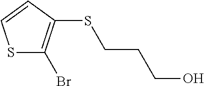 [SMILES: OCCCSc1ccsc1Br]

NBS (2.47 g, 1.05 eq) was added portion wise during 5 minutes to a 0° C. solution of 3-(thiophen-3-ylsulfanyl)propan-1-ol (2.47 g) in dichloromethane (86 mL). After 90 minutes the reaction was diluted with dichloromethane and washed with a half-saturated solution of sodium thiosulfate. The aqueous layer was extracted with dichloromethane twice and the combined organic layers were washed with 1M NaOH and water, dried over sodium sulfate, filtered, and evaporated to afford the title compound in 88% yield. This crude material was used in the next step without further purification. MS (ESI) m/z: 252.9/254.9 [M+H]⁺.

### Step 3: 5H,6H,7H-thieno[2,3-b][1,4]oxathiepine

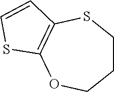

A mixture of 3-(thiophen-3-ylsulfanyl)propan-1-ol (11.3 mmol), cesium carbonate (22.5 mmol), CuI (1.24 mmol) and phenanthroline (2.25 mmol) in toluene (43 mL) was heated at 110° C. for 21 hours. The reaction mixture was cooled down to room temperature, diluted with ethyl acetate, filtered through celite, and eluted with methanol. The volatiles were removed under reduced pressure and the resulting residue was purified by silica gel chromatography (0-5% methanol in dichloromethane). The title compound was isolated in 42% yield. MS (ESI) m/z: 172.7 [M+H]⁺.

### Step 4: 2-bromo-5H,6H,7H-4λ⁶-thieno[2,3-b][1,4]oxathiepine-4,4-dione

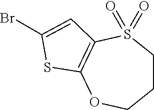 [SMILES: O=S1(=O)CCCOc2sc(Br)cc21]

MCPBA (85 mg) was added to a 0° C. solution of 5H,6H,7H-thieno[2,3-b][1,4]oxathiepine (26 mg) in dichloromethane (1.0 mL). After 10 minutes, the cooling bath was removed and the mixture was stirred at room temperature overnight. The reaction was diluted with saturated NaHCO3 and dichloromethane and the pH of the aqueous layer adjusted to 10 by the addition of 1M aqueous solution of NaOH. The organic layer was separated and the aqueous layer was extracted with dichloromethane twice. The combined organic layers were dried over sodium sulfate, filtered, and concentrated under reduced pressure to afford a residue that was purified by silica gel chromatography (2% ethyl acetate in dichloromethane). The title compound in 51% yield. MS (ESI) m/z: 202.7 [M−H]⁻.

### Step 5: 1-(7-chloro-6-((4-(4,4-dioxido-6,7-dihydro-5H-thieno[2,3-b][1,4]oxathiepin-2-yl)-5-(trifluoromethyl)pyrimidin-2-yl)amino)-3,4-dihydroisoquinolin-2(1H)-yl)-2,2,2-trifluoroethan-1-one

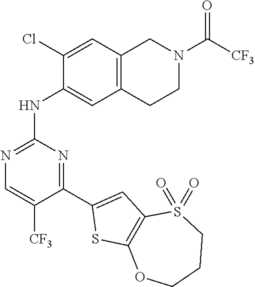 [SMILES: O=C(N1CCc2cc(Nc3ncc(C(F)(F)F)c(-c4cc5c(s4)OCCCS5(=O)=O)n3)c(Cl)cc2C1)C(F)(F)F]

The title compound was prepared analogously to Example 4, step 2 where 2-bromo-6,7-dihydrothieno[3,2-C]pyridin-4(5H)-one was replaced with 2-bromo-5H,6H,7H-4λ⁶-thieno[2,3-b][1,4]oxathiepine-4,4-dione. The title compound was isolated in 24% yield. MS (ESI) m/z: 627.9 [M+H]⁺.

### Step 6: 2-(2-((7-chloro-1,2,3,4-tetrahydroisoquinolin-6-yl)amino)-5-(trifluoromethyl)pyrimidin-4-yl)-6,7-dihydro-5H-thieno[2,3-b][1,4]oxathiepine 4,4-dioxide

The title compound was prepared analogously to Example 1, step 7 where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 1-(7-chloro-6-((4-(4,4-dioxido-6,7-dihydro-5H-thieno[2,3-b][1,4]oxathiepin-2-yl)-5-(trifluoromethyl)pyrimidin-2-yl)amino)-3,4-dihydroisoquinolin-2(1H)-yl)-2,2,2-trifluoroethan-1-one. The title compound was isolated in 45% yield. MS (ESI) m/z: 531.1 [M+H]⁺. 1H-NMR (400 MHz, DMSO-d₆) δ 9.92 (s, 1H), 8.76 (s, 1H), 8.34 (s, 2H), 7.63 (s, 1H), 7.28 (d, J=6.2 Hz, 2H), 4.50 (t, J=5.0 Hz, 2H), 3.92 (s, 2H), 3.68-3.64 (m, 2H), 3.01 (m, 2H), 2.73 (m, 2H), 2.35 (m, 2H).

### Example 63: 5-(2-((7-Chloro-2-methyl-1,2,3,4-tetrahydroisoquinolin-6-yl)amino)-5-(trifluoromethyl)pyrimidin-4-yl)-3-(methylsulfonyl)thiophene-2-carboxamide

The title compound was prepared analogously to Example 13, step 1 where methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 5-(2-((7-chloro-1,2,3,4-tetrahydroisoquinolin-6-yl)amino)-5-(trifluoromethyl)pyrimidin-4-yl)-3-(methylsulfonyl)thiophene-2-carboxamide. The title compound was isolated in 54% yield. MS (ESI) m/z: 546.0 [M+H]⁺. ¹H NMR (400 MHz, DMSO-d₆) δ 10.01 (d, J=3.6 Hz, 1H), 8.84 (s, 1H), 8.41 (s, 1H), 8.20 (s, 1H), 8.07 (s, 1H), 7.88 (s, 1H), 7.34 (s, 1H), 7.28 (d, J=6.4 Hz, 1H), 3.94 (s, 1H), 3.49 (s, 1H), 3.43 (s, 3H), 3.04 (t, J=5.6 Hz, 1H), 2.82 (t, J=5.2 Hz, 1H), 2.75 (t, J=5.2 Hz, 1H), 2.59 (d, J=6.0 Hz, 1H), 2.46-2.33 (m, 3H).

### Example 64: 7-(2-((7-chloro-1,2,3,4-tetrahydroisoquinolin-6-yl)amino)-5-(trifluoromethyl)pyrimidin-4-yl)-2,3,4,5-tetrahydrothieno[2,3-f][1,4]thiazepine 1,1-dioxide

### Step 1: Methyl 3-((2-((tert-butoxycarbonyl)amino)ethyl)sulfonyl)thiophene-2-carboxylate

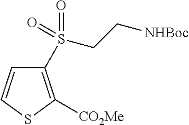 [SMILES: COC(=O)c1sccc1S(=O)(=O)CCNC(=O)OC(C)(C)C]

To a solution of sodium bicarbonate (262 mmol) and sodium sulfite (249 mmol) in water (200 mL) was added methyl 3-chlorosulfonylthiophene-2-carboxylate (125 mmol), methyl 3-chlorosulfonylthiophene-2-carboxylate (125 mmol) and ethyl alcohol (100 mL). The reaction mixture was heated at 50° C. for 45 minutes and concentrated to dryness. The residue was suspended in DMF (300 mL) and tert-butyl-N-(2-bromoethyl)carbamate (249 mmol) was added. After 12 hours, the volatiles were removed under reduced pressure and the residue was treated with water, extracted with ethyl acetate three times and the combined organic layers were dried over anhydrous sodium sulfate, filtered, and concentrated under reduced pressure to afford a residue that was purified by preparative HPLC. The title compound was isolated in 10% yield. MS (ESI) m/z: 250.1 [M+H]⁺.

### Step 2: Methyl 3-((2-aminoethyl)sulfonyl)thiophene-2-carboxylate

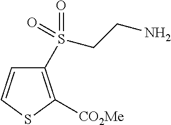 [SMILES: COC(=O)c1sccc1S(=O)(=O)CCN]

A 10° C. solution of methyl 3-((2-((tert-butoxycarbonyl)amino)ethyl)sulfonyl)thiophene-2-carboxylate (11.5 mmol) in dichloromethane (18 mL) was treated with TFA (122 mmol) and the reaction was stirred for 1 hour. Evaporation of volatiles under reduced pressure afforded the title compound, which was used in the next step without further purification. ¹H NMR (400 MHz, DMSO-d₆) δ 8.11-8.10 (m, 1H), 7.58 (d, J=5.2 Hz, 1H), 4.00-3.94 (m, 2H), 3.89 (s, 3H), 3.16-3.06 (m, 2H).

### Step 3: 3,4-Dihydrothieno[2,3-f][1,4]thiazepin-5(2H)-one 1,1-dioxide

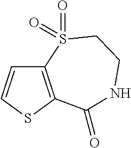 [SMILES: O=C1NCCS(=O)(=O)c2ccsc21]

A mixture of methyl 3-(2-aminoethylsulfonyl)thiophene-2-carboxylate (5.50 mmol) and potassium carbonate (16.5 mmol) in ethyl alcohol (10 mL) was stirred at 70° C. for 12 hours. The reaction mixture was filtered and concentrated under reduced pressure to afford the title compound in 88% yield which was used in the next step without further purification. ¹H NMR (400 MHz, DMSO-d₆) δ 8.77 (brs, 1H), 8.04 (d, J=5.2 Hz, 1H), 7.50 (d, J=5.2 Hz, 1H), 3.88-3.80 (m, 2H), 3.66-3.60 (m, 2H).

### Step 4: 2,3,4,5-Tetrahydrothieno[2,3-f][1,4]thiazepine 1,1-dioxide

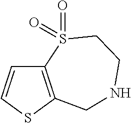 [SMILES: O=S1(=O)CCNCc2sccc21]

A mixture of 1,1-dioxo-3,4-dihydro-2H-thieno[2,3-f][1,4]thiazepin-5-one (4.60 mmol) and borane tetrahydrofuran complex (1 M, 20 mL) was stirred at 70° C. for 12 hours. The reaction was cooled down to 0° C. and methanol was added (10 mL). After 10 minutes, the volatiles were removed under reduced pressure and the crude material was purified by preparative TLC (10% methanol in dichloromethane) to afford the title compound in 60% yield, which was used into the next step without further purification. MS (ESI) m/z: 203.8 [M+H]⁺.

### Step 5: 1-(1,1-Dioxido-2,3-dihydrothieno[2,3-f][1,4]thiazepin-4(5H)-yl)-2,2,2-trifluoroethan-1-one

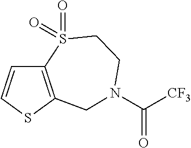 [SMILES: O=C(N1CCS(=O)(=O)c2ccsc2C1)C(F)(F)F]

A 0° C. solution of 2,3,4,5-tetrahydrothieno[2,3-f][1,4]thiazepine 1,1-dioxide (1.67 mmol) and triethylamine (3.35 mmol) in dichloromethane (2 mL) was treated with trifluoroacetic anhydride (2.01 mmol). The mixture was stirred at 10° C. for 1 hour and the pH adjusted to 5 by the addition of ammonium chloride. The solids were filtered off and the solution was concentrated under reduced pressure to afford the title compound, which was used into the next step without further purification. MS (ESI) m/z: 300.0 [M+H]⁺.

### Step 6: 1-(7-bromo-1,1-dioxido-2,3-dihydrothieno[2,3-f][1,4]thiazepin-4(5H)-yl)-2,2,2-trifluoroethan-1-one

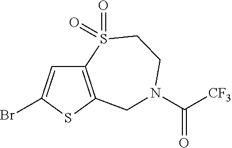 [SMILES: O=C(N1CCS(=O)(=O)c2cc(Br)sc2C1)C(F)(F)F]

A solution of 1-(1,1-dioxido-2,3-dihydrothieno[2,3-f][1,4]thiazepin-4(5H)-yl)-2,2,2-trifluoroethan-1-one (1.40 mmol), N-bromosuccinimide (1.40 mmol) and acetic acid (10 mL) was treated with sulfuric acid (19 mmol) and the mixture was stirred at 60° C. for 12 hours. The pH was brought up to 7 by the addition of sodium bicarbonate, diluted with ethyl acetate and filtered. Evaporation of volatiles under reduced pressure afforded a residue that was purified by preparative TLC (25% ethyl acetate in hexanes) to afford the title compound in 40% yield. MS (ESI) m/z: 378.0 [M+H]⁺.

### Step 7: 1-(7-(2-((7-chloro-2-(2,2,2-trifluoroacetyl)-1,2,3,4-tetrahydroisoquinolin-6-yl)amino)-5-(trifluoromethyl)pyrimidin-4-yl)-1,1-dioxido-2,3-dihydrothieno[2,3-f][1,4]thiazepin-4(5H)-yl)-2,2,2-trifluoroethan-1-one

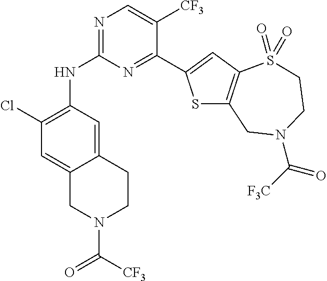 [SMILES: O=C(N1CCc2cc(Nc3ncc(C(F)(F)F)c(-c4cc5c(s4)CN(C(=O)C(F)(F)F)CCS5(=O)=O)n3)c(Cl)cc2C1)C(F)(F)F]

The title compound was prepared analogously to Example 4, step 2, where 2-bromo-6,7-dihydrothieno[3,2-C]pyridin-4(5H)-one was replaced with 1-(7-bromo-1,1-dioxido-2,3-dihydrothieno[2,3-f][1,4]thiazepin-4(5H)-yl)-2,2,2-trifluoroethan-1-one. The title compound was isolated in 87% yield. MS (ESI) m/z: 722.0 [M+H]⁺.

### Step 8: 7-(2-((7-chloro-1,2,3,4-tetrahydroisoquinolin-6-yl)amino)-5-(trifluoromethyl)pyrimidin-4-yl)-2,3,4,5-tetrahydrothieno[2,3-f][1,4]thiazepine 1,1-dioxide

The title compound was prepared analogously to Example 59, step 6 where 5-(2-((7-chloro-2-(2,2,2-trifluoroacetyl)-1,2,3,4-tetrahydroisoquinolin-6-yl)amino)-5-(trifluoromethyl)pyrimidin-4-yl)-3-(methylsulfonyl)thiophene-2-carboxamide was replaced with 1-(7-(2-((7-chloro-2-(2,2,2-trifluoroacetyl)-1,2,3,4-tetrahydroisoquinolin-6-yl)amino)-5-(trifluoromethyl)pyrimidin-4-yl)-1,1-dioxido-2,3-dihydrothieno[2,3-f][1,4]thiazepin-4(5H)-yl)-2,2,2-trifluoroethan-1-one. The title compound was isolated in 54% yield. MS (ESI) m z: 530.0 [M+H]⁺. ¹H NMR (400 MHz, DMSO-d₆) δ 10.03-9.84 (m, 1H), 8.79 (s, 1H), 8.32-8.27 (m, 1H), 7.84-7.79 (m, 1H), 7.35-7.31 (m, 1H), 7.30-7.27 (m, 1H), 4.09 (s, 2H), 3.96 (s, 2H), 3.43-3.39 (m, 2H), 3.37-3.33 (m, 2H), 3.08-3.00 (m, 2H), 2.79-2.73 (m, 2H).

### Example 68: 2-methyl-N-(4-(4-(methylsulfonyl)thiophen-2-yl)-5-(trifluoromethyl)pyrimidin-2-yl)-7-(methylthio)-1,2,3,4-tetrahydroisoquinolin-6-amine

### Step 1: 2,2,2-trifluoro-1-(7-(methylthio)-6-nitro-3,4-dihydroisoquinolin-2(1H)-yl)ethan-1-one

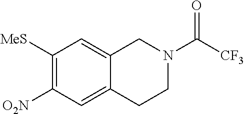 [SMILES: CSc1cc2c(cc1[N+](=O)[O-])CCN(C(=O)C(F)(F)F)C2]

To a solution of 2,2,2-trifluoro-1-[7-chloro-6-nitro-1,2,3,4-tetrahydroisoquinolin-2-yl]ethan-1-one (16 mmol) in dimethyl sulfoxide (100 ml), sodium thiomethoxide (24 mmol) was added in one portion. The reaction mixture was stirred at room temperature for 3 hours, diluted with dichloromethane (200 mL) and treated with a solution of 15 g of K₃PO₄ in 150 ml of water. The aqueous layer was separated and extracted with dichloromethane. The combined organic layers were washed with brine, dried over anhydrous sodium sulfate, and concentrated to afford a crude material that was purified by silica gel chromatography (dichloromethane) to afford the title compound in 62% yield. MS (ESI) m z: 319.5 [M−H]⁻.

### Step 2: 1-(6-amino-7-(methylthio)-3,4-dihydroisoquinolin-2(1H)-yl)-2,2,2-trifluoroethan-1-one

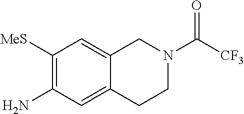 [SMILES: CSc1cc2c(cc1N)CCN(C(=O)C(F)(F)F)C2]

The title compound was prepared analogously to Example 1, step 4 where 1-(7-chloro-6-nitro-1,2,3,4-tetrahydroisoquinolin-2-yl)-2,2,2-trifluoroethan-1-one was replaced with 2,2,2-trifluoro-1-(7-(methylthio)-6-nitro-3,4-dihydroisoquinolin-2(1H)-yl)ethan-1-one. The title compound was isolated in 77% yield. MS (ESI) m z: 289.5 [M−H]⁻.

### Step 3: 1-(6-((4-chloro-5-(trifluoromethyl)pyrimidin-2-yl)amino)-7-(methylthio)-3,4-dihydroisoquinolin-2(1H)-yl)-2,2,2-trifluoroethan-1-one

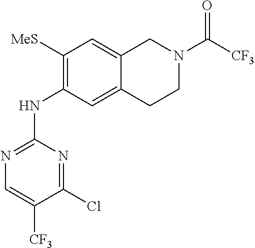 [SMILES: CSc1cc2c(cc1Nc1ncc(C(F)(F)F)c(Cl)n1)CCN(C(=O)C(F)(F)F)C2]

The title compound was prepared analogously to Example 1, step 5 where 1-(6-amino-7-chloro-1,2,3,4-tetrahydroisoquinolin-2-yl)-2,2,2-trifluoroethan-1-one was replaced with 1-(6-amino-7-(methylthio)-3,4-dihydroisoquinolin-2(1H)-yl)-2,2,2-trifluoroethan-1-one. The title compound was isolated in 40% yield. MS (ESI) m z: 469.5 [M−H]⁻.

### Step 4: 2,2,2-trifluoro-1-(6-((4-(4-(methylsulfonyl)thiophen-2-yl)-5-(trifluoromethyl)pyrimidin-2-yl)amino)-7-(methylthio)-3,4-dihydroisoquinolin-2(1H)-yl)ethan-1-one

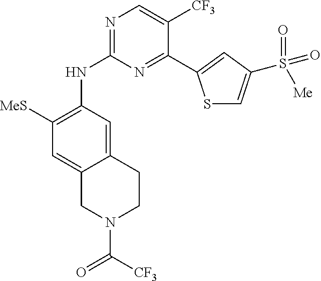 [SMILES: CSc1cc2c(cc1Nc1ncc(C(F)(F)F)c(-c3cc(S(C)(=O)=O)cs3)n1)CCN(C(=O)C(F)(F)F)C2]

The title compound was prepared analogously to Example 1, step 6 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one and 4-(methoxycarbonyl)thiophene-2-boronic acid pinacol ester were replaced with 1-(6-{[4-chloro-5-(trifluoromethyl)pyrimidin-2-yl]amino}-7-(methylsulfanyl)-1,2,3,4-tetrahydroisoquinolin-2-yl)-2,2,2-trifluoroethan-1-one and 2-(4-methanesulfonylthiophen-2-yl)-4,4,5,5-tetramethyl-1,3,2-dioxaborolane. The title compound was isolated in 29% yield. MS (ESI) m z: 596.9 [M+H]⁺.

### Step 5: N-(4-(4-(methylsulfonyl)thiophen-2-yl)-5-(trifluoromethyl)pyrimidin-2-yl)-7-(methylthio)-1,2,3,4-tetrahydroisoquinolin-6-amine

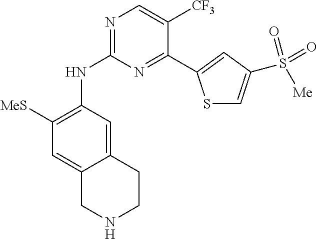 [SMILES: CSc1cc2c(cc1Nc1ncc(C(F)(F)F)c(-c3cc(S(C)(=O)=O)cs3)n1)CCNC2]

The title compound was prepared analogously to Example 1, step 7 where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 2,2,2-trifluoro-1-(6-((4-(4-(methylsulfonyl)thiophen-2-yl)-5-(trifluoromethyl)pyrimidin-2-yl)amino)-7-(methylthio)-3,4-dihydroisoquinolin-2(1H)-yl)ethan-1-one. The title compound was isolated in 98% yield. MS (ESI) m z: 501.0 [M+H]⁺.

### Step 6: 2-methyl-N-(4-(4-(methylsulfonyl)thiophen-2-yl)-5-(trifluoromethyl)pyrimidin-2-yl)-7-(methylthio)-1,2,3,4-tetrahydroisoquinolin-6-amine

The title compound was prepared analogously to Example 13, step 1 where methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with N-(4-(4-(methylsulfonyl)thiophen-2-yl)-5-(trifluoromethyl)pyrimidin-2-yl)-7-(methylthio)-1,2,3,4-tetrahydroisoquinolin-6-amine. The title compound was isolated in 31% yield. MS (ESI) m z: 515.1 [M+H]⁺. 1H-NMR (300 MHz, DMSO-d₆) δ 2.36 (s, 6H), 2.60 (t, J=5.9 Hz, 2H), 2.80 (t, J=5.9 Hz, 2H), 3.28 (s, 3H), 3.52 (s, 2H), 7.08 (s, 1H), 7.16 (s, 1H), 7.87 (s, 1H), 8.21 (s, 1H), 8.64 (d, J=1.4 Hz, 1H), 8.77 (s, 1H), 9.74 (s, 1H).

### Example 69: 7-(2-((7-chloro-2-methyl-1,2,3,4-tetrahydroisoquinolin-6-yl)amino)-5-(trifluoromethyl)pyrimidin-4-yl)-2,3,4,5-tetrahydrothieno[2,3-f][1,4]thiazepine 1,1-dioxide

### Step 1: 7-bromo-2,3,4,5-tetrahydrothieno[2,3-f][1,4]thiazepine 1,1-dioxide

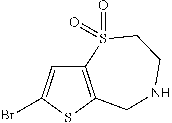 [SMILES: O=S1(=O)CCNCc2sc(Br)cc21]

The title compound was prepared analogously to Example 59, step 6, where 5-(2-((7-chloro-2-(2,2,2-trifluoroacetyl)-1,2,3,4-tetrahydroisoquinolin-6-yl)amino)-5-(trifluoromethyl)pyrimidin-4-yl)-3-(methylsulfonyl)thiophene-2-carboxamide was replaced with 1-(7-bromo-1,1-dioxido-2,3-dihydrothieno[2,3-f][1,4]thiazepin-4(5H)-yl)-2,2,2-trifluoroethan-1-one. The title compound was isolated. MS (ESI) m z: 284.0 [M+H]⁺.

### Step 2: tert-butyl 7-bromo-2,3-dihydrothieno[2,3-f][1,4]thiazepine-4(5H)-carboxylate 1,1-dioxide

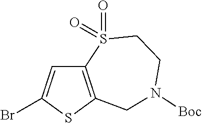 [SMILES: CC(C)(C)OC(=O)N1CCS(=O)(=O)c2cc(Br)sc2C1]

A 0° C. solution of 7-bromo-2,3,4,5-tetrahydrothieno[2,3-f][1,4]thiazepine 1,1-dioxide (2.0 mmol), trimethylamine (4.0 mmol) in dichloromethane (6 mL) was treated with di-tert-butyldicarbonate (2.60 mmol). The reaction mixture was stirred at 10° C. for 2 hours, quenched with water and extracted with ethyl acetate three times. The combined organic layers were washed with saturated brine, dried over anhydrous sodium sulfate, filtered, and concentrated under reduced pressure to afford a crude material that was purified by preparative TLC (25% ethyl acetate in hexanes). The title compound was isolated in 67% yield. ¹H NMR (400 MHz, DMSO-d₆) δ 7.49-7.39 (m, 1H), 4.68-4.57 (m, 2H), 3.97-3.88 (m, 2H), 3.64-3.49 (m, 2H), 1.36 (s, 9H).

### Step 3: tert-butyl 7-(2-((7-chloro-2-(2,2,2-trifluoroacetyl)-1,2,3,4-tetrahydroisoquinolin-6-yl)amino)-5-(trifluoromethyl)pyrimidin-4-yl)-2,3-dihydrothieno[2,3-f][1,4]thiazepine-4(5H)-carboxylate 1,1-dioxide

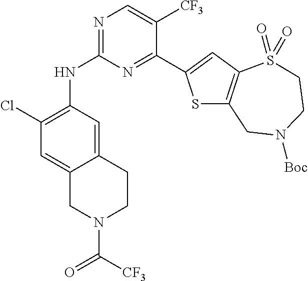 [SMILES: CC(C)(C)OC(=O)N1CCS(=O)(=O)c2cc(-c3nc(Nc4cc5c(cc4Cl)CN(C(=O)C(F)(F)F)CC5)ncc3C(F)(F)F)sc2C1]

The title compound was prepared analogously to Example 4, step 2 where 2-bromo-6,7-dihydrothieno[3,2-C]pyridin-4(5H)-one were replaced with tert-butyl 7-bromo-2,3-dihydrothieno[2,3-f][1,4]thiazepine-4(5H)-carboxylate 1,1-dioxide. The title compound was isolated in 68% yield. MS (ESI) m/z: 726.0 [M+H]⁺.

### Step 4: tert-butyl 7-(2-((7-chloro-1,2,3,4-tetrahydroisoquinolin-6-yl)amino)-5-(trifluoromethyl)pyrimidin-4-yl)-2,3-dihydrothieno[2,3-f][1,4]thiazepine-4(5H)-carboxylate 1,1-dioxide

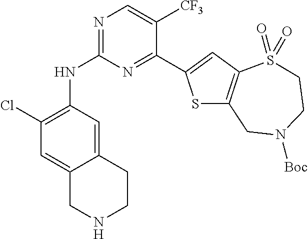 [SMILES: CC(C)(C)OC(=O)N1CCS(=O)(=O)c2cc(-c3nc(Nc4cc5c(cc4Cl)CNCC5)ncc3C(F)(F)F)sc2C1]

The title compound was prepared analogously to Example 59, step 6 where 5-(2-((7-chloro-2-(2,2,2-trifluoroacetyl)-1,2,3,4-tetrahydroisoquinolin-6-yl)amino)-5-(trifluoromethyl)pyrimidin-4-yl)-3-(methylsulfonyl)thiophene-2-carboxamide was replaced with tert-butyl 7-(2-((7-chloro-2-(2,2,2-trifluoroacetyl)-1,2,3,4-tetrahydroisoquinolin-6-yl)amino)-5-(trifluoromethyl)pyrimidin-4-yl)-2,3-dihydrothieno[2,3-f][1,4]thiazepine-4(5H)-carboxylate 1,1-dioxide. The title compound was isolated in 87% yield. MS (ESI) m/z: 630.1 [M+H]⁺.

### Step 5: tert-butyl 7-(2-((7-chloro-2-methyl-1,2,3,4-tetrahydroisoquinolin-6-yl)amino)-5-(trifluoromethyl)pyrimidin-4-yl)-2,3-dihydrothieno[2,3-f][1,4]thiazepine-4(5H)-carboxylate 1,1-dioxide

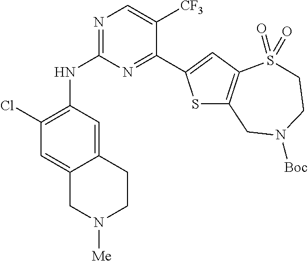 [SMILES: CN1CCc2cc(Nc3ncc(C(F)(F)F)c(-c4cc5c(s4)CN(C(=O)OC(C)(C)C)CCS5(=O)=O)n3)c(Cl)cc2C1]

The title compound was prepared analogously to Example 13, step 1 where methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with tert-butyl 7-(2-((7-chloro-1,2,3,4-tetrahydroisoquinolin-6-yl)amino)-5-(trifluoromethyl)pyrimidin-4-yl)-2,3-dihydrothieno[2,3-f][1,4]thiazepine-4(5H)-carboxylate 1,1-dioxide. The title compound was isolated in 88% yield. MS (ESI) m/z: 644.0 [M+H]⁺.

### Step 6: 7-(2-((7-chloro-2-methyl-1,2,3,4-tetrahydroisoquinolin-6-yl)amino)-5-(trifluoromethyl)pyrimidin-4-yl)-2,3,4,5-tetrahydrothieno[2,3-f][1,4]thiazepine 1,1-dioxide

The title compound was prepared analogously to Example 64, step 2 where methyl 3-((2-((tert-butoxycarbonyl)amino)ethyl)sulfonyl)thiophene-2-carboxylate was replaced with tert-butyl 7-(2-((7-chloro-2-methyl-1,2,3,4-tetrahydroisoquinolin-6-yl)amino)-5-(trifluoromethyl)pyrimidin-4-yl)-2,3-dihydrothieno[2,3-f][1,4]thiazepine-4(5H)-carboxylate 1,1-dioxide. The title compound was isolated in 31% yield. MS (ESI) m/z: 544.0 [M+H]⁺. ¹H NMR (400 MHz, CDCl₃) δ 8.75-8.70 (m, 1H), 8.16-8.09 (m, 2H), 7.77-7.71 (m, 1H), 7.14-7.08 (m, 1H), 4.31-4.27 (m, 2H), 3.67-3.63 (m, 2H), 3.60-3.55 (m, 2H), 3.34-3.30 (m, 2H), 3.02-2.96 (m, 2H), 2.78-2.73 (m, 2H), 2.54-2.46 (m, 3H).

### Example 71: 7-(2-((7-chloro-1,2,3,4-tetrahydroisoquinolin-6-yl)amino)-5-(trifluoromethyl)pyrimidin-4-yl)-3,4-dihydrothieno[3,2-f][1,4]oxazepin-5(2H)-one

### Step 1: 2-(2-hydroxyethoxy)thiophene-3-carboxylic acid

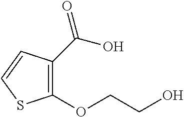 [SMILES: O=C(O)c1ccsc1OCCO]

Potassium tert-butoxide (72.4 mmol) was dissolved in ethylene glycol (11 mL) and copper(I) iodide (2.42 mmol) and 2-bromo-3-thiophenecarboxylic acid (24 mmol) were added portion-wise. The reaction mixture was stirred at 120° C. for 3 hours. The mixture was cooled down to room temperature and the pH made neutral by the addition of formic acid. The solution was diluted with methanol and filtered through celite. The volatiles were removed under reduced pressure and the crude material was purified by silica gel chromatography (0-5% methanol in dichloromethane). The title compound was isolated in 15% yield. MS (ESI) m/z: 189.3 [M+H]⁺.

### Step 2: 2-(2-hydroxyethoxy)thiophene-3-carboxamide

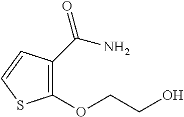 [SMILES: NC(=O)c1ccsc1OCCO]

A solution of 2-(2-hydroxyethoxy)thiophene-3-carboxylic acid (4.67 mmol), triethylamine (26.3 mmol) and HCTU (14.0 mmol) in DMF (13 ml) was treated with a 0.4 M solution of ammonia in dioxane (22.7 mmol). After 1 hour, a second portion of HCTU (14.0 mmol) and 0.4 M solution of ammonia in dioxane (22.7 mmol) was added and stirring continued overnight. The reaction mixture was concentrated under reduced pressure and the crude material was purified by silica gel chromatography (5-10% dichloromethane in methanol) to afford the title compound in 95% yield. MS (ESI) m/z: 188.0 [M+H]⁺.

### Step 3: 2-((3-carbamoylthiophen-2-yl)oxy)ethyl methanesulfonate

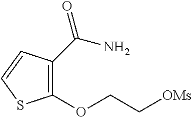 [SMILES: CS(=O)(=O)OCCOc1sccc1C(N)=O]

A solution of 2-(2-hydroxyethoxy)thiophene-3-carboxamide (2.67 mmol) and DIPEA (3.73 mmol) in dichloromethane (7.5 ml) was cooled to 0° C. and methanesulfonyl chloride (3.20 mmol) was added dropwise. The reaction mixture was stirred at room temperature for 1 hour. 0.4 additional equivalents of DIPEA and 0.2 additional equivalents of methanesulfonyl chloride were added and stirring continued for another hour. The reaction was quenched with water and the organic layer was separated, washed with brine, dried over anhydrous sodium sulfate, and filtered. Evaporation of volatiles under reduced pressure afforded the title compound in 910% yield. This material was used in the following step without further purification. MS (ESI) m/z: 266.0 [M+H]⁺.

### Step 4: 3,4-dihydrothieno[3,2-f][1,4]oxazepin-5(2H)-one

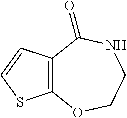 [SMILES: O=C1NCCOc2sccc21]

60% Sodium hydride (2.92 mmol) was added to a solution of 2-((3-carbamoylthiophen-2-yl)oxy)ethyl methanesulfonate (2.44 mmol) in DMF (14 mL). After 12 hours, the reaction was acidified by the addition of acetic acid (0.07 mL) and the volatiles were removed under reduced pressure. The crude material was purified by silica gel chromatography to afford the title compound in 66% yield. MS (ESI) m/z: 169.9 [M+H]⁺.

### Step 5: 7-bromo-3,4-dihydrothieno[3,2-f][1,4]oxazepin-5(2H)-one

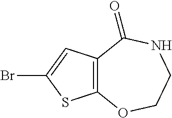 [SMILES: O=C1NCCOc2sc(Br)cc21]

NBS (1.52 mmol) was added to a solution of 3,4-dihydrothieno[3,2-f][1,4]oxazepin-5(2H)-one (1.60 mmol) in dichloromethane (0.3 mL). After 1 hour, the reaction mixture was concentrated under reduced pressure and the residue purified by silica gel chromatography (0-10% methanol in 1M solution of ammonia in methanol). The title compound was isolated in 87% yield. MS (ESI) m/z: 250.4 [M+H]⁺.

### Step 6: 7-(2-((7-chloro-2-(2,2,2-trifluoroacetyl)-1,2,3,4-tetrahydroisoquinolin-6-yl)amino)-5-(trifluoromethyl)pyrimidin-4-yl)-3,4-dihydrothieno[3,2-f][1,4]oxazepin-5(2H)-one

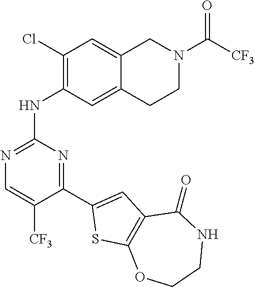 [SMILES: O=C1NCCOc2sc(-c3nc(Nc4cc5c(cc4Cl)CN(C(=O)C(F)(F)F)CC5)ncc3C(F)(F)F)cc21]

The title compound was prepared analogously to Example 4, step 2 where 2-bromo-6,7-dihydrothieno[3,2-C]pyridin-4(5H)-one was replaced with 7-bromo-3,4-dihydrothieno[3,2-f][1,4]oxazepin-5(2H)-one. The title compound was isolated in 48% yield. MS (ESI) m/z: 592.6 [M+H]⁺.

### Step 7: 7-(2-((7-chloro-1,2,3,4-tetrahydroisoquinolin-6-yl)amino)-5-(trifluoromethyl)pyrimidin-4-yl)-3,4-dihydrothieno[3,2-f][1,4]oxazepin-5(2H)-one

The title compound was prepared analogously to Example 1, step 7 where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 7-(2-((7-chloro-2-(2,2,2-trifluoroacetyl)-1,2,3,4-tetrahydroisoquinolin-6-yl)amino)-5-(trifluoromethyl)pyrimidin-4-yl)-3,4-dihydrothieno[3,2-f][1,4]oxazepin-5(2H)-one. The title compound was isolated in 90% yield. MS (ESI) m/z: 496.4 [M+H]⁺. 1H-NMR (300 MHz, DMSO-d₆) δ 9.72 (s, 1H), 8.68 (s, 1H), 8.18 (m, 1H), 7.88 (s, 1H), 7.30 (m, 1H), 7.22 (s, 1H), 4.53 (m, 2H), 3.86 (s, 2H), 3.49 (m, 2H), 2.96 (m, 2H), 2.69 (m, 2H).

### Example 72: 6-ethyl-N-(4-(4-(methylsulfonyl)thiophen-2-yl)-5-(trifluoromethyl)pyrimidin-2-yl)isoindolin-5-amine

### Step 1: 1-(5-bromoisoindolin-2-yl)-2,2,2-trifluoroethan-1-one

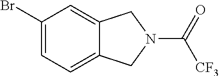 [SMILES: O=C(N1Cc2ccc(Br)cc2C1)C(F)(F)F]

Triethylamine (76 mmol) was added to a solution of 5-bromo-2,3-dihydro-1H-isoindole (50 mmol) in dichloromethane (120 mL). The resulting solution was cooled down to 0° C. and trifluoroacetic anhydride (56 mmol) were added dropwise maintaining the temperature of the reaction mixture below 5° C. After 2 hours, the reaction was quenched with water, the organic layer was separated and washed with water, dried over anhydrous sodium sulfate, filtered, and concentrated to afford a residue that was purified by silica gel chromatography (30% ethyl acetate in hexanes). The title compound was isolated in 75% yield. 1H-NMR (300 MHz, DMSO-d₆) δ 7.63 (dd, J=7.6, 1.7 Hz, 1H), 7.53 (dd, J=8.2, 1.9 Hz, 1H), 7.36 (dd, J=8.1, 6.3 Hz, 1H), 5.01 (d, J=12.0 Hz, 2H), 4.81 (d, J=12.8 Hz, 2H).

### Step 2: 1-(5-ethylisoindolin-2-yl)-2,2,2-trifluoroethan-1-one

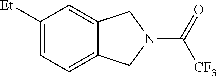 [SMILES: CCc1ccc2c(c1)CN(C(=O)C(F)(F)F)C2]

A mixture of 1-(5-bromo-2,3-dihydro-1H-isoindol-2-yl)-2,2,2-trifluoroethan-1-one (18 mmol), 1,1′-bis(diphenylphosphino)ferrocene (1.4 mmol), ethylboronic acid (35 mmol), potassium phosphate tribasic (53 mmol), 1,1′-Bis(diphenylphosphino)ferrocene-palladium(II)dichloride dichloromethane (0.88 mmol) in toluene (104 mL) and water (16 ml) was stirred at 80° C. for 4 hours. The reaction mixture was cooled down to room temperature and washed with water. The organic layer was concentrated and the resulting crude material was purified by silica gel chromatography (dichloromethane) to afford the title compound in 89% yield. MS (ESI) m/z: 244.0 [M−H]⁻.

### Step 3: 1-(5-ethyl-6-nitroisoindolin-2-yl)-2,2,2-trifluoroethan-1-one

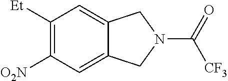 [SMILES: CCc1cc2c(cc1[N+](=O)[O-])CN(C(=O)C(F)(F)F)C2]

A solution of 1-(5-ethyl-2,3-dihydro-1H-isoindol-2-yl)-2,2,2-trifluoroethan-1-one (16 mmol) in acetic anhydride (22 ml) was cooled down to −30° C. Nitric acid (0.83 mL) was added slowly over 10 minutes. The reaction mixture warmed up to room temperature and stirred for 2 hours. Methanol (15 mL) was added and the mixture stirred for another 30 minutes. Evaporation of the volatiles under reduced pressure afforded a residue that was partitioned between water and dichloromethane. The organic layer was separated, washed with saturated sodium bicarbonate, dried over anhydrous sodium sulfate, and concentrated under reduced pressure. The crude product was purified by silica gel chromatography (0-30% ethyl acetate in hexanes). The title compound was isolated in 45% yield. 1H-NMR (300 MHz, CDCl₃) δ 1.30 (m, 3H), 2.94 (m, 2H), 4.95 (s, 2H), 5.07 (s, 2H), 7.32 (d, J=15.5 Hz, 1H), 7.84 (d, J=12.3 Hz, 1H).

### Step 4: 1-(5-amino-6-ethylisoindolin-2-yl)-2,2,2-trifluoroethan-1-one

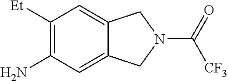 [SMILES: CCc1cc2c(cc1N)CN(C(=O)C(F)(F)F)C2]

A solution of 1-(5-ethyl-6-nitroisoindolin-2-yl)-2,2,2-trifluoroethan-1-one (7.14 mmol) in methanol (42 mL) was hydrogenated in the presence of 10% palladium on carbon (0.21 mmol) for 12 hours. The mixture was filtered through celite and the methanol was evaporated. The crude material was purified by silica gel chromatography (20-60% ethyl acetate in hexanes) to afford the title compound in 55% yield. MS (ESI) m/z: 257.6 [M−H]⁻.

### Step 5: 1-(5-((4-chloro-5-(trifluoromethyl)pyrimidin-2-yl)amino)-6-ethylisoindolin-2-yl)-2,2,2-trifluoroethan-1-one

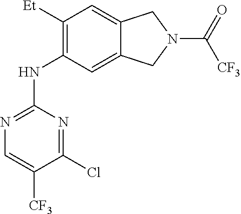 [SMILES: CCc1cc2c(cc1Nc1ncc(C(F)(F)F)c(Cl)n1)CN(C(=O)C(F)(F)F)C2]

The title compound was prepared analogously to Example 1, step 5, where 1-(6-amino-7-chloro-1,2,3,4-tetrahydroisoquinolin-2-yl)-2,2,2-trifluoroethan-1-one was replaced with 1-(5-amino-6-ethylisoindolin-2-yl)-2,2,2-trifluoroethan-1-one. The title compound was isolated in 36% yield. MS (ESI) m/z: 439.0 [M+H]⁺.

### Step 6: 1-(5-ethyl-6-((4-(4-(methylsulfonyl)thiophen-2-yl)-5-(trifluoromethyl)pyrimidin-2-yl)amino)isoindolin-2-yl)-2,2,2-trifluoroethan-1-one

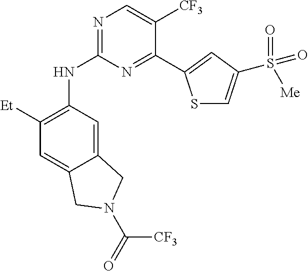 [SMILES: CCc1cc2c(cc1Nc1ncc(C(F)(F)F)c(-c3cc(S(C)(=O)=O)cs3)n1)CN(C(=O)C(F)(F)F)C2]

The title compound was prepared analogously to Example 1, step 6, where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one and 4-(methoxycarbonyl)thiophene-2-boronic acid pinacol ester were replaced with 1-(5-((4-chloro-5-(trifluoromethyl)pyrimidin-2-yl)amino)-6-ethylisoindolin-2-yl)-2,2,2-trifluoroethan-1-one and 2-(4-methanesulfonylthiophen-2-yl)-4,4,5,5-tetramethyl-1,3,2-dioxaborolane. The title compound was isolated in 8% yield. MS (ESI) m/z: 565.0 [M+H]⁺.

### Step 7: 6-ethyl-N-(4-(4-(methylsulfonyl)thiophen-2-yl)-5-(trifluoromethyl)pyrimidin-2-yl)isoindolin-5-amine

The title compound was prepared analogously to Example 1, step 7, where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 1-(5-ethyl-6-((4-(4-(methylsulfonyl)thiophen-2-yl)-5-(trifluoromethyl)pyrimidin-2-yl)amino)isoindolin-2-yl)-2,2,2-trifluoroethan-1-one. The title compound was isolated in 15% yield. MS (ESI) m/z: 469.1 [M+H]⁺. 1H-NMR (400 MHz, DMSO-d₆) δ 1.09 (t, J=7.5 Hz, 3H), 2.59 (q, J=7.5 Hz, 2H), 3.28 (s, 3H), 4.19 (s, 4H), 7.23 (s, 1H), 7.26 (s, 1H), 7.87 (s, 1H), 8.33 (s, 2H), 8.63 (s, 1H), 8.76 (s, 1H), 9.83 (s, 1H).

### Example 73: 6-chloro-N-(4-(4-(methylsulfonyl)thiophen-2-yl)-5-(trifluoromethyl)pyrimidin-2-yl)isoindolin-5-amine

The title compound was prepared analogously to Example 72, where 5-bromo-2,3-dihydro-1H-isoindole was replaced with 5-chloro-2,3-dihydro-1H-isoindole in step 1. MS (ESI) m/z: 473.2 [M−H]⁻. 1H-NMR (400 MHz, DMSO-d₆) δ 10.04 (s, 1H), 8.82 (s, 1H), 8.66 (d, J=1.4 Hz, 1H), 8.22 (s, 1H), 7.89 (s, 1H), 7.48 (d, J=5.3 Hz, 2H), 4.15 (s, 4H), 3.29 (s, 3H).

### Example 75: 7-chloro-N-(4-(5-(3,6-dihydro-2H-pyran-4-yl)-4-(methylsulfonyl)thiophen-2-yl)-5-(trifluoromethyl)pyrimidin-2-yl)-1,2,3,4-tetrahydroisoquinolin-6-amine

### Step 1: 2,5-dibromo-3-(methylthio)thiophene

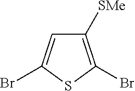

To a solution of 3-methylsulfanylthiophene (38 mmol) in dichloromethane (50 mL) was added 1-bromopyrrolidine-2,5-dione (84 mmol). After 12 hours the volatiles were removed under reduced pressure and the resulting residue was purified by silica gel chromatography (0-10% ethyl acetate in petroleum ether). The title compound was isolated in 74% yield. ¹H NMR (400 MHz, CDCl₃) δ 6.90 (s, 1H), 2.45 (s, 3H).

### Step 2: 2,5-dibromo-3-(methylsulfonyl)thiophene

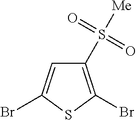 [SMILES: CS(=O)(=O)c1cc(Br)sc1Br]

To a 0° C. solution of 2,5-dibromo-3-methylsulfanyl-thiophene (14 mmol) in dichloromethane (40 mL) was added meta-chloroperoxybenzoic acid (28 mmol). The mixture was stirred at 25° C. for 2 hours and quenched by the addition of saturated aqueous solution of sodium sulfite (100 mL). The mixture was extracted with dichloromethane three times and the combined organic layers were washed with brine, dried over anhydrous sodium sulfate, filtered, and concentrated under reduced pressure to afford a residue that was purified by silica gel chromatography (10-50% ethyl acetate in petroleum ether). The title compound was isolated in 74% yield. ¹H NMR (400 MHz, CDCl₃) δ 7.38 (s, 1H), 3.18 (s, 3H).

### Step 3: 4-(5-bromo-3-(methylsulfonyl)thiophen-2-yl)-3,6-dihydro-2H-pyran

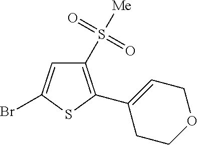 [SMILES: CS(=O)(=O)c1cc(Br)sc1C1=CCOCC1]

A mixture of 2,5-dibromo-3-methylsulfonyl-thiophene (0.62 mmol), 2-(3,6-dihydro-2H-pyran-4-yl)-4,4,5,5-tetramethyl-1,3,2-dioxaborolane (0.62 mmol), potassium phosphate (1.87 mmol) and tetrakis[triphenylphosphine]palladium(0) (0.06 mmol) in dioxane (2 mL) and water (0.4 mL) was stirred at 80° C. for 1 hour. The mixture was purified by preparative TLC (33% ethyl acetate in petroleum ether, Rf=0.32). The title compound was isolated in 59% yield. ¹H NMR (400 MHz, CDCl₃) δ 7.37 (s, 1H), 6.24-6.21 (m, 1H), 4.31 (q, J=2.8 Hz, 2H), 3.91 (t, J=5.2 Hz, 2H), 3.08 (s, 3H), 2.52 (dt, J=2.4, 4.8 Hz, 2H).

### Step 4: 1-(7-chloro-6-((4-(5-(3,6-dihydro-2H-pyran-4-yl)-4-(methylsulfonyl)thiophen-2-yl)-5-(trifluoromethyl)pyrimidin-2-yl)amino)-3,4-dihydroisoquinolin-2(1H)-yl)-2,2,2-trifluoroethan-1-one

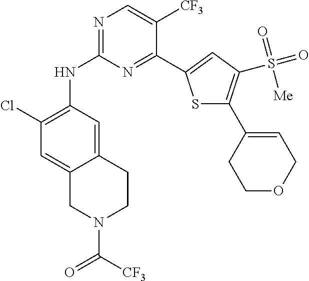 [SMILES: CS(=O)(=O)c1cc(-c2nc(Nc3cc4c(cc3Cl)CN(C(=O)C(F)(F)F)CC4)ncc2C(F)(F)F)sc1C1=CCOCC1]

The title compound was prepared analogously to Example 4, step 2 where 2-bromo-6,7-dihydrothieno[3,2-C]pyridin-4(5H)-one was replaced with 4-(5-bromo-3-(methylsulfonyl)thiophen-2-yl)-3,6-dihydro-2H-pyran. The title compound was isolated in 62% yield. MS (ESI) m/z: 667.0 [M+H]⁺

### Step 5: 7-chloro-N-(4-(5-(3,6-dihydro-2H-pyran-4-yl)-4-(methylsulfonyl)thiophen-2-yl)-5-(trifluoromethyl)pyrimidin-2-yl)-1,2,3,4-tetrahydroisoquinolin-6-amine

The title compound was prepared analogously to Example 59, step 6 where 5-(2-((7-chloro-2-(2,2,2-trifluoroacetyl)-1,2,3,4-tetrahydroisoquinolin-6-yl)amino)-5-(trifluoromethyl)pyrimidin-4-yl)-3-(methylsulfonyl)thiophene-2-carboxamide was replaced with 1-(7-chloro-6-((4-(5-(3,6-dihydro-2H-pyran-4-yl)-4-(methylsulfonyl)thiophen-2-yl)-5-(trifluoromethyl)pyrimidin-2-yl)amino)-3,4-dihydroisoquinolin-2(1H)-yl)-2,2,2-trifluoroethan-1-one. The title compound was isolated in 42% yield.

MS (ESI) m/z: 571.1 [M+H]⁺. ¹H NMR (400 MHz, DMSO-d₆) δ 9.91 (d, J=2.0 Hz, 1H), 8.80 (s, 1H), 8.24 (s, 1H), 7.89 (s, 1H), 7.34 (s, 1H), 7.25 (s, 1H), 6.36 (s, 1H), 4.24 (d, J=2.8 Hz, 2H), 3.88 (s, 2H), 3.81 (t, J=5.2 Hz, 2H), 3.26 (s, 3H), 2.98 (t, J=5.6 Hz, 2H), 2.70 (t, J=5.6 Hz, 2H), 2.53-2.51 (m, 2H).

### Example 77: 6-chloro-2-methyl-N-(4-(4-(methylsulfonyl)thiophen-2-yl)-5-(trifluoromethyl)pyrimidin-2-yl)isoindolin-5-amine

The title compound was prepared analogously to Example 13, step 1 where methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 6-chloro-N-(4-(4-(methylsulfonyl)thiophen-2-yl)-5-(trifluoromethyl)pyrimidin-2-yl)isoindolin-5-amine. The title compound was isolated in 21% yield. MS (ESI) m/z: 487.1 [M+H]⁺. 1H-NMR (400 MHz, DMSO-d₆) δ 10.01 (s, 1H), 8.83 (s, 1H), 8.66 (d, J=1.4 Hz, 1H), 8.24 (s, 1H), 7.89 (s, 1H), 7.44 (s, 2H), 3.82 (d, J=4.2 Hz, 3H), 2.49 (s, 3H).

### Example 80: 6-fluoro-N-[4-(4-methanesulfonylthiophen-2-yl)-5-(trifluoromethyl)pyrimidin-2-yl]-2-methyl-2,3-dihydro-1H-isoindol-5-amine

### Step 1: 1-(5-amino-6-fluoro-2,3-dihydro-1H-isoindol-2-yl)-2,2,2-trifluoroethan-1-one

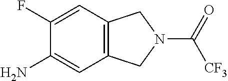 [SMILES: Nc1cc2c(cc1F)CN(C(=O)C(F)(F)F)C2]

The title compound was prepared analogously to Example 72 following steps 1, 3 and 4, where 5-bromo-2,3-dihydro-1H-isoindole was replaced with 5-fluoro-2,3-dihydro-1H-isoindole in step 1. MS (ESI) m/z: 249.0 [M+H]⁺.

### Step 2: 1-(5-((4-chloro-5-(trifluoromethyl)pyrimidin-2-yl)amino)-6-fluoroisoindolin-2-yl)-2,2,2-trifluoroethan-1-one

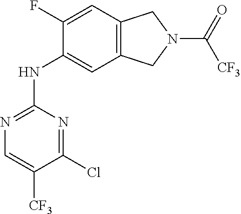 [SMILES: O=C(N1Cc2cc(F)c(Nc3ncc(C(F)(F)F)c(Cl)n3)cc2C1)C(F)(F)F]

The title compound was prepared analogously to Example 1, step 5 where 1-(6-amino-7-chloro-1,2,3,4-tetrahydroisoquinolin-2-yl)-2,2,2-trifluoroethan-1-one was replaced with 1-(5-amino-6-fluoro-2,3-dihydro-1H-isoindol-2-yl)-2,2,2-trifluoroethan-1-one. The title compound was isolated in 33% yield. MS (ESI) m/z: 429.0 [M+H]⁺

### Step 3: 2,2,2-trifluoro-1-(5-fluoro-6-((5-(trifluoromethyl)-4-(trimethylstannyl)pyrimidin-2-yl)amino)isoindolin-2-yl)ethan-1-one

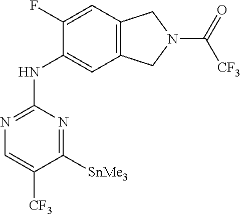

A mixture of 1-(5-((4-chloro-5-(trifluoromethyl)pyrimidin-2-yl)amino)-6-fluoroisoindolin-2-yl)-2,2,2-trifluoroethan-1-one (4.18 mmol), 1,4-bis(diphenylphosphino)butane (0.83 mmol), hexamethylditin (12.5 mmol) and palladium (II) acetate (0.83 mmol) in dioxane (36 mL) was stirred at 95° C. overnight. The reaction mixture was concentrated under reduced pressure and the crude product was purified by silica gel chromatography (10% triethylamine in hexanes) to afford the title compound in 75% yield. MS (ESI) m/z: 558.9 [M+H]⁺.

### Step 4: 2,2,2-trifluoro-1-(5-fluoro-6-((4-(4-(methylsulfonyl)thiophen-2-yl)-5-(trifluoromethyl)pyrimidin-2-yl)amino)isoindolin-2-yl)ethan-1-one

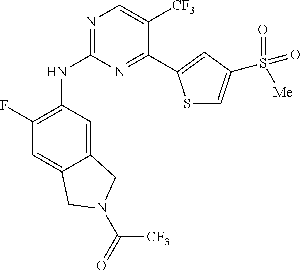 [SMILES: CS(=O)(=O)c1csc(-c2nc(Nc3cc4c(cc3F)CN(C(=O)C(F)(F)F)C4)ncc2C(F)(F)F)c1]

The title compound was prepared analogously to Example 4, step 2 where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 2-bromo-6,7-dihydrothieno[3,2-C]pyridin-4(5H)-one were replaced with 2,2,2-trifluoro-1-(5-fluoro-6-((5-(trifluoromethyl)-4-(trimethylstannyl)pyrimidin-2-yl)amino)isoindolin-2-yl)ethan-1-one and 2-bromo-4-methanesulfonylthiophene. The title compound was isolated in 78% yield. MS (ESI) m/z: 555.1 [M+H]⁺.

### Step 5: 6-fluoro-N-[4-(4-methanesulfonylthiophen-2-yl)-5-(trifluoromethyl)pyrimidin-2-yl]-2,3-dihydro-1H-isoindol-5-amine

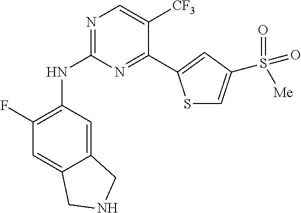 [SMILES: CS(=O)(=O)c1csc(-c2nc(Nc3cc4c(cc3F)CNC4)ncc2C(F)(F)F)c1]

The title compound was prepared analogously to Example 1, step 7 where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 2,2,2-trifluoro-1-(5-fluoro-6-((4-(4-(methylsulfonyl)thiophen-2-yl)-5-(trifluoromethyl)pyrimidin-2-yl)amino)isoindolin-2-yl)ethan-1-one. The title compound was isolated in 86% yield. MS (ESI) m/z: 459.4 [M+H]⁺. 1H-NMR (400 MHz, DMSO-d₆) δ 8.84 (s, 1H), 8.67 (d, J=1.4 Hz, 1H), 8.23 (s, 1H), 7.90 (s, 1H), 7.48 (d, J=7.2 Hz, 1H), 7.26 (d, J=10.2 Hz, 1H), 4.17 (d, J=7.4 Hz, 4H), 3.30 (s, 3H), 2.55 (s, 1H).

### Step 6: 6-fluoro-N-[4-(4-methanesulfonylthiophen-2-yl)-5-(trifluoromethyl)pyrimidin-2-yl]-2-methyl-2,3-dihydro-1H-isoindol-5-amine

The title compound was prepared analogously to Example 13, step 1 where methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 6-fluoro-N-[4-(4-methanesulfonylthiophen-2-yl)-5-(trifluoromethyl)pyrimidin-2-yl]-2,3-dihydro-1H-isoindol-5-amine. The title compound was isolated in 47% yield. MS (ESI) m/z: 473.3 [M+H]⁺. 1H-NMR (400 MHz, DMSO-d₆) δ 10.05 (s, 1H), 8.83 (s, 1H), 8.66 (d, J=1.3 Hz, 1H), 8.15 (s, OH), 7.89 (s, 1H), 7.43 (d, J=7.1 Hz, 1H), 7.19 (d, J=10.2 Hz, 1H), 3.82 (d, J=7.2 Hz, 4H), 3.29 (s, 3H).

### Example 82: -[2-[(7-chloro-1,2,3,4-tetrahydroisoquinolin-6-yl)amino]-5-(trifluoromethyl)pyrimidin-4-yl]-1,1-dioxo-3,4-dihydro-2H-thieno[2,3-f][1,4]thiazepin-5-one

### Step 1: 8-bromo-1,1-dioxo-3,4-dihydro-2H-thieno[2,3-f][1,4]thiazepin-5-one

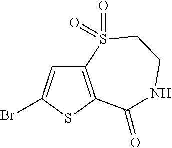 [SMILES: O=C1NCCS(=O)(=O)c2cc(Br)sc21]

A solution of 1,1-dioxo-3,4-dihydro-2H-thieno[2,3-f][1,4]thiazepin-5-one (13.8 mmol), N-bromosuccinimide (24.8 mmol) in acetic acid (30 mL) was treated with sulfuric acid (56.3 mmol) and the mixture was stirred at 60° C. for 12 hours. The pH was made neutral by the addition of sodium bicarbonate and the mixture was diluted with ethyl acetate, filtered, and concentrated. The residue was purified by preparative HPLC. The title compound was isolated in 10% yield. MS (ESI) m/z: 295.9 [M+H]⁺. ¹H NMR (400 MHz, DMSO-d₆) δ 8.86-8.80 (m, 1H), 7.69 (s, 1H), 3.93-3.86 (m, 2H), 3.69-3.64 (m, 2H).

### Step 2: 7-[2-[[7-chloro-2-(2,2,2-trifluoroacetyl)-3,4-dihydro-1H-isoquinolin-6-yl]amino]-5-(trifluoromethyl)pyrimidin-4-yl]-1,1-dioxo-3,4-dihydro-2H-thieno[2,3-f][1,4]thiazepin-5-one

The title compound was prepared analogously to Example 4, step 2 where 2-bromo-6,7-dihydrothieno[3,2-C]pyridin-4(5H)-one was replaced with 8-bromo-1,1-dioxo-3,4-dihydro-2H-thieno[2,3-f][1,4]thiazepin-5-one. The title compound was isolated in 24% yield. MS (ESI) m/z: 639.9 [M+H]⁺

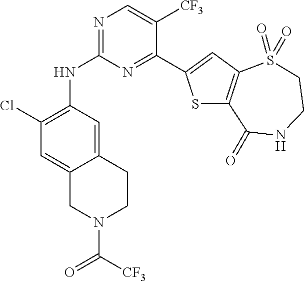 [SMILES: O=C1NCCS(=O)(=O)c2cc(-c3nc(Nc4cc5c(cc4Cl)CN(C(=O)C(F)(F)F)CC5)ncc3C(F)(F)F)sc21]

### Step 3: 7-[2-[(7-chloro-1,2,3,4-tetrahydroisoquinolin-6-yl)amino]-5-(trifluoromethyl)pyrimidin-4-yl]-1,1-dioxo-3,4-dihydro-2H-thieno[2,3-f][1,4]thiazepin-5-one

The title compound was prepared analogously to Example 59, step 6, where 5-(2-((7-chloro-2-(2,2,2-trifluoroacetyl)-1,2,3,4-tetrahydroisoquinolin-6-yl)amino)-5-(trifluoromethyl)pyrimidin-4-yl)-3-(methylsulfonyl)thiophene-2-carboxamide was replaced with 7-[2-[[7-chloro-2-(2,2,2-trifluoroacetyl)-3,4-dihydro-1H-isoquinolin-6-yl]amino]-5-(trifluoromethyl)pyrimidin-4-yl]-1,1-dioxo-3,4-dihydro-2H-thieno[2,3-f][1,4]thiazepin-5-one. The title compound was isolated in 62% yield. MS (ESI) m/z: 544.0 [M+H]⁺. ¹H NMR (400 MHz, DMSO-d₆) δ 10.16-9.72 (m, 1H), 8.90 (s, 1H), 8.83 (s, 1H), 7.85 (s, 1H), 7.29 (s, 1H), 7.21 (s, 1H), 4.42 (s, 1H), 3.91-3.87 (m, 2H), 3.83 (s, 2H), 3.67 (s, 2H), 2.92 (t, J=5.4 Hz, 2H), 2.67 (s, 3H).

### Example 83: 7-Chloro-N-[4-(4-methylsulfonyl-5-morpholino-2-thienyl)-5-(trifluoromethyl)pyrimidin-2-yl]-1,2,3,4-tetrahydroisoquinolin-6-amine

### Step 1: 4-(5-bromo-3-methylsulfonyl-2-thienyl)morpholine

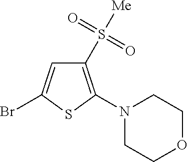 [SMILES: CS(=O)(=O)c1cc(Br)sc1N1CCOCC1]

A solution of 2,5-dibromo-3-methylsulfonyl-thiophene (1.72 mmol) in 1-methyl-2-pyrrolidinone (2.5 mL) was treated with morpholine (13.7 mmol) and N,N-diisopropylethylamine (13.75 mmol). The mixture was stirred at 135° C. for 1 hour. The reaction mixture was diluted with water, extracted with ethyl acetate three times and the combined organic layers were dried over anhydrous sodium sulfate, filtered, and concentrated under reduced pressure to afford a residue that was purified by preparative TLC (33% ethyl acetate in hexanes). The title compound was isolated in 45% yield. MS (ESI) m/z: 326.0[M+H]⁺.

### Step 2: 1-[7-chloro-6-[[4-(4-methylsulfonyl-5-morpholino-2-thienyl)-5-(trifluoromethyl)pyrimidin-2-yl]amino]-3,4-dihydro-1H-isoquinolin-2-yl]-2,2,2-trifluoro-ethanone

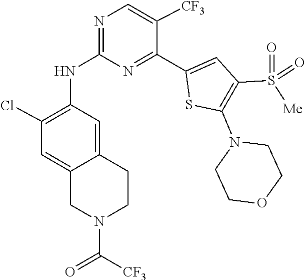 [SMILES: CS(=O)(=O)c1cc(-c2nc(Nc3cc4c(cc3Cl)CN(C(=O)C(F)(F)F)CC4)ncc2C(F)(F)F)sc1N1CCOCC1]

The title compound was prepared analogously to Example 4, step 2 where 2-bromo-6,7-dihydrothieno[3,2-C]pyridin-4(5H)-one were replaced with 4-(5-bromo-3-methylsulfonyl-2-thienyl)morpholine. The title compound was isolated in 63% yield. MS (ESI) m/z: 670.2 [M+H]⁺.

### Step 3: 7-Chloro-N-[4-(4-methylsulfonyl-5-morpholino-2-thienyl)-5-(trifluoromethyl)pyrimidin-2-yl]-1,2,3,4-tetrahydroisoquinolin-6-amine

The title compound was prepared analogously to Example 59, step 6 where 5-(2-((7-chloro-2-(2,2,2-trifluoroacetyl)-1,2,3,4-tetrahydroisoquinolin-6-yl)amino)-5-(trifluoromethyl)pyrimidin-4-yl)-3-(methylsulfonyl)thiophene-2-carboxamide was replaced with 1-[7-chloro-6-[[4-(4-methylsulfonyl-5-morpholino-2-thienyl)-5-(trifluoromethyl)pyrimidin-2-yl]amino]-3,4-dihydro-1H-isoquinolin-2-yl]-2,2,2-trifluoro-ethanone. The title compound was isolated in 39% yield. MS (ESI) m/z: 574.1 [M+H]⁺. ¹H NMR (400 MHz, DMSO-d₆) δ 9.74 (s, 1H), 8.71 (s, 1H), 8.26 (s, 1H), 7.82 (s, 1H), 7.39 (s, 1H), 7.24 (s, 1H), 3.90 (s, 2H), 3.81-3.76 (m, 4H), 3.36-3.33 (m, 4H), 3.29 (s, 3H), 3.00 (t, J=5.6 Hz, 2H), 2.76-2.70 (m, 2H).

### Example 85: 7-chloro-N-[4-(1,1-dioxo-3,4-dihydro-2H-thieno[2,3-b][1,4,5]oxathiazepin-7-yl)-5-(trifluoromethyl)pyrimidin-2-yl]-1,2,3,4-tetrahydroisoquinolin-6-amine

### Step 1: 2,5-dibromo-N-(2-hydroxyethyl)thiophene-3-sulfonamide

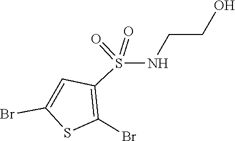 [SMILES: O=S(=O)(NCCO)c1cc(Br)sc1Br]

A 0° C. solution of 2,5-dibromothiophene-3-sulfonyl chloride (5.29 mmol) and triethylamine (15.9 mmol) in dichloromethane (20 mL) was treated with 2-aminoethanol (4.76 mmol). The mixture was stirred at 20° C. for 1 hour. The volatiles were removed under reduced pressure and the crude material purified by silica column chromatography (60% ethyl acetate in petroleum ether) to afford the title compound in 93% yield.

### Step 2: 7-bromo-3,4-dihydro-2H-thieno[2,3-b][1,4,5]oxathiazepine 1,1-dioxide

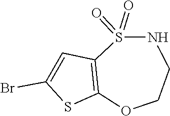 [SMILES: O=S1(=O)NCCOc2sc(Br)cc21]

A mixture of 2,5-dibromo-N-(2-hydroxyethyl)thiophene-3-sulfonamide (0.82 mmol), ethane-1,2-diol (1.5 mL), cuprous iodide (0.16 mmol) and cesium carbonate (2.47 mmol) in DMF (3 mL) was stirred at 120° C. for 12 hours. The reaction mixture was diluted with water, extracted with ethyl acetate three times and the combined organic layers were dried over anhydrous sodium sulfate, filtered, and concentrated under reduced pressure to afford a residue that was purified by preparative TLC (33% ethyl acetate in hexanes). The title compound was isolated in 9% yield. ¹H NMR (400 MHz, CDCl₃) δ 6.71 (s, 1H), 5.59 (s, 1H), 4.35 (t, J=4.4 Hz, 2H), 3.66 (s, 2H).

### Step 3: 1-[7-chloro-6-[[4-(1,1-dioxo-3,4-dihydro-2H-thieno[2,3-b][1,4,5]oxathiazepin-7-yl)-5-(trifluoromethyl)pyrimidin-2-yl]amino]-3,4-dihydro-1H-isoquinolin-2-yl]-2,2,2-trifluoro-ethanone

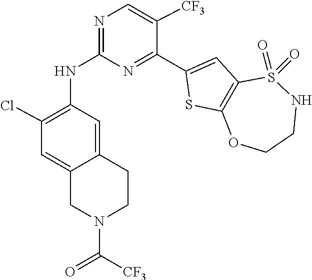 [SMILES: O=C(N1CCc2cc(Nc3ncc(C(F)(F)F)c(-c4cc5c(s4)OCCNS5(=O)=O)n3)c(Cl)cc2C1)C(F)(F)F]

The title compound was prepared analogously to Example 4, step 2 where 2-bromo-6,7-dihydrothieno[3,2-C]pyridin-4(5H)-one were replaced with 7-bromo-3,4-dihydro-2H-thieno[2,3-b][1,4,5]oxathiazepine 1,1-dioxide. The title compound was isolated. MS (ESI) m/z: 627.9 [M+H]⁺.

### Step 4: 7-chloro-N-[4-(1,1-dioxo-3,4-dihydro-2H-thieno[2,3-b][1,4,5]oxathiazepin-7-yl)-5-(trifluoromethyl)pyrimidin-2-yl]-1,2,3,4-tetrahydroisoquinolin-6-amine

The title compound was prepared analogously to Example 59, step 6 where 5-(2-((7-chloro-2-(2,2,2-trifluoroacetyl)-1,2,3,4-tetrahydroisoquinolin-6-yl)amino)-5-(trifluoromethyl)pyrimidin-4-yl)-3-(methylsulfonyl)thiophene-2-carboxamide was replaced with 1-[7-chloro-6-[[4-(1,1-dioxo-3,4-dihydro-2H-thieno[2,3-b][1,4,5]oxathiazepin-7-yl)-5-(trifluoromethyl)pyrimidin-2-yl]amino]-3,4-dihydro-1H-isoquinolin-2-yl]-2,2,2-trifluoro-ethanone. The title compound was isolated in 42% yield. MS (ESI) m/z: 532.1 [M+H]⁺. ¹H NMR (400 MHz, CDCl₃) δ 8.69 (s, 1H), 8.16 (s, 1H), 7.68 (s, 1H), 7.36 (s, 1H), 7.01 (s, 1H), 4.36-4.35 (m, 2H), 4.03 (s, 2H), 3.67 (s, 2H), 3.33 (s, 2H), 3.05 (s, 2H).

### Example 86: 7-(2-((7-chloro-2-methyl-1,2,3,4-tetrahydroisoquinolin-6-yl)amino)-5 (trifluoromethyl)pyrimidin-4-yl)-3,4-dihydrothieno[2,3-f][1,4]thiazepin-5(2H)-one 1,1-dioxide

The title compound was prepared analogously to Example 13, step 1 where methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 7-chloro-N-[4-(1,1-dioxo-3,4-dihydro-2H-thieno[2,3-b][1,4,5]oxathiazepin-7-yl)-5-(trifluoromethyl)pyrimidin-2-yl]-1,2,3,4-tetrahydroisoquinolin-6-amine. The title compound was isolated in 40% yield. MS (ESI) m/z: 558.1 [M+H]⁺. ¹H NMR (400 MHz, DMSO-d₆) δ 10.2220 (s, 1H), 10.08-10.00 (m, 1H), 8.91-8.84 (m, 2H), 7.89-7.86 (m, 1H), 7.58-7.53 (m, 1H), 7.51-7.49 (m, 1H), 4.60-4.48 (m, 1H), 4.37-4.28 (m, 1H), 3.93-3.89 (m, 2H), 3.71-3.64 (m, 3H), 3.51-3.46 (m, 1H), 3.14-3.07 (m, 2H), 2.97-2.94 (m, 3H).

### Example 88: 6-cyclopropyl-N-[4-(4-methanesulfonylthiophen-2-yl)-5-(trifluoromethyl)pyrimidin-2-yl]-2-methyl-2,3-dihydro-1H-isoindol-5-amine

### Step 1: 1-(5-amino-6-cyclopropyl-2,3-dihydro-1H-isoindol-2-yl)-2,2,2-trifluoroethan-1-one

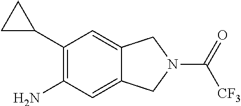 [SMILES: Nc1cc2c(cc1C1CC1)CN(C(=O)C(F)(F)F)C2]

The title compound was prepared analogously to Example 72, steps 1-4 where ethylboronic acid was replaced with cyclopropylboronic acid in step 2. The title compound was isolated. MS (ESI) m/z: 271.2 [M+H]⁺.

### Step 2: 1-(5-{[4-chloro-5-(trifluoromethyl)pyrimidin-2-yl]amino}-6-cyclopropyl-2,3-dihydro-1H-isoindol-2-yl)-2,2,2-trifluoroethan-1-one

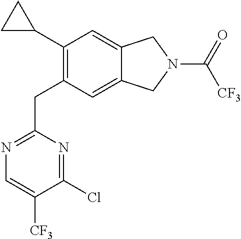 [SMILES: O=C(N1Cc2cc(Cc3ncc(C(F)(F)F)c(Cl)n3)c(C3CC3)cc2C1)C(F)(F)F]

The title compound was prepared analogously to Example 1, step 5 where 1-(6-amino-7-chloro-1,2,3,4-tetrahydroisoquinolin-2-yl)-2,2,2-trifluoroethan-1-one was replaced with 1-(5-amino-6-cyclopropyl-2,3-dihydro-1H-isoindol-2-yl)-2,2,2-trifluoroethan-1-one. The title compound was isolated in 24% yield. MS (ESI) m/z: 451.2 [M+H]⁺.

### Step 3: 1-(5-cyclopropyl-6-{[5-(trifluoromethyl)-4-(trimethylstannyl)pyrimidin-2-yl]amino}-2,3-dihydro-1H-isoindol-2-yl)-2,2,2-trifluoroethan-1-one

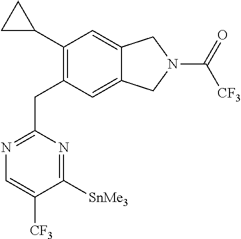

The title compound was prepared analogously to Example 80, step 3 where 1-(5-{[4-chloro-5-(trifluoromethyl)pyrimidin-2-yl]amino}-6-fluoro-2,3-dihydro-1H-isoindol-2-yl)-2,2,2-trifluoroethan-1-one was replaced with 1-(5-{[4-chloro-5-(trifluoromethyl)pyrimidin-2-yl]amino}-6-cyclopropyl-2,3-dihydro-1H-isoindol-2-yl)-2,2,2-trifluoroethan-1-one. The title compound was isolated in 10% yield. MS (ESI) m/z: 580.8 [M+H]⁺.

### Step 4: 1-(5-cyclopropyl-6-{[4-(4-methanesulfonylthiophen-2-yl)-5-(trifluoromethyl)pyrimidin-2-yl]amino}-2,3-dihydro-1H-isoindol-2-yl)-2,2,2-trifluoroethan-1-one

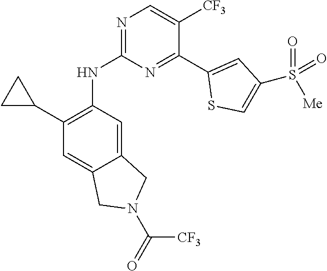 [SMILES: CS(=O)(=O)c1csc(-c2nc(Nc3cc4c(cc3C3CC3)CN(C(=O)C(F)(F)F)C4)ncc2C(F)(F)F)c1]

The title compound was prepared analogously to Example 4, step 2 where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 2-bromo-6,7-dihydrothieno[3,2-C]pyridin-4(5H)-one were replaced with 1-(5-cyclopropyl-6-{[5-(trifluoromethyl)-4-(trimethylstannyl)pyrimidin-2-yl]amino}-2,3-dihydro-1H-isoindol-2-yl)-2,2,2-trifluoroethan-1-one and 2-bromo-4-methanesulfonylthiophene. The title compound was isolated in 98% yield. MS (ESI) m/z: 577.0 [M+H]⁺.

### Step 5: 6-cyclopropyl-N-[4-(4-methanesulfonylthiophen-2-yl)-5-(trifluoromethyl)pyrimidin-2-yl]-2,3-dihydro-1H-isoindol-5-amine

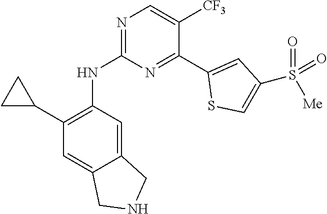 [SMILES: CS(=O)(=O)c1csc(-c2nc(Nc3cc4c(cc3C3CC3)CNC4)ncc2C(F)(F)F)c1]

The title compound was prepared analogously to Example 1, step 7 where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 1-(5-cyclopropyl-6-{[4-(4-methanesulfonylthiophen-2-yl)-5-(trifluoromethyl)pyrimidin-2-yl]amino}-2,3-dihydro-1H-isoindol-2-yl)-2,2,2-trifluoroethan-1-one. The title compound was isolated in 99% yield. MS (ESI) m/z: 481.4 [M+H]⁺.

### Step 6: 6-cyclopropyl-N-[4-(4-methanesulfonylthiophen-2-yl)-5-(trifluoromethyl)pyrimidin-2-yl]-2-methyl-2,3-dihydro-1H-isoindol-5-amine

The title compound was prepared analogously to Example 13, step 1 where methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 6-cyclopropyl-N-[4-(4-methanesulfonylthiophen-2-yl)-5-(trifluoromethyl)pyrimidin-2-yl]-2,3-dihydro-1H-isoindol-5-amine. The title compound was isolated in 25% yield. MS (ESI) m/z: 495.1 [M+H]⁺. 1H-NMR (400 MHz, DMSO-d₆) δ 0.58 (m, 2H), 0.82 (m, 2H), 1.96 (m, 1H), 2.53 (s, 3H), 3.28 (s, 3H), 3.86 (s, 4H), 6.88 (s, 1H), 7.26 (s, 1H), 7.87 (s, 1H), 8.15 (s, 1H), 8.64 (s, 1H), 8.77 (s, 1H), 9.84 (s, 1H).

### Example 95: 7-chloro-2-methyl-N-(4-(4-(methylsulfonyl)thiophen-2-yl)-5-(trifluoromethyl)pyrimidin-2-yl)-1,2,3,4-tetrahydroisoquinolin-6-amine

The title compound was prepared analogously to Example 13, step 1 where methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 7-chloro-N-(4-(4-(methylsulfonyl)thiophen-2-yl)-5-(trifluoromethyl)pyrimidin-2-yl)-1,2,3,4-tetrahydroisoquinolin-6-amine. The title compound was isolated in 15% yield. MS (ESI) m/z: 503.1 [M+H]⁺. 1H-NMR ((400 MHz, DMSO-d₆) δ 9.97 (s, 1H), 8.83 (s, 1H), 8.66 (d, J=1.3 Hz, 1H), 8.26 (s, 1H), 7.89 (s, 1H), 7.35 (s, 1H), 7.28 (s, 1H), 3.29 (s, 3H), 2.83 (t, J=6.0 Hz, 2H), 2.60 (t, J=5.9 Hz, 2H), 2.35 (s, 3H).

### Example 101: N-[4-(4-methanesulfonylthiophen-2-yl)-5-(trifluoromethyl)pyrimidin-2-yl]-1,2,3,4-tetrahydroisoquinolin-7-amine

### Step 1: 1-(7-{[4-chloro-5-(trifluoromethyl)pyrimidin-2-yl]amino}-1,2,3,4-tetrahydroisoquinolin-2-yl)-2,2,2-trifluoroethan-1-one

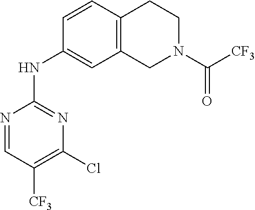 [SMILES: O=C(N1CCc2ccc(Nc3ncc(C(F)(F)F)c(Cl)n3)cc2C1)C(F)(F)F]

The title compound was prepared analogously to Example 1, step 5 where 1-(6-amino-7-chloro-1,2,3,4-tetrahydroisoquinolin-2-yl)-2,2,2-trifluoroethan-1-one was replaced with 1-(7-amino-1,2,3,4-tetrahydroisoquinolin-2-yl)-2,2,2-trifluoroethan-1-one. The title compound was isolated in 42% yield.

### Step 2: 2,2,2-trifluoro-1-(7-{[4-(4-methanesulfonylthiophen-2-yl)-5-(trifluoromethyl)pyrimidin-2-yl]amino}-1,2,3,4-tetrahydroisoquinolin-2-yl)ethan-1-one

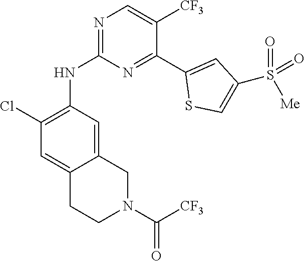 [SMILES: CS(=O)(=O)c1csc(-c2nc(Nc3cc4c(cc3Cl)CCN(C(=O)C(F)(F)F)C4)ncc2C(F)(F)F)c1]

The title compound was prepared analogously to Example 4, step 2 where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 2-bromo-6,7-dihydrothieno[3,2-C]pyridin-4(5H)-one were replaced with 1-(7-{[4-chloro-5-(trifluoromethyl)pyrimidin-2-yl]amino}-1,2,3,4-tetrahydroisoquinolin-2-yl)-2,2,2-trifluoroethan-1-one and (4-methanesulfonylthiophen-2-yl)trimethylstannane. The title compound was isolated in 83% yield. MS (ESI) m/z: 551.6 [M+H]⁺.

### Step 3: N-[4-(4-methanesulfonylthiophen-2-yl)-5-(trifluoromethyl)pyrimidin-2-yl]-1,2,3,4-tetrahydroisoquinolin-7-amine

The title compound was prepared analogously to Example 1, step 7 where methyl 5-(2-((7-chloro-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with 2,2,2-trifluoro-1-(7-{[4-(4-methanesulfonylthiophen-2-yl)-5-(trifluoromethyl)pyrimidin-2-yl]amino}-1,2,3,4-tetrahydroisoquinolin-2-yl)ethan-1-one. The title compound was isolated in 90% yield. MS (ESI) m/z: 455.1 [M+H]⁺. 1H-NMR (300 MHz, DMSO-d₆) δ 2.75 (d, J=6.2 Hz, 2H), 3.08 (m, 2H), 3.32 (s, 3H), 4.01 (s, 2H), 7.11 (d, J=8.4 Hz, 1H), 7.46 (dd, J=8.3, 2.2 Hz, 1H), 7.54 (s, 1H), 7.93 (s, 1H), 8.27 (s, 1H), 8.72 (d, J=1.3 Hz, 1H), 8.88 (s, 1H), 10.38 (s, 1H).

### Example 102: N-[4-(4-methanesulfonylthiophen-2-yl)-5-(trifluoromethyl)pyrimidin-2-yl]-2-methyl-1,2,3,4-tetrahydroisoquinolin-7-amine

The title compound was prepared analogously to Example 13, step 1 where methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with N-[4-(4-methanesulfonylthiophen-2-yl)-5-(trifluoromethyl)pyrimidin-2-yl]-1,2,3,4-tetrahydroisoquinolin-7-amine. The title compound was isolated in XX % yield. MS (ESI) m/z: 469.1 [M+H]⁺. 1H-NMR (400 MHz, DMSO-d₆) δ 2.39 (s, 3H), 2.66 (t, J=5.9 Hz, 2H), 2.81 (t, J=5.9 Hz, 2H), 3.32 (s, 3H), 3.57 (s, 2H), 7.10 (d, J=8.3 Hz, 1H), 7.45 (d, J=8.2 Hz, 1H), 7.51 (s, 1H), 7.93 (s, 1H), 8.15 (s, 2H), 8.73 (d, J=1.4 Hz, 1H), 8.87 (s, 1H), 10.36 (s, 1H).

### Example 116: 7-[2-[(7-chloro-2-methyl-3,4-dihydro-1H-isoquinolin-6-yl)amino]-5-(trifluoromethyl)pyrimidin-4-yl]-4-methyl-1,1-dioxo-2,3-dihydrothieno[2,3-f][1,4]thiazepin-5-one

### Step 1: 7-bromo-4-methyl-1,1-dioxo-2,3-dihydrothieno[2,3-f][1,4]thiazepin-5-one

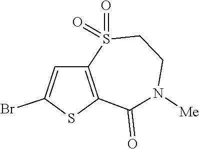 [SMILES: CN1CCS(=O)(=O)c2cc(Br)sc2C1=O]

A 0° C. solution of 7-bromo-1,1-dioxo-3,4-dihydro-2H-thieno[2,3-f][1,4]thiazepin-5-one (0.34 mmol) and DMF (3 mL) was treated with 60% sodium hydride (0.37 mmol). After 30 minutes, iodomethane (0.34 mmol) was added and the reaction was stirred at 20° C. for 12 hours. LCMS (EW33254-140-P1A) showed 43.03% of desired compound formed. The reaction mixture was diluted with water, extracted with ethyl acetate three times and the combined organic layers were dried over anhydrous sodium sulfate, filtered, and concentrated under reduced pressure to afford a residue that was purified by preparative TLC (100% ethyl acetate). The title compound was isolated in 81% yield. MS (ESI) m/z: 310.0 [M+H]⁺. ¹H NMR (400 MHz, DMSO-d₆) δ 7.95 (s, 1H), 3.94-3.93 (m, 2H), 3.92-3.90 (m, 2H), 3.06-3.06 (m, 3H).

### Step 2: 7-[2-[[7-chloro-2-(2,2,2-trifluoroacetyl)-3,4-dihydro-1H-isoquinolin-6-yl]amino]-5-(trifluoromethyl)pyrimidin-4-yl]-4-methyl-1,1-dioxo-2,3-dihydrothieno[2,3-f][1,4]thiazepin-5-one

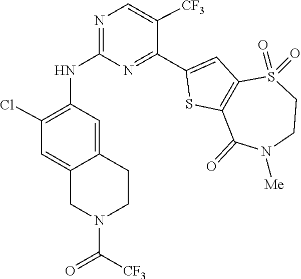 [SMILES: CN1CCS(=O)(=O)c2cc(-c3nc(Nc4cc5c(cc4Cl)CN(C(=O)C(F)(F)F)CC5)ncc3C(F)(F)F)sc2C1=O]

The title compound was prepared analogously to Example 4, step 2 where 2-bromo-6,7-dihydrothieno[3,2-C]pyridin-4(5H)-one was replaced with 7-bromo-4-methyl-1,1-dioxo-2,3-dihydrothieno[2,3-f][1,4]thiazepin-5-one. The title compound was isolated in 56% yield. MS (ESI) m/z: 654.0 [M+H]⁺.

### Step 3: 7-[2-[(7-chloro-1,2,3,4-tetrahydroisoquinolin-6-yl)amino]-5-(trifluoromethyl)pyrimidin-4-yl]-4-methyl-1,1-dioxo-2,3-dihydrothieno[2,3-f][1,4]thiazepin-5-one

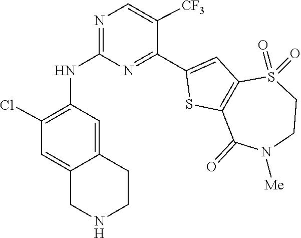 [SMILES: CN1CCS(=O)(=O)c2cc(-c3nc(Nc4cc5c(cc4Cl)CNCC5)ncc3C(F)(F)F)sc2C1=O]

The title compound was prepared analogously to Example 59, step 6 where 5-(2-((7-chloro-2-(2,2,2-trifluoroacetyl)-1,2,3,4-tetrahydroisoquinolin-6-yl)amino)-5-(trifluoromethyl)pyrimidin-4-yl)-3-(methylsulfonyl)thiophene-2-carboxamide was replaced with 7-[2-[[7-chloro-2-(2,2,2-trifluoroacetyl)-3,4-dihydro-1H-isoquinolin-6-yl]amino]-5-(trifluoromethyl)pyrimidin-4-yl]-4-methyl-1,1-dioxo-2,3-dihydrothieno[2,3-f][1,4]thiazepin-5-one. The title compound was isolated in 47% yield. MS (ESI) m/z: 558.1 [M+H]⁺.

### Step 4: 7-[2-[(7-chloro-2-methyl-3,4-dihydro-1H-isoquinolin-6-yl)amino]-5-(trifluoromethyl)pyrimidin-4-yl]-4-methyl-1,1-dioxo-2,3-dihydrothieno[2,3-f][1,4]thiazepin-5-one

A mixture of 7-[2-[(7-chloro-1,2,3,4-tetrahydroisoquinolin-6-yl)amino]-5-(trifluoromethyl)pyrimidin-4-yl]-4-methyl-1,1-dioxo-2,3-dihydrothieno[2,3-f][1,4]thiazepin-5-one (0.04 mmol) and paraformaldehyde (0.45 mmol) in methanol (1 mL) was stirred at 10° C. for 1 hour. Sodium cyanoborohydride (0.07 mmol) was added to the solution and the reaction was stirred at 10° C. for 12 hours. The reaction mixture was filtered and concentrated under reduced pressure to afford a crude product that was purified by preparative HPLC. The title compound was isolated in 31% yield. MS (ESI) m/z: 572.1 [M+H]⁺. ¹H NMR (400 MHz, DMSO-d₆) δ 10.23 (s, 1H), 10.05-9.94 (m, 1H), 8.90 (s, 1H), 7.84 (s, 1H), 7.54 (s, 1H), 7.50 (s, 1H), 4.59-4.45 (m, 1H), 4.37-4.27 (m, 1H), 4.01-3.95 (m, 2H), 3.92-3.86 (m, 2H), 3.77-3.64 (m, 1H), 3.18-3.03 (m, 6H), 2.96 (s, 3H).

Characterization Data for examples 40-120 are provided below in Table 2.

TABLE 2

| | m/z | |
|---|---|---|
| Example # | [M + H]⁺ | NMR |
| 40 | 494.32 | (400 MHz, DMSO-d₆) δ 9.80 (s, 1H), 8.77 (s, 1H), 8.67 (s, 1H), 7.56 (s, 1H), |
| | | 7.34 (s, 1H), 7.21 (s, 1H), 3.84 (s, 2H), 2.93 (t, J = 5.8 Hz, 2H), 2.70-2.63 |
| | | (m, 2H), 1.52 (s, 6H). |
| 41 | 552.15 | (400 MHz, DMSO-d₆) δ 9.77 (s, 1H), 8.74 (s, 1H), 8.29 (s, 1H), 7.83 (s, 1H), |
| | | 7.35 (s, 1H), 7.26 (s, 1H), 3.91 (s, 2H), 3.64 (s, 2H), 3.51 (s, 6H), 3.01 (s, |
| | | 3H), 2.73 (s, 4H), 2.07 (s, 2H). |
| 42 | 508.06 | (400 MHz, DMSO-d₆) δ 9.84 (s, 1H), 8.79 (s, 1H), 8.23 (s, 1H), 7.62 (s, 1H), |
| | | 7.39 (s, 1H), 7.27 (s, 1H), 3.93 (s, 2H), 3.03 (s, 2H), 2.90 (s, 3H), 2.75 (s, |
| | | 2H), 1.52 (s, 6H). |
| 44 | 558.03 | (400 MHz, DMSO-d₆) δ 9.89 (s, 1H), 8.79 (s, 1H), 7.90 (s, 1H), 7.27 (d, J = |
| | | 24.1 Hz, 2H), 4.12 (t, J = 6.3 Hz, 2H), 3.87 (s, 2H), 3.46 (s, 4H, overlapped |
| | | with H2O signal), 2.97 (s, 3H), 2.70 (s, 3H). |
| 45 | 468.06 | (400 MHz, DMSO-d₆) δ 2.64 (3H, s), 2.67 (2H, s), 2.76 (2H, s), 3.95 (2H, s), |
| | | 7.27 (1H, s), 7.37 (2H, s), 7.80 (1H, s), 8.74 (1H, s), 9.76 (1H, s). |
| 47 | 508 | (300 MHz, DMSO-d₆) δ 9.74 (s, 1H), 8.73 (s, 1H), 7.83 (s, 1H), 7.33 (s, 1H), |
| | | 7.22 (s, 1H), 3.86 (s, 2H), 3.36 (s, 2H), 3.04-2.99 (m, 2H), 3.02 (s, 3H), |
| | | 2.96 (t, J = 5.9 Hz, 2H), 2.69 (t, J = 6.2 Hz, 2H), 2.09 (p, J = 6.7 Hz, 2H). |
| 50 | 493.14 | (400 MHz, DMSO-d₆) δ 9.90 (s, 1H), 8.78 (s, 1H), 8.29 (s, 3H), 7.28 (s, 1H), |
| | | 7.21 (s, 1H), 3.86 (s, 2H), 2.96 (s, 2H), 2.65 (s, 2H), 2.33 (s, 3H). |
| 51 | 503 | (400 MHz, CDCl₃) δ = 8.76 (s, 1H), 8.27 (s, 1H), 8.15 (s, 1H), 8.04 (s, 1H), |
| | | 7.77 (s, 1H), 7.11 (s, 1H), 3.99 (s, 2H), 3.34-3.05 (m, 4H), 2.92-2.75 (m, |
| | | 2H), 1.36 (t, J = 7.4, 3H). |
| 52 | 533 | (400 MHz, CDCl₃) δ = 8.75 (s, 1H), 8.28 (s, 1H), 8.15 (s, 1H), 8.08 (s, 1H), |
| | | 7.76 (s, 1H), 7.11 (s, 1H), 3.99 (s, 2H), 3.82 (t, J = 5.8, 2H), 3.45 (t, J = 5.8, |
| | | 2H), 3.29 (s, 3H), 3.17 (t, J = 5.8, 2H), 2.86 (t, J = 5.8, 2H). |
| 54 | 588.1 | (400 MHz, CDCl₃) δ 8.76 (s, 1H), 8.28 (s, 1H), 8.15 (s, 1H), 8.04 (s, 1H), |
| | | 7.78 (s, 1H), 7.12 (s, 1H), 4.01 (s, 2H), 3.62-3.50 (m, 4H), 3.36 (t, J = 6.0, |
| | | 2H), 3.19 (t, J = 6.0, 2H), 2.92-2.79 (m, 4H), 2.45-2.32 (m, 4H). |
| 55 | 528.02 | (300 MHz, DMSO-d₆) δ 9.68 (s, 1H), 8.71 (d, J = 17.6 Hz, 1H), 7.89 (s, 1H), |
| | | 7.68-7.54 (m, 1H), 7.40 (s, 1H), 7.26 (s, 1H), 6.89 (s, 1H), 4.42 (d, J = 4.5 |
| | | Hz, 2H), 3.93 (s, 2H), 3.79 (s, 2H), 3.03 (s, 2H), 2.74 (d, J = 7.8 Hz, 2H). |
| 56 | 563.02 | (400 MHz, DMSO-d₆) δ 2.79 (t, J = 5.9 Hz, 2H), 3.09 (t, J = 6.0 Hz, 2H), |
| | | 3.23 (s, 3H), 3.34 (s, 3H), 3.78 (m, 2H), 3.99 (s, 2H), 4.49 (t, J = 4.2 Hz, 2H), |
| | | 7.30 (s, 1H), 7.40 (s, 1H), 7.71 (s, 1H), 8.20 (s, 1H), 8.73 (s, 1H), 9.83 (s, |
| | | 1H). |
| 57 | 577.25 | (400 MHz, DMSO-d₆) δ 2.34 (s, 3H), 2.59 (t, J = 5.9 Hz, 2H), 2.82 (t, J = 5.9 |
| | | Hz, 2H), 3.22 (s, 3H), 3.33 (s, 3H), 3.48 (s, 2H), 3.76 (m, 2H), 4.48 (t, J = 4.3 |
| | | Hz, 2H), 7.26 (s, 1H), 7.37 (s, 1H), 7.69 (s, 1H), 8.72 (s, 1H), 9.78 (s, 1H). |
| 58 | 602.1 | (400 MHz, DMSO-d₆) δ 10.06-9.96 (m, 1H), 8.84 (s, 1H), 8.21 (s, 1H), 7.84 |
| | | (s, 1H), 7.30 (s, 1H), 7.25 (s, 1H), 3.88 (s, 3H), 3.67-3.50 (m, 10H), 2.97 (t, |
| | | J = 5.6 Hz, 2H), 2.69 (s, 2H). |
| 61 | 616.2 | (400 MHz, DMSO-d₆) δ 10.02 (s, 1H), 8.84 (s, 1H), 7.84 (s, 1H), 7.34 (d, J = |
| | | 2.4 Hz, 1H), 7.29 (s, 1H), 3.69-3.52 (m, 8H), 3.30 (s, 5H), 2.82 (t, J = 5.6 |
| | | Hz, 2H), 2.65 (d, J = 4.4 Hz, 2H), 2.38 (s, 3H). |
| 62 | 588.1 | (400 MHz, CDCl₃) δ = 8.75 (m, 1H), 8.26 (s, 1H), 8.15-8.06 (m, 2H), 7.79 |
| | | (m, 1H), 7.27-7.15 (m, 1H), 4.07 (m, 4H), 3.77-3.75 (m, 4H), 3.26-3.22 |
| | | (m, 5H), 2.99 (m, 2H), 2.67 (m, 4H), 1.26 (m, 1H). |
| 65 | 583.9 | (400 MHz, DMSO-d₆) δ 9.62 (s, 1H), 8.68 (s, 1H), 7.68 (s, 1H), 7.21 (s, |
| | | 1H), 6.67 (s, 1H), 4.47 (s, 2H), 3.81-3.74 (m, 2H), 3.48 (s, 2H), 3.22 (s, 3H), |
| | | 2.80 (s, 2H), 2.62 (s, 2H), 2.36 (s, 2H), 1.93 (d, J = 14.5 Hz, 1H), 1.23 (s, |
| | | 1H), 0.86-0.77 (m, 2H), 0.59-0.51 (m, 2H). |
| 66 | 542.19 | (400 MHz, DMSO-d₆) δ 8.66 (s, 1H), 8.23 (s, 2H), 7.87 (s, 1H), 7.53 (s, |
| | | 1H), 7.42 (s, 1H), 7.25 (s, 1H), 6.92 (s, 1H), 4.44-4.40 (m, 2H), 3.77 (dd, J = |
| | | 5.7, 3.0 Hz, 2H), 3.50 (s, 2H), 3.31 (s, 3H), 2.82 (d, J = 6.3 Hz, 2H), 2.63 (d, |
| | | J = 5.8 Hz, 2H), 2.35 (s, 3H). |
| 67 | 602 | (400 MHz, CDCl₃) δ = 8.74 (d, J = 2.8 Hz, 1H), 8.28 (br d, J = 3.6 Hz, 1H), |
| | | 8.06 (s, 1H), 7.80 (br d, J = 4.0 Hz, 1H), 7.14 (d, J = 6.4 Hz, 1H), 4.10-4.06 |
| | | (m, 3H), 3.77-3.65 (m, 4H), 3.27-3.22 (m, 1H), 3.07-3.02 (m, 2H), 2.85 |
| | | (s, 1H), 2.83-2.68 (m, 1H), 2.67 (s, 4H), 2.66-2.54 (m, 2H). |
| 76 | 483.22 | (400 MHz, DMSO-d₆) δ 1.08 (t, J = 7.5 Hz, 3H), 2.50 (s, 3H), 2.58 (q, J = 7.6 |
| | | Hz, 2H), 3.28 (s, 3H), 3.82 (s, 2H), 3.83 (s, 2H), 7.16 (s, 1H), 7.20 (s, 1H), |
| | | 7.86 (s, 1H), 8.63 (s, 1H), 8.75 (s, 1H), 9.79 (s, 1H). |
| 78 | 618.9 | (400 MHz, CDCl₃) δ ppm 8.77 (s, 1 H), 8.10 (s, 1 H), 8.05 (s, 1 H), 7.73 (s, 1 |
| | | H), 7.13 (s, 1 H), 6.02 (t, J = 4.4 Hz, 1 H), 4.02 (s, 2 H), 3.86 (s, 2 H), 3.34- |
| | | 3.30 (m, 2 H), 3.25-3.18 (m, 4 H), 3.14 (s, 3 H), 2.92-2.85 (m, 2 H). |
| 79 | 502.9 | (400 MHz, CDCl₃) δ ppm 8.81 (s, 1H), 8.10 (d, J = 3.6 Hz, 1H), 7.81-7.71 |
| | | (m, 1H), 7.11 (s, 1H), 4.00 (s, 2H), 3.22 (s, 2H), 3.19-3.13 (m, 3H), 2.82 (s, |
| | | 2H), 2.47 (s, 2 H), 2.40 (s, 1H). |
| 81 | 459.09 | (400 MHz, DMSO-d₆) δ 8.84 (s, 1H), 8.67 (d, J = 1.4 Hz, 1H), 8.23 (s, 1H), |
| | | 7.90 (s, 1H), 7.48 (d, J = 7.2 Hz, 1H), 7.26 (d, J = 10.2 Hz, 1H), 4.17 (d, J = |
| | | 7.4 Hz, 4H), 3.30 (s, 3H), 2.55 (s, 1H). |
| 84 | 529.9 | (400 MHz, CDCl₃) δ 8.71 (s, 1 H), 8.21 (s, 1 H), 7.73 (s, 1 H), 7.53 (s, 1 H), |
| | | 7.08 (s, 1 H), 4.06 (s, 2 H), 3.71-3.68 (m, 2 H), 3.30-3.29 (m, 2 H), 3.18- |
| | | 3.15 (m, 2 H), 3.05-3.03 (m, 2 H), 1.88-1.85 (m, 2 H). |
| 87 | 587.2 | (400 MHz, DMSO-d₆) δ 8.74 (s, 1H), 7.98 (s, 1H), 7.85 (s, 1H), 7.26 (s, 1H), |
| | | 4.09-4.02 (m, 2H), 3.97-3.90 (m, 1H), 3.67-3.57 (m, 4H), 3.20 (s, 3H), |
| | | 3.06-3.00 (m, 2H), 2.85-2.80 (m, 2H), 2.50 (s, 3H), 2.10-2.03 (m, 2H), |
| | | 1.86-1.75 (m, 2H). |
| 89 | 461.26 | (400 MHz, DMSO-d₆) δ 8.76 (s, 1H), 8.57 (d, J = 1.3 Hz, 1H), 8.40 (s, 1H), |
| | | 8.31 (d, J = 1.4 Hz, 1H), 8.24 (s, 1H), 7.62 (s, 1H), 7.23 (s, 1H), 3.92 (s, 2H), |
| | | 3.05 (t, J = 5.9 Hz, 2H), 2.77 (t, J = 6.0 Hz, 2H), 2.14-2.05 (m, 1H), 1.10- |
| | | 1.00 (m, 2H), 0.79-0.69 (m, 2H). |
| 90 | 622 | (400 MHz, CDCl₃) δ ppm 9.96-9.92 (m, 2 H), 8.75 (s, 1 H), 8.33 (s, 1 H), |
| | | 8.01 (s, 1 H), 7.85 (s, 1 H), 7.25 (s, 1 H), 4.34 (s, 2 H), 3.85 (s, 4 H), 3.56 (d, |
| | | J = 5.2 Hz, 2 H), 3.34 (s, 4 H), 3.23 (s, 2 H), 3.18 (s, 3 H). |
| 91 | 473 | (400 MHz, DMSO-d₆) δ 10.22-10.16 (m, 1H), 9.12-8.99 (m, 2H), 8.89- |
| | | 8.83 (m, 1H), 8.73-8.65 (m, 1H), 7.95-7.88 (m, 1H), 7.55-7.48 (m, 1H), |
| | | 7.28-7.20 (m, 1H), 4.31-4.27 (m, 2H), 3.43-3.39 (m, 2H), 3.30-3.30 (m, |
| | | 3H), 3.02-2.97 (m, 2H). |
| 92 | 469.14 | (400 MHz, DMSO-d₆) δ 9.80 (s, 1H), 8.78 (s, 1H), 8.64 (s, 1H), 7.87 (s, 1H), |
| | | 7.25 (s, 1H), 7.14 (s, 1H), 3.86 (s, 4H), 3.28 (s, 3H), 2.19 (s, 3H). |
| 93 | 457 | (300 MHz, DMSO-d₆) δ 2.77 (s, 2H), 3.05 (s, 2H), 3.31 (s, 3H), 3.94 (s, 2H), |
| | | 7.26 (s, 1H), 7.44 (s, 1H), 8.24 (s, 1H), 8.41 (d, J = 1.4 Hz, 1H), 8.61 (s, 1H), |
| | | 8.63 (d, J = 1.4 Hz, 1H), 9.32 (s, 1H). |
| 94 | 435.02 | (300 MHz, DMSO-d₆) δ 2.77 (s, 2H), 3.05 (s, 2H), 3.31 (s, 3H), 3.94 (s, 2H), |
| | | 7.26 (s, 1H), 7.44 (s, 1H), 8.24 (s, 1H), 8.41 (d, J = 1.4 Hz, 1H), 8.61 (s, 1H), |
| | | 8.63 (d, J = 1.4 Hz, 1H), 9.32 (s, 1H). |
| 96 | 533 | (400 MHz, CDCl₃) δ 8.83 (s, 1H), 8.79 (s, 1H), 8.09 (s, 1H), 7.87 (s, 1H), |
| | | 7.46 (s, 1H), 4.23 (s, 2H), 3.86 (t, J = 6.8 Hz, 2H), 3.42 (t, J = 6.1 Hz, 2H), |
| | | 3.16 (s, 3H), 3.15 (s, 3H), 2.9 (m, 2H). |
| 97 | 573.1 | (400 MHz, methanol-d4) δ 8.77 (s, 1H), 8.01 (s, 1H), 7.99 (s, 1H), 7.42 (s, |
| | | 1H), 4.35 (s, 2H), 4.06 (dd, J = 4.0, 11.6 Hz, 2H), 3.95 (tt, J = 3.6, 11.6 Hz, |
| | | 1H), 3.64-3.57 (m, 2H), 3.53 (t, J = 6.4 Hz, 2H), 3.21 (s, 3H), 3.18 (t, J = |
| | | 6.4 Hz, 2H), 2.11-2.05 (m, 2H), 1.85-1.75 (m, 2H). |
| 98 | 487.1 | (400 MHz, DMSO-d₆) δ 10.24 (brs, 1H), 8.87 (s, 1H), 8.69 (s, 1H), 7.91 (s, |
| | | 1H), 7.59-7.47 (m, 1H), 7.28-7.17 (m, 1H), 4.48-4.32 (m, 1H), 4.26- |
| | | 3.96 (m, 1H), 3.31 (s, 3H), 3.16 (s, 3H), 3.11-3.04 (m, 2H), 2.96-2.94 (m, |
| | | 2H). |
| 99 | 488.92 | (400 MHz, DMSO-d₆) δ 2.74 (d, J = 5.9 Hz, 2H), 3.00 (t, J = 5.9 Hz, 2H), |
| | | 3.29 (s, 3H), 3.88 (s, 2H), 7.27 (s, 1H), 7.29 (s, 1H), 7.88 (s, 1H), 8.20 (s, |
| | | 1H), 8.66 (d, J = 1.3 Hz, 1H), 8.81 (s, 1H), 9.97 (s, 1H). |
| 100 | 502.99 | (400 MHz, DMSO-d₆) δ 2.39 (s, 3H), 2.67 (m, 2H), 2.85 (t, J = 5.9 Hz, 2H), |
| | | 3.29 (s, 3H), 3.54 (s, 2H), 7.29 (s, 1H), 7.33 (s, 1H), 7.89 (s, 1H), 8.13 (s, |
| | | 1H), 8.66 (d, J = 1.3 Hz, 1H), 8.82 (s, 1H), 9.97 (s, 1H). |
| 103 | 498.16 | (400 MHz, DMSO-d₆) δ 10.25 (s, 1H), 8.89-8.66 (m, 2H), 7.92 (s, 1H), |
| | | 7.58 (d, J = 8.4 Hz, 2H), 6.95 (d, J = 8.9 Hz, 2H), 3.32 (s, 3H), 3.11 (t, J = |
| | | 5.0 Hz, 4H), 2.46 (t, J = 5.0 Hz, 4H), 2.23 (s, 3H) |
| 104 | 621.1 | (400 MHz, CDCl₃) δ = 8.75 (s, 1H), 8.20 (s, 1H), 8.00 (s, 1H), 7.79 (s, 1H), |
| | | 7.14 (s, 1H), 4.04 (s, 2H), 3.25 (s, 2H), 3.20 (s, 2H), 3.17 (s, 3H), 2.94 (t, J = |
| | | 5.2 Hz, 2H), 2.51-2.48 (m, 3H), 1.36-1.29 (m, 4H) |
| 105 | 455 | (400 MHz, CDCl₃) δ 8.80 (s, 1 H), 8.54 (s, 1 H), 8.06 (s, 1 H), 7.77 (s, 1 H), |
| | | 7.60 (dd, J = 8.4, 2.0 Hz, 1 H), 7.21 (d, J = 8.4 Hz, 1 H), 4.33 (s, 2 H), 3.51 |
| | | (t, J = 6.4 Hz, 1 H), 3.21 (s, 3 H), 3.18-3.13 (m, 2 H). |
| 106 | 455 | (400 MHz, CDCl₃) δ 8.80 (s, 1 H), 8.53 (d, J = 0.8 Hz, 1 H), 8.05 (s, 1 H), |
| | | 7.80 (s, 1 H), 7.62 (dd, J = 4.4, 2.0 Hz, 1 H), 7.20 (d, J = 8.4 Hz, 1 H), 4.53- |
| | | 4.33 (m, 2 H), 3.79-3.45 (m, 2 H), 3.28-3.21 (m, 5 H), 3.07 (s, 3 H). |
| 107 | 529.1 | (400 MHz, methanol-d4) δ 8.78 (s, 1H), 8.50 (d, J = 1.2 Hz, 1H), 8.04 (s, 1H), |
| | | 7.85 (s, 1H), 7.35 (s, 1H), 4.20 (s, 2H), 3.44-3.34 (m, 2H), 3.19 (s, 3H), |
| | | 3.11 (t, J = 6.4 Hz, 2H), 2.56-2.36 (m, 1H), 0.92-0.75 (m, 4H). |
| 108 | 571.1 | (400 MHz, DMSO-d₆) δ 9.97 (s, 1H), 8.82 (s, 1H), 8.65 (s, 1H), 7.89 (s, 1H), |
| | | 7.36 (s, 1H), 7.29 (s, 1H), 3.83 (s, 2H), 3.37 (d, J = 10.0 Hz, 2H), 3.28 (s, |
| | | 3H), 2.96-2.92 (m, 2H), 2.84-2.80 (m, 2H). |
| 109 | 558.1 | (400 MHz, DMSO-d₆) δ 10.06 (s, 1H), 8.82 (s, 1H), 7.86 (s, 1H), 7.56 (s, |
| | | 1H), 7.47 (s, 1H), 4.56-4.49 (m, 1H), 4.34-4.27 (m, 3H), 3.71-3.57 (m, |
| | | 6H), 3.11-3.06 (m, 2H), 2.96 (s, 3H), 2.29 (s, 3H). |
| 110 | 473.03 | (300 MHz, DMSO-d₆) δ 10.08 (s, 1H), 8.83 (s, 1H), 8.67 (d, J = 1.3 Hz, 1H), |
| | | 7.90 (s, 1H), 7.31 (d, J = 7.9 Hz, 1H), 7.08 (d, J = 11.3 Hz, 1H), 3.96 (s, 2H), |
| | | 3.29 (s, 3H), 3.08 (t, J = 6.0 Hz, 2H), 2.79 (t, J = 5.9 Hz, 2H). |
| 111 | 487.39 | (300 MHz, DMSO-d₆) δ 10.03 (s, 1H), 8.83 (s, 1H), 8.68 (d, J = 1.3 Hz, 1H), |
| | | 8.19 (s, 1H), 7.89 (s, 1H), 7.28 (d, J = 8.0 Hz, 1H), 7.05 (d, J = 11.3 Hz, 1H), |
| | | 3.46 (s, 2H), 3.29 (s, 3H), 2.82 (t, J = 5.9 Hz, 2H), 2.59 (t, J = 5.9 Hz, 2H), |
| | | 2.34 (s, 3H) |
| 112 | 530.03 | (400 MHz, DMSO-d₆) δ 9.82 (s, 1H), 8.77 (s, 1H), 8.64 (s, 1H), 7.87 (s, 1H), |
| | | 7.32 (d, J = 8.8 Hz, 1H), 7.05 (d, J = 2.7 Hz, 1H), 6.94 (dd, J = 8.9, 2.8 Hz, |
| | | 1H), 3.28 (s, 3H), 3.18 (dd, J = 6.3, 3.8 Hz, 4H), 2.45 (t, J = 5.1 Hz, 4H), |
| | | 2.23 (s, 3H). |
| 113 | 533.11 | (300 MHz, DMSO-d₆) δ 10.00 (s, 1H), 8.82 (s, 1H), 8.65 (d, J = 1.3 Hz, 1H), |
| | | 7.89 (s, 1H), 7.44 (s, 2H), 3.98 (s, 4H), 3.29 (s, 3H), 2.07 (m, 1H), 0.45 (m, |
| | | 4H). |
| 114 | 499.26 | (300 MHz, DMSO-d₆) δ 10.21 (s, 1H), 8.86 (s, 1H), 8.68 (d, J = 1.3 Hz, 1H), |
| | | 7.91 (s, 1H), 7.60 (s, 1H), 7.36 (s, 1H), 4.62 (s, 4H), 3.30 (s, 3H), 0.73 (m, |
| | | 5H). |
| 115 | 509.1 | (400 MHz, methanol-d₄) δ 8.74-8.71 (m, 1H), 8.52-8.49 (m, 1H), 8.03 (s, |
| | | 1H), 7.61-7.57 (m, 1H), 6.87-6.82 (m, 1H), 3.77 (s, 2H), 3.31 (m, 3H), |
| | | 3.02 (br d, J = 6.0 Hz, 2H), 2.95-2.89 (m, 2H), 2.62-2.57 (m, 3H), 1.95- |
| | | 1.92 (m, 1H), 0.85-0.82 (m, 2H), 0.79-0.65 (m, 2H). |
| 117 | 570.1 | (400 MHz, methanol-d₄) δ 8.73 (s, 1H), 8.01 (s, 1H), 7.80 (s, 1H), 7.31 (s, |
| | | 1H), 4.23 (s, 2H), 4.07 (s, 2H), 3.57-3.50 (m, 2H), 3.42-3.34 (m, 2H), 3.24 |
| | | (d, J = 5.2 Hz, 2H), 3.05 (t, J = 6.0 Hz, 2H), 2.33-2.20 (m, 1H), 0.80-0.67 |
| | | (m, 4H). |
| 118 | 513.1 | 400 MHz, DMSO-d₆) δ 8.76 (s, 1H), 8.51 (s, 1H), 8.04 (s, 1H), 7.74 (m, 1H), |
| | | 6.97 (d, J = 12 Hz, 1H), 3.89 (m, 2H), 3.10 (s, 3H), 3.07 (m, 2H), 2.96 (m, |
| | | 2H), 2.02 (M, 1H), 0.64 (m, 4H) |
| 119 | 501.03 | (400 MHz, DMSO-d₆) δ 1.97 (m, 6H), 2.14 (m, 3H), 2.21 (s, 3H), 2.86 (d, |
| | | J = 10.9 Hz, 2H), 3.31 (s, 3H), 4.00 (m, 1H), 7.94 (m, 2H), 8.71 (m, 1H), |
| | | 8.80 (m, 1H), 9.74 (m, 1H). |
| 120 | 473.13 | (300 MHz, DMSO-d₆) δ 2.17 (m, 3H), 2.39 (s, 3H), 3.31 (s, 3H), 3.44 (m, |
| | | 2H), 3.77 (t, J = 7.8 Hz, 2H), 4.90 (d, J = 6.9 Hz, 1H), 7.91 (s, 1H), 8.05 (m, |
| | | 1H), 8.16 (s, 2H), 8.70 (s, 1H), 8.81 (m, 1H), 9.80 (m, 1H). |

### Example 121: N-(2-chloro-5-(1-methylazetidin-3-yl)phenyl)-4-(4-(methylsulfonyl)thiophen-2-yl)-5-(trifluoromethyl)pyrimidin-2-amine

### Step 1: tert-butyl 3-(2-tosylhydrazineylidene)azetidine-1-carboxylate

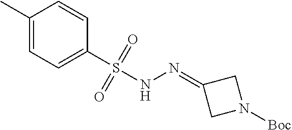 [SMILES: Cc1ccc(S(=O)(=O)NN=C2CN(C(=O)OC(C)(C)C)C2)cc1]

A mixture of p-toluenesulfonyl hydrazide (16.31 g, 87.6 mmol, 1.0 eq) and t-butyl 3-oxoazetidine-1-carboxylate (15.00 g, 87.6 mmol, 1.0 eq) in toluene (262 mL) was stirred at 110° C. for 2 h. The reaction mixture was filtered and the solid residue was dried overnight under vacuum to afford the title compound in 88% yield. MS (ESI) m/z: 240.0 [M+H-Boc]+.

### Step 2: tert-butyl 3-(3-amino-4-chlorophenyl)azetidine-1-carboxylate

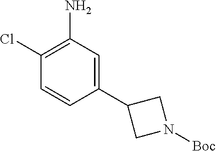 [SMILES: CC(C)(C)OC(=O)N1CC(c2ccc(Cl)c(N)c2)C1]

To a solution of tert-butyl 3-[(4-methylbenzenesulfonamido)imino]azetidine-1-carboxylate (6.00 g, 18 mmol, 1.00 eq) in anhydrous dioxane (126 mL), 3-amino-4-chlorophenylboronic acid (4.54 g, 26.516 mmol, 1.5 eq) and Cs₂CO₃ (8.64 g, 26.516 mmol) were added. The reaction mixture was refluxed at 110° C. for 30 h. The reaction mixture was and concentrated under reduced pressure to afford a residue that was purified by silica gel chromatography (0-30% ethyl acetate in hexanes). The title compound was isolated in 30% yield. MS (ESI) m/z: 277.0 [M+H-Boc]+.

### Step 3: tert-butyl 3-(4-chloro-3-((4-chloro-5-(trifluoromethyl)pyrimidin-2-yl)amino) phenyl)azetidine-1-carboxylate

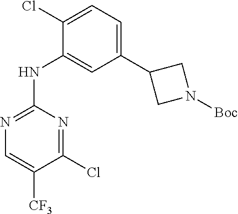 [SMILES: CC(C)(C)OC(=O)N1CC(c2ccc(Cl)c(Nc3ncc(C(F)(F)F)c(Cl)n3)c2)C1]

To a 0° C. solution of 2,4-dichloro-5-trifluoromethyl-pyrimidine (0.219 mmol) in dichloroethane/t-BuOH (1.2 mL/0.2 mL), a 0.7 M solution of ZnCl2 in THF (0.69 mL) was added. The reaction mixture was stirred at 0° C. for 1 hour and a solution of tert-butyl 3-(3-amino-4-chlorophenyl)azetidine-1-carboxylate (0.219 mmol) in dichloroethane/t-BuOH [0.31 mL/0.31 mL] was added. Over the resulting mixture, diisopropylethylamine (0.241 mmol) was added dropwise and the reaction was stirred overnight at room temperature. Water was added and the resulting solution was extracted with ethyl acetate three times. The combined organic layers were dried over anhydrous sodium sulfate, filtered, and concentrated under reduced pressure to afford a residue that was purified by silica gel chromatography (10% methanol in dichloromethane). The title compound was isolated in 21% yield. m/z (ESI, +ve)=408.8 [M+H]⁺.

### Step 4: tert-butyl 3-(4-chloro-3-((4-(4-(methylsulfonyl)thiophen-2-yl)-5-(trifluoromethyl)pyrimidin-2-yl)amino)phenyl)azetidine-1-carboxylate

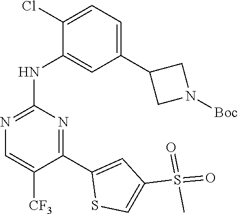 [SMILES: CC(C)(C)OC(=O)N1CC(c2ccc(Cl)c(Nc3ncc(C(F)(F)F)c(-c4cc(S(C)(=O)=O)cs4)n3)c2)C1]

A mixture of tert-butyl 3-(4-chloro-3-((4-chloro-5-(trifluoromethyl)pyrimidin-2-yl)amino)phenyl)azetidine-1-carboxylate (1.47 mmol), trimethyl(4-(methylsulfonyl)thiophen-2-yl)stannane (1.91 mmol) and tetrakis(triphenylphosphine)palladium (0) (0.073 mmol) in dioxane (7 mL) was stirred at 100° C. for 16 hours. Evaporation of volatiles under reduced pressure afforded a residue that was purified by silica gel chromatography (0-60% ethyl acetate in hexanes). The title compound was isolated in 69% yield. m/z (ESI, +ve)=489.0 (M+H-Boc)⁺.

### Step 5: N-(5-(azetidin-3-yl)-2-chlorophenyl)-4-(4-(methylsulfonyl)thiophen-2-yl)-5-(trifluoromethyl)pyrimidin-2-amine

A 4M solution of HCl in methanol (5 mL) was added over a solution of tert-butyl 3-(4-chloro-3-((4-(4-(methylsulfonyl)thiophen-2-yl)-5-(trifluoromethyl)pyrimidin-2-yl)amino)phenyl)azetidine-1-carboxylate (1 mmol) in methanol (6 mL). After 90 minutes the volatiles were removed under reduced pressure to afford the title compound which was used in the next step without further purification. m/z (ESI, +ve)=489.0 [M+H]⁺. ¹H NMR (300 MHz, DMSO-d₆) δ 8.85 (s, 1H), 8.65 (d, J=1.3 Hz, 1H), 8.32 (s, 1H), 7.89 (s, 1H), 7.63 (d, J=2.1 Hz, 1H), 7.55 (d, J=8.3 Hz, 1H), 7.32 (dd, J=8.3, 2.2 Hz, 1H), 4.04 (d, J=3.3 Hz, 3H), 3.86 (s, 2H), 3.29 (s, 3H).

### Example 122: N-(2-chloro-5-(1-methylazetidin-3-yl)phenyl)-4-(4-(methylsulfonyl)thiophen-2-yl)-5-(trifluoromethyl)pyrimidin-2-amine

The title compound was prepared analogously to Example 17, where 5-(2-((6-chloroisoindolin-5-yl)amino)-5-(trifluoromethyl)pyrimidin-4-yl)thiophene-3-carboxamide was replaced with N-(5-(azetidin-3-yl)-2-chlorophenyl)-4-(4-(methylsulfonyl)thiophen-2-yl)-5-(trifluoromethyl)pyrimidin-2-amine. The title compound was isolated in 46% yield. m/z (ESI, +ve)=503.0 [M+H]⁺. ¹H NMR (300 MHz, DMSO-d₆) δ 10.05 (s, 1H), 8.84 (s, 1H), 8.66 (d, J=1.3 Hz, 1H), 8.20 (s, 1H), 7.89 (s, 1H), 7.59 (d, J=2.1 Hz, 1H), 7.51 (d, J=8.3 Hz, 1H), 7.29 (dd, J=8.3, 2.2 Hz, 1H), 3.78-3.64 (m, 3H), 3.28 (s, 3H), 3.24 (d, J=6.4 Hz, 2H), 2.33 (s, 3H).

### Example 123: N-(1-(azetidin-3-yl)-3-methyl-1H-pyrazol-4-yl)-4-(4-(methylsulfonyl)thiophen-2-yl)-5-(trifluoromethyl)pyrimidin-2-amine

### Step 1: tert-butyl 3-(3-methyl-4-nitro-1H-pyrazol-1-yl)azetidine-1-carboxylate

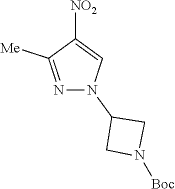 [SMILES: Cc1nn(C2CN(C(=O)OC(C)(C)C)C2)cc1[N+](=O)[O-]]

A solution of 3-methyl-4-nitro-1H-pyrazole (15.7 mmol) in THF (24 mL) was treated with 1-Boc-3-hydroxyazetidine (18.9 mmol) and triphenylphosphine (6.19 mmol). The mixture was cooled down to 0° C. and a 40% solution of diethylazodiacarboxylate in toluene (24 mmol) was added slowly. The reaction was stirred at room temperature overnight and concentrated under reduced pressure to afford a residue that was purified by silica gel chromatography (15-30% ethyl acetate in hexanes). The title compound was isolated in 45% yield. m/z (ESI, +ve)=227.0 (M+H-Boc)⁺.

### Step 2: tert-butyl 3-(4-amino-3-methyl-1H-pyrazol-1-yl)azetidine-1-carboxylate

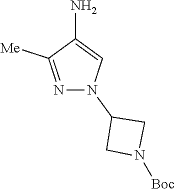 [SMILES: Cc1nn(C2CN(C(=O)OC(C)(C)C)C2)cc1N]

The title compound was prepared analogously to Example 19, step 3 where 1-(5-cyclopropyl-6-nitroisoindolin-2-yl)-2,2,2-trifluoroethan-1-one was replaced with tert-butyl 3-(3-methyl-4-nitro-1H-pyrazol-1-yl)azetidine-1-carboxylate. The title compound was isolated in 94% yield. ¹H NMR (300 MHz, DMSO-d₆) δ 1.41 (s, 9H), 2.04 (s, 3H), 3.70 (s, 2H), 4.03 (br.s, 2H), 4.18 (t, J=8.3 Hz, 2H), 4.92 (m, 1H), 7.06 (s, 1H).

### Step 3: tert-butyl 3-(4-((4-chloro-5-(trifluoromethyl)pyrimidin-2-yl)amino)-3-methyl-1H-pyrazol-1-yl)azetidine-1-carboxylate

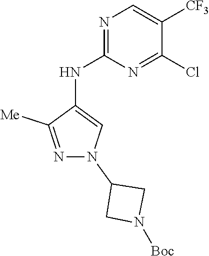 [SMILES: Cc1nn(C2CN(C(=O)OC(C)(C)C)C2)cc1Nc1ncc(C(F)(F)F)c(Cl)n1]

The title compound was prepared analogously to Example 121, step 3, where tert-butyl 3-(3-amino-4-chlorophenyl)azetidine-1-carboxylate was replaced with tert-butyl 3-(4-amino-3-methyl-1H-pyrazol-1-yl)azetidine-1-carboxylate. The title compound was isolated in 64% yield. 1H-NMR (300 MHz, DMSO-d₆) δ 1.40 (s, 9H), 2.16 (s, 3H), 4.10 (s, 2H), 4.25 (t, J=8.3 Hz, 2H), 5.13 (m, 1H), 7.97 (m, 1H), 8.71 (m, 1H), 10.07 (s, 1H).

### Step 4: tert-butyl 3-(3-methyl-4-((4-(4-(methylsulfonyl)thiophen-2-yl)-5-(trifluoromethyl)pyrimidin-2-yl)amino)-1H-pyrazol-1-yl)azetidine-1-carboxylate

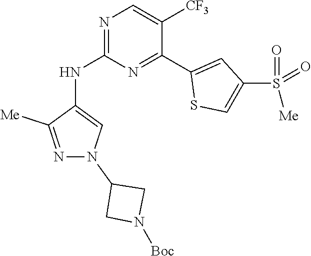 [SMILES: Cc1nn(C2CN(C(=O)OC(C)(C)C)C2)cc1Nc1ncc(C(F)(F)F)c(-c2cc(S(C)(=O)=O)cs2)n1]

The title compound was prepared analogously to Example 121, step 4 where tert-butyl 3-(4-chloro-3-((4-chloro-5-(trifluoromethyl)pyrimidin-2-yl)amino) phenyl)azetidine-1-carboxylate was replaced with tert-butyl 3-(4-((4-chloro-5-(trifluoromethyl)pyrimidin-2-yl)amino)-3-methyl-1H-pyrazol-1-yl)azetidine-1-carboxylate. The title compound was isolated in 68% yield. m/z (ESI, −ve)=557.1 (M−H)−.

### Step 5: N-(1-(azetidin-3-yl)-3-methyl-1H-pyrazol-4-yl)-4-(4-(methylsulfonyl)thiophen-2-yl)-5-(trifluoromethyl)pyrimidin-2-amine

The title compound was prepared analogously to Example 48, step 5 where tert-butyl 4-(3-cyclopropyl-4-((4-(4-oxo-4,5,6,7-tetrahydrothieno[3,2-c]pyridin-2-yl)-5-(trifluoromethyl)pyrimidin-2-yl)amino)-1H-pyrazol-1-yl)piperidine-1-carboxylate was replaced with tert-butyl 3-(3-methyl-4-((4-(4-(methylsulfonyl)thiophen-2-yl)-5-(trifluoromethyl)pyrimidin-2-yl)amino)-1H-pyrazol-1-yl)azetidine-1-carboxylate. The title compound was isolated in 98% yield. m/z (ESI, +ve)=459.0 [M+H]⁺. ¹H NMR (300 MHz, DMSO-d₆) δ 2.19 (m, 3H), 3.31 (s, 3H), 4.05 (m, 4H), 5.20 (m, 1H), 7.90 (s, 1H), 8.04 (m, 1H), 8.28 (s, 1H), 8.69 (s, 1H), 8.80 (m, 1H), 9.83 (m, 1H).

### Example 124: N-(3-methyl-1-(1-methylazetidin-3-yl)-1H-pyrazol-4-yl)-4-(4-(methylsulfonyl)thiophen-2-yl)-5-(trifluoromethyl)pyrimidin-2-amine

The title compound was prepared analogously to Example 17, where 5-(2-((6-chloroisoindolin-5-yl)amino)-5-(trifluoromethyl)pyrimidin-4-yl)thiophene-3-carboxamide was replaced with N-(1-(azetidin-3-yl)-3-methyl-1H-pyrazol-4-yl)-4-(4-(methylsulfonyl)thiophen-2-yl)-5-(trifluoromethyl)pyrimidin-2-amine. The title compound was isolated in 50% yield. m/z (ESI, +ve)=473.0 [M+H]⁺. ¹H NMR (300 MHz, DMSO-d₆) δ 2.16 (m, 3H), 2.34 (s, 3H), 3.31 (s, 3H), 3.36 (m, 2H), 3.70 (t, J=6.7 Hz, 2H), 4.86 (p, J=6.8 Hz, 1H), 8.02 (m, 2H), 8.77 (m, 2H), 9.79 (m, 1H).

### Example 125: 7-(2-((7-chloro-2-(2,2,2-trifluoroethyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

### Step 1: methyl 3-((2-((tert-butoxycarbonyl)amino)ethyl)sulfonyl)thiophene-2-carboxylate

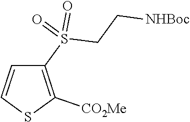 [SMILES: COC(=O)c1sccc1S(=O)(=O)CCNC(=O)OC(C)(C)C]

A mixture of methyl 3-chlorosulfonylthiophene-2-carboxylate (125 mmol), sodium sulfite (249 mmol), sodium bicarbonate (262 mmol) in ethanol (100 mL) and water (200 mL) was heated at 50° C. for 45 minutes. The mixture was concentrated under reduced pressure and the residue was taken up in DMF (300 mL) and treated with tert-butyl N-(2-bromoethyl)carbamate (249 mmol) and KI (374 mmol). After 12 hours, the organic volatiles were evaporated and the resulting aqueous mixture was poured into water and extracted with ethyl acetate three times. The combined organic layers were dried over anhydrous sodium sulfate, filtered, and concentrated under reduced pressure to afford a residue that was purified by preparative HPLC. The title compound was isolated in 90% yield. MS (ESI) m z: 250.1 [M+H]⁺. ¹H NMR (400 MHz, DMSO-d₆) δ 8.08-8.01 (m, 1H), 7.54-7.50 (m, 1H), 6.88-6.78 (m, 1H), 3.87 (s, 3H), 3.80-3.73 (m, 2H), 3.29-3.21 (m, 2H), 1.31 (s, 9H).

### Step 2: methyl 3-((2-aminoethyl)sulfonyl)thiophene-2-carboxylate

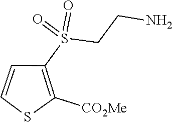 [SMILES: COC(=O)c1sccc1S(=O)(=O)CCN]

A 10° C. solution of methyl 3-((2-((tert-butoxycarbonyl)amino)ethyl)sulfonyl)thiophene-2-carboxylate (37 mmol) in dichloromethane (30 mL) was treated with TFA (405 mmol). Evaporation of volatiles afforded the title compound which was used in the next step without further purification. MS (ESI) m/z: 250.0[M+H]⁺.

### Step 3: 3,4-dihydrothieno[2,3-f][1,4]thiazepin-5(2H)-one 1,1-dioxide

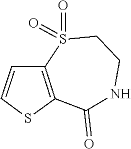 [SMILES: O=C1NCCS(=O)(=O)c2ccsc21]

A mixture of methyl 3-((2-aminoethyl)sulfonyl)thiophene-2-carboxylate (39 mmol) and potassium carbonate (154 mmol) in ethanol was refluxed for 12 hours. The reaction was cooled down to room temperature, filtered and concentrated under reduced pressure to afford the title compound that was used in the next step without further purification. ¹H NMR (400 MHz, DMSO-d₆) δ 8.82-8.63 (m, 1H), 8.05-8.01 (m, 1H), 7.53-7.49 (m, 1H), 3.87-3.81 (m, 2H), 3.66-3.59 (m, 2H).

### Step 4: 7-bromo-3,4-dihydrothieno[2,3-f][1,4]thiazepin-5(2H)-one 1,1-dioxide

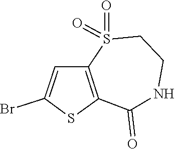 [SMILES: O=C1NCCS(=O)(=O)c2cc(Br)sc21]

A solution of 3,4-dihydrothieno[2,3-f][1,4]thiazepin-5(2H)-one 1,1-dioxide (28 mmol) in sulfuric acid (6 mL) and acetic acid (60 mL) was treated with N-bromosuccinimide (50 mmol) and the reaction was stirred at 60° C. for 12 hours. The mixture was cooled down to room temperature and the pH was neutralized by the addition of sodium bicarbonate. DMF added and the insoluble materials were filtered. The resulting solution was concentrated under reduced pressure to afford a residue that was purified by preparative HPLC. The title compound was isolated in 17% yield. MS (ESI) m/z: 295.9 [M+H]⁺. ¹H NMR (400 MHz, DMSO-d₆) δ 8.89-8.80 (m, 1H), 7.70 (s, 1H), 3.93-3.87 (m, 2H), 3.70-3.63 (m, 2H).

### Step 5: 7-(2-((7-chloro-2-(2,2,2-trifluoroacetyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

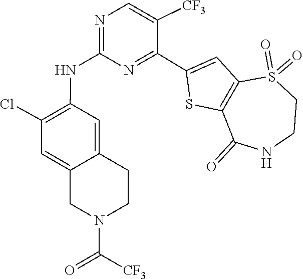 [SMILES: O=C1NCCS(=O)(=O)c2cc(-c3nc(Nc4cc5c(cc4Cl)CN(C(=O)C(F)(F)F)CC5)ncc3C(F)(F)F)sc21]

A mixture of 7-bromo-3,4-dihydrothieno[2,3-f][1,4]thiazepin-5(2H)-one 1,1-dioxide (0.68 mmol), 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one (0.68 mmol), tetrakis[triphenylphosphine]palladium(0) (0.07 mmol) and CuI (0.68 mmol) in dioxane (6 mL) was stirred at 120° C. for 12 hours. The reaction was concentrated and the residue was taken up in water and extracted with ethyl acetate three times. The combined organic layers were dried over anhydrous sodium sulfate, filtered, and concentrated under reduced pressure to afford a residue that was purified by silica gel chromatography (ethyl acetate). The title compound was isolated in 23% yield. MS (ESI) m/z: 639.9 [M+H]⁺.

### Step 6: 7-(2-((7-chloro-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

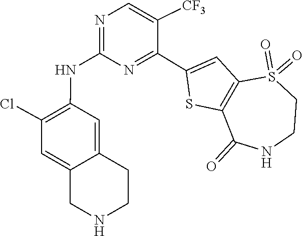 [SMILES: O=C1NCCS(=O)(=O)c2cc(-c3nc(Nc4cc5c(cc4Cl)CNCC5)ncc3C(F)(F)F)sc21]

A mixture of 7-(2-((7-chloro-2-(2,2,2-trifluoroacetyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide (0.16 mmol) and potassium carbonate (0.94 mmol) in ethanol (2 mL) and water was stirred at 50° C. for 12 hours. The mixture was concentrated and the residue was purified by preparative TLC (ethyl acetate) to afford the title compound in 55% yield. MS (ESI) m/z: 544.0 [M+H]⁺.

### Step 7: 7-(2-((7-chloro-2-(2,2,2-trifluoroethyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

Triethylamine (0.30 mmol) and 2,2,2-trifluoroethyl trifluoromethanesulfonate (0.14 mmol) were added over a solution of 7-(2-((7-chloro-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide in acetonitrile (1 mL). The reaction was stirred at 50° C. for 2 hours and concentrated under reduced pressure. The residue was taken up in water and extracted with ethyl acetate three times. The combined organic layers were dried over anhydrous sodium sulfate, filtered, and concentrated under reduced pressure to afford a residue that was purified by HPLC. The title compound was isolated in 18% yield. MS (ESI) m z: 626.0 [M+H]⁺. ¹H NMR (400 MHz, DMSO-d₆) δ 10.07 (s, 1H), 8.90-8.83 (m, 2H), 7.86 (s, 1H), 7.38 (br s, 1H), 7.30 (s, 1H), 3.91-3.87 (m, 2H), 3.86-3.83 (m, 2H), 3.68-3.65 (m, 2H), 3.41-3.34 (m, 2H), 2.97-2.91 (m, 2H), 2.86-2.80 (m, 2H).

### Example 126: 7-(2-((7-chloro-2-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

A solution of 7-(2-((7-chloro-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide (0.07 mmol) in THF (0.3 mL) and methanol (0.3 mL) was treated with acetic acid (0.007 mmol) and (1-ethoxycyclopropoxy)-trimethyl-silane (0.14 mmol). After 1 hour, sodium cyanoborohydride (0.10 mmol) was added and the reaction stirred at 60° C. for 12 hours. The reaction was quenched with water and concentrated. The crude residue was purified by preparative HPLC to afford the title compound in 22% yield. MS (ESI) m z: 584.0 [M+H]⁺. ¹H NMR (400 MHz, DMSO-d₆) δ 10.04 (s, 1H), 8.87-8.82 (m, 2H), 7.86 (s, 1H), 7.36-7.28 (m, 2H), 3.91-3.87 (m, 2H), 3.74-3.70 (m, 2H), 3.68-3.64 (m, 2H), 2.86-2.82 (m, 2H), 2.80-2.76 (m, 2H), 1.84-1.78 (m, 1H), 0.54-0.47 (m, 2H), 0.44-0.38 (m, 2H).

### Example 127: 8-chloro-N-(4-(4-(methylsulfonyl)thiophen-2-yl)-5-(trifluoromethyl)pyrimidin-2-yl)-2,3,4,5-tetrahydro-1H-benzo[c]azepin-7-amine

### Step 1: 1-(8-chloro-1,3,4,5-tetrahydro-2H-benzo[c]azepin-2-yl)-2,2,2-trifluoroethan-1-one

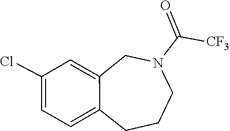 [SMILES: O=C(N1CCCc2ccc(Cl)cc2C1)C(F)(F)F]

A 0° C. solution of 8-chloro-2,3,4,5-tetrahydro-1H-benzo[c]azepine (56 mmol) in dichloromethane (200 mL) was treated with trifluoroacetic anhydride (67 mmol) and triethylamine (112 mmol). After 8 hours at room temperature, the reaction mixture was diluted with water, extracted with ethyl acetate three times and the combined organic layers were dried over anhydrous sodium sulfate, filtered, and concentrated under reduced pressure to afford the title compound in 96% yield. MS (ESI) m z: 278.0 [M+H]⁺.

### Step 2: 1-(8-chloro-7-nitro-1,3,4,5-tetrahydro-2H-benzo[c]azepin-2-yl)-2,2,2-trifluoroethan-1-one

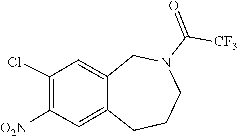 [SMILES: O=C(N1CCCc2cc([N+](=O)[O-])c(Cl)cc2C1)C(F)(F)F]

The title compound was prepared analogously to Example 1, step 3 where 1-(7-chloro-1,2,3,4-tetrahydroisoquinolin-2-yl)-2,2,2-trifluoroethan-1-one was replaced with 1-(8-chloro-1,3,4,5-tetrahydro-2H-benzo[c]azepin-2-yl)-2,2,2-trifluoroethan-1-one. The title compound was isolated in 97% yield. MS (ESI) m/z: 323.1 [M+H]⁺.

### Step 3: 1-(7-amino-8-chloro-1,3,4,5-tetrahydro-2H-benzo[c]azepin-2-yl)-2,2,2-trifluoroethan-1-one

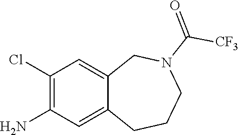 [SMILES: Nc1cc2c(cc1Cl)CN(C(=O)C(F)(F)F)CCC2]

The title compound was prepared analogously to Example 1, step 4 where 1-(7-chloro-6-nitro-1,2,3,4-tetrahydroisoquinolin-2-yl)-2,2,2-trifluoroethan-1-one was replaced with 1-(8-chloro-7-nitro-1,3,4,5-tetrahydro-2H-benzo[c]azepin-2-yl)-2,2,2-trifluoroethan-1-one. The title compound was isolated in 97% yield. MS (ESI) m/z: 293.0 [M+H]⁺.

### Step 4: 1-(8-chloro-7-((4-chloro-5-(trifluoromethyl)pyrimidin-2-yl)amino)-1,3,4,5-tetrahydro-2H-benzo[c]azepin-2-yl)-2,2,2-trifluoroethan-1-one

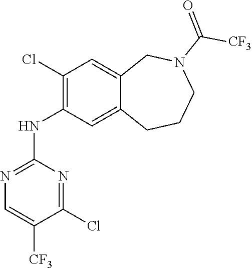 [SMILES: O=C(N1CCCc2cc(Nc3ncc(C(F)(F)F)c(Cl)n3)c(Cl)cc2C1)C(F)(F)F]

The title compound was prepared analogously to Example 1, step 5 where 1-(6-amino-7-chloro-1,2,3,4-tetrahydroisoquinolin-2-yl)-2,2,2-trifluoroethan-1-one was replaced with 1-(7-amino-8-chloro-1,3,4,5-tetrahydro-2H-benzo[c]azepin-2-yl)-2,2,2-trifluoroethan-1-one. The title compound was isolated in 90% yield. MS (ESI) m/z: 473.0 [M+H]⁺.

### Step 5: 1-(8-chloro-7-((5-(trifluoromethyl)-4-(trimethylstannyl)pyrimidin-2-yl)amino)-1,3,4,5-tetrahydro-2H-benzo[c]azepin-2-yl)-2,2,2-trifluoroethan-1-one

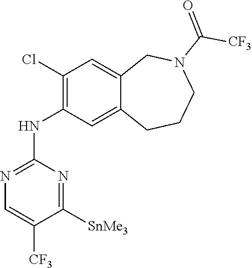

The title compound was prepared analogously to Example 4, step 1 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with 1-(8-chloro-7-((4-chloro-5-(trifluoromethyl)pyrimidin-2-yl)amino)-1,3,4,5-tetrahydro-2H-benzo[c]azepin-2-yl)-2,2,2-trifluoroethan-1-one. The title compound was isolated in 70% yield.

### Step 6: 1-(8-chloro-7-((4-(4-(methylsulfonyl)thiophen-2-yl)-5-(trifluoromethyl)pyrimidin-2-yl)amino)-1,3,4,5-tetrahydro-2H-benzo[c]azepin-2-yl)-2,2,2-trifluoroethan-1-one

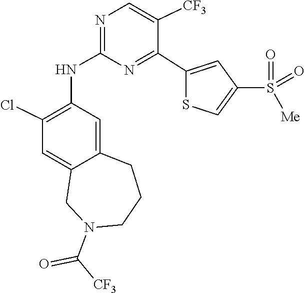 [SMILES: CS(=O)(=O)c1csc(-c2nc(Nc3cc4c(cc3Cl)CN(C(=O)C(F)(F)F)CCC4)ncc2C(F)(F)F)c1]

The title compound was prepared analogously to Example 125, step 5, where 7-bromo-3,4-dihydrothieno[2,3-f][1,4]thiazepin-5(2H)-one 1,1-dioxide and 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one were replaced with 2-bromo-4-(methylsulfonyl)thiophene and 1-(8-chloro-7-((5-(trifluoromethyl)-4-(trimethylstannyl)pyrimidin-2-yl)amino)-1,3,4,5-tetrahydro-2H-benzo[c]azepin-2-yl)-2,2,2-trifluoroethan-1-one. The title compound was isolated in 62% yield. MS (ESI) m/z: 599.1 [M+H]⁺.

### Step 7: 8-chloro-N-(4-(4-(methylsulfonyl)thiophen-2-yl)-5-(trifluoromethyl)pyrimidin-2-yl)-2,3,4,5-tetrahydro-1H-benzo[c]azepin-7-amine

A solution of 1-(8-chloro-7-((4-(4-(methylsulfonyl)thiophen-2-yl)-5-(trifluoromethyl)pyrimidin-2-yl)amino)-1,3,4,5-tetrahydro-2H-benzo[c]azepin-2-yl)-2,2,2-trifluoroethan-1-one (0.03 mmol) in ethanol (5 mL) and water (1 mL) was treated with potassium carbonate (0.25 mmol). After 10 hours, the solids were filtered and the volatiles removed under reduced pressure. The crude was purified by HPLC to afford the title compound in 7% yield. MS (ESI) m/z: 502.9 [M+H]⁺.

¹H NMR (400 MHz, CDCl₃) δ=8.77 (s, 1H), 8.31-8.30 (m, 1H), 8.24 (s, 1H), 8.08 (s, 1H), 7.79 (s, 1H), 7.21 (s, 1H), 3.91 (s, 2H), 3.29-3.20 (m, 2H), 3.16 (s, 3H), 3.06-2.95 (m, 2H), 1.85-1.74 (m, 2H)

### Example 128: N-(3-cyclopropyl-1-(1-methylpiperidin-4-yl)-1H-pyrazol-4-yl)-4-(4-(methylsulfonyl)thiophen-2-yl)-5-(trifluoromethyl)pyrimidin-2-amine

### Step 1: tert-butyl 4-(3-cyclopropyl-4-((4-(4-(methylsulfonyl)thiophen-2-yl)-5-(trifluoromethyl)pyrimidin-2-yl)amino)-1H-pyrazol-1-yl)piperidine-1-carboxylate

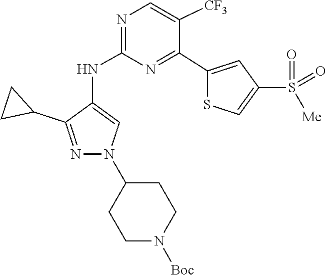 [SMILES: CC(C)(C)OC(=O)N1CCC(n2cc(Nc3ncc(C(F)(F)F)c(-c4cc(S(C)(=O)=O)cs4)n3)c(C3CC3)n2)CC1]

The title compound was prepared analogously to Example 121, step 4 where tert-butyl 3-(4-chloro-3-((4-chloro-5-(trifluoromethyl)pyrimidin-2-yl)amino) phenyl)azetidine-1-carboxylate was replaced with 4-(4-{[4-chloro-5-(trifluoromethyl)pyrimidin-2-yl]amino}-3-cyclopropyl-1H-pyrazol-1-yl)piperidine-1-carboxylate. The title compound was isolated in 96% yield. MS (ESI) m/z: 611.9 [M+H]⁺.

### Step 2: N-(3-Cyclopropyl-1-(piperidin-4-yl)-1H-pyrazol-4-yl)-4-(4-(methylsulfonyl)thiophen-2-yl)-5-(trifluoromethyl)pyrimidin-2-amine

The title compound was prepared analogously to Example 48, step 5 where tert-butyl 4-(3-cyclopropyl-4-((4-(4-oxo-4,5,6,7-tetrahydrothieno[3,2-c]pyridin-2-yl)-5-(trifluoromethyl)pyrimidin-2-yl)amino)-1H-pyrazol-1-yl)piperidine-1-carboxylate was replaced with tert-butyl 4-(3-cyclopropyl-4-((4-(4-(methylsulfonyl)thiophen-2-yl)-5-(trifluoromethyl)pyrimidin-2-yl)amino)-1H-pyrazol-1-yl)piperidine-1-carboxylate. The title compound was isolated in 84% yield. MS (ESI) m/z: 513.0 [M+H]⁺. ¹H NMR:(300 MHz, DMSO-d₆) δ 0.75 (m, 4H), 1.99 (m, 5H), 2.83 (t, J=11.7 Hz, 2H), 3.22 (m, 2H), 3.31 (s, 3H), 4.25 (s, 1H), 7.94 (m, 2H), 8.37 (s, 1H), 8.71 (m, 1H), 8.80 (m, 1H), 9.82 (m, 1H).

### Example 129: N-(3-cyclopropyl-1-(1-methylpiperidin-4-yl)-1H-pyrazol-4-yl)-4-(4-(methylsulfonyl)thiophen-2-yl)-5-(trifluoromethyl)pyrimidin-2-amine

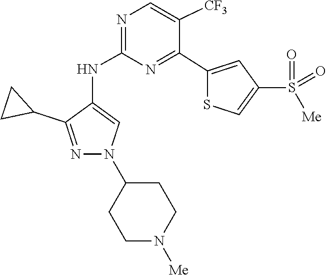 [SMILES: CN1CCC(n2cc(Nc3ncc(C(F)(F)F)c(-c4cc(S(C)(=O)=O)cs4)n3)c(C3CC3)n2)CC1]

The title compound was prepared analogously to Example 13, step 1 where methyl 5-(2-((7-ethyl-2-(2,2,2-trifluoroacetyl)-1,2,3,4-tetrahydroisoquinolin-6-yl)amino)-5-(trifluoromethyl)pyrimidin-4-yl)thiophene-3-carboxylate was replaced with N-(3-Cyclopropyl-1-(piperidin-4-yl)-1H-pyrazol-4-yl)-4-(4-(methylsulfonyl)thiophen-2-yl)-5-(trifluoromethyl)pyrimidin-2-amine. The title compound was isolated in 43% yield. MS (ESI) m/z: 527.3 [M+H]⁺. ¹H NMR (400 MHz, DMSO-d₆) δ 0.72 (m, 4H), 1.96 (m, 7H), 2.21 (s, 3H), 2.85 (d, J=10.9 Hz, 2H), 3.30 (s, 3H), 3.96 (m, 1H), 7.92 (m, 2H), 8.16 (s, 1H), 8.71 (m, 1H), 8.80 (m, 1H), 9.77 (m, 1H).

### Example 130: N-(1-(azetidin-3-yl)-3-cyclopropyl-1H-pyrazol-4-yl)-4-(4-(methylsulfonyl)thiophen-2-yl)-5-(trifluoromethyl)pyrimidin-2-amine

The title compound was prepared analogously to Example 123, where 3-methyl-4-nitro-1H-pyrazole was replaced with 3-cyclopropyl-4-nitro-1H-pyrazole in step 1. MS (ESI) m/z: 408.15 [M+H]⁺. ¹H NMR (300 MHz, DMSO-d₆) δ 10.03 (s, 1H), 9.57 (s, 1H), 9.27 (s, 1H), 8.81 (d, J=22.6 Hz, 1H), 8.70 (s, 1H), 8.05 (d, J=23.5 Hz, 1H), 7.91 (s, 1H), 5.32 (m, 1H), 4.30 (m, 4H), 3.31 (s, 3H), 2.08 (m, 1H), 0.83 (m, 4H).

### Example 131: 6-cyclopropyl-N-(4-(4-(methylsulfonyl)thiophen-2-yl)-5-(trifluoromethyl)pyrimidin-2-yl)isoindolin-5-amine

### Step 1: 1-(5-cyclopropyl-6-((4-(4-(methylsulfonyl)thiophen-2-yl)-5-(trifluoromethyl)pyrimidin-2-yl)amino)isoindolin-2-yl)-2,2,2-trifluoroethan-1-one

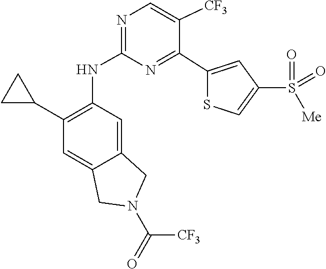 [SMILES: CS(=O)(=O)c1csc(-c2nc(Nc3cc4c(cc3C3CC3)CN(C(=O)C(F)(F)F)C4)ncc2C(F)(F)F)c1]

The title compound was prepared analogously to Example 121, step 4 where tert-butyl 3-(4-chloro-3-((4-chloro-5-(trifluoromethyl)pyrimidin-2-yl)amino) phenyl)azetidine-1-carboxylate was replaced with 1-(5-((4-chloro-5-(trifluoromethyl)pyrimidin-2-yl)amino)-6-cyclopropylisoindolin-2-yl)-2,2,2-trifluoroethan-1-one. The title compound was isolated in 72% yield. MS (ESI) m/z: 575.0 [M−H]⁻.

### Step 2: 6-cyclopropyl-N-(4-(4-(methylsulfonyl)thiophen-2-yl)-5-(trifluoromethyl)pyrimidin-2-yl)isoindolin-5-amine

The title compound was prepared analogously to Example 15, step 1 where tert-butyl-(5-(2-((7-chloro-2-(2,2,2-trifluoroacetyl)-1,2,3,4-tetrahydroisoquinolin-6-yl)amino)-5-(trifluoromethyl)pyrimidin-4-yl)thiophen-3-yl)carbamate was replaced with 1-(5-cyclopropyl-6-((4-(4-(methylsulfonyl)thiophen-2-yl)-5-(trifluoromethyl)pyrimidin-2-yl)amino)isoindolin-2-yl)-2,2,2-trifluoroethan-1-one. The title compound was isolated in 34% yield. MS (ESI) m/z: 481.15 [M+H]⁺. ¹H NMR (300 MHz, DMSO-d₆) δ 9.87 (s, 1H), 8.77 (s, 1H), 8.63 (s, 1H), 8.29 (s, 1H), 7.87 (s, 1H), 7.30 (s, 1H), 6.94 (s, 1H), 4.16 (s, 4H), 3.28 (s, 3H), 1.96 (td, J=8.5, 4.3 Hz, 1H), 0.91-0.74 (m, 2H), 0.65-0.51 (m, 2H).

### Example 132: 7-cyclopropyl-N-(4-(4-(methylsulfonyl)thiophen-2-yl)-5-(trifluoromethyl)pyrimidin-2-yl)-1,2,3,4-tetrahydroisoquinolin-6-amine

The title compound was prepared analogously to Example 131, where 1-(5-((4-chloro-5-(trifluoromethyl)pyrimidin-2-yl)amino)-6-cyclopropylisoindolin-2-yl)-2,2,2-trifluoroethan-1-one was replaced with 7-cyclopropyl-N-(4-methyl-5-(trifluoromethyl)pyrimidin-2-yl)-1,2,3,4-tetrahydroisoquinolin-6-amine. The title compound was isolated. MS (ESI) m/z: 495.0 [M+H]⁺. ¹H NMR (400 MHz, DMSO-d₆) δ 0.54 (m, 2H), 0.79 (m, 2H), 1.92 (m, 1H), 2.44 (brs, 1H), 2.64 (t, J=5.8 Hz, 2H), 2.91 (t, J=5.8 Hz, 2H), 3.28 (s, 3H), 3.79 (s, 2H), 6.63 (s, 1H), 7.10 (brs, 1H), 7.87 (s, 1H), 8.64 (s, 1H), 8.77 (s, 1H), 9.75 (s, 1H).

### Example 133: 8-chloro-2-methyl-N-(4-(4-(methylsulfonyl)thiophen-2-yl)-5-(trifluoromethyl)pyrimidin-2-yl)-2,3,4,5-tetrahydro-1H-benzo[c]azepin-7-amine

The title compound was prepared analogously to Example 17, where 5-(2-((6-chloroisoindolin-5-yl)amino)-5-(trifluoromethyl)pyrimidin-4-yl)thiophene-3-carboxamide was replaced with 8-chloro-N-(4-(4-(methylsulfonyl)thiophen-2-yl)-5-(trifluoromethyl)pyrimidin-2-yl)-2,3,4,5-tetrahydro-1H-benzo[c]azepin-7-amine. The title compound was isolated in 32% yield. MS (ESI) m/z: 517.1 [M+H]⁺. ¹H NMR (400 MHz, CDCl₃) δ=8.78 (s, 1H), 8.31 (d, J=1.2 Hz, 1H), 8.27 (s, 1H), 8.09 (s, 1H), 7.82 (s, 1H), 7.24 (s, 1H), 3.83 (s, 2H), 3.16 (s, 3H), 3.09 (d, J=2.4 Hz, 2H), 3.00-2.85 (m, 2H), 2.38 (s, 3H), 1.86 (s, 2H).

### Example 134: 7-(2-((7-fluoro-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 4, where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with 1-(6-((4-chloro-5-(trifluoromethyl)pyrimidin-2-yl)amino)-7-fluoro-3,4-dihydroisoquinolin-2(1H)-yl)-2,2,2-trifluoroethan-1-one in step 1. The title compound was isolated. MS (ESI) m/z: 626.0 [M+H]⁺. ¹H NMR (400 MHz, DMSO-d₆) δ 10.29 (s, 1H), 9.13-8.97 (m, 2H), 8.92-8.84 (m, 2H), 7.89 (s, 1H), 7.57-7.50 (m, 1H), 7.28-7.23 (m, 1H), 4.31-4.27 (m, 2H), 3.93-3.89 (m, 2H), 3.70-3.65 (m, 2H), 3.43-3.39 (m, 2H), 3.02-2.97 (m, 2H).

### Example 135: 7-(2-((7-chloro-1,2,3,4-tetrahydroisoquinolin-6-yl)amino)-5-(trifluoromethyl)pyrimidin-4-yl)-4-ethyl-3,4-dihydrothieno[2,3-f][1,4]thiazepin-5(2H)-one 1,1-dioxide

### Step 1: 7-Bromo-4-ethyl-3,4-dihydrothieno[2,3-f][1,4]thiazepin-5(2H)-one 1,1-dioxide

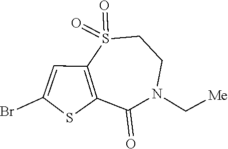 [SMILES: CCN1CCS(=O)(=O)c2cc(Br)sc2C1=O]

A 0° C. solution of 7-bromo-3,4-dihydrothieno[2,3-f][1,4]thiazepin-5(2H)-one 1,1-dioxide (0.20 mmol) in DMF (3 mL) was treated with 60% NaH in mineral oil (0.37 mmol). After 30 minutes, ethyl iodide (0.20 mmol) was added and the mixture was stirred at room temperature for 12 hours. The reaction mixture was diluted with water, extracted with ethyl acetate three times and the combined organic layers were dried over anhydrous sodium sulfate, filtered, and concentrated under reduced pressure to afford a residue that was purified by silica gel chromatography (50% ethyl acetate in hexanes). The title compound was isolated in 55% yield. MS (ESI) m/z: 323.0 [M+H]⁺.

### Step 2: 7-(2-((7-chloro-2-(2,2,2-trifluoroacetyl)-1,2,3,4-tetrahydroisoquinolin-6-yl)amino)-5-(trifluoromethyl)pyrimidin-4-yl)-4-ethyl-3,4-dihydrothieno[2,3-f][1,4]thiazepin-5(2H)-one 1,1-dioxide

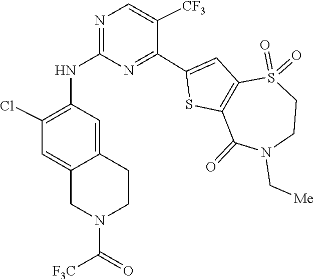 [SMILES: CCN1CCS(=O)(=O)c2cc(-c3nc(Nc4cc5c(cc4Cl)CN(C(=O)C(F)(F)F)CC5)ncc3C(F)(F)F)sc2C1=O]

The title compound was prepared analogously to Example 125, step 5 where 7-bromo-3,4-dihydrothieno[2,3-f][1,4]thiazepin-5(2H)-one 1,1-dioxide and 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one were replaced with 7-bromo-4-ethyl-1,1-dioxo-2,3-dihydrothieno[2,3-f][1,4]thiazepin-5-one and 1-[7-chloro-6-[[5-(trifluoromethyl)-4-trimethylstannyl-pyrimidin-2-yl]amino]-3,4-dihydro-1H-isoquinolin-2-yl]-2,2,2-trifluoro-ethanone. The title compound was isolated in 16% yield. MS (ESI) m/z: 668.0 [M+H]⁺.

### Step 3: 7-(2-((7-chloro-1,2,3,4-tetrahydroisoquinolin-6-yl)amino)-5-(trifluoromethyl)pyrimidin-4-yl)-4-ethyl-3,4-dihydrothieno[2,3-f][1,4]thiazepin-5(2H)-one 1,1-dioxide

The title compound was prepared analogously to Example 59, step 6 where 5-(2-((7-chloro-2-(2,2,2-trifluoroacetyl)-1,2,3,4-tetrahydroisoquinolin-6-yl)amino)-5-(trifluoromethyl)pyrimidin-4-yl)-3-(methylsulfonyl)thiophene-2-carboxamide was replaced with 7-(2-((7-chloro-2-(2,2,2-trifluoroacetyl)-1,2,3,4-tetrahydroisoquinolin-6-yl)amino)-5-(trifluoromethyl)pyrimidin-4-yl)-4-ethyl-3,4-dihydrothieno[2,3-f][1,4]thiazepin-5(2H)-one 1,1-dioxide. The title compound was isolated in 25% yield. MS (ESI) m/z: 572.0[M+H]⁺. ¹H NMR (400 MHz, DMSO-d₆) δ 10.20 (s, 1H), 9.03 (brd, J=3.2 Hz, 2H), 8.88 (brs, 1H), 7.83 (s, 1H), 7.50 (brd, J=4.6 Hz, 2H), 4.31 (s, 2H), 3.98-3.86 (m, 4H), 3.52 (d, J=7.2 Hz, 2H), 3.41 (s, 2H), 3.00 (s, 2H), 1.16 (t, J=7.1 Hz, 3H).

### Example 136: N-(5-(azetidin-3-yl)-2-methylphenyl)-4-(4-(methylsulfonyl)thiophen-2-yl)-5-(trifluoromethyl)pyrimidin-2-amine

The title compound was prepared analogously to Example 121, where 3-amino-4-chlorophenylboronic acid was replaced with 3-amino-4-methylphenylboronic acid in step 2. The title compound was isolated. MS (ESI) m/z: 481.2 [M+H]⁺. ¹H NMR (300 MHz, DMSO-d₆) δ 9.85 (s, 1H), 8.80 (s, 1H), 8.65 (d, J=1.4 Hz, 1H), 8.17 (s, 1H), 7.88 (s, 1H), 7.39 (s, 1H), 7.29-7.08 (m, 2H), 3.77 (t, J=7.4 Hz, 2H), 3.66 (t, J=7.6 Hz, 1H), 3.29 (d, J=4.4 Hz, 5H), 2.38 (s, 3H), 2.20 (s, 3H).

### Example 137: N-(2-Methyl-5-(1-methylazetidin-3-yl)phenyl)-4-(4-(methylsulfonyl)thiophen-2-yl)-5-(trifluoromethyl)pyrimidin-2-amine

The title compound was prepared analogously to Example 17, where 5-(2-((6-chloroisoindolin-5-yl)amino)-5-(trifluoromethyl)pyrimidin-4-yl)thiophene-3-carboxamide was replaced with N-(5-(azetidin-3-yl)-2-methylphenyl)-4-(4-(methylsulfonyl)thiophen-2-yl)-5-(trifluoromethyl)pyrimidin-2-amine. The title compound was isolated in 33% yield. m/z (ESI, +ve)=483.0 [M+H]⁺

¹H NMR (300 MHz, DMSO-d₆) δ 9.85 (s, 1H), 8.80 (s, 1H), 8.65 (d, J=1.4 Hz, 1H), 8.17 (s, 1H), 7.88 (s, 1H), 7.39 (s, 1H), 7.29-7.08 (m, 2H), 3.77 (t, J=7.4 Hz, 2H), 3.66 (t, J=7.6 Hz, 1H), 3.29 (d, J=4.4 Hz, 5H), 2.38 (s, 3H), 2.20 (s, 3H).

### Example 138: 7-(2-((7-ethyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

### Step 1: 1-(7-ethyl-6-nitro-3,4-dihydroisoquinolin-2(1H)-yl)-2,2,2-trifluoroethan-1-one

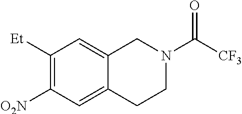 [SMILES: CCc1cc2c(cc1[N+](=O)[O-])CCN(C(=O)C(F)(F)F)C2]

A mixture of 1-(7-bromo-6-nitro-3,4-dihydro-1H-isoquinolin-2-yl)-2,2,2-trifluoro-ethanone (2.83 mmol) and (1,1′-bis(diphenylphosphino)ferrocene)palladium(II) dichloride (0.14 mmol) in dioxane (100 mL), was treated with a 1M solution of diethylzinc in hexanes (8.5 mL) and the resulting solution was stirred at 60° C. for 11 hours. The reaction was cooled down to room temperature, poured into ice-water and extracted with ethyl acetate three times. The combined organic layers were dried over anhydrous sodium sulfate, filtered, and concentrated under reduced pressure to afford a residue that was purified by silica gel chromatography (10-50% ethyl acetate in hexanes). The title compound was isolated in 30% yield. ¹H NMR (400 MHz, CDCl₃) δ 7.77-7.74 (m, 1H), 7.18-7.12 (m, 1H), 4.87-4.77 (m, 2H), 3.94-3.85 (m, 2H), 3.06-2.97 (m, 2H), 2.91 (q, J=7.6 Hz, 2H), 1.31-1.24 (m, 3H).

### Step 2: 1-(6-amino-7-ethyl-3,4-dihydroisoquinolin-2(1H)-yl)-2,2,2-trifluoroethan-1-one

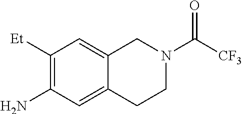 [SMILES: CCc1cc2c(cc1N)CCN(C(=O)C(F)(F)F)C2]

The title compound was prepared analogously to Example 19, step 3 where 1-(5-cyclopropyl-6-nitroisoindolin-2-yl)-2,2,2-trifluoroethan-1-one was replaced with 1-(7-ethyl-6-nitro-3,4-dihydroisoquinolin-2(1H)-yl)-2,2,2-trifluoroethan-1-one. The title compound was isolated in 95% yield. ¹H NMR (400 MHz, DMSO-d₆) δ 6.77 (d, J=3.6 Hz, 1H), 6.41 (d, J=4.4 Hz, 1H), 4.82-4.72 (m, 2H), 4.59-4.53 (m, 2H), 3.75-3.69 (m, 2H), 2.77-2.67 (m, 2H), 2.40 (q, J=7.6 Hz, 2H), 1.10 (t, J=7.6 Hz, 3H).

### Step 3: 1-(6-((4-chloro-5-(trifluoromethyl)pyrimidin-2-yl)amino)-7-ethyl-3,4-dihydroisoquinolin-2(1H)-yl)-2,2,2-trifluoroethan-1-one

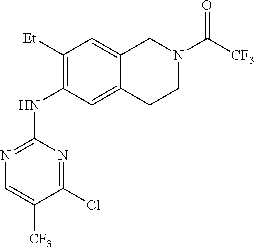 [SMILES: CCc1cc2c(cc1Nc1ncc(C(F)(F)F)c(Cl)n1)CCN(C(=O)C(F)(F)F)C2]

The title compound was prepared analogously to Example 1, step 5 where 1-(6-amino-7-chloro-1,2,3,4-tetrahydroisoquinolin-2-yl)-2,2,2-trifluoroethan-1-one was replaced with 1-(6-amino-7-ethyl-3,4-dihydroisoquinolin-2(1H)-yl)-2,2,2-trifluoroethan-1-one. The title compound was isolated in 43% yield. MS (ESI) m/z: 453.1 [M+H]⁺. ¹H NMR (400 MHz, CDCl₃) δ 8.55-8.51 (m, 1H), 7.58-7.52 (m, 1H), 7.16 (s, 1H), 7.10-7.03 (m, 1H), 4.81-4.74 (m, 2H), 3.92-3.83 (m, 2H), 3.00-2.93 (m, 2H), 2.70-2.59 (m, 2H), 1.26-1.21 (m, 3H).

### Step 4: 1-(7-ethyl-6-((5-(trifluoromethyl)-4-(trimethylstannyl)pyrimidin-2-yl)amino)-3,4-dihydroisoquinolin-2(1H)-yl)-2,2,2-trifluoroethan-1-one

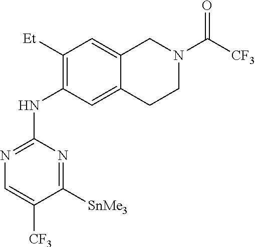

The title compound was prepared analogously to Example 4, step 1 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one (3.26 mmol), 1,4-bis(diphenylphosphino)butane was replaced with 1-(6-((4-chloro-5-(trifluoromethyl)pyrimidin-2-yl)amino)-7-ethyl-3,4-dihydroisoquinolin-2(1H)-yl)-2,2,2-trifluoroethan-1-one. The title compound was isolated in 23% yield. MS (ESI) m/z: 583.2 [M+H]⁺.

### Step 5: 7-(2-((7-ethyl-2-(2,2,2-trifluoroacetyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

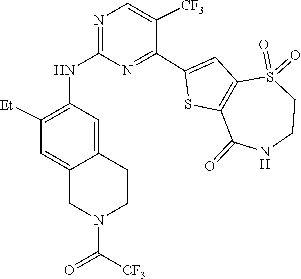 [SMILES: CCc1cc2c(cc1Nc1ncc(C(F)(F)F)c(-c3cc4c(s3)C(=O)NCCS4(=O)=O)n1)CCN(C(=O)C(F)(F)F)C2]

The title compound was prepared analogously to Example 125, step 5 where 7-bromo-3,4-dihydrothieno[2,3-f][1,4]thiazepin-5(2H)-one 1,1-dioxide and 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one were replaced with 7-bromo-3,4-dihydrothieno[2,3-f][1,4]thiazepin-5(2H)-one 1,1-dioxide and 1-(7-ethyl-6-((5-(trifluoromethyl)-4-(trimethylstannyl)pyrimidin-2-yl)amino)-3,4-dihydroisoquinolin-2(1H)-yl)-2,2,2-trifluoroethan-1-one. The title compound was isolated in 70% yield. MS (ESI) m/z: 634.1 [M+H]⁺.

### Step 6: 7-(2-((7-ethyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 59, step 6 where 5-(2-((7-chloro-2-(2,2,2-trifluoroacetyl)-1,2,3,4-tetrahydroisoquinolin-6-yl)amino)-5-(trifluoromethyl)pyrimidin-4-yl)-3-(methylsulfonyl)thiophene-2-carboxamide was replaced with 7-(2-((7-ethyl-2-(2,2,2-trifluoroacetyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide. The title compound was isolated in 55% yield. MS (ESI) m/z: 538.1[M+H]⁺. ¹H NMR (400 MHz, methanol-d₄) δ 8.74 (s, 1H), 8.53 (d, J=1.6 Hz, 1H), 8.05 (s, 1H), 7.47 (s, 1H), 7.17 (s, 1H), 4.35 (s, 2H), 3.79 (s, 4H), 3.50 (t, J=6.4 Hz, 2H), 3.14 (t, J=6.0 Hz, 2H), 2.69 (q, J=7.6 Hz, 2H), 1.19 (t, J=7.6 Hz, 3H).

### Example 139: 7-(2-((7-fluoro-2-methyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 7-(2-((7-fluoro-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide. The title compound was isolated in 11% yield. m/z (ESI, +ve)=542.1 [M+H]⁺. ¹H NMR (400 MHz, DMSO-d₆) δ 10.31 (s, 1H), 10.18-9.95 (m, 1H), 8.96-8.81 (m, 2H), 7.91-7.83 (m, 1H), 7.60-7.50 (m, 1H), 7.28-7.21 (m, 1H), 4.60-4.20 (m, 2H), 3.95-3.89 (m, 2H), 3.75-3.60 (m, 2H), 3.30-3.21 (m, 2H), 3.12-3.05 (m, 2H), 2.94 (s, 3H).

### Example 140: 7-(2-((7-fluoro-2-methyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

### Step 1: 7-Bromo-4-methyl-3,4-dihydrothieno[2,3-f][1,4]thiazepin-5(2H)-one 1,1-dioxide

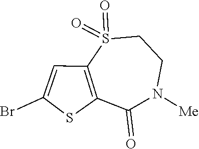 [SMILES: CN1CCS(=O)(=O)c2cc(Br)sc2C1=O]

The title compound was prepared analogously to Example 135, step 1 where ethyl iodide was replaced with methyl iodide. The title compound was isolated in 64% yield. m/z (ESI, +ve)=310.0 [M+H]⁺. ¹H NMR (400 MHz, DMSO-d₆) δ 7.61 (s, 1H), 3.98-3.93 (m, 2H), 3.93-3.88 (m, 2H), 3.05 (s, 3H).

### Step 2: 7-(2-((7-fluoro-2-(2,2,2-trifluoroacetyl)-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

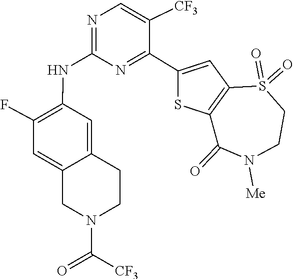 [SMILES: CN1CCS(=O)(=O)c2cc(-c3nc(Nc4cc5c(cc4F)CN(C(=O)C(F)(F)F)CC5)ncc3C(F)(F)F)sc2C1=O]

The title compound was prepared analogously to Example 125, step 5 where 7-bromo-3,4-dihydrothieno[2,3-f][1,4]thiazepin-5(2H)-one 1,1-dioxide and 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one were replaced with 7-Bromo-4-methyl-3,4-dihydrothieno[2,3-f][1,4]thiazepin-5(2H)-one 1,1-dioxide and 2,2,2-trifluoro-1-(7-fluoro-6-((5-(trifluoromethyl)-4-(trimethylstannyl)pyrimidin-2-yl)amino)-3,4-dihydroisoquinolin-2(1H)-yl)ethan-1-one. The title compound was isolated in 75% yield. MS (ESI) m/z: 638.1 [M+H]⁺.

### Step 3: 7-(2-((7-fluoro-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

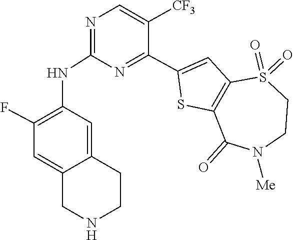 [SMILES: CN1CCS(=O)(=O)c2cc(-c3nc(Nc4cc5c(cc4F)CNCC5)ncc3C(F)(F)F)sc2C1=O]

The title compound was prepared analogously to Example 59, step 6 where 5-(2-((7-chloro-2-(2,2,2-trifluoroacetyl)-1,2,3,4-tetrahydroisoquinolin-6-yl)amino)-5-(trifluoromethyl)pyrimidin-4-yl)-3-(methylsulfonyl)thiophene-2-carboxamide was replaced with -(2-((7-fluoro-2-(2,2,2-trifluoroacetyl)-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 64% yield. MS (ESI) m/z: 542.1 [M+H]⁺.

### Step 4: 7-(2-((7-fluoro-2-methyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 7-(2-((7-fluoro-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 22% yield. m/z (ESI, +ve)=556.1 [M+H]⁺. ¹H NMR (400 MHz, DMSO-d₆) δ 10.31 (br s, 1H), 10.07 (br s, 1H), 8.91 (s, 1H), 7.85 (s, 1H), 7.54 (d, J=7.6 Hz, 1H), 7.25 (d, J=10.8 Hz, 1H), 4.59-4.42 (m, 1H), 4.38-4.21 (m, 1H), 4.01-3.94 (m, 2H), 3.93-3.86 (m, 2H), 3.77-3.63 (m, 1H), 3.44-3.39 (m, 1H), 3.14-3.03 (m, 5H), 2.96 (s, 3H).

### Example 141: 7-(2-((3-cyclopropyl-1-(piperidin-4-yl)-1H-pyrazol-4-yl)amino)-5-(trifluoromethyl)pyrimidin-4-yl)-4-methyl-3,4-dihydrothieno[2,3-f][1,4]thiazepin-5(2H)-one 1,1-dioxide

### Step 1: tert-butyl 4-(3-cyclopropyl-4-((5-(trifluoromethyl)-4-(trimethylstannyl)pyrimidin-2-yl)amino)-1H-pyrazol-1-yl)piperidine-1-carboxylate

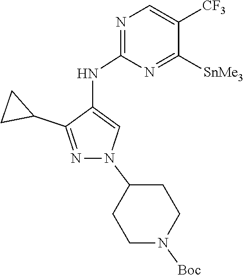

The title compound was prepared analogously to Example 80, step 3 where 1-(5-((4-chloro-5-(trifluoromethyl)pyrimidin-2-yl)amino)-6-fluoroisoindolin-2-yl)-2,2,2-trifluoroethan-1-one was replaced with tert-butyl 4-(4-((4-chloro-5-(trifluoromethyl)pyrimidin-2-yl)amino)-3-cyclopropyl-1H-pyrazol-1-yl)piperidine-1-carboxylate. The title compound was isolated in 28% yield. m/z (ESI, +ve)=617.2 [M+H]⁺.

### Step 2: tert-butyl 4-(3-cyclopropyl-4-((4-(4-methyl-1,1-dioxido-5-oxo-2,3,4,5-tetrahydrothieno[2,3-f][1,4]thiazepin-7-yl)-5-(trifluoromethyl)pyrimidin-2-yl)amino)-1H-pyrazol-1-yl)piperidine-1-carboxylate

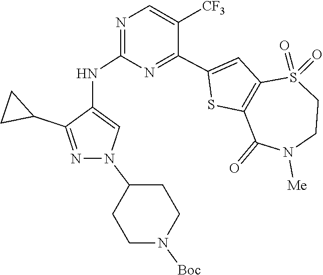 [SMILES: CN1CCS(=O)(=O)c2cc(-c3nc(Nc4cn(C5CCN(C(=O)OC(C)(C)C)CC5)nc4C4CC4)ncc3C(F)(F)F)sc2C1=O]

The title compound was prepared analogously to Example 125, step 5 where 7-bromo-3,4-dihydrothieno[2,3-f][1,4]thiazepin-5(2H)-one 1,1-dioxide and 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one were replaced with 7-bromo-4-methyl-3,4-dihydrothieno[2,3-f][1,4]thiazepin-5(2H)-one 1,1-dioxide and tert-butyl 4-(3-cyclopropyl-4-((5-(trifluoromethyl)-4-(trimethylstannyl)pyrimidin-2-yl)amino)-1H-pyrazol-1-yl)piperidine-1-carboxylate. The title compound was isolated in 48% yield. m/z: 682.1 [M+H]⁺.

### Step 3: 7-(2-((3-cyclopropyl-1-(piperidin-4-yl)-1H-pyrazol-4-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 64, step 2 where methyl 3-((2-((tert-butoxycarbonyl)amino)ethyl)sulfonyl)thiophene-2-carboxylate was replaced with tert-butyl 4-(3-cyclopropyl-4-((4-(4-methyl-1,1-dioxido-5-oxo-2,3,4,5-tetrahydrothieno[2,3-f][1,4]thiazepin-7-yl)-5-(trifluoromethyl)pyrimidin-2-yl)amino)-1H-pyrazol-1-yl)piperidine-1-carboxylate. The title compound was isolated in 48% yield. m/z: 582.1 [M+H]⁺. ¹H NMR (400 MHz, methanol-d₄) δ 8.77 (s, 1H), 8.14 (s, 1H), 8.01 (s, 1H), 4.49-4.44 (m, 1H), 4.00-3.98 (m, 2H), 3.89 (t, J=5.6 Hz, 2H), 3.59 (d, J=12.8 Hz, 2H), 3.28-3.24 (m, 2H), 3.21 (s, 3H), 2.42-2.39 (m, 2H), 2.27 (d, J=10.8 Hz, 2H), 1.96-1.91 (m, 1H), 0.91 (d, J=8.4 Hz, 2H), 0.82 (d, J=3.2 Hz, 2H).

### Example 142: 7-(2-((7-chloro-2-methyl-1,2,3,4-tetrahydroisoquinolin-6-yl)amino)-5-(trifluoromethyl)pyrimidin-4-yl)-4-ethyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 7-(2-((7-chloro-1,2,3,4-tetrahydroisoquinolin-6-yl)amino)-5-(trifluoromethyl)pyrimidin-4-yl)-4-ethyl-3,4-dihydrothieno[2,3-f][1,4]thiazepin-5(2H)-one 1,1-dioxide. The title compound was isolated. m/z (ESI, +ve)=586.1 [M+H]⁺. ¹H NMR (400 MHz, DMSO-d₆) δ 10.22 (s, 1H), 10.02-9.84 (m, 1H), 8.91-8.85 (m, 1H), 7.84 (s, 1H), 7.56-7.51 (m, 1H), 7.49 (s, 1H), 3.96-3.86 (m, 4H), 3.55-3.49 (m, 2H), 3.12-3.04 (m, 2H), 2.99-2.89 (m, 3H), 2.57-2.54 (m, 4H), 1.16 (t, J=7.6 Hz, 3H).

### Example 143: 7-(2-((7-chloro-1,2,3,4-tetrahydroisoquinolin-6-yl)amino)-5-(trifluoromethyl)pyrimidin-4-yl)-4-(2,2,2-trifluoroethyl)-3,4-dihydrothieno[2,3-f][1,4]thiazepin-5(2H)-one 1,1-dioxide

### Step 1: 7-bromo-4-(2,2,2-trifluoroethyl)-3,4-dihydrothieno[2,3-f][1,4]thiazepin-5(2H)-one 1,1-dioxide

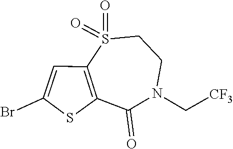 [SMILES: O=C1c2sc(Br)cc2S(=O)(=O)CCN1CC(F)(F)F]

The title compound was prepared analogously to Example 158, step 1 where bromocyclobutane was replaced with 2,2,2-trifluoroethyl trifluoromethanesulfonate. The title compound was isolated in 68% yield. MS (ESI) m/z: 377.9 [M+H]⁺.

### Step 2: 7-(2-((7-chloro-1,2,3,4-tetrahydroisoquinolin-6-yl)amino)-5-(trifluoromethyl)pyrimidin-4-yl)-4-(2,2,2-trifluoroethyl)-3,4-dihydrothieno[2,3-f][1,4]thiazepin-5(2H)-one 1,1-dioxide

The title compound was prepared analogously to Example 135, where ethyl iodide was replaced with 2,2,2-trifluoroethyl trifluoromethanesulfonate in step 1, and 7-bromo-4-ethyl-1,1-dioxo-2,3-dihydrothieno[2,3-f][1,4]thiazepin-5-one was replaced with 7-bromo-4-(2,2,2-trifluoroethyl)-3,4-dihydrothieno[2,3-f][1,4]thiazepin-5(2H)-one 1,1-dioxide in step 2. The title compound was isolated. m/z (ESI, +ve)=626.0 [M+H]⁺. ¹H NMR (400 MHz, DMSO-d₆) δ 10.23 (s, 1H), 9.15-8.99 (m, 1H), 8.90 (s, 1H), 7.86 (s, 1H), 7.53-7.48 (m, 2H), 4.48-4.40 (m, 2H), 4.34-4.28 (m, 2H), 4.12-4.04 (m, 2H), 3.98-3.92 (m, 2H), 3.45-3.41 (m, 2H), 3.04-2.97 (m, 2H).

### Example 144: 7-(2-((7-chloro-2-methyl-1,2,3,4-tetrahydroisoquinolin-6-yl)amino)-5-(trifluoromethyl)pyrimidin-4-yl)-4-(2,2,2-trifluoroethyl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 7-(2-((7-chloro-1,2,3,4-tetrahydroisoquinolin-6-yl)amino)-5-(trifluoromethyl)pyrimidin-4-yl)-4-(2,2,2-trifluoroethyl)-3,4-dihydrothieno[2,3-f][1,4]thiazepin-5(2H)-one 1,1-dioxide. The title compound was isolated in 43% yield. m/z (ESI, +ve)=640.1 [M+H]⁺. ¹H NMR (400 MHz, DMSO-d₆) δ 10.27 (s, 1H), 10.00 (br s, 1H), 8.91 (s, 1H), 7.86 (s, 1H), 7.54-7.49 (m, 2H), 4.59-4.50 (m, 1H), 4.48-4.40 (m, 2H), 4.37-4.27 (m, 1H), 4.11-4.05 (m, 2H), 3.98-3.92 (m, 2H), 3.75-3.65 (m, 1H), 3.46-3.41 (m, 1H), 3.13-3.04 (m, 2H), 2.96 (s, 3H).

### Example 145: N-(3-cyclopropyl-1-(1-methylazetidin-3-yl)-1H-pyrazol-4-yl)-4-(4-(methylsulfonyl)thiophen-2-yl)-5-(trifluoromethyl)pyrimidin-2-amine

The title compound was prepared analogously to Example 17, where 5-(2-((6-chloroisoindolin-5-yl)amino)-5-(trifluoromethyl)pyrimidin-4-yl)thiophene-3-carboxamide was replaced with N-(1-(azetidin-3-yl)-3-cyclopropyl-1H-pyrazol-4-yl)-4-(4-(methylsulfonyl)thiophen-2-yl)-5-(trifluoromethyl)pyrimidin-2-amine. The title compound was isolated in 9% yield. m/z (ESI, +ve)=499.5 [M+H]⁺. ¹H NMR (300 MHz, DMSO-d₆) δ 9.85 (d, J=82.2 Hz, 1H), 8.88-8.67 (m, 2H), 8.16 (s, 1H), 7.94 (d, J=14.3 Hz, 1H), 4.83 (q, J=6.8 Hz, 1H), 3.70 (t, J=7.3 Hz, 3H), 3.31 (s, 3H), 2.35 (s, 3H), 2.09-1.86 (m, 1H), 0.87-0.68 (m, 4H).

### Example 146: 7-(2-((7-ethyl-2-methyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 7-(2-((7-ethyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide. The title compound was isolated in 45% yield. m/z (ESI, +ve)=552.2 [M+H]⁺. ¹H NMR (400 MHz, methanol-d₄) δ 8.71 (s, 1H), 8.52 (s, 1H), 8.06-7.98 (m, 1H), 7.36 (d, J=5.2 Hz, 1H), 7.07 (s, 1H), 3.93 (s, 2H), 3.78 (s, 4H), 3.07 (s, 4H), 2.72-2.60 (m, 5H), 1.21-1.14 (m, 3H).

### Example 147: 7-(2-((7-ethyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 138, where 7-bromo-3,4-dihydrothieno[2,3-f][1,4]thiazepin-5(2H)-one 1,1-dioxide was replaced with 7-bromo-4-methyl-3,4-dihydrothieno[2,3-f][1,4]thiazepin-5(2H)-one 1,1-dioxide in step 5. The title compound was isolated in 44% yield. m/z (ESI, +ve)=552.3 [M+H]⁺. ¹H NMR (400 MHz, methanol-d₄) δ 8.73 (s, 1H), 7.99 (s, 1H), 7.43 (s, 1H), 7.16 (s, 1H), 4.32 (s, 2H), 3.98-3.90 (m, 2H), 3.89-3.81 (m, 2H), 3.47 (t, J=5.6 Hz, 2H), 3.19 (s, 3H), 3.11 (s, 2H), 2.68 (q, J=7.6 Hz, 2H), 1.18 (t, J=7.6 Hz, 3H).

### Example 148: 7-(2-((7-ethyl-2-methyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 7-(2-((7-ethyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 69% yield. m/z (ESI, +ve)=566.2 [M+H]⁺. ¹H NMR (400 MHz, methanol-d₄) δ 8.70 (s, 1H), 8.51 (s, 1H), 7.98 (s, 1H), 7.34 (s, 1H), 7.07 (s, 1H), 3.92 (s, 4H), 3.87-3.81 (m, 2H), 3.18 (s, 3H), 3.06 (s, 4H), 2.70-2.61 (m, 5H), 1.17 (t, J=7.6 Hz, 3H).

### Example 149: 7-(2-((2-chloro-5-(piperidin-4-yl)phenyl)amino)-5-(trifluoromethyl)pyrimidin-4-yl)-4-methyl-3,4-dihydrothieno[2,3-f][1,4]thiazepin-5(2H)-one 1,1-dioxide

### Step 1: tert-butyl 4-(3-amino-4-chlorophenyl)-3,6-dihydropyridine-1(2H)-carboxylate

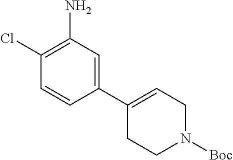 [SMILES: CC(C)(C)OC(=O)N1CC=C(c2ccc(Cl)c(N)c2)CC1]

A solution of 5-bromo-2-chloroaniline (0.48 mmol), tert-butyl 4-(4,4,5,5-tetramethyl-1,3,2-dioxaborolan-2-yl)-3,6-dihydropyridine-1(2H)-carboxylate (0.58 mmol), bis-triphenylphosphine-palladium(II) chloride (0.05 mmol), sodium carbonate (2.4 mmol) in DMF (2 mL) and water (1.2 mL) was refluxed for 1 hour. The reaction was cooled down to room temperature and concentrated. The crude product was purified by preparative TLC (30% ethyl acetate in hexanes) to afford the title compound in 47% yield. MS (ESI) m/z: 309.0 [M+H]⁺. ¹H NMR (400 MHz, DMSO-d₆) δ 7.07 (d, J=8.0 Hz, 1H), 6.65 (d, J=2.0 Hz, 1H), 6.42 (dd, J=8.4, 2.0 Hz, 1H), 2.82-2.72 (m, 3H), 1.77-1.67 (m, 4H), 1.41 (s, 9H).

### Step 2: tert-butyl 4-(3-amino-4-chlorophenyl)piperidine-1-carboxylate

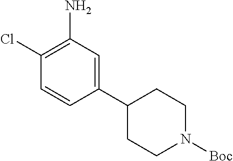 [SMILES: CC(C)(C)OC(=O)N1CCC(c2ccc(Cl)c(N)c2)CC1]

A solution of tert-butyl 4-(3-amino-4-chlorophenyl)-3,6-dihydropyridine-1(2H)-carboxylate (9.71 mmol) in methanol (60 mL) was hydrogenated for 15 minutes in the presence of 10% Pd on carbon (600 mg). After filtration through celite and evaporation of volatiles, the crude product was purified by silica gel chromatography (10-20% ethyl acetate in hexanes) to afford the title compound in 50% yield.

### Step 3: tert-butyl 4-(4-chloro-3-((4-chloro-5-(trifluoromethyl)pyrimidin-2-yl)amino)phenyl)piperidine-1-carboxylate

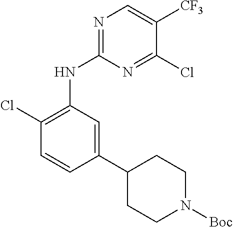 [SMILES: CC(C)(C)OC(=O)N1CCC(c2ccc(Cl)c(Nc3ncc(C(F)(F)F)c(Cl)n3)c2)CC1]

The title compound was prepared analogously to tert-butyl 3-(4-chloro-3-((4-chloro-5-(trifluoromethyl)pyrimidin-2-yl)amino) phenyl)azetidine-1-carboxylate where tert-butyl 3-(3-amino-4-chlorophenyl)azetidine-1-carboxylate was replaced with tert-butyl 4-(3-amino-4-chlorophenyl)piperidine-1-carboxylate. The title compound was isolated in 63% yield.

### Step 4: tert-butyl 4-(4-chloro-3-((5-(trifluoromethyl)-4-(trimethylstannyl)pyrimidin-2-yl)amino)phenyl)piperidine-1-carboxylate

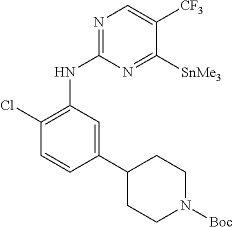

The title compound was prepared analogously to Example 4, step 1 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-on was replaced with tert-butyl 4-(4-chloro-3-((4-chloro-5-(trifluoromethyl)pyrimidin-2-yl)amino)phenyl)piperidine-1-carboxylate. The title compound was isolated in 53% yield.

### Step 5: 7-(2-((2-chloro-5-(piperidin-4-yl)phenyl)amino)-5-(trifluoromethyl)pyrimidin-4-yl)-4-methyl-3,4-dihydrothieno[2,3-f][1,4]thiazepin-5(2H)-one 1,1-dioxide

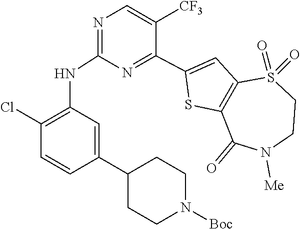 [SMILES: CN1CCS(=O)(=O)c2cc(-c3nc(Nc4cc(C5CCN(C(=O)OC(C)(C)C)CC5)ccc4Cl)ncc3C(F)(F)F)sc2C1=O]

The title compound was prepared analogously to Example 125, step 5, where 7-bromo-3,4-dihydrothieno[2,3-f][1,4]thiazepin-5(2H)-one 1,1-dioxide and 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one were replaced with tert-butyl 4-(4-chloro-3-((5-(trifluoromethyl)-4-(trimethylstannyl)pyrimidin-2-yl)amino)phenyl)piperidine-1-carboxylate and 7-bromo-4-methyl-3,4-dihydrothieno[2,3-f][1,4]thiazepin-5(2H)-one 1,1-dioxide. The title compound was isolated in 53% yield. MS (ESI) m/z: 585.9 [M+H-Boc]⁺.

### Step 6: 7-(2-((2-chloro-5-(piperidin-4-yl)phenyl)amino)-5-(trifluoromethyl)pyrimidin-4-yl)-4-methyl-3,4-dihydrothieno[2,3-f][1,4]thiazepin-5(2H)-one 1,1-dioxide

A solution of 7-(2-((2-chloro-5-(piperidin-4-yl)phenyl)amino)-5-(trifluoromethyl)pyrimidin-4-yl)-4-methyl-3,4-dihydrothieno[2,3-f][1,4]thiazepin-5(2H)-one 1,1-dioxide (0.10 mmol) in dichloromethane (2 mL) and TFA (1 mL) was stirred for one hour. Evaporation of the volatiles under reduced pressure and purification of the resulting crude material by HPLC, afforded the title compound in 3% yield. MS (ESI) m/z: 585.9 [M+H]⁺. ¹H NMR (400 MHz, methanol-d₄) δ 8.86 (s, 1H), 8.11 (s, 1H), 8.05 (s, 1H), 7.48 (d, J=8.0 Hz, 1H), 7.12 (dd, J=8.4, 2.0 Hz, 1H), 3.99-3.96 (m, 2H), 3.90-3.87 (m, 2H), 3.57 (d, J=12.4 Hz, 2H), 3.23-3.17 (m, 5H), 3.07-3.03 (m, 1H), 2.20-2.16 (m, 2H), 2.04-2.00 (m, 2H).

### Example 150: 7-(2-((2-chloro-5-(1-methylpiperidin-4-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with -(2-((2-chloro-5-(piperidin-4-yl)phenyl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 54% yield. m/z (ESI, +ve)=598.0 [M+H]⁺. ¹H NMR (400 MHz, methanol-d₄) δ 8.87 (s, 1H), 8.16 (s, 1H), 8.06 (s, 1H), 7.77-7.73 (m, 1H), 7.48 (d, J=8.4 Hz, 1H), 7.12 (dd, J=8.4, 2.0 Hz, 1H), 4.01-3.98 (m, 2H), 3.91-3.88 (m, 2H), 3.68 (d, J=12.4 Hz, 2H), 3.22-3.19 (m, 5H), 3.01-2.96 (m, 1H), 2.96 (s, 3H), 2.24-2.21 (m, 2H), 2.11-2.08 (m, 2H).

### Example 151: 7-(2-((3-cyclopropyl-1-(piperidin-4-yl)-1H-pyrazol-4-yl)amino)-5-(trifluoromethyl)pyrimidin-4-yl)-3,4-dihydrothieno[2,3-f][1,4]thiazepin-5(2H)-one 1,1-dioxide

### Step 1: tert-butyl 4-(3-cyclopropyl-4-((4-(1,1-dioxido-5-oxo-2,3,4,5-tetrahydrothieno[2,3-f][1,4]thiazepin-7-yl)-5-(trifluoromethyl)pyrimidin-2-yl)amino)-1H-pyrazol-1-yl)piperidine-1-carboxylate

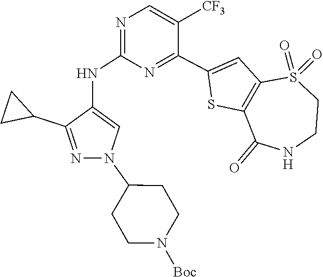 [SMILES: CC(C)(C)OC(=O)N1CCC(n2cc(Nc3ncc(C(F)(F)F)c(-c4cc5c(s4)C(=O)NCCS5(=O)=O)n3)c(C3CC3)n2)CC1]

The title compound was prepared analogously to Example 141, where 7-bromo-4-methyl-3,4-dihydrothieno[2,3-f][1,4]thiazepin-5(2H)-one 1,1-dioxide was replaced with 7-bromo-3,4-dihydrothieno[2,3-f][1,4]thiazepin-5(2H)-one 1,1-dioxide. The title compound was isolated in 47% yield.

### Step 2: 7-(2-((3-cyclopropyl-1-(piperidin-4-yl)-1H-pyrazol-4-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 149, step 5 where 7-(2-((2-chloro-5-(piperidin-4-yl)phenyl)amino)-5-(trifluoromethyl)pyrimidin-4-yl)-4-methyl-3,4-dihydrothieno[2,3-f][1,4]thiazepin-5(2H)-one 1,1-dioxide was replaced with tert-butyl 4-(3-cyclopropyl-4-((4-(1,1-dioxido-5-oxo-2,3,4,5-tetrahydrothieno[2,3-f][1,4]thiazepin-7-yl)-5-(trifluoromethyl)pyrimidin-2-yl)amino)-1H-pyrazol-1-yl)piperidine-1-carboxylate. The title compound was isolated in 88% yield. m/z (ESI, +ve)=568.1 [M+H]⁺. ¹H NMR (400 MHz, methanol-d₄) δ 8.72 (s, 1H), 8.14 (s, 1H), 8.04 (s, 1H), 4.46-4.41 (m, 1H), 3.83 (s, 4H), 3.58 (d, J=12.8 Hz, 2H), 3.23 (t, J=12.4 Hz, 2H), 2.41-2.22 (m, 4H), 1.96-1.91 (m, 1H), 0.92-0.82 (m, 4H).

### Example 152: 7-(2-((3-cyclopropyl-1-(1-methylpiperidin-4-yl)-1H-pyrazol-4-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 7-(2-((3-cyclopropyl-1-(piperidin-4-yl)-1H-pyrazol-4-yl)amino)-5-(trifluoromethyl)pyrimidin-4-yl)-3,4-dihydrothieno[2,3-f][1,4]thiazepin-5(2H)-one 1,1-dioxide. The title compound was isolated in 38% yield. m/z (ESI, +ve)=582.3 [M+H]⁺. ¹H NMR (400 MHz, methanol-d₄) δ 8.75 (s, 1H), 8.51 (s, 0.9H), 8.13 (s, 1H), 8.06 (s, 1H), 4.32-4.26 (m, 1H), 3.81 (s, 4H), 3.38 (d, J=11.6 Hz, 2H), 2.85 (t, J=11.2 Hz, 2H), 2.69 (s, 3H), 2.35-2.27 (m, 2H), 2.25-2.18 (m, 2H), 1.94-1.89 (m, 1H), 0.91-0.89 (m, 2H), 0.82 (d, J=2.8 Hz, 2H).

### Example 153: 7-(2-((7-chloro-1,2,3,4-tetrahydroisoquinolin-6-yl)amino)-5-(trifluoromethyl)pyrimidin-4-yl)-4-(cyclopropylmethyl)-3,4-dihydrothieno[2,3-f][1,4]thiazepin-5(2H)-one 1,1-dioxide

### Step 1: 7-Bromo-4-(cyclopropylmethyl)-3,4-dihydrothieno[2,3-f][1,4]thiazepin-5(2H)-one 1,1-dioxide

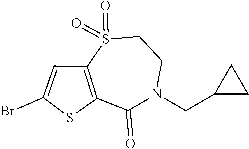 [SMILES: O=C1c2sc(Br)cc2S(=O)(=O)CCN1CC1CC1]

The title compound was prepared analogously to Example 135, step 1 where ethyl iodide was replaced with (bromomethyl)cyclopropane. The title compound was isolated in 52% yield. ¹H NMR (400 MHz, DMSO-d₆) δ 7.61 (s, 1H), 4.00-3.95 (m, 2H), 3.94-3.90 (m, 2H), 3.48-3.41 (m, 1H), 3.30-3.26 (m, 1H), 1.10-0.99 (m, 1H), 0.54-0.47 (m, 2H), 0.32-0.27 (m, 2H).

### Steps 2-3: 7-(2-((7-chloro-1,2,3,4-tetrahydroisoquinolin-6-yl)amino)-5-(trifluoromethyl)pyrimidin-4-yl)-4-(cyclopropylmethyl)-3,4-dihydrothieno[2,3-f][1,4]thiazepin-5(2H)-one 1,1-dioxide

The title compound was prepared analogously to Example 135, steps 2-3, where 7-bromo-3,4-dihydrothieno[2,3-f][1,4]thiazepin-5(2H)-one 1,1-dioxide was replaced with 7-bromo-4-(cyclopropylmethyl)-3,4-dihydrothieno[2,3-f][1,4]thiazepin-5(2H)-one 1,1-dioxide in step 2. The title compound was isolated. m/z (ESI, +ve)=626.0 [M+H]⁺. ¹H NMR (400 MHz, DMSO-d₆) δ 10.22 (s, 1H), 9.13-9.06 (m, 1H), 8.89 (s, 1H), 7.85 (s, 1H), 7.54-7.46 (m, 2H), 4.35-4.27 (m, 2H), 3.99-3.93 (m, 4H), 3.43-3.37 (m, 4H), 3.06-2.95 (m, 2H), 1.12-1.03 (m, 1H), 0.56-0.49 (m, 2H), 0.36-0.29 (m, 2H).

### Example 154: 7-(2-((7-chloro-2-methyl-1,2,3,4-tetrahydroisoquinolin-6-yl)amino)-5-(trifluoromethyl)pyrimidin-4-yl)-4-(cyclopropylmethyl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 7-(2-((7-chloro-1,2,3,4-tetrahydroisoquinolin-6-yl)amino)-5-(trifluoromethyl)pyrimidin-4-yl)-4-(cyclopropylmethyl)-3,4-dihydrothieno[2,3-f][1,4]thiazepin-5(2H)-one 1,1-dioxide. The title compound was isolated in 52% yield. m/z (ESI, +ve)=612.0 [M+H]⁺. ¹H NMR (400 MHz, DMSO-d₆) δ 10.24 (s, 1H), 10.09 (brs, 1H), 8.90 (brs, 1H), 7.85 (s, 1H), 7.56-7.51 (m, 1H), 7.49 (s, 1H), 4.62-4.44 (m, 1H), 4.40-4.25 (m, 1H), 4.00-3.91 (m, 4H), 3.76-3.63 (m, 1H), 3.51-3.44 (m, 1H), 3.16-3.05 (m, 2H), 2.96 (s, 3H), 2.55-2.52 (m, 2H), 1.13-1.01 (m, 1H), 0.57-0.49 (m, 2H), 0.36-0.27 (m, 2H).

### Example 155: 7-(2-((7-chloro-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide

### Step 1: 4-cyclopropyl-3,4-dihydrothieno[2,3-f][1,4]thiazepin-5(2H)-one 1,1-dioxide

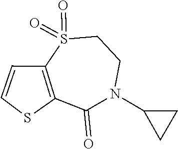 [SMILES: O=C1c2sccc2S(=O)(=O)CCN1C1CC1]

A mixture of 3,4-dihydrothieno[2,3-f][1,4]thiazepin-5(2H)-one 1,1-dioxide (2.8 mmol), cyclopropylboronic acid (8.3 mmol), copper diacetate (2.8 mmol), pyridine (13.8 mmol) and 4A molecular sieves (1.8 g) in 1,2-dichloroethane (6 mL) was stirred at 80° C. for 12 hours under air. Additional copper acetate was added (2.8 mmol) and stirring continued at 80° C. for another 12 hours. The mixture was cooled down, filtered, and concentrated to afford a residue that was purified by preparative TLC (66% ethyl acetate in hexanes). The title compound was isolated in 17% yield. MS (ESI) m/z: 258.1 [M+H]⁺.

### Step 2: 7-bromo-4-cyclopropyl-3,4-dihydrothieno[2,3-f][1,4]thiazepin-5(2H)-one 1,1-dioxide

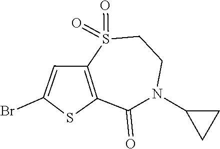 [SMILES: O=C1c2sc(Br)cc2S(=O)(=O)CCN1C1CC1]

A solution of 4-cyclopropyl-3,4-dihydrothieno[2,3-f][1,4]thiazepin-5(2H)-one 1,1-dioxide (0.85 mmol) in acetic acid (2 mL) was treated with N-bromosuccinimide (1.71 mmol) and sulfuric acid (8.6 mmol). The mixture was stirred at 60° C. for 12 hours, cooled down to room temperature and poured over ice-water. The solution was extracted with ethyl acetate three times and the combined organic layers were dried over anhydrous sodium sulfate, filtered, and concentrated under reduced pressure to afford a residue that was purified by silica gel chromatography (70% ethyl acetate in hexanes). The title compound was isolated in 84% yield. MS (ESI) m/z: 336.0 [M+H]⁺.

Step 3: 7-(2-((7-chloro-2-(2,2,2-trifluoroacetyl)-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide

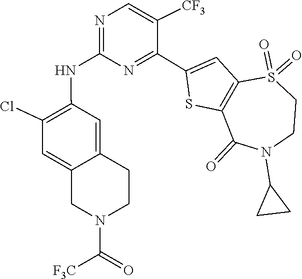 [SMILES: O=C1c2sc(-c3nc(Nc4cc5c(cc4Cl)CN(C(=O)C(F)(F)F)CC5)ncc3C(F)(F)F)cc2S(=O)(=O)CCN1C1CC1]

A solution of 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one (0.03 mmol) and 7-bromo-4-cyclopropyl-3,4-dihydrothieno[2,3-f][1,4]thiazepin-5(2H)-one 1,1-dioxide (0.03 mmol) in dioxane (1 mL) was treated with copper (I) iodide (0.03 mmol) and tetrakis[triphenylphosphine]palladium (0) (0.003 mmol). The mixture was heated at 120° C. for 12 hours, cooled down to room temperature and concentrated under reduced pressure to afford a residue that was purified by preparative TLC (66% ethyl acetate in hexanes). The title compound was isolated in 46% yield. MS (ESI) m/z: 680.3[M+H]⁺.

### Step 4: 7-(2-((7-chloro-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide

A mixture of 7-(2-((7-chloro-2-(2,2,2-trifluoroacetyl)-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide (0.01 mmol) and potassium carbonate (0.09 mmol) in acetonitrile (2 mL) and water (0.5 mL) was stirred at room temperature for 12 hours. After evaporation of volatiles, the crude product was purified by preparative HPLC to afford the title compound in 91% yield. MS (ESI) m/z: 584.2 [M+H]⁺. ¹H NMR (400 MHz, methanol-d₄) δ 8.80 (s, 1H), 8.54 (s, 1H), 8.00 (s, 1H), 7.80 (s, 1H), 7.31 (s, 1H), 4.15 (s, 2H), 4.02-3.95 (m, 2H), 3.80-3.75 (m, 2H), 3.34-3.32 (m, 2H), 3.03 (t, J=6.0 Hz, 2H), 2.94-2.86 (m, 1H), 0.93-0.89 (m, 4H).

### Example 156: 7-(2-((7-chloro-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 155, steps 3-4, where 7-bromo-4-cyclopropyl-3,4-dihydrothieno[2,3-f][1,4]thiazepin-5(2H)-one 1,1-dioxide was replaced with 7-bromo-4-methyl-3,4-dihydrothieno[2,3-f][1,4]thiazepin-5(2H)-one 1,1-dioxide in step 3. The title compound was isolated in 68% yield. MS (ESI) m/z: 557.9 [M+H]⁺. ¹H NMR (400 MHz, DMSO-d₆) δ 10.20 (s, 1H), 9.10-9.06 (m, 1H), 8.88 (s, 1H), 7.84 (s, 1H), 7.51 (s, 2H), 4.34-4.27 (m, 2H), 3.99-3.94 (m, 2H), 3.92-3.87 (m, 2H), 3.44-3.39 (m, 2H), 3.10-3.06 (m, 3H), 3.03-2.97 (m, 2H).

### Example 157: 7-(2-((6-fluoroisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 2,2,2-trifluoro-1-(5-fluoro-6-((5-(trifluoromethyl)-4-(trimethylstannyl)pyrimidin-2-yl)amino)isoindolin-2-yl)ethan-1-one and 7-bromo-4-methyl-3,4-dihydrothieno[2,3-f][1,4]thiazepin-5(2H)-one 1,1-dioxide in step 3. The title compound was isolated. MS (ESI) m/z: 528.1 [M+H]⁺. ¹H NMR (400 MHz, methanol-d₄) δ 8.84 (s, 1H), 8.06 (d, J=7.2 Hz, 1H), 8.03 (s, 1H), 7.34 (d, J=10.4 Hz, 1H), 4.65 (d, J=6.4 Hz, 4H), 3.96 (d, J=5.6 Hz, 2H), 3.89-3.88 (m, 2H), 3.20 (s, 3H).

### Example 158: 7-(2-((7-chloro-1,2,3,4-tetrahydroisoquinolin-6-yl)amino)-5-(trifluoromethyl)pyrimidin-4-yl)-4-cyclobutyl-3,4-dihydrothieno[2,3-f][1,4]thiazepin-5(2H)-one 1,1-dioxide

### Step 1: 7-bromo-4-cyclobutyl-3,4-dihydrothieno[2,3-f][1,4]thiazepin-5(2H)-one 1,1-dioxide

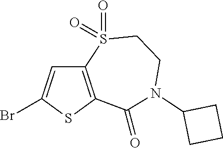 [SMILES: O=C1c2sc(Br)cc2S(=O)(=O)CCN1C1CCC1]

A −20° C. solution of 7-bromo-3,4-dihydrothieno[2,3-f][1,4]thiazepin-5(2H)-one 1,1-dioxide (0.34 mmol) in DMF (2.5 mL) was treated with 60% NaH (0.51 mmol). After 30 minutes, bromocyclobutane (1.0 mmol) was added and the reaction as stirred at 80° C. for 12 hours. The reaction mixture was diluted with water, extracted with ethyl acetate three times and the combined organic layers were dried over anhydrous sodium sulfate, filtered, and concentrated under reduced pressure to afford a residue that was purified by silica gel chromatography (50% ethyl acetate in hexanes). The title compound was isolated in 15% yield. MS (ESI) m/z: 350.0 [M+H]⁺.

### Steps 2-3: 7-(2-((7-chloro-1,2,3,4-tetrahydroisoquinolin-6-yl)amino)-5-(trifluoromethyl)pyrimidin-4-yl)-4-cyclobutyl-3,4-dihydrothieno[2,3-f][1,4]thiazepin-5(2H)-one 1,1-dioxide

The title compound was prepared analogously to Example 155, steps 3-4, where 7-bromo-4-cyclopropyl-3,4-dihydrothieno[2,3-f][1,4]thiazepin-5(2H)-one 1,1-dioxide was replaced with 7-bromo-4-cyclobutyl-3,4-dihydrothieno[2,3-f][1,4]thiazepin-5(2H)-one 1,1-dioxide in step 3. The title compound was isolated in 54% yield. MS (ESI) m/z: 598.0 [M+H]⁺. ¹H NMR (400 MHz, DMSO-d₆) δ 10.22 (s, 1H), 9.04-9.00 (m, 1H), 8.92-8.84 (m, 1H), 7.86-7.81 (m, 1H), 7.53-7.46 (m, 2H), 4.73-4.60 (m, 1H), 4.34-4.25 (m, 2H), 3.98-3.84 (m, 4H), 3.44-3.41 (m, 2H), 3.04-2.96 (m, 2H), 2.27-2.18 (m, 2H), 2.17-2.09 (m, 2H), 1.73-1.60 (m, 2H).

### Example 159: 7-(2-((6-chloro-1,2,3,4-tetrahydroisoquinolin-7-yl)amino)-5-(trifluoromethyl)pyrimidin-4-yl)-4-methyl-3,4-dihydrothieno[2,3-f][1,4]thiazepin-5(2H)-one 1,1-dioxide

### Step 1: 1-(6-chloro-7-((4-chloro-5-(trifluoromethyl)pyrimidin-2-yl)amino)-3,4-dihydroisoquinolin-2(1H)-yl)-2,2,2-trifluoroethan-1-one

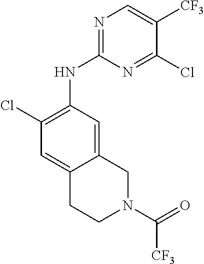 [SMILES: O=C(N1CCc2cc(Cl)c(Nc3ncc(C(F)(F)F)c(Cl)n3)cc2C1)C(F)(F)F]

The title compound was prepared analogously to Example 1, step 5 where 1-(6-amino-7-chloro-1,2,3,4-tetrahydroisoquinolin-2-yl)-2,2,2-trifluoroethan-1-one was replaced with 1-(7-amino-6-chloro-3,4-dihydroisoquinolin-2(1H)-yl)-2,2,2-trifluoroethan-1-one. The title compound was isolated in 32% yield. MS (ESI) m z: 459.0 [M+H]⁺.

### Step 2: 1-(6-chloro-7-((5-(trifluoromethyl)-4-(trimethylstannyl)pyrimidin-2-yl)amino)-3,4-dihydroisoquinolin-2(1H)-yl)-2,2,2-trifluoroethan-1-one

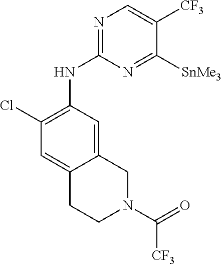

The title compound was prepared analogously to Example 4, step 1 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with 1-(6-chloro-7-((4-chloro-5-(trifluoromethyl)pyrimidin-2-yl)amino)-3,4-dihydroisoquinolin-2(1H)-yl)-2,2,2-trifluoroethan-1-one. The title compound was isolated in 26% yield after purification by neutral alumina. MS (ESI) m/z: 589.0 [M+H]⁺.

### Step 3-4: 7-(2-((6-chloro-1,2,3,4-tetrahydroisoquinolin-7-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(6-chloro-7-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-methyl-3,4-dihydrothieno[2,3-f][1,4]thiazepin-5(2H)-one 1,1-dioxide in step 3. The title compound was isolated in 58% yield. MS (ESI) m/z: 694.0 [M+H]⁺.

### Example 160: 7-(2-((6-ethylisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide

### Step 1: 4-methyl-7-(trimethylstannyl)-3,4-dihydrothieno[2,3-f][1,4]thiazepin-5(2H)-one 1,1-dioxide

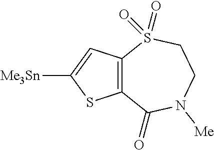

A mixture of 7-bromo-4-methyl-3,4-dihydrothieno[2,3-f][1,4]thiazepin-5(2H)-one 1,1-dioxide (2.9 mmol), hexamethylditin (5.8 mmol) and tetrakis(triphenylphosphine)palladium (0) (0.14 mmol) in dioxane (10 mL) was stirred at 90° C. for 12 hours. The reaction was concentrated under reduced pressure and the crude material purified by silica gel chromatography (0-100% ethyl acetate) to afford the title compound in 35% yield. MS (ESI) m/z: 395.90 [M+H]⁺.

### Steps 2 and 3: 7-(2-((6-ethylisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 155, steps 3-4, where 7-bromo-4-cyclopropyl-3,4-dihydrothieno[2,3-f][1,4]thiazepin-5(2H)-one 1,1-dioxide and 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one were replaced with 1-(5-((4-chloro-5-(trifluoromethyl)pyrimidin-2-yl)amino)-6-ethylisoindolin-2-yl)-2,2,2-trifluoroethan-1-one and 4-methyl-7-(trimethylstannyl)-3,4-dihydrothieno[2,3-f][1,4]thiazepin-5(2H)-one 1,1-dioxide in step 3. The title compound was isolated. MS (ESI) m/z: 536.2 [M+H]⁺. ¹H NMR (400 MHz, DMSO-d₆) δ 9.88 (s, 1H), 8.80 (s, 1H), 7.81 (s, 1H), 7.26 (s, 1H), 7.19 (s, 1H), 4.06 (m, 4H), 3.96 (bs, 2H), 3.89 (bs, 2H), 3.08 (s, 3H), 2.59 (q, J=7.4 Hz, 2H), 1.10 (t, J=7.5 Hz, 3H).

### Example 161: 4-methyl-7-(2-((7-(methylthio)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 4-methyl-7-(trimethylstannyl)-3,4-dihydrothieno[2,3-f][1,4]thiazepin-5(2H)-one 1,1-dioxide and 1-(6-((4-chloro-5-(trifluoromethyl)pyrimidin-2-yl)amino)-7-(methylthio)-3,4-dihydroisoquinolin-2(1H)-yl)-2,2,2-trifluoroethan-1-one in step 3. The title compound was isolated. MS (ESI) m/z: 570.1 [M+H]⁺. ¹H NMR (400 MHz, DMSO-d₆) δ 9.84 (s, 1H), 8.82 (s, 1H), 7.82 (s, 1H), 7.09 (d, J=21.0 Hz, 2H), 3.96 (d, J=5.8 Hz, 2H), 3.93-3.82 (m, 5H), 3.08 (d, J=3.5 Hz, 3H), 2.96 (t, J=5.8 Hz, 2H), 2.70-2.64 (m, 2H), 2.37 (s, 3H).

### Example 162: 7-(2-((7-chloro-2-ethyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-chloro-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide and acetaldehyde. The title compound was isolated in 69% yield. m/z (ESI, +ve)=585.9 [M+H]⁺. ¹H NMR (400 MHz, DMSO-d₆) δ 10.04 (br s, 1H), 8.85 (s, 1H), 7.82 (s, 1H), 7.34 (s, 1H), 7.29 (s, 1H), 3.98-3.94 (m, 2H), 3.90-3.86 (m, 2H), 3.57-3.53 (m, 2H), 3.07 (s, 3H), 2.83-2.78 (m, 2H), 2.66-2.63 (m, 2H), 2.60-2.56 (m, 2H), 1.10 (t, J=7.2 Hz, 3H).

### Example 163: 7-(2-((7-chloro-1,2,3,4-tetrahydroisoquinolin-6-yl)amino)-5-(trifluoromethyl)pyrimidin-4-yl)-2,3-dihydro-5H-thieno[3,2-e][1,4]oxathiepine 1,1-dioxide

### Step 1: 2-((3-bromothiophen-2-yl)methoxy)ethan-1-ol

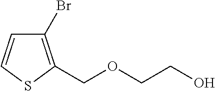 [SMILES: OCCOCc1sccc1Br]

To a solution of ethane-1,2-diol (135 mmol) in dimethylsulfoxide (50 mL) was added potassium tert-butoxide (35 mmol) and 3-bromo-2-(chloromethyl)thiophene (27 mmol). A solution of tetrabutylammonium iodide (2.69 mmol) in dimethylsulfoxide (10 mL) was added and the mixture was stirred at room temperature for 2 hours. The reaction mixture was poured into water and extracted with ethyl acetate twice. The combined organic layers were washed with brine, dried over anhydrous sodium sulfate, filtered, and concentrated under reduced pressure to afford a residue that was purified by silica gel chromatography (17% ethyl acetate in hexanes). The title compound was isolated in 58% yield.

### Step 2: S-(2-((3-bromothiophen-2-yl)methoxy)ethyl) ethanethioate

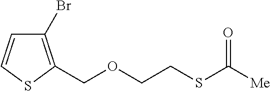 [SMILES: CC(=O)SCCOCc1sccc1Br]

To a 0° C. solution of triphenylphosphine (31 mmol) in THF (20 mL) was added dropwise a solution of diisopropylazodicarboxylate (31 mmol) in THF (8 mL). After 1 hour, a solution of 2-((3-bromothiophen-2-yl)methoxy)ethan-1-ol (16 mmol) in THF (8 mL) was added dropwise at 0° C., followed by ethanethioic S-acid (31 mmol). The reaction was stirred at room temperature for 2 hours and the volatiles were removed under reduced pressure. The crude material was purified by preparative TLC (9% ethyl acetate in hexanes). The title compound was isolated in 39% yield.

### Step 3: 2-((3-Bromothiophen-2-yl)methoxy)ethane-1-thiol

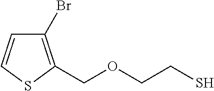 [SMILES: SCCOCc1sccc1Br]

A solution of S-(2-((3-bromothiophen-2-yl)methoxy)ethyl) ethanethioate (6.1 mmol) in methanol (1 mL) was treated with 1M aqueous NaOH (18 mmol). After 1 hour the reaction was concentrated to afford the title compound. This material was used in the next step without further purification. MS (ESI) m/z: 252.2 [M+H]⁺. ¹H NMR (400 MHz, CDCl₃) δ=7.30 (d, J=5.2 Hz, 1H), 6.97 (d, J=5.2 Hz, 1H), 4.69 (s, 2H), 3.67 (t, J=6.4 Hz, 2H), 2.76-2.70 (m, 2H), 1.63 (t, J=8.0 Hz, 1H).

### Step 4: 2,3-dihydro-5H-thieno[3,2-e][1,4]oxathiepine

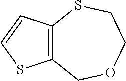

A solution of 2-((3-bromothiophen-2-yl)methoxy)ethane-1-thiol (4.7 mmol), N,N-diisopropylethylamine (14 mmol), tris(dibenzylidenacetone)dipalladium (0.47 mmol) and Xantphos (0.47 mmol) in dioxane (20 mL) was stirred at 120° C. for 8 hours. After evaporation of volatiles under reduced pressure, the residue was purified by silica gel chromatography (9% ethyl acetate in hexanes). The title compound was isolated in 53% yield.

### Step 5: 7-bromo-2,3-dihydro-5H-thieno[3,2-e][1,4]oxathiepine

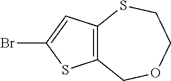 [SMILES: Brc1cc2c(s1)COCCS2]

The title compound was prepared analogously to Example 125, step 4, where 3,4-dihydrothieno[2,3-f][1,4]thiazepin-5(2H)-one 1,1-dioxide was replaced with 2,3-dihydro-5H-thieno[3,2-e][1,4]oxathiepine. The title compound was isolated in 34% yield. ¹H NMR (400 MHz, CDCl₃) δ=6.97 (s, 1H), 4.70 (s, 2H), 4.22-4.20 (m, 2H), 2.82-2.80 (m, 2H).

### Step 6: 7-bromo-2,3-dihydro-5H-thieno[3,2-e][1,4]oxathiepine 1,1-dioxide

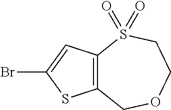 [SMILES: O=S1(=O)CCOCc2sc(Br)cc21]

To a 0° C. solution of 7-bromo-2,3-dihydro-5H-thieno[3,2-e][1,4]oxathiepine (0.80 mmol) in dichloromethane (1 mL) was added meta-chloroperoxybenzoic acid (0.74 mmol). After 20 minutes, the reaction was poured into water and extracted with ethyl acetate twice. The combined organic layers were washed with brine, dried over anhydrous sodium sulfate, filtered, and concentrated under reduced pressure to afford a residue that was purified by preparative TLC (25% ethyl acetate in hexanes). The title compound was isolated in 66% yield.

### Step 7 and 8: 7-(2-((7-chloro-1,2,3,4-tetrahydroisoquinolin-6-yl)amino)-5-(trifluoromethyl)pyrimidin-4-yl)-2,3-dihydro-5H-thieno[3,2-e][1,4]oxathiepine 1,1-dioxide

The title compound was prepared analogously to Example 155, steps 3-4, where 7-bromo-4-cyclopropyl-3,4-dihydrothieno[2,3-f][1,4]thiazepin-5(2H)-one 1,1-dioxide was replaced with 7-bromo-2,3-dihydro-5H-thieno[3,2-e][1,4]oxathiepine 1,1-dioxide in step 3. The title compound was isolated. MS (ESI) m/z: 531.1 [M+H]⁺. ¹H NMR (400 MHz, CDCl₃) δ=8.75 (s, 1H), 8.14 (s, 2H), 7.74 (s, 1H), 7.13 (s, 1H), 5.00 (s, 2H), 4.47-4.44 (m, 2H), 4.03 (s, 2H), 3.45-3.43 (m, 2H), 3.22-3.19 (m, 2H), 2.91-2.88 (m, 2H), 1.36-1.25 (m, 1H).

### Example 164: 7-(2-((6-chloroisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide

### Step 1: 1-(5-chloro-6-((5-(trifluoromethyl)-4-(trimethylstannyl)pyrimidin-2-yl)amino)isoindolin-2-yl)-2,2,2-trifluoroethan-1-one

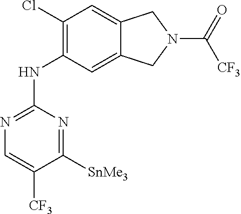

The title compound was prepared analogously to Example 80, step 3 where 1-(5-((4-chloro-5-(trifluoromethyl)pyrimidin-2-yl)amino)-6-fluoroisoindolin-2-yl)-2,2,2-trifluoroethan-1-one was replaced with 1-(5-chloro-6-((4-chloro-5-(trifluoromethyl)pyrimidin-2-yl)amino)isoindolin-2-yl)-2,2,2-trifluoroethan-1-one. The title compound was isolated in 32% yield. MS (ESI) m/z: 574.9 [M+H]⁺.

### Step 2: 7-(2-((6-chloroisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(5-chloro-6-((5-(trifluoromethyl)-4-(trimethylstannyl)pyrimidin-2-yl)amino)isoindolin-2-yl)-2,2,2-trifluoroethan-1-one and 7-bromo-4-methyl-3,4-dihydrothieno[2,3-f][1,4]thiazepin-5(2H)-one 1,1-dioxide in step 3. The title compound was isolated. MS (ESI) m/z: 544.1 [M+H]⁺. ¹H NMR (400 MHz, CDCl₃) δ=8.79 (s, 1H), 8.25 (s, 1H), 8.11 (s, 1H), 7.35 (s, 1H), 4.34 (d, J=1.1 Hz, 2H), 4.26 (s, 2H), 3.96-3.90 (m, 2H), 3.75 (s, 2H), 3.26 (s, 3H), 1.30-1.24 (m, 1H).

### Example 165: 7-(2-((3-methyl-1-(piperidin-4-yl)-1H-pyrazol-4-yl)amino)-5-(trifluoromethyl)pyrimidin-4-yl)-3,4-dihydrothieno[2,3-f][1,4]thiazepin-5(2H)-one 1,1-dioxide

### Step 1: tert-butyl 4-(4-((4-chloro-5-(trifluoromethyl)pyrimidin-2-yl)amino)-3-methyl-1H-pyrazol-1-yl)piperidine-1-carboxylate

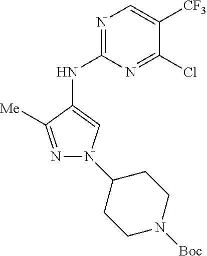 [SMILES: Cc1nn(C2CCN(C(=O)OC(C)(C)C)CC2)cc1Nc1ncc(C(F)(F)F)c(Cl)n1]

The title compound was prepared analogously to Example 123 (steps 1-3), where 1-Boc-3-hydroxyazetidine was replaced with tert-butyl 4-hydroxypiperidine-1-carboxylate in step 1. The title compound was isolated. MS (ESI) m/z: 405.1 [M+H]⁺. ¹H NMR (400 MHz, DMSO-d₆) δ 9.11 (s, 1H), 8.61-8.43 (m, 1H), 7.79 (s, 1H), 7.43 (s, 1H), 4.47-4.19 (m, 1H), 4.04 (d, J=10.5 Hz, 2H), 2.90 (s, 2H), 2.13 (s, 1H), 2.00 (s, 3H), 1.94-1.65 (m, 3H), 1.50-1.32 (m, 9H).

### Step 2: tert-butyl 4-(3-methyl-4-((5-(trifluoromethyl)-4-(trimethylstannyl)pyrimidin-2-yl)amino)-1H-pyrazol-1-yl)piperidine-1-carboxylate

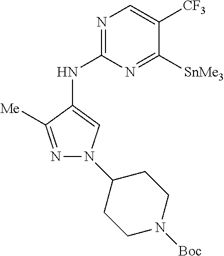

The title compound was prepared analogously to Example 160, step 1, where 7-bromo-4-methyl-3,4-dihydrothieno[2,3-f][1,4]thiazepin-5(2H)-one 1,1-dioxide was replaced with tert-butyl 4-(4-((4-chloro-5-(trifluoromethyl)pyrimidin-2-yl)amino)-3-methyl-1H-pyrazol-1-yl)piperidine-1-carboxylate. The title compound was isolated in 42% yield. MS (ESI) m/z: 591.2 [M+H]⁺.

### Step 3: 7-(2-((3-methyl-1-(piperidin-4-yl)-1H-pyrazol-4-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 149, steps 4-5, where tert-butyl 4-(4-chloro-3-((5-(trifluoromethyl)-4-(trimethylstannyl)pyrimidin-2-yl)amino)phenyl)piperidine-1-carboxylate and 7-bromo-3,4-dihydrothieno[2,3-f][1,4]thiazepin-5(2H)-one 1,1-dioxide were replaced with tert-butyl 4-(3-methyl-4-((5-(trifluoromethyl)-4-(trimethylstannyl)pyrimidin-2-yl)amino)-1H-pyrazol-1-yl)piperidine-1-carboxylate and 7-bromo-4-methyl-3,4-dihydrothieno[2,3-f][1,4]thiazepin-5(2H)-one 1,1-dioxide in step 4. The title compound was isolated. MS (ESI) m/z: 542.2[M+H]⁺. ¹H NMR (400 MHz, methanol-d₄) δ 8.58 (d, J=0.8 Hz, 1H), 8.10 (s, 1H), 7.91 (s, 1H), 4.27 (m, 1H), 3.78 (s, 4H), 3.23 (m, 2H), 2.86-2.75 (m, 2H), 2.18 (s, 5H), 2.02-1.91 (m, 2H).

### Example 166: 4-methyl-7-(2-((3-methyl-1-(piperidin-4-yl)-1H-pyrazol-4-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 149, steps 4-5, where tert-butyl 4-(4-chloro-3-((5-(trifluoromethyl)-4-(trimethylstannyl)pyrimidin-2-yl)amino)phenyl)piperidine-1-carboxylate was replaced with tert-butyl 4-(3-methyl-4-((5-(trifluoromethyl)-4-(trimethylstannyl)pyrimidin-2-yl)amino)-1H-pyrazol-1-yl)piperidine-1-carboxylate and 7-bromo-4-methyl-3,4-dihydrothieno[2,3-f][1,4]thiazepin-5(2H)-one 1,1-dioxide in step 4. The title compound was isolated. MS (ESI) m/z: 556.0[M+H]⁺. ¹H NMR (400 MHz, methanol-d₄) δ 8.56 (s, 1H), 8.01 (s, 1H), 7.87 (s, 1H), 4.34-4.26 m, 1H), 3.98-3.89 (m, 2H), 3.84-3.76 (m, 2H), 3.16 (s, 3H), 3.03-2.71 (m, 2H), 2.21 (d, J=11.6 Hz, 2H), 2.16 (s, 3H), 2.06-1.96 (m, 2H), 1.27 (s, 2H).

### Example 167: 7-(2-((7-chloro-2-methyl-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 7-(2-((7-chloro-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide. The title compound was isolated in 83% yield. m/z (ESI, +ve)=598.2 [M+H]⁺. ¹H NMR (400 MHz, methanol-d₄) δ 8.78 (s, 1H), 8.00 (s, 1H), 7.72 (s, 1H), 7.25 (s, 1H), 3.97 (t, J=6.0 Hz, 2H), 3.80-3.75 (m, 2H), 3.62 (s, 2H), 3.01 (t, J=6.0 Hz, 2H), 2.94-2.87 (m, 1H), 2.81-2.75 (m, 2H), 2.47 (s, 3H), 0.93-0.89 (m, 4H).

### Example 168: 7-(2-((7-chloro-2-methyl-1,2,3,4-tetrahydroisoquinolin-6-yl)amino)-5-(trifluoromethyl)pyrimidin-4-yl)-4-cyclobutyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 7-(2-((7-chloro-1,2,3,4-tetrahydroisoquinolin-6-yl)amino)-5-(trifluoromethyl)pyrimidin-4-yl)-4-cyclobutyl-3,4-dihydrothieno[2,3-f][1,4]thiazepin-5(2H)-one 1,1-dioxide. The title compound was isolated in 28% yield. m/z (ESI, +ve)=612.0 [M+H]⁺. ¹H NMR (400 MHz, DMSO-d₆) δ 10.06 (s, 1H), 8.87 (s, 1H), 7.83 (s, 1H), 7.34 (s, 1H), 7.28 (s, 1H), 4.72-4.63 (m, 1H), 3.94-3.89 (m, 2H), 3.88-3.81 (m, 2H), 3.54-3.49 (m, 2H), 2.87-2.79 (m, 2H), 2.65-2.58 (m, 2H), 2.39-2.34 (m, 3H), 2.24-2.18 (m, 2H), 2.17-2.12 (m, 2H), 1.71-1.63 (m, 2H).

### Example 169: 7-(2-((7-chloro-2-methyl-1,2,3,4-tetrahydroisoquinolin-6-yl)amino)-5-(trifluoromethyl)pyrimidin-4-yl)-4-cyclobutyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 7-(2-((7-chloro-1,2,3,4-tetrahydroisoquinolin-6-yl)amino)-5-(trifluoromethyl)pyrimidin-4-yl)-4-cyclobutyl-3,4-dihydrothieno[2,3-f][1,4]thiazepin-5(2H)-one 1,1-dioxide. The title compound was isolated in 50% yield. m/z (ESI, +ve)=572.1 [M+H]⁺. ¹H NMR (400 MHz, DMSO-d₆) δ 10.06 (br s, 1H), 8.85 (s, 1H), 7.82 (br s, 1H), 7.32 (s, 1H), 7.31-7.27 (m, 1H), 3.99-3.94 (m, 2H), 3.91-3.85 (m, 2H), 3.49-3.46 (m, 2H), 3.08-3.06 (m, 3H), 2.87-2.81 (m, 2H), 2.61-2.59 (m, 2H), 2.34 (s, 3H).

### Example 170: 7-(2-((4-cyclopropylisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide

### Step 1: 1-(5-amino-4-bromoisoindolin-2-yl)-2,2,2-trifluoroethan-1-one

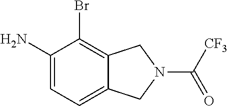 [SMILES: Nc1ccc2c(c1Br)CN(C(=O)C(F)(F)F)C2]

To a 0° C. solution 1-(5-aminoisoindolin-2-yl)-2,2,2-trifluoroethan-1-one (27 mmol) in acetonitrile (100 mL) was added N-bromosuccinimide (27 mmol) and the reaction mixture was stirred for 2 hours. The mixture was concentrated under vacuum and the residue was purified by silica gel chromatography (5-10% ethyl acetate in hexanes) to afford the title compound in 50% yield. MS (ESI) m/z: 308.9 [M+H]⁺. ¹H NMR (400 MHz, DMSO-d₆) ¹H NMR (400 MHz, DMSO-d₆) δ 7.38 (d, J=8.8 Hz, 1H), 7.19-6.96 (m, 1H), 6.85-6.66 (m, 1H), 5.41 (d, J=13.0 Hz, 2H), 5.02-4.81 (m, 2H), 4.80-4.56 (m, 2H).

### Step 2: 1-(5-amino-4-cyclopropylisoindolin-2-yl)-2,2,2-trifluoroethan-1-one

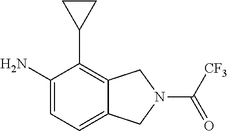 [SMILES: Nc1ccc2c(c1C1CC1)CN(C(=O)C(F)(F)F)C2]

The title compound was prepared analogously to Example 9, step 1, where 1-(7-chloro-6-nitro-1,2,3,4-tetrahydroisoquinolin-2-yl)-2,2,2-trifluoroethan-1-one and ethylboronic acid were replaced with 1-(5-amino-4-bromoisoindolin-2-yl)-2,2,2-trifluoroethan-1-one, and potassium cyclopropyltrifluoroborate. The title compound was isolated in 19% yield. MS (ESI) m/z: 271.1 [M+H]⁺.

### Step 3: 1-(5-((4-chloro-5-(trifluoromethyl)pyrimidin-2-yl)amino)-4-cyclopropylisoindolin-2-yl)-2,2,2-trifluoroethan-1-one

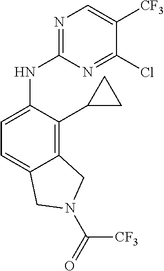 [SMILES: O=C(N1Cc2ccc(Nc3ncc(C(F)(F)F)c(Cl)n3)c(C3CC3)c2C1)C(F)(F)F]

The title compound was prepared analogously to Example 1, step 5 where 1-(6-amino-7-chloro-1,2,3,4-tetrahydroisoquinolin-2-yl)-2,2,2-trifluoroethan-1-one was replaced with 1-(5-amino-4-cyclopropylisoindolin-2-yl)-2,2,2-trifluoroethan-1-one. The title compound was isolated in 50% yield. MS (ESI) m/z: 451.0[M+H]⁺. ¹H NMR (400 MHz, CDCl₃) δ 8.93-8.55 (m, 1H), 8.11-8.07 (m, 1H), 7.24-7.09 (m, 1H), 5.04-4.98 (m, 2H), 4.91-4.85 (m, 2H), 1.80-1.72 (m, 1H), 1.16-1.07 (m, 2H), 0.70-0.61 (m, 2H).

### Step 4: 1-(4-cyclopropyl-5-((5-(trifluoromethyl)-4-(trimethylstannyl)pyrimidin-2-yl)amino)isoindolin-2-yl)-2,2,2-trifluoroethan-1-one

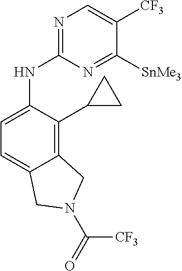

The title compound was prepared analogously to Example 80, step 3 where 1-(5-((4-chloro-5-(trifluoromethyl)pyrimidin-2-yl)amino)-6-fluoroisoindolin-2-yl)-2,2,2-trifluoroethan-1-one was replaced with 1-(5-((4-chloro-5-(trifluoromethyl)pyrimidin-2-yl)amino)-4-cyclopropylisoindolin-2-yl)-2,2,2-trifluoroethan-1-one. The title compound was isolated in 24% yield. MS (ESI) m/z: 580.9[M+H]⁺.

### Step 5: 7-(2-((4-cyclopropylisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(4-cyclopropyl-5-((5-(trifluoromethyl)-4-(trimethylstannyl)pyrimidin-2-yl)amino)isoindolin-2-yl)-2,2,2-trifluoroethan-1-one and 7-bromo-4-methyl-3,4-dihydrothieno[2,3-f][1,4]thiazepin-5(2H)-one 1,1-dioxide in step 3. The title compound was isolated. MS (ESI) m/z: 550.2 [M+H]⁺. ¹H NMR (400 MHz, methanol-d₄) δ 8.73 (s, 1H), 7.99 (s, 1H), 7.49 (d, J=8.0 Hz, 1H), 7.21 (d, J=8.0 Hz, 1H), 4.36 (s, 2H), 4.23 (s, 2H), 3.95-3.92 (m, 2H), 3.86-3.83 (m, 2H), 3.18 (s, 3H), 1.83-1.76 (m, 1H), 0.91-0.86 (m, 2H), 0.55-0.51 (m, 2H).

### Example 171: 4-methyl-7-(2-((2-methyl-7-(methylthio)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 4-methyl-7-(2-((7-(methylthio)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide. The title compound was isolated in 16% yield. MS (ESI) m/z=584.0 [M+H]⁺. ¹H NMR (300 MHz, DMSO-d₆) δ 9.84 (s, 1H), 8.82 (s, 1H), 7.81 (s, 1H), 7.16 (s, 1H), 7.09 (s, 1H), 3.95 (d, J=5.5 Hz, 2H), 3.89 (s, 2H), 3.51 (s, 2H), 3.07 (s, 3H), 2.80 (s, 2H), 2.59 (t, J=6.0 Hz, 2H), 2.36 (d, J=5.1 Hz, 5H).

### Example 172: 7-(2-((1-(2-(dimethylamino)ethyl)-3-methyl-1H-pyrazol-4-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 123, where 1-Boc-3-hydroxyazetidine was replaced with tert-butyl (2-hydroxyethyl)(methyl)carbamate in step 1 and trimethyl(4-(methylsulfonyl)thiophen-2-yl)stannane with 4-methyl-7-(trimethylstannyl)-3,4-dihydrothieno[2,3-f][1,4]thiazepin-5(2H)-one 1,1-dioxide in step 4. The title compound was isolated.

MS (ESI) m/z=528.3 [M+H]⁺. ¹H NMR (400 MHz, DMSO-d₆) δ 9.80 (d, J=69.8 Hz, 1H), 8.82 (d, J=12.9 Hz, 1H), 8.00-7.75 (m, 2H), 4.07 (t, J=6.4 Hz, 2H), 4.02-3.95 (m, 2H), 3.89 (d, J=6.1 Hz, 2H), 3.08 (d, J=6.6 Hz, 3H), 2.85 (q, J=6.5 Hz, 2H), 2.28 (s, 3H), 2.13 (d, J=6.8 Hz, 3H).

### Example 173: 7-(2-((7-cyclopropyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

### Step 1: 1-(7-bromo-3,4-dihydroisoquinolin-2(1H)-yl)-2,2,2-trifluoroethan-1-one

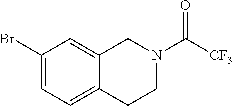 [SMILES: O=C(N1CCc2ccc(Br)cc2C1)C(F)(F)F]

To a solution of 7-bromo-1,2,3,4-tetrahydroisoquinoline (60 mmol) in dichloromethane (150 mL) was added pyridine (181 mmol), followed by dimethylaminopyridine (3.0 mmol) and trifluoroacetic anhydride (72 mmol). The mixture was stirred for 2 hours, quenched with water, and extracted with dichloromethane three times. The combined organic layers were washed with brine, dried with anhydrous sodium sulfate, filtered, and concentrated to afford a residue that was purified by silica gel chromatography (0-12% ethyl acetate in hexanes). The title compound was isolated in 97% yield.

MS (ESI) m/z: 309.9 [M+H]⁺. ¹H NMR (400 MHz, DMSO-d₆) δ=7.65-7.21 (m, 2H), 7.21-6.98 (m, 1H), 4.79-4.69 (m, 2H), 3.85-3.74 (m, 2H), 2.86 (q, J=5.6 Hz, 2H).

### Step 2: 1-(7-bromo-6-nitro-3,4-dihydroisoquinolin-2(1H)-yl)-2,2,2-trifluoroethan-1-one

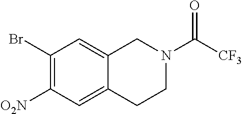 [SMILES: O=C(N1CCc2cc([N+](=O)[O-])c(Br)cc2C1)C(F)(F)F]

The title compound was prepared analogously to Example 1, step 3, where 1-(7-chloro-1,2,3,4-tetrahydroisoquinolin-2-yl)-2,2,2-trifluoroethan-1-one was replaced with 1-(7-bromo-3,4-dihydroisoquinolin-2(1H)-yl)-2,2,2-trifluoroethan-1-one. The title compound was isolated in 80% yield. MS (ESI) m/z: 353.09 [M+H]⁺. ¹H NMR (400 MHz, DMSO-d₆) δ 7.92 (d, J=17.6 Hz, 2H), 4.92-4.78 (m, 2H), 3.86-3.78 (m, 2H), 3.01-2.92 (m, 2H).

### Step 3: 1-(7-cyclopropyl-6-nitro-3,4-dihydroisoquinolin-2(1H)-yl)-2,2,2-trifluoroethan-1-one

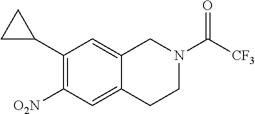 [SMILES: O=C(N1CCc2cc([N+](=O)[O-])c(C3CC3)cc2C1)C(F)(F)F]

A mixture of 1-(7-bromo-6-nitro-3,4-dihydro-1H-isoquinolin-2-yl)-2,2,2-trifluoro-ethanone (8.10 mmol), cyclopropylboronic acid (32 mmol), tricyclohexylphosphane (1.61 mmol), diacetoxypalladium (0.81 mmol) and potassium phosphate (32.3 mmol) in toluene (30 mL) was stirred at 110° C. for 12 hours. The mixture was filtered and concentrated. The residue was purified by preparative TLC (25% ethyl acetate in hexanes) to afford the title compound in 20% yield. ¹H NMR (400 MHz, CDCl₃) δ 7.71-7.66 (m, 1H), 6.98-6.92 (m, 1H), 4.86-4.73 (m, 2H), 3.89 (td, J=6.0, 18.0 Hz, 2H), 3.06-2.95 (m, 2H), 2.44-2.33 (m, 1H), 1.11-1.02 (m, 2H), 0.73-0.64 (m, 2H).

### Step 4: 1-(6-amino-7-cyclopropyl-3,4-dihydroisoquinolin-2(1H)-yl)-2,2,2-trifluoroethan-1-one

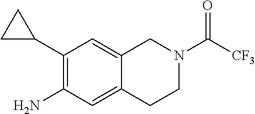 [SMILES: Nc1cc2c(cc1C1CC1)CN(C(=O)C(F)(F)F)CC2]

To a solution of 1-(7-cyclopropyl-6-nitro-3,4-dihydro-1H-isoquinolin-2-yl)-2,2,2-trifluoro-ethanone (1.91 mmol) in a mixture of ethanol (5.6 mL) and water (1.4 mL) was added iron powder (9.55 mmol) and ammonium chloride (9.55 mmol). The mixture was stirred at 70° C. for 2 hours, cooled down to room temperature, filtered and concentrated. The residue was purified by silica gel chromatography (0-32% ethyl acetate in hexanes) to afford the title compound in 65% yield. MS (ESI) m/z: 285.0[M+H]⁺.

### Step 5: 1-(6-((4-chloro-5-(trifluoromethyl)pyrimidin-2-yl)amino)-7-cyclopropyl-3,4-dihydroisoquinolin-2(1H)-yl)-2,2,2-trifluoroethan-1-one

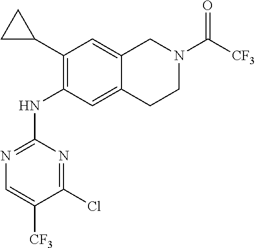 [SMILES: O=C(N1CCc2cc(Nc3ncc(C(F)(F)F)c(Cl)n3)c(C3CC3)cc2C1)C(F)(F)F]

A mixture of 1-(6-amino-7-cyclopropyl-3,4-dihydro-1H-isoquinolin-2-yl)-2,2,2-trifluoro-ethanone (1.23 mmol) and 2,4-dichloro-5-(trifluoromethyl)pyrimidine (6.16 mmol) was stirred at 70° C. for 12 hours. The reaction was cooled down to room temperature, filtered and concentrated. The residue was purified by silica gel chromatography (0-32% ethyl acetate in hexanes) to afford the title compound in 52% yield. MS (ESI) m/z: 465.1[M+H]⁺.

### Step 6: 1-(7-cyclopropyl-6-((5-(trifluoromethyl)-4-(trimethylstannyl)pyrimidin-2-yl)amino)-3,4-dihydroisoquinolin-2(1H)-yl)-2,2,2-trifluoroethan-1-one

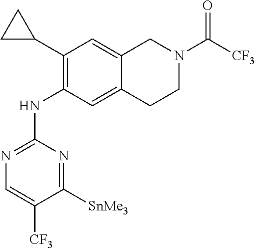

The title compound was prepared analogously to Example 4, step 1 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with 1-(6-((4-chloro-5-(trifluoromethyl)pyrimidin-2-yl)amino)-7-cyclopropyl-3,4-dihydroisoquinolin-2(1H)-yl)-2,2,2-trifluoroethan-1-one. The title compound was isolated in 16% yield. MS (ESI) m/z: 595.1 [M+H]⁺.

### Steps 7-8: 7-(2-((7-cyclopropyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 4-methyl-7-(trimethylstannyl)-3,4-dihydrothieno[2,3-f][1,4]thiazepin-5(2H)-one 1,1-dioxide and 1-(6-((4-chloro-5-(trifluoromethyl)pyrimidin-2-yl)amino)-7-cyclopropyl-3,4-dihydroisoquinolin-2(1H)-yl)-2,2,2-trifluoroethan-1-one in step 3. The title compound was isolated. MS (ESI) m/z: 564.1 [M+H]⁺. ¹H NMR (300 MHz, DMSO-d₆) δ 0.55 (m, 2H), 0.79 (m, 2H), 1.92 (m, 1H), 2.64 (t, J=5.8 Hz, 2H), 2.92 (t, J=5.8 Hz, 2H), 3.07 (s, 3H), 3.79 (s, 2H), 3.89 (m, 2H), 3.95 (m, 2H), 6.64 (s, 1H), 7.10 (s, 1H), 7.81 (s, 1H), 8.80 (s, 1H), 9.84 (s, 1H).

### Example 174: 7-(2-((2-ethyl-4-(piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

### Step 1: tert-butyl 4-(4-((4-chloro-5-(trifluoromethyl)pyrimidin-2-yl)amino)-3-ethylphenyl)piperazine-1-carboxylate

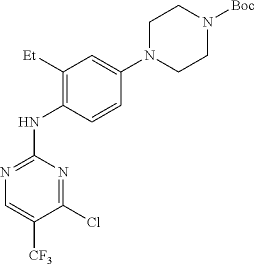 [SMILES: CCc1cc(N2CCN(C(=O)OC(C)(C)C)CC2)ccc1Nc1ncc(C(F)(F)F)c(Cl)n1]

The title compound was prepared analogously to Example 121, step 3, where tert-butyl 3-(3-amino-4-chlorophenyl)azetidine-1-carboxylate was replaced with tert-butyl 4-(4-amino-3-ethylphenyl)piperazine-1-carboxylate. The title compound was isolated. MS (ESI) m/z: 485.9 [M+H]⁺

### Step 2: tert-butyl 4-(3-ethyl-4-((4-(4-methyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)piperazine-1-carboxylate

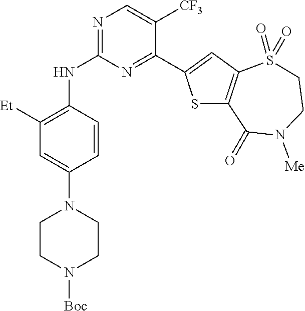 [SMILES: CCc1cc(N2CCN(C(=O)OC(C)(C)C)CC2)ccc1Nc1ncc(C(F)(F)F)c(-c2cc3c(s2)C(=O)N(C)CCS3(=O)=O)n1]

The title compound was prepared analogously to Example 155, step 3, where 7-bromo-4-cyclopropyl-3,4-dihydrothieno[2,3-f][1,4]thiazepin-5(2H)-one 1,1-dioxide and 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one were replaced with tert-butyl 4-(4-((4-chloro-5-(trifluoromethyl)pyrimidin-2-yl)amino)-3-ethylphenyl)piperazine-1-carboxylate and 4-methyl-7-(trimethylstannyl)-3,4-dihydrothieno[2,3-f][1,4]thiazepin-5(2H)-one 1,1-dioxide in step 3. The title compound was isolated in 83% yield. MS (ESI) m/z: 681.2 [M+H]⁺.

### Step 3: 7-(2-((2-ethyl-4-(piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 5, step 2 where tert-butyl 3-(6-((4-(4-carbamoylthiophen-2-yl)-5-(trifluoromethyl)pyrimidin-2-yl)amino)-7-chloro-3,4-dihydroisoquinolin-2(1H)-yl)azetidine-1-carboxylate was replaced with tert-butyl 4-(3-ethyl-4-((4-(4-methyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)piperazine-1-carboxylate. The title compound was isolated in 98% yield. MS (ESI) m/z: 581.0 [M+H]⁺. ¹H NMR (400 MHz, CD3CN) δ 8.67 (s, 1H), 7.87 (s, 2H), 7.23 (s, 1H), 6.89 (d, J=3.0 Hz, 1H), 6.82 (dd, J=8.6, 3.0 Hz, 1H), 3.84 (s, 2H), 3.73 (t, J=5.5 Hz, 2H), 3.24-3.16 (m, 4H), 3.10 (s, 3H), 3.02 (dt, J=4.9, 3.4 Hz, 3H), 2.65 (d, J=5.0 Hz, 1H), 2.58 (q, J=7.5 Hz, 2H), 1.14 (td, J=7.5, 1.9 Hz, 3H).

### Example 175: 7-(2-((7-ethyl-1,2,3,4-tetrahydroisoquinolin-6-yl)amino)-5-(trifluoromethyl)pyrimidin-4-yl)-4-(2,2,2-trifluoroethyl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(7-ethyl-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-(2,2,2-trifluoroethyl)-3,4-dihydrothieno[2,3-f][1,4]thiazepin-5(2H)-one 1,1-dioxide in step 3. The title compound was isolated. MS (ESI) m/z: 620.1 [M+H]⁺. ¹H NMR (400 MHz, methanol-d₄) δ 8.70 (d, J=1.6 Hz, 1H), 8.00 (s, 1H), 7.28-7.18 (m, 1H), 7.02 (s, 1H), 4.36 (d, J=9.2 Hz, 2H), 4.08 (t, J=5.6 Hz, 2H), 4.00 (s, 2H), 3.85-3.81 (m, 2H), 3.14-3.10 (m, 2H), 2.87 (t, J=5.6 Hz, 2H), 2.65-2.60 (m, 2H), 1.18-1.15 (m, 3H).

### Example 176: 4-methyl-7-(2-((5-methyl-1-(piperidin-4-yl)-1H-pyrazol-4-yl)amino)-5-(trifluoromethyl)pyrimidin-4-yl)-3,4-dihydrothieno[2,3-f][1,4]thiazepin-5(2H)-one 1,1-dioxide

The title compound was prepared following the synthetic procedure described in Example 166. The title compound was isolated. MS (ESI) m/z: 556.0 [M+H]⁺. ¹H NMR (400 MHz, methanol-d₄) δ 8.58 (s, 1H), 7.96 (s, 1H), 7.50 (s, 1H), 4.49-4.33 (m, 1H), 3.97-3.89 (m, 2H), 3.84-3.76 (m, 2H), 3.17 (s, 3H), 2.92 (dt, J=2.9, 12.8 Hz, 2H), 2.21 (s, 3H), 2.19-2.13 (m, 2H), 2.06 (d, J=10.0 Hz, 2H), 1.33-1.23 (m, 2H).

### Example 177: 7-(2-((6-cyclopropylisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 170, where 1-(5-amino-4-bromoisoindolin-2-yl)-2,2,2-trifluoroethan-1-one was replaced with 1-(5-amino-6-bromoisoindolin-2-yl)-2,2,2-trifluoroethan-1-one in step 2. The title compound was isolated. MS (ESI) m/z: 550.1 [M+H]⁺. ¹H NMR (400 MHz, methanol-d₄) δ 8.73 (s, 1H), 8.00 (s, 1H), 7.58 (s, 1H), 7.06 (s, 1H), 4.27 (s, 2H), 4.23 (s, 2H), 3.95-3.93 (m, 2H), 3.86-3.84 (m, 2H), 3.19 (s, 3H), 2.00-1.96 (m, 1H), 0.97-0.92 (m, 2H), 0.67-0.63 (m, 2H).

### Example 178: 7-(2-((5-methyl-1-(piperidin-4-yl)-1H-pyrazol-4-yl)amino)-5-(trifluoromethyl)pyrimidin-4-yl)-3,4-dihydrothieno[2,3-f][1,4]thiazepin-5(2H)-one 1,1-dioxide

The title compound was prepared using the same synthetic procedure described in the preparation of Example 165. The title compound was isolated. MS (ESI) m/z: 542.1 [M+H]⁺. ¹H NMR (400 MHz, methanol-d₄) δ 8.58 (s, 1H), 8.02 (s, 1H), 7.49 (s, 1H), 4.38 (m, 1H), 3.76 (d, J=2.8 Hz, 4H), 3.24 (s, 2H), 2.88-2.80 (m, 2H), 2.21 (s, 3H), 2.13 (m, 2H), 2.04-2.00 (m, 2H).

### Example 179: 7-(2-((2-ethyl-6-methylisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide

### Step 1: 1-(5-amino-6-bromoisoindolin-2-yl)-2,2,2-trifluoroethan-1-one

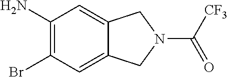 [SMILES: Nc1cc2c(cc1Br)CN(C(=O)C(F)(F)F)C2]

To a 0° C. solution 1-(5-aminoisoindolin-2-yl)-2,2,2-trifluoroethan-1-one (27 mmol) in acetonitrile (100 mL) was added N-bromosuccinimide (27 mmol) and the reaction mixture was stirred for 2 hours. The mixture was concentrated under vacuum and the residue was purified by silica gel chromatography (50% ethyl acetate in hexanes) to afford the title compound in 50% yield. MS (ESI) m/z: 308.9 [M+H]⁺. ¹H NMR (400 MHz, DMSO-d₆) ¹H NMR (400 MHz, DMSO-d₆) δ 7.38 (d, J=8.8 Hz, 1H), 7.19-6.96 (m, 1H), 6.85-6.66 (m, 1H), 5.41 (d, J=13.0 Hz, 2H), 5.02-4.81 (m, 2H), 4.80-4.56 (m, 2H).

### Step 2: 1-(5-amino-6-methylisoindolin-2-yl)-2,2,2-trifluoroethan-1-one

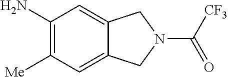 [SMILES: Cc1cc2c(cc1N)CN(C(=O)C(F)(F)F)C2]

The title compound was prepared analogously to Example 9, step 1, where 1-(7-chloro-6-nitro-1,2,3,4-tetrahydroisoquinolin-2-yl)-2,2,2-trifluoroethan-1-one and ethylboronic acid were replaced with 1-(5-amino-6-bromoisoindolin-2-yl)-2,2,2-trifluoroethan-1-one and 2,4,6-trimethyl-1,3,5,2,4,6-trioxatriborinane. The title compound was isolated in 28% yield. MS (ESI) m/z: 245.0 [M+H]⁺. ¹H NMR (400 MHz, DMSO-d₆) δ 6.92 (d, J=10.8 Hz, 1H), 6.57 (d, J=10.5 Hz, 1H), 4.93 (s, 2H), 4.85 (d, J=14.1 Hz, 2H), 4.66 (d, J=12.3 Hz, 2H), 2.06 (s, 3H).

### Steps 3-5: 7-(2-((2-ethyl-6-methylisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 170, steps 3-5, where 1-(5-amino-4-cyclopropylisoindolin-2-yl)-2,2,2-trifluoroethan-1-one was replaced with 1-(5-amino-6-methylisoindolin-2-yl)-2,2,2-trifluoroethan-1-one. The title compound was isolated. MS (ESI) m/z: 524.1 [M+H]⁺. ¹H NMR (400 MHz, methanol-d₄) δ 8.77 (s, 1H), 8.02 (s, 1H), 7.65 (s, 1H), 7.38 (s, 1H), 4.63 (d, J=7.0 Hz, 4H), 3.99-3.95 (m, 2H), 3.90-3.86 (m, 2H), 3.21 (s, 3H), 2.36 (s, 3H).

### Example 180: 7-(2-((6-chloroisoindolin-5-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(5-chloro-6-((5-(trifluoromethyl)-4-(trimethylstannyl)pyrimidin-2-yl)amino)isoindolin-2-yl)-2,2,2-trifluoroethan-1-one and 7-bromo-3,4-dihydrothieno[2,3-f][1,4]thiazepin-5(2H)-one 1,1-dioxide in step 3. The title compound was isolated. MS (ESI) m/z: 530.0 [M+H]⁺. ¹H NMR (400 MHz, methanol-d₄) δ=8.79 (s, 1H), 8.06 (s, 1H), 7.81 (s, 1H), 7.45 (s, 1H), 4.22 (d, J=11.5 Hz, 4H), 3.84-3.76 (m, 4H).

### Example 181: 7-(2-((6-chloro-2-methylisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 7-(2-((6-chloroisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 32% yield. m/z (ESI, +ve)=558.0 [M+H]⁺. ¹H NMR (400 MHz, methanol-d₄) δ=8.79 (s, 1H), 8.01 (s, 1H), 7.77 (s, 1H), 7.41 (s, 1H), 3.98-3.92 (m, 6H), 3.87-3.83 (m, 2H), 3.19 (s, 3H), 2.61 (s, 3H).

### Example 182: 7-(2-((3-methyl-1-(1-methylpiperidin-4-yl)-1H-pyrazol-4-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 7-(2-((3-methyl-1-(piperidin-4-yl)-1H-pyrazol-4-yl)amino)-5-(trifluoromethyl)pyrimidin-4-yl)-3,4-dihydrothieno[2,3-f][1,4]thiazepin-5(2H)-one 1,1-dioxide. The title compound was isolated in 10% yield. m/z (ESI, +ve)=555.9 [M+H]⁺. ¹H NMR (400 MHz, methanol-d₄) δ=8.59 (s, 1H), 8.08 (s, 1H), 7.88 (s, 1H), 4.29-4.17 (m, 1H), 3.81-3.75 (m, 5H), 3.17 (br d, J=12.4 Hz, 2H), 2.47 (s, 4H), 2.28-2.22 (m, 2H), 2.20-2.16 (m, 5H).

### Example 183: 4-methyl-7-(2-((3-methyl-1-(1-methylpiperidin-4-yl)-1H-pyrazol-4-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 4-methyl-7-(2-((3-methyl-1-(piperidin-4-yl)-1H-pyrazol-4-yl)amino)-5-(trifluoromethyl)pyrimidin-4-yl)-3,4-dihydrothieno[2,3-f][1,4]thiazepin-5(2H)-one 1,1-dioxide. The title compound was isolated in 47% yield. m/z (ESI, +ve)=570.1 [M+H]⁺. ¹H NMR (400 MHz, methanol-d₄) δ 8.58 (s, 1H), 8.02 (s, 1H), 7.86 (s, 1H), 4.21-4.09 (m, 1H), 3.96-3.88 (m, 2H), 3.86-3.76 (m, 2H), 3.18 (s, 3H), 3.07 (d, J=11.9 Hz, 2H), 2.38 (s, 3H), 2.36-2.25 (m, 2H), 2.24-2.18 (m, 2H), 2.17 (s, 3H), 2.16-2.05 (m, 2H).

### Example 184: 4-methyl-7-(2-((7-methyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 138, where diethylzinc was replaced with methylboronic acid in step 1 and 7-bromo-3,4-dihydrothieno[2,3-f][1,4]thiazepin-5(2H)-one 1,1-dioxide and 1-(7-ethyl-6-((5-(trifluoromethyl)-4-(trimethylstannyl)pyrimidin-2-yl)amino)-3,4-dihydroisoquinolin-2(1H)-yl)-2,2,2-trifluoroethan-1-one with 1-(6-{[4-chloro-5-(trifluoromethyl)pyrimidin-2-yl]amino}-7-methyl-1,2,3,4-tetrahydroisoquinolin-2-yl)-2,2,2-trifluoroethan-1-one and 4-methyl-7-(trimethylstannyl)-2H,3H,4H,5H-1λ⁶-thieno[2,3-f][1,4]thiazepine-1,1,5-trione, respectively, in step 5. The title compound was isolated. m/z (ESI, +ve)=538.2 [M+H]⁺. ¹H NMR (400 MHz, DMSO-d₆) δ 2.15 (s, 3H), 2.66 (m, 2H), 2.93 (t, J=5.8 Hz, 2H), 3.07 (s, 3H), 3.81 (s, 2H), 3.89 (m, 2H), 3.96 (m, 2H), 6.91 (s, 1H), 7.09 (s, 1H), 7.81 (s, 1H), 8.81 (s, 1H), 9.82 (s, 1H).

### Example 185: 7-(2-((2-ethyl-7-methyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-methyl-7-(2-((7-methyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and acetaldehyde. The title compound was isolated in 64% yield. m/z (ESI, +ve)=566.2 [M+H]⁺. ¹H NMR (300 MHz, CDCl₃) δ 1.26 (t, J=7.2 Hz, 3H), 2.27 (s, 3H), 2.70 (d, J=7.2 Hz, 2H), 2.88 (m, 2H), 3.00 (m, 2H), 3.23 (s, 3H), 3.72 (m, 4H), 3.90 (t, J=5.7 Hz, 2H), 6.94 (s, 1H), 7.18 (s, 1H), 7.58 (s, 1H), 8.06 (s, 1H), 8.69 (s, 1H).

### Example 186: 7-(2-((7-cyclopropyl-2-ethyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-cyclopropyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide and acetaldehyde. The title compound was isolated in 19% yield. ¹H NMR (400 MHz, DMSO-d₆) δ 0.56 (m, 2H), 0.81 (m, 2H), 1.09 (t, J=7.1 Hz, 3H), 1.93 (s, 1H), 2.47 (d, J=7.4 Hz, 2H), 2.62 (t, J=5.9 Hz, 2H), 2.77 (m, 2H), 3.07 (s, 3H), 3.49 (s, 2H), 3.88 (m, 2H), 3.95 (m, 2H), 6.69 (s, 1H), 7.15 (s, 1H), 7.81 (s, 1H), 8.81 (s, 1H), 9.86 (s, 1H).

### Example 187: 7-(2-((1-(2-(dimethylamino)ethyl)-3-methyl-1H-pyrazol-4-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 123, where 1-Boc-3-hydroxyazetidine was replaced with tert-butyl (2-hydroxyethyl)(methyl)carbamate in step 1 and tert-butyl 3-(4-((4-chloro-5-(trifluoromethyl)pyrimidin-2-yl)amino)-3-methyl-1H-pyrazol-1-yl)azetidine-1-carboxylate in step 1 and trimethyl(4-(methylsulfonyl)thiophen-2-yl)stannane with 4-methyl-7-(trimethylstannyl)-3,4-dihydrothieno[2,3-f][1,4]thiazepin-5(2H)-one 1,1-dioxide in step 4. The title compound was isolated. m/z (ESI, +ve)=543.9 [M+H]⁺. ¹H NMR (400 MHz, DMSO-d₆) δ 9.89 (m, 1H), 8.83 (m, 1H), 7.89 (m, 1H), 7.84 (s, 1H), 4.11 (t, J=6.5 Hz, 2H), 4.03-3.85 (m, 4H), 3.09 (d, J=8.0 Hz, 3H), 2.64 (t, J=6.6 Hz, 2H), 2.18 (s, 6H), 2.13 (d, J=9.9 Hz, 3H).

### Example 188: 7-(2-((2-ethyl-7-(methylthio)-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-methyl-7-(2-((7-(methylthio)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and acetaldehyde. The title compound was isolated in 23% yield. m/z (ESI, +ve)=598.2 [M+H]⁺. ¹H NMR (300 MHz, DMSO-d₆) δ 9.84 (s, 1H), 8.82 (s, 1H), 7.81 (s, 1H), 7.15 (s, 1H), 7.11 (m, 1H), 3.92 (m, 4H), 3.58 (s, 2H), 3.07 (s, 3H), 2.78 (m, 2H), 2.66 (m, 2H), 2.37 (s, 3H), 1.11 (t, J=7.1 Hz, 3H).

### Example 189: 7-(2-((2-chloro-5-(piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 149, steps 3-6, where tert-butyl 4-(3-amino-4-chlorophenyl)piperidine-1-carboxylate was replaced with tert-butyl 4-(3-amino-4-chlorophenyl)piperazine-1-carboxylate in step 3. The title compound was isolated.

MS (ESI) m/z: 587.0 [M+H]⁺. ¹H NMR (400 MHz, methanol-d₄) δ 8.83 (s, 1H), 8.02 (s, 1H), 7.76 (s, 1H), 7.37 (d, J=8.8 Hz, 1H), 6.87 (dd, J=2.8, 8.8 Hz, 1H), 4.83 (br d, J=3.6 Hz, 2H), 4.59 (s, 2H), 3.99-3.93 (m, 2H), 3.90-3.84 (m, 2H), 3.44-3.38 (m, 4H), 3.20 (s, 3H).

### Example 190: 7-(2-((2-ethyl-6-fluoroisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((6-fluoroisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide and acetaldehyde. The title compound was isolated in 11% yield. m/z (ESI, +ve)=556.2 [M+H]⁺. ¹H NMR (400 MHz, methanol-d₄) δ 8.80 (s, 1H), 8.01 (s, 1H), 7.85 (d, J=6.8 Hz, 1H), 7.19 (d, J=10.4 Hz, 1H), 4.58 (s, 2H), 4.19 (d, J=5.6 Hz, 2H), 3.94 (d, J=5.6 Hz, 2H), 3.89-3.84 (m, 2H), 3.19 (s, 3H), 3.08-2.98 (m, 2H), 1.32-1.27 (m, 3H).

### Example 191: 7-(2-((4-cyclopropyl-2-ethylisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((4-cyclopropylisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide and acetaldehyde. The title compound was isolated in 52% yield. m/z (ESI, +ve)=578.1 [M+H]⁺. ¹H NMR (400 MHz, methanol-d₄) δ 8.73 (s, 1H), 7.99 (s, 1H), 7.49 (d, J=8.0 Hz, 1H), 7.18 (d, J=8.0 Hz, 1H), 4.10 (s, 2H), 3.96 (s, 2H), 3.94-3.92 (m, 2H), 3.85-3.83 (m, 2H), 3.18 (s, 3H), 2.89-2.84 (m, 2H), 1.82-1.78 (m, 1H), 1.25 (t, J=7.2 Hz, 3H), 0.91-0.87 (m, 2H), 0.55-0.51 (m, 2H).

### Example 192: 7-(2-((2-ethyl-6-methylisoindolin-5-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-methyl-7-(2-((6-methylisoindolin-5-yl)amino)-5-(trifluoromethyl)pyrimidin-4-yl)-3,4-dihydrothieno[2,3-f][1,4]thiazepin-5(2H)-one 1,1-dioxide and acetaldehyde. The title compound was isolated in 52% yield. m/z (ESI, +ve)=552.2 [M+H]⁺. ¹H NMR (400 MHz, methanol-d₄) δ 8.72 (d, J=0.9 Hz, 1H), 8.00 (s, 1H), 7.39 (s, 1H), 7.21 (s, 1H), 4.04-3.92 (m, 6H), 3.88-3.81 (m, 2H), 3.20 (s, 3H), 2.86 (q, J=7.3 Hz, 2H), 2.30 (s, 3H), 1.25 (t, J=7.3 Hz, 3H).

### Example 193: 7-(2-((2-chloro-4-(piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 189, where tert-butyl 4-(3-amino-4-chlorophenyl)piperazine-1-carboxylate was replaced with tert-butyl 4-(4-amino-3-chlorophenyl)piperazine-1-carboxylate. The title compound was isolated. m/z (ESI, +ve)=586.9 [M+H]⁺. ¹H NMR ¹H NMR (400 MHz, acetonitrile-d₃) δ 8.75 (s, 1H), 7.92 (s, 1H), 7.58 (d, J=8.9 Hz, 1H), 7.06 (d, J=2.8 Hz, 1H), 6.96 (dd, J=9.0, 2.8 Hz, 1H), 3.90-3.85 (m, 2H), 3.77 (dd, J=6.5, 4.8 Hz, 2H), 3.13 (d, J=5.7 Hz, 8H), 2.96-2.91 (m, 4H).

### Example 194: 7-(2-((2-chloro-4-(4-methylpiperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 7-(2-((2-chloro-4-(piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated. m/z (ESI, +ve)=602.0 [M+H]⁺. ¹H NMR (400 MHz, acetonitrile-d₃) δ 8.76 (s, 1H), 8.00 (s, 1H), 7.92 (s, 1H), 7.59 (d, J=8.9 Hz, 1H), 7.07 (d, J=2.8 Hz, 1H), 6.97 (dd, J=9.0, 2.9 Hz, 1H), 3.91-3.85 (m, 2H), 3.77 (t, J=5.7 Hz, 2H), 3.25-3.20 (m, 4H), 3.14 (s, 3H), 2.56-2.50 (m, 4H), 2.30 (s, 3H).

### Example 195: 7-(2-((2-chloro-4-(4-ethylpiperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((2-chloro-4-(piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide and acetaldehyde. The title compound was isolated. m/z (ESI, +ve)=615.1 [M+H]⁺. ¹H NMR (400 MHz, acetonitrile-d₃) δ 8.75 (s, 1H), 8.00 (s, 1H), 7.92 (s, 1H), 7.59 (d, J=8.9 Hz, 1H), 7.07 (d, J=2.8 Hz, 1H), 6.98 (dd, J=9.0, 2.8 Hz, 1H), 3.88 (t, J=5.7 Hz, 2H), 3.77 (dd, J=6.5, 4.8 Hz, 2H), 3.26-3.19 (m, 4H), 3.13 (s, 3H), 2.58 (t, J=5.1 Hz, 4H), 2.49-2.42 (m, 2H), 1.10 (t, J=7.2 Hz, 3H).

### Example 196: 7-(2-((2-ethyl-4-(4-methylpiperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 7-(2-((2-ethyl-4-(piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 36% yield. m/z (ESI, +ve)=595.4 [M+H]⁺. ¹H NMR (400 MHz, acetonitrile-d₃) δ 8.66 (s, 1H), 7.87 (s, 2H), 7.22 (d, J=8.6 Hz, 1H), 6.88 (d, J=2.9 Hz, 1H), 6.81 (dd, J=8.7, 2.9 Hz, 1H), 3.84 (s, 2H), 3.73 (t, J=5.7 Hz, 2H), 3.22-3.14 (m, 4H), 3.10 (s, 3H), 2.57 (q, J=7.5 Hz, 2H), 2.53-2.46 (m, 4H), 2.25 (s, 3H), 1.13 (t, J=7.5 Hz, 3H).

### Example 197: 7-(2-((2-ethyl-4-(4-ethylpiperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((2-ethyl-4-(piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide and acetaldehyde. The title compound was isolated in 64% yield. m/z (ESI, +ve)=609.2 [M+H]⁺. ¹H NMR (300 MHz, DMSO-d₆) δ 9.69 (s, 1H), 8.78 (s, 1H), 7.79 (s, 1H), 7.13 (s, 1H), 6.91-6.71 (m, 2H), 3.94 (s, 3H), 3.21-2.97 (m, 9H), 2.37 (q, J=7.2 Hz, 2H), 1.06 (dt, J=13.2, 7.3 Hz, 7H). Two protons of methylene groups are masked by DMSO signal at 2.5 ppm.

### Example 198: 7-(2-((7-cyclopropyl-1,2,3,4-tetrahydroisoquinolin-6-yl)amino)-5-(trifluoromethyl)pyrimidin-4-yl)-4-ethyl-3,4-dihydrothieno[2,3-f][1,4]thiazepin-5(2H)-one 1,1-dioxide

### Step 1: 4-ethyl-7-(trimethylstannyl)-3,4-dihydrothieno[2,3-f][1,4]thiazepin-5(2H)-one 1,1-dioxide

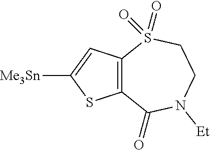

The title compound was prepared analogously to Example 160, step 1 where 7-bromo-4-methyl-3,4-dihydrothieno[2,3-f][1,4]thiazepin-5(2H)-one 1,1-dioxide was replaced with appropriate starting material. The title compound was isolated in 99% yield. MS (ESI) m/z: 325.9 [M+H]⁺.

### Step 2: 7-(2-((7-cyclopropyl-1,2,3,4-tetrahydroisoquinolin-6-yl)amino)-5-(trifluoromethyl)pyrimidin-4-yl)-4-ethyl-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 4-ethyl-7-(trimethylstannyl)-3,4-dihydrothieno[2,3-f][1,4]thiazepin-5(2H)-one 1,1-dioxide and 1-(6-((4-chloro-5-(trifluoromethyl)pyrimidin-2-yl)amino)-7-cyclopropyl-3,4-dihydroisoquinolin-2(1H)-yl)-2,2,2-trifluoroethan-1-one in step 3. The title compound was isolated. MS (ESI) m/z: 578.2 [M+H]⁺. ¹H NMR (400 MHz, DMSO-d₆) δ 9.86 (s, 1H), 8.81 (s, 1H), 7.82 (s, 1H), 7.11 (s, 1H), 6.65 (s, 1H), 3.91 (d, J=10.2 Hz, 4H), 3.80 (s, 2H), 3.53 (q, J=7.1 Hz, 2H), 2.92 (t, J=5.8 Hz, 2H), 2.65 (q, J=6.5, 5.7 Hz, 2H), 1.92 (d, J=5.2 Hz, 1H), 1.17 (t, J=7.1 Hz, 3H), 0.87-0.76 (m, 2H), 0.56 (dt, J=5.3, 2.9 Hz, 2H).

### Example 199: 7-(2-((7-cyclopropyl-2-ethyl-1,2,3,4-tetrahydroisoquinolin-6-yl)amino)-5-(trifluoromethyl)pyrimidin-4-yl)-4-ethyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-cyclopropyl-1,2,3,4-tetrahydroisoquinolin-6-yl)amino)-5-(trifluoromethyl)pyrimidin-4-yl)-4-ethyl-3,4-dihydrothieno[2,3-f][1,4]thiazepin-5(2H)-one 1,1-dioxide and acetaldehyde. The title compound was isolated in 69% yield. m/z (ESI, +ve)=606.2 [M+H]⁺. ¹H NMR (400 MHz, DMSO-d₆) δ 9.87 (s, 1H), 8.82 (s, 1H), 7.82 (s, 1H), 7.15 (s, 1H), 6.70 (s, 1H), 3.91 (d, J=10.7 Hz, 4H), 3.56-3.47 (m, 4H), 2.77 (s, 2H), 2.63 (t, J=5.8 Hz, 2H), 1.93 (s, 1H), 1.13 (dt, J=27.4, 7.1 Hz, 6H), 0.85-0.79 (m, 2H), 0.60-0.53 (m, 2H).

### Example 200: 7-(2-((6-chloro-1-methyl-1H-benzo[d][1,2,3]triazol-5-yl)amino)-5-(trifluoromethyl)pyrimidin-4-yl)-4-methyl-3,4-dihydrothieno[2,3-f][1,4]thiazepin-5(2H)-one 1,1-dioxide

### Step 1: 5-chloro-N1-methyl-4-nitrobenzene-1,2-diamine

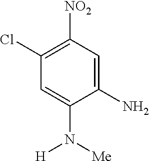 [SMILES: CNc1cc(Cl)c([N+](=O)[O-])cc1N]

To a solution of 4-chloro-2-fluoro-5-nitroaniline (33 mmol) and methylamine hydrochloride (130 mmol) in acetonitrile (100 mL) was added triethylamine (198 mmol). The mixture was stirred at 100° C. for 8 hours. The reaction mixture was diluted with water, extracted with ethyl acetate three times and the combined organic layers were dried over anhydrous sodium sulfate, filtered, and concentrated under reduced pressure to afford a residue that was purified by silica gel chromatography (30% ethyl acetate in hexanes). The title compound was isolated in 53% yield. ¹H NMR (400 MHz, CDCl₃) δ=7.55 (s, 1H), 6.58 (s, 1H), 2.96 (s, 3H).

### Step 2: 6-chloro-1-methyl-5-nitro-1H-benzo[d][1,2,3]triazole

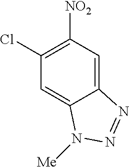 [SMILES: Cn1nnc2cc([N+](=O)[O-])c(Cl)cc21]

To a 0° C. solution of 5-chloro-N1-methyl-4-nitrobenzene-1,2-diamine (17 mmol) in acetic acid (22 mL) and water (11 mL) was added sodium nitrite (26 mmol). After 1 hour the reaction mixture was diluted with water, extracted with ethyl acetate three times and the combined organic layers were dried over anhydrous sodium sulfate, filtered, and concentrated under reduced pressure to afford the title compound in 79% yield. ¹H NMR (400 MHz, CDCl₃) δ=8.63 (s, 1H), 7.74 (s, 1H), 4.36 (s, 3H).

### Step 3: 6-chloro-1-methyl-1H-benzo[d][1,2,3]triazol-5-amine

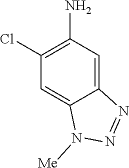 [SMILES: Cn1nnc2cc(N)c(Cl)cc21]

To a solution of 6-chloro-1-methyl-5-nitro-1H-benzo[d][1,2,3]triazole (14 mmol) in ethanol (40 mL) and water (15 mL) was added ammonium hydrochloride (27 mmol) and iron (68 mmol). The mixture was stirred at 80° C. for 2 hours, cooled down to room temperature and filtered. The filtrate was concentrated under reduced pressure to afford the title compound in 72% yield. MS (ESI) m/z: 183.2 [M+H]⁺.

### Step 4: 6-chloro-N-(4-chloro-5-(trifluoromethyl)pyrimidin-2-yl)-1-methyl-1H-benzo[d][1,2,3]triazol-5-amine

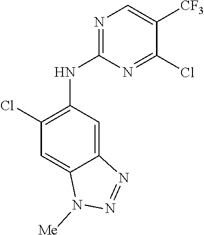 [SMILES: Cn1nnc2cc(Nc3ncc(C(F)(F)F)c(Cl)n3)c(Cl)cc21]

The title compound was prepared analogously to Example 1, step 5 where 1-(6-amino-7-chloro-1,2,3,4-tetrahydroisoquinolin-2-yl)-2,2,2-trifluoroethan-1-one was replaced with 6-chloro-1-methyl-1H-benzo[d][1,2,3]triazol-5-amine. The title compound was isolated in 20% yield.

MS (ESI) m/z: 363.0 [M+H]⁺.

### Step 5: 6-chloro-1-methyl-N-(5-(trifluoromethyl)-4-(trimethylstannyl)pyrimidin-2-yl)-1H-benzo[d][1,2,3]triazol-5-amine

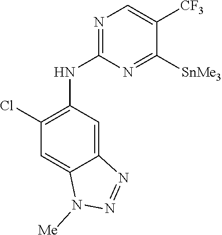

The title compound was prepared analogously to Example 4, step 1 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one (3.26 mmol), 1,4-bis(diphenylphosphino)butane was replaced with 6-chloro-N-(4-chloro-5-(trifluoromethyl)pyrimidin-2-yl)-1-methyl-1H-benzo[d][1,2,3]triazol-5-amine. The title compound was isolated.

### Step 6: 7-(2-((6-chloro-1-methyl-1H-benzo[d][1,2,3]triazol-5-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 155, step 3, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 6-chloro-1-methyl-N-(5-(trifluoromethyl)-4-(trimethylstannyl)pyrimidin-2-yl)-1H-benzo[d][1,2,3]triazol-5-amine and 7-bromo-4-methyl-3,4-dihydrothieno[2,3-f][1,4]thiazepin-5(2H)-one 1,1-dioxide. The title compound was isolated. MS (ESI) m/z: 558.2 [M+H]⁺. ¹H NMR (400 MHz, DMSO-d₆) δ=10.28 (brs, 1H), 8.88 (s, 1H), 8.26 (d, J=13.6 Hz, 2H), 7.82 (s, 1H), 4.33 (s, 3H), 3.98-3.82 (m, 4H), 3.05 (s, 3H).

### Example 201: 7-(2-((6-chloro-1,2,3,4-tetrahydroisoquinolin-7-yl)amino)-5-(trifluoromethyl)pyrimidin-4-yl)-4-cyclopropyl-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one was replaced with 1-(6-chloro-7-((5-(trifluoromethyl)-4-(trimethylstannyl)pyrimidin-2-yl)amino)-3,4-dihydroisoquinolin-2(1H)-yl)-2,2,2-trifluoroethan-1-one in step 3. The title compound was isolated. MS (ESI) m/z: 584.1 [M+H]⁺. ¹H NMR (400 MHz, DMSO-d₆) δ 10.05 (br s, 1H), 8.85 (s, 1H), 7.80 (br s, 1H), 7.34-7.17 (m, 2H), 3.92-3.84 (m, 4H), 3.84-3.77 (m, 2H), 2.96-2.90 (m, 2H), 2.89-2.83 (m, 1H), 2.73-2.68 (m, 2H), 2.55 (br s, 1H), 0.83-0.75 (m, 4H).

### Example 202: 7-(2-((6-chloro-1,2,3,4-tetrahydroisoquinolin-7-yl)amino)-5-(trifluoromethyl)pyrimidin-4-yl)-4-cyclobutyl-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(6-chloro-7-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclobutyl-3,4-dihydrothieno[2,3-f][1,4]thiazepin-5(2H)-one 1,1-dioxide in step 3. The title compound was isolated. MS (ESI) m/z: 598.1 [M+H]⁺. ¹H NMR (400 MHz, DMSO-d₆) δ 10.05 (s, 1H), 8.86 (s, 1H), 7.82 (s, 1H), 7.33-7.20 (m, 2H), 4.73-4.61 (m, 1H), 3.95-3.89 (m, 2H), 3.89-3.84 (m, 2H), 3.84-3.77 (m, 2H), 2.97-2.87 (m, 2H), 2.72-2.67 (m, 2H), 2.47-2.45 (m, 1H), 2.26-2.18 (m, 2H), 2.17-2.10 (m, 2H), 1.73-1.61 (m, 2H).

### Example 203: 7-(2-((7-chloro-2-ethyl-1,2,3,4-tetrahydroisoquinolin-6-yl)amino)-5-(trifluoromethyl)pyrimidin-4-yl)-4-cyclobutyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-chloro-1,2,3,4-tetrahydroisoquinolin-6-yl)amino)-5-(trifluoromethyl)pyrimidin-4-yl)-4-cyclobutyl-3,4-dihydrothieno[2,3-f][1,4]thiazepin-5(2H)-one 1,1-dioxide and acetaldehyde. The title compound was isolated in 35% yield. m/z (ESI, +ve)=626.1 [M+H]⁺. ¹H NMR (400 MHz, DMSO-d₆) δ 10.07 (s, 1H), 8.86 (s, 1H), 7.82 (s, 1H), 7.37-7.27 (m, 2H), 4.70-4.63 (m, 1H), 3.93-3.89 (m, 2H), 3.89-3.82 (m, 3H), 3.65-3.54 (m, 1H), 3.30-3.28 (m, 4H), 2.88-2.77 (m, 2H), 2.24-2.18 (m, 1H), 2.16-2.12 (m, 2H), 1.70-1.64 (m, 2H), 1.14-1.09 (m, 3H).

### Example 204: 7-(2-((6-chloro-1,2,3,4-tetrahydroisoquinolin-7-yl)amino)-5-(trifluoromethyl)pyrimidin-4-yl)-4-ethyl-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 4-ethyl-7-(trimethylstannyl)-3,4-dihydrothieno[2,3-f][1,4]thiazepin-5(2H)-one 1,1-dioxide and 1-(6-chloro-7-((4-chloro-5-(trifluoromethyl)pyrimidin-2-yl)amino)-3,4-dihydroisoquinolin-2(1H)-yl)-2,2,2-trifluoroethan-1-one in step 3. The title compound was isolated. MS (ESI) m/z: 572.1 [M+H]⁺. ¹H NMR (400 MHz, DMSO-d₆) δ 10.05 (s, 1H), 8.86 (s, 1H), 7.83 (s, 1H), 7.26 (d, J=13.8 Hz, 2H), 3.92 (dd, J=8.0, 4.5 Hz, 4H), 3.83 (s, 2H), 3.53 (q, J=7.2 Hz, 2H), 2.94 (t, J=5.8 Hz, 2H), 2.70 (t, J=5.7 Hz, 2H), 1.17 (t, J=7.1 Hz, 3H).

### Example 205: 7-(2-((2-(cyclopropylmethyl)-7-methyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

To a solution of 4-methyl-7-(2-((7-methyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide (0.22 mmol) in methanol (1.2 mL) and THF (1.2 mL), acetic acid (0.026 mL) and sodium cyanoborohydride (0.33 mmol) were added. The reaction mixture was cooled down to −30° C. and cyclopropanecarboxaldehyde (0.265 mmol) was added. The reaction mixture was stirred at −30° C. for 5 minutes, then the cooling bath was removed and the reaction mixture was allowed to reach room temperature and stirred for one additional hour. The reaction mixture was diluted with water, extracted with dichloromethane three times and the combined organic layers were dried over anhydrous sodium sulfate, filtered, and concentrated under reduced pressure to afford the title compound in 32% yield. MS (ESI) m/z: 592.2 [M+H]⁺. ¹H NMR (300 MHz, acetonitrile-d₃) δ 8.71 (s, 1H), 7.93 (d, J=17.6 Hz, 2H), 7.29 (s, 1H), 6.98 (s, 1H), 3.84 (d, J=6.1 Hz, 2H), 3.74 (t, J=5.8 Hz, 2H), 3.64 (s, 2H), 3.10 (s, 3H), 2.85 (t, J=5.9 Hz, 2H), 2.76 (t, J=6.0 Hz, 2H), 2.39 (d, J=6.6 Hz, 2H), 2.21 (s, 3H), 0.95 (s, 1H), 0.54 (d, J=7.8 Hz, 2H), 0.17 (d, J=5.0 Hz, 2H).

### Example 206: 7-(2-((2-chloro-4-(4-(cyclopropylmethyl)piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 205, where 4-methyl-7-(2-((7-methyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide was replaced with 7-(2-((2-chloro-4-(piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 51% yield. MS (ESI) m/z: 641.1 [M+H]⁺. ¹H NMR (400 MHz, acetonitrile-d₃) δ 8.72 (s, 1H), 7.98 (s, 1H), 7.89 (s, 1H), 7.56 (d, J=8.9 Hz, 1H), 7.04 (d, J=2.8 Hz, 1H), 6.95 (dd, J=8.9, 2.8 Hz, 1H), 3.88-3.80 (m, 2H), 3.74 (dd, J=6.5, 4.8 Hz, 2H), 3.22-3.17 (m, 4H), 3.11 (s, 3H), 2.65-2.59 (m, 4H), 2.25 (d, J=6.6 Hz, 2H), 0.92-0.81 (m, 1H), 0.55-0.44 (m, 2H), 0.16-0.07 (m, 2H).

### Example 207: 4-cyclopropyl-7-(2-((7-ethyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

### Step 1: 7-bromo-4-cyclopropyl-3,4-dihydrothieno[2,3-f][1,4]thiazepin-5(2H)-one 1,1-dioxide

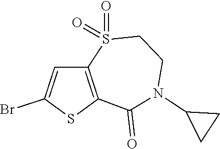 [SMILES: O=C1c2sc(Br)cc2S(=O)(=O)CCN1C1CC1]

To a solution of 7-bromo-1,1-dioxo-3,4-dihydro-2H-thieno[2,3-f][1,4]thiazepin-5-one (1.35 mmol) and cyclopropylboronic acid (4.05 mmol) in 1,2-dichloroethane (2 mL) and DMF (0.6 mL) was added copper diacetate (1.35 mmol), pyridine (8.10 mmol) and 4A molecular sieve (1.2 g). The mixture was stirred at 80° C. for 12 hours under air. The mixture was filtered and concentrated under vacuum to afford a residue that was purified by semi-preparative reverse phase-HPLC. The title compound was isolated in 66% yield. MS (ESI) m/z: 336.0[M+H]⁺. ¹H NMR (400 MHz, CDCl₃) δ 7.44 (s, 1H), 3.99-3.91 (m, 2H), 3.62 (dd, J=5.2, 6.4 Hz, 2H), 2.92-2.83 (m, 1H), 0.97-0.91 (m, 2H), 0.90-0.85 (m, 2H).

### Step 2: 4-cyclopropyl-7-(2-((7-ethyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one was replaced with 1-(7-ethyl-6-((5-(trifluoromethyl)-4-(trimethylstannyl)pyrimidin-2-yl)amino)-3,4-dihydroisoquinolin-2(1H)-yl)-2,2,2-trifluoroethan-1-one in step 3. The title compound was isolated. MS (ESI) m/z: 578.3 [M+H]⁺. ¹H NMR (400 MHz, methanol-d₄) δ 8.68 (d, J=1.2 Hz, 1H), 7.97 (s, 1H), 7.33-7.16 (m, 1H), 7.01 (s, 1H), 4.01-3.93 (m, 4H), 3.79-3.74 (m, 2H), 3.14 (t, J=5.6 Hz, 2H), 2.94-2.85 (m, 3H), 2.63 (q, J=7.6 Hz, 2H), 1.16 (t, J=7.6 Hz, 3H), 0.92-0.88 (m, 4H).

### Example 208: 4-cyclobutyl-7-(2-((7-ethyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(7-ethyl-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclobutyl-3,4-dihydrothieno[2,3-f][1,4]thiazepin-5(2H)-one 1,1-dioxide in step 3. The title compound was isolated. MS (ESI) m/z: 592.3 [M+H]⁺. ¹H NMR (400 MHz, methanol-d₄) δ=8.74-8.64 (m, 1H), 7.97 (s, 1H), 7.30-7.19 (m, 1H), 7.00 (s, 1H), 4.78-4.71 (m, 1H), 3.99 (s, 2H), 3.96-3.91 (m, 2H), 3.76 (m 2H), 3.14-3.10 (m, 2H), 2.88-2.82 (m, 2H), 2.62 (m, 2H), 2.33-2.25 (m, 4H), 1.82-1.73 (m, 2H), 1.15 (t, J=7.6 Hz, 3H).

### Example 209: 4-cyclopropyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 155, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one was replaced with 1-(7-cyclopropyl-6-((5-(trifluoromethyl)-4-(trimethylstannyl)pyrimidin-2-yl)amino)-3,4-dihydroisoquinolin-2(1H)-yl)-2,2,2-trifluoroethan-1-one in step 3. The title compound was isolated. MS (ESI) m/z: 590.1 [M+H]⁺. ¹H NMR (400 MHz, methanol-d₄) δ8.73 (s, 1H), 7.99 (s, 1H), 7.50-7.40 (m, 1H), 6.80 (s, 1H), 4.01-3.94 (m, 4H), 3.76 (m, 2H), 3.16-3.11 (m, 2H), 2.94-2.86 (m, 3H), 1.96-1.86 (m, 1H), 0.93-0.88 (m, 6H), 0.65-0.57 (m, 2H).

### Example 210: 7-(2-((7-chloro-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

### Step 1: 7-bromo-4-(oxetan-3-yl)-3,4-dihydrothieno[2,3-f][1,4]thiazepin-5(2H)-one 1,1-dioxide

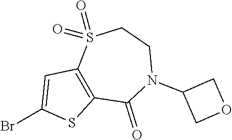 [SMILES: O=C1c2sc(Br)cc2S(=O)(=O)CCN1C1COC1]

A mixture of 7-bromo-1,1-dioxo-3,4-dihydro-2H-thieno[2,3-f][1,4]thiazepin-5-one (1.02 mmol), 3-bromooxetane (1.92 mmol), cesium carbonate (2.03 mmol) and DMF (5 mL) was stirred at 60° C. for 12 hours. The reaction mixture was poured into water and extracted with ethyl acetate three times. The combined organic layers were washed with saturated brine, dried over anhydrous sodium sulfate, filtered, and concentrated under reduced pressure to afford a crude product that was purified by preparative TLC (50% of ethyl acetate in hexanes). The title compound was isolated in 13% yield as a colorless oil. ¹H NMR (400 MHz, DMSO-d₆) δ 7.65 (s, 1H), 5.28-5.17 (m, 1H), 4.74-4.69 (m, 2H), 4.68-4.63 (m, 2H), 4.06-3.98 (m, 4H).

### Step 2: 7-(2-((7-chloro-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 155, steps 3-4, where 7-bromo-4-cyclopropyl-3,4-dihydrothieno[2,3-f][1,4]thiazepin-5(2H)-one 1,1-dioxide was replaced with 7-bromo-4-(oxetan-3-yl)-3,4-dihydrothieno[2,3-f][1,4]thiazepin-5(2H)-one 1,1-dioxide in step 3. The title compound was isolated. MS (ESI) m/z: 600.1 [M+H]⁺. ¹H NMR (400 MHz, DMSO-d₆) δ 10.08 (s, 1H), 8.86 (s, 1H), 7.84 (s, 1H), 7.29 (s, 1H), 7.24 (s, 1H), 5.27-5.18 (m, 1H), 4.76-4.67 (m, 4H), 4.05-3.98 (m, 4H), 3.88-3.84 (m, 2H), 2.98-2.92 (m, 2H), 2.69 (br t, J=5.6 Hz, 2H).

### Example 211: 7-(2-((6-chloro-2-ethyl-1,2,3,4-tetrahydroisoquinolin-7-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((6-chloro-1,2,3,4-tetrahydroisoquinolin-7-yl)amino)-5-(trifluoromethyl)pyrimidin-4-yl)-4-methyl-3,4-dihydrothieno[2,3-f][1,4]thiazepin-5(2H)-one 1,1-dioxide and acetaldehyde. The title compound was isolated in 69% yield. m/z (ESI, +ve)=586.1 [M+H]⁺. ¹H NMR (400 MHz, DMSO-d₆) δ 10.04 (br s, 1H), 8.85 (s, 1H), 7.82 (s, 1H), 7.31 (s, 2H), 3.98-3.92 (m, 2H), 3.91-3.84 (m, 2H), 3.57-3.50 (m, 2H), 3.09-3.03 (m, 3H), 2.85-2.79 (m, 2H), 2.68-2.65 (m, 2H), 2.53-2.52 (m, 2H), 1.08 (t, J=7.2 Hz, 3H).

### Example 212: 7-(2-((6-ethyl-1,2,3,4-tetrahydroisoquinolin-7-yl)amino)-5-(trifluoromethyl)pyrimidin-4-yl)-4-methyl-3,4-dihydrothieno[2,3-f][1,4]thiazepin-5(2H)-one 1,1-dioxide

### Step 1: 1-(6-bromo-3,4-dihydroisoquinolin-2(1H)-yl)-2,2,2-trifluoroethan-1-one

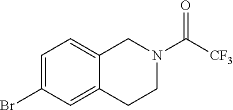 [SMILES: O=C(N1CCc2cc(Br)ccc2C1)C(F)(F)F]

A 0° C. solution of 6-bromo-1,2,3,4-tetrahydroisoquinoline (47 mmol) in dichloromethane (100 mL) was treated with pyridine (94 mmol), N,N-dimethylaminopyridine (2.4 mmol) and trifluoroacetic anhydride (57 mmol) and triethylamine (112 mmol). After 12 hours at room temperature, the reaction mixture was diluted with water, extracted with ethyl acetate three times and the combined organic layers were dried over anhydrous sodium sulfate, filtered, and concentrated under reduced pressure. The crude material was purified by silica gel chromatography (0-10% ethyl acetate in hexanes) to afford the title compound in 94% yield. MS (ESI) m/z: 307.9 [M+H]⁺. ¹H NMR (400 MHz, CDCl₃) δ 7.37-7.33 (m, 2H), 7.05-6.99 (m, 1H), 4.74-4.69 (m, 2H), 3.89-3.82 (m, 2H), 2.96-2.92 (m, 2H).

### Step 2: 1-(6-bromo-7-nitro-3,4-dihydroisoquinolin-2(1H)-yl)-2,2,2-trifluoroethan-1-one

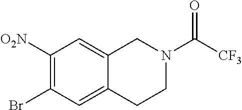 [SMILES: O=C(N1CCc2cc(Br)c([N+](=O)[O-])cc2C1)C(F)(F)F]

1-(6-bromo-3,4-dihydroisoquinolin-2(1H)-yl)-2,2,2-trifluoroethan-1-one (44 mmol) was dissolved in sulfuric acid (130 mL). The solution was cooled down to 0° C. and fuming nitric acid (2 mL) was added dropwise over 30 min while keeping the temperature at 0° C. After 60 minutes, the reaction was quenched with ice-water and the aqueous layer was extracted with ethyl acetate three times. The combined organic layers were dried over anhydrous sodium sulfate, filtered, and concentrated to afford the title compound in 95% yield. MS (ESI) m/z: 353.9 [M+H]⁺. ¹H NMR (400 MHz, CDCl₃) δ 7.69 (s, 1H), 7.57-7.55 (m, 1H), 4.78-4.75 (m, 2H), 3.91-3.85 (m, 2H), 3.02-2.97 (m, 2H).

### Step 3: 2,2,2-trifluoro-1-(7-nitro-6-vinyl-3,4-dihydroisoquinolin-2(1H)-yl)ethan-1-one

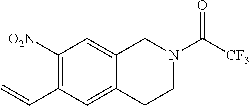 [SMILES: C=Cc1cc2c(cc1[N+](=O)[O-])CN(C(=O)C(F)(F)F)CC2]

A solution of 1-(6-bromo-7-nitro-3,4-dihydro-1H-isoquinolin-2-yl)-2,2,2-trifluoro-ethanone (17 mmol) and tributyl(vinyl)stannane (25 mmol) in dioxane (60 mL) was treated with tetrakis(triphenylphosphine) palladium(0) (1.7 mmol) and copper iodide (17 mmol). The mixture was stirred at 120° C. for 12 hours, cooled down to room temperature, filtered and concentrated under reduced pressure. The residue was purified by silica gel chromatography (0-6% ethyl acetate in hexanes). The title compound was isolated in 58% yield. MS (ESI) m/z: 301.0[M+H]⁺. ¹H NMR (400 MHz, DMSO-d₆) δ 8.04-7.98 (m, 1H), 7.65 (s, 1H), 7.04-6.96 (m, 1H), 5.88 (d, J=17.2 Hz, 1H), 5.49 (d, J=11.2 Hz, 1H), 4.88-4.83 (m, 2H), 3.84-3.82 (m, 2H), 3.05-3.00 (m, 2H).

### Step 4: 1-(7-amino-6-ethyl-3,4-dihydroisoquinolin-2(1H)-yl)-2,2,2-trifluoroethan-1-one

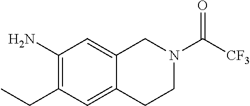 [SMILES: CCc1cc2c(cc1N)CN(C(=O)C(F)(F)F)CC2]

A solution of 2,2,2-trifluoro-1-(7-nitro-6-vinyl-3,4-dihydroisoquinolin-2(1H)-yl)ethan-1-one (9.33 mmol) in methanol (30 mL) was hydrogenated in the presence of 10% palladium on carbon (1.0 g) for two hours. The reaction was filtered through celite and concentrated. Purification by silica gel chromatography (0-12% ethyl acetate in hexanes) afforded the title compound in 85% yield. MS (ESI) m/z: 273.1[M+H]⁺. ¹H NMR (400 MHz, DMSO-d₆) δ 6.74-6.73 (m, 1H), 6.41 (s, 1H), 4.75 (s, 2H), 4.56 (s, 2H), 3.75-3.73 (m, 2H), 2.75-2.69 (m, 2H), 2.40 (q, J=14.8, 7.6 Hz, 2H), 1.09 (t, J=7.6 Hz, 3H).

### Step 5: 1-(7-((4-chloro-5-(trifluoromethyl)pyrimidin-2-yl)amino)-6-ethyl-3,4-dihydroisoquinolin-2(1H)-yl)-2,2,2-trifluoroethan-1-one

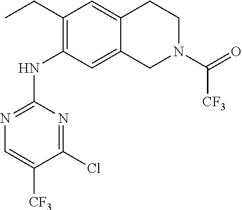 [SMILES: CCc1cc2c(cc1Nc1ncc(C(F)(F)F)c(Cl)n1)CN(C(=O)C(F)(F)F)CC2]

1-(7-amino-6-ethyl-3,4-dihydro-1H-isoquinolin-2-yl)-2,2,2-trifluoro-ethanone (7.0 mmol) and 2,4-dichloro-5-(trifluoromethyl)pyrimidine (35 mmol) were heated at 70° C. for 3 hours. The mixture was cooled down and purified by silica gel chromatography (0-10% ethyl acetate in hexanes). The title compound was isolated in 39% yield. MS (ESI) m/z: 453.1[M+H]⁺. ¹H NMR (400 MHz, DMSO-d₆) δ 10.13 (s, 1H), 8.65 (br s, 1H), 7.19-7.16 (m, 1H), 7.14-7.13 (m, 1H), 4.71-4.69 (m, 2H), 3.83-3.80 (m, 2H), 2.93-2.88 (m, 2H), 2.54-2.52 (m, 2H), 1.07 (t, J=7.6 Hz, 3H).

### Step 6: 1-(6-ethyl-7-((5-(trifluoromethyl)-4-(trimethylstannyl)pyrimidin-2-yl)amino)-3,4-dihydroisoquinolin-2(1H)-yl)-2,2,2-trifluoroethan-1-one

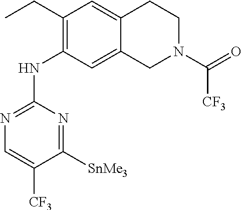

To a solution of 1-[7-[[4-chloro-5-(trifluoromethyl)pyrimidin-2-yl]amino]-6-ethyl-3,4-dihydro-1H-isoquinolin-2-yl]-2,2,2-trifluoro-ethanone (3.1 mmol) and trimethyl(trimethylstannyl)stannane (12.4 mmol) in dioxane (10 mL) was added palladium diacetate (0.62 mmol) and 1,4-bis(diphenylphosphino)butane (0.62 mmol). The mixture was stirred at 95° C. for 12 hours. The mixture was cooled down to room temperature and quenched with water, extracted with ethyl acetate and the combined organic layers were washed with brine and dried with anhydrous sodium sulfate, filtered and concentrated under reduced pressure. The residue was purified by neutral alumina chromatography (0-5% ethyl acetate in hexanes). The title compound was isolated in 62% yield. MS (ESI) m/z: 583.2[M+H]⁺. ¹H NMR (400 MHz, DMSO-d₆) δ 9.35-9.32 (m, 1H), 8.48 (s, 1H), 7.29 (s, 1H), 7.09 (d, J=3.2 Hz, 1H), 4.71-4.69 (m, 2H), 3.82-3.79 (m, 2H), 2.92-2.88 (m, 2H), 2.59-2.55 (m, 2H), 1.10-1.06 (m, 3H), 0.28 (d, J=4.0 Hz, 9H).

### Steps 7-8: 7-(2-((6-ethyl-1,2,3,4-tetrahydroisoquinolin-7-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(6-ethyl-7-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-methyl-3,4-dihydrothieno[2,3-f][1,4]thiazepin-5(2H)-one 1,1-dioxide in step 3. The title compound was isolated. MS (ESI) m/z: 552.3 [M+H]⁺. ¹H NMR (400 MHz, methanol-d₄) δ 8.68 (s, 1H), 7.98 (s, 1H), 7.18 (s, 1H), 7.07 (s, 1H), 4.00 (s, 2H), 3.95-3.92 (m, 2H), 3.85-3.83 (m, 2H), 3.18 (s, 3H), 3.13 (t, J=6.0 Hz, 2H), 2.86 (t, J=6.0 Hz, 2H), 2.63 (q, J=15.2, 7.2 Hz, 2H), 1.16 (t, J=7.6 Hz, 3H).

### Example 213: 7-(2-((7-chloro-2-ethyl-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-chloro-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide and acetaldehyde. The title compound was isolated in 69% yield. m/z (ESI, +ve)=612.2 [M+H]⁺. ¹H NMR (400 MHz, methanol-d₄) δ 8.83 (s, 1H), 8.01 (s, 1H), 7.94 (s, 1H), 7.43 (s, 1H), 4.39 (s, 2H), 4.02-3.97 (m, 2H), 3.79 (t, J=6.0 Hz, 2H), 3.56 (s, 2H), 3.35-3.32 (m, 2H), 3.28-3.23 (m, 2H), 2.94-2.87 (m, 1H), 1.45 (t, J=7.2 Hz, 3H), 0.93-0.90 (m, 4H).

### Example 214: 7-(2-((6-chloro-1,2,3,4-tetrahydroisoquinolin-7-yl)amino)-5-(trifluoromethyl)pyrimidin-4-yl)-4-(2,2,2-trifluoroethyl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 4-methyl-7-(trimethylstannyl)-2H,3H,4H,5H-1λ⁶-thieno[2,3-f][1,4]thiazepine-1,1,5-trione and 1-(6-chloro-7-((4-chloro-5-(trifluoromethyl)pyrimidin-2-yl)amino)-3,4-dihydroisoquinolin-2(1H)-yl)-2,2,2-trifluoroethan-1-one in step 3. The title compound was isolated. MS (ESI) m/z: 626.1 [M+H]⁺. ¹H NMR (400 MHz, DMSO-d₆) δ 10.08 (s, 1H), 8.87 (s, 1H), 7.85 (s, 1H), 7.28 (s, 1H), 7.25 (s, 1H), 4.45 (q, J=9.5 Hz, 2H), 4.08 (t, J=5.7 Hz, 2H), 3.93 (t, J=5.6 Hz, 2H), 3.83 (s, 2H), 2.93 (t, J=5.8 Hz, 2H), 2.71 (t, J=5.9 Hz, 2H).

### Example 215: 7-(2-((7-cyclopropyl-1,2,3,4-tetrahydroisoquinolin-6-yl)amino)-5-(trifluoromethyl)pyrimidin-4-yl)-4-(2,2,2-trifluoroethyl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 4-methyl-7-(trimethylstannyl)-2H,3H,4H,5H-1λ⁶-thieno[2,3-f][1,4]thiazepine-1,1,5-trione and 1-(6-((4-chloro-5-(trifluoromethyl)pyrimidin-2-yl)amino)-7-cyclopropyl-3,4-dihydroisoquinolin-2(1H)-yl)-2,2,2-trifluoroethan-1-one in step 3. The title compound was isolated. MS (ESI) m/z: 632.2 [M+H]⁺. ¹H NMR (400 MHz, DMSO-d₆) δ 9.89 (s, 1H), 8.83 (s, 1H), 7.84 (s, 1H), 7.09 (s, 1H), 6.65 (s, 1H), 4.45 (q, J=9.5 Hz, 2H), 4.07 (s, 2H), 3.94 (d, J=5.9 Hz, 2H), 3.80 (s, 2H), 2.92 (t, J=5.8 Hz, 2H), 2.65 (s, 2H), 0.86-0.77 (m, 2H), 0.59-0.50 (m, 2H).

### Example 216: 4-methyl-7-(2-((7-methyl-2-(oxetan-3-ylmethyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

To a solution of 4-methyl-7-(2-((7-methyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide (0.22 mmol) in methanol (1.2 mL) and THF (1.2 mL), acetic Acid (0.44 mmol) and sodium cyanoborohydride (0.33 mmol) were added. The reaction mixture was cooled down to −30° C. and oxetane-3-carbaldehyde (0.25 mmol) was added. The reaction mixture was stirred at −30° C. for 5 minutes and at room temperature for one hour. The reaction mixture was diluted with water, extracted with ethyl acetate three times and the combined organic layers were dried over anhydrous sodium sulfate, filtered, and concentrated under reduced pressure to afford a residue that was purified by preparative HPLC. The title compound was isolated in 29% yield. MS (ESI) m/z: 608.0 [M+H]⁺. ¹H NMR (400 MHz, acetonitrile-d₃) δ 8.74 (s, 1H), 7.96 (s, 1H), 7.92 (d, J=0.9 Hz, 1H), 7.30 (s, 1H), 6.98 (s, 1H), 4.75 (dd, J=7.8, 5.8 Hz, 2H), 4.39 (t, J=6.0 Hz, 2H), 3.92-3.83 (m, 2H), 3.76 (dd, J=6.5, 4.7 Hz, 2H), 3.54 (s, 2H), 3.13 (s, 3H), 2.84 (m, 4H), 2.69 (t, J=5.9 Hz, 2H), 2.23 (s, 3H).

### Example 217: 7-(2-((2-chloro-4-(4-(oxetan-3-ylmethyl)piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 216, where 4-methyl-7-(2-((7-methyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide was replaced with 7-(2-((2-chloro-4-(piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 66% yield. MS (ESI) m/z: 657.2 [M+H]⁺. ¹H NMR (400 MHz, acetonitrile-d₃) δ 8.75 (s, 1H), 8.00 (s, 1H), 7.92 (s, 1H), 7.59 (d, J=8.9 Hz, 1H), 7.06 (d, J=2.8 Hz, 1H), 6.96 (dd, J=9.0, 2.8 Hz, 1H), 4.73 (ddd, J=7.8, 5.9, 2.2 Hz, 2H), 4.35 (t, J=6.1 Hz, 2H), 3.88 (s, 2H), 3.77 (dd, J=6.5, 4.7 Hz, 2H), 3.28 (q, J=7.1 Hz, 1H), 3.19 (t, J=5.1 Hz, 4H), 3.14 (d, J=2.2 Hz, 3H), 2.74 (d, J=7.6 Hz, 2H), 2.54 (t, J=4.9 Hz, 4H).

### Example 218: 4-cyclobutyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(7-cyclopropyl-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclobutyl-3,4-dihydrothieno[2,3-f][1,4]thiazepin-5(2H)-one 1,1-dioxide in step 3. The title compound was isolated. MS (ESI) m/z: 604.2 [M+H]⁺. ¹H NMR (400 MHz, methanol-d₄) δ 8.72 (s, 1H), 7.99 (s, 1H), 7.42 (d, J=1.2 Hz, 1H), 6.78 (s, 1H), 4.76 (s, 1H), 3.98-3.93 (m, 4H), 3.79-3.74 (m, 2H), 3.10 (t, J=6.0 Hz, 2H), 2.86 (t, J=5.6 Hz, 2H), 2.32-2.25 (m, 4H), 1.95-1.87 (m, 1H), 1.85-1.74 (m, 2H), 0.93-0.87 (m, 2H), 0.63-0.58 (m, 2H).

### Example 219: 7-(2-((7-cyclopropyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(7-cyclopropyl-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-(oxetan-3-yl)-3,4-dihydrothieno[2,3-f][1,4]thiazepin-5(2H)-one 1,1-dioxide in step 3. The title compound was isolated. MS (ESI) m/z: 606.1 [M+H]⁺. ¹H NMR (400 MHz, DMSO-d₆) δ 9.93 (s, 1H), 8.82 (s, 1H), 7.82 (s, 1H), 7.20 (s, 1H), 6.76 (s, 1H), 5.27-5.18 (m, 1H), 4.77-4.65 (m, 4H), 4.06-3.96 (m, 6H), 3.18-3.12 (m, 3H), 2.84-2.78 (m, 2H), 2.00-1.92 (m, 1H), 0.86-0.80 (m, 2H), 0.60-0.51 (m, 2H).

### Example 220: 4-cyclopropyl-7-(2-((6-ethyl-1,2,3,4-tetrahydroisoquinolin-7-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(6-ethyl-7-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide in step 3. The title compound was isolated. MS (ESI) m/z: 578.3 [M+H]⁺. ¹H NMR (400 MHz, methanol-d₄) δ 8.69 (s, 1H), 7.97 (s, 1H), 7.27 (s, 1H), 7.12 (s, 1H), 4.13 (s, 2H), 3.98-3.95 (m, 2H), 3.76 (t, J=5.6 Hz, 2H), 3.27 (t, J=6.0 Hz, 2H), 2.96 (t, J=6.0 Hz, 2H), 2.90 (t, J=5.2 Hz, 1H), 2.65 (q, J=15.2, 7.6 Hz, 2H), 1.17 (t, J=7.2 Hz, 3H), 0.91-0.89 (m, 4H).

### Example 221: 4-cyclobutyl-7-(2-((6-ethyl-1,2,3,4-tetrahydroisoquinolin-7-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(6-ethyl-7-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclobutyl-3,4-dihydrothieno[2,3-f][1,4]thiazepin-5(2H)-one 1,1-dioxide in step 3. The title compound was isolated. MS (ESI) m/z: 592.3 [M+H]⁺. ¹H NMR (400 MHz, methanol-d₄) δ 8.71 (s, 1H), 7.98 (s, 1H), 7.32 (s, 1H), 7.15 (s, 1H), 4.75 (d, J=9.2 Hz, 1H), 4.20 (s, 2H), 3.96-3.94 (m, 2H), 3.77 (t, J=5.6 Hz, 2H), 3.37-3.34 (m, 2H), 3.01 (t, J=6.0 Hz, 2H), 2.66 (q, J=15.2, 7.6 Hz, 2H), 2.31-2.26 (m, 4H), 1.83-1.74 (m, 2H), 1.18 (t, J=7.6 Hz, 3H).

### Example 222: 7-(2-((2,7-diethyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-ethyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide and acetaldehyde. The title compound was isolated in 48% yield. m/z (ESI, +ve)=580.2 [M+H]⁺. ¹H NMR (400 MHz, methanol-d₄) δ=8.71 (s, 1H), 7.98 (s, 1H), 7.45-7.25 (m, 1H), 7.13 (s, 1H), 4.19-4.01 (m, 2H), 3.93 (d, J=5.0 Hz, 2H), 3.85 (d, J=5.5 Hz, 2H), 3.29-3.20 (m, 2H), 3.18 (s, 3H), 3.15-2.99 (m, 4H), 2.66 (q, J=7.6 Hz, 2H), 1.35 (t, J=7.3 Hz, 3H), 1.18 (t, J=7.6 Hz, 3H).

### Example 223: 7-(2-((2-(cyclopropylmethyl)-7-ethyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-ethyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide and cyclopropanecarbaldehyde. The title compound was isolated in 44% yield. m/z (ESI, +ve)=606.2 [M+H]⁺

¹H NMR (400 MHz, methanol-d₄) δ 8.72 (s, 1H), 7.99 (s, 1H), 7.48-7.31 (m, 1H), 7.12 (s, 1H), 4.12 (s, 2H), 3.98-3.92 (m, 2H), 3.88-3.81 (m, 2H), 3.24 (d, J=5.9 Hz, 2H), 3.19 (s, 3H), 3.10 (d, J=5.4 Hz, 2H), 2.82 (d, J=5.1 Hz, 2H), 2.67 (q, J=7.7 Hz, 2H), 1.18 (t, J=7.5 Hz, 3H), 1.16-1.09 (m, 1H), 0.72 (dd, J=1.3, 7.8 Hz, 2H), 0.37 (d, J=4.5 Hz, 2H).

### Example 224: 4-cyclopropyl-7-(2-((6-cyclopropyl-1,2,3,4-tetrahydroisoquinolin-7-yl)amino)-5-(trifluoromethyl)pyrimidin-4-yl)-3,4-dihydrothieno[2,3-f][1,4]thiazepin-5(2H)-one 1,1-dioxide

### Step 1: 1-(7-amino-6-bromo-3,4-dihydroisoquinolin-2(1H)-yl)-2,2,2-trifluoroethan-1-one

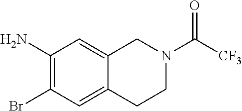 [SMILES: Nc1cc2c(cc1Br)CCN(C(=O)C(F)(F)F)C2]

To a solution of 1-(6-bromo-7-nitro-3,4-dihydroisoquinolin-2(1H)-yl)-2,2,2-trifluoroethan-1-one (20 mmol) in ethanol (60 mL) and water (30 mL) was added iron (99 mmol) and ammonium chloride (40 mmol). The mixture was stirred at 70° C. for 2 hours, cooled down to room temperature and filtered. Evaporation of volatiles afforded a crude product that was used into the next step without further purification. The title compound was isolated in 73% yield. ¹H NMR (400 MHz, CDCl₃) δ=7.26-7.20 (m, 1H), 6.58-6.51 (m, 1H), 4.67-4.54 (m, 2H), 3.88-3.79 (m, 2H), 2.86-2.80 (m, 2H).

### Step 2: 1-(7-amino-6-cyclopropyl-3,4-dihydroisoquinolin-2(1H)-yl)-2,2,2-trifluoroethan-1-one

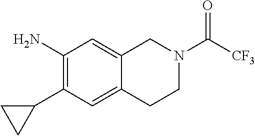 [SMILES: Nc1cc2c(cc1C1CC1)CCN(C(=O)C(F)(F)F)C2]

To a solution of 1-(7-amino-6-bromo-3,4-dihydroisoquinolin-2(1H)-yl)-2,2,2-trifluoroethan-1-one (14 mmol) and cyclopropylboronic acid (58 mmol) in toluene (100 mL) was added tricyclohexylphosphane (3 mmol), potassium phosphate (58 mmol) and diacetoxypalladium (1.5 mmol). The mixture was stirred at 110° C. for 12 hours, cooled down to room temperature and filtered. The filtrate was concentrated under reduced pressure and the crude was purified by silica gel chromatography (25% ethyl acetate in hexanes). The title compound was isolated in 54% yield. MS (ESI) m/z: 285.3 [M+H]⁺. ¹H NMR (400 MHz, DMSO-d₆) δ=6.65 (s, 1H), 6.45 (s, 1H), 4.73 (s, 2H), 4.56 (s, 2H), 3.74 (d, J=4.8 Hz, 2H), 2.77-2.70 (m, 2H), 1.67-1.59 (m, 1H), 0.88-0.83 (m, 2H), 0.44-0.40 (m, 2H).

### Step 3-4: 1-(6-cyclopropyl-7-((5-(trifluoromethyl)-4-(trimethylstannyl)pyrimidin-2-yl)amino)-3,4-dihydroisoquinolin-2(1H)-yl)-2,2,2-trifluoroethan-1-one

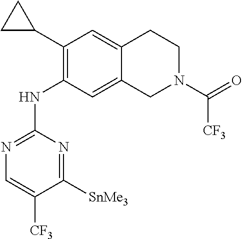

The title compound was prepared analogously to Example 212, steps 5-6, where 1-(7-amino-6-ethyl-3,4-dihydro-1H-isoquinolin-2-yl)-2,2,2-trifluoro-ethanone was replaced with 1-(7-amino-6-cyclopropyl-3,4-dihydroisoquinolin-2(1H)-yl)-2,2,2-trifluoroethan-1-one in step 4. The title compound was isolated in 31% yield. MS (ESI) m/z: 593.2 [M+H]⁺.

### Example 5-6: 4-cyclopropyl-7-(2-((6-cyclopropyl-1,2,3,4-tetrahydroisoquinolin-7-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(6-cyclopropyl-7-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-methyl-3,4-dihydrothieno[2,3-f][1,4]thiazepin-5(2H)-one 1,1-dioxide in step 3. The title compound was isolated. MS (ESI) m/z: 590.2 [M+H]⁺. ¹H NMR (400 MHz, methanol-d₄) δ=8.73 (s, 1H), 8.08 (s, 1H), 7.84 (s, 2H), 6.93 (s, 1H), 4.12-3.97 (m, 2H), 3.96-3.94 (m, 2H), 3.67-3.64 (m, 2H), 3.18-3.15 (m, 2H), 2.93-2.90 (m, 1H), 3.80-2.77 (m, 2H), 1.85-1.82 (m, 1H), 1.05-1.04 (m, 2H), 1.03-1.02 (m, 2H), 0.95-0.89 (m, 2H), 0.70-0.69 (m, 2H).

### Example 225: 4-cyclobutyl-7-(2-((6-cyclopropyl-1,2,3,4-tetrahydroisoquinolin-7-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(6-cyclopropyl-7-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclobutyl-3,4-dihydrothieno[2,3-f][1,4]thiazepin-5(2H)-one 1,1-dioxide in step 3. The title compound was isolated. MS (ESI) m/z: 604.2 [M+H]⁺. ¹H NMR (400 MHz, CDCl₃) δ=8.73 (s, 1H), 8.09 (s, 1H), 7.85 (d, J=5.6 Hz, 2H), 6.93 (s, 1H), 5.00-4.68 (m, 1H), 4.15 (s, 2H), 3.96-3.84 (m, 2H), 3.68 (t, J=5.6 Hz, 2H), 3.21 (t, J=5.6 Hz, 2H), 2.81 (t, J=5.6 Hz, 2H), 2.43-2.31 (m, 2H), 2.17-2.12 (m, 2H), 1.79 (s, 1H), 1.26 (s, 2H), 1.10-1.01 (m, 2H), 0.95-0.82 (m, 1H), 0.74-0.62 (m, 2H).

### Example 226: 4-cyclobutyl-7-(2-((7-cyclopropyl-2-ethyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclobutyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and acetaldehyde. The title compound was isolated in 24% yield. m/z (ESI, +ve)=623.3 [M+H]⁺. ¹H NMR (400 MHz, methanol-d₄) δ 8.73 (s, 1H), 8.00 (s, 1H), 7.51-7.43 (m, 1H), 6.83 (s, 1H), 4.75 (s, 1H), 3.99-3.92 (m, 2H), 3.77 (t, J=5.6 Hz, 2H), 3.65 (s, 2H), 2.97 (d, J=5.6 Hz, 2H), 2.82 (t, J=6.0 Hz, 2H), 2.65 (q, J=7.2 Hz, 2H), 2.33-2.26 (m, 4H), 1.97-1.87 (m, 1H), 1.85-1.75 (m, 2H), 1.22 (t, J=7.2 Hz, 3H), 0.94-0.88 (m, 2H), 0.65-0.60 (m, 2H).

### Example 227: 7-(2-((7-chloro-2-(2-fluoroethyl)-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide

To a solution of 7-(2-((7-chloro-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide (0.02 mmol) and 1-bromo-2-fluoroethane (0.02 mmol) in DMF (1 mL) was added potassium carbonate (0.03 mmol) and sodium iodide (0.002 mmol). The mixture was stirred at 60° C. for 8 hours, cooled down to room temperature and filtered. The filtrate was concentrated and the crude material purified by preparative HPLC. The title compound was isolated in 37% yield. MS (ESI) m/z: 630.2 [M+H]⁺. ¹H NMR (400 MHz, CDCl₃) δ=8.79 (s, 1H), 8.25 (s, 1H), 8.10 (s, 1H), 7.85 (s, 1H), 7.17 (s, 1H), 4.96-4.66 (m, 2H), 4.06-3.96 (m, 2H), 3.96-3.72 (m, 2H), 3.66 (t, J=6.0 Hz, 2H), 3.33-2.96 (m, 6H), 2.96-2.89 (m, 1H), 1.00-0.93 (m, 4H).

### Example 228: 4-cyclopropyl-7-(2-((2,7-diethyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((7-ethyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and acetaldehyde. The title compound was isolated in 17% yield. m/z (ESI, +ve)=606.3 [M+H]⁺. ¹H NMR (400 MHz, methanol-d₄) δ=8.71 (s, 1H), 7.97 (s, 1H), 7.37 (d, J=0.8 Hz, 1H), 7.11 (s, 1H), 4.02 (s, 2H), 3.96 (t, J=6.0 Hz, 2H), 3.78-3.74 (m, 2H), 3.16 (d, J=4.8 Hz, 2H), 3.09 (d, J=4.8 Hz, 2H), 2.97 (d, J=7.2 Hz, 2H), 2.89 (d, J=5.6 Hz, 1H), 2.69-2.63 (m, 2H), 1.33 (t, J=7.2 Hz, 3H), 1.18 (t, J=7.2 Hz, 3H), 0.92-0.88 (m, 4H).

### Example 229: 7-(2-((7-ethyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(7-ethyl-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-(oxetan-3-yl)-3,4-dihydrothieno[2,3-f][1,4]thiazepin-5(2H)-one 1,1-dioxide in step 3. The title compound was isolated. MS (ESI) m/z: 557.9 [M+1]⁺. ¹H NMR (400 MHz, DMSO-d₆) δ 9.87 (s, 1H), 8.80 (br s, 1H), 7.83 (s, 1H), 7.12 (br s, 1H), 6.98 (s, 1H), 5.28-5.17 (m, 1H), 4.77-4.72 (m, 2H), 4.71-4.65 (m, 2H), 4.06-3.97 (m, 4H), 3.96-3.93 (m, 2H), 3.18-3.15 (m, 2H), 3.09-3.03 (m, 2H), 2.77-2.69 (m, 2H), 2.58-2.54 (m, 1H), 1.07 (t, J=7.6 Hz, 3H).

### Example 230: 7-(2-((6-chloro-1,2,3,4-tetrahydroisoquinolin-7-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(6-chloro-7-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-(oxetan-3-yl)-3,4-dihydrothieno[2,3-f][1,4]thiazepin-5(2H)-one 1,1-dioxide in step 3. The title compound was isolated. MS (ESI) m/z: 600.1 [M+H]⁺. ¹H NMR (400 MHz, DMSO-d₆) δ 10.08 (s, 1H), 8.86 (s, 1H), 7.84 (s, 1H), 7.32-7.21 (m, 2H), 5.26-5.18 (m, 1H), 4.76-4.67 (m, 4H), 4.05-3.97 (m, 4H), 3.89-3.83 (m, 2H), 3.01-2.94 (m, 2H), 2.76-2.68 (m, 2H).

### Example 231: 7-(2-((6-cyclopropyl-1,2,3,4-tetrahydroisoquinolin-7-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(6-cyclopropyl-7-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-methyl-3,4-dihydrothieno[2,3-f][1,4]thiazepin-5(2H)-one 1,1-dioxide in step 3. The title compound was isolated. MS (ESI) m/z: 564.1 [M+H]⁺. ¹H NMR (400 MHz, methanol-d₄) δ=8.72 (s, 1H), 8.00 (s, 1H), 7.48-7.34 (m, 1H), 6.85 (s, 1H), 4.64-4.55 (m, 1H), 4.02 (s, 2H), 3.97-3.92 (m, 2H), 3.87-3.83 (m, 2H), 3.18 (s, 3H), 3.13 (t, J=6.0 Hz, 2H), 2.83 (t, J=6.0 Hz, 2H), 1.97-1.89 (m, 1H), 0.95-0.89 (m, 2H), 0.65-0.59 (m, 2H).

### Example 232: 7-(2-((6-cyclopropyl-1,2,3,4-tetrahydroisoquinolin-7-yl)amino)-5-(trifluoromethyl)pyrimidin-4-yl)-4-(2,2,2-trifluoroethyl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(6-cyclopropyl-7-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-(2,2,2-trifluoroethyl)-3,4-dihydrothieno[2,3-f][1,4]thiazepin-5(2H)-one 1,1-dioxide in step 3. The title compound was isolated. MS (ESI) m/z: 632.1 [M+H]⁺. ¹H NMR (400 MHz, MeOD) δ 8.74 (s, 1H), 8.02 (s, 1H), 7.41-7.28 (m, 1H), 6.85 (s, 1H), 4.37 (q, J=9.2 Hz, 2H), 4.09 (d, J=5.2 Hz, 2H), 4.00 (s, 2H), 3.84 (t, J=5.6 Hz, 2H), 3.11 (t, J=6.0 Hz, 2H), 2.82 (t, J=5.6 Hz, 2H), 1.94-1.90 (m, 1H), 0.93-0.90 (m, 2H), 0.63-0.60 (m, 2H).

### Example 233: 4-cyclopropyl-7-(2-((7-cyclopropyl-2-ethyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and acetaldehyde. The title compound was isolated in 69% yield. m/z (ESI, +ve)=618.3 [M+H]⁺. ¹H NMR (400 MHz, methanol-d₄) δ 8.74 (s, 1H), 7.99 (s, 1H), 7.54 (s, 1H), 6.87 (s, 1H), 3.97 (t, J=5.6 Hz, 2H), 3.83 (s, 2H), 3.80-3.75 (m, 2H), 3.02 (dd, J=4.4, 10.0 Hz, 4H), 2.90 (td, J=2.8, 5.2 Hz, 1H), 2.82 (q, J=7.2 Hz, 2H), 1.95-1.88 (m, 1H), 1.29-1.24 (m, 3H), 0.95-0.89 (m, 6H), 0.66-0.60 (m, 2H).

### Example 234: 4-cyclopropyl-7-(2-((7-cyclopropyl-2-(oxetan-3-ylmethyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and oxetane-3-carbaldehyde. The title compound was isolated in 42% yield. m/z (ESI, +ve)=660.3 [M+H]⁺. ¹H NMR (400 MHz, methanol-d₄) δ 8.77 (s, 1H), 8.00 (s, 1H), 7.66 (s, 1H), 6.94 (s, 1H), 4.92 (s, 2H), 4.56 (t, J=6.4 Hz, 2H), 4.21 (s, 1H), 4.01-3.94 (m, 2H), 3.82-3.73 (m, 3H), 3.67-3.59 (m, 1H), 3.54 (d, J=6.4 Hz, 2H), 3.42 (d, J=2.0 Hz, 2H), 3.19 (t, J=5.6 Hz, 2H), 2.95-2.87 (m, 1H), 2.03-1.93 (m, 1H), 1.01-0.95 (m, 2H), 0.94-0.90 (m, 4H), 0.68-0.62 (m, 2H).

### Example 235: 4-cyclopropyl-7-(2-((7-cyclopropyl-2-(cyclopropylmethyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and cyclopropanecarbaldehyde. The title compound was isolated in 99% yield. m/z (ESI, +ve)=644.3 [M+H]⁺. ¹H NMR (400 MHz, methanol-d₄) δ 8.76 (s, 1H), 8.00 (s, 1H), 7.61 (s, 1H), 6.92 (s, 1H), 4.59 (s, 2H), 4.12 (s, 2H), 3.98 (t, J=5.6 Hz, 2H), 3.81-3.74 (m, 2H), 3.18-3.09 (m, 2H), 2.94-2.83 (m, 3H), 1.97-1.94 (m, 1H), 1.18-1.09 (m, 1H), 0.98-0.90 (m, 6H), 0.77-0.72 (m, 2H), 0.67-0.62 (m, 2H), 0.43-0.35 (m, 2H).

### Example 236: 7-(2-((7-cyclopropyl-2-(2-fluoroethyl)-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 227, where 7-(2-((7-chloro-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((7-cyclopropyl-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 41% yield. MS (ESI) m/z: 610.2 [M+H]⁺. ¹H NMR (400 MHz, methanol-d₄) δ=8.85 (s, 1H), 8.12 (s, 1H), 7.59 (d, J=1.6 Hz, 1H), 6.93 (s, 1H), 4.87-4.85 (m, 1H), 4.75-4.73 (m, 1H), 4.13-4.03 (m, 2H), 3.99-3.93 (m, 2H), 3.85 (s, 2H), 3.31 (s, 3H), 3.15-2.98 (m, 6H), 2.09-2.02 (m, 1H), 1.05-1.02 (m, 2H), 0.74 (dd, J=1.6, 5.2 Hz, 2H).

### Example 237: 7-(2-((7-chloro-2-(2-fluoroethyl)-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 227, where 7-(2-((7-chloro-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((7-chloro-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 34% yield. MS (ESI) m/z: 604.2 [M+H]⁺. ¹H NMR (400 MHz, methanol-d₄) δ=8.79 (s, 1H), 8.02 (s, 1H), 7.71 (s, 1H), 7.24 (s, 1H), 4.74 (s, 1H), 4.61 (d, J=4.8 Hz, 1H), 4.58 (s, 2H), 3.94 (d, J=5.6 Hz, 2H), 3.86 (d, J=5.2 Hz, 2H), 3.75 (s, 2H), 3.19 (s, 3H), 2.87 (d, J=4.8 Hz, 4H).

### Example 238: 7-(2-((2-cyclopropyl-4-(piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

### Step 1: tert-butyl 4-(3-bromo-4-nitrophenyl)piperazine-1-carboxylate

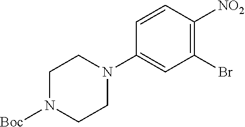 [SMILES: CC(C)(C)OC(=O)N1CCN(c2ccc([N+](=O)[O-])c(Br)c2)CC1]

A solution of 2-bromo-4-fluoro-1-nitro-benzene (86 mmol), tert-butyl piperazine-1-carboxylate (95 mmol) and potassium carbonate (173 mmol) in DMF (190 mL) was stirred at 60° C. for 12 hours. The mixture was cooled down to room temperature and poured over ice-water, which resulted in formation of yellow precipitate. The precipitate was filtered and dried under vacuum. The title compound was isolated in 90% yield. ¹H NMR (400 MHz, CDCl₃) δ 8.01 (d, J=9.2 Hz, 1H), 7.06 (d, J=2.4 Hz, 1H), 6.76 (dd, J=9.2, 2.4 Hz, 1H), 3.61-3.59 (m, 4H), 3.40-3.38 (m, 4H), 1.49 (s, 9H).

### Step 2: tert-butyl 4-(3-cyclopropyl-4-nitrophenyl)piperazine-1-carboxylate

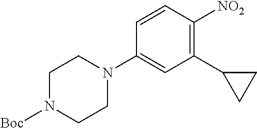 [SMILES: CC(C)(C)OC(=O)N1CCN(c2ccc([N+](=O)[O-])c(C3CC3)c2)CC1]

The title compound was prepared analogously to Example 224, step 2, where 1-(7-amino-6-bromo-3,4-dihydroisoquinolin-2(1H)-yl)-2,2,2-trifluoroethan-1-one was replaced with tert-butyl 4-(3-bromo-4-nitrophenyl)piperazine-1-carboxylate. The title compound was isolated in 56% yield. ¹H NMR (400 MHz, CDCl₃) δ 7.99 (d, J=9.2 Hz, 1H), 6.68 (dd, J=9.2, 2.8 Hz, 1H), 6.54 (d, J=2.8 Hz, 1H), 3.60-3.58 (m, 4H), 3.36-3.33 (m, 4H), 2.64-2.58 (m, 1H), 1.06-1.04 (m, 2H), 0.69-0.66 (m, 2H).

### Step 3: tert-butyl 4-(4-amino-3-cyclopropylphenyl)piperazine-1-carboxylate

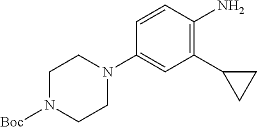 [SMILES: CC(C)(C)OC(=O)N1CCN(c2ccc(N)c(C3CC3)c2)CC1]

The title compound was prepared analogously to Example 224, step 1 where 1-(6-bromo-7-nitro-3,4-dihydroisoquinolin-2(1H)-yl)-2,2,2-trifluoroethan-1-one was replaced with tert-butyl 4-(3-cyclopropyl-4-nitrophenyl)piperazine-1-carboxylate. The title compound was isolated in 71% yield. MS (ESI) m/z: 318.2 [M+H]⁺.

### Step 4: tert-butyl 4-(4-((4-chloro-5-(trifluoromethyl)pyrimidin-2-yl)amino)-3-cyclopropylphenyl)piperazine-1-carboxylate

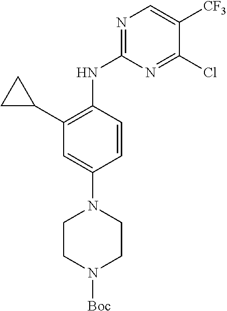 [SMILES: CC(C)(C)OC(=O)N1CCN(c2ccc(Nc3ncc(C(F)(F)F)c(Cl)n3)c(C3CC3)c2)CC1]

The title compound was prepared analogously to Example 121, step 3, where tert-butyl 3-(3-amino-4-chlorophenyl)azetidine-1-carboxylate was replaced with tert-butyl 4-(4-amino-3-cyclopropylphenyl)piperazine-1-carboxylate. The title compound was isolated. MS (ESI) m/z: 498.3 [M+H]⁺. ¹H NMR (400 MHz, CDCl₃) δ 8.53 (s, 1H), 7.91-7.72 (m, 1H), 7.65-7.48 (m, 1H), 6.93-6.66 (m, 2H), 3.59 (s, 4H), 3.12 (s, 4H), 1.92-1.77 (m, 1H), 1.02 (d, J=7.6 Hz, 2H), 0.69 (d, J=4.4 Hz, 2H).

### Step 5: tert-butyl 4-(3-cyclopropyl-4-((5-(trifluoromethyl)-4-(trimethylstannyl)pyrimidin-2-yl)amino)phenyl)piperazine-1-carboxylate

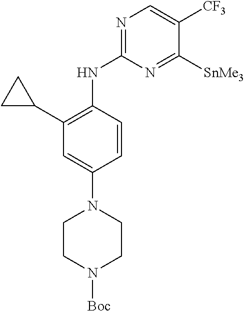

The title compound was prepared analogously to Example 80, step 3 where 1-(5-((4-chloro-5-(trifluoromethyl)pyrimidin-2-yl)amino)-6-fluoroisoindolin-2-yl)-2,2,2-trifluoroethan-1-one was replaced with tert-butyl 4-(4-((4-chloro-5-(trifluoromethyl)pyrimidin-2-yl)amino)-3-cyclopropylphenyl)piperazine-1-carboxylate. The title compound was isolated in 66% yield. MS (ESI) m/z: 628.3 [M+H]⁺.

### Step 6: 7-(2-((2-cyclopropyl-4-(piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 149, steps 5-6, where tert-butyl 4-(4-chloro-3-((5-(trifluoromethyl)-4-(trimethylstannyl)pyrimidin-2-yl)amino)phenyl)piperidine-1-carboxylate and 7-bromo-4-methyl-3,4-dihydrothieno[2,3-f][1,4]thiazepin-5(2H)-one 1,1-dioxide were replaced with tert-butyl 4-(3-cyclopropyl-4-((5-(trifluoromethyl)-4-(trimethylstannyl)pyrimidin-2-yl)amino)phenyl)piperazine-1-carboxylate and 7-bromo-4-methyl-3,4-dihydrothieno[2,3-f][1,4]thiazepin-5(2H)-one 1,1-dioxide in step 5. The title compound was isolated. MS (ESI) m/z: 593.1 [M+H]⁺. ¹H NMR (400 MHz, MeOD) δ 8.75-8.60 (m, 1H), 7.98 (s, 1H), 7.47-7.27 (m, 1H), 6.87 (dd, J=8.8, 2.4 Hz, 1H), 6.66 (d, J=2.4 Hz, 1H), 3.92 (s, 2H), 3.18 (s, 3H), 3.15-3.10 (m, 4H), 3.00-2.95 (m, 4H), 2.00-1.90 (m, 1H), 0.93-0.86 (m, 3H), 0.68-0.61 (m, 2H).

### Example 239: 4-cyclopropyl-7-(2-((2-cyclopropyl-4-(piperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 238, where 7-bromo-4-methyl-3,4-dihydrothieno[2,3-f][1,4]thiazepin-5(2H)-one 1,1-dioxide was replaced with tert-butyl 4-(3-cyclopropyl-4-((5-(trifluoromethyl)-4-(trimethylstannyl)pyrimidin-2-yl)amino)phenyl)piperazine-1-carboxylate in step 5. The title compound was isolated in 43% yield.

MS (ESI) m/z: 619.2 [M+H]⁺. ¹H NMR (400 MHz, CDCl₃) δ 8.69 (s, 1H), 8.05 (s, 1H), 7.90-7.88 (m, 1H), 7.68-7.65 (m, 1H), 6.90-6.88 (m, 1H), 6.76 (s, 1H), 3.93 (d, J=6.0 Hz, 2H), 3.64 (t, J=5.6 Hz, 2H), 3.14 (d, J=4.4 Hz, 4H), 3.06 (d, J=5.2 Hz, 4H), 2.93-2.89 (m, 1H), 1.92-1.85 (m, 1H), 1.03-1.01 (m, 2H), 0.97-0.94 (m, 4H), 0.72-0.71 (m, 2H).

### Example 240: 4-cyclopropyl-7-(2-((2-cyclopropyl-4-(piperidin-4-yl)phenyl)amino)-5-(trifluoromethyl)pyrimidin-4-yl)-3,4-dihydrothieno[2,3-f][1,4]thiazepin-5(2H)-one 1,1-dioxide

### Step 1: tert-butyl 4-(4-amino-3-cyclopropylphenyl)piperidine-1-carboxylate

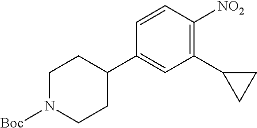 [SMILES: CC(C)(C)OC(=O)N1CCC(c2ccc([N+](=O)[O-])c(C3CC3)c2)CC1]

The title compound was prepared analogously to Example 224, step 2 where 1-(7-amino-6-bromo-3,4-dihydroisoquinolin-2(1H)-yl)-2,2,2-trifluoroethan-1-one was replaced with tert-butyl 4-(4-amino-3-bromophenyl)piperidine-1-carboxylate. The title compound was isolated in 71% yield.. MS (ESI) m/z: 261.3 [M−55]⁺.

### Step 2: tert-butyl 4-(4-((4-chloro-5-(trifluoromethyl)pyrimidin-2-yl)amino)-3-cyclopropylphenyl)piperidine-1-carboxylate

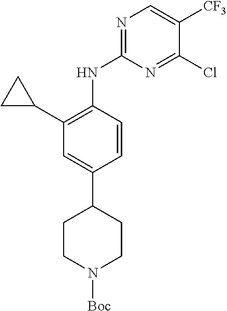 [SMILES: CC(C)(C)OC(=O)N1CCC(c2ccc(Nc3ncc(C(F)(F)F)c(Cl)n3)c(C3CC3)c2)CC1]

The title compound was prepared analogously to Example 121, step 3, where tert-butyl 3-(3-amino-4-chlorophenyl)azetidine-1-carboxylate was replaced with tert-butyl 4-(4-amino-3-cyclopropylphenyl)piperidine-1-carboxylate. The title compound was isolated in 24% yield. ¹H NMR (400 MHz, CDCl₃) δ 8.56 (s, 1H), 7.99 (d, J=8.4 Hz, 1H), 7.79 (s, 1H), 7.13 (dd, J=1.6, 8.4 Hz, 1H), 7.02 (d, J=1.6 Hz, 1H), 4.25 (d, J=12.4 Hz, 2H), 2.80 (t, J=12.4 Hz, 2H), 2.67-2.58 (m, 1H), 1.86-1.79 (m, 3H), 1.68-1.60 (m, 2H), 1.50 (s, 9H), 1.07-1.02 (m, 2H), 0.73-0.68 (m, 2H).

### Step 3: tert-butyl 4-(3-cyclopropyl-4-((5-(trifluoromethyl)-4-(trimethylstannyl)pyrimidin-2-yl)amino)phenyl)piperidine-1-carboxylate

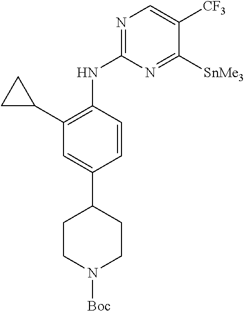

The title compound was prepared analogously to Example 80, step 3 where 1-(5-((4-chloro-5-(trifluoromethyl)pyrimidin-2-yl)amino)-6-fluoroisoindolin-2-yl)-2,2,2-trifluoroethan-1-one was replaced with tert-butyl 4-(4-((4-chloro-5-(trifluoromethyl)pyrimidin-2-yl)amino)-3-cyclopropylphenyl)piperidine-1-carboxylate. The title compound was isolated in 41% yield as a colorless oil. MS (ESI) m/z: 627.3 [M+H]⁺.

### Step 4-5: 4-cyclopropyl-7-(2-((2-cyclopropyl-4-(piperidin-4-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 238, steps 6-7, where tert-butyl 4-(3-cyclopropyl-4-((5-(trifluoromethyl)-4-(trimethylstannyl)pyrimidin-2-yl)amino)phenyl)piperazine-1-carboxylate and 7-bromo-4-methyl-3,4-dihydrothieno[2,3-f][1,4]thiazepin-5(2H)-one 1,1-dioxide were replaced with tert-butyl 4-(3-cyclopropyl-4-((5-(trifluoromethyl)-4-(trimethylstannyl)pyrimidin-2-yl)amino)phenyl)piperidine-1-carboxylate and 7-bromo-4-cyclopropyl-3,4-dihydrothieno[2,3-f][1,4]thiazepin-5(2H)-one 1,1-dioxide in step 6. The title compound was isolated. MS (ESI) m/z: 618.2 [M+H]⁺. ¹H NMR (400 MHz, CDCl₃) δ 8.75 (s, 1H), 8.11-8.06 (m, 2H), 7.88 (s, 1H), 7.20 (d, J=8.0 Hz, 1H), 7.08 (s, 1H), 3.96 (t, J=6.0 Hz, 2H), 3.66 (t, J=6.0 Hz, 2H), 3.59-3.52 (m, 2H), 3.04 (dt, J=4.0, 12.4 Hz, 2H), 2.94-2.90 (m, 1H), 2.78-2.72 (m, 1H), 2.10-2.04 (m, 4H), 1.88 (d, J=5.2 Hz, 1H), 1.10-1.06 (m, 2H), 0.98-0.92 (m, 4H), 0.74-0.70 (m, 2H).

### Example 241: 4-cyclopropyl-7-(2-((2-cyclopropyl-4-(1-(cyclopropylmethyl)piperidin-4-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((2-cyclopropyl-4-(piperidin-4-yl)phenyl)amino)-5-(trifluoromethyl)pyrimidin-4-yl)-3,4-dihydrothieno[2,3-f][1,4]thiazepin-5(2H)-one 1,1-dioxide and cyclopropanecarbaldehyde. The title compound was isolated in 57% yield. m/z (ESI, +ve)=672.2 [M+H]⁺

¹H NMR (400 MHz, CDCl₃) δ 8.73 (s, 1H), 8.07-8.03 (m, 2H), 7.85 (s, 1H), 7.20 (d, J=8.4 Hz, 1H), 7.09 (s, 1H), 3.96 (t, J=6.0 Hz, 2H), 3.65 (t, J=6.0 Hz, 2H), 3.45-3.36 (m, 2H), 2.96-2.90 (m, 1H), 2.62-2.48 (m, 3H), 2.40-2.28 (m, 2H), 2.08-1.97 (m, 3H), 1.91 (s, 2H), 1.08-1.02 (m, 3H), 0.98-0.93 (m, 4H), 0.72 (d, J=5.2 Hz, 2H), 0.64 (d, J=7.6 Hz, 2H), 0.25 (d, J=5.2 Hz, 2H).

### Example 242: 4-cyclopropyl-7-(2-((2-(cyclopropylmethyl)-7-ethyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((7-ethyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and cyclopropanecarbaldehyde. The title compound was isolated in 47% yield. m/z (ESI, +ve)=632.2 [M+H]⁺. ¹H NMR (400 MHz, methanol-d₄) δ=8.72 (s, 1H), 7.98 (s, 1H), 7.43-7.33 (m, 1H), 7.12 (s, 1H), 4.59 (s, 2H), 4.15-4.07 (m, 2H), 3.96 (s, 2H), 3.81-3.73 (m, 2H), 3.26 (s, 2H), 3.10 (s, 2H), 2.90 (s, 1H), 2.83 (d, J=6.4 Hz, 2H), 2.67 (d, J=7.6 Hz, 2H), 1.29 (s, 2H), 1.19 (t, J=7.2 Hz, 3H), 1.14-1.08 (m, 1H), 0.91 (s, 2H), 0.72 (d, J=6.8 Hz, 2H), 0.39-0.35 (m, 2H).

### Example 243: 7-(2-((7-chloro-2-(2-fluoroethyl)-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 227, where 7-(2-((7-chloro-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 4-cyclopropyl-7-(2-((7-ethyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide. The title compound was isolated in 39% yield. MS (ESI) m/z: 624.2 [M+H]⁺. ¹H NMR (400 MHz, methanol-d₄) δ 8.69 (s, 1H), 7.97 (s, 1H), 7.26 (s, 1H), 7.03 (s, 1H), 4.75 (t, J=4.8 Hz, 1H), 4.63 (t, J=4.8 Hz, 1H), 3.95 (t, J=5.6 Hz, 2H), 3.79 (s, 2H), 3.75 (t, J=6.0 Hz, 2H), 2.99-2.96 (m, 3H), 2.93-2.89 (m, 4H), 2.63 (q, J=15.2, 7.6 Hz, 2H), 1.17 (t, J=7.6 Hz, 3H), 0.91-0.89 (m, 4H).

### Example 244: 7-(2-((7-cyclopropyl-2-ethyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-cyclopropyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide and acetaldehyde. The title compound was isolated in 82% yield. m/z (ESI, +ve)=634.2 [M+H]⁺. ¹H NMR (400 MHz, DMSO-d₆) δ 9.88 (s, 1H), 8.82 (br s, 1H), 7.83 (s, 1H), 7.16 (br s, 1H), 6.70 (s, 1H), 5.27-5.18 (m, 1H), 4.76-4.67 (m, 4H), 4.05-3.96 (m, 4H), 3.61-3.51 (m, 2H), 2.83-2.75 (m, 2H), 2.70-2.66 (m, 1H), 2.59-2.54 (m, 1H), 1.92-1.89 (m, 3H), 1.11 (t, J=6.8 Hz, 3H), 0.83-0.78 (m, 2H), 0.58-0.53 (m, 2H).

### Example 245: 7-(2-((7-cyclopropyl-2-ethyl-1,2,3,4-tetrahydroisoquinolin-6-yl)amino)-5-(trifluoromethyl)pyrimidin-4-yl)-4-(2,2,2-trifluoroethyl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-cyclopropyl-1,2,3,4-tetrahydroisoquinolin-6-yl)amino)-5-(trifluoromethyl)pyrimidin-4-yl)-4-(2,2,2-trifluoroethyl)-3,4-dihydrothieno[2,3-f][1,4]thiazepin-5(2H)-one 1,1-dioxide and acetaldehyde. The title compound was isolated. m/z (ESI, +ve)=661.1 [M+H]⁺. ¹H NMR (400 MHz, DMSO-d₆) δ 9.90 (s, 1H), 8.83 (s, 1H), 7.84 (s, 1H), 7.16 (s, 1H), 6.70 (s, 1H), 4.44 (d, J=9.5 Hz, 2H), 4.06 (s, 2H), 3.93 (s, 2H), 3.50 (s, 2H), 2.78 (s, 2H), 2.63 (s, 2H), 1.93 (d, J=9.0 Hz, 2H), 1.09 (t, J=7.1 Hz, 3H), 0.88-0.79 (m, 2H), 0.61-0.54 (m, 2H).

### Example 246: 7-(2-((7-chloro-2-(cyclopropylmethyl)-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-chloro-1,2,3,4-tetrahydroisoquinolin-6-yl)amino)-5-(trifluoromethyl)pyrimidin-4-yl)-4-methyl-3,4-dihydrothieno[2,3-f][1,4]thiazepin-5(2H)-one 1,1-dioxide and cyclopropanecarbaldehyde. The title compound was isolated in 10% yield. m/z (ESI, +ve)=612.1 [M+H]⁺. ¹H NMR (400 MHz, DMSO-d₆) δ 10.06 (s, 1H), 8.87 (s, 1H), 7.83 (s, 1H), 7.35 (s, 1H), 7.30 (s, 1H), 3.96 (d, J=6.0 Hz, 2H), 3.89 (d, J=6.3 Hz, 2H), 3.64 (s, 2H), 3.08 (s, 3H), 2.82 (d, J=5.7 Hz, 2H), 2.73 (t, J=5.7 Hz, 2H), 2.37 (d, J=6.6 Hz, 2H), 0.93 (d, J=5.2 Hz, 1H), 0.56-0.48 (m, 2H), 0.20-0.10 (m, 2H).

### Example 247: 4-cyclopropyl-7-(2-((7-(methylthio)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

### Step 1: 4-cyclopropyl-7-(trimethylstannyl)-3,4-dihydrothieno[2,3-f][1,4]thiazepin-5(2H)-one 1,1-dioxide

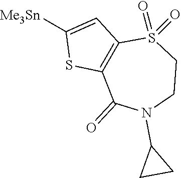

A solution of 7-bromo-4-cyclopropyl-2H,3H,4H,5H-1λ⁶-thieno[2,3-f][1,4]thiazepine-1,1,5-trione (3.1 mmol) in dioxane (21 ml) was treated with hexamethylditin (6.2 mmol) and tetrakis(triphenylphosphine)palladium (0.16 mmol). The reaction mixture was stirred overnight at 95° C., cooled down to room temperature and filtered through celite. The volatiles were removed under reduced pressure and the residue purified by silica gel chromatography (0-30% of ethyl acetate in dichloromethane) to afford the title compound in 74% yield. m/z (ESI, +ve)=421.9 [M+H]⁺.

### Step 2: 4-Cyclopropyl-7-(2-((7-(methylthio)-2-(2,2,2-trifluoroacetyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

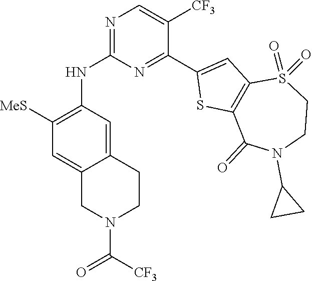 [SMILES: CSc1cc2c(cc1Nc1ncc(C(F)(F)F)c(-c3cc4c(s3)C(=O)N(C3CC3)CCS4(=O)=O)n1)CCN(C(=O)C(F)(F)F)C2]

The title compound was prepared analogously to Example 155, step 3, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one was replaced with 1-(6-{[4-chloro-5-(trifluoromethyl)pyrimidin-2-yl]amino}-7-(methylsulfanyl)-1,2,3,4-tetrahydroisoquinolin-2-yl)-2,2,2-trifluoroethan-1-one in step 3. The title compound was isolated in 94% yield. MS (ESI) m/z: 692.2 [M+H]⁺.

### Step 3: 4-cyclopropyl-7-(2-((7-(methylthio)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

A solution of 4-cyclopropyl-7-(2-{[7-(methylsulfanyl)-2-(2,2,2-trifluoroacetyl)-1,2,3,4-tetrahydroisoquinolin-6-yl]amino}-5-(trifluoromethyl)pyrimidin-4-yl)-2H,3H,4H,5H-1λ⁶-thieno[2,3-f][1,4]thiazepine-1,1,5-trione (0.44 mmol) in dichloromethane (3 mL), was treated with a 7M solution of ammonia in methanol (6.3 mL). After 1 hour at room temperature, the reaction mixture was concentrated and the residue was purified by silica gel chromatography (0-20% of 0.7M NH3 in MeOH in DCM). The title compound was isolated in 70% yield. MS (ESI) m/z: 696.2 [M+H]⁺. ¹H NMR (400 MHz, DMSO-d₆) δ 2.15 (s, 3H), 2.66 (m, 2H), 2.93 (t, J=5.8 Hz, 2H), 3.07 (s, 3H), 3.81 (s, 2H), 3.89 (m, 2H), 3.96 (m, 2H), 6.91 (s, 1H), 7.09 (s, 1H), 7.81 (s, 1H), 8.81 (s, 1H), 9.82 (s, 1H).

### Example 248: 4-cyclopropyl-7-(2-((7-cyclopropyl-2-isopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and acetone. The title compound was isolated in 86% yield. m/z (ESI, +ve)=632.1 [M+H]⁺. ¹H NMR (400 MHz, methanol-d₄) δ 8.76 (s, 1H), 7.99 (s, 1H), 7.60 (s, 1H), 6.93 (s, 1H), 4.12 (s, 2H), 3.97 (t, J=5.6 Hz, 2H), 3.80-3.74 (m, 2H), 3.41-3.34 (m, 1H), 3.28-3.26 (m, 1H), 3.17-3.09 (m, 2H), 2.94-2.87 (m, 1H), 2.00-1.90 (m, 2H), 1.34 (d, J=6.4 Hz, 6H), 0.98-0.93 (m, 2H), 0.92-0.89 (m, 4H), 0.67-0.61 (m, 2H).

### Example 249: 4-cyclopropyl-7-(2-((7-cyclopropyl-2-(2-fluoroethyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 227, where 7-(2-((7-chloro-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 4-cyclopropyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide. The title compound was isolated in 51% yield. MS (ESI) m/z: 636.2[M+H]⁺. ¹H NMR (400 MHz, methanol-d₄) δ 8.75 (s, 1H), 7.99 (s, 1H), 7.57 (s, 1H), 6.88 (s, 1H), 4.92 (d, J=3.2 Hz, 1H), 4.73 (t, J=4.4 Hz, 1H), 4.04 (s, 2H), 4.01-3.95 (m, 2H), 3.77 (t, J=5.6 Hz, 2H), 3.26-3.16 (m, 4H), 3.10 (d, J=5.6 Hz, 2H), 2.95-2.87 (m, 1H), 1.99-1.89 (m, 1H), 0.95-0.88 (m, 6H), 0.64 (d, J=4.0 Hz, 2H).

### Example 250: 7-(2-((2-cyclopropyl-4-(4-(cyclopropylmethyl)piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((2-cyclopropyl-4-(piperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide and cyclopropanecarbaldehyde. The title compound was isolated in 96% yield. m/z (ESI, +ve)=647.2 [M+H]⁺. ¹H NMR (400 MHz, MeOD, 298 K) δ 8.70 (s, 1H), 8.07 (s, 1H), 7.91-7.87 (m, 1H), 7.72-6.90 (m, 1H), 6.89 (d, J=2.8 Hz, 1H), 6.74 (d, J=2.8 Hz, 1H), 3.99-3.85 (m, 2H), 3.80-3.68 (m, 2H), 3.44-3.32 (m, 4H), 3.25 (s, 3H), 3.09 (s, 4H), 2.67 (d, J=7.2 Hz, 2H), 1.95-1.82 (m, 1H), 1.26 (s, 2H), 1.09-0.98 (m, 2H), 0.96-0.79 (m, 1H), 0.75-0.60 (m, 4H), 0.29 (d, J=5.2 Hz, 2H).

### Example 251: 4-cyclopropyl-7-(2-((2-cyclopropyl-4-(4-(cyclopropylmethyl)piperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((2-cyclopropyl-4-(piperazin-1-yl)phenyl)amino)-5-(trifluoromethyl)pyrimidin-4-yl)-3,4-dihydrothieno[2,3-f][1,4]thiazepin-5(2H)-one 1,1-dioxide and cyclopropanecarbaldehyde. The title compound was isolated in 57% yield. m/z (ESI, +ve)=719.3 [M+H]⁺.

### Example 252: 4-cyclopropyl-7-(2-((2-cyclopropyl-4-(1-methylpiperidin-4-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide was replaced with 4-cyclopropyl-7-(2-((2-cyclopropyl-4-(piperidin-4-yl)phenyl)amino)-5-(trifluoromethyl)pyrimidin-4-yl)-3,4-dihydrothieno[2,3-f][1,4]thiazepin-5(2H)-one 1,1-dioxide. The title compound was isolated in 59% yield. m/z (ESI, +ve)=632.2 [M+H]⁺. ¹H NMR (400 MHz, CDCl₃) δ 8.74 (s, 1H), 8.12-7.96 (m, 2H), 7.87 (s, 1H), 7.19 (d, J=8.8 Hz, 1H), 7.07 (s, 1H), 3.96 (t, J=6.0 Hz, 2H), 3.66 (t, J=6.0 Hz, 2H), 3.57-3.42 (m, 2H), 2.98-2.88 (m, 1H), 2.74 (s, 3H), 2.68-2.56 (m, 2H), 2.21-2.08 (m, 3H), 2.01 (s, 3H), 1.26 (s, 2H), 1.09-0.95 (m, 4H), 0.75-0.70 (m, 2H).

### Example 253: 4-cyclopropyl-7-(2-((2-cyclopropyl-4-(1-ethylpiperidin-4-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((2-cyclopropyl-4-(piperidin-4-yl)phenyl)amino)-5-((difluoro-13-methyl)-12-fluoraneyl)pyrimidin-4-yl)-3,4-dihydrothieno[2,3-f][1,4]thiazepin-5(2H)-one 1,1-dioxide and acetaldehyde. The title compound was isolated in 58% yield. m/z (ESI, +ve)=646.1 [M+H]⁺. ¹H NMR (400 MHz, CDCl₃) δ 8.74 (s, 1H), 8.16-8.02 (m, 2H), 7.88 (s, 1H), 7.21 (d, J=8.4 Hz, 1H), 7.08 (s, 1H), 3.97 (t, J=6.0 Hz, 2H), 3.72-3.56 (m, 4H), 3.17-3.02 (m, 2H), 2.98-2.91 (m, 1H), 2.81-2.71 (m, 2H), 2.42-2.20 (m, 3H), 2.11-2.05 (m, 2H), 1.89-1.84 (m, 1H), 1.46 (t, J=7.2 Hz, 3H), 1.26 (s, 2H), 1.12-0.96 (m, 4H), 0.73 (q, J=5.2 Hz, 2H).

### Example 254: 4-cyclopropyl-7-(2-((2-cyclopropyl-4-(1-(oxetan-3-ylmethyl)piperidin-4-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((2-cyclopropyl-4-(piperidin-4-yl)phenyl)amino)-5-(trifluoromethyl)pyrimidin-4-yl)-3,4-dihydrothieno[2,3-f][1,4]thiazepin-5(2H)-one 1,1-dioxide and oxetane-3-carbaldehyde. The title compound was isolated in 79% yield. m/z (ESI, +ve)=688.4 [M+H]⁺. ¹H NMR (400 MHz, CDCl₃) δ 8.73 (s, 1H), 8.07 (s, 2H), 7.86 (s, 1H), 7.18 (d, J=7.6 Hz, 1H), 7.06 (s, 1H), 5.03-4.69 (m, 4H), 4.52-4.42 (m, 3H), 3.99-3.90 (m, 3H), 3.65 (t, J=6.0 Hz, 2H), 3.41-3.27 (m, 1H), 3.26-2.90 (m, 4H), 2.82 (d, J=5.6 Hz, 2H), 2.49 (dd, J=6.4, 12.0 Hz, 1H), 2.16-2.07 (m, 2H), 1.26 (s, 2H), 1.06-0.96 (m, 4H), 0.76-0.68 (m, 2H).

### Example 255: 4-cyclopropyl-7-(2-((2-cyclopropyl-4-(4-ethylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((2-cyclopropyl-4-(piperazin-1-yl)phenyl)amino)-5-(trifluoromethyl)pyrimidin-4-yl)-3,4-dihydrothieno[2,3-f][1,4]thiazepin-5(2H)-one 1,1-dioxide and acetaldehyde. The title compound was isolated in 73% yield. m/z (ESI, +ve)=647.2 [M+H]⁺. ¹H NMR (400 MHz, CDCl₃) δ 8.70 (s, 1H), 8.05 (s, 1H), 7.92-7.88 (m, 1H), 7.70-7.68 (m, 1H), 6.91-6.89 (m, 1H), 6.76 (d, J=2.4 Hz, 1H), 3.96-3.93 (m, 2H), 3.66 (t, J=6.0 Hz, 2H), 3.29-3.27 (m, 4H), 2.93-2.91 (m, 1H), 2.74-2.72 (m, 4H), 2.62-2.58 (m, 2H), 1.71-1.70 (m, 1H), 1.21 (t, J=7.2 Hz, 3H), 1.04-0.99 (m, 2H), 0.95-0.94 (m, 4H), 0.72-0.70 (m, 2H).

### Example 256: 7-(2-((7-cyclopropyl-2-(cyclopropylmethyl)-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-cyclopropyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide and cyclopropanecarbaldehyde. The title compound was isolated in 20% yield. m/z (ESI, +ve)=660.2 [M+H]⁺. ¹H NMR (400 MHz, DMSO-d₆) δ 9.90 (s, 1H), 8.82 (s, 1H), 7.82 (s, 1H), 7.16 (s, 1H), 6.71 (s, 1H), 5.24-5.20 (m, 1H), 4.75-4.67 (m, 4H), 4.41-4.00 (m, 4H), 3.58 (s, 1H), 2.81-2.78 (m, 3H), 2.3 (s, 1H), 1.99-1.91 (m, 2H), 1.04-1.03 (m, 2H), 0.87-0.80 (m, 3H), 0.58-0.57 (m, 4H), 0.17 (s, 2H).

### Example 257: 7-(2-((7-cyclopropyl-2-(oxetan-3-ylmethyl)-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((2-cyclopropyl-4-(piperidin-4-yl)phenyl)amino)-5-(trifluoromethyl)pyrimidin-4-yl)-3,4-dihydrothieno[2,3-f][1,4]thiazepin-5(2H)-one 1,1-dioxide and oxetane-3-carbaldehyde. The title compound was isolated in 28% yield. m/z (ESI, +ve)=676.2 [M+H]⁺. ¹H NMR (400 MHz, DMSO-d₆) δ 9.87 (s, 1H), 8.81 (brs, 1H), 7.82 (s, 1H), 7.12 (s, 1H), 6.66 (s, 1H), 5.24-5.20 (m, 1H), 4.76-4.65 (m, 4H), 4.30 (t, J=6.0 Hz, 2H), 4.01-3.90 (m, 4H), 3.45 (s, 2H), 3.33-3.30 (m, 1H), 2.78-2.73 (m, 4H), 2.61-2.56 (m, 2H), 1.95-1.90 (m, 1H), 0.82-0.79 (m, 2H), 0.56-0.50 (m, 2H).

### Example 258: 7-(2-((6-ethyl-1,2,3,4-tetrahydroisoquinolin-7-yl)amino)-5-(trifluoromethyl)pyrimidin-4-yl)-4-(2,2,2-trifluoroethyl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(6-ethyl-7-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-(2,2,2-trifluoroethyl)-3,4-dihydrothieno[2,3-f][1,4]thiazepin-5(2H)-one 1,1-dioxide in step 3. The title compound was isolated. MS (ESI) m/z: 620.1 [M+H]⁺. ¹H NMR (400 MHz, methanol-d₄) δ 8.70 (s, 1H), 8.00 (s, 1H), 7.26 (s, 1H), 7.12 (s, 1H), 4.36 (q, J=18.4, 9.2 Hz, 2H), 4.13 (s, 2H), 4.09 (t, J=5.6 Hz, 2H), 3.84 (t, J=5.6 Hz, 2H), 3.27 (t, J=6.0 Hz, 2H), 2.96 (t, J=6.0 Hz, 2H), 2.65 (q, J=14.8, 7.2 Hz, 2H), 1.17 (t, J=7.6 Hz, 3H).

### Example 259: 7-(2-((7-chloro-1,2,3,4-tetrahydroisoquinolin-6-yl)amino)-5-methylpyrimidin-4-yl)-4-methyl-3,4-dihydrothieno[2,3-f][1,4]thiazepin-5(2H)-one 1,1-dioxide

### Step 1: 2-chloro-5-methyl-4-(trimethylstannyl)pyrimidine

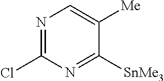

To a solution of 2,4-dichloro-5-methyl-pyrimidine (13 mmol) in dioxane (10 mL), hexamethylditin (25 mmol) and tetrakis(triphenylphosphine)palladium (0.61 mmol) were added in one portion. The reaction mixture was stirred overnight at 90° C., cooled down to room temperature and filtered through a pad of celite. The filtrate was concentrated and purified by silica gel chromatography (0-50% ethyl acetate in hexanes) to afford the title compound in 60% yield. m/z (ESI, +ve)=292.9 [M+H]⁺. ¹H NMR (300 MHz, CDCl₃) δ 0.42 (s, 9H), 2.32 (s, 3H), 8.21 (s, 1H).

### Step 2: 7-(2-chloro-5-methylpyrimidin-4-yl)-4-methyl-3,4-dihydrothieno[2,3-f][1,4]thiazepin-5(2H)-one 1,1-dioxide

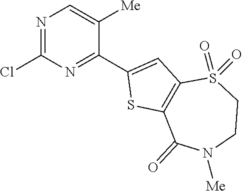 [SMILES: Cc1cnc(Cl)nc1-c1cc2c(s1)C(=O)N(C)CCS2(=O)=O]

The title compound was prepared analogously to Example 155, step 3, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 2-chloro-5-methyl-4-(trimethylstannyl)pyrimidine and 7-bromo-4-methyl-3,4-dihydrothieno[2,3-f][1,4]thiazepin-5(2H)-one 1,1-dioxide in step 3. The title compound was isolated in 42% yield. MS (ESI) m/z: 358.0 [M+H]⁺.

### Step 3: 7-(2-((6-ethyl-1,2,3,4-tetrahydroisoquinolin-7-yl)amino)-5-(trifluoromethyl)pyrimidin-4-yl)-4-(2,2,2-trifluoroethyl)-3,4-dihydrothieno[2,3-f][1,4]thiazepin-5(2H)-one 1,1-dioxide

To a solution of 7-(2-chloro-5-methylpyrimidin-4-yl)-4-methyl-2H,3H,4H,5H-1λ⁶-thieno[2,3-f][1,4]thiazepine-1,1,5-trione (0.27 mmol) in dioxane (2.0 mL), 1-(6-amino-7-chloro-1,2,3,4-tetrahydroisoquinolin-2-yl)-2,2,2-trifluoroethan-1-one (0.32 mmol), 4,5-Bis(diphenylphosphino)-9,9-dimethylxanthene (0.054 mmol), cesium carbonate (0.54 mmol) and bis(dibenzylideneacetone)palladium(0) (0.027 mmol) were added. The reaction mixture was stirred overnight at 100° C., cooled down to room temperature and filtered. The filtrate was concentrated and the crude was purified by silica gel chromatography (0-35% ethyl acetate in dichloromethane) to afford the title compound in 18% yield. MS (ESI) m/z: 504.1 [M+H]⁺. ¹H NMR (300 MHz, DMSO-d₆) δ 8.80 (s, 1H), 8.47 (s, 1H), 7.83 (s, 1H), 7.53 (s, 1H), 7.17 (s, 1H), 3.98-3.87 (m, 4H), 3.82 (s, 2H), 3.57 (s, 2H), 3.09 (s, 3H), 2.94 (t, J=5.9 Hz, 2H), 2.69 (d, J=5.5 Hz, 2H), 2.44 (s, 3H).

### Example 260: 4-cyclopropyl-7-(2-((7-cyclopropyl-1,2,3,4-tetrahydroisoquinolin-6-yl)amino)-5-methylpyrimidin-4-yl)-3,4-dihydrothieno[2,3-f][1,4]thiazepin-5(2H)-one 1,1-dioxide

The title compound was prepared analogously to Example 259, where 7-bromo-4-cyclopropyl-3,4-dihydrothieno[2,3-f][1,4]thiazepin-5(2H)-one 1,1-dioxide was replaced with 7-bromo-4-cyclopropyl-3,4-dihydrothieno[2,3-f][1,4]thiazepin-5(2H)-one 1,1-dioxide in step 2 and 1-(6-amino-7-chloro-1,2,3,4-tetrahydroisoquinolin-2-yl)-2,2,2-trifluoroethan-1-one with 7-cyclopropyl-1,2,3,4-tetrahydroisoquinolin-6-amine in step 3. The title compound was isolated. MS (ESI) m/z: 536.0 [M+H]⁺. ¹H NMR (300 MHz, DMSO-d₆) δ 0.54 (m, 2H), 0.80 (m, 6H), 1.94 (m, 1H), 2.42 (s, 3H), 2.67 (m, 2H), 2.91 (m, 3H), 3.78 (s, 2H), 3.86 (s, 4H), 6.65 (s, 1H), 7.42 (s, 1H), 7.80 (s, 1H), 8.43 (s, 1H), 8.62 (s, 1H).

### Example 261: 4-cyclopropyl-7-(5-cyclopropyl-2-((7-cyclopropyl-1,2,3,4-tetrahydroisoquinolin-6-yl)amino)pyrimidin-4-yl)-3,4-dihydrothieno[2,3-f][1,4]thiazepin-5(2H)-one 1,1-dioxide

The title compound was prepared analogously to Example 260, where 2,4-dichloro-5-methyl-pyrimidine was replaced with 2,4-dichloro-5-cyclopropylpyrimidine in step 1. The title compound was isolated. MS (ESI) m/z: 562.4 [M+H]⁺. ¹H NMR (300 MHz, DMSO-d₆) δ 0.54 (m, 2H), 0.78 (m, 8H), 1.01 (m, 2H), 1.99 (m, 2H), 2.66 (t, J=5.9 Hz, 2H), 2.91 (m, 3H), 3.78 (s, 2H), 3.87 (s, 4H), 6.64 (s, 1H), 7.40 (s, 1H), 8.21 (s, 1H), 8.40 (s, 1H), 8.68 (s, 1H).

### Example 262: 7-(2-((6-ethyl-1,2,3,4-tetrahydroisoquinolin-7-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(6-ethyl-7-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-(oxetan-3-yl)-3,4-dihydrothieno[2,3-f][1,4]thiazepin-5(2H)-one 1,1-dioxide in step 3. The title compound was isolated. MS (ESI) m/z: 594.2 [M+H]⁺. ¹H NMR (400 MHz, DMSO-d₆) δ 9.86 (s, 1H), 8.79 (s, 1H), 7.83 (s, 1H), 7.08-7.03 (m, 2H), 5.26-5.19 (m, 1H), 4.76-4.68 (m, 4H), 4.02 (s, 4H), 3.91 (s, 2H), 3.06 (t, J=4.8 Hz, 2H), 2.76 (d, J=4.8 Hz, 2H), 2.54-2.52 (m, 3H), 1.08 (t, J=7.6 Hz, 3H).

### Example 263: 4-cyclopropyl-7-(2-((7-isopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

### Step 1: 2,2,2-trifluoro-1-(6-nitro-7-(prop-1-en-2-yl)-3,4-dihydroisoquinolin-2(1H)-yl)ethan-1-one

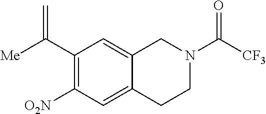 [SMILES: C=C(C)c1cc2c(cc1[N+](=O)[O-])CCN(C(=O)C(F)(F)F)C2]

The title compound was prepared analogously to Example 212, step 3 where 1-(6-bromo-7-nitro-3,4-dihydro-1H-isoquinolin-2-yl)-2,2,2-trifluoro-ethanone and tributyl(vinyl)stannane were replaced with 1-(7-bromo-6-nitro-3,4-dihydroisoquinolin-2(1H)-yl)-2,2,2-trifluoroethan-1-one and tributyl(prop-1-en-2-yl)stannane. The title compound was isolated in 49% yield. MS (ESI) m/z: 315.1 [M+H]⁺.

### Step 2: 1-(6-amino-7-(prop-1-en-2-yl)-3,4-dihydroisoquinolin-2(1H)-yl)-2,2,2-trifluoroethan-1-one

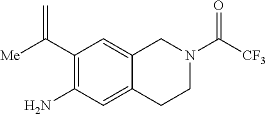 [SMILES: C=C(C)c1cc2c(cc1N)CCN(C(=O)C(F)(F)F)C2]

To a solution of 2,2,2-trifluoro-1-(6-nitro-7-(prop-1-en-2-yl)-3,4-dihydroisoquinolin-2(1H)-yl)ethan-1-one (3.1 mmol) in methanol (30 mL) was added 10% palladium on carbon (300 mg). The mixture was hydrogenated at 50 psi for 2 hours, filtered through celite and concentrated to afford a residue that was purified by preparative. The title compound was isolated in 45% yield as a purple oil. MS (ESI) m/z: 285.5[M+H]⁺.

### Step 3: 1-(6-amino-7-isopropyl-3,4-dihydroisoquinolin-2(1H)-yl)-2,2,2-trifluoroethan-1-one

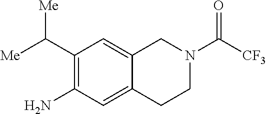 [SMILES: CC(C)c1cc2c(cc1N)CCN(C(=O)C(F)(F)F)C2]

To a solution of 1-(6-amino-7-isopropenyl-3,4-dihydro-1H-isoquinolin-2-yl)-2,2,2-trifluoro-ethanone (1.1 mmol) in methanol (20 mL) was hydrogenated at 50 psi in the presence of 10% palladium on carbon (100 mg) for 12 hours. The mixture was filtered through celite and concentrated to afford the title compound in 83% yield. MS (ESI) m/z: 287.1[M+H]⁺.

### Step 4: 1-(6-((4-chloro-5-(trifluoromethyl)pyrimidin-2-yl)amino)-7-isopropyl-3,4-dihydroisoquinolin-2(1H)-yl)-2,2,2-trifluoroethan-1-one

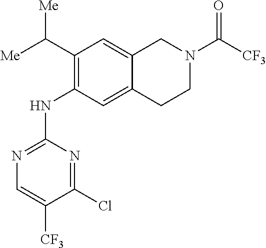 [SMILES: CC(C)c1cc2c(cc1Nc1ncc(C(F)(F)F)c(Cl)n1)CCN(C(=O)C(F)(F)F)C2]

The title compound was prepared analogously to Example 1, step 5 where 1-(6-amino-7-chloro-1,2,3,4-tetrahydroisoquinolin-2-yl)-2,2,2-trifluoroethan-1-one was replaced with 1-(6-amino-7-isopropyl-3,4-dihydroisoquinolin-2(1H)-yl)-2,2,2-trifluoroethan-1-one. The title compound was isolated in 25% yield. MS (ESI) m/z: 467.3[M+H]⁺.

### Step 5: 2,2,2-trifluoro-1-(7-isopropyl-6-((5-(trifluoromethyl)-4-(trimethylstannyl)pyrimidin-2-yl)amino)-3,4-dihydroisoquinolin-2(1H)-yl)ethan-1-one

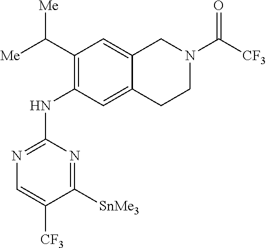

The title compound was prepared analogously to Example 4, step 1 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with 1-(6-((4-chloro-5-(trifluoromethyl)pyrimidin-2-yl)amino)-7-isopropyl-3,4-dihydroisoquinolin-2(1H)-yl)-2,2,2-trifluoroethan-1-one. The title compound was isolated in 31% yield as a colorless oil. MS (ESI) m/z: 597.3 [M+H]⁺.

### Step 6-7: 4-cyclopropyl-7-(2-((7-isopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one was replaced with 2,2,2-trifluoro-1-(7-isopropyl-6-((5-(trifluoromethyl)-4-(trimethylstannyl)pyrimidin-2-yl)amino)-3,4-dihydroisoquinolin-2(1H)-yl)ethan-1-one in step 3. The title compound was isolated. MS (ESI) m/z: 592.2[M+H]⁺. ¹H NMR (400 MHz, CDCl₃) δ 8.71 (s, 1H), 8.06 (s, 1H), 7.67 (d, J=2.8 Hz, 1H), 7.26-7.16 (m, 1H), 7.09 (s, 1H), 4.39 (s, 2H), 3.95 (t, J=5.6 Hz, 2H), 3.64 (t, J=5.6 Hz, 2H), 3.52 (t, J=5.6 Hz, 2H), 3.21 (t, J=5.6 Hz, 2H), 3.17-3.08 (m, 1H), 2.95-2.88 (m, 1H), 1.26 (s, 6H), 0.98-0.91 (m, 4H).

### Example 264: 4-cyclopropyl-7-(2-((2-ethyl-4-(piperazin-1-yl)phenyl)amino)-5-(trifluoromethyl)pyrimidin-4-yl)-3,4-dihydrothieno[2,3-f][1,4]thiazepin-5(2H)-one 1,1-dioxide

### Step 1: tert-butyl 4-(4-nitro-3-vinylphenyl)piperazine-1-carboxylate

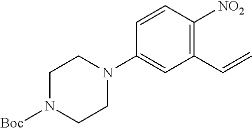 [SMILES: C=Cc1cc(N2CCN(C(=O)OC(C)(C)C)CC2)ccc1[N+](=O)[O-]]

The title compound was prepared analogously to Example 155, step 3, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one, and 7-bromo-4-cyclopropyl-3,4-dihydrothieno[2,3-f][1,4]thiazepin-5(2H)-one 1,1-dioxide were replaced with tributyl(vinyl)stannane and tert-butyl 4-(3-bromo-4-nitrophenyl)piperazine-1-carboxylate in step 3. The title compound was isolated. ¹H NMR (400 MHz, CDCl₃) δ ppm 1.49 (s, 9H) 3.39-3.45 (m, 4H) 3.58-3.64 (m, 4H) 5.42 (dd, J=10.8, 1.2 Hz, 1H) 5.61 (dd, J=17.2, 1.2 Hz, 1H) 6.72-6.87 (m, 2H) 7.37 (dd, J=17.2, 10.8 Hz, 1H) 8.06 (d, J=9.2 Hz, 1H).

### Step 2: tert-butyl 4-(4-amino-3-ethylphenyl)piperazine-1-carboxylate

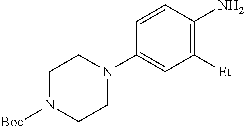 [SMILES: CCc1cc(N2CCN(C(=O)OC(C)(C)C)CC2)ccc1N]

The title compound was prepared analogously to Example 263, step 5 where 1-(6-amino-7-isopropenyl-3,4-dihydro-1H-isoquinolin-2-yl)-2,2,2-trifluoro-ethanone was replaced with tert-butyl 4-(4-nitro-3-vinylphenyl)piperazine-1-carboxylate. The title compound was isolated in 66% yield. MS (ESI) m/z: 306.4 [M+H]⁺. ¹H NMR (400 MHz, CDCl₃) δ ppm 1.25 (t, J=7.6 Hz, 3H) 1.49 (s, 9H) 2.51 (q, J=7.6 Hz, 2H) 2.99 (br s, 4H) 3.55-3.67 (m, 4H) 6.56-6.73 (m, 2H) 6.76 (br s, 1H).

### Step 3: tert-butyl 4-(4-((4-chloro-5-(trifluoromethyl)pyrimidin-2-yl)amino)-3-ethylphenyl)piperazine-1-carboxylate

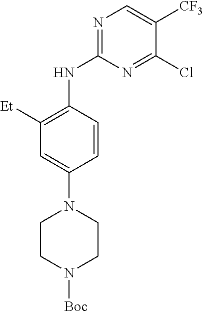 [SMILES: CCc1cc(N2CCN(C(=O)OC(C)(C)C)CC2)ccc1Nc1ncc(C(F)(F)F)c(Cl)n1]

The title compound was prepared analogously to Example 1, step 5 where 1-(6-amino-7-chloro-1,2,3,4-tetrahydroisoquinolin-2-yl)-2,2,2-trifluoroethan-1-one was replaced with tert-butyl 4-(4-amino-3-ethylphenyl)piperazine-1-carboxylate. The title compound was isolated in 42% yield. MS (ESI) m/z: 486.4 [M+H]⁺. ¹H NMR (400 MHz, CDCl₃) δ ppm 1.22 (t, J=7.6 Hz, 3H) 1.50 (s, 9H) 2.60 (q, J=7.6 Hz, 2H) 3.17 (d, J=4.4 Hz, 4H) 3.59 (d, J=4.4 Hz, 4H) 6.77-6.91 (m, 2H) 7.04 (br s, 1H) 7.32-7.55 (m, 1H) 8.48 (s, 1H).

### Step 4: tert-butyl 4-(3-ethyl-4-((5-(trifluoromethyl)-4-(trimethylstannyl)pyrimidin-2-yl)amino)phenyl)piperazine-1-carboxylate

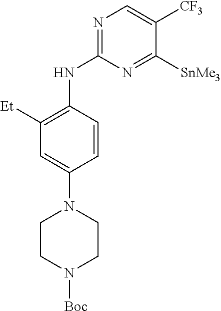

The title compound was prepared analogously to Example 4, step 1 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with tert-butyl 4-(4-((4-chloro-5-(trifluoromethyl)pyrimidin-2-yl)amino)-3-ethylphenyl)piperazine-1-carboxylate. The title compound was isolated in 44% yield. MS (ESI) m/z: 614.4 [M+H]⁺.

### Step 5: 4-cyclopropyl-7-(2-((2-ethyl-4-(piperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 149, steps 5-6, where tert-butyl 4-(4-chloro-3-((5-(trifluoromethyl)-4-(trimethylstannyl)pyrimidin-2-yl)amino)phenyl)piperidine-1-carboxylate and 7-bromo-4-methyl-3,4-dihydrothieno[2,3-f][1,4]thiazepin-5(2H)-one 1,1-dioxide were replaced with tert-butyl 4-(3-ethyl-4-((5-(trifluoromethyl)-4-(trimethylstannyl)pyrimidin-2-yl)amino)phenyl)piperazine-1-carboxylate and 7-bromo-4-cyclopropyl-3,4-dihydrothieno[2,3-f][1,4]thiazepin-5(2H)-one 1,1-dioxide in step 5. The title compound was isolated. MS (ESI) m/z: 607.3 [M+H]⁺. ¹H NMR (400 MHz, CDCl₃) δ ppm 0.90-0.97 (m, 4H) 1.23 (t, J=7.6 Hz, 4H) 2.62-2.67 (m, 2H) 2.87-2.94 (m, 1H) 3.15-3.51 (m, 8H) 3.64 (t, J=5.6 Hz, 2H) 3.93 (br s, 2H) 6.79-6.93 (m, 2H) 7.15 (br s, 1H) 7.40-7.67 (m, 1H) 8.03 (s, 1H) 8.66 (s, 1H).

### Example 265: 4-cyclopropyl-7-(2-((4-(4-(cyclopropylmethyl)piperazin-1-yl)-2-ethylphenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((2-ethyl-4-(piperazin-1-yl)phenyl)amino)-5-(trifluoromethyl)pyrimidin-4-yl)-3,4-dihydrothieno[2,3-f][1,4]thiazepin-5(2H)-one 1,1-dioxide and cyclopropanecarbaldehyde. The title compound was isolated in 53% yield. m/z (ESI, +ve)=661.3 [M+H]⁺. ¹H NMR (400 MHz, CDCl₃) δ ppm 0.26 (d, J=4.0 Hz, 2H) 0.65 (d, J=7.2 Hz, 2H) 0.89-1.09 (m, 5H) 1.23 (t, J=7.6 Hz, 3H) 2.56-2.70 (m, 4H) 2.83-3.07 (m, 5H) 3.38 (br s, 4H) 3.64 (t, J=5.6 Hz, 2H) 3.93 (br s, 2H) 6.77-6.95 (m, 2H) 7.10 (br s, 1H) 7.39-7.70 (m, 1H) 8.02 (s, 1H) 8.66 (s, 1H).

### Example 266: 4-cyclopropyl-7-(2-((2-ethyl-4-(4-(oxetan-3-ylmethyl)piperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((2-ethyl-4-(piperazin-1-yl)phenyl)amino)-5-(trifluoromethyl)pyrimidin-4-yl)-3,4-dihydrothieno[2,3-f][1,4]thiazepin-5(2H)-one 1,1-dioxide and oxetane-3-carbaldehyde. The title compound was isolated in 46% yield. m/z (ESI, +ve)=677.2 [M+H]⁺. ¹H NMR (400 MHz, CDCl₃) δ ppm 0.87-1.02 (m, 4H) 1.22 (t, J=7.6 Hz, 3H) 2.53-2.71 (m, 6H) 2.78-2.95 (m, 3H) 3.24 (br s, 4H) 3.30-3.40 (m, 1H) 3.64 (t, J=5.2 Hz, 2H) 3.91 (d, J=6.8 Hz, 2H) 4.45-4.49 (m, 2H) 4.85 (dd, J=7.6, 6.0 Hz, 2H) 6.83 (br s, 2H) 7.11 (br s, 1H) 7.32-7.65 (m, 1H) 8.02 (s, 1H) 8.65 (s, 1H).

### Example 267: 4-cyclopropyl-7-(2-((2-ethyl-7-isopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((7-isopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and acetaldehyde. The title compound was isolated in 85% yield. m/z (ESI, +ve)=620.2 [M+H]⁺. ¹H NMR (400 MHz, CDCl₃) δ 8.70 (s, 1H), 8.05 (s, 1H), 7.62-7.39 (m, 1H), 7.23-7.19 (m, 1H), 7.04 (s, 1H), 4.05-3.89 (m, 4H), 3.64 (t, J=5.8 Hz, 2H), 3.20-3.04 (m, 5H), 2.99-2.89 (m, 3H), 1.41-1.36 (m, 3H), 1.25 (d, J=6.8 Hz, 6H), 0.98-0.92 (m, 4H).

### Example 268: 4-cyclopropyl-7-(2-((2-(cyclopropylmethyl)-7-isopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((7-isopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and cyclopropanecarbaldehyde. The title compound was isolated in 84% yield. m/z (ESI, +ve)=646.4 [M+H]⁺. ¹H NMR (400 MHz, CDCl₃) δ 8.68 (s, 1H), 8.04 (s, 1H), 7.58-7.40 (m, 1H), 7.37-7.28 (m, 1H), 7.23-7.17 (m, 1H), 7.04 (s, 1H), 4.00-3.84 (m, 4H), 3.64 (t, J=5.6 Hz, 2H), 3.11 (d, J=6.8 Hz, 1H), 3.09-2.95 (m, 4H), 2.93-2.88 (m, 1H), 2.69-2.57 (m, 2H), 1.25 (d, J=6.8 Hz, 6H), 1.10-1.05 (m, 1H), 0.98-0.92 (m, 4H), 0.66 (d, J=7.6 Hz, 2H), 0.28 (d, J=4.8 Hz, 2H).

### Example 269: 4-cyclopropyl-7-(2-((7-isopropyl-2-(oxetan-3-ylmethyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((7-isopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and cycloprooxetane-3-carbaldehyde. The title compound was isolated in 79% yield. MS (ESI) m/z: 662.2 [M+H]⁺. ¹H NMR (400 MHz, CDCl₃) δ 8.59 (s, 1H), 7.97 (s, 1H), 7.43-7.21 (m, 1H), 7.10-7.03 (m, 1H), 6.91 (s, 1H), 4.81-4.72 (m, 3H), 4.41 (t, J=6.0 Hz, 2H), 3.87-3.83 (m, 2H), 3.62-3.46 (m, 4H), 3.34-3.22 (m, 1H), 3.07-2.96 (m, 1H), 2.86-2.81 (m, 4H), 2.71-2.59 (m, 2H), 1.16 (d, J=6.8 Hz, 6H), 0.92-0.85 (m, 4H).

### Example 270: 7-(2-((6-cyclopropyl-1,2,3,4-tetrahydroisoquinolin-7-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(6-cyclopropyl-7-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-(oxetan-3-yl)-3,4-dihydrothieno[2,3-f][1,4]thiazepin-5(2H)-one 1,1-dioxide in step 3. The title compound was isolated. MS (ESI) m/z: 606.2 [M+H]⁺. ¹H NMR (400 MHz, methanol-d₄) δ 8.76 (s, 1H), 8.02 (s, 1H), 7.56 (s, 1H), 6.96 (s, 1H), 5.29 (t, J=7.2 Hz, 1H), 4.91 (s, 2H), 4.84 (s, 2H), 4.30 (s, 2H), 4.07-4.04 (m, 2H), 3.91-3.89 (m, 2H), 3.43 (t, J=6.4 Hz, 2H), 3.04 (t, J=6.0 Hz, 2H), 2.00-1.93 (m, 1H), 0.99-0.94 (m, 2H), 0.67-0.63 (m, 2H).

### Example 271: 4-cyclopropyl-7-(2-((2-ethyl-4-(4-ethylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((2-ethyl-4-(piperazin-1-yl)phenyl)amino)-5-(trifluoromethyl)pyrimidin-4-yl)-3,4-dihydrothieno[2,3-f][1,4]thiazepin-5(2H)-one 1,1-dioxide and acetaldehyde. The title compound was isolated in 58% yield. m/z (ESI, +ve)=635.4 [M+H]⁺. ¹H NMR (400 MHz, CDCl₃) δ ppm 0.87-1.00 (m, 4H) 1.19-1.30 (m, 6H) 2.64 (q, J=7.6 Hz, 2H) 2.69-2.79 (m, 2H) 2.79-3.00 (m, 5H) 3.37 (br s, 4H) 3.64 (t, J=5.6 Hz, 2H) 3.93 (br s, 2H) 6.77-6.92 (m, 2H) 7.08 (br s, 1H) 7.37-7.70 (m, 1H) 8.03 (s, 1H) 8.66 (s, 1H).

### Example 272: 4-cyclopropyl-7-(2-((2-ethyl-4-(4-isopropylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

To a solution of 4-cyclopropyl-7-(2-((2-ethyl-4-(piperazin-1-yl)phenyl)amino)-5-(trifluoromethyl)pyrimidin-4-yl)-3,4-dihydrothieno[2,3-f][1,4]thiazepin-5(2H)-one 1,1-dioxide (0.03 mmol) in methyl alcohol (1 mL) and acetic acid (0.1 mL) was added acetone (0.10 mmol). The mixture was stirred at room temperature for 30 minutes and sodium cyanoborohydride (0.07 mmol) was added. After 1 hour, the reaction was filtered and concentrated to afford a residue that was purified by preparative TLC. The title compound was isolated in 59% yield. MS (ESI) m/z: 649.2 [M+H]⁺. ¹H NMR (400 MHz, DMSO-d₆) δ ppm 0.79 (br s, 4H) 1.01 (d, J=6.4 Hz, 6H) 1.08 (t, J=7.6 Hz, 3H) 2.52-2.62 (m, 6H) 2.65-2.73 (m, 1H) 2.87 (br dd, J=5.6, 3.6 Hz, 1H) 3.13 (d, J=4.0 Hz, 4H) 3.78-3.97 (m, 4H) 6.66-6.89 (m, 2H) 6.98-7.30 (m, 1H) 7.64-7.91 (m, 1H) 8.53-8.96 (m, 1H) 9.52-9.82 (m, 1H).

### Example 273: 4-cyclopropyl-7-(2-((7-(trifluoromethoxy)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

### Step 1-5: 1-(6-((4-chloro-5-(trifluoromethyl)pyrimidin-2-yl)amino)-7-(trifluoromethoxy)-3,4-dihydroisoquinolin-2(1H)-yl)-2,2,2-trifluoroethan-1-one

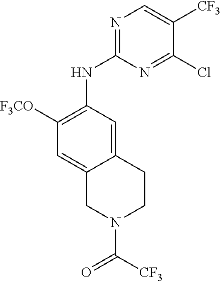 [SMILES: O=C(N1CCc2cc(Nc3ncc(C(F)(F)F)c(Cl)n3)c(OC(F)(F)F)cc2C1)C(F)(F)F]

The title compound was prepared analogously to Example 1, steps 1-5, where 2-(4-chlorophenyl)ethylamine was replaced with 2-[4-(trifluoromethoxy)phenyl]ethanamine in step 1. The title compound was isolated. MS (ESI) m/z: 509.0 [M+H]⁺.

### Step 6: 7-(trifluoromethoxy)-N-(5-(trifluoromethyl)-4-(trimethylstannyl)pyrimidin-2-yl)-1,2,3,4-tetrahydroisoquinolin-6-amine

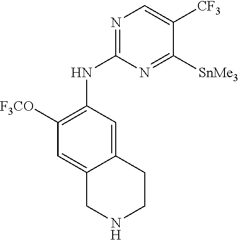

The title compound was prepared analogously to Example 4, step 1 where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with 1-(6-((4-chloro-5-(trifluoromethyl)pyrimidin-2-yl)amino)-7-(trifluoromethoxy)-3,4-dihydroisoquinolin-2(1H)-yl)-2,2,2-trifluoroethan-1-one. The title compound was isolated in 27% yield. MS (ESI) m/z: 639.0 [M+H]⁺.

### Step 7: 4-cyclopropyl-7-(2-((7-(trifluoromethoxy)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one was replaced with 7-(trifluoromethoxy)-N-(5-(trifluoromethyl)-4-(trimethylstannyl)pyrimidin-2-yl)-1,2,3,4-tetrahydroisoquinolin-6-amine in step 3. The title compound was isolated. MS (ESI) m/z: 634.1 [M+H]⁺. ¹H NMR (400 MHz, CDCl₃) δ 8.79 (s, 1H), 8.23 (s, 1H), 8.10 (s, 1H), 7.63 (s, 1H), 7.02 (s, 1H), 4.11 (s, 2H), 3.97 (t, J=5.6 Hz, 2H), 3.66 (t, J=6.0 Hz, 2H), 3.29 (t, J=5.6 Hz, 2H), 3.07-2.99 (m, 2H), 2.96-2.92 (m, 1H), 1.01-0.91 (m, 4H).

### Example 274: 7-(2-((2-(cyclopropylmethyl)-7-ethyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-ethyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide and cyclopropanecarbaldehyde. The title compound was isolated in 56% yield. m/z (ESI, +ve)=648.2 [M+H]⁺. ¹H NMR (400 MHz, methanol-d₄) δ 8.71 (s, 1H), 8.01 (s, 1H), 7.36-7.26 (m, 1H), 7.08 (s, 1H), 5.32-5.25 (m, 1H), 4.04 (t, J=5.6 Hz, 2H), 3.96 (s, 2H), 3.90-3.86 (m, 2H), 3.08-3.03 (m, 4H), 2.68-2.60 (m, 4H), 1.29 (s, 3H), 1.17 (t, J=7.6 Hz, 3H), 1.11-1.04 (m, 1H), 0.93-0.84 (m, 1H), 0.70-0.64 (m, 2H), 0.34-0.29 (m, 2H).

### Example 275: 7-(2-((7-ethyl-2-(oxetan-3-ylmethyl)-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-ethyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide and oxetane-3-carbaldehyde. The title compound was isolated in 48% yield. m/z (ESI, +ve)=664.2 [M+H]⁺. ¹H NMR (400 MHz, methanol-d₄) δ 8.69 (s, 1H), 8.00 (s, 1H), 7.24 (s, 1H), 7.01 (s, 1H), 5.30 (t, J=7.2 Hz, 1H), 4.81-4.77 (m, 1H), 4.75-4.69 (m, 1H), 4.59-4.53 (m, 1H), 4.52-4.46 (m, 3H), 4.02 (d, J=5.6 Hz, 2H), 3.89-3.84 (m, 2H), 3.76 (d, J=6.4 Hz, 1H), 3.63 (s, 2H), 3.46-3.39 (m, 1H), 3.17-3.06 (m, 1H), 2.96-2.90 (m, 4H), 2.80-2.74 (m, 2H), 2.62 (q, J=7.6 Hz, 2H), 1.16 (t, J=7.6 Hz, 3H).

### Example 276: 2-(7-cyclopropyl-6-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)-3,4-dihydroisoquinolin-2(1H)-yl)acetonitrile

A mixture of 4-cyclopropyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide (0.02 mmol), 2-bromoacetonitrile (0.03 mmol) and triethylamine (0.05 mmol) in N,N-dimethylformamide (0.5 mL) was stirred at room temperature for 12 hours. The mixture was filtered and concentrated, and the residue was purified by preparative TLC (15% methanol in dichloromethane). The title compound was isolated in 47% yield. MS (ESI) m/z: 629.2[M+H]⁺. ¹H NMR (400 MHz, CDCl₃) δ 8.75 (s, 1H), 8.09 (s, 1H), 8.01 (s, 1H), 7.90 (s, 1H), 6.89 (s, 1H), 3.96 (t, J=5.6 Hz, 2H), 3.80 (s, 2H), 3.76 (s, 2H), 3.66 (t, J=6.0 Hz, 2H), 3.12-3.04 (m, 2H), 2.98-2.89 (m, 3H), 1.88-1.82 (m, 1H), 1.09-1.03 (m, 2H), 1.00-0.93 (m, 4H), 0.71-0.65 (m, 2H).

### Example 277: 4-cyclopropyl-7-(2-((7-ethyl-2-(oxetan-3-ylmethyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-ethyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide and oxetane-3-carbaldehyde. The title compound was isolated in 21% yield. m/z (ESI, +ve)=648.2 [M+H]⁺. ¹H NMR (400 MHz, methanol-d₄) δ=8.73-8.65 (m, 1H), 7.97 (s, 1H), 7.25-7.24 (m, 1H), 7.02 (s, 1H), 4.56-4.44 (m, 4H), 3.95 (m, 2H), 3.77-3.72 (m, 3H), 3.66-3.61 (m, 2H), 3.45-3.39 (m, 1H), 2.95-2.92 (m, 4H), 2.80-2.75 (m, 2H), 2.66-2.60 (m, 2H), 1.16 (t, J=7.6 Hz, 3H), 0.92-0.89 (m, 4H).

### Example 278: 2-(7-cyclopropyl-6-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)-3,4-dihydroisoquinolin-2(1H)-yl)acetamide

To a solution of 4-cyclopropyl-7-[2-[(7-cyclopropyl-1,2,3,4-tetrahydroisoquinolin-6-yl)amino]-5-(trifluoromethyl)pyrimidin-4-yl]-1,1-dioxo-2,3-dihydrothieno[2,3-f][1,4]thiazepin-5-one (0.05 mmol) in N,N-dimethylformamide (0.5 mL) was added 1,8-diazabicyclo[5,4,0]undec-7-ene (0.10 mmol) and 2-bromoacetamide (0.13 mmol). The mixture was stirred for 12 hours, filtered and concentrated. The residue was purified by preparative TLC (15% methanol in dichloromethane). The title compound was isolated in 48% yield. MS (ESI) m/z: 646.2 [M+H]⁺. H NMR (400 MHz, CD₃OD) 8.74 (s, 1H), 8.00 (s, 1H), 7.49 (s, 1H), 6.81 (s, 1H), 3.99 (t, J=5.6 Hz, 2H), 3.85-3.73 (m, 4H), 3.25 (s, 2H), 3.06-2.96 (m, 2H), 2.96-2.86 (m, 3H), 2.03-1.87 (m, 1H), 0.94-0.92 (m, 4H), 0.69-0.59 (m, 2H).

### Example 279: 4-cyclopropyl-7-(2-((7-cyclopropyl-2-((3-methyloxetan-3-yl)methyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and 3-methyloxetane-3-carbaldehyde. The title compound was isolated in 63% yield. m/z (ESI, +ve)=674.2 [M+H]⁺. ¹H NMR (400 MHz, CDCl₃) δ 8.74 (s, 1H), 8.09 (s, 1H), 7.96-7.90 (m, 1H), 7.89-7.85 (m, 1H), 6.84 (s, 1H), 4.57 (d, J=5.6 Hz, 2H), 4.41 (d, J=5.6 Hz, 2H), 3.96 (t, J=6.0 Hz, 2H), 3.65 (t, J=6.0 Hz, 2H), 3.51 (s, 2H), 2.97-2.92 (m, 3H), 2.75 (s, 2H), 2.66 (t, J=5.6 Hz, 2H), 1.84-1.83 (m, 1H), 1.45 (s, 3H), 1.04-1.02 (m, 2H), 0.99-0.95 (m, 4H), 0.69-0.68 (m, 2H).

### Example 280: 2-(4-(4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)-3-ethylphenyl)piperazin-1-yl)acetamide

The title compound was prepared analogously to Example 278, where 4-cyclopropyl-7-[2-[(7-cyclopropyl-1,2,3,4-tetrahydroisoquinolin-6-yl)amino]-5-(trifluoromethyl)pyrimidin-4-yl]-1,1-dioxo-2,3-dihydrothieno[2,3-f][1,4]thiazepin-5-one was replaced with 4-cyclopropyl-7-(2-((2-ethyl-4-(piperazin-1-yl)phenyl)amino)-5-(trifluoromethyl)pyrimidin-4-yl)-3,4-dihydrothieno[2,3-f][1,4]thiazepin-5(2H)-one 1,1-dioxide. The title compound was isolated in 36% yield. MS (ESI) m/z: 664.2 [M+H]+¹H NMR (400 MHz, CDCl₃) δ ppm 0.89-1.00 (m, 4H) 1.24 (t, J=7.6 Hz, 3H) 2.65 (q, J=7.6 Hz, 2H) 2.71-2.80 (m, 4H) 2.87-2.97 (m, 1H) 3.11 (s, 2H) 3.21-3.32 (m, 4H) 3.64 (brt, J=6.0 Hz, 2H) 3.93 (br s, 2H) 5.36-5.52 (m, 1H) 6.85 (br d, J=2.4 Hz, 2H) 6.98-7.14 (m, 2H) 7.38-7.70 (m, 1H) 8.04 (s, 1H) 8.67 (s, 1H).

### Example 281: 4-cyclopropyl-7-(2-((4-(1-(2,2-difluoroethyl)piperidin-4-yl)-2-ethylphenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 240, where tert-butyl 4-(4-amino-3-cyclopropylphenyl)piperidine-1-carboxylate was replaced with tert-butyl 4-(4-amino-3-ethylphenyl)piperidine-1-carboxylate. The title compound was isolated. MS (ESI) m/z: 606.3[M+H]⁺. ¹H NMR (400 MHz, DMSO-d₆) δ 9.88 (s, 1H), 8.79 (s, 1H), 7.79 (s, 1H), 7.32 (s, 1H), 7.15 (s, 1H), 7.09 (d, J=8.0 Hz, 1H), 3.87 (s, 4H), 3.39 (s, 1H), 3.01 (t, J=11.2 Hz, 2H), 2.87 (d, J=12.4 Hz, 2H), 2.61 (q, J=7.6 Hz, 2H), 2.51 (s, 1H), 1.96 (d, J=12.4 Hz, 2H), 1.87-1.77 (m, 2H), 1.11 (t, J=7.6 Hz, 3H), 0.79 (s, 4H).

### Step 2: 4-cyclopropyl-7-(2-((4-(1-(2,2-difluoroethyl)piperidin-4-yl)-2-ethylphenyl)amino)-5-(trifluoromethyl)pyrimidin-4-yl)-3,4-dihydrothieno[2,3-f][1,4]thiazepin-5(2H)-one 1,1-dioxide

To a solution of 4-cyclopropyl-7-[2-[2-ethyl-4-(4-piperidyl)anilino]-5-(trifluoromethyl)pyrimidin-4-yl]-1,1-dioxo-2,3-dihydrothieno[2,3-f][1,4]thiazepin-5-one (0.13 mmol) in dichloromethane (1 mL) was added triethylamine (0.40 mmol) and 2,2-difluoroethyl trifluoromethanesulfonate (0.14 mmol). The mixture was stirred at 50° C. for 2 hours, cooled down to room temperature and concentrated. The residue was purified by preparative TLC (5% methanol in dichloromethane) to afford the title compound in 38% yield. MS (ESI) m/z: 670.3 [M+H]⁺. ¹H NMR (400 MHz, methanol-d₄) δ 8.66 (s, 1H), 7.96 (s, 1H), 7.33 (d, J=6.4 Hz, 1H), 7.20 (s, 1H), 7.14 (d, J=8.0 Hz, 1H), 6.02 (tt, J=55.6, 4.0 Hz, 1H), 3.95 (s, 2H), 3.75 (t, J=5.6 Hz, 2H), 3.10 (d, J=11.2 Hz, 2H), 2.90-2.87 (m, 1H), 2.80 (m, 2H), 2.66 (q, J=7.6 Hz, 2H), 2.58-2.53 (m, 1H), 2.40-2.34 (m, 2H), 1.86-1.80 (m, 4H), 1.18 (t, J=7.6 Hz, 3H), 0.90-0.89 (m, 4H).

### Example 282: 4-cyclopropyl-7-(2-((2-ethyl-4-(1-(2-fluoroethyl)piperidin-4-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 281, where 2,2-difluoroethyl trifluoromethanesulfonate was replaced with 1-bromo-2-fluoro-ethane in step 2. The title compound was isolated in 32% yield. MS (ESI) m/z: 652.2[M+H]⁺. ¹H NMR (400 MHz, methanol-d₄) δ 8.66 (s, 1H), 7.96 (s, 1H), 7.34-7.33 (m, 1H), 7.21 (s, 1H), 7.15 (d, J=8.0 Hz, 1H), 4.69 (t, J=4.8 Hz, 1H), 4.57 (t, J=4.8 Hz, 1H), 4.00-3.00 (m, 2H), 3.75 (t, J=5.2 Hz, 2H), 3.14 (d, J=11.6 Hz, 2H), 2.91-2.88 (m, 1H), 2.82 (t, J=4.8 Hz, 1H), 2.74 (t, J=4.8 Hz, 1H), 2.66 (q, J=7.6 Hz, 2H), 2.62-2.55 (m, 1H), 2.32-2.25 (m, 2H), 1.90-1.83 (m, 4H), 1.18 (t, J=7.6 Hz, 3H), 0.91-0.90 (m, 4H).

### Example 283: 7-(2-((7-chloro-1,2,3,4-tetrahydroisoquinolin-6-yl)amino)-5-cyclopropylpyrimidin-4-yl)-4-methyl-3,4-dihydrothieno[2,3-f][1,4]thiazepin-5(2H)-one 1,1-dioxide

The title compound was prepared analogously to Example 259, where 2,4-dichloro-5-methyl-pyrimidine was replaced with 2,4-dichloro-5-cyclopropyl-pyrimidine in step 1. The title compound was isolated. ¹H NMR (400 MHz, DMSO-d₆) δ 8.93 (S, 1H), 8.44 (s, 1H), 8.23 (s, 1H), 7.58 (S, 1H), 7.28 (s, 1H), 4.02-3.86 (m, 6H), 3.10 (d, J=8.5 Hz, 5H), 2.80 (t, J=6.1 Hz, 2H), 2.11-1.99 (m, 1H), 1.03 (q, J=5.6, 4.9 Hz, 2H), 0.78 (q, J=5.3 Hz, 2H).

### Example 284: 2-(7-cyclopropyl-6-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)-3,4-dihydroisoquinolin-2(1H)-yl)acetamide

To a solution of 7-(2-((7-cyclopropyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide (0.04 mmol) in dimethylformamide (1 mL) was added 2-bromoacetamide (0.06 mmol) and potassium carbonate (0.08 mmol). The mixture was stirred at room temperature for one hour, concentrated and the residue purified by preparative TLC (95% dichloromethane in methanol). The title compound was isolated in 84% yield. MS (ESI) m/z: 663.4 [M+H]⁺. ¹H NMR (400 MHz, CDCl₃) δ 8.76 (s, 1H), 8.13 (s, 1H), 7.99-7.85 (m, 2H), 6.87 (s, 1H), 5.56-5.50 (m, 1H), 5.50-5.44 (m, 1H), 5.04 (t, J=7.4 Hz, 2H), 4.74 (t, J=6.8 Hz, 2H), 4.14 (t, J=6.0 Hz, 2H), 3.80 (t, J=5.8 Hz, 2H), 3.75 (s, 2H), 3.25 (s, 2H), 3.06-3.00 (m, 2H), 2.97-2.88 (m, 2H), 1.06-1.03 (m, 2H), 0.70-0.66 (m, 2H)

### Example 285: 2-(7-cyclopropyl-6-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)-3,4-dihydroisoquinolin-2(1H)-yl)acetonitrile

The title compound was prepared analogously to Example 284, where 7-(2-((7-cyclopropyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide and 2-bromoacetamide were replaced with 7-(2-((7-cyclopropyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide and 2-bromoacetonitrile. The title compound was isolated in 87% yield. MS (ESI) m/z: 645.1 [M+H]⁺. ¹H NMR (400 MHz, CDCl₃) δ 8.76 (s, 1H), 8.12 (s, 1H), 7.96 (s, 1H), 7.87 (s, 1H), 6.88 (s, 1H), 5.60-5.49 (m, 1H), 5.04 (t, J=7.6 Hz, 2H), 4.74 (t, J=6.8 Hz, 2H), 4.14 (t, J=5.6 Hz, 2H), 3.81-3.77 (m, 2H), 3.76 (s, 2H), 3.73 (s, 2H), 3.08-2.99 (m, 2H), 2.93-2.86 (m, 2H), 1.88-1.79 (m, 1H), 1.08-1.01 (m, 2H), 0.72-0.65 (m, 2H).

### Example 286: 7-(2-((7-cyclopropyl-2-((3-methyloxetan-3-yl)methyl)-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-cyclopropyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide and 3-methyloxetane-3-carbaldehyde. The title compound was isolated in 60% yield. m/z (ESI, +ve)=690.2 [M+H]⁺. ¹H NMR (400 MHz, CDCl₃-d) δ, 8.75 (s, 1H), 8.12 (s, 1H), 7.98-7.75 (m, 2H), 6.83 (s, 1H), 5.55 (quin, J=6.8 Hz, 1H), 5.03 (t, J=7.2 Hz, 2H), 4.74 (br t, J=6.8 Hz, 2H), 4.57 (br d, J=5.2 Hz, 2H), 4.40 (d, J=5.6 Hz, 2H), 4.13 (br t, J=5.6 Hz, 2H), 3.79 (br t, J=5.6 Hz, 2H), 3.51 (s, 2H), 2.95 (br s, 2H), 2.75 (s, 2H), 2.71-2.61 (m, 2H), 1.88-1.77 (m, 1H), 1.45 (s, 3H), 1.03 (br d, J=7.6 Hz, 2H), 0.68 (br d, J=4.8 Hz, 2H).

### Example 287: 4-cyclopropyl-7-(2-((7-cyclopropyl-2-(2-hydroxy-2-methylpropyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

A solution of 4-cyclopropyl-7-[2-[(7-cyclopropyl-1,2,3,4-tetrahydroisoquinolin-6-yl)amino]-5-(trifluoromethyl)pyrimidin-4-yl]-1,1-dioxo-2,3-dihydrothieno[2,3-f][1,4]thiazepin-5-one (0.051 mmol), 2,2-dimethyloxirane (0.51 mmol) and triethylamine (0.25 mmol) in ethanol (1 mL) was stirred at 80° C. for 12 hours. The crude was purified directly by preparative TLC (10% methanol in dichloromethane) to afford the title compound in 64% yield. MS (ESI) m/z: 662.3 [M+H]⁺. ¹H NMR (400 MHz, CDCl₃) δ 8.74 (s, 1H), 8.09 (s, 1H), 7.96 (s, 1H), 7.87 (s, 1H), 6.85 (s, 1H), 3.96 (t, J=6.0 Hz, 2H), 3.80 (s, 2H), 3.65 (t, J=6.0 Hz, 2H), 2.99-2.91 (m, 5H), 2.52 (s, 2H), 1.87-1.82 (m, 1H), 1.23 (s, 6H), 1.05-1.03 (m, 2H), 0.96-0.93 (m, 4H), 0.69-0.68 (m, 2H).

### Example 288: 4-cyclopropyl-7-(2-((2-ethyl-4-(1-(2,2,2-trifluoroethyl)piperidin-4-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 281, where 2,2-difluoroethyl trifluoromethanesulfonate was replaced with 2,2,2-trifluoroethyl trifluoromethanesulfonate in step 2. The title compound was isolated in 27% yield. MS (ESI) m/z: 688.3 [M+H]⁺. ¹H NMR (400 MHz, methanol-d₄) δ 8.66 (s, 1H), 7.96 (s, 1H), 7.33 (s, 1H), 7.20 (s, 1H), 7.14 (dd, J=8.0, 1.6 Hz, 1H), 4.58 (s, 1H), 3.94 (d, J=4.8 Hz, 2H), 3.75 (t, J=5.6 Hz, 2H), 3.10 (m, 4H), 2.92-2.87 (m, 1H), 2.66 (q, J=14.8, 7.2 Hz, 2H), 2.57-2.48 (m, 3H), 1.86-1.82 (m, 4H), 1.18 (t, J=7.6 Hz, 3H), 0.91-0.89 (m, 4H).

### Example 289: 4-cyclopropyl-7-(2-((7-cyclopropyl-2-(oxetan-3-yl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 4-cyclopropyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and oxetan-3-one. The title compound was isolated in 65% yield. m/z (ESI, +ve)=646.2 [M+H]⁺. ¹H NMR (400 MHz, DMSO-d₆) δ 9.90 (s, 1H), 8.89-8.74 (m, 1H), 7.80 (s, 1H), 7.27-7.10 (m, 1H), 6.70 (s, 1H), 4.68-4.59 (m, 2H), 4.58-4.50 (m, 2H), 3.96-3.76 (m, 5H), 3.65-3.48 (m, 4H), 2.91-2.76 (m, 3H), 2.01-1.84 (m, 1H), 0.86-0.74 (m, 6H), 0.61-0.49 (m, 2H).

### Example 290: 7-(2-((7-cyclopropyl-2-(2-fluoroethyl)-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 227, where 7-(2-((7-chloro-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((7-cyclopropyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 40% yield. MS (ESI) m/z: 651.1 [M+H]⁺. ¹H NMR (400 MHz, CDCl₃-d) δ, 0.65-0.70 (m, 2H) 0.98-1.05 (m, 2H) 1.79-1.86 (m, 1H) 2.87 (br t, J=5.6 Hz, 3H) 2.93 (t, J=4.8 Hz, 1H) 3.00 (br t, J=5.2 Hz, 2H) 3.71 (s, 2H) 3.77-3.82 (m, 2H) 4.14 (t, J=5.6 Hz, 2H) 4.59-4.65 (m, 1H) 4.71-4.77 (m, 3H) 5.04 (t, J=7.6 Hz, 2H) 5.56 (t, J=6.4 Hz, 1H) 6.86 (s, 1H) 7.85 (br s, 1H) 7.90 (br s, 1H) 8.12 (s, 1H) 8.70-8.79 (m, 1H).

### Example 291: 7-(2-((7-cyclopropyl-2-(2-hydroxy-2-methylpropyl)-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

To a solution of 7-(2-((7-cyclopropyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide (0.07 mmol) and 2,2-dimethyloxirane (0.07 mmol) in ethanol (3 mL) was added triethylamine (0.33 mmol).and the mixture was stirred at 80° C. for 12 hours. The reaction was concentrated under reduced pressure to afford a residue that was purified by preparative TLC (10% methanol in dichloromethane) to afford the title compound in 53% yield. MS (ESI) m/z: 678.1 [M+H]⁺. ¹H NMR (400 MHz, CDCl₃) δ 8.76 (s, 1H), 8.13 (s, 1H), 8.08-7.95 (m, 1H), 7.92-7.74 (m, 1H), 6.87 (s, 1H), 5.57-5.49 (m, 1H), 5.04 (t, J=7.6 Hz, 2H), 4.74 (t, J=6.8 Hz, 2H), 4.15 (t, J=6.0 Hz, 2H), 3.79 (t, J=6.0 Hz, 4H), 3.29-2.89 (m, 4H), 2.87-2.39 (m, 2H), 1.86-1.81 (m, 1H), 1.37-1.25 (m, 6H), 1.06 (d, J=7.6 Hz, 2H), 0.72-0.66 (m, 2H).

### Example 292: 7-(2-((7-cyclopropyl-2-(oxetan-3-yl)-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 17, where 5-(2-((6-chloroisoindolin-5-yl)amino)-5-(trifluoromethyl)pyrimidin-4-yl)thiophene-3-carboxamide and formaldehyde were replaced with 7-(2-((7-cyclopropyl-2-(oxetan-3-yl)-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide and oxetan-3-one. The title compound was isolated in 49% yield. m/z (ESI, +ve)=662.4 [M+H]⁺. ¹H NMR (400 MHz, CDCl₃) δ 8.75 (s, 1H), 8.12 (s, 1H), 7.93 (s, 1H), 7.85 (br s, 1H), 6.85 (s, 1H), 5.60-5.48 (m, 1H), 5.03 (t, J=7.6 Hz, 2H), 4.80-4.71 (m, 6H), 4.14 (t, J=6.0 Hz, 2H), 3.79 (t, J=5.6 Hz, 2H), 3.70 (t, J=6.4 Hz, 1H), 3.48 (s, 2H), 3.00 (br t, J=5.6 Hz, 2H), 2.64 (t, J=6.0 Hz, 2H), 1.86-1.79 (m, 1H), 1.05-1.00 (m, 2H), 0.70-0.63 (m, 2H).

### Example 293: 4-Cyclopropyl-7-(2-((2,7-dicyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

To a solution of 4-cyclopropyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide (0.07 mmol) in methanol (0.5 mL) and tetrahydrofuran (0.5 mL) was added (1-ethoxycyclopropoxy)trimethylsilane (0.14 mmol), acetic acid (0.1 mL) and sodium cyanoborohydride (0.10 mmol). The mixture was stirred at 80° C. for 12 hours, cooled down to room temperature and purified by preparative TLC (9% methanol in dichloromethane) to afford the title compound in 50% yield. MS (ESI) m/z: 630.1 [M+H]⁺. ¹H NMR (400 MHz, CDCl₃) δ=8.73 (s, 1H), 8.08 (s, 1H), 8.01-7.92 (m, 1H), 7.91-7.83 (m, 1H), 6.88 (s, 1H), 3.96 (t, J=6.0 Hz, 2H), 3.91-3.70 (m, 2H), 3.65 (t, J=6.0 Hz, 2H), 3.15-2.95 (m, 2H), 2.95-2.90 (m, 1H), 1.90-1.75 (m, 2H), 1.26 (s, 2H), 1.08-1.00 (m, 2H), 1.00-0.84 (m, 5H), 0.70-0.66 (m, 2H), 0.65-0.44 (m, 3H).

### Example 294: 4-Cyclopropyl-7-(2-((4-(4-cyclopropylpiperazin-1-yl)-2-ethylphenyl)amino)-5-(trifluoromethyl)pyrimidin-4-yl)-3,4-dihydrothieno[2,3-f][1,4]thiazepin-5(2H)-one 1,1-dioxide

A solution of 4-cyclopropyl-7-(2-((2-ethyl-4-(piperazin-1-yl)phenyl)amino)-5-(trifluoromethyl)pyrimidin-4-yl)-3,4-dihydrothieno[2,3-f][1,4]thiazepin-5(2H)-one 1,1-dioxide (0.07 mmol) and cyclopropylboronic acid (0.07 mmol) in dichloroethane (2 mL) was treated with sodium carbonate (0.19 mmol), 2-(2-pyridyl)pyridine (0.13 mmol) and copper acetate (0.13 mmol). The mixture was stirred at 60° C. for 2 hours, cooled down to room temperature, filtered and concentrated under reduced pressure to afford a residue that was purified by preparative HPLC. The title compound was isolated in 27% yield. MS (ESI) m/z: 647.3 [M+H]⁺. ¹H NMR (400 MHz, CDCl₃) δ ppm 0.39-0.61 (m, 3H) 0.75-0.97 (m, 4H) 1.05-1.24 (m, 4H) 1.40-1.59 (m, 2H) 2.51-2.60 (m, 2H) 2.67-2.88 (m, 4H) 3.05-3.27 (m, 4H) 3.47-3.60 (m, 2H) 3.79-3.92 (m, 2H) 6.67-6.84 (m, 2H) 6.89-7.09 (m, 1H) 7.31-7.67 (m, 1H) 7.87-8.03 (m, 1H) 8.49-8.69 (m, 1H).

### Example 295: 7-(2-((7-chloro-1,2,3,4-tetrahydroisoquinolin-6-yl)amino)-5-(trifluoromethyl)pyrimidin-4-yl)-3,4-dihydro-2H-thieno[3,2-f][1,5,2]dithiazepine 1,1,5,5-tetraoxide

### Step 1: 3-Bromothiophene-2-sulfonyl chloride

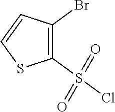

A 0° C. solution of 3-bromothiophene (123 mmol) in dichloromethane (200 mL) was treated with a solution of sulfurochloridic acid (491 mmol) in dichloromethane (200 mL). The reaction was allowed to reach 25° C. over 30 minutes and stirring continued for another 4 hours. The mixture was poured into ice water and extracted with dichloromethane twice. The combined organic layers were washed with brine, dried over anhydrous sodium sulfate, filtered, and concentrated under reduced pressure to afford a residue that was purified by silica gel column chromatography (0-15% ethyl acetate in hexanes). The title compound was isolated as a colorless oil in 57% yield. ¹H NMR (400 MHz, CDCl₃) δ 7.74 (d, J=5.2 Hz, 1H), 7.23 (d, J=5.2 Hz, 1H).

### Step 2: 3-Bromo-N-(4-methoxybenzyl)thiophene-2-sulfonamide

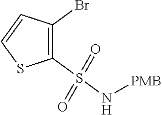

To a 0° C. solution of 3-bromothiophene-2-sulfonyl chloride (57 mmol) in dichloromethane (150 mL) was added (4-methoxyphenyl)methanamine (68 mmol) and triethylamine (68 mmol). The mixture was stirred at room temperature for 30 minutes and concentrated under reduced pressure to afford a residue that was purified by silica gel column chromatography (5-25% ethyl acetate in hexanes). The title compound was isolated in 91% yield. ¹H NMR (400 MHz, CDCl₃) δ 7.51 (d, J=5.2 Hz, 1H), 7.19-7.13 (m, 2H), 7.10 (d, J=5.2 Hz, 1H), 6.87-6.80 (m, 2H), 5.18 (t, J=5.6 Hz, 1H), 4.15 (d, J=5.6 Hz, 2H), 3.80 (s, 3H).

### Step 3: 3-((2-Hydroxyethyl)thio)-N-(4-methoxybenzyl)thiophene-2-sulfonamide

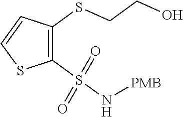

To a solution of 3-bromo-N-(4-methoxybenzyl)thiophene-2-sulfonamide (51 mmol) and 2-mercaptoethan-1-ol (77 mmol) in dioxane (200 mL) was added N,N-diisopropylethyl amine (154 mmol) and (1,1′-bis(diphenylphosphino)ferrocene)palladium(II) dichloride (1.28 mmol). The mixture was stirred at 130° C., cooled down to room temperature and diluted with water. The mixture was extracted with ethyl acetate twice, the combined organic layers were washed with brine, dried over anhydrous sodium sulfate, filtered, and concentrated under vacuum to afford a residue that was purified by preparative TLC (35% ethyl acetate in hexanes). The title compound was isolate din 79% yield. ¹H NMR (400 MHz, CDCl₃) δ 7.54 (d, J=5.2 Hz, 1H), 7.17-7.09 (m, 3H), 6.82 (d, J=8.4 Hz, 2H), 5.50 (t, J=5.6 Hz, 1H), 4.13-4.10 (m, 2H), 3.79 (s, 3H), 3.65 (q, J=5.6 Hz, 2H), 3.06 (t, J=5.6 Hz, 2H), 2.65 (t, J=6.0 Hz, 1H).

### Step 4: 2-(4-Methoxybenzyl)-3,4-dihydro-2H-thieno[3,2-f][1,5,2]dithiazepine 1,1-dioxide

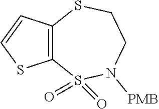

To a solution of 3-((2-hydroxyethyl)thio)-N-(4-methoxybenzyl)thiophene-2-sulfonamide (41 mmol) in tetrahydrofuran (150 mL) was added triphenylphosphine (49 mmol) and diisopropylazodicarboxylate (49 mmol). The mixture was stirred at 25° C. for 30 minutes, diluted with water and extracted with ethyl acetate three times. The organic layers were combined and washed with brine, dried over anhydrous sodium sulfate, filtered, and concentrated under reduced pressure to afford a residue that was purified by silica gel column chromatography (0-25% ethyl acetate in hexanes). The title compound was isolated in 79% yield. ¹H NMR (400 MHz, CDCl₃) δ 7.36 (d, J=5.2 Hz, 1H), 7.21-7.19 (m, 2H), 6.95 (d, J=5.2 Hz, 1H), 6.85-6.78 (m, 2H), 4.91 (td, J=6.4, 12.4 Hz, 4H), 4.23 (s, 2H), 3.74 (s, 3H), 3.71 (td, J=2.4, 5.2 Hz, 2H), 2.96-2.89 (m, 2H).

### Step 5: 7-Iodo-2-(4-methoxybenzyl)-3,4-dihydro-2H-thieno[3,2-f][1,5,2]dithiazepine 1,1-dioxide

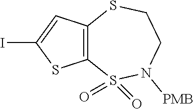

To a −40° C. solution of 2-(4-methoxybenzyl)-3,4-dihydro-2H-thieno[3,2-f][1,5,2]dithiazepine 1,1-dioxide (0.59 mmol) in tetrahydrofuran (5 mL) was added a 1.5 M solution of 2,2,6,6-tetramethylpiperidinylmagnesium chloride lithium chloride complex in THF/toluene (1.56 mL). After 30 minutes at room temperature, the mixture was cooled down to −70° C. and iodine (1.17 mmol) in tetrahydrofuran (5 mL) was added dropwise. After 1 hour at room temperature the reaction mixture was poured into water, extracted with ethyl acetate twice and the combined organic layers were washed with brine, dried over anhydrous sodium sulfate, filtered, and concentrated under reduced pressure to afford a residue that was purified by preparative TLC (25% ethyl acetate in hexanes). The title compound was isolated in 37% yield as a colorless oil. ¹H NMR (400 MHz, CDCl₃) δ 7.27-7.23 (m, 2H), 7.18 (s, 1H), 6.92-6.87 (m, 2H), 4.31 (s, 2H), 3.82 (s, 3H), 3.76 (td, J=2.4, 5.2 Hz, 2H), 3.04-2.94 (m, 2H).

### Step 6: 7-Iodo-3,4-dihydro-2H-thieno[3,2-f][1,5,2]dithiazepine 1,1-dioxide

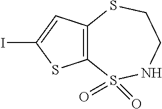 [SMILES: O=S1(=O)NCCSc2cc(I)sc21]

Trifluoroacetic acid (2 mL) was added to a solution of 7-iodo-2-(4-methoxybenzyl)-3,4-dihydro-2H-thieno[3,2-f][1,5,2]dithiazepine 1,1-dioxide (0.14 mmol) in dichloromethane (2 mL). After 2 hours the reaction was concentrated under reduced pressure and the residue was purified by preparative TLC (50% ethyl acetate in hexanes) to afford the title compound. ¹H NMR (400 MHz, DMSO-d₆) δ 8.26 (s, 1H), 7.35 (s, 1H), 3.61 (s, 2H), 2.98 (td, J=2.4, 4.8 Hz, 2H).

### Step 7: 7-Iodo-3,4-dihydro-2H-thieno[3,2-f][1,5,2]dithiazepine 1,1,5,5-tetraoxide

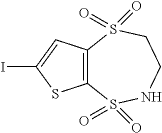 [SMILES: O=S1(=O)CCNS(=O)(=O)c2sc(I)cc21]

A 0° C. solution of 7-iodo-3,4-dihydro-2H-thieno[3,2-f][1,5,2]dithiazepine 1,1-dioxide (0.14 mmol) in dichloromethane (1 mL) was treated with meta-chloroperoxybenzoic acid (0.30 mmol). After 30 minutes the reaction was quenched with water, extracted with ethyl acetate three times and the combined organic layers were dried over anhydrous sodium sulfate, filtered, and concentrated under reduced pressure to afford a residue that was purified by silica gel chromatography (50% ethyl acetate in hexanes). The title compound was isolated in 59% yield. ¹H NMR (400 MHz, DMSO-d₆) δ 7.88 (s, 1H), 7.74 (s, 1H), 3.80 (d, J=4.4 Hz, 2H), 3.73-3.65 (m, 2H).

### Steps 8-9: 7-(2-((7-chloro-1,2,3,4-tetrahydroisoquinolin-6-yl)amino)-5-(trifluoromethyl)pyrimidin-4-yl)-3,4-dihydro-2H-thieno[3,2-f][1,5,2]dithiazepine 1,1,5,5-tetraoxide

The title compound was prepared analogously to Example 155, steps 3-4, where 7-bromo-4-cyclopropyl-3,4-dihydrothieno[2,3-f][1,4]thiazepin-5(2H)-one 1,1-dioxide were replaced with 7-iodo-3,4-dihydro-2H-thieno[3,2-f][1,5,2]dithiazepine 1,1,5,5-tetraoxide in step 3. The title compound was isolated. MS (ESI) m/z: 580.0 [M+H]⁺. ¹H NMR (400 MHz, DMSO-d₆) δ 10.08 (d, J=1.2 Hz, 1H), 8.92-8.90 (m, 1H), 7.90 (s, 1H), 7.41-7.33 (m, 1H), 7.29 (s, 1H), 3.95 (s, 2H), 3.79 (d, J=4.4 Hz, 2H), 3.69-3.65 (m, 2H), 3.05 (t, J=5.2 Hz, 2H), 2.79-2.74 (m, 2H).

### Example 296: 7-(2-((4-(1-(2,2-difluoroethyl)piperidin-4-yl)-2-ethylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 281, step 2, where 4-cyclopropyl-7-[2-[2-ethyl-4-(4-piperidyl)anilino]-5-(trifluoromethyl)pyrimidin-4-yl]-1,1-dioxo-2,3-dihydrothieno[2,3-f][1,4]thiazepin-5-one was replaced with 7-(2-((2-ethyl-4-(piperidin-4-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 19% yield. MS (ESI) m/z: 686.3[M+H]⁺. ¹H NMR (400 MHz, DMSO-d₆) δ 9.83 (s, 1H), 8.78 (bs, 1H), 7.82 (s, 1H), 7.25-7.22 (m, 1H), 7.17 (d, J=1.6 Hz, 1H), 7.11-7.08 (m, 1H), 6.15 (tt, J=55.6, 4.4 Hz, 1H), 5.24-5.21 (m, 1H), 4.75-4.68 (m, 4H), 4.02 (s, 4H), 3.01 (d, J=11.2 Hz, 2H), 2.75 (td, J=15.6, 4.0 Hz, 2H), 2.61-2.57 (m, 2H), 2.33-2.24 (m, 3H), 1.76-1.65 (m, 4H), 1.10 (t, J=7.6 Hz, 3H).

### Example 297: 4-Cyclopropyl-7-(2-((7-cyclopropyl-2-((4,4-dimethyloxetan-2-yl)methyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 227, where 7-(2-((7-chloro-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide and 1-bromo-2-fluoroethane were replaced with 4-cyclopropyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and (4,4-dimethyloxetan-2-yl)methyl 4-methylbenzenesulfonate. The title compound was isolated in 16% yield. MS (ESI) m/z: 688.1 [M+H]⁺. ¹H NMR (400 MHz, CDCl₃) δ=8.74 (s, 1H), 8.08 (s, 1H), 8.04-7.95 (m, 1H), 7.88 (s, 1H), 6.88 (s, 1H), 5.18-4.80 (m, 1H), 4.07-3.93 (m, 2H), 3.89-3.53 (m, 4H), 3.31-2.78 (m, 7H), 2.61-2.45 (m, 1H), 2.29-2.14 (m, 1H), 1.87-1.79 (m, 1H), 1.49 (s, 3H), 1.42 (s, 3H), 1.07-1.01 (m, 2H), 1.00-0.93 (m, 4H), 0.72-0.64 (m, 2H).

### Example 298: 7-(2-((2-Ethyl-4-(1-(2,2,2-trifluoroethyl)piperidin-4-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

### Step 1: Methyl 3-iodothiophene-2-carboxylate

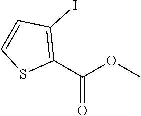

To a solution of methyl 3-aminothiophene-2-carboxylate (118 mmol) in acetonitrile (400 mL) was added diiodomethane (353 mmol) and tert-butyl nitrite (177 mmol). The reaction mixture was stirred at 50° C. for 1 hour, cooled down to room temperature, quenched with aqueous sodium sulfite and concentrated under vacuum. The aqueous phase was extracted with ethyl acetate three times, the combined organic layers were dried over anhydrous sodium sulfate, filtered, and concentrated under reduced pressure to afford a residue that was purified by silica gel chromatography (1% ethyl acetate in hexanes) to afford the title compound in 99% yield. ¹H NMR (400 MHz, CDCl₃) δ=7.45 (d, J=5.2 Hz, 1H), 7.24 (d, J=5.2 Hz, 1H), 3.91 (s, 3H).

### Step 2: Methyl 3-((2-hydroxyethyl)thio)thiophene-2-carboxylate

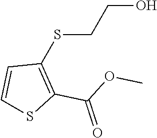

A mixture of methyl 3-iodothiophene-2-carboxylate (120 mmol), 2-mercaptoethan-1-ol (139 mmol), tris(dibenzylidenacetone)dipalladium (12 mmol), (9,9-dimethyl-9H-xanthene-3,5-diyl)bis(diphenylphosphine) (12 mmol) and N,N′-diisopropylethylamine (361 mmol) in dioxane (350 mL) was stirred at 130° C. for 4 hours. The resulting mixture was cooled down to room temperature, filtered and the filtrate was concentrated under vacuum. The residue was purified by silica gel column chromatography (5-40% ethyl acetate in hexanes) to afford the title compound in 65% yield. MS (ESI) m/z: 219.0 [M+H]⁺.

### Step 3: methyl 3-((2-((tert-butyldimethylsilyl)oxy)ethyl)thio)thiophene-2-carboxylate

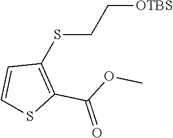 [SMILES: COC(=O)c1sccc1SCCO[Si](C)(C)C(C)(C)C]

To a 0° C. solution of methyl 3-((2-hydroxyethyl)thio)thiophene-2-carboxylate (78 mmol) and imidazole (235 mmol) in dichloromethane (200 mL) was added tert-butylchlorodimethylsilane (118 mmol). The reaction mixture was stirred at room temperature for 12 hours, quenched with water and extracted with dichloromethane twice. The combined organic layers were washed with brine, dried over anhydrous sodium sulfate, filtered, and concentrated under reduced pressure to afford a residue that was purified by silica gel chromatography (0-3% ethyl acetate in hexanes). The title compound was isolated in 99% yield. MS (ESI) m/z: 333.1 [M+Na]+. ¹H NMR (400 MHz, CDCl₃) δ 7.49 (d, J=5.2 Hz, 1H), 7.07 (d, J=5.2 Hz, 1H), 3.95-3.76 (m, 5H), 3.17 (t, J=7.2 Hz, 2H), 0.90 (s, 9H), 0.07 (s, 6H).

### Step 4: Methyl 3-((2-((tert-butyldimethylsilyl)oxy)ethyl)thio)-5-iodothiophene-2-carboxylate

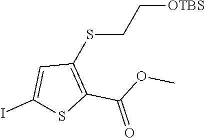 [SMILES: COC(=O)c1sc(I)cc1SCCO[Si](C)(C)C(C)(C)C]

To a −40° C. solution of methyl 3-((2-((tert-butyldimethylsilyl)oxy)ethyl)thio)thiophene-2-carboxylate (75 mmol) in tetrahydrofuran (300 mL) was added a 1.5M solution of lithium chloro-(2,2,6,6-tetramethyl-1-piperidyl)magnesium chloride in THF (150 mL) and the reaction was stirred at this temperature for 30 minutes. A 1M THF solution of iodine (150 mmol) was added dropwise and the reaction was stirred at room temperature for 1 hour. The reaction mixture was poured into water and extracted with ethyl acetate twice. The combined organic layers were washed with brine, dried over anhydrous sodium sulfate, filtered, and concentrated under reduced pressure to afford a residue that was purified by silica gel chromatography (1-5% ethyl acetate in hexanes) to afford the title compound in 81% yield. MS (ESI) m/z: 459.0 [M+H]⁺. ¹H NMR (400 MHz, CDCl₃) δ 7.22 (s, 1H), 3.91-3.76 (m, 5H), 3.14 (t, J=6.8 Hz, 2H), 0.90 (s, 9H), 0.08 (s, 6H).

### Step 5: Methyl 3-((2-hydroxyethyl)thio)-5-iodothiophene-2-carboxylate

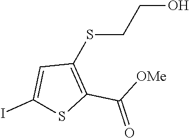 [SMILES: COC(=O)c1sc(I)cc1SCCO]

To a solution of methyl 3-((2-((tert-butyldimethylsilyl)oxy)ethyl)thio)-5-iodothiophene-2-carboxylate (61 mmol) in THF (200 mL) was added a 1M solution of tetrabutylammonium fluoride in THF (40 mL). After 2 hours, the mixture was diluted with ethyl acetate and washed with water, dried over anhydrous sodium sulfate, filtered, and concentrated under reduced pressure to afford a residue that was purified by silica gel chromatography (5-30% ethyl acetate in hexanes). The title compound was isolated in 55% yield. MS (ESI) m/z: 344.8 [M+H]⁺. ¹H NMR (400 MHz, methanol-d₄) δ 7.35 (s, 1H), 3.82 (s, 3H), 3.76 (t, J=6.8 Hz, 2H), 3.16 (t, J=6.8 Hz, 2H).

### Step 6: Methyl 3-((2-bromoethyl)thio)-5-iodothiophene-2-carboxylate

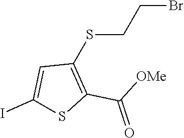 [SMILES: COC(=O)c1sc(I)cc1SCCBr]

To a 0° C. solution of methyl 3-((2-hydroxyethyl)thio)-5-iodothiophene-2-carboxylate (15 mmol) in dichloromethane (60 mL) was added carbon tetrabromide (22 mmol) and triphenylphosphine (22 mmol). The reaction mixture was stirred at 45° C. for 1 hour, concentrated under reduced pressure and the resulting residue was purified by silica gel chromatography (3-10% ethyl acetate in hexanes) to afford the title compound in 88% yield. ¹H NMR (400 MHz, methanol-d₄) δ=7.32 (s, 1H), 3.83 (s, 3H), 3.64-3.56 (m, 2H), 3.53-3.45 (m, 2H).

### Step 7: Methyl 3-((2-(cyclopropylamino)ethyl)thio)-5-iodothiophene-2-carboxylate

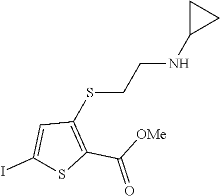 [SMILES: COC(=O)c1sc(I)cc1SCCNC1CC1]

To a solution of methyl 3-((2-bromoethyl)thio)-5-iodothiophene-2-carboxylate (491 mmol) and cyclopropanamine (0.98 mmol) in acetonitrile (2 mL) was added potassium carbonate (1.47 mmol). The reaction mixture was stirred at 80° C. for 12 hours, cooled down to room temperature and filtered. The filtrate was concentrated under vacuum and the residue was purified by preparative TLC (50% ethyl acetate in hexanes). The title compound was isolated in 32% yield. MS (ESI) m/z: 383.9 [M+H]⁺. ¹H NMR (400 MHz, methanol-d₄) δ 7.38 (s, 1H), 3.83 (s, 3H), 3.20-3.12 (m, 2H), 3.02-2.84 (m, 2H), 2.23-2.20 (m, 1H), 0.59-0.46 (m, 2H), 0.45-0.26 (m, 2H).

### Step 8: 3-((2-(Cyclopropylamino)ethyl)thio)-5-iodothiophene-2-carboxylic acid

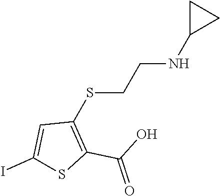 [SMILES: O=C(O)c1sc(I)cc1SCCNC1CC1]

Lithium hydroxide monohydrate (3.4 mmol) was added to a solution of methyl 3-((2-(cyclopropylamino)ethyl)thio)-5-iodothiophene-2-carboxylate (2.27 mmol) in methanol (10 mL). The reaction mixture was stirred at room temperature for 12 hours and the volatiles removed under reduced pressure to afford the title compound, which was used in the next step without further purification. MS (ESI) m/z: 370.1 [M+H]⁺.

### Step 9: 4-Cyclopropyl-7-iodo-3,4-dihydrothieno[2,3-f][1,4]thiazepin-5(2H)-one

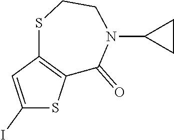 [SMILES: O=C1c2sc(I)cc2SCCN1C1CC1]

A solution of 3-((2-(cyclopropylamino)ethyl)thio)-5-iodothiophene-2-carboxylic acid (2.20 mmol) and HATU (3.29 mmol) in DMF (10 mL) was treated with N,N′-diisopropylethylamine (6.6 mmol). After 1 hour, the reaction was quenched with water and extracted with ethyl acetate three times. The combined organic layers were washed with brine, dried over anhydrous sodium sulfate, filtered, and concentrated under reduced pressure to afford a residue that was purified by silica gel chromatography (5-20% ethyl acetate in hexanes) to afford the title compound in 61% yield. MS (ESI) m/z: 352.1 [M+H]⁺.

### Step 10: 4-Cyclopropyl-7-iodo-3,4-dihydrothieno[2,3-f][1,4]thiazepin-5(2H)-one 1,1-dioxide

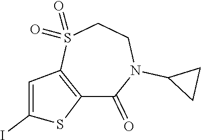 [SMILES: O=C1c2sc(I)cc2S(=O)(=O)CCN1C1CC1]

To a 0° C. solution of 4-cyclopropyl-7-iodo-3,4-dihydrothieno[2,3-f][1,4]thiazepin-5(2H)-one (0.071 mmol) in dichloromethane (1 mL) was added 85% 3-chloroperbenzoic acid (0.21 mmol). After 12 hours, the reaction was quenched with water and extracted with dichloromethane twice. The combined organic layers were washed with saturated sodium sulfite, dried over anhydrous sodium sulfate, filtered, and concentrated under reduced pressure to afford a residue that was purified by preparative TLC (33% ethyl acetate in hexanes). The title compound was isolated in 73% yield. MS (ESI) m/z: 383.9 [M+H]⁺

¹H NMR (400 MHz, methanol-d₄) δ 7.61 (s, 1H), 4.02-3.93 (m, 2H), 3.79-3.72 (m, 2H), 2.91-2.87 (m, 1H), 0.92-0.87 (m, 4H).

### Steps 11-14: 7-(2-((2-Ethyl-4-(piperidin-4-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

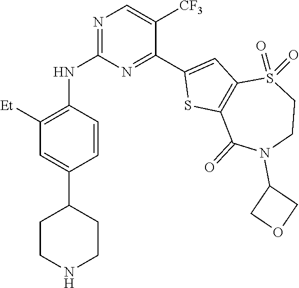 [SMILES: CCc1cc(C2CCNCC2)ccc1Nc1ncc(C(F)(F)F)c(-c2cc3c(s2)C(=O)N(C2COC2)CCS3(=O)=O)n1]

The title compound was prepared analogously to Example 240, steps 2-5, where tert-butyl 4-(4-((4-chloro-5-(trifluoromethyl)pyrimidin-2-yl)amino)-3-cyclopropylphenyl)piperidine-1-carboxylate was replaced with tert-butyl 4-(4-((4-chloro-5-(trifluoromethyl)pyrimidin-2-yl)amino)-3-ethyllphenyl)piperidine-1-carboxylate in step 2 and 7-bromo-4-methyl-3,4-dihydrothieno[2,3-f][1,4]thiazepin-5(2H)-one 1,1-dioxide was replaced with 7-iodo-4-(oxetan-3-yl)-3,4-dihydrothieno[2,3-f][1,4]thiazepin-5(2H)-one 1,1-dioxide in step 4. The title compound was isolated. MS (ESI) m/z: 622.2[M+H]⁺.

### Step 15: 7-(2-((2-ethyl-4-(1-(2,2,2-trifluoroethyl)piperidin-4-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 281, where 4-cyclopropyl-7-[2-[2-ethyl-4-(4-piperidyl)anilino]-5-(trifluoromethyl)pyrimidin-4-yl]-1,1-dioxo-2,3-dihydrothieno[2,3-f][1,4]thiazepin-5-one and 2,2-difluoroethyl trifluoromethanesulfonate were replaced with 7-(2-((2-ethyl-4-(piperidin-4-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide and 2,2,2-trifluoroethyl trifluoromethanesulfonate. The title compound was isolated in 40% yield. MS (ESI) m z: 704.3[M+H]⁺. ¹H NMR (400 MHz, DMSO-d₆) δ 9.84 (s, 1H), 8.79 (s, 1H), 7.82 (s, 1H), 7.25 (s, 1H), 7.17 (s, 1H), 7.10 (d, J=7.6 Hz, 1H), 5.22 (t, J=6.8 Hz, 1H), 4.75-4.67 (m, 4H), 4.01 (d, J=4.0 Hz, 4H), 3.20 (q, J=20.4, 10.0 Hz, 2H), 3.02 (d, J=11.6 Hz, 2H), 2.59-2.55 (m, 2H), 2.46-2.43 (m, 3H), 1.76-1.66 (m, 4H), 1.10 (t, J=7.2 Hz, 3H).

### Example 299: 4-cyclopropyl-7-(2-((7-cyclopropyl-2-(2-fluoro-2-methylpropyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 227, where 7-(2-((7-chloro-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide and 1-bromo-2-fluoroethane were replaced with 4-cyclopropyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and 2-fluoro-2-methylpropyl trifluoromethanesulfonate. The title compound was isolated in 36% yield. MS (ESI) m/z: 664.2 [M+H]⁺. ¹H NMR (400 MHz, DMSO-d₆) δ 9.86 (s, 1H), 8.80 (br s, 1H), 7.80 (s, 1H), 7.16 (brs, 1H), 6.66 (s, 1H), 3.91-3.82 (m, 4H), 3.65-3.60 (m, 2H), 2.90-2.84 (m, 1H), 2.78-2.76 (m, 2H), 2.65-2.62 (m, 1H), 2.59-2.57 (m, 1H), 2.08-2.05 (m, 2H), 1.96-1.88 (m, 1H), 1.34 (d, J=21.6 Hz, 6H), 0.83-0.76 (m, 3H), 0.59-0.53 (m, 2H).

### Example 300: 7-(2-((7-cyclopropyl-2-(2-fluoro-2-methylpropyl)-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 227, where 7-(2-((7-chloro-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide and 1-bromo-2-fluoroethane were replaced with 7-(2-((7-cyclopropyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide and 2-fluoro-2-methylpropyl trifluoromethanesulfonate. The title compound was isolated in 56% yield. MS (ESI) m/z: 680.1 [M+H]⁺. ¹H NMR (400 MHz, CDCl₃) δ 8.75 (s, 1H), 8.12 (s, 1H), 8.09-7.73 (m, 2H), 6.86 (s, 1H), 5.55 (t, J=6.8 Hz, 1H), 5.04 (t, J=7.2 Hz, 2H), 4.74 (t, J=6.8 Hz, 2H), 4.14 (br t, J=5.6 Hz, 2H), 3.79 (br t, J=6.0 Hz, 4H), 3.28-2.88 (m, 4H), 2.87-2.57 (m, 2H), 1.86-1.79 (m, 1H), 1.50-1.46 (m, 3H), 1.42 (br d, J=1.2 Hz, 3H), 1.05-1.00 (m, 2H), 0.68 (br d, J=4.8 Hz, 2H).

### Example 301: 7-(2-((2,7-dicyclopropyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 293, where 4-cyclopropyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide was replaced with 7-(2-((7-cyclopropyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 41% yield. MS (ESI) m z: 646.4 [M+H]⁺. ¹H NMR (400 MHz, CDCl₃) δ 8.74 (s, 1H), 8.11 (s, 1H), 7.98-7.77 (m, 2H), 6.87 (s, 1H), 5.63-5.47 (m, 1H), 5.04 (t, J=7.4 Hz, 2H), 4.78-4.70 (m, 4H), 4.13 (t, J=5.8 Hz, 2H), 3.86-3.75 (m, 4H), 3.03-2.96 (m, 3H), 1.85-1.80 (m, 1H), 1.05-1.00 (m, 2H), 0.71-0.66 (m, 2H), 0.66-0.54 (m, 4H).

### Example 302: 7-(2-((7-chloro-1,2,3,4-tetrahydroisoquinolin-6-yl)amino)-5-(trifluoromethyl)pyrimidin-4-yl)-2-methyl-3,4-dihydro-2H-thieno[3,2-f][1,5,2]dithiazepine 1,1,5,5-tetraoxide

### Step 1: 7-Iodo-2-methyl-3,4-dihydro-2H-thieno[3,2-f][1,5,2]dithiazepine 1,1,5,5-tetraoxide

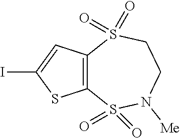 [SMILES: CN1CCS(=O)(=O)c2cc(I)sc2S1(=O)=O]

The title compound was prepared analogously to Example 135, step 1, where 7-bromo-3,4-dihydrothieno[2,3-f][1,4]thiazepin-5(2H)-one 1,1-dioxide and ethyl iodide were replaced with 7-iodo-3,4-dihydro-2H-thieno[3,2-f][1,5,2]dithiazepine 1,1,5,5-tetraoxide and methyl iodide. The title compound was isolated in 39% yield. MS (ESI) m z: 394.9 [M+H]⁺.

### Step 2: 7-(2-((7-chloro-1,2,3,4-tetrahydroisoquinolin-6-yl)amino)-5-(trifluoromethyl)pyrimidin-4-yl)-2-methyl-3,4-dihydro-2H-thieno[3,2-f][1,5,2]dithiazepine 1,1,5,5-tetraoxide

The title compound was prepared analogously to Example 155, steps 3-4, where 7-bromo-4-cyclopropyl-3,4-dihydrothieno[2,3-f][1,4]thiazepin-5(2H)-one 1,1-dioxide was replaced with 7-iodo-2-methyl-3,4-dihydro-2H-thieno[3,2-f][1,5,2]dithiazepine 1,1,5,5-tetraoxide in step 3. The title compound was isolated. MS (ESI) m/z: 594.1 [M+H]⁺. ¹H NMR (400 MHz, DMSO-d₆) δ 10.24-9.97 (m, 1H), 8.97-8.82 (m, 1H), 7.99 (s, 1H), 7.44-7.32 (m, 1H), 7.29 (s, 1H), 4.08 (d, J=2.0 Hz, 2H), 4.01-3.88 (m, 4H), 3.03 (s, 2H), 2.86 (s, 3H), 2.75 (d, J=4.4 Hz, 2H).

### Example 303: 4-cyclopropyl-7-(2-((2-cyclopropyl-5,6,7,8-tetrahydro-1,7-naphthyridin-3-yl)amino)-5-(trifluoromethyl)pyrimidin-4-yl)-3,4-dihydrothieno[2,3-f][1,4]thiazepin-5(2H)-one 1,1-dioxide

### Step 1: tert-butyl 3-amino-2-cyclopropyl-5,8-dihydro-1,7-naphthyridine-7(6H)-carboxylate

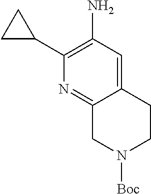 [SMILES: CC(C)(C)OC(=O)N1CCc2cc(N)c(C3CC3)nc2C1]

The title compound was prepared analogously to Example 173, where 1-(7-bromo-6-nitro-3,4-dihydro-1H-isoquinolin-2-yl)-2,2,2-trifluoro-ethanone was replaced with tert-butyl 3-amino-2-bromo-5, 8-dihydro-1, 7-naphthyridine-7(6H)-carboxylate. The title compound was isolated in 81% yield. MS (ESI) m/z: 290.1 [M+H]⁺.

### Steps 2-4: tert-butyl 2-cyclopropyl-3-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)-5,8-dihydro-1,7-naphthyridine-7(6H)-carboxylate

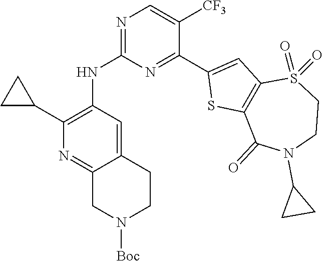 [SMILES: CC(C)(C)OC(=O)N1CCc2cc(Nc3ncc(C(F)(F)F)c(-c4cc5c(s4)C(=O)N(C4CC4)CCS5(=O)=O)n3)c(C3CC3)nc2C1]

The title compound was prepared analogously to Example 137, steps 3-5, where 1-(6-amino-7-ethyl-3,4-dihydroisoquinolin-2(1H)-yl)-2,2,2-trifluoroethan-1-one was replaced with tert-butyl 3-amino-2-cyclopropyl-5,8-dihydro-1,7-naphthyridine-7(6H)-carboxylate in step 3 and 7-bromo-3,4-dihydrothieno[2,3-f][1,4]thiazepin-5(2H)-one 1,1-dioxide was replaced with 7-bromo-4-cyclopropyl-3,4-dihydrothieno[2,3-f][1,4]thiazepin-5(2H)-one 1,1-dioxide in step 5. The title compound was isolated. MS (ESI) m/z: 691.3 [M+H]⁺.

### Step 5: 4-cyclopropyl-7-(2-((2-cyclopropyl-5,6,7,8-tetrahydro-1,7-naphthyridin-3-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 149, step 6, where 7-(2-((2-chloro-5-(piperidin-4-yl)phenyl)amino)-5-(trifluoromethyl)pyrimidin-4-yl)-4-methyl-3,4-dihydrothieno[2,3-f][1,4]thiazepin-5(2H)-one 1,1-dioxide was replaced with tert-butyl 2-cyclopropyl-3-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)-5,8-dihydro-1,7-naphthyridine-7(6H)-carboxylate. The title compound was isolated in 59% yield. MS (ESI) m/z: 591.0 [M+H]⁺. ¹H NMR (400 MHz, CDCl₃-d) δ ppm 0.94 (t, J=4.4 Hz, 2H) 0.95-0.99 (m, 2H) 1.01-1.04 (m, 2H) 1.06 (dt, J=4.4, 2.49 Hz, 2H) 2.02-2.09 (m, 1H) 2.87-2.96 (m, 3H) 3.21 (t, J=6.0 Hz, 2H) 3.65 (t, J=6.4 Hz, 2H) 3.96 (t, J=6.0 Hz, 2H) 4.06 (s, 2H) 7.65 (br s, 1H) 7.99 (br s, 1H) 8.07 (s, 1H) 8.74 (s, 1H).

### Example 304: 7-(2-((2-cyclopropyl-5,6,7,8-tetrahydro-1,7-naphthyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 303 where 7-bromo-4-cyclopropyl-3,4-dihydrothieno[2,3-f][1,4]thiazepin-5(2H)-one 1,1-dioxide was replaced with 7-iodo-4-(oxetan-3-yl)-3,4-dihydrothieno[2,3-f][1,4]thiazepin-5(2H)-one 1,1-dioxide in step 4. The title compound was isolated. MS (ESI) m/z: 607.2 [M+H]⁺. ¹H NMR (400 MHz, CDCl₃-d) δ ppm 1.00-1.08 (m, 4H) 2.01-2.06 (m, 1H) 2.91-2.97 (m, 2H) 3.23 (t, J=5.8 Hz, 2H) 3.79 (t, J=5.8 Hz, 2H) 4.08 (s, 2H) 4.14 (t, J=5.8 Hz, 2H) 4.73 (t, J=6.8 Hz, 2H) 5.03 (t, J=7.6 Hz, 2H) 5.45-5.61 (m, 1H) 7.60 (br s, 1H) 7.96 (s, 1H) 8.11 (s, 1H) 8.76 (s, 1H).

### Example 305: 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide

### Step 1: tert-butyl (1S,4S)-5-(3-cyclopropyl-4-nitrophenyl)-2,5-diazabicyclo[2.2.1]heptane-2-carboxylate

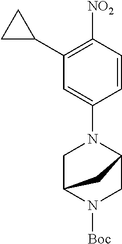 [SMILES: CC(C)(C)OC(=O)N1C[C@@H]2C[C@H]1CN2c1ccc([N+](=O)[O-])c(C2CC2)c1]

Cesium carbonate (166 mmol) was added to a solution of 2-cyclopropyl-4-fluoro-1-nitrobenzene (55 mmol) and tert-butyl (1S,4S)-2,5-diazabicyclo[2.2.1]heptane-2-carboxylate (61 mmol) in DMF (100 mL).

The reaction mixture was stirred at 80° C. for 12 hours, cooled down to room temperature, diluted with water, extracted with ethyl acetate three times and the combined organic layers were dried over anhydrous sodium sulfate, filtered, and concentrated under reduced pressure to afford a residue that was purified by silica gel chromatography (2-15% ethyl acetate in hexanes). The title compound was isolated in 90% yield. MS (ESI) m/z: 360.2 [M+H]⁺. ¹H NMR (400 MHz, methanol-d₄) δ=7.97 (d, J=9.1 Hz, 1H), 6.52 (d, J=8.8 Hz, 1H), 6.29 (s, 1H), 4.68 (s, 1H), 4.59 (s, 1H), 3.60 (d, J=9.8 Hz, 1H), 3.50-3.39 (m, 1H), 3.28 (s, 1H), 3.23 (t, J=8.8 Hz, 1H), 2.62-2.54 (m, 1H), 2.01 (s, 2H), 1.53-1.33 (m, 9H), 1.05-0.95 (m, 2H), 0.70 (t, J=5.5 Hz, 2H).

### Step 2: tert-butyl (1S,4S)-5-(4-amino-3-cyclopropylphenyl)-2,5-diazabicyclo[2.2.1]heptane-2-carboxylate

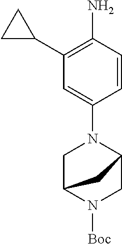 [SMILES: CC(C)(C)OC(=O)N1C[C@@H]2C[C@H]1CN2c1ccc(N)c(C2CC2)c1]

The title compound was prepared analogously to Example 173, step 4, where 1-(7-cyclopropyl-6-nitro-3,4-dihydro-1H-isoquinolin-2-yl)-2,2,2-trifluoro-ethanone was replaced with tert-butyl (1S,4S)-5-(3-cyclopropyl-4-nitrophenyl)-2,5-diazabicyclo[2.2.1]heptane-2-carboxylate. The title compound was isolated in 94% yield. MS (ESI) m/z: 330.3 [M+H]⁺. ¹H NMR (400 MHz, DMSO-d₆) δ 6.88-6.45 (m, 1H), 6.26-6.02 (m, 1H), 5.78-5.48 (m, 1H), 4.67-4.13 (m, 2H), 3.64-3.33 (m, 2H), 3.28-2.93 (m, 2H), 2.07-1.47 (m, 3H), 1.46-1.25 (m, 9H), 0.84 (d, J=4.0 Hz, 2H), 0.54 (s, 2H).

### Step 3: tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate

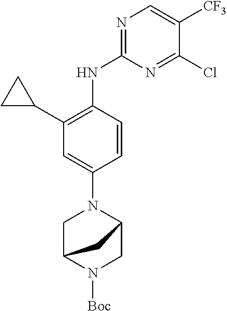 [SMILES: CC(C)(C)OC(=O)N1C[C@@H]2C[C@H]1CN2c1ccc(Nc2ncc(C(F)(F)F)c(Cl)n2)c(C2CC2)c1]

A mixture of tert-butyl (1S,4S)-5-(4-amino-3-cyclopropylphenyl)-2,5-diazabicyclo[2.2.1]heptane-2-carboxylate (46 mmol) and 2,4-dichloro-5-(trifluoromethyl)pyrimidine (232 mmol) was stirred at 70° C. for 12 hours. he resulting mixture was cooled down to room temperature, concentrated and the residue was purified by flash silica gel column chromatography (2-10% ethyl acetate in hexanes) to give an inseparable mixture of two regioisomers. The mixture was further purified by reverse phase preparative HPLC (60-90% acetonitrile in water containing 0.2% formic acid as additive). Most of the acetonitrile was removed under reduced pressure and the pH of the resulting suspension was adjusted to 8-9 with aqueous saturated solution of sodium bicarbonate. The aqueous phase was extracted with ethyl acetate twice, the combined organic layers were washed with brine, dried over anhydrous sodium sulfate, filtered, and concentrated under reduced pressure to afford the title compound in 11% yield. MS (ESI) m/z: 510.4 [M+H]⁺. ¹H NMR (400 MHz, DMSO-d₆) δ 9.89 (s, 1H), 8.75-8.46 (m, 1H), 7.01 (d, J=8.4 Hz, 1H), 6.42 (d, J=8.0 Hz, 1H), 6.08 (s, 1H), 4.57-4.32 (m, 2H), 3.58-3.47 (m, 1H), 3.27 (d, J=11.2 Hz, 1H), 3.21-3.15 (m, 1H), 2.95 (s, 1H), 1.88 (m, 3H), 1.36 (m, 9H), 0.78 (d, J=8.4 Hz, 2H), 0.61 (s, 2H).

### Step 4: tert-butyl (1S,4S)-5-(3-cyclopropyl-4-((5-(trifluoromethyl)-4-(trimethylstannyl)pyrimidin-2-yl)amino)phenyl)-2,5-diazabicyclo[2.2.1]heptane-2-carboxylate

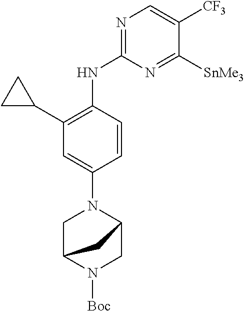

The title compound was prepared analogously to Example 4, step 1, where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate. The purification of this compound was carried out using neutral alumina. The title compound was isolated in 87% yield. MS (ESI) m/z: 640.4 [M+H]⁺.

### Step 5: tert-butyl (1S,4S)-5-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)-2,5-diazabicyclo[2.2.1]heptane-2-carboxylate

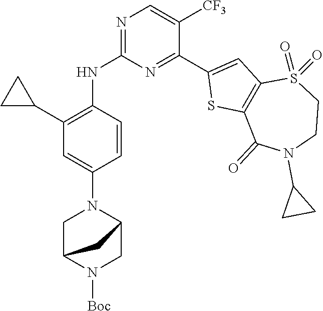 [SMILES: CC(C)(C)OC(=O)N1C[C@@H]2C[C@H]1CN2c1ccc(Nc2ncc(C(F)(F)F)c(-c3cc4c(s3)C(=O)N(C3CC3)CCS4(=O)=O)n2)c(C2CC2)c1]

The title compound was prepared analogously to Example 155, step 3, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one was replaced with tert-butyl (1S,4S)-5-(3-cyclopropyl-4-((5-(trifluoromethyl)-4-(trimethylstannyl)pyrimidin-2-yl)amino)phenyl)-2,5-diazabicyclo[2.2.1]heptane-2-carboxylate. The title compound was isolated in 87% yield. MS (ESI) m/z: 731.5 [M+H]⁺. ¹H NMR (400 MHz, methanol-d₄) δ 8.85-8.43 (m, 1H), 7.96 (s, 1H), 7.37-7.11 (m, 1H), 6.50 (d, J=7.6 Hz, 1H), 6.25 (s, 1H), 4.54-4.43 (m, 2H), 4.05-3.85 (m, 2H), 3.80-3.74 (m, 2H), 3.60 (t, J=7.2 Hz, 1H), 3.44-3.36 (m, 2H), 3.08 (t, J=9.2 Hz, 1H), 2.90-2.85 (s, 1H), 2.12-1.87 (m, 3H), 1.56-1.35 (m, 9H), 1.02-0.79 (m, 6H), 0.64 (t, J=5.9 Hz, 2H).

### Step 6: 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide

To a solution of tert-butyl (1S,4S)-5-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)-2,5-diazabicyclo[2.2.1]heptane-2-carboxylate (0.23 mmol) in dichloromethane (5 mL) was added trifluoroacetic acid (13.5 mmol) and the reaction mixture was stirred at room temperature for 30 minutes. The volatiles were removed under reduced pressure and the residue was diluted with water and the pH of the resulting solution was adjusted to 8-9 by the addition of saturated aqueous solution of sodium hydrogen carbonate. The aqueous phase was extracted with ethyl acetate twice, the combined organic layers were washed with brine, dried over anhydrous sodium sulfate, filtered, and concentrated under reduced pressure to afford the title compound in 95% yield. MS (ESI) m/z: 631.4 [M+H]⁺. ¹H NMR (400 MHz, methanol-d₄) δ 8.84-8.54 (m, 1H), 7.96 (s, 1H), 7.40-7.02 (m, 1H), 6.52 (d, J=7.2 Hz, 1H), 6.27 (s, 1H), 4.72-4.38 (m, 2H), 4.04-3.89 (m, 2H), 3.87-3.70 (m, 3H), 3.62 (m, 1H), 3.10-2.90 (m, 3H), 2.02-1.89 (m, 2H), 1.82 (m, 1H), 0.93-0.84 (m, 6H), 0.71-0.55 (m, 2H).

### Example 306: 4-Cyclopropyl-7-(2-((2-cyclopropyl-4-((1S,4S)-5-methyl-2,5-diazabicyclo[2.2.1]heptan-2-yl)phenyl)amino)-5-(trifluoromethyl)pyrimidin-4-yl)-3,4-dihydrothieno[2,3-f][1,4]thiazepin-5(2H)-one 1,1-dioxide

To a solution of 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide (0.067 mmol), paraformaldehyde (0.67 mmol) and acetic acid (0.007 mmol) in methanol (1 mL) was stirred at room temperature for 30 minutes. Sodium cyanoborohydride (0.20 mmol) was added and the reaction was stirred for two additional hours. The mixture was filtered and the filtrate was concentrated under reduced pressure to afford a residue that was purified by preparative TLC (10% methanol in dichloromethane). The title compound was isolated in 73% yield. MS (ESI) m/z: 645.4 [M+H]⁺. ¹H NMR (400 MHz, methanol-d₄) δ=8.72-8.66 (m, 1H), 7.95 (s, 1H), 7.38-7.20 (m, 1H), 6.68-6.50 (m, 1H), 6.32 (s, 1H), 4.64-4.53 (m, 2H), 3.96-3.90 (m, 2H), 3.77-3.74 (m, 2H), 3.74-3.63 (m, 1H), 3.40-3.32 (m, 3H), 3.27-3.11 (m, 1H), 2.92-2.79 (m, 3H), 2.31-2.21 (m, 1H), 1.94 (s, 2H), 0.91-0.85 (m, 6H), 0.68-0.60 (m, 2H).

### Example 307: 7-(2-((4-((1R,5S)-3,8-diazabicyclo[3.2.1]octan-3-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide

### Step 1: tert-butyl (1R,5S)-3-(3-cyclopropyl-4-nitrophenyl)-3,8-diazabicyclo[3.2.1]octane-8-carboxylate

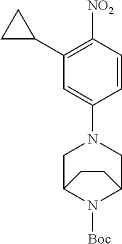 [SMILES: CC(C)(C)OC(=O)N1C2CCC1CN(c1ccc([N+](=O)[O-])c(C3CC3)c1)C2]

The title compound was prepared analogously to Example 305, step 1, where tert-butyl (1S,4S)-2,5-diazabicyclo[2.2.1]heptane-2-carboxylate was replaced with tert-butyl (1R,5S)-3,8-diazabicyclo[3.2.1]octane-8-carboxylate. The title compound was isolated in 71% yield. MS (ESI) m/z: 374.2 [M+H]⁺. ¹H NMR (400 MHz, DMSO-d₆) δ 7.89 (d, J=9.2 Hz, 1H), 6.80 (dd, J=9.2, 2.8 Hz, 1H), 6.50 (d, J=2.8 Hz, 1H), 4.28-4.19 (m, 2H), 3.71 (d, J=11.2 Hz, 2H), 2.97-2.90 (m, 2H), 2.49-2.44 (m, 1H), 1.90-1.78 (m, 2H), 1.73-1.63 (m, 2H), 1.41 (s, 9H), 0.98-0.92 (m, 2H), 0.78-0.73 (m, 2H).

### Step 2: tert-butyl (1R,5S)-3-(4-amino-3-cyclopropylphenyl)-3,8-diazabicyclo[3.2.1]octane-8-carboxylate

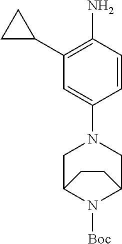 [SMILES: CC(C)(C)OC(=O)N1C2CCC1CN(c1ccc(N)c(C3CC3)c1)C2]

The title compound was prepared analogously to Example 173, step 4, where 1-(7-cyclopropyl-6-nitro-3,4-dihydro-1H-isoquinolin-2-yl)-2,2,2-trifluoro-ethanone was replaced tert-butyl (1R,5S)-3-(3-cyclopropyl-4-nitrophenyl)-3,8-diazabicyclo[3.2.1]octane-8-carboxylate. The title compound was isolated in 85% yield. MS (ESI) m/z: 344.3 [M+H]⁺. ¹H NMR (400 MHz, DMSO-d₆) δ 6.55-6.46 (m, 2H), 6.38 (d, J=2.0 Hz, 1H), 4.52-4.35 (m, 2H), 4.22-4.12 (m, 2H), 3.24-3.16 (m, 2H), 2.64-2.56 (m, 2H), 1.84-1.75 (m, 4H), 1.69-1.61 (m, 1H), 1.40 (s, 9H), 0.84-0.77 (m, 2H), 0.51-0.44 (m, 2H).

### Step 3: tert-butyl (1R,5S)-3-(4-((4-chloro-5-(trifluoromethyl)pyrimidin-2-yl)amino)-3-cyclopropylphenyl)-3,8-diazabicyclo[3.2.1]octane-8-carboxylate

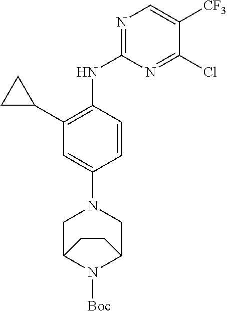 [SMILES: CC(C)(C)OC(=O)N1C2CCC1CN(c1ccc(Nc3ncc(C(F)(F)F)c(Cl)n3)c(C3CC3)c1)C2]

A mixture of tert-butyl 3-(4-amino-3-cyclopropyl-phenyl)-3,8-diazabicyclo[3.2.1]octane-8-carboxylate (27.1 mmol) and 2,4-dichloro-5-(trifluoromethyl)pyrimidine (135 mmol) was stirred at 70° C. for 12 hours. The reaction mixture was poured into water and extracted with ethyl acetate three times. The combined organic layers were washed with brine, dried over anhydrous sodium sulfate, filtered, and concentrated under reduced pressure to afford a residue that was purified by preparative-HPLC. The title compound was isolated in 20% yield. MS (ESI) m/z: 524.1 [M+H]⁺. ¹H NMR (400 MHz, DMSO-d₆) δ 9.95 (s, 1H), 8.74-8.49 (m, 1H), 7.05 (d, J=8.4 Hz, 1H), 6.68 (d, J=8.0 Hz, 1H), 6.38 (d, J=1.6 Hz, 1H), 4.26-4.14 (m, 2H), 3.53-3.45 (m, 2H), 2.81-2.70 (m, 2H), 1.89-1.73 (m, 5H), 1.41 (s, 9H), 0.81-0.73 (m, 2H), 0.65-0.57 (m, 2H).

### Step 4: tert-butyl (1R,5S)-3-(3-cyclopropyl-4-((5-(trifluoromethyl)-4-(trimethylstannyl)pyrimidin-2-yl)amino)phenyl)-3,8-diazabicyclo[3.2.1]octane-8-carboxylate

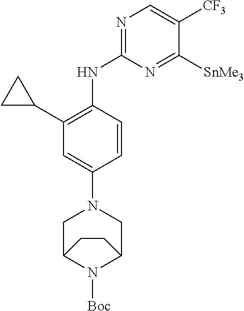

The title compound was prepared analogously to Example 4, step 1, where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with tert-butyl (1R,5S)-3-(4-((4-chloro-5-(trifluoromethyl)pyrimidin-2-yl)amino)-3-cyclopropylphenyl)-3,8-diazabicyclo[3.2.1]octane-8-carboxylate. The purification of this compound was carried out using neutral alumina. The title compound was isolated in 88% yield. MS (ESI) m/z: 654.1 [M+H]⁺.

### Step 5: tert-butyl (1R,5S)-3-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)-3,8-diazabicyclo[3.2.1]octane-8-carboxylate

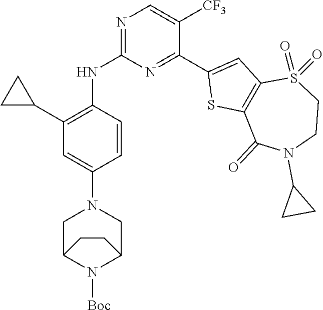 [SMILES: CC(C)(C)OC(=O)N1C2CCC1CN(c1ccc(Nc3ncc(C(F)(F)F)c(-c4cc5c(s4)C(=O)N(C4CC4)CCS5(=O)=O)n3)c(C3CC3)c1)C2]

The title compound was prepared analogously to Example 155, step 3, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one was replaced with tert-butyl (1R,5S)-3-(3-cyclopropyl-4-((5-(trifluoromethyl)-4-(trimethylstannyl)pyrimidin-2-yl)amino)phenyl)-3,8-diazabicyclo[3.2.1]octane-8-carboxylate. The title compound was isolated in 43% yield. MS (ESI) m/z: 745.3 [M+H]⁺.

### Step 6: 7-(2-((4-((1R,5S)-3,8-diazabicyclo[3.2.1]octan-3-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide

The title compound was prepared analogously to Example 305, step 6, where tert-butyl (1S,4S)-5-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)-2,5-diazabicyclo[2.2.1]heptane-2-carboxylate was replaced with tert-butyl (1R,5S)-3-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)-3,8-diazabicyclo[3.2.1]octane-8-carboxylate. The title compound was isolated in 48% yield. MS (ESI) m/z: 645.1 [M+H]⁺. ¹H NMR (400 MHz, DMSO-d₆) δ 9.75 (s, 1H), 8.87-8.61 (m, 1H), 7.78 (s, 1H), 7.25-7.05 (m, 1H), 6.73-6.61 (m, 1H), 6.36 (s, 1H), 3.92-3.82 (m, 4H), 3.70-3.62 (m, 3H), 3.54-3.50 (m, 2H), 2.91-2.83 (m, 1H), 2.82-2.75 (m, 2H), 1.94-1.88 (m, 1H), 1.82-1.69 (m, 4H), 0.84-0.74 (m, 6H), 0.64-0.57 (m, 2H).

### Example 308: (R)-4-cyclopropyl-7-(2-((2-cyclopropyl-4-(3-methylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

### Step 1: tert-butyl (R)-4-(3-cyclopropyl-4-nitrophenyl)-2-methylpiperazine-1-carboxylate

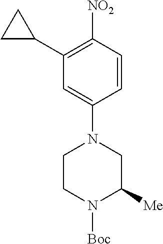 [SMILES: C[C@@H]1CN(c2ccc([N+](=O)[O-])c(C3CC3)c2)CCN1C(=O)OC(C)(C)C]

The title compound was prepared analogously to Example 305, step 1, where tert-butyl (1S,4S)-2,5-diazabicyclo[2.2.1]heptane-2-carboxylate was replaced with tert-butyl (R)-2-methylpiperazine-1-carboxylate. The title compound was isolated in 79% yield. MS (ESI) m/z: 362.1 [M+H]⁺.

### Step 2: tert-butyl (R)-4-(4-amino-3-cyclopropylphenyl)-2-methylpiperazine-1-carboxylate

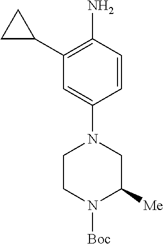 [SMILES: C[C@@H]1CN(c2ccc(N)c(C3CC3)c2)CCN1C(=O)OC(C)(C)C]

The title compound was prepared analogously to Example 173, step 4, where 1-(7-cyclopropyl-6-nitro-3,4-dihydro-1H-isoquinolin-2-yl)-2,2,2-trifluoro-ethanone was replaced with tert-butyl (R)-4-(3-cyclopropyl-4-nitrophenyl)-2-methylpiperazine-1-carboxylate. The title compound was isolated in 78% yield. MS (ESI) m/z: 332.2 [M+H]⁺. ¹H NMR (400 MHz, DMSO-d₆) δ=6.56 (d, J=2.4 Hz, 1H), 6.55 (s, 1H), 6.44 (d, J=2.4 Hz, 1H), 4.59-4.51 (m, 2H), 4.18-4.10 (m, 1H), 3.75 (d, J=13.2 Hz, 1H), 3.24 (d, J=11.2 Hz, 1H), 3.14 (m, 1H), 3.10-3.04 (m, 1H), 2.59-2.55 (m, 1H), 2.42-2.35 (m, 1H), 1.69-1.62 (m, 1H), 1.42 (s, 9H), 1.22 (d, J=6.4 Hz, 3H), 0.84-0.81 (m, 2H), 0.50-0.46 (m, 2H).

### Step 3: tert-butyl (R)-4-(4-((4-chloro-5-(trifluoromethyl)pyrimidin-2-yl)amino)-3-cyclopropylphenyl)-2-methylpiperazine-1-carboxylate

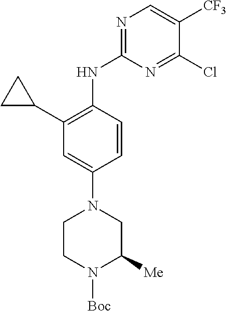 [SMILES: C[C@@H]1CN(c2ccc(Nc3ncc(C(F)(F)F)c(Cl)n3)c(C3CC3)c2)CCN1C(=O)OC(C)(C)C]

The title compound was prepared analogously to Example 305, step 3, where tert-butyl (1S,4S)-5-(4-amino-3-cyclopropylphenyl)-2,5-diazabicyclo[2.2.1]heptane-2-carboxylate was replaced with tert-butyl (R)-4-(4-amino-3-cyclopropylphenyl)-2-methylpiperazine-1-carboxylate. The title compound was isolated in 20% yield. MS (ESI) m/z: 512.2 [M+H]⁺. ¹H NMR (400 MHz, DMSO-d₆) δ=9.97 (s, 1H), 8.80-8.53 (m, 1H), 7.09 (d, J=8.8 Hz, 1H), 6.87-6.67 (m, 1H), 6.45 (d, J=2.4 Hz, 1H), 4.24-4.15 (m, 1H), 3.79 (d, J=13.2 Hz, 1H), 3.57 (d, J=11.2 Hz, 1H), 3.46 (d, J=11.2 Hz, 2H), 3.23-3.10 (m, 1H), 2.82-2.78 (m, 1H), 2.60 (m, 1H), 1.92-1.80 (m, 1H), 1.42 (s, 9H), 1.21-1.19 (m, 1H), 1.22-1.18 (m, 1H), 0.86-0.75 (m, 2H), 0.61 (d, J=3.6 Hz, 2H).

### Step 4: tert-butyl (R)-4-(3-cyclopropyl-4-((5-(trifluoromethyl)-4-(trimethylstannyl)pyrimidin-2-yl)amino)phenyl)-2-methylpiperazine-1-carboxylate

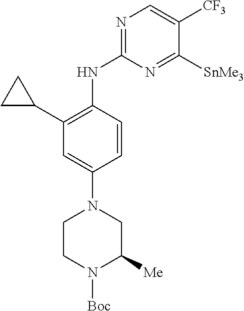

The title compound was prepared analogously to Example 4, step 1, where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with tert-butyl (R)-4-(4-((4-chloro-5-(trifluoromethyl)pyrimidin-2-yl)amino)-3-cyclopropylphenyl)-2-methylpiperazine-1-carboxylate. The purification of this compound was carried out using neutral alumina. The title compound was isolated in 39% yield. MS (ESI) m/z: 642.1 [M+H]⁺. ¹H NMR (400 MHz, DMSO-d₆) δ=9.19 (s, 1H), 8.51-8.33 (m, 1H), 7.21 (d, J=4.0 Hz, 1H), 6.74-6.70 (m, 1H), 6.43 (d, J=2.4 Hz, 1H), 4.20 (d, J=3.6 Hz, 1H), 3.78 (d, J=13.2 Hz, 1H), 3.53 (d, J=12.0 Hz, 1H), 3.42 (d, J=12.0 Hz, 1H), 3.18-3.11 (m, 1H), 2.78-2.74 (m, 1H), 2.59-2.53 (m, 1H), 1.93-1.86 (m, 1H), 1.42 (s, 9H), 1.19 (d, J=1.2 Hz, 3H), 0.81-0.77 (m, 2H), 0.61-0.58 (m, 2H), 0.28 (s, 9H).

### Step 5: tert-butyl (R)-4-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)-2-methylpiperazine-1-carboxylate

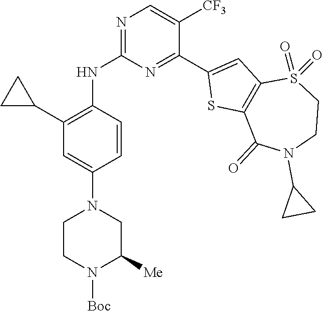 [SMILES: C[C@@H]1CN(c2ccc(Nc3ncc(C(F)(F)F)c(-c4cc5c(s4)C(=O)N(C4CC4)CCS5(=O)=O)n3)c(C3CC3)c2)CCN1C(=O)OC(C)(C)C]

The title compound was prepared analogously to Example 155, step 3, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one was replaced with tert-butyl (R)-4-(3-cyclopropyl-4-((5-(trifluoromethyl)-4-(trimethylstannyl)pyrimidin-2-yl)amino)phenyl)-2-methylpiperazine-1-carboxylate. The title compound was isolated in 66% yield. MS (ESI) m/z: 733.2 [M+H]⁺. ¹H NMR (400 MHz, DMSO-d₆) δ 9.77 (s, 1H), 8.80-8.71 (m, 1H), 7.78 (s, 1H), 7.19-7.14 (m, 1H), 6.76 (d, J=8.4 Hz, 1H), 6.46 (s, 1H), 4.22-4.20 (m, 1H), 3.86-3.81 (m, 4H), 3.79 (d, J=13.2 Hz, 1H), 3.55-3.45 (m, 1H), 3.20-3.16 (m, 1H), 2.88-2.81 (m, 1H), 2.80-2.75 (m, 1H), 2.62-2.59 (m, 1H), 1.99-1.91 (m, 2H), 1.42 (s, 9H), 1.21-1.19 (m, 3H), 0.87-0.79 (m, 6H), 0.64-0.62 (m, 4H).

### Step 6: (R)-4-cyclopropyl-7-(2-((2-cyclopropyl-4-(3-methylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 305, step 6, where tert-butyl (1S,4S)-5-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)-2,5-diazabicyclo[2.2.1]heptane-2-carboxylate was replaced with tert-butyl (R)-4-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)-2-methylpiperazine-1-carboxylate. The title compound was isolated in 24% yield. MS (ESI) m/z: 633.1 [M+H]⁺. ¹H NMR (400 MHz, CDCl₃) δ=8.70 (s, 1H), 8.06 (s, 1H), 8.00-7.85 (m, 1H), 7.77-7.60 (m, 1H), 6.90 (d, J=8.4 Hz, 1H), 6.75 (s, 1H), 5.45-5.26 (m, 1H), 3.95 (t, J=5.6 Hz, 2H), 3.65 (t, J=6.0 Hz, 2H), 3.60-3.51 (m, 2H), 3.28-3.09 (m, 2H), 2.95-2.86 (m, 2H), 2.65-2.53 (m, 1H), 2.23 (t, J=7.6 Hz, 1H), 2.05-2.00 (m, 1H), 1.32 (s, 3H), 1.06-1.02 (m, 2H), 0.98-0.95 (m, 2H), 0.89-0.85 (m, 2H), 0.71 (d, J=5.6 Hz, 2H).

### Example 309: (R)-4-cyclopropyl-7-(2-((2-cyclopropyl-4-(3,4-dimethylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (R)-4-cyclopropyl-7-(2-((2-cyclopropyl-4-(3-methylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide. The title compound was isolated in 35% yield. MS (ESI) m/z: 647.2 [M+H]⁺. ¹H NMR (400 MHz, CDCl₃) δ=8.69 (s, 1H), 8.05 (s, 1H), 7.98-7.80 (m, 1H), 7.78-7.60 (m, 1H), 6.89 (d, J=8.0 Hz, 1H), 6.75 (s, 1H), 3.94 (t, J=6.0 Hz, 2H), 3.64 (t, J=6.0 Hz, 2H), 3.52 (d, J=11.2 Hz, 1H), 3.45 (d, J=11.6 Hz, 1H), 2.95 (d, J=3.6 Hz, 1H), 2.94-2.90 (m, 2H), 2.60-2.53 (m, 1H), 2.51-2.42 (m, 1H), 2.37 (s, 3H), 2.34-2.27 (m, 1H), 1.93-1.85 (m, 1H), 1.18 (d, J=6.0 Hz, 3H), 1.06-1.00 (m, 2H), 0.95 (d, J=4.4 Hz, 4H), 0.75-0.68 (m, 2H).

### Example 310: 7-(2-((4-((1S,4S)-2,5-diazabicyclo[2.2.1]heptan-2-yl)-2-cyclopropylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

### Step 1: N-(4-((1S,4S)-2,5-diazabicyclo[2.2.1]heptan-2-yl)-2-cyclopropylphenyl)-4-chloro-5-(trifluoromethyl)pyrimidin-2-amine

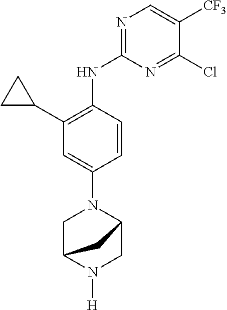 [SMILES: FC(F)(F)c1cnc(Nc2ccc(N3C[C@@H]4C[C@H]3CN4)cc2C2CC2)nc1Cl]

The title compound was prepared analogously to Example 305, step 6, where tert-butyl (1S,4S)-5-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)-2,5-diazabicyclo[2.2.1]heptane-2-carboxylate was replaced with tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate. The title compound was isolated in 97% yield. MS (ESI) m/z: 410.2 [M+H]⁺. ¹H NMR (400 MHz, methanol-d₄) δ 7.25 (d, J=8.4 Hz, 1H), 6.55 (dd, J=8.4 Hz, 2.8, 1H), 6.31 (d, J=2.4 Hz, 1H), 4.67 (s, 2H), 3.74 (dd, J=10.8, 2.4 Hz, 1H), 3.41-3.35 (m, 2H), 2.30 (d, J=10.8 Hz, 1H), 2.13-1.99 (m, 2H), 1.94-1.87 (m, 1H), 0.88 (d, J=8.4 Hz, 2H), 0.68-0.59 (m, 2H).

### Step 2: 1-((1S,4S)-5-(4-((4-chloro-5-(trifluoromethyl)pyrimidin-2-yl)amino)-3-cyclopropylphenyl)-2,5-diazabicyclo[2.2.1]heptan-2-yl)-2,2,2-trifluoroethan-1-one

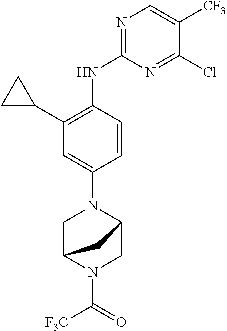 [SMILES: O=C(N1C[C@@H]2C[C@H]1CN2c1ccc(Nc2ncc(C(F)(F)F)c(Cl)n2)c(C2CC2)c1)C(F)(F)F]

The title compound was prepared analogously to Example 9, step 2, where 7-ethyl-6-nitro-1,2,3,4-tetrahydroisoquinoline was replaced with N-(4-((1S,4S)-2,5-diazabicyclo[2.2.1]heptan-2-yl)-2-cyclopropylphenyl)-4-chloro-5-(trifluoromethyl)pyrimidin-2-amine. The title compound was isolated in 60% yield. MS (ESI) m/z: 506.3 [M+H]*. ¹H NMR (400 MHz, methanol-d₄) δ 8.72-8.20 (m, 1H), 7.20 (d, J=8.4 Hz, 1H), 6.63-6.45 (m, 1H), 6.40-6.18 (m, 1H), 5.06-4.90 (m, 1H), 4.64 (d, J=14.8 Hz, 1H), 3.84-3.62 (m, 2H), 3.59 (s, 1H), 3.15 (d, J=9.6 Hz, 1H), 2.26-2.02 (m, 2H), 1.94-1.80 (m, 1H), 0.89-0.84 (m, 2H), 0.73-0.58 (m, 2H).

### Step 3: 1-((1S,4S)-5-(3-cyclopropyl-4-((5-(trifluoromethyl)-4-(trimethylstannyl)pyrimidin-2-yl)amino)phenyl)-2,5-diazabicyclo[2.2.1]heptan-2-yl)-2,2,2-trifluoroethan-1-one

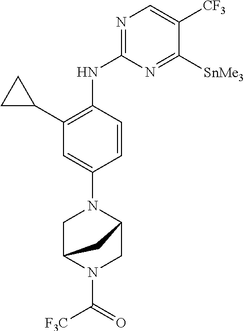

The title compound was prepared analogously to Example 4, step 1, where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced 1-((1S,4S)-5-(4-((4-chloro-5-(trifluoromethyl)pyrimidin-2-yl)amino)-3-cyclopropylphenyl)-2,5-diazabicyclo[2.2.1]heptan-2-yl)-2,2,2-trifluoroethan-1-one. The purification of this compound was carried out using neutral alumina. The title compound was isolated in 93% yield. MS (ESI) m/z: 634.1 [M+H]⁺. ¹H NMR (400 MHz, methanol-d₄) δ 8.52-8.28 (m, 1H), 7.47-7.10 (m, 1H), 6.58-6.40 (m, 1H), 6.27 (dd, J=6.0, 2.8 Hz, 1H), 4.98 (s, 1H), 4.62 (d, J=14.4 Hz, 1H), 3.81-3.60 (m, 2H), 3.58 (s, 1H), 3.13 (dd, J=8.8, 4.0 Hz, 1H), 2.28-2.01 (m, 2H), 1.93-1.84 (m, 1H), 0.88-0.84 (m, 4H), 0.64-0.61 (m, 2H), 0.45-0.16 (m, 7H).

### Step 4: 7-(2-((2-cyclopropyl-4-((1S,4S)-5-(2,2,2-trifluoroacetyl)-2,5-diazabicyclo[2.2.1]heptan-2-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

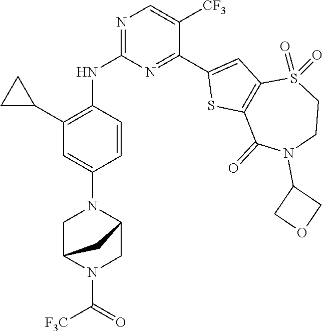 [SMILES: O=C1c2sc(-c3nc(Nc4ccc(N5C[C@@H]6C[C@H]5CN6C(=O)C(F)(F)F)cc4C4CC4)ncc3C(F)(F)F)cc2S(=O)(=O)CCN1C1COC1]

The title compound was prepared analogously to Example 155, step 3, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-((1S,4S)-5-(3-cyclopropyl-4-((5-(trifluoromethyl)-4-(trimethylstannyl)pyrimidin-2-yl)amino)phenyl)-2,5-diazabicyclo[2.2.1]heptan-2-yl)-2,2,2-trifluoroethan-1-one and 7-iodo-4-(oxetan-3-yl)-3,4-dihydrothieno[2,3-f][1,4]thiazepin-5(2H)-one 1,1-dioxide. The title compound was isolated in 72% yield. MS (ESI) m/z: 743.4 [M+H]⁺.

### Step 5: 7-(2-((4-((1S,4S)-2,5-diazabicyclo[2.2.1]heptan-2-yl)-2-cyclopropylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 59, step 6, where 5-(2-((7-chloro-2-(2,2,2-trifluoroacetyl)-1,2,3,4-tetrahydroisoquinolin-6-yl)amino)-5-(trifluoromethyl)pyrimidin-4-yl)-3-(methylsulfonyl)thiophene-2-carboxamide was replaced with 7-(2-((2-cyclopropyl-4-((1S,4S)-5-(2,2,2-trifluoroacetyl)-2,5-diazabicyclo[2.2.1]heptan-2-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 97% yield. MS (ESI) m/z: 647.4 [M+H]⁺. ¹H NMR (400 MHz, methanol-d₄) δ 8.75-8.52 (m, 1H), 7.99 (s, 1H), 7.35-7.23 (m, 1H), 6.65-6.51 (m, 1H), 6.32 (s, 1H), 5.38-5.14 (m, 1H), 4.63 (s, 3H), 4.37 (s, 2H), 4.10-4.00 (m, 2H), 3.89 (d, J=4.8 Hz, 2H), 3.73 (d, J=8.8 Hz, 2H), 3.35 (s, 2H), 3.27 (s, 1H), 2.26 (d, J=10.1 Hz, 1H), 2.08-1.95 (m, 2H), 0.89 (m, 2H), 0.69-0.61 (m, 2H).

### Example 311: 7-(2-((7-cyclopropyl-1,2,3,4-tetrahydroisoquinolin-6-yl)amino)-5-(trifluoromethyl)pyrimidin-4-yl)-3,4-dihydro-2H-thieno[3,2-f][1,5,2]dithiazepine 1,1,5,5-tetraoxide

The title compound was prepared analogously to Example 155, steps 3-4, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with 1-(7-cyclopropyl-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-iodo-3,4-dihydro-2H-thieno[3,2-f][1,5,2]dithiazepine 1,1,5,5-tetraoxide in step 3. The title compound was isolated. MS (ESI) m/z: 586.2 [M+H]⁺. ¹H NMR (400 MHz, DMSO-d₆) δ 9.80 (s, 1H), 8.79 (s, 1H), 7.85 (s, 1H), 7.38-7.06 (m, 1H), 6.72 (s, 1H), 3.93 (s, 2H), 3.75-3.70 (m, 2H), 3.56 (d, J=5.2 Hz, 2H), 3.08-3.05 (m, 2H), 2.76 (s, 2H), 1.96-1.92 (m, 1H), 0.85-0.80 (m, 2H), 0.59-0.52 (m, 2H).

### Example 312: 7-(2-((7-cyclopropyl-2-(2-(trifluoromethoxy)ethyl)-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 227, where 7-(2-((7-chloro-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide and 1-bromo-2-fluoroethane were replaced with 7-(2-((7-cyclopropyl-1,2,3,4-tetrahydroisoquinolin-6-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide and 1-bromo-2-(trifluoromethoxy)ethane. The title compound was isolated in 7% yield. MS (ESI) m/z: 718.4 [M+H]⁺. ¹H NMR (400 MHz, CDCl₃) δ 8.75 (s, 1H), 8.12 (s, 1H), 7.91 (s, 1H), 7.85 (s, 1H), 6.86 (s, 1H), 5.58-5.51 (m, 1H), 5.04 (t, J=7.4 Hz, 2H), 4.74 (t, J=6.8 Hz, 2H), 4.17 (t, J=5.8 Hz, 2H), 4.14 (t, J=5.6 Hz, 2H), 3.79 (t, J=5.8 Hz, 2H), 3.69 (s, 2H), 3.03-2.91 (m, 2H), 2.87 (td, J=5.8, 14.2 Hz, 4H), 1.88-1.76 (m, 1H), 1.06-1.01 (m, 2H), 0.70-0.64 (m, 2H).

### Example 313: 4-cyclopropyl-7-(2-((2-cyclopropyl-4-((1R,5S)-8-methyl-3,8-diazabicyclo[3.2.1]octan-3-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306 where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((4-((1R,5S)-3,8-diazabicyclo[3.2.1]octan-3-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide. The title compound was isolated in 80% yield. MS (ESI) m/z: 659.2 [M+H]⁺. ¹H NMR (400 MHz, DMSO-d₆) δ 9.72 (s, 1H), 8.88-8.59 (m, 1H), 7.86-7.68 (m, 1H), 7.22-7.03 (m, 1H), 6.70-6.56 (m, 1H), 6.40-6.28 (m, 1H), 3.96-3.79 (m, 4H), 3.21-3.17 (m, 2H), 2.96-2.74 (m, 4H), 2.22 (s, 3H), 2.06-1.82 (m, 4H), 1.68-1.57 (m, 2H), 0.86-0.76 (m, 6H), 0.65-0.55 (m, 2H).

### Example 314: 4-cyclopropyl-7-(2-((7-cyclopropyl-2-(2-(trifluoromethoxy)ethyl)-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 227, where 7-(2-((7-chloro-1,2,3,4-tetrahydroisoquinolin-6-yl)amino)-5-(trifluoromethyl)pyrimidin-4-yl)-4-cyclopropyl-3,4-dihydrothieno[2,3-f][1,4]thiazepin-5(2H)-one 1,1-dioxide and 1-bromo-2-fluoroethane were replaced with 4-cyclopropyl-7-(2-((7-cyclopropyl-1,2,3,4-tetrahydroisoquinolin-6-yl)amino)-5-(trifluoromethyl)pyrimidin-4-yl)-3,4-dihydrothieno[2,3-f][1,4]thiazepin-5(2H)-one 1,1-dioxide and 1-bromo-2-(trifluoromethoxy)ethane. The title compound was isolated in 7% yield. MS (ESI) m/z: 702.4 [M+H]⁺. ¹H NMR (400 MHz, CDCl₃) δ 8.74 (s, 1H), 8.08 (s, 1H), 7.99-7.83 (m, 2H), 6.86 (s, 1H), 4.17 (t, J=6.0 Hz, 2H), 3.96 (t, J=5.8 Hz, 2H), 3.77-3.70 (m, 2H), 3.69 (s, 2H), 3.65 (t, J=5.6 Hz, 2H), 3.03-2.95 (m, 2H), 2.94-2.91 (m, 1H), 2.88 (d, J=5.4 Hz, 2H), 1.89-1.78 (m, 1H), 1.06-1.01 (m, 2H), 0.99-0.93 (m, 4H), 0.71-0.65 (m, 2H).

### Example 315: (R)-7-(2-((2-cyclopropyl-4-(3-methylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 310, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (R)-4-(4-((4-chloro-5-(trifluoromethyl)pyrimidin-2-yl)amino)-3-cyclopropylphenyl)-2-methylpiperazine-1-carboxylate in step 1. The title compound was isolated. MS (ESI) m/z: 649.1 [M+H]⁺. ¹H NMR (400 MHz, CDCl₃) δ=8.71 (s, 1H), 8.09 (s, 1H), 7.95-7.82 (m, 1H), 7.78-7.59 (m, 1H), 6.89-6.87 (m, 1H), 6.75 (s, 1H), 5.63-5.49 (m, 1H), 5.03 (t, J=7.6 Hz, 2H), 4.74 (t, J=6.8 Hz, 2H), 4.17-4.07 (m, 2H), 3.78 (t, J=5.6 Hz, 2H), 3.51 (d, J=10.4 Hz, 2H), 3.20-3.11 (m, 1H), 3.10-2.96 (m, 2H), 2.80-2.66 (m, 1H), 2.45-2.32 (m, 1H), 2.02-1.78 (m, 1H), 1.16 (d, J=6.4 Hz, 3H), 1.05-0.99 (m, 2H), 0.92-0.86 (m, 1H), 0.74-0.69 (m, 2H).

### Example 316: (R)-7-(2-((2-cyclopropyl-4-(3,4-dimethylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (R)-7-(2-((2-cyclopropyl-4-(3-methylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 89% yield. MS (ESI) m/z: 663.2 [M+H]⁺. ¹H NMR (400 MHz, CDCl₃) δ=8.70 (s, 1H), 8.09 (s, 1H), 7.92-7.80 (m, 1H), 7.77-7.58 (m, 1H), 6.92-6.84 (m, 1H), 6.74 (d, J=2.4 Hz, 1H), 5.62-5.49 (m, 1H), 5.03 (t, J=7.6 Hz, 2H), 4.74 (t, J=6.8 Hz, 2H), 4.12 (t, J=5.6 Hz, 2H), 3.78 (t, J=5.6 Hz, 2H), 3.57-3.39 (m, 2H), 3.00-2.87 (m, 2H), 2.55 (t, J=10.4 Hz, 1H), 2.48-2.41 (m, 1H), 2.37 (s, 3H), 2.30-2.20 (m, 1H), 1.94-1.82 (m, 1H), 1.17 (d, J=6.4 Hz, 3H), 1.07-1.00 (m, 2H), 0.74-0.67 (m, 2H).

### Example 317: 7-(2-((2-cyclopropyl-4-((1S,4S)-5-methyl-2,5-diazabicyclo[2.2.1]heptan-2-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((4-((1S,4S)-2,5-diazabicyclo[2.2.1]heptan-2-yl)-2-cyclopropylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 19% yield. MS (ESI) m/z: 661.2 [M+H]⁺. ¹H NMR (400 MHz, methanol-d₄) δ 8.78-8.58 (m, 1H), 8.01 (s, 1H), 7.37-7.17 (m, 1H), 6.61-6.52 (m, 1H), 6.31 (s, 1H), 5.35-5.26 (m, 1H), 4.92 (s, 2H), 4.84-4.83 (m, 2H), 4.58-4.43 (m, 2H), 4.13-4.02 (m, 2H), 4.00-3.94 (m, 1H), 3.92-3.86 (m, 2H), 3.69-3.59 (m, 1H), 3.38 (s, 1H), 3.08 (d, J=10.4 Hz, 1H), 2.68 (s, 3H), 2.22-2.17 (m, 1H), 2.05-1.94 (m, 2H), 0.89 (m, 2H), 0.66 (m, 2H).

### Example 318: 7-(2-((4-((1R,5S)-3,8-diazabicyclo[3.2.1]octan-3-yl)-2-cyclopropylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 310, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (1R,5S)-3-(4-((4-chloro-5-(trifluoromethyl)pyrimidin-2-yl)amino)-3-cyclopropylphenyl)-3,8-diazabicyclo[3.2.1]octane-8-carboxylate. The title compound was isolated. MS (ESI) m/z: 661.3 [M+H]⁺. ¹H NMR (400 MHz, DMSO-d₆) δ 9.74 (s, 1H), 8.85-8.62 (m, 1H), 7.89-7.71 (m, 1H), 7.22-7.04 (m, 1H), 6.67-6.60 (m, 1H), 6.33 (s, 1H), 5.28-5.17 (m, 1H), 4.79-4.64 (m, 4H), 4.10-3.90 (m, 4H), 3.59-3.53 (m, 2H), 3.42-3.39 (m, 2H), 2.78-2.70 (m, 2H), 2.02-1.97 (m, 1H), 1.74-1.64 (m, 4H), 0.87-0.83 (m, 1H), 0.81-0.76 (m, 2H), 0.63-0.57 (m, 2H).

### Example 319: 7-(2-((4-((1S,4S)-2,5-diazabicyclo[2.2.1]heptan-2-yl)-2-ethylphenyl)amino)-5-(trifluoromethyl)pyrimidin-4-yl)-4-cyclopropyl-3,4-dihydrothieno[2,3-f][1,4]thiazepin-5(2H)-one 1,1-dioxide

### Step 1: tert-butyl (1S,4S)-5-(3-bromo-4-nitrophenyl)-2,5-diazabicyclo[2.2.1]heptane-2-carboxylate

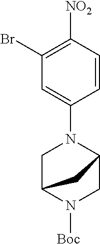 [SMILES: CC(C)(C)OC(=O)N1C[C@@H]2C[C@H]1CN2c1ccc([N+](=O)[O-])c(Br)c1]

The title compound was prepared analogously to Example 305, step 1, where 2-cyclopropyl-4-fluoro-1-nitrobenzene, cesium carbonate and DMF were replaced with 2-bromo-4-fluoro-1-nitrobenzene, potassium carbonate and NMP. The title compound was isolated in 99% yield. MS (ESI) m/z: 398.1 [M+H]⁺.

### Step 2: tert-butyl (1S,4S)-5-(4-nitro-3-vinylphenyl)-2,5-diazabicyclo[2.2.1]heptane-2-carboxylate

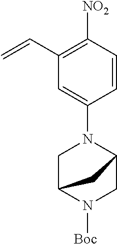 [SMILES: C=Cc1cc(N2C[C@@H]3C[C@H]2CN3C(=O)OC(C)(C)C)ccc1[N+](=O)[O-]]

The title compound was prepared analogously to Example 212, step 3, where 1-(6-bromo-7-nitro-3,4-dihydro-1H-isoquinolin-2-yl)-2,2,2-trifluoro-ethanone was replaced with tert-butyl (1S,4S)-5-(3-bromo-4-nitrophenyl)-2,5-diazabicyclo[2.2.1]heptane-2-carboxylate. The title compound was isolated in 58% yield. MS (ESI) m/z: 346.2 [M+H]⁺.

### Step 3: tert-butyl (1S,4S)-5-(4-amino-3-ethylphenyl)-2,5-diazabicyclo[2.2.1]heptane-2-carboxylate

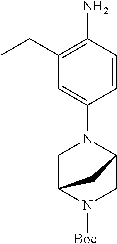 [SMILES: CCc1cc(N2C[C@@H]3C[C@H]2CN3C(=O)OC(C)(C)C)ccc1N]

The title compound was prepared analogously to Example 212, step 4, where 2,2,2-trifluoro-1-(7-nitro-6-vinyl-3,4-dihydroisoquinolin-2(1H)-yl)ethan-1-one was replaced with tert-butyl (1S,4S)-5-(4-nitro-3-vinylphenyl)-2,5-diazabicyclo[2.2.1]heptane-2-carboxylate. The title compound was isolated in 93% yield. MS (ESI) m/z: 318.2 [M+H]⁺.

### Step 4: tert-butyl (1S,4S)-5-(4-((4-chloro-5-(trifluoromethyl)pyrimidin-2-yl)amino)-3-ethylphenyl)-2,5-diazabicyclo[2.2.1]heptane-2-carboxylate

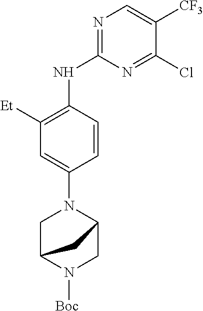 [SMILES: CCc1cc(N2C[C@@H]3C[C@H]2CN3C(=O)OC(C)(C)C)ccc1Nc1ncc(C(F)(F)F)c(Cl)n1]

The title compound was prepared analogously to Example 305, step 3, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (1S,4S)-5-(4-amino-3-ethylphenyl)-2,5-diazabicyclo[2.2.1]heptane-2-carboxylate. The title compound was isolated in 99% yield. MS (ESI) m/z: 498.3 [M+H]⁺. ¹H NMR (400 MHz, DMSO-d₆) δ 9.82 (s, 1H), 8.76-8.48 (m, 1H), 7.01 (d, J=8.0 Hz, 1H), 6.56-6.42 (m, 2H), 4.56-4.36 (m, 2H), 3.60-3.49 (m, 1H), 3.29-3.15 (m, 2H), 2.98 (d, J=0.8 Hz, 1H), 2.46-2.43 (m, 2H), 1.95-1.86 (m, 2H), 1.37 (d, J=17.6 Hz, 9H), 1.07 (t, J=7.2 Hz, 3H).

### Step 5: tert-butyl (1S,4S)-5-(3-ethyl-4-((5-(trifluoromethyl)-4-(trimethylstannyl)pyrimidin-2-yl)amino)phenyl)-2,5-diazabicyclo[2.2.1]heptane-2-carboxylate

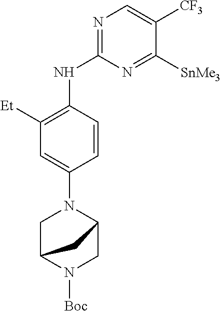

The title compound was prepared analogously to Example 305, step 4, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (1S,4S)-5-(4-((4-chloro-5-(trifluoromethyl)pyrimidin-2-yl)amino)-3-ethylphenyl)-2,5-diazabicyclo[2.2.1]heptane-2-carboxylate. The title compound was isolated in 37% yield. MS (ESI) m/z: 626.4 [M+H]⁺.

### Step 6: tert-butyl (1S,4S)-5-(4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)-3-ethylphenyl)-2,5-diazabicyclo[2.2.1]heptane-2-carboxylate

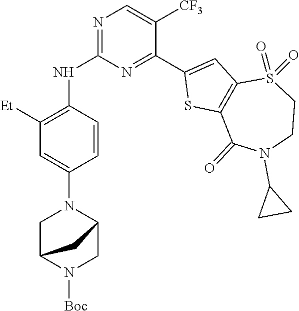 [SMILES: CCc1cc(N2C[C@@H]3C[C@H]2CN3C(=O)OC(C)(C)C)ccc1Nc1ncc(C(F)(F)F)c(-c2cc3c(s2)C(=O)N(C2CC2)CCS3(=O)=O)n1]

The title compound was prepared analogously to Example 305, step 5, where tert-butyl (1S,4S)-5-(3-cyclopropyl-4-((5-(trifluoromethyl)-4-(trimethylstannyl)pyrimidin-2-yl)amino)phenyl)-2,5-diazabicyclo[2.2.1]heptane-2-carboxylate was replaced with tert-butyl (1S,4S)-5-(3-ethyl-4-((5-(trifluoromethyl)-4-(trimethylstannyl)pyrimidin-2-yl)amino)phenyl)-2,5-diazabicyclo[2.2.1]heptane-2-carboxylate. The title compound was isolated in 79% yield. MS (ESI) m/z: 719.4 [M+H]⁺

### Step 7: 7-(2-((4-((1S,4S)-2,5-diazabicyclo[2.2.1]heptan-2-yl)-2-ethylphenyl)amino)-5-(trifluoromethyl)pyrimidin-4-yl)-4-cyclopropyl-3,4-dihydrothieno[2,3-f][1,4]thiazepin-5(2H)-one 1,1-dioxide

The title compound was prepared analogously to Example 305, step 6, where tert-butyl (1S,4S)-5-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)-2,5-diazabicyclo[2.2.1]heptane-2-carboxylate was replaced with tert-butyl (1S,4S)-5-(4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)-3-ethylphenyl)-2,5-diazabicyclo[2.2.1]heptane-2-carboxylate. The title compound was isolated in 70% yield. MS (ESI) m/z: 619.3 [M+H]⁺. ¹H NMR (400 MHz, CDCl₃) δ 8.65 (s, 1H), 7.99 (s, 1H), 7.34-7.28 (m, 1H), 7.04 (s, 1H), 6.50 (s, 2H), 4.45 (s, 1H), 4.40-4.21 (m, 1H), 3.93 (s, 2H), 3.82-3.73 (m, 1H), 3.63 (s, 2H), 3.43-3.18 (m, 4H), 2.91 (dd, J=4.0, 8.0 Hz, 1H), 2.58 (q, J=7.2 Hz, 2H), 2.26-2.05 (m, 2H), 1.19 (t, J=7.2 Hz, 3H), 0.97 (d, J=6.8 Hz, 2H), 0.88 (d, J=7.2 Hz, 2H).

### Example 320: 7-(2-((2-cyclopropyl-4-((1R,5S)-8-methyl-3,8-diazabicyclo[3.2.1]octan-3-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((4-((1R,5S)-3,8-diazabicyclo[3.2.1]octan-3-yl)-2-cyclopropylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 23% yield. MS (ESI) m/z: 675.1 [M+H]⁺. ¹H NMR (400 MHz, DMSO-d₆) δ 9.73 (s, 1H), 8.87-8.61 (m, 1H), 7.88-7.73 (m, 1H), 7.20-7.00 (m, 1H), 6.67-6.59 (m, 1H), 6.35 (s, 1H), 5.30-5.15 (m, 1H), 4.79-4.62 (m, 4H), 4.14-3.89 (m, 4H), 3.50-3.43 (m, 1H), 3.17-3.14 (m, 2H), 2.88-2.82 (m, 2H), 2.40-2.35 (m, 2H), 2.33 (s, 3H), 2.02-1.95 (m, 2H), 1.74-1.63 (m, 2H), 0.88-0.82 (m, 1H), 0.81-0.76 (m, 2H), 0.64-0.57 (m, 2H).

### Example 321: 4-cyclopropyl-7-(2-((2-ethyl-4-((1S,4S)-5-methyl-2,5-diazabicyclo[2.2.1]heptan-2-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((4-((1S,4S)-2,5-diazabicyclo[2.2.1]heptan-2-yl)-2-ethylphenyl)amino)-5-(trifluoromethyl)pyrimidin-4-yl)-4-cyclopropyl-3,4-dihydrothieno[2,3-f][1,4]thiazepin-5(2H)-one 1,1-dioxide. The title compound was isolated in 55% yield. MS (ESI) m/z: 633.4 [M+H]⁺. ¹H NMR (400 MHz, CDCl₃) δ 8.65 (s, 1H), 8.10-7.91 (m, 1H), 7.34-7.29 (m, 1H), 7.05-6.92 (m, 1H), 6.51 (br s, 2H), 4.55-4.29 (m, 1H), 3.99-3.90 (m, 2H), 3.63 (br d, J=6.4 Hz, 4H), 3.18-3.07 (m, 1H), 2.95-2.90 (m, 1H), 2.85-2.69 (m, 2H), 2.63-2.57 (m, 2H), 2.26-2.14 (m, 2H), 2.07 (s, 3H), 1.27 (s, 3H), 0.93-0.88 (m, 4H).

### Example 322: 7-(2-((4-((1R,4R)-2,5-diazabicyclo[2.2.1]heptan-2-yl)-2-ethylphenyl)amino)-5-(trifluoromethyl)pyrimidin-4-yl)-4-cyclopropyl-3,4-dihydrothieno[2,3-f][1,4]thiazepin-5(2H)-one 1,1-dioxide

The title compound was prepared analogously to Example 319, where tert-butyl (1S,4S)-2,5-diazabicyclo[2.2.1]heptane-2-carboxylate was replaced with tert-butyl (1R,4R)-2,5-diazabicyclo[2.2.1]heptane-2-carboxylate. The title compound was isolated. MS (ESI) m/z: 619.3 [M+H]⁺. ¹H NMR (400 MHz, CDCl₃-d) δ, 8.65 (s, 1H), 7.99 (br s, 1H), 7.29 (br d, J=9.6 Hz, 1H), 7.09-7.00 (m, 1H), 6.50 (br s, 2H), 4.60-4.14 (m, 2H), 3.93 (m, 2H), 3.64 (m, 2H), 3.59-3.34 (m, 2H), 3.34-2.97 (m, 2H), 2.96-2.89 (m, 1H), 2.60-2.55 (m, 2H), 2.24-2.15 (m, 2H), 1.19 (br t, J=7.2 Hz, 3H), 0.98 (m, 2H), 0.88 (m, 2H).

### Example 323: 4-cyclopropyl-7-(2-((2-ethyl-4-((1R,4R)-5-methyl-2,5-diazabicyclo[2.2.1]heptan-2-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((4-((1R,4R)-2,5-diazabicyclo[2.2.1]heptan-2-yl)-2-ethylphenyl)amino)-5-(trifluoromethyl)pyrimidin-4-yl)-4-cyclopropyl-3,4-dihydrothieno[2,3-f][1,4]thiazepin-5(2H)-one 1,1-dioxide. The title compound was isolated in 50% yield. MS (ESI) m/z: 633.4 [M+H]⁺. ¹H NMR (400 MHz, CDCl₃-d) δ, 8.65 (s, 1H), 8.01-7.96 (m, 1H), 7.31 (br d, J=9.2 Hz, 1H), 7.04 (br d, J=2.8 Hz, 1H), 6.51 (br s, 2H), 4.50-4.36 (m, 1H), 4.02-3.97 (m, 1H), 3.97-3.89 (m, 2H), 3.62 (br s, 2H), 3.61-3.56 (m, 2H), 3.16 (br d, J=10.0 Hz, 1H), 2.99-2.85 (m, 2H), 2.80 (br d, J=2.4 Hz, 2H), 2.60 (br d, J=7.6 Hz, 2H), 2.31-2.21 (m, 3H), 1.23-1.18 (m, 3H), 0.94-0.89 (m, 4H).

### Example 324: 7-(2-((4-((1R,5S)-3,8-diazabicyclo[3.2.1]octan-3-yl)-2-ethylphenyl)amino)-5-(trifluoromethyl)pyrimidin-4-yl)-4-cyclopropyl-3,4-dihydrothieno[2,3-f][1,4]thiazepin-5(2H)-one 1,1-dioxide

The title compound was prepared analogously to Example 319, where tert-butyl (1S,4S)-2,5-diazabicyclo[2.2.1]heptane-2-carboxylate was replaced with tert-butyl (1R,5S)-3,8-diazabicyclo[3.2.1]octane-8-carboxylate in step 1. The title compound was isolated. MS (ESI) m/z: 633.5 [M+H]⁺. ¹H NMR (400 MHz, CDCl₃) δ 8.66 (s, 1H), 8.03 (s, 1H), 7.63-7.36 (m, 1H), 7.06 (s, 1H), 6.82-6.71 (m, 2H), 4.07 (s, 2H), 3.93 (s, 2H), 3.63 (t, J=6.0 Hz, 2H), 3.54 (d, J=10.4 Hz, 2H), 3.31 (d, J=11.2 Hz, 2H), 2.91 (d, J=3.2, 6.4 Hz, 2H), 2.67-2.61 (m, 2H), 2.22-2.17 (m, 2H), 2.14-2.08 (m, 2H), 1.23 (t, J=7.6 Hz, 3H), 0.98-0.91 (m, 4H).

### Example 325: 4-cyclopropyl-7-(2-((2-ethyl-4-((1R,5S)-8-methyl-3,8-diazabicyclo[3.2.1]octan-3-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((4-((1R,5S)-3,8-diazabicyclo[3.2.1]octan-3-yl)-2-ethylphenyl)amino)-5-(trifluoromethyl)pyrimidin-4-yl)-4-cyclopropyl-3,4-dihydrothieno[2,3-f][1,4]thiazepin-5(2H)-one 1,1-dioxide. The title compound was isolated in 67% yield. MS (ESI) m/z: 647.4 [M+H]⁺. ¹H NMR (400 MHz, CDCl₃) δ 8.66 (s, 1H), 8.03 (s, 1H), 7.67-7.32 (m, 1H), 7.07-6.98 (m, 1H), 6.81-6.73 (m, 2H), 3.93 (s, 2H), 3.81-3.69 (m, 2H), 3.64 (t, J=6.0 Hz, 4H), 3.49 (d, J=11.6 Hz, 2H), 2.95-2.86 (m, 1H), 2.79-2.65 (m, 3H), 2.65-2.59 (m, 2H), 2.27-2.18 (m, 2H), 2.16-2.06 (m, 2H), 1.25-1.22 (m, 3H), 0.97-0.92 (m, 4H).

### Example 326: 4-cyclopropyl-7-(2-((2-cyclopropyl-4-((3S,5R)-3,5-dimethylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 308, where tert-butyl (R)-2-methylpiperazine-1-carboxylate was replaced with tert-butyl (2S,6R)-2,6-dimethylpiperazine-1-carboxylate in step 1. The title compound was isolated. MS (ESI) m/z: 647.2 [M+H]⁺. ¹H NMR (400 MHz, CDCl₃) δ ppm 8.69 (s, 1H) 8.05 (s, 1H) 7.54-7.98 (m, 2H) 6.75 (br d, J=2.0 Hz, 1H) 3.94 (t, J=6.0 Hz, 2H) 3.64 (t, J=6.0 Hz, 2H) 6.81-6.96 (m, 1H) 3.52 (brd, J=10.0 Hz, 2H) 3.04-3.17 (m, 2H) 2.86-2.98 (m, 1H) 2.30-2.46 (m, 2H) 1.89-1.93 (m, 1H) 1.21 (d, J=6.4 Hz, 6H) 1.00-1.08 (m, 2H) 0.92-0.99 (m, 4H) 0.84-0.92 (m, 1H) 0.69-0.75 (m, 2H).

### Example 327: 4-cyclopropyl-7-(2-((2-cyclopropyl-4-((3S,5S)-3,5-dimethylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 308, where tert-butyl (R)-2-methylpiperazine-1-carboxylate was replaced with tert-butyl (2S,6S)-2,6-dimethylpiperazine-1-carboxylate in step 1. The title compound was isolated. MS (ESI) m/z: 647.3 [M+H]⁺. ¹H NMR (400 MHz, DMSO-d₆) δ=9.74 (s, 1H), 8.89-8.64 (m, 1H), 7.85-7.71 (m, 1H), 7.28-7.00 (m, 1H), 6.73 (br d, J=6.8 Hz, 1H), 6.43 (br s, 1H), 4.01-3.70 (m, 4H), 3.17 (br d, J=5.2 Hz, 2H), 3.10 (dd, J=3.2, 11.3 Hz, 2H), 2.91-2.81 (m, 1H), 2.72 (dd, J=6.4, 11.4 Hz, 2H), 1.95-1.88 (m, 1H), 1.10 (d, J=6.4 Hz, 6H), 0.83-0.76 (m, 6H), 0.65-0.57 (m, 2H).

### Example 328: 4-cyclopropyl-7-(2-((2-cyclopropyl-4-((3S,5S)-3,4,5-trimethylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 4-cyclopropyl-7-(2-((2-cyclopropyl-4-((3S,5S)-3,5-dimethylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide. The title compound was isolated in 98% yield. MS (ESI) m/z: 661.2 [M+H]⁺. ¹H NMR (400 MHz, CDCl₃) δ=8.73-8.65 (m, 1H), 8.05 (s, 1H), 7.94-7.69 (m, 1H), 7.30-7.27 (m, 1H), 6.93-6.81 (m, 1H), 6.71 (br s, 1H), 3.94 (brt, J=5.6 Hz, 2H), 3.64 (t, J=6.0 Hz, 2H), 3.30-3.21 (m, 2H), 3.08-2.85 (m, 5H), 2.39 (s, 3H), 1.90 (br s, 1H), 1.15 (d, J=6.0 Hz, 6H), 1.05-1.01 (m, 2H), 0.98-0.93 (m, 4H), 0.74-0.69 (m, 2H).

### Example 329: 7-(2-((4-((1R,4R)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide

The title compound was prepared analogously to Example 305, steps 1-3, where tert-butyl (1S,4S)-2,5-diazabicyclo[2.2.1]heptane-2-carboxylate was replaced with tert-butyl (1R,4R)-2,5-diazabicyclo[2.2.1]heptane-2-carboxylate in step 1 and Example 310, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (1R,4R)-5-(4-((4-chloro-5-(trifluoromethyl)pyrimidin-2-yl)amino)-3-cyclopropylphenyl)-2,5-diazabicyclo[2.2.1]heptane-2-carboxylate. The title compound was isolated. MS (ESI) m/z: 631.4 [M+H]⁺. ¹H NMR (400 MHz, methanol-d₄): δ 8.76-8.56 (m, 1H), 7.96 (s, 1H), 7.40-7.13 (m, 1H), 6.52 (d, J=7.2 Hz, 1H), 6.28 (s, 1H), 4.74-4.50 (m, 1H), 3.99-3.88 (m, 3H), 3.76 (s, 2H), 3.64 (d, J=8.4 Hz, 1H), 3.15-3.02 (m, 3H), 2.90 (s, 1H), 2.07-1.86 (m, 3H), 0.92-0.84 (m, 6H), 0.70-0.60 (m, 2H).

### Example 330: 4-cyclopropyl-7-(2-((2-cyclopropyl-4-((1R,4R)-5-methyl-2,5-diazabicyclo[2.2.1]heptan-2-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((4-((1R,4R)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide. The title compound was isolated in 77% yield. MS (ESI) m/z: 645.4[M+H]⁺. ¹H NMR (400 MHz, methanol-d₄): δ 8.78-8.55 (m, 1H), 7.95 (s, 1H), 7.37-7.17 (m, 1H), 6.57 (d, J=6.4 Hz, 1H), 6.31 (s, 1H), 4.81-4.63 (m, 1H), 4.19-4.03 (m, 1H), 3.95 (s, 2H), 3.80-3.65 (m, 3H), 3.40-3.36 (m, 1H), 3.22-3.08 (m, 1H), 2.91-2.77 (m, 3H), 2.33-2.11 (m, 2H), 2.01-1.89 (m, 3H), 0.94-0.86 (m, 6H), 0.69-0.63 (m, 2H).

### Example 331: (S)-4-cyclopropyl-7-(2-((2-cyclopropyl-4-(3-methylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 308, where tert-butyl (R)-2-methylpiperazine-1-carboxylate was replaced with tert-butyl (S)-2-methylpiperazine-1-carboxylate. The title compound was isolated. MS (ESI) m/z: 633.2 [M+H]⁺. ¹H NMR (400 MHz, CDCl₃): δ 8.70 (s, 1H), 8.06 (s, 1H), 7.98-7.83 (m, 1H), 7.80-7.58 (m, 1H), 6.97-6.84 (m, 1H), 6.76 (s, 1H), 3.94 (t, J=5.6 Hz, 2H), 3.65 (t, J=5.6 Hz, 2H), 3.53 (d, J=12.2 Hz, 2H), 3.25-3.16 (m, 1H), 3.15-3.02 (m, 2H), 2.95-2.89 (m, 1H), 2.86-2.77 (m, 1H), 2.49 (t, J=10.6 Hz, 1H), 1.96-1.90 (m, 1H), 1.23 (d, J=6.4 Hz, 3H), 1.06-1.00 (m, 2H), 0.99-0.92 (m, 4H), 0.75-0.68 (m, 2H).

### Example 332: (S)-4-cyclopropyl-7-(2-((2-cyclopropyl-4-(3,4-dimethylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (S)-4-cyclopropyl-7-(2-((2-cyclopropyl-4-(3-methylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide. The title compound was isolated in 18% yield. MS (ESI) m/z: 647.2 [M+H]⁺. ¹H NMR (400 MHz, CDCl₃): δ 8.69 (s, 1H), 8.05 (s, 1H), 7.99-7.80 (m, 1H), 7.79-7.50 (m, 1H), 6.98-6.81 (m, 1H), 6.75 (d, J=1.4 Hz, 1H), 3.94 (t, J=5.6 Hz, 2H), 3.64 (t, J=6.2 Hz, 2H), 3.56-3.49 (m, 1H), 3.49-3.42 (m, 1H), 2.96-2.88 (m, 3H), 2.59-2.50 (m, 1H), 2.48-2.40 (m, 1H), 2.36 (s, 3H), 2.31-2.22 (m, 1H), 1.97-1.77 (m, 1H), 1.17 (d, J=6.4 Hz, 3H), 1.07-1.00 (m, 2H), 0.99-0.92 (m, 4H), 0.72 (d, J=4.6 Hz, 2H).

### Example 333: 7-(2-((4-((1R,4R)-2,5-diazabicyclo[2.2.1]heptan-2-yl)-2-cyclopropylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 310, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (1R,4R)-5-(4-amino-3-cyclopropylphenyl)-2,5-diazabicyclo[2.2.1]heptane-2-carboxylate. The title compound was isolated. MS (ESI) m/z: 647.1 [M+H]⁺. ¹H NMR (400 MHz, CDCl₃): δ 8.68 (s, 1H), 8.07 (s, 1H), 7.73-7.60 (m, 1H), 7.58-7.38 (m, 1H), 6.57-6.45 (m, 1H), 6.32 (s, 1H), 5.46 (m, 1H), 5.02 (t, J=7.2 Hz, 2H), 4.73 (t, J=6.8 Hz, 2H), 4.38 (s, 1H), 4.21-3.97 (m, 2H), 3.77 (m, 3H), 3.68 (d, J=9.6 Hz, 1H), 3.20-2.90 (m, 3H), 2.06-1.99 (m, 3H), 0.98 (d, J=8.4 Hz, 2H), 0.74-0.65 (m, 2H).

### Example 334: 7-(2-((2-cyclopropyl-4-((1R,4R)-5-methyl-2,5-diazabicyclo[2.2.1]heptan-2-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((4-((1R,4R)-2,5-diazabicyclo[2.2.1]heptan-2-yl)-2-cyclopropylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 51% yield MS (ESI) m/z: 661.3 [M+H]⁺. ¹H NMR (400 MHz, methanol-d₄): δ 8.83-8.59 (m, 1H), 7.98 (s, 1H), 7.42-7.20 (m, 1H), 6.57 (s, 1H), 6.32 (s, 1H), 5.36-5.15 (m, 1H), 4.96-4.88 (m, 2H), 4.70-4.56 (m, 1H), 4.06-3.99 (m, 2H), 3.89 (d, J=4.8 Hz, 2H), 3.76-3.70 (m, 2H), 3.63-3.43 (m, 1H), 3.40-3.33 (m, 2H), 3.27-3.18 (m, 1H), 3.03-2.73 (m, 3H), 2.33-2.24 (m, 2H), 2.08-1.97 (m, 1H), 1.94-1.90 (m, 1H), 0.89-0.86 (m, 2H), 0.68-0.62 (m, 2H).

### Example 335: 7-(2-((4-((1S,4S)-2,5-diazabicyclo[2.2.1]heptan-2-yl)-2-ethylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 310, where with tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (1S,4S)-5-(4-amino-3-ethylphenyl)-2,5-diazabicyclo[2.2.1]heptane-2-carboxylate. The title compound was isolated. MS (ESI) m/z: 635.4 [M+H]⁺. ¹H NMR (400 MHz, CDCl₃) δ 8.67 (s, 1H), 8.04 (br s, 1H), 7.33 (d, J=9.2 Hz, 1H), 7.04 (br s, 1H), 6.49 (s, 2H), 5.60-5.41 (m, 1H), 5.01 (br t, J=7.2 Hz, 2H), 4.73-4.62 (m, 2H), 4.49 (br s, 1H), 4.39-4.25 (m, 1H), 4.13-4.02 (m, 2H), 3.82-3.70 (m, 3H), 3.36 (br d, J=5.6 Hz, 3H), 2.62-2.56 (m, 2H), 2.23-2.14 (m, 2H), 1.19 (br t, J=7.6 Hz, 3H).

### Example 336: 7-(2-((4-((1R,4R)-2,5-diazabicyclo[2.2.1]heptan-2-yl)-2-ethylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 310, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (1R,4R)-5-(4-amino-3-ethylphenyl)-2,5-diazabicyclo[2.2.1]heptane-2-carboxylate. The title compound was isolated. MS (ESI) m/z: 635.3 [M+H]⁺. ¹H NMR (400 MHz, CDCl₃) δ, 8.66 (s, 1H), 8.06 (br d, J=2.0 Hz, 1H), 7.34-7.29 (m, 1H), 7.04-6.90 (m, 1H), 6.51-6.44 (m, 2H), 5.58-5.47 (m, 1H), 5.08-4.96 (m, 2H), 4.78-4.67 (m, 2H), 4.41-4.33 (m, 1H), 4.19-4.01 (m, 2H), 3.96-3.66 (m, 4H), 3.24-3.08 (m, 2H), 3.08-3.01 (m, 1H), 2.63-2.53 (m, 2H), 2.05-1.95 (m, 2H), 1.21 (br t, J=7.6 Hz, 3H).

### Example 337: 7-(2-((4-((1R,5S)-3,8-diazabicyclo[3.2.1]octan-3-yl)-2-ethylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 319, steps 1-4, where tert-butyl (1S,4S)-2,5-diazabicyclo[2.2.1]heptane-2-carboxylate was replaced with tert-butyl (1R,5S)-3,8-diazabicyclo[3.2.1]octane-8-carboxylate and Example 310, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (1R,5S)-3-(4-((4-chloro-5-(trifluoromethyl)pyrimidin-2-yl)amino)-3-ethylphenyl)-3,8-diazabicyclo[3.2.1]octane-8-carboxylate. The title compound was isolated. MS (ESI) m/z: 649.4 [M+H]⁺. ¹H NMR (400 MHz, CDCl₃) δ 8.67 (s, 1H), 8.07 (s, 1H), 7.60-7.28 (m, 1H), 7.02 (d, J=2.4, 7.6 Hz, 1H), 6.77-6.71 (m, 2H), 5.60-5.48 (m, 1H), 5.02 (t, J=7.6 Hz, 2H), 4.73 (t, J=6.8 Hz, 2H), 4.11 (s, 2H), 3.86 (s, 2H), 3.77 (d, J=4.8 Hz, 2H), 3.54-3.47 (m, 2H), 3.13 (d, J=11.2 Hz, 2H), 2.63 (q, J=7.6 Hz, 2H), 2.05 (s, 4H), 1.22 (t, J=7.6 Hz, 3H).

### Example 338: 7-(2-((4-(3,6-diazabicyclo[3.1.1]heptan-3-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide

The title compound was prepared analogously to Example 329, where tert-butyl (1R,4R)-2,5-diazabicyclo[2.2.1]heptane-2-carboxylate was replaced with tert-butyl 3,6-diazabicyclo[3.1.1]heptane-6-carboxylate. The title compound was isolated. MS (ESI) m/z: 631.4 [M+H]⁺. ¹H NMR (400 MHz, DMSO-d₆) δ 8.67 (s, 1H), 8.04 (s, 1H), 7.88-7.41 (m, 2H), 6.73-6.60 (m, 1H), 6.50 (s, 1H), 4.04-3.89 (m, 4H), 3.67-3.56 (m, 6H), 2.97-2.87 (m, 1H), 2.85-2.77 (m, 1H), 1.96-1.93 (m, 1H), 1.70-1.61 (m, 1H), 1.33-1.22 (m, 1H), 1.03-0.91 (m, 6H), 0.75-0.68 (m, 2H).

### Example 339: 4-cyclopropyl-7-(2-((2-cyclopropyl-4-(6-methyl-3,6-diazabicyclo[3.1.1]heptan-3-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((4-(3,6-diazabicyclo[3.1.1]heptan-3-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide. The title compound was isolated in 90% yield. MS (ESI) m/z: 645.2 [M+H]⁺. ¹H NMR (400 MHz, CDCl₃) δ 8.68 (s, 1H), 8.04 (s, 1H), 7.94-7.43 (m, 2H), 6.78-6.64 (m, 1H), 6.53 (s, 1H), 4.15-4.00 (m, 2H), 3.98-3.87 (m, 2H), 3.77-3.62 (m, 4H), 3.61-3.46 (m, 2H), 2.97-2.84 (m, 2H), 2.70 (s, 3H), 1.97-1.89 (m, 1H), 1.87-1.76 (m, 1H), 1.06-0.91 (m, 6H), 0.76-0.69 (m, 2H).

### Example 340: 7-(2-((4-(3,6-diazabicyclo[3.1.1]heptan-3-yl)-2-cyclopropylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 329, where tert-butyl (1R,4R)-2,5-diazabicyclo[2.2.1]heptane-2-carboxylate was replaced with tert-butyl 3,6-diazabicyclo[3.1.1]heptane-6-carboxylate in step 1 and 7-bromo-4-cyclopropyl-3,4-dihydrothieno[2,3-f][1,4]thiazepin-5(2H)-one 1,1-dioxide was replaced with 7-iodo-4-(oxetan-3-yl)-3,4-dihydrothieno[2,3-f][1,4]thiazepin-5(2H)-one 1,1-dioxide in step 7. The title compound was isolated. MS (ESI) m/z: 647.4 [M+H]⁺. ¹H NMR (400 MHz, CDCl₃) δ 8.68 (s, 1H), 8.08 (s, 1H), 7.86-7.41 (m, 2H), 6.66 (d, J=8.4 Hz, 1H), 6.49 (s, 1H), 5.57-5.48 (m, 1H), 5.02 (t, J=7.6 Hz, 2H), 4.73 (t, J=6.8 Hz, 2H), 4.17-4.00 (m, 2H), 3.94-3.86 (m, 2H), 3.81-3.72 (m, 2H), 3.62-3.50 (m, 4H), 2.82-2.68 (m, 1H), 1.97-1.89 (m, 1H), 1.68-1.60 (m, 2H), 1.03-0.96 (m, 2H), 0.75-0.66 (m, 2H).

### Example 341: (S)-7-(2-((2-Cyclopropyl-4-(3-methylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 308, steps 1-3, where tert-butyl (R)-2-methylpiperazine-1-carboxylate was replaced with tert-butyl (S)-2-methylpiperazine-1-carboxylate and Example 310, steps 1-3, where with tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (S)-4-(4-((4-chloro-5-(trifluoromethyl)pyrimidin-2-yl)amino)-3-cyclopropylphenyl)-2-methylpiperazine-1-carboxylate, Example 310, steps 4-5, where 1-((1S,4S)-5-(3-cyclopropyl-4-((5-(trifluoromethyl)-4-(trimethylstannyl)pyrimidin-2-yl)amino)phenyl)-2,5-diazabicyclo[2.2.1]heptan-2-yl)-2,2,2-trifluoroethan-1-one was replaced with (S)-1-(4-(3-cyclopropyl-4-((5-(trifluoromethyl)-4-(trimethylstannyl)pyrimidin-2-yl)amino)phenyl)-2-methylpiperazin-1-yl)-2,2,2-trifluoroethan-1-one in step 4. The title compound was isolated. MS (ESI) m/z: 649.1[M+H]⁺. ¹H NMR (400 MHz, CDCl₃): δ 8.71 (s, 1H), 8.09 (s, 1H), 7.96-7.81 (m, 1H), 7.72-7.45 (m, 1H), 6.94-6.85 (m, 1H), 6.78-6.69 (m, 1H), 5.59-5.50 (m, 1H), 5.03 (t, J=7.6 Hz, 2H), 4.74 (t, J=6.8 Hz, 2H), 4.12 (t, J=5.6 Hz, 2H), 3.78 (t, J=5.6 Hz, 2H), 3.51 (d, J=11.8 Hz, 2H), 3.20-2.93 (m, 3H), 2.79-2.64 (m, 1H), 2.36 (t, J=10.8 Hz, 1H), 1.94-1.82 (m, 1H), 1.16 (d, J=6.4 Hz, 3H), 1.08-0.97 (m, 2H), 0.77-0.65 (m, 2H).

### Example 342: (S)-7-(2-((2-cyclopropyl-4-(3,4-dimethylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306 where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (S)-7-(2-((2-cyclopropyl-4-(3-methylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 42% yield. MS (ESI) m/z: 663.1 [M+H]⁺. ¹H NMR (400 MHz, CDCl₃): δ 8.71 (s, 1H), 8.09 (s, 1H), 7.96-7.82 (m, 1H), 7.75-7.52 (m, 1H), 6.95-6.82 (m, 1H), 6.74 (s, 1H), 5.61-5.49 (m, 1H), 5.03 (t, J=7.6 Hz, 2H), 4.74 (t, J=6.8 Hz, 2H), 4.12 (t, J=5.6 Hz, 2H), 3.82-3.74 (m, 2H), 3.60-3.42 (m, 2H), 2.96-2.88 (m, 2H), 2.59-2.50 (m, 1H), 2.48-2.39 (m, 1H), 2.36 (s, 3H), 2.32-2.22 (m, 1H), 1.93-1.83 (m, 1H), 1.17 (d, J=6.0 Hz, 3H), 1.06-0.98 (m, 2H), 0.74-0.66 (m, 2H).

### Example 343: 4-Cyclopropyl-7-(2-((2-cyclopropyl-4-((3S,5R)-3,4,5-trimethylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306 where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 4-cyclopropyl-7-(2-((2-cyclopropyl-4-((3S,5R)-3,5-dimethylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide. The title compound was isolated in 42% yield. MS (ESI) m/z: 661.2 [M+H]⁺. ¹HNMR (400 MHz, CDCl₃) δ ppm 8.69 (s, 1H) 8.05 (s, 1H) 7.93 (br s, 1H) 7.71 (br s, 1H) 6.88 (br d, J=8.4 Hz, 1H) 3.84-4.04 (m, 2H) 3.64 (br t, J=6.0 Hz, 2H) 3.48 (br d, J=11.2 Hz, 2H) 2.81-3.02 (m, 1H) 2.73 (brs, 2H) 2.45 (brs, 4H) 1.80-1.96 (m, 2H) 1.19-1.35 (m, 7H) 0.88-1.09 (m, 7H) 0.62-0.81 (m, 2H).

### Example 344: 7-(2-((2-Cyclopropyl-4-((3S,5S)-3,5-dimethylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 341, where tert-butyl (S)-2-methylpiperazine-1-carboxylate was replaced with tert-butyl (2S,6S)-2,6-dimethylpiperazine-1-carboxylate. The title compound was isolated. MS (ESI) m/z: 663.3 [M+H]⁺. ¹H NMR (400 MHz, CDCl₃) δ=8.71 (s, 1H), 8.09 (s, 1H), 7.94-7.79 (m, 1H), 7.78-7.57 (m, 1H), 6.86 (br d, J=8.4 Hz, 1H), 6.71 (br s, 1H), 5.55 (quin, J=7.2 Hz, 1H), 5.03 (t, J=7.6 Hz, 2H), 4.74 (t, J=6.8 Hz, 2H), 4.12 (br t, J=5.6 Hz, 2H), 3.78 (t, J=5.6 Hz, 2H), 3.54-3.37 (m, 2H), 3.26 (dd, J=3.2, 11.6 Hz, 2H), 2.91 (dd, J=6.4, 11.6 Hz, 2H), 1.89-1.87 (m, 1H), 1.31 (d, J=6.8 Hz, 6H), 1.05-1.00 (m, 2H), 0.75-0.68 (m, 2H).

### Example 345: (R)-4-cyclopropyl-7-(2-((2-cyclopropyl-4-(3-(hydroxymethyl)piperazin-1-yl)phenyl)amino)-5-(trifluoromethyl)pyrimidin-4-yl)-3,4-dihydrothieno[2,3-f][1,4]thiazepin-5(2H)-one 1,1-dioxide

### Step 1: tert-butyl (R)-4-(3-cyclopropyl-4-nitrophenyl)-2-(hydroxymethyl)piperazine-1-carboxylate

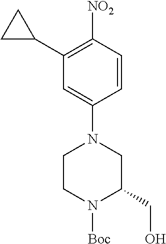 [SMILES: CC(C)(C)OC(=O)N1CCN(c2ccc([N+](=O)[O-])c(C3CC3)c2)C[C@@H]1CO]

The title compound was prepared analogously to Example 305, step 1, where tert-butyl (1S,4S)-2,5-diazabicyclo[2.2.1]heptane-2-carboxylate was replaced with tert-butyl (R)-2-(hydroxymethyl)piperazine-1-carboxylate. The title compound was isolated in 68% yield. MS (ESI) m/z: 378.2 [M+H]⁺

### Step 2: tert-butyl (R)-2-(acetoxymethyl)-4-(3-cyclopropyl-4-nitrophenyl)piperazine-1-carboxylate

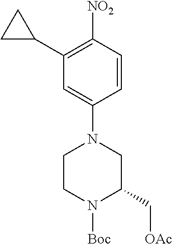 [SMILES: CC(=O)OC[C@H]1CN(c2ccc([N+](=O)[O-])c(C3CC3)c2)CCN1C(=O)OC(C)(C)C]

To a solution of tert-butyl (2R)-4-(3-cyclopropyl-4-nitro-phenyl)-2-(hydroxymethyl)piperazine-1-carboxylate (19 mmol) in dichloromethane (70 mL) was added triethylamine (37 mmol) and acetyl chloride (20 mmol). The mixture was stirred at 0° C. for 30 minutes, partitioned between ethyl acetate and water, the organic phase was separated, washed with brine, dried over anhydrous sodium sulfate, filtered, and concentrated under reduced pressure to afford a residue that was used in the next step without further purification. The title compound was isolated. MS (ESI) m/z: 420.2 [M+H]⁺.

### Steps 3-7: (R)-(4-(3-cyclopropyl-4-((4-(4-cyclopropyl-1,1-dioxido-5-oxo-2,3,4,5-tetrahydrothieno[2,3-f][,4]thiazepin-7-yl)-5-(trifluoromethyl)pyrimidin-2-yl)amino)phenyl)piperazin-2-yl)methyl acetate

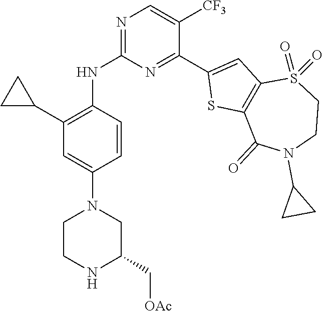 [SMILES: CC(=O)OC[C@H]1CN(c2ccc(Nc3ncc(C(F)(F)F)c(-c4cc5c(s4)C(=O)N(C4CC4)CCS5(=O)=O)n3)c(C3CC3)c2)CCN1]

The title compound was prepared analogously to Example 308, steps 2-6 where with tert-butyl (R)-4-(3-cyclopropyl-4-nitrophenyl)-2-methylpiperazine-1-carboxylate was replaced with tert-butyl (R)-2-(acetoxymethyl)-4-(3-cyclopropyl-4-nitrophenyl)piperazine-1-carboxylate. The title compound was isolated. MS (ESI) m/z: 691.3 [M+H]⁺.

### Step 8: (R)-4-cyclopropyl-7-(2-((2-cyclopropyl-4-(3-(hydroxymethyl)piperazin-1-yl)phenyl)amino)-5-(trifluoromethyl)pyrimidin-4-yl)-3,4-dihydrothieno[2,3-f][1,4]thiazepin-5(2H)-one 1,1-dioxide

To a solution of (R)-(4-(3-cyclopropyl-4-((4-(4-cyclopropyl-1,1-dioxido-5-oxo-2,3,4,5-tetrahydrothieno[2,3-f][11,4]thiazepin-7-yl)-5-(trifluoromethyl)pyrimidin-2-yl)amino)phenyl)piperazin-2-yl)methyl acetate (0.17 mmol) in ethyl alcohol (1 mL) and water (0.2 mL), was added potassium carbonate (0.51 mmol). The mixture was stirred at 50° C. for 12 hours. The reaction mixture was cooled down to room temperature, filtered and concentrated under reduced pressure to afford a residue that was purified by preparative TL. The title compound was isolated in 32% yield. MS (ESI) m/z: 649.3 [M+H]⁺. ¹H NMR (400 MHz, CDCl₃) δ ppm 8.69 (s, 1H) 8.05 (s, 1H) 7.48-7.96 (m, 2H) 6.89 (br d, J=8.0 Hz, 1H) 6.75 (d, J=2.4 Hz, 1H) 3.94 (br t, J=6.0 Hz, 2H) 3.74-3.82 (m, 1H) 3.59-3.71 (m, 3H) 3.51 (br d, J=11.6 Hz, 2H) 2.99-3.34 (m, 3H) 2.79-2.97 (m, 2H) 2.67 (brt, J=10.8 Hz, 1H) 1.82-1.92 (m, 1H) 1.18-1.32 (m, 2H) 0.99-1.07 (m, 2H) 0.93-0.98 (m, 3H) 0.65-0.75 (m, 2H).

### Example 346: 7-(2-((2-Ethyl-4-((1S,4S)-5-methyl-2,5-diazabicyclo[2.2.1]heptan-2-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((4-((1S,4S)-2,5-diazabicyclo[2.2.1]heptan-2-yl)-2-ethylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 83% yield. MS (ESI) m/z: 649.2 [M+H]⁺. ¹H NMR (400 MHz, CDCl₃) δ 8.66 (s, 1H), 8.05 (br s, 1H), 7.35 (br d, J=8.0 Hz, 1H), 7.03 (br s, 1H), 6.56-6.44 (m, 2H), 5.59-5.40 (m, 1H), 4.99 (br d, J=6.8 Hz, 2H), 4.74 (q, J=7.2 Hz, 2H), 4.52-4.39 (m, 1H), 4.16-3.95 (m, 3H), 3.85-3.72 (m, 3H), 3.69-3.54 (m, 2H), 3.41-3.28 (m, 1H), 2.76 (br d, J=1.6 Hz, 3H), 2.61 (q, J=7.6 Hz, 2H), 2.37-2.23 (m, 2H), 1.23-1.19 (m, 3H)

### Example 347: 7-(2-((2-ethyl-4-((1R,4R)-5-methyl-2,5-diazabicyclo[2.2.1]heptan-2-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced 7-(2-((4-((1R,4R)-2,5-diazabicyclo[2.2.1]heptan-2-yl)-2-ethylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 76% yield. MS (ESI) m/z: 649.2 [M+H]⁺. ¹H NMR (400 MHz, CDCl₃) δ=8.66 (s, 1H), 8.04 (br s, 1H), 7.31 (br d, J=9.6 Hz, 1H), 7.02 (br s, 1H), 6.51 (br s, 2H), 5.52-5.39 (m, 1H), 5.02-4.93 (m, 2H), 4.80-4.71 (m, 2H), 4.52-4.39 (m, 1H), 4.24-4.06 (m, 2H), 4.04-3.96 (m, 1H), 3.91-3.68 (m, 3H), 3.68-3.58 (m, 2H), 3.23-3.15 (m, 1H), 2.83-2.74 (m, 2H), 2.65-2.55 (m, 3H), 2.27-2.20 (m, 2H), 1.20 (brt, J=7.2 Hz, 3H).

### Example 348: 7-(2-((2-ethyl-4-((1R,5S)-8-methyl-3,8-diazabicyclo[3.2.1]octan-3-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((4-((1R,5S)-3,8-diazabicyclo[3.2.1]octan-3-yl)-2-ethylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 54% yield. MS (ESI) m/z: 663.4 [M+H]⁺. ¹H NMR (400 MHz, CDCl₃) δ 8.68 (s, 1H), 8.07 (s, 1H), 7.51-7.36 (m, 1H), 7.07-7.00 (m, 1H), 6.79-6.73 (m, 2H), 5.59-5.47 (m, 1H), 5.02 (t, J=7.2 Hz, 2H), 4.74 (t, J=6.8 Hz, 2H), 4.10 (d, J=1.6 Hz, 2H), 3.80-3.70 (m, 4H), 3.67-3.54 (m, 2H), 3.52-3.47 (m, 2H), 2.80-2.66 (m, 3H), 2.65-2.60 (m, 2H), 2.26-2.09 (m, 4H), 1.23-1.20 (m, 3H).

### Example 349: 7-(2-((2-cyclopropyl-4-(6-methyl-3,6-diazabicyclo[3.1.1]heptan-3-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((4-(3,6-diazabicyclo[3.1.1]heptan-3-yl)-2-cyclopropylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 33% yield. MS (ESI) m/z: 661.3 [M+H]⁺. ¹H NMR (400 MHz, CDCl₃) δ 8.68 (s, 1H), 8.08 (s, 1H), 7.87-7.43 (m, 2H), 6.74-6.62 (m, 1H), 6.51 (br.s., 1H), 5.60-5.48 (m, 1H), 5.06-4.99 (m, 2H), 4.77-4.70 (m, 2H), 4.18-4.06 (m, 2H), 3.81-3.75 (m, 2H), 3.74-3.69 (m, 2H), 3.60-3.54 (m, 2H), 3.39-3.29 (m, 2H), 2.67-2.56 (m, 1H), 2.15 (s, 3H), 1.97-1.88 (m, 1H), 1.63-1.60 (m, 1H), 1.05-0.96 (m, 2H), 0.77-0.69 (m, 2H).

### Example 350: 7-(2-((2-cyclopropyl-4-((3S,5R)-3,5-dimethylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 344, where tert-butyl (2S,6S)-2,6-dimethylpiperazine-1-carboxylate was replaced with tert-butyl (2S,6R)-2,6-dimethylpiperazine-1-carboxylate. The title compound was isolated. MS (ESI) m/z: 663.3 [M+H]+¹H NMR (400 MHz, CDCl₃) δ ppm 8.71 (s, 1H) 8.09 (s, 1H) 7.81-7.96 (m, 1H) 7.65-7.78 (m, 1H) 6.88 (br d, J=8 Hz, 1H) 6.75 (br d, J=2.00 Hz, 1H) 5.55 (quin, J=6.8 Hz, 1H) 5.04 (t, J=7.60 Hz, 2H) 4.74 (t, J=6.8 Hz, 2H) 4.11 (br d, J=5.6 Hz, 2H) 3.78 (br t, J=6 Hz, 2H) 3.51 (br d, J=10 Hz, 2H) 3.04-3.16 (m, 2H) 2.37 (br t, J=10.8 Hz, 2H) 1.84-1.92 (m, 1H) 1.21 (br d, J=6.4 Hz, 6H) 1.00-1.06 (m, 2H) 0.88-0.92 (m, 1H) 0.69-0.74 (m, 2H).

### Example 351: 7-(2-((2-cyclopropyl-4-((3S,5S)-3,4,5-trimethylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((2-cyclopropyl-4-((3S,5S)-3,5-dimethylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated. MS (ESI) m/z: 677.2 [M+H]⁺. ¹H NMR (400 MHz, CDCl₃) δ=8.71 (s, 1H), 8.09 (s, 1H), 7.95-7.78 (m, 1H), 7.58 (s, 1H), 6.87 (br d, J=9.2 Hz, 1H), 6.71 (d, J=2.4 Hz, 1H), 5.61-5.46 (m, 1H), 5.03 (t, J=7.2 Hz, 2H), 4.74 (t, J=6.8 Hz, 2H), 4.12 (br t, J=6.0 Hz, 2H), 3.81-3.73 (m, 2H), 3.44-3.27 (m, 2H), 3.26-2.99 (m, 4H), 2.61-2.43 (m, 3H), 1.90-1.86 (m, 1H), 1.28-1.24 (m, 6H), 1.05-0.99 (m, 2H), 0.72-0.65 (m, 2H).

### Example 352: (R)-4-cyclopropyl-7-(2-((2-cyclopropyl-4-(3-(hydroxymethyl)-4-methylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (R)-(4-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)piperazin-2-yl)methyl acetate and Example 345, step 8, where (R)-(4-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)piperazin-2-yl)methyl acetate was replaced with (R)-(4-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)-1-methylpiperazin-2-yl)methyl acetate. The title compound was isolated in 26% yield. MS (ESI).m/z: 663.4 [M+H]⁺. ¹H NMR (400 MHz, CDCl₃) δ ppm 8.70 (s, 1H) 8.05 (s, 1H) 7.80-7.97 (m, 1H) 7.57-7.77 (m, 1H) 6.84-6.98 (m, 1H) 6.76 (d, J=2.4 Hz, 1H) 3.88-4.04 (m, 3H) 3.59-3.70 (m, 3H) 3.43-3.56 (m, 2H) 2.85-3.06 (m, 4H) 2.59 (td, J=11.6, 2.8 Hz, 1H) 2.35-2.48 (m, 4H) 1.89 (br d, J=5.6 Hz, 1H) 1.00-1.06 (m, 2H) 0.92-0.99 (m, 4H) 0.68-0.74 (m, 2H).

### Example 353: (S)-4-cyclopropyl-7-(2-((2-ethyl-4-(3-methylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 319, where tert-butyl (1S,4S)-2,5-diazabicyclo[2.2.1]heptane-2-carboxylate was replaced with tert-butyl (S)-2-methylpiperazine-1-carboxylate in step 1. The title compound was isolated. MS (ESI) m/z: 621.4 [M+H]⁺. ¹H NMR (400 MHz, CDCl₃) δ 8.67 (s, 1H), 8.03 (s, 1H), 7.55-7.51 (m, 1H), 7.06-7.01 (m, 1H), 6.87-6.82 (m, 2H), 3.93-3.92 (m, 2H), 3.64-3.57 (m, 4H), 3.22-3.19 (m, 1H), 3.12-3.00 (m, 2H), 2.93-2.90 (m, 2H), 2.65-2.53 (m, 3H), 1.24-1.22 (m, 3H), 0.97-0.89 (m, 7H).

### Example 354: (S)-7-(2-((2-ethyl-4-(3-methylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 319, steps 1-4, where tert-butyl (1S,4S)-2,5-diazabicyclo[2.2.1]heptane-2-carboxylate was replaced with tert-butyl (S)-2-methylpiperazine-1-carboxylate in step 1 and Example 341, steps 4-8, where tert-butyl (S)-4-(4-((4-chloro-5-(trifluoromethyl)pyrimidin-2-yl)amino)-3-cyclopropylphenyl)-2-methylpiperazine-1-carboxylate was replaced with tert-butyl (S)-4-(4-((4-chloro-5-(trifluoromethyl)pyrimidin-2-yl)amino)-3-ethylphenyl)-2-methylpiperazine-1-carboxylate. The title compound was isolated. MS (ESI) m/z: 637.2 [M+H]⁺. ¹H NMR (400 MHz, CDCl₃) δ 8.67 (s, 1H), 8.06 (s, 1H), 7.54-7.46 (m, 1H), 7.07 (s, 1H), 6.85 (d, J=2.4 Hz, 2H), 5.57-5.50 (m, 1H), 5.02 (t, J=7.2 Hz, 2H), 4.73 (t, J=6.8 Hz, 2H), 4.10 (s, 2H), 3.77 (t, J=5.6 Hz, 2H), 3.57 (d, J=12.0 Hz, 2H), 3.21-3.17 (m, 1H), 3.16-2.99 (m, 2H), 2.82-2.79 (m, 1H), 2.64 (q, J=7.6 Hz, 2H), 2.45 (t, J=11.2 Hz, 1H), 2.15-2.14 (m, 1H), 1.26-1.25 (m, 3H), 1.21-1.19 (m, 3H).

### Example 355: (R)-7-(2-((2-ethyl-4-(3-(hydroxymethyl)piperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

### Steps 1-2: tert-butyl (R)-4-(3-ethyl-4-nitrophenyl)-2-(hydroxymethyl)piperazine-1-carboxylate

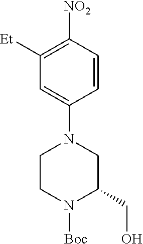 [SMILES: CCc1cc(N2CCN(C(=O)OC(C)(C)C)[C@@H](CO)C2)ccc1[N+](=O)[O-]]

The title compound was prepared analogously to Example 345, step 1, where 2-cyclopropyl-4-fluoro-1-nitrobenzene and tert-butyl (R)-2-(hydroxymethyl)piperazine-1-carboxylate were replaced with 2-ethyl-4-fluoro-1-nitrobenzene and tert-butyl (R)-2-(hydroxymethyl)piperazine-1-carboxylate. The title compound was isolated. MS (ESI) m/z: 366.2 [M+H]⁺

### Step 3: tert-butyl (R)-2-(acetoxymethyl)-4-(3-ethyl-4-nitrophenyl)piperazine-1-carboxylate

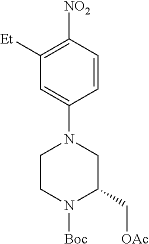 [SMILES: CCc1cc(N2CCN(C(=O)OC(C)(C)C)[C@@H](COC(C)=O)C2)ccc1[N+](=O)[O-]]

The title compound was prepared analogously to Example 345, step 2, where tert-butyl (R)-4-(3-cyclopropyl-4-nitrophenyl)-2-(hydroxymethyl)piperazine-1-carboxylate was replaced with tert-butyl (R)-4-(3-ethyl-4-nitrophenyl)-2-(hydroxymethyl)piperazine-1-carboxylate. The title compound was isolated in 89% yield. MS (ESI) m/z: 408.3 [M+H]⁺.

### Steps 4-5: tert-butyl (R)-2-(acetoxymethyl)-4-(4-((4-chloro-5-(trifluoromethyl)pyrimidin-2-yl)amino)-3-ethylphenyl)piperazine-1-carboxylate

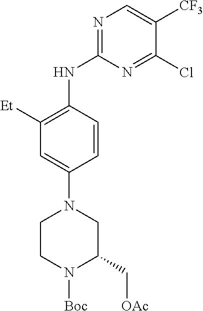 [SMILES: CCc1cc(N2CCN(C(=O)OC(C)(C)C)[C@@H](COC(C)=O)C2)ccc1Nc1ncc(C(F)(F)F)c(Cl)n1]

The title compound was prepared analogously to Example 319, steps 3-4, where tert-butyl (1S,4S)-5-(4-nitro-3-vinylphenyl)-2,5-diazabicyclo[2.2.1]heptane-2-carboxylate was replaced with tert-butyl (R)-2-(acetoxymethyl)-4-(3-ethyl-4-nitrophenyl)piperazine-1-carboxylate. The title compound was isolated. MS (ESI) m/z: 558.1 [M+H]⁺. ¹H NMR (400 MHz, DMSO-d₆) δ 9.92 (s, 1H), 8.80-8.37 (m, 1H), 7.09 (d, J=8.4 Hz, 1H), 6.85 (s, 1H), 6.79 (d, J=8.8 Hz, 1H), 4.47-4.30 (m, 2H), 4.29-4.19 (m, 1H), 3.88 (d, J=12.0 Hz, 1H), 3.72-3.50 (m, 2H), 3.18 (s, 1H), 2.79 (d, J=10.4 Hz, 1H), 2.70-2.56 (m, 1H), 2.49-2.45 (m, 2H), 2.01 (s, 3H), 1.42 (s, 9H), 1.07 (t, J=7.6 Hz, 3H).

### Steps 6-9: (R)-(4-(3-ethyl-4-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)phenyl)-1-(2,2,2-trifluoroacetyl)piperazin-2-yl)methyl acetate

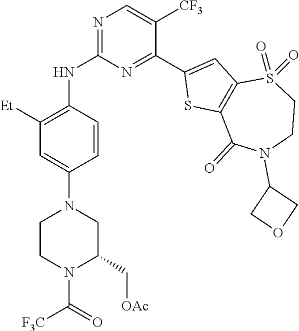 [SMILES: CCc1cc(N2CCN(C(=O)C(F)(F)F)[C@@H](COC(C)=O)C2)ccc1Nc1ncc(C(F)(F)F)c(-c2cc3c(s2)C(=O)N(C2COC2)CCS3(=O)=O)n1]

The title compound was prepared analogously to Example 310, steps 1-4, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (R)-2-(acetoxymethyl)-4-(4-((4-chloro-5-(trifluoromethyl)pyrimidin-2-yl)amino)-3-ethylphenyl)piperazine-1-carboxylate. The title compound was isolated. MS (ESI) m/z: 684.3 [M+H]⁺.

### Step 10: (R)-7-(2-((2-ethyl-4-(3-(hydroxymethyl)piperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 345, step 8, where (R)-(4-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)piperazin-2-yl)methyl acetate was replaced with (R)-(4-(3-ethyl-4-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)phenyl)-1-(2,2,2-trifluoroacetyl)piperazin-2-yl)methyl acetate. The title compound was isolated in 47% yield. MS (ESI) m/z: 653.2 [M+H]⁺. ¹H NMR (400 MHz, CDCl₃) δ 8.68 (s, 1H), 8.06 (s, 1H), 7.52 (d, J=2.8, 3.6 Hz, 1H), 7.10 (s, 1H), 6.93-6.79 (m, 2H), 5.61-5.41 (m, 1H), 5.01 (t, J=7.6 Hz, 2H), 4.73 (t, J=6.8 Hz, 2H), 4.09 (s, 2H), 4.04-3.97 (m, 1H), 3.95-3.87 (m, 1H), 3.77 (t, J=5.6 Hz, 2H), 3.70-3.61 (m, 2H), 3.58-3.51 (m, 1H), 3.49-3.41 (m, 1H), 3.32-3.20 (m, 2H), 3.14-3.08 (m, 1H), 2.64 (q, J=7.2 Hz, 2H), 1.23 (t, J=7.6 Hz, 3H).

### Example 356: 4-cyclopropyl-7-(2-((4-((3S,5S)-3,5-dimethylpiperazin-1-yl)-2-ethylphenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 319, where tert-butyl (1S,4S)-2,5-diazabicyclo[2.2.1]heptane-2-carboxylate was replaced with tert-butyl (2S,6S)-2,6-dimethylpiperazine-1-carboxylate in step 1. The title compound was isolated. MS (ESI) m/z: 635.4 [M+H]⁺

¹H NMR (400 MHz, methanol-d₄) δ=8.72-8.56 (m, 1H), 7.95 (s, 1H), 7.35-7.26 (m, 1H), 6.92 (d, J=2.0 Hz, 1H), 6.88 (d, J=8.8 Hz, 1H), 4.00-3.87 (m, 2H), 3.80-3.75 (m, 2H), 3.65-3.55 (m, 2H), 3.40-3.36 (m, 2H), 3.09-3.05 (m, 2H), 2.95-2.85 (m, 1H), 2.63 (q, J=7.6 Hz, 2H), 1.39 (d, J=6.8 Hz, 6H), 1.17 (t, J=7.6 Hz, 3H), 0.94-0.85 (m, 4H).

### Example 357: 7-(2-((2-cyclopropyl-4-((3S,5R)-3,4,5-trimethylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((2-cyclopropyl-4-((3S,5R)-3,5-dimethylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 70% yield. MS (ESI) m/z: 677.4 [M+H]⁺. ¹H NMR (400 MHz, CDCl₃) δ ppm 8.71 (s, 1H) 8.10 (s, 1H) 7.83-7.99 (m, 1H) 7.60-7.80 (m, 1H) 6.83-6.93 (m, 1H) 6.70-6.78 (m, 1H) 5.47-5.63 (m, 1H) 5.03 (t, J=7.60 Hz, 2H) 4.74 (t, J=6.80 Hz, 2H) 4.08-4.15 (m, 2H) 3.78 (t, J=6.00 Hz, 2H) 3.45-3.53 (m, 2H) 2.65-2.85 (m, 2H) 2.40-2.54 (m, 3H) 1.22-1.29 (m, 6H) 1.01-1.06 (m, 2H) 0.81-0.93 (m, 2H) 0.69-0.74 (m, 2H).

### Example 358: (R)-7-(2-((2-cyclopropyl-4-(3-(hydroxymethyl)piperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 308, steps 2-3, where with tert-butyl (R)-4-(3-cyclopropyl-4-nitrophenyl)-2-methylpiperazine-1-carboxylate was replaced with tert-butyl (R)-2-(acetoxymethyl)-4-(3-cyclopropyl-4-nitrophenyl)piperazine-1-carboxylate and Example 310, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (R)-2-(acetoxymethyl)-4-(4-((4-chloro-5-(trifluoromethyl)pyrimidin-2-yl)amino)-3-cyclopropylphenyl)piperazine-1-carboxylate. The title compound was isolated. MS (ESI).m/z: 665.2 [M+H]⁺. ¹H NMR (400 MHz, DMSO-d₆) δ ppm 9.80 (br s, 1H) 8.64-8.91 (m, 1H) 7.81 (br s, 1H) 7.06-7.28 (m, 1H) 6.78 (dd, J=8.8, 2.4 Hz, 1H) 6.49 (br s, 1H) 4.94-5.40 (m, 2H) 4.60-4.81 (m, 4H) 4.02 (br s, 4H) 3.46-3.73 (m, 4H) 3.14 (br d, J=11.6 Hz, 1H) 2.89-3.08 (m, 2H) 2.64-2.77 (m, 1H) 1.89-1.96 (m, 1H) 0.77-0.84 (m, 2H) 0.58-0.65 (m, 2H).

### Example 359: (R)-4-cyclopropyl-7-(2-((2-ethyl-4-(3-methylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 319, where tert-butyl (1S,4S)-2,5-diazabicyclo[2.2.1]heptane-2-carboxylate was replaced with tert-butyl (R)-2-methylpiperazine-1-carboxylate. The title compound was isolated. MS (ESI) m/z: 621.2 [M+H]⁺

¹H NMR (400 MHz, CDCl₃): δ 8.67 (s, 1H), 8.03 (s, 1H), 7.74-7.36 (m, 1H), 7.05 (s, 1H), 6.90-6.81 (m, 2H), 3.92 (d, J=5.2 Hz, 2H), 3.67-3.59 (m, 3H), 3.28-3.21 (m, 1H), 3.18-3.08 (m, 2H), 2.95-2.87 (m, 2H), 2.71-2.39 (m, 5H), 1.25 (m, 6H), 0.97-0.88 (m, 4H).

### Example 360: (R)-4-cyclopropyl-7-(2-((4-(3,4-dimethylpiperazin-1-yl)-2-ethylphenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (R)-4-cyclopropyl-7-(2-((2-ethyl-4-(3-methylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide. The title compound was isolated in 28% yield. MS (ESI) m/z: 635.2 [M+H]⁺

¹H NMR (400 MHz, CDCl₃): δ 8.66 (s, 1H), 8.03 (s, 1H), 7.78-7.34 (m, 1H), 7.12-7.00 (m, 1H), 6.87-6.81 (m, 2H), 3.93-3.82 (m, 2H), 3.63 (t, J=6.0 Hz, 2H), 3.59-3.54 (m, 1H), 3.52-3.48 (m, 1H), 3.02-2.88 (m, 3H), 2.67-2.60 (m, 3H), 2.54-2.46 (m, 1H), 2.40-2.30 (m, 4H), 1.26-1.19 (m, 6H), 0.98-0.92 (m, 4H).

### Example 361: (S)-4-cyclopropyl-7-(2-((4-(3,4-dimethylpiperazin-1-yl)-2-ethylphenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (S)-4-cyclopropyl-7-(2-((2-ethyl-4-(3-methylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide. The title compound was isolated in 50% yield. MS (ESI) m/z: 635.2 [M+H]⁺. ¹H NMR (400 MHz, CDCl₃) δ 8.67 (s, 1H), 8.03 (s, 1H), 7.57-7.52 (m, 1H), 7.05 (s, 1H), 6.86-6.84 (m, 2H), 3.93-3.92 (m, 2H), 3.65-3.52 (m, 4H), 3.37-3.11 (m, 2H), 2.92-2.91 (m, 1H), 2.67-2.62 (m, 1H), 2.65-2.62 (m, 4H), 2.52 (s, 3H), 1.29-1.27 (m, 3H), 1.24-1.22 (m, 3H), 0.97-0.93 (m, 4H).

### Example 362: (S)-7-(2-((4-(3,4-dimethylpiperazin-1-yl)-2-ethylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (S)-7-(2-((2-ethyl-4-(3-methylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 40% yield. MS (ESI) m/z: 651.2 [M+H]⁺. ¹H NMR (400 MHz, CDCl₃) δ 8.68 (s, 1H), 8.07 (s, 1H), 7.53-7.49 (m, 1H), 7.03-7.01 (m, 1H), 6.86-6.84 (m, 2H), 5.57-5.52 (m, 1H), 5.03 (t, J=7.6 Hz, 2H), 4.73 (t, J=6.8 Hz, 2H), 4.18-4.07 (m, 2H), 3.77 (t, J=5.6 Hz, 2H), 3.59-3.50 (m, 2H), 3.03-3.01 (m, 2H), 2.64 (q, J=7.6 Hz, 3H), 2.63-2.30 (m, 5H), 1.23 (m, J=7.6 Hz, 6H).

### Example 363: 4-cyclopropyl-7-(2-((2-ethyl-4-((3S,5S)-3,4,5-trimethylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 4-cyclopropyl-7-(2-((4-((3S,5S)-3,5-dimethylpiperazin-1-yl)-2-ethylphenyl)amino)-5-(trifluoromethyl)pyrimidin-4-yl)-3,4-dihydrothieno[2,3-f][1,4]thiazepin-5(2H)-one 1,1-dioxide. The title compound was isolated in 65% yield. MS (ESI) m/z: 649.4[M+H]⁺. 1H-NMR (400 MHz, methanol-d₄) δ 8.71-8.56 (m, 1H), 7.95 (s, 1H), 7.27-7.17 (m, 1H), 6.88-6.83 (m, 2H), 3.98-3.89 (m, 2H), 3.78-3.70 (m, 2H), 3.29-3.26 (m, 2H), 3.08-2.98 (m, 4H), 2.90-2.89 (m, 1H), 2.52 (q, J=7.6 Hz, 2H), 2.42 (s, 3H), 1.20-1.15 (m, 9H), 0.90-0.89 (m, 4H).

### Example 364: 7-(2-((2-ethyl-4-(piperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 319, steps 1-4, where tert-butyl (1S,4S)-2,5-diazabicyclo[2.2.1]heptane-2-carboxylate was replaced with tert-butyl piperazine-1-carboxylate and Example 341, steps 4-8, where tert-butyl (S)-4-(4-((4-chloro-5-(trifluoromethyl)pyrimidin-2-yl)amino)-3-cyclopropylphenyl)-2-methylpiperazine-1-carboxylate was replaced with tert-butyl 4-(4-((4-chloro-5-(trifluoromethyl)pyrimidin-2-yl)amino)-3-ethylphenyl)piperazine-1-carboxylate. The title compound was isolated. MS (ESI) m/z: 623.4 [M+H]⁺. ¹H NMR (400 MHz, CDCl₃) δ ppm 1.23 (t, J=7.6 Hz, 3H) 2.64 (q, J=7.6 Hz, 2H) 3.00-3.16 (m, 4H) 3.17-3.30 (m, 4H) 3.77 (brt, J=5.6 Hz, 2H) 4.11 (brs, 2H) 4.73 (t, J=6.8 Hz, 2H) 5.03 (t, J=7.6 Hz, 2H) 5.54 (quin, J=6.8 Hz, 1H) 6.85 (br d, J=2.4 Hz, 2H) 7.04 (br s, 1H) 7.39-7.61 (m, 1H) 8.07 (s, 1H) 8.68 (s, 1H).

### Example 365: (R)-7-(2-((2-cyclopropyl-4-(3-methylpiperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 155, step 3, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with tert-butyl (R)-4-(3-cyclopropyl-4-((5-(trifluoromethyl)-4-(trimethylstannyl)pyrimidin-2-yl)amino)phenyl)-2-methylpiperazine-1-carboxylate and 7-bromo-4-methyl-3,4-dihydrothieno[2,3-f][1,4]thiazepin-5(2H)-one 1,1-dioxide and Example 305, step 6, where tert-butyl (1S,4S)-5-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)-2,5-diazabicyclo[2.2.1]heptane-2-carboxylate was replaced with tert-butyl (R)-4-(3-cyclopropyl-4-((4-(4-methyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)-2-methylpiperazine-1-carboxylate. The title compound was isolated in 68% yield. MS (ESI) m/z: 707.2 [M+H]⁺. ¹H NMR (400 MHz, CDCl₃) δ=8.70 (s, 1H), 8.08 (s, 1H), 7.95-7.82 (m, 1H), 7.72-7.63 (m, 1H), 6.93-6.82 (m, 1H), 6.71 (d, J=2.4 Hz, 1H), 4.42-4.28 (m, 1H), 4.02-3.94 (m, 1H), 3.94-3.84 (m, 2H), 3.79-3.70 (m, 2H), 3.50 (d, J=10.8 Hz, 1H), 3.36 (d, J=12.0 Hz, 1H), 3.26 (s, 3H), 3.25-3.20 (m, 1H), 2.93 (dd, J=11.6, 3.6 Hz, 1H), 2.78-2.72 (m, 1H), 1.93-1.83 (m, 1H), 1.50 (s, 9H), 1.32 (d, J=6.8 Hz, 3H), 1.07-0.98 (m, 2H), 0.76-0.68 (m, 2H).

### Example 366: 4-cyclopropyl-7-(2-((4-((3S,5R)-3,5-dimethylpiperazin-1-yl)-2-ethylphenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 319, where tert-butyl (1S,4S)-2,5-diazabicyclo[2.2.1]heptane-2-carboxylate was replaced with tert-butyl (2S,6R)-2,6-dimethylpiperazine-1-carboxylate in step 1. The title compound was isolated. MS (ESI) m/z: 635.4 [M+H]⁺

¹H NMR (400 MHz, CDCl₃-d) δ, 8.69 (s, 1H), 8.06 (s, 1H), 7.66-7.48 (m, 1H), 7.12-7.02 (m, 1H), 6.94-6.84 (m, 2H), 3.95 (br s, 2H), 3.69-3.55 (m, 4H), 3.38-3.22 (m, 2H), 2.93 (br s, 1H), 2.77-2.58 (m, 4H), 1.34-1.25 (m, 9H), 0.96 (br d, J=5.2 Hz, 4H).

### Example 367: 4-cyclopropyl-7-(2-((2-ethyl-4-((3S,5R)-3,4,5-trimethylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 4-cyclopropyl-7-(2-((4-((3S,5R)-3,5-dimethylpiperazin-1-yl)-2-ethylphenyl)amino)-5-(trifluoromethyl)pyrimidin-4-yl)-3,4-dihydrothieno[2,3-f][1,4]thiazepin-5(2H)-one 1,1-dioxide. The title compound was isolated in 78% yield. MS (ESI) m/z: 649.3 [M+H]⁺. ¹H NMR (400 MHz, CDCl₃) δ=8.66 (s, 1H), 8.03 (s, 1H), 7.65-7.34 (m, 1H), 7.22-7.00 (m, 1H), 6.88-6.80 (m, 2H), 3.92 (br s, 2H), 3.63 (br t, J=5.8 Hz, 2H), 3.54-3.49 (m, 2H), 2.95-2.87 (m, 1H), 2.76-2.69 (m, 2H), 2.67-2.62 (m, 2H), 2.61-2.52 (m, 2H), 2.42 (s, 3H), 1.27-1.22 (m, 9H), 0.98-0.91 (m, 4H).

### Example 368: 7-(2-((4-((3S,5R)-3,5-dimethylpiperazin-1-yl)-2-ethylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

### Step 1: 4-chloro-N-(4-((3S,5R)-3,5-dimethylpiperazin-1-yl)-2-ethylphenyl)-5-(trifluoromethyl)pyrimidin-2-amine

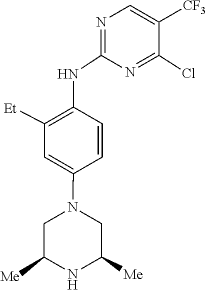 [SMILES: CCc1cc(N2C[C@@H](C)N[C@@H](C)C2)ccc1Nc1ncc(C(F)(F)F)c(Cl)n1]

The title compound was prepared analogously to Example 305, step 6, where tert-butyl (1S,4S)-5-(3-cyclopropyl-4-((4-(4-cyclopropyl-1,1-dioxido-5-oxo-2,3,4,5-tetrahydrothieno[2,3-f][1,4]thiazepin-7-yl)-5-(trifluoromethyl)pyrimidin-2-yl)amino)phenyl)-2,5-diazabicyclo[2.2.1]heptane-2-carboxylate was replaced with tert-butyl (2S,6R)-4-(4-((4-chloro-5-(trifluoromethyl)pyrimidin-2-yl)amino)-3-ethylphenyl)-2,6-dimethylpiperazine-1-carboxylate. The title compound was isolated. MS (ESI) m/z: 414.2 [M+H]⁺.

### Step 2: (9H-fluoren-9-yl)methyl (2S,6R)-4-(4-((4-chloro-5-(trifluoromethyl)pyrimidin-2-yl)amino)-3-ethylphenyl)-2,6-dimethylpiperazine-1-carboxylate

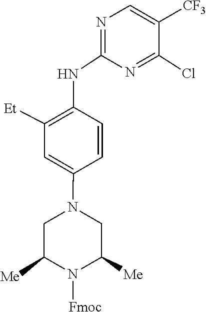

To a 0° C. solution of 4-chloro-N-(4-((3S,5R)-3,5-dimethylpiperazin-1-yl)-2-ethylphenyl)-5-(trifluoromethyl)pyrimidin-2-amine (2.42 mmol) and 9H-fluoren-9-ylmethyl carbonochloridate (3.62 mmol) in water (4 mL) and tetrahydrofuran (20 mL), was added potassium carbonate (12.1 mmol). The mixture was stirred for 30 minutes and the volatiles were removed under reduced pressure. The title compound was isolated and was used into the next step without further purification. MS (ESI) m/z: 636.2 [M+H]⁺.

### Step 3: (9H-Fluoren-9-yl)methyl (2S,6R)-4-(3-ethyl-4-((5-(trifluoromethyl)-4-(trimethylstannyl)pyrimidin-2-yl)amino)phenyl)-2,6-dimethylpiperazine-1-carboxylate

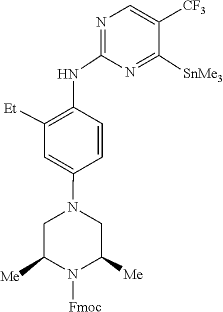

The title compound was prepared analogously to Example 4, step 1, where 1-(7-chloro-6-((4-chloro-5-(trifluoromethyl)pyrimidin-2-yl)amino)-3,4-dihydroisoquinolin-2(1H)-yl)-2,2,2-trifluoroethan-1-one was replaced with (9H-fluoren-9-yl)methyl (2S,6R)-4-(4-((4-chloro-5-(trifluoromethyl)pyrimidin-2-yl)amino)-3-ethylphenyl)-2,6-dimethylpiperazine-1-carboxylate. The title compound was isolated in 8% yield. MS (ESI) m/z: 766.3 [M+H]⁺.

### Step 4: (9H-fluoren-9-yl)methyl (2S,6R)-4-(3-ethyl-4-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)phenyl)-2,6-dimethylpiperazine-1-carboxylate

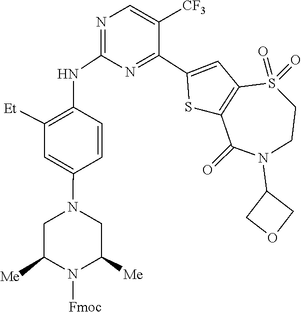

The title compound was prepared analogously to Example 155, step 3, where 1-(7-chloro-6-((5-(trifluoromethyl)-4-(trimethylstannyl)pyrimidin-2-yl)amino)-3,4-dihydroisoquinolin-2(1H)-yl)-2,2,2-trifluoroethan-1-one and 7-bromo-4-cyclopropyl-3,4-dihydrothieno[2,3-f][1,4]thiazepin-5(2H)-one 1,1-dioxide were replaced with (9H-Fluoren-9-yl)methyl (2S,6R)-4-(3-ethyl-4-((5-(trifluoromethyl)-4-(trimethylstannyl)pyrimidin-2-yl)amino)phenyl)-2,6-dimethylpiperazine-1-carboxylate and 7-iodo-4-(oxetan-3-yl)-3,4-dihydrothieno[2,3-f][1,4]thiazepin-5(2H)-one 1,1-dioxide. The title compound was isolated in 33% yield. MS (ESI) m/z: 873.2 [M+H]⁺.

### Step 5: 7-(2-((4-((3S,5R)-3,5-dimethylpiperazin-1-yl)-2-ethylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

To a solution of 9H-fluoren-9-ylmethyl (2S,6R)-4-[3-ethyl-4-[[4-[4-(oxetan-3-yl)-1,1,5-trioxo-2,3-dihydrothieno[2,3-f][1,4]thiazepin-7-yl]-5-(trifluoromethyl)pyrimidin-2-yl]amino]phenyl]-2,6-dimethyl-piperazine-1-carboxylate (0.02 mmol) in tetrahydrofuran (1 mL) was added piperidine (0.05 mmol). After 2 hours the volatiles were removed under reduced pressure and the residue was purified by preparative TLC (50% methanol in dichloromethane). The title compound was isolated in 92% yield. MS (ESI) m/z: 651.2 [M+H]⁺. ¹H NMR (400 MHz, CDCl₃-d) δ, 8.67 (s, 1H), 8.07 (s, 1H), 7.62-7.33 (m, 1H), 7.11-6.98 (m, 1H), 6.87-6.82 (m, 2H), 5.54 (br t, J=6.8 Hz, 1H), 5.03 (t, J=7.6 Hz, 2H), 4.77-4.71 (m, 2H), 4.10 (br s, 2H), 3.76 (br t, J=5.6 Hz, 2H), 3.60-3.52 (m, 2H), 3.08 (br s, 2H), 2.64 (q, J=7.6 Hz, 2H), 2.36 (t, J=11.2 Hz, 2H), 1.26-1.17 (m, 9H).

### Example 369: 7-(2-((2-ethyl-4-((3S,5R)-3,4,5-trimethylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((4-((3S,5R)-3,5-dimethylpiperazin-1-yl)-2-ethylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 87% yield. MS (ESI) m/z: 665.3 [M+H]⁺. ¹H NMR (400 MHz, CDCl₃-d) δ, 8.68 (s, 1H), 8.08-8.05 (m, 1H), 7.59-7.43 (m, 1H), 7.09 (br d, J=4.4 Hz, 1H), 6.86-6.82 (m, 2H), 5.52 (br s, 1H), 5.02 (br t, J=7.2 Hz, 2H), 4.74 (br t, J=6.8 Hz, 2H), 4.09 (br s, 2H), 3.76 (br s, 2H), 3.54 (br d, J=10.8 Hz, 2H), 2.89-2.81 (m, 2H), 2.80-2.68 (m, 2H), 2.64 (br d, J=7.6 Hz, 2H), 2.53 (br s, 3H), 1.33 (br d, J=4.4 Hz, 6H), 1.24-1.22 (m, 3H).

### Example 370: 7-(2-((4-((3S,5S)-3,5-dimethylpiperazin-1-yl)-2-ethylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 337, where tert-butyl (1R,5S)-3,8-diazabicyclo[3.2.1]octane-8-carboxylate was replaced with tert-butyl (2S,6S)-2,6-dimethylpiperazine-1-carboxylate. The title compound was isolated. MS (ESI) m/z: 651.4[M+H]⁺

¹H NMR (400 MHz, methanol-d₄) δ=8.70-8.64 (m, 1H), 8.03-7.93 (m, 1H), 7.29-7.26 (m, 1H), 6.92-6.86 (m, 2H), 5.32-5.27 (m, 1H), 4.91-4.87 (m, 4H), 4.15-3.95 (m, 2H), 3.92-3.80 (m, 2H), 3.61-3.56 (m, 2H), 3.39-3.35 (m, 2H), 3.08-3.04 (m, 2H), 2.66-2.60 (m, 2H), 1.40-1.38 (m, 6H), 1.19-1.15 (m, 3H).

### Example 371: (S)-4-(3-cyclopropyl-4-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)phenyl)piperazine-2-carboxamide

### Steps 1-3: 1-(tert-butyl) 2-Methyl (S)-4-(4-((4-chloro-5-(trifluoromethyl)pyrimidin-2-yl)amino)-3-cyclopropylphenyl)piperazine-1,2-dicarboxylate

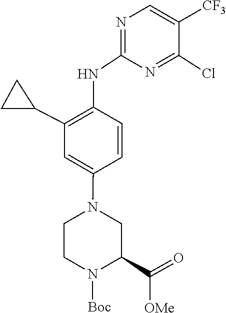 [SMILES: COC(=O)[C@@H]1CN(c2ccc(Nc3ncc(C(F)(F)F)c(Cl)n3)c(C3CC3)c2)CCN1C(=O)OC(C)(C)C]

The title compound was prepared analogously to Example 305, steps 1-3, where tert-butyl (1S,4S)-2,5-diazabicyclo[2.2.1]heptane-2-carboxylate was replaced with 1-(tert-butyl) 2-methyl (S)-piperazine-1,2-dicarboxylate. The title compound was isolated. MS (ESI) m/z: 556.4 [M+H]⁺. ¹H NMR (400 MHz, DMSO-d₆) δ 10.01 (s, 1H), 8.78-8.40 (m, 1H), 7.11 (d, J=8.4 Hz, 1H), 6.74 (d, J=2.0, 8.4 Hz, 1H), 6.45 (d, J=2.4 Hz, 1H), 4.87-4.59 (m, 1H), 3.99 (t, J=13.2 Hz, 1H), 3.80 (d, J=12.4 Hz, 1H), 3.68 (d, J=5.2 Hz, 3H), 3.62-3.50 (m, 1H), 3.23-3.03 (m, 1H), 2.91 (t, J=10.4 Hz, 1H), 2.76-2.58 (m, 1H), 1.96-1.79 (m, 1H), 1.48-1.34 (m, 9H), 0.86-0.76 (m, 2H), 0.58 (d, J=4.8 Hz, 2H).

### Steps 4-6: 1-((9H-Fluoren-9-yl)methyl) 2-methyl (S)-4-(3-cyclopropyl-4-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)phenyl)piperazine-1,2-dicarboxylate

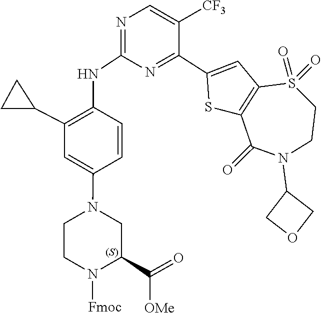

The title compound was prepared analogously to Example 368, steps 2-4, where 4-chloro-N-(4-((3S,5R)-3,5-dimethylpiperazin-1-yl)-2-ethylphenyl)-5-(trifluoromethyl)pyrimidin-2-amine was replaced with 1-(tert-butyl) 2-Methyl (S)-4-(4-((4-chloro-5-(trifluoromethyl)pyrimidin-2-yl)amino)-3-cyclopropylphenyl)piperazine-1,2-dicarboxylate. The title compound was isolated. MS (ESI) m/z: 915.5 [M+H]⁺.

### Step 7: (S)-4-(3-Cyclopropyl-4-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)phenyl)piperazine-2-carboxamide

To a solution of 1-((9H-fluoren-9-yl)methyl) 2-methyl (S)-4-(3-cyclopropyl-4-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)phenyl)piperazine-1,2-dicarboxylate (0.02 mmol) in acetonitrile (0.5 mL) was added ammonia in methanol (7 M, 3 mL). The mixture was stirred at 50° C. for 12 hours, cooled down to room temperature and the volatiles removed under reduced pressure. The residue was purified by preparative TLC (15% methanol in dichloromethane). The title compound was isolated in 43% yield. MS (ESI) m/z: 678.4 [M+H]⁺. ¹H NMR (400 MHz, DMSO-d₆) δ 9.77 (s, 1H), 8.91-8.63 (m, 1H), 7.82 (d, J=1.2 Hz, 1H), 7.33 (s, 1H), 7.24-7.08 (m, 2H), 6.76 (J=2.4, 8.8 Hz, 1H), 6.48 (s, 1H), 5.30-5.13 (m, 1H), 4.76-4.64 (m, 4H), 4.01 (d, J=1.2 Hz, 4H), 3.50 (J=2.4, 11.6 Hz, 1H), 3.01-2.91 (m, 1H), 2.77-2.66 (m, 3H), 2.49-2.47 (m, 2H), 2.36-2.30 (m, 1H), 0.84-0.79 (m, 2H), 0.64-0.58 (m, 2H).

### Example 372: (S)-4-(3-Cyclopropyl-4-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)phenyl)-1-methylpiperazine-2-carboxamide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (S)-4-(3-Cyclopropyl-4-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)phenyl)piperazine-2-carboxamide. The title compound was isolated in 43% yield. MS (ESI) m/z: 692.3 [M+H]⁺. ¹H NMR (400 MHz, DMSO-d₆) δ 9.76 (s, 1H), 8.94-8.63 (m, 1H), 7.92-7.75 (m, 1H), 7.32 (s, 1H), 7.14 (s, 2H), 6.76 (d, J=2.4, 8.8 Hz, 1H), 6.47 (s, 1H), 5.28-5.15 (m, 1H), 4.79-4.59 (m, 4H), 4.00 (d, J=2.4 Hz, 4H), 3.56 (d, J=11.2 Hz, 1H), 2.88 (d, J=11.2 Hz, 1H), 2.79-2.59 (m, 3H), 2.48-2.45 (m, 2H), 2.20 (s, 3H), 1.95-1.89 (m, 1H), 0.80 (d, J=1.6, 8.6 Hz, 2H), 0.65-0.59 (m, 2H)

### Example 373: (S)-4-(3-cyclopropyl-4-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)phenyl)-N-methylpiperazine-2-carboxamide

The title compound was prepared analogously to Example 371, where 1-((9H-fluoren-9-yl)methyl) 2-methyl (S)-4-(3-cyclopropyl-4-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)phenyl)piperazine-1,2-dicarboxylate was replaced with 1-((9H-fluoren-9-yl)methyl) 2-methyl (S)-4-(3-cyclopropyl-4-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)phenyl)piperazine-1,2-dicarboxylate. The title compound was isolated in 29% yield. MS (ESI) m/z: 692.3 [M+H]⁺. ¹H NMR (400 MHz, DMSO-d₆) δ 9.76 (s, 1H), 8.90-8.66 (m, 1H), 7.82 (d, J=4.0 Hz, 2H), 7.29-7.07 (m, 1H), 6.75 (d, J=2.4, 8.8 Hz, 1H), 6.49-6.41 (m, 1H), 5.27-5.16 (m, 1H), 4.78-4.67 (m, 4H), 4.00 (s, 4H), 3.50-3.43 (m, 1H), 2.98-2.92 (m, 1H), 2.79-2.67 (m, 3H), 2.63 (d, J=4.8 Hz, 3H), 2.48-2.48 (m, 2H), 1.97-1.86 (m, 1H), 0.83-0.78 (m, 2H), 0.60 (q, J=5.2 Hz, 2H).

### Example 374: (R)-7-(2-((4-cyclopropyl-6-(3-methylpiperazin-1-yl)pyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

### Step 1: 2-Chloro-4-cyclopropyl-5-nitropyridine

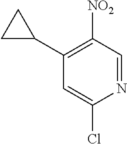 [SMILES: O=[N+]([O-])c1cnc(Cl)cc1C1CC1]

A mixture of 2,4-dichloro-5-nitropyridine (124 mmol), cyclopropylboronic acid (137 mmol), 1,1′-bis(diphenylphosphino)ferrocene)palladium(II) dichloride (6.22 mmol) and sodium carbonate (149 mmol) in toluene (288 mL) and water (96 mL) was stirred at 90° C. for 12 hours and cooled down to room temperature. Evaporation of volatiles under reduced pressure afforded a residue that was purified by silica gel column chromatography (5% ethyl acetate in hexanes) to afford the title compound in 59% yield. MS (ESI) m/z: 199.1 [M+H]⁺. ¹H NMR (400 MHz, CDCl₃-d) δ, 0.89-0.97 (m, 2H) 1.26-1.35 (m, 2H) 2.55 (tt, J=8.8, 5.2 Hz, 1H) 6.95 (s, 1H) 8.85 (s, 1H).

### Steps 2-9: (R)-7-(2-((4-cyclopropyl-6-(3-methylpiperazin-1-yl)pyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 305, steps 1-3, where 2-cyclopropyl-4-fluoro-1-nitrobenzene and tert-butyl (1S,4S)-2,5-diazabicyclo[2.2.1]heptane-2-carboxylate were replaced with 2-chloro-4-cyclopropyl-5-nitropyridine and tert-butyl (R)-2-methylpiperazine-1-carboxylate and Example 368, steps 1-5, where tert-butyl (2S,6R)-4-(4-((4-chloro-5-(trifluoromethyl)pyrimidin-2-yl)amino)-3-ethylphenyl)-2,6-dimethylpiperazine-1-carboxylate was replaced with tert-butyl (R)-4-(5-((4-chloro-5-(trifluoromethyl)pyrimidin-2-yl)amino)-4-cyclopropylpyridin-2-yl)-2-methylpiperazine-1-carboxylate. The title compound was isolated. MS (ESI) m/z: 650.2 [M+H]⁺. ¹H NMR (400 MHz, CDCl₃-d) δ, 0.72-0.78 (m, 2H) 0.99-1.08 (m, 2H) 1.27 (br d, J=6.0 Hz, 3H) 1.84-1.93 (m, 1H) 2.57-2.72 (m, 1H) 2.93-3.10 (m, 3H) 3.21 (br d, J=8.0 Hz, 1H) 3.77 (br t, J=5.2 Hz, 2H) 4.11 (br d, J=5.2 Hz, 3H) 4.15-4.22 (m, 1H) 4.73 (t, J=6.8 Hz, 2H) 5.03 (t, J=7.6 Hz, 2H) 5.48-5.59 (m, 1H) 6.28 (s, 1H) 7.18 (br s, 1H) 8.07 (s, 1H) 8.37-8.46 (m, 1H) 8.70 (s, 1H).

### Example 375: (R)-7-(2-((2-cyclopropyl-6-(3-methylpiperazin-1-yl)pyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 374, step 1, where 2,4-dichloro-5-nitropyridine was replaced with 2,6-dichloro-3-nitropyridine, Example 305, steps 1-3, where 2-cyclopropyl-4-fluoro-1-nitrobenzene, tert-butyl (1S,4S)-2,5-diazabicyclo[2.2.1]heptane-2-carboxylate and DMF were replaced with 6-chloro-2-cyclopropyl-3-nitropyridine, tert-butyl (R)-2-methylpiperazine-1-carboxylate and NMP in step 1 and Example 310, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (R)-4-(5-((4-chloro-5-(trifluoromethyl)pyrimidin-2-yl)amino)-6-cyclopropylpyridin-2-yl)-2-methylpiperazine-1-carboxylate. The title compound was isolated. MS (ESI) m/z: 650.4 [M+H]⁺. ¹H NMR (400 MHz, methanol-d₄) δ 8.72-8.55 (m, 1H), 7.98 (s, 1H), 7.49 (s, 1H), 6.64 (d, J=8.4 Hz, 1H), 5.28 (s, 1H), 4.90-4.86 (m, 4H), 4.24 (d, J=13.6 Hz, 2H), 4.10-3.95 (m, 2H), 3.90-3.80 (m, 2H), 3.30 (s, 1H), 3.20-3.18 (m, 1H), 3.10-3.06 (s, 1H), 2.99-2.91 (m, 2H), 2.65 (t, J=12.0 Hz, 1H), 2.14-2.07 (m, 1H), 1.24 (d, J=6.8 Hz, 3H), 1.04-0.95 (m, 2H), 0.85-0.82 (m, 2H).

### Example 376: (R)-7-(2-((2-cyclopropyl-6-(3,4-dimethylpiperazin-1-yl)pyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (R)-7-(2-((2-cyclopropyl-6-(3-methylpiperazin-1-yl)pyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 85% yield. MS (ESI) m/z: 664.4 [M+H]⁺. ¹H NMR (400 MHz, methanol-d₄) δ 8.77-8.57 (m, 1H), 7.98 (s, 1H), 7.47 (s, 1H), 6.60 (d, J=8.8 Hz, 1H), 5.38-5.25 (m, 1H), 4.89-4.86 (m, 4H), 4.17-4.01 (m, 4H), 3.94-3.80 (m, 2H), 3.01-2.89 (m, 2H), 2.62 (dd, J=12.8, 10.0 Hz, 1H), 2.42-2.30 (s, 4H), 2.28-2.21 (m, 1H), 2.12-2.06 (m, 1H), 1.17 (d, J=6.0 Hz, 3H), 1.04-0.98 (m, 2H), 0.85-0.80 (m, 2H).

### Example 377: 7-(2-((2-Cyclopropyl-4-(2,6-diazaspiro[3.3]heptan-2-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 305, steps 1-3, where tert-butyl (1S,4S)-2,5-diazabicyclo[2.2.1]heptane-2-carboxylate and DMF were replaced with tert-butyl 2,6-diazaspiro[3.3]heptane-2-carboxylate and NMP in step 1 and Example 310, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl 6-(4-((4-chloro-5-(trifluoromethyl)pyrimidin-2-yl)amino)-3-cyclopropylphenyl)-2,6-diazaspiro[3.3]heptane-2-carboxylate. The title compound was isolated. MS (ESI) m/z: 647.4 [M+H]⁺. ¹H NMR (400 MHz, CDCl₃) δ ppm 0.65-0.71 (m, 2H) 0.95-1.02 (m, 2H) 1.85-1.88 (m, 1H) 3.77 (brt, J=5.6 Hz, 2H) 3.82-3.95 (m, 3H) 3.99 (s, 4H) 4.02-4.20 (m, 3H) 4.74 (t, J=6.8 Hz, 2H) 5.03 (t, J=7.6 Hz, 2H) 5.48-5.60 (m, 1H) 6.21 (br s, 1H) 6.39 (br d, J=8.4 Hz, 1H) 7.37-7.81 (m, 2H) 8.08 (s, 1H) 8.68 (s, 1H).

### Example 378: (R)-7-(2-((2-cyclopropyl-4-(3,4-dimethylpiperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (R)-7-(2-((2-cyclopropyl-4-(3-methylpiperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 28% yield. MS (ESI) m/z: 621.4 [M+H]⁺. ¹H NMR (400 MHz, methanol-d₄) δ=8.74-8.52 (m, 1H), 7.98 (s, 1H), 7.51-7.30 (m, 1H), 6.87 (dd, J=8.4, 2.4 Hz, 1H), 6.65 (d, J=2.4 Hz, 1H), 3.95-3.89 (m, 2H), 3.87-3.82 (m, 2H), 3.56-3.48 (m, 2H), 3.18 (s, 3H), 2.98-2.92 (m, 1H), 2.85 (m, 1H), 2.52-2.42 (m, 2H), 2.36 (s, 3H), 2.35-2.34 (m, 1H), 2.00-1.89 (m, 1H), 1.18 (d, J=6.4 Hz, 3H), 0.92-0.86 (m, 2H), 0.68-0.59 (m, 2H)

### Example 379: 7-(2-((4-((1R,4R)-2,5-diazabicyclo[2.2.1]heptan-2-yl)-2-cyclopropylphenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 305, where tert-butyl (1S,4S)-2,5-diazabicyclo[2.2.1]heptane-2-carboxylate and DMF were replaced with tert-butyl (1R,4R)-2,5-diazabicyclo[2.2.1]heptane-2-carboxylate and NMP in step 1 and 7-bromo-4-cyclopropyl-3,4-dihydrothieno[2,3-f][1,4]thiazepin-5(2H)-one 1,1-dioxide was replaced with 7-bromo-4-methyl-3,4-dihydrothieno[2,3-f][1,4]thiazepin-5(2H)-one 1,1-dioxide in step 5. The title compound was isolated. MS (ESI) m/z: 605.2 [M+H]⁺. ¹H NMR (400 MHz, DMSO-d₆) δ 9.72 (s, 1H), 8.95-8.57 (m, 1H), 7.93-7.66 (m, 1H), 7.24-6.97 (m, 1H), 6.52-6.35 (m, 1H), 6.24-6.00 (m, 1H), 4.49 (br s, 1H), 4.05 (s, 1H), 4.00-3.81 (m, 4H), 3.55 (br d, J=8.0 Hz, 1H), 3.10-3.03 (m, 5H), 3.01-2.97 (m, 1H), 1.97 (br d, J=9.8 Hz, 1H), 1.93-1.85 (m, 1H), 1.78 (br d, J=10.0 Hz, 1H), 0.80 (br d, J=8.4 Hz, 2H), 0.61 (br s, 2H).

### Example 380: (S)-7-(2-((2-cyclopropyl-4-(3-methylpiperazin-1-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 308, steps 1-3, where tert-butyl (R)-2-methylpiperazine-1-carboxylate was replaced with tert-butyl (S)-2-methylpiperazine-1-carboxylate in step 1 and Example 310, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (S)-4-(4-((4-chloro-5-(trifluoromethyl)pyrimidin-2-yl)amino)-3-cyclopropylphenyl)-2-methylpiperazine-1-carboxylate. The title compound was isolated. MS (ESI) m/z: 607.2 [M+H]⁺. ¹H NMR (400 MHz, CDCl₃): δ 8.70 (s, 1H), 8.08 (s, 1H), 7.95-7.86 (m, 1H), 7.75-7.59 (m, 1H), 6.89 (d, J=7.6 Hz, 1H), 6.75 (d, J=2.4 Hz, 1H), 3.95-3.87 (m, 2H), 3.77-3.68 (m, 2H), 3.53 (d, J=12.0 Hz, 2H), 3.30 (s, 3H), 3.22-3.16 (m, 1H), 3.09 (m, 2H), 2.86-2.72 (m, 1H), 2.47 (t, J=10.8 Hz, 1H), 1.90-1.86 (m, 1H), 1.22 (d, J=6.4 Hz, 3H), 1.07-1.01 (m, 2H), 0.76-0.68 (m, 2H).

### Example 381: 7-(2-((4-(3,6-diazabicyclo[3.1.1]heptan-3-yl)-2-cyclopropylphenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 308, steps 1-3, where tert-butyl (R)-2-methylpiperazine-1-carboxylate was replaced with tert-butyl 3,6-diazabicyclo[3.1.1]heptane-6-carboxylate in step 1, and Example 305, steps 4-6, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl 3-(4-((4-chloro-5-(trifluoromethyl)pyrimidin-2-yl)amino)-3-cyclopropylphenyl)-3,6-diazabicyclo[3.1.1]heptane-6-carboxylate. The title compound was isolated. MS (ESI) m/z: 510.2 [M+H]⁺. ¹H NMR (400 MHz, CDCl₃) δ 8.68 (s, 1H), 8.06 (s, 1H), 7.83-7.42 (m, 2H), 6.73-6.64 (m, 1H), 6.50 (s, 1H), 4.12-4.01 (m, 2H), 3.94-3.86 (m, 2H), 3.76-3.70 (m, 2H), 3.68-3.59 (m, 4H), 3.24 (s, 3H), 2.93-2.81 (m, 1H), 1.95-1.90 (m, 1H), 1.03-0.98 (m, 2H), 0.93-0.79 (m, 2H), 0.74-0.69 (m, 2H).

### Example 382: 7-(2-((6-((1R,4R)-2,5-diazabicyclo[2.2.1]heptan-2-yl)-4-cyclopropylpyridin-3-yl)amino)-5-(trifluoromethyl)pyrimidin-4-yl)-4-methyl-3,4-dihydrothieno[2,3-f][1,4]thiazepin-5(2H)-one 1,1-dioxide

### Steps 1-4: tert-butyl (1R,4R)-5-(5-((4-chloro-5-(trifluoromethyl)pyrimidin-2-yl)amino)-4-cyclopropylpyridin-2-yl)-2,5-diazabicyclo[2.2.1]heptane-2-carboxylate

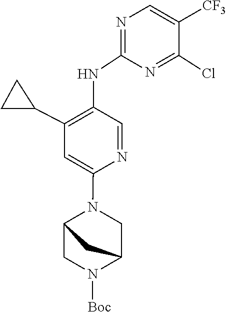 [SMILES: CC(C)(C)OC(=O)N1C[C@H]2C[C@@H]1CN2c1cc(C2CC2)c(Nc2ncc(C(F)(F)F)c(Cl)n2)cn1]

The title compound was prepared analogously to Example 374, steps 1-4, where tert-butyl (R)-2-methylpiperazine-1-carboxylate was replaced with tert-butyl (1R,4R)-2,5-diazabicyclo[2.2.1]heptane-2-carboxylate. The title compound was isolated. MS (ESI) m/z: 511.2 [M+H]⁺

### Steps 5-9: 7-(2-((6-((1R,4R)-2,5-diazabicyclo[2.2.1]heptan-2-yl)-4-cyclopropylpyridin-3-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 368, where tert-butyl (2S,6R)-4-(4-((4-chloro-5-(trifluoromethyl)pyrimidin-2-yl)amino)-3-ethylphenyl)-2,6-dimethylpiperazine-1-carboxylate was replaced with tert-butyl (1R,4R)-5-(5-((4-chloro-5-(trifluoromethyl)pyrimidin-2-yl)amino)-4-cyclopropylpyridin-2-yl)-2,5-diazabicyclo[2.2.1]heptane-2-carboxylate in step 1 and 7-iodo-4-(oxetan-3-yl)-3,4-dihydrothieno[2,3-f][1,4]thiazepin-5(2H)-one 1,1-dioxide was replaced with 7-bromo-4-methyl-3,4-dihydrothieno[2,3-f][1,4]thiazepin-5(2H)-one 1,1-dioxide. The title compound was isolated. MS (ESI) m/z: 361.3 [M+H]⁺. ¹H NMR (400 MHz, CDCl₃) δ ppm 8.67 (br s, 1H) 8.28 (s, 1H) 8.04 (br s, 1H) 7.27-7.30 (m, 1H) 5.94 (br t, J=6.4 Hz, 1H) 4.90 (br s, 1H) 4.31 (br d, J=1.6 Hz, 1H) 3.92 (br d, J=1.6 Hz, 2H) 3.75 (brd, J=1.6 Hz, 2H) 3.49-3.67 (m, 2H) 3.15-3.35 (m, 5H) 1.83-1.96 (m, 3H) 1.00 (brd, J=9.2 Hz, 2H) 0.76 (br d, J=5.2 Hz, 2H).

### Example 383: 7-(2-((6-(3,6-diazabicyclo[3.1.1]heptan-3-yl)-4-cyclopropylpyridin-3-yl)amino)-5-(trifluoromethyl)pyrimidin-4-yl)-4-methyl-3,4-dihydrothieno[2,3-f][1,4]thiazepin-5(2H)-one 1,1-dioxide

### Steps 1-3: tert-butyl 3-(5-((4-chloro-5-(trifluoromethyl)pyrimidin-2-yl)amino)-4-cyclopropylpyridin-2-yl)-3,6-diazabicyclo[3.1.1]heptane-6-carboxylate

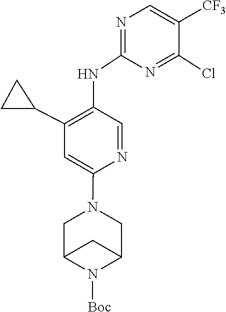 [SMILES: CC(C)(C)OC(=O)N1C2CC1CN(c1cc(C3CC3)c(Nc3ncc(C(F)(F)F)c(Cl)n3)cn1)C2]

The title compound was prepared analogously to Example 305, steps 1-3, where 2-cyclopropyl-4-fluoro-1-nitrobenzene and tert-butyl (1S,4S)-2,5-diazabicyclo[2.2.1]heptane-2-carboxylate were replaced with 2-chloro-4-cyclopropyl-5-nitropyridine and tert-butyl 3,6-diazabicyclo[3.1.1]heptane-6-carboxylate. The title compound was isolated. MS (ESI) m/z: 511.4 [M+H]⁺.

### Steps 4-6: 7-(2-((6-(3,6-diazabicyclo[3.1.1]heptan-3-yl)-4-cyclopropylpyridin-3-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 305, steps 4-6, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl 3-(5-((4-chloro-5-(trifluoromethyl)pyrimidin-2-yl)amino)-4-cyclopropylpyridin-2-yl)-3,6-diazabicyclo[3.1.1]heptane-6-carboxylate in step 4 and 7-bromo-4-cyclopropyl-3,4-dihydrothieno[2,3-f][1,4]thiazepin-5(2H)-one 1,1-dioxide was replaced with 7-bromo-4-methyl-3,4-dihydrothieno[2,3-f][1,4]thiazepin-5(2H)-one 1,1-dioxide in step 5. The title compound was isolated. MS (ESI) m/z: 606.1 [M+H]⁺. ¹H NMR (400 MHz, CDCl₃) δ 8.68 (s, 1H), 8.36 (s, 1H), 8.04 (s, 1H), 7.26-7.12 (m, 1H), 6.14 (s, 1H), 4.28-4.06 (m, 2H), 4.01-3.77 (m, 6H), 3.76-3.70 (m, 2H), 3.24 (s, 3H), 3.05-2.87 (m, 1H), 1.94-1.88 (m, 1H), 1.79-1.69 (m, 1H), 1.02 (q, J=6.4 Hz, 2H), 0.81-0.72 (m, 2H).

### Example 384: 7-(2-((6-(3,6-diazabicyclo[3.1.1]heptan-3-yl)-4-cyclopropylpyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 368, where tert-butyl (2S,6R)-4-(4-((4-chloro-5-(trifluoromethyl)pyrimidin-2-yl)amino)-3-ethylphenyl)-2,6-dimethylpiperazine-1-carboxylate was replaced with tert-butyl 3-(5-((4-chloro-5-(trifluoromethyl)pyrimidin-2-yl)amino)-4-cyclopropylpyridin-2-yl)-3,6-diazabicyclo[3.1.1]heptane-6-carboxylate in step 1. The title compound was isolated. MS (ESI) m/z: 648.2 [M+H]⁺. ¹H NMR (400 MHz, CDCl₃) δ 8.69 (d, J=3.6 Hz, 1H), 8.40 (s, 1H), 8.12-8.01 (m, 1H), 7.24-7.12 (m, 1H), 6.21-6.08 (m, 1H), 5.58-5.45 (m, 1H), 5.02 (t, J=7.2 Hz, 2H), 4.75-4.68 (m, 2H), 4.51 (d, J=5.6 Hz, 2H), 4.12-4.08 (m, 2H), 4.07-3.97 (m, 3H), 3.83-3.68 (m, 3H), 3.20 (d, J=2.4, 5.6 Hz, 1H), 1.94-1.87 (m, 2H), 1.07-1.02 (m, 2H), 0.79-0.74 (m, 2H).

### Example 385: 7-(2-((6-((1R,4R)-2,5-diazabicyclo[2.2.1]heptan-2-yl)-2-cyclopropylpyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

### Step 1: tert-butyl (1R,4R)-5-(6-cyclopropyl-5-nitropyridin-2-yl)-2,5-diazabicyclo[2.2.1]heptane-2-carboxylate

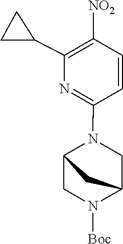 [SMILES: CC(C)(C)OC(=O)N1C[C@H]2C[C@@H]1CN2c1ccc([N+](=O)[O-])c(C2CC2)n1]

A solution of 6-chloro-2-cyclopropyl-3-nitro-pyridine (25 mmol), tert-butyl (1R,4R)-2,5-diazabicyclo[2.2.1]heptane-2-carboxylate (25 mmol) and N,N′-diisopropylethylamine (50 mmol) in acetonitrile (100 mL) was stirred at 80° C. for 12 hours. The reaction mixture was cooled down to room temperature and concentrated under reduced pressure to afford a residue that was purified by silica gel chromatography (30% ethyl acetate in hexanes). The title compound was isolated in 77% yield. MS (ESI) m/z: 361.2 [M+H]⁺. ¹H NMR (400 MHz, CDCl₃) δ 8.15 (d, J=9.2 Hz, 1H), 6.08 (d, J=9.2 Hz, 1H), 4.95-4.87 (m, 1H), 4.70-4.56 (m, 1H), 3.54-3.30 (m, 4H), 3.17-3.11 (m, 1H), 1.97-1.94 (m, 2H), 1.44 (s, 9H), 1.20-1.17 (m, 2H), 1.05 (dd, J=8.0, 3.2 Hz, 2H).

### Step 2: tert-butyl (1R,4R)-5-(5-amino-6-cyclopropylpyridin-2-yl)-2,5-diazabicyclo[2.2.1]heptane-2-carboxylate

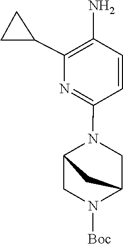 [SMILES: CC(C)(C)OC(=O)N1C[C@H]2C[C@@H]1CN2c1ccc(N)c(C2CC2)n1]

A solution of tert-butyl (1R,4R)-5-(6-cyclopropyl-5-nitro-2-pyridyl)-2,5-diazabicyclo[2.2.1]heptane-2-carboxylate (19.4 mmol), iron powder (97.1 mmol) and ammonia hydrochloride (97.1 mmol) in ethanol (80 mL) and water (20 mL) was stirred at 80° C. for 5 hours. The reaction mixture was cooled down to room temperature, filtered and concentrated. The residue was purified by silica gel chromatography (50% ethyl acetate in hexanes) to afford the title compound in 99% yield. MS (ESI) m/z: 331.1 [M+H]⁺

### Step 3: tert-butyl (1R,4R)-5-(5-((4-chloro-5-(trifluoromethyl)pyrimidin-2-yl)amino)-6-cyclopropylpyridin-2-yl)-2,5-diazabicyclo[2.2.1]heptane-2-carboxylate

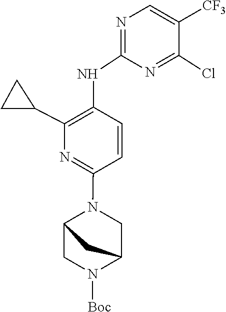 [SMILES: CC(C)(C)OC(=O)N1C[C@H]2C[C@@H]1CN2c1ccc(Nc2ncc(C(F)(F)F)c(Cl)n2)c(C2CC2)n1]

To a solution of tert-butyl (1R,4R)-5-(5-amino-6-cyclopropyl-2-pyridyl)-2,5-diazabicyclo[2.2.1]heptane-2-carboxylate (19.4 mmol) and N,N′-diisopropylethylamine (21.3 mmol) in tert-butanol (65 mL) was added 2,4-dichloro-5-(trifluoromethyl)pyrimidine (21.3 mmol). The mixture was stirred for 1 hour, quenched with water and extracted with ethyl acetate three times. The organic layers were combined and washed with brine, dried over anhydrous sodium sulfate, filtered and concentrated. The crude material was purified by preparative HPLC to afford the title compound in 72% yield. MS (ESI) m/z: 511.3 [M+H]⁺. ¹H NMR (400 MHz, CDCl₃) δ 8.49 (s, 1H), 7.46 (s, 1H), 7.18-7.09 (m, 1H), 6.15 (d, J=8.4 Hz, 1H), 4.85-4.79 (m, 1H), 4.65-4.50 (m, 1H), 3.52-3.32 (m, 4H), 1.99-1.95 (m, 1H), 1.90 (d, J=11.2 Hz, 2H), 1.47-1.44 (m, 9H), 1.12-1.00 (m, 2H), 0.92-0.78 (m, 2H).

### Step 4: N-(6-((1R,4R)-2,5-Diazabicyclo[2.2.1]heptan-2-yl)-2-cyclopropylpyridin-3-yl)-4-chloro-5-(trifluoromethyl)pyrimidin-2-amine

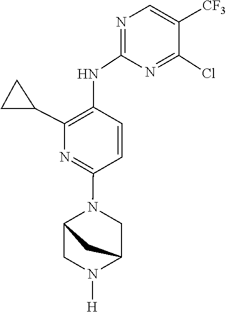 [SMILES: FC(F)(F)c1cnc(Nc2ccc(N3C[C@H]4C[C@@H]3CN4)nc2C2CC2)nc1Cl]

A solution of tert-butyl (1R,4R)-5-[5-[[4-chloro-5-(trifluoromethyl)pyrimidin-2-yl]amino]-6-cyclopropyl-2-pyridyl]-2,5-diazabicyclo[2.2.1]heptane-2-carboxylate (6.85 mmol) in dichloromethane (70 mL) and trifluoroacetic acid (35 mL) was stirred at room temperature for 1 hour. The mixture was concentrated to afford the title compound in 97% yield. MS (ESI) m/z: 411.1 [M+H]⁺.

### Step 5: 1-((1R,4R)-5-(5-((4-chloro-5-(trifluoromethyl)pyrimidin-2-yl)amino)-6-cyclopropylpyridin-2-yl)-2,5-diazabicyclo[2.2.1]heptan-2-yl)-2,2,2-trifluoroethan-1-one

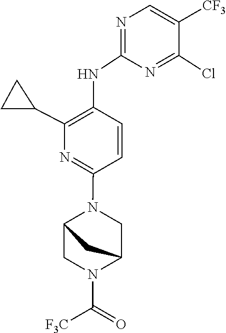 [SMILES: O=C(N1C[C@H]2C[C@@H]1CN2c1ccc(Nc2ncc(C(F)(F)F)c(Cl)n2)c(C2CC2)n1)C(F)(F)F]

To a 0° C. solution of 4-chloro-N-[2-cyclopropyl-6-[(1R,4R)-2,5-diazabicyclo[2.2.1]heptan-2-yl]-3-pyridyl]-5-(trifluoromethyl)pyrimidin-2-amine (6.67 mmol) and triethylamine (20 mmol) in dichloromethane (40 mL) was added (2,2,2-trifluoroacetyl) 2,2,2-trifluoroacetate (8.00 mmol). The mixture was stirred at room temperature for 2 hours, quenched with water and extracted with dichloromethane twice. The organic phase was dried over anhydrous sodium sulfate, filtered and concentrated. The residue was purified by silica gel chromatography (20% ethyl acetate in hexanes) to afford the title compound in 74% yield. MS (ESI) m/z: 507.2 [M+H]⁺.

### Step 6: 1-((1R,4R)-5-(6-cyclopropyl-5-((5-(trifluoromethyl)-4-(trimethylstannyl)pyrimidin-2-yl)amino)pyridin-2-yl)-2,5-diazabicyclo[2.2.1]heptan-2-yl)-2,2,2-trifluoroethan-1-one

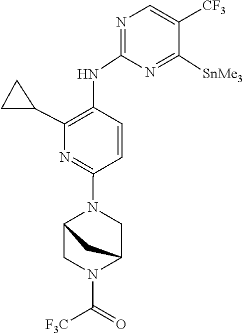

To a solution of 1-[(1R,4R)-5-[5-[[4-chloro-5-(trifluoromethyl)pyrimidin-2-yl]amino]-6-cyclopropyl-2-pyridyl]-2,5-diazabicyclo[2.2.1]heptan-2-yl]-2,2,2-trifluoro-ethanone (2.96 mmol) in dioxane (15 mL) was added 4-diphenylphosphanylbutyl(diphenyl)phosphane (0.59 mmol), diacetoxy palladium (0.59 mmol) and trimethyl(trimethylstannyl)stannane (7.40 mmol). The mixture was stirred at 95° C. for 12 hours, quenched with water and extracted with ethyl acetate three times. The combined organic layers were washed with brine, dried over anhydrous sodium sulfate, filtered, and concentrated under reduced pressure to afford a residue that was purified by flash chromatography (neutral alumina, 8% ethyl acetate in hexanes). The title compound was isolated in 43% yield. MS (ESI) m/z: 637.2 [M+H]⁺.

### Step 7: 7-(2-((2-cyclopropyl-6-((1R,4R)-5-(2,2,2-trifluoroacetyl)-2,5-diazabicyclo[2.2.1]heptan-2-yl)pyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

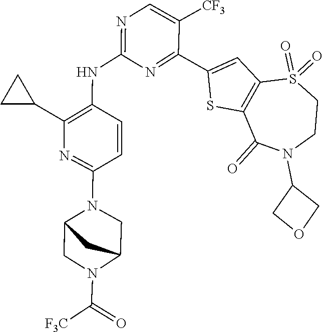 [SMILES: O=C1c2sc(-c3nc(Nc4ccc(N5C[C@H]6C[C@@H]5CN6C(=O)C(F)(F)F)nc4C4CC4)ncc3C(F)(F)F)cc2S(=O)(=O)CCN1C1COC1]

A solution of 1-[(1R,4R)-5-[6-cyclopropyl-5-[[5-(trifluoromethyl)-4-trimethylstannyl-pyrimidin-2-yl]amino]-2-pyridyl]-2,5-diazabicyclo[2.2.1]heptan-2-yl]-2,2,2-trifluoro-ethanone (0.20 mmol), 7-iodo-4-(oxetan-3-yl)-1,1-dioxo-2,3-dihydrothieno[2,3-f][1,4]thiazepin-5-one (0.20 mmol), cuprous iodide (0.20 mmol) and tetrakis(triphenylphosphine) palladium(0) (0.020 mmol) in dioxane (4 mL) was stirred at 80° C. for 12 hours. The mixture was cooled down to room temperature, filtered and concentrated. The residue was purified by preparative TLC (60% ethyl acetate in hexanes) to afford the title compound in 58% yield. MS (ESI) m/z: 744.2 [M+H]⁺.

### Step 8: 7-(2-((6-((1R,4R)-2,5-diazabicyclo[2.2.1]heptan-2-yl)-2-cyclopropylpyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

A solution of 7-[2-[[2-cyclopropyl-6-[(1R,4R)-5-(2,2,2-trifluoroacetyl)-2,5-diazabicyclo[2.2.1]heptan-2-yl]-3-pyridyl]amino]-5-(trifluoromethyl)pyrimidin-4-yl]-4-(oxetan-3-yl)-1,1-dioxo-2,3-dihydrothieno[2,3-f][1,4]thiazepin-5-one (0.11 mmol) and potassium carbonate (1.15 mmol) in acetonitrile (1 mL), ethanol (1 mL), and water (1 mL) was stirred for 12 hours. The mixture was purified by preparative TLC (10% methanol in dichloromethane) to afford the title compound in 40% yield. MS (ESI) m/z: 648.3 [M+H]⁺. ¹H NMR (400 MHz, CDCl₃) δ 8.68 (s, 1H), 8.06 (s, 1H), 7.55-7.52 (m, 1H), 7.20-7.05 (m, 1H), 6.23-6.21 (m, 1H), 5.54-5.53 (m, 1H), 5.05-5.01 (m, 2H), 4.80-4.68 (m, 2H), 4.10-4.09 (m, 3H), 3.81-3.72 (m, 2H), 3.65-3.61 (m, 1H), 3.39-3.37 (m, 1H), 3.20-3.19 (m, 2H), 2.02-2.01 (m, 3H), 1.09-1.05 (m, 2H), 0.89-0.86 (m, 2H).

### Example 386: 7-(2-((6-((1R,4R)-2,5-diazabicyclo[2.2.1]heptan-2-yl)-4-cyclopropylpyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 374, where tert-butyl (R)-2-methylpiperazine-1-carboxylate was replaced with tert-butyl (1R,4R)-2,5-diazabicyclo[2.2.1]heptane-2-carboxylate. The title compound was isolated. MS (ESI) m/z: 648.2 [M+H]⁺. ¹H NMR (400 MHz, CDCl₃) δ ppm 0.68-0.79 (m, 2H) 0.94-1.05 (m, 2H) 1.86 (brd, J=6.0 Hz, 3H) 3.14 (s, 2H) 3.29 (brd, J=9.6 Hz, 1H) 3.62 (br d, J=9.2 Hz, 1H) 3.77 (br s, 2H) 3.96 (br s, 1H) 4.11 (br d, J=1.2 Hz, 2H) 4.66-4.83 (m, 3H) 5.02 (td, J=7.2, 1.6 Hz, 2H) 5.43-5.64 (m, 1H) 5.93 (s, 1H) 7.14-7.25 (m, 1H) 8.06 (br s, 1H) 8.27 (s, 1H) 8.68 (s, 1H).

### Example 387: 7-(2-((4-((1R,4R)-2,5-diazabicyclo[2.2.1]heptan-2-yl)-2-cyclopropylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 310, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (1R,4R)-2,5-diazabicyclo[2.2.1]heptane-2-carboxylate. The title compound was isolated. MS (ESI) m/z: 647.3[M+H]⁺. ¹H NMR (400 MHz, CDCl₃): δ 8.68 (s, 1H), 8.08 (s, 1H), 7.86-7.45 (m, 2H), 6.57-6.48 (m, 1H), 6.37-6.19 (m, 1H), 5.63-5.43 (m, 1H), 5.03 (t, J=7.6 Hz, 2H), 4.73 (t, J=6.8 Hz, 2H), 4.31 (s, 1H), 4.16-4.07 (m, 2H), 3.85-3.72 (m, 3H), 3.69-3.61 (m, 1H), 3.16-3.09 (m, 1H), 3.08-3.01 (m, 1H), 2.97 (d, J=8.8 Hz, 1H), 1.89-1.82 (m, 1H), 1.36-1.24 (m, 2H), 1.02-0.93 (m, 2H), 0.74-0.61 (m, 2H).

### Example 388: (R)-7-(2-((2-cyclopropyl-4-(3-(hydroxymethyl)-4-methylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (R)-7-(2-((2-cyclopropyl-4-(3-(hydroxymethyl)piperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 31% yield. MS (ESI) m/z: 679.2 [M+H]⁺. ¹H NMR (400 MHz, CDCl₃) δ ppm 8.73 (s, 1H) 8.10 (s, 1H) 7.94 (d, J=8.8 Hz, 1H) 7.71 (s, 1H) 6.89-6.98 (m, 1H) 6.78 (br d, J=4.0 Hz, 1H) 5.45-5.60 (m, 1H) 5.03 (t, J=7.2 Hz, 2H) 4.70-4.80 (m, 2H) 4.19-4.28 (m, 1H) 4.09-4.15 (m, 2H) 4.01 (br dd,J=12.4, 1.6 Hz, 1H) 3.72-3.85 (m, 3H) 3.57-3.69 (m, 4H) 3.18-3.26 (m, 1H) 3.01 (s, 3H) 1.83-1.91 (m, 1H) 1.01-1.11 (m, 2H) 0.65-0.75 (m, 2H).

### Example 389: (R)-7-(2-((4-(3,4-dimethylpiperazin-1-yl)-2-ethylphenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (R)-7-(2-((2-ethyl-4-(3-methylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 20% yield. MS (ESI) m/z: 651.2 [M+H]f. ¹H NMR (400 MHz, CDCl₃): δ 8.67 (s, 1H), 8.07 (s, 1H), 7.60-7.39 (m, 1H), 7.11-6.98 (m, 1H), 6.88-6.80 (m, 2H), 5.56-5.52 (m, 1H), 5.03 (t, J=7.6 Hz, 2H), 4.73 (t, J=6.8 Hz, 2H), 4.21-4.07 (m, 2H), 3.78-3.70 (m, 2H), 3.61-3.46 (m, 2H), 3.08-2.89 (m, 2H), 2.63 (q, J=7.6 Hz, 2H), 2.60-2.55 (m, 1H), 2.53-2.44 (m, 1H), 2.42-2.31 (m, 4H), 1.27-1.23 (m, 3H), 1.22-1.18 (m, 3H).

### Example 390: (S)-4-(3-cyclopropyl-4-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)phenyl)-N,1-dimethylpiperazine-2-carboxamide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (S)-4-(3-cyclopropyl-4-((4-(4-(oxetan-3-yl)-1,1-dioxido-5-oxo-2,3,4,5-tetrahydrothieno[2,3-f][1,4]thiazepin-7-yl)-5-(trifluoromethyl)pyrimidin-2-yl)amino)phenyl)-N-methylpiperazine-2-carboxamide. The title compound was isolated in 37% yield. MS (ESI) m/z: 706.3 [M+H]⁺. ¹H NMR (400 MHz, DMSO-d₆) δ 9.77 (s, 1H), 8.82-8.70 (m, 1H), 7.88-7.80 (m, 2H), 7.19-7.10 (m, 1H), 6.75 (d, J=2.4, 8.8 Hz, 1H), 6.45 (s, 1H), 5.21 (s, 1H), 4.74-4.68 (m, 4H), 4.00 (s, 4H), 3.54 (s, 1H), 2.88 (br d, J=11.2 Hz, 1H), 2.74-2.65 (m, 3H), 2.61 (d, J=4.8 Hz, 3H), 2.47-2.45 (m, 2H), 2.14 (s, 3H), 1.92-1.88 (m, 1H), 0.80-0.77 (m, 2H), 0.62-0.59 (m, 2H).

### Example 391: (R)-7-(2-((4-cyclopropyl-6-(3,4-dimethylpiperazin-1-yl)pyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (R)-7-(2-((4-cyclopropyl-6-(3-methylpiperazin-1-yl)pyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 91% yield. MS (ESI) m/z: 664.2 [M+H]⁺. ¹H NMR (400 MHz, CDCl₃-d) δ, 0.70-0.80 (m, 2H) 0.99-1.08 (m, 2H) 1.22 (br d, J=5.2 Hz, 3H) 1.82-1.92 (m, 1H) 2.16-2.54 (m, 5H) 2.68-2.87 (m, 1H) 2.91-3.05 (m, 1H) 3.08-3.27 (m, 1H) 3.78 (br d, J=5.6 Hz, 2H) 4.01-4.19 (m, 4H) 4.73 (t, J=6.8 Hz, 2H) 5.02 (t, J=7.6 Hz, 2H) 5.54 (m, J=6.4 Hz, 1H) 6.28 (s, 1H) 7.20 (br s, 1H) 8.06 (s, 1H) 8.41 (br s, 1H) 8.69 (s, 1H).

### Example 392: 7-(2-((2-cyclopropyl-4-(6-methyl-2,6-diazaspiro[3.3]heptan-2-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((2-cyclopropyl-4-(2,6-diazaspiro[3.3]heptan-2-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 29% yield. MS (ESI) m/z: 661.4 [M+H]⁺. ¹H NMR (400 MHz, CDCl₃) δ ppm 0.63-0.71 (m, 2H) 0.94-1.02 (m, 2H) 1.85-1.90 (m, 1H) 2.37 (s, 3H) 3.45 (s, 4H) 3.77 (brt, J=6.0 Hz, 2H) 3.95 (s, 4H) 4.06-4.14 (m, 2H) 4.74 (t, J=6.8 Hz, 2H) 5.03 (t, J=7.6 Hz, 2H) 5.54 (quin, J=6.8 Hz, 1H) 6.19 (br s, 1H) 6.39 (br d, J=8.4 Hz, 1H) 7.36-7.80 (m, 2H) 8.07 (s, 1H) 8.68 (s, 1H).

### Example 393: 7-(2-((2-cyclopropyl-4-((1R,4R)-5-methyl-2,5-diazabicyclo[2.2.1]heptan-2-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((4-((1R,4R)-2,5-diazabicyclo[2.2.1]heptan-2-yl)-2-cyclopropylphenyl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 54% yield. MS (ESI) m/z: 619.2[M+H]⁺. ¹H NMR (400 MHz, CDCl₃) δ 8.68 (s, 1H), 8.06 (s, 1H), 7.83-7.70 (m, 1H), 7.64-7.49 (m, 1H), 6.58-6.46 (m, 1H), 6.37-6.29 (m, 1H), 4.37-4.26 (m, 1H), 3.97-3.87 (m, 2H), 3.79-3.70 (m, 3H), 3.54-3.42 (m, 2H), 3.25 (s, 3H), 3.20-3.09 (m, 1H), 2.98-2.78 (m, 1H), 2.60-2.48 (m, 3H), 2.24-2.14 (m, 1H), 2.08-2.02 (m, 1H), 1.90-1.86 (m, 1H), 1.04-0.99 (m, 2H), 0.72-0.65 (m, 2H).

### Example 394: 7-(2-((2-cyclopropyl-4-(6-methyl-3,6-diazabicyclo[3.1.1]heptan-3-yl)phenyl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((4-(3,6-diazabicyclo[3.1.1]heptan-3-yl)-2-cyclopropylphenyl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 32% yield. MS (ESI) m/z: 619.3 [M+H]⁺. ¹H NMR (400 MHz, CDCl₃) δ 8.68 (s, 1H), 8.06 (s, 1H), 7.92-7.38 (m, 2H), 6.74-6.65 (m, 1H), 6.52 (s, 1H), 3.95-3.86 (m, 2H), 3.79-3.68 (m, 4H), 3.62-3.55 (m, 2H), 3.42-3.31 (m, 2H), 3.25 (s, 3H), 2.72-2.60 (m, 1H), 2.18 (s, 3H), 1.96-1.89 (m, 1H), 1.66-1.62 (m, 1H), 1.06-0.95 (m, 2H), 0.79-0.70 (m, 2H).

### Example 395: 7-(2-((6-((1R,4R)-2,5-Diazabicyclo[2.2.1]heptan-2-yl)-2-cyclopropylpyridin-3-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 310, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (1R,4R)-5-(5-((4-chloro-5-(trifluoromethyl)pyrimidin-2-yl)amino)-6-cyclopropylpyridin-2-yl)-2,5-diazabicyclo[2.2.1]heptane-2-carboxylate in step 1 and 7-iodo-4-(oxetan-3-yl)-3,4-dihydrothieno[2,3-f][1,4]thiazepin-5(2H)-one 1,1-dioxide was replaced with 7-bromo-4-methyl-3,4-dihydrothieno[2,3-f][1,4]thiazepin-5(2H)-one 1,1-dioxide in step 4. The title compound was isolated. MS (ESI) m/z: 606.2 [M+H]⁺. ¹H NMR (400 MHz, CDCl₃) δ 8.68 (s, 1H), 8.03 (s, 1H), 7.52 (d, J=8.4 Hz, 1H), 7.19 (s, 1H), 6.26-6.22 (m, 1H), 4.88 (s, 1H), 4.38-4.34 (m, 1H), 3.93-3.91 (m, 2H), 3.73-3.59 (m, 4H), 3.57-3.30 (m, 1H), 3.30-3.28 (m, 1H), 3.24 (s, 3H), 2.17 (s, 1H), 2.06-2.02 (m, 2H), 1.09-1.01 (m, 2H), 0.90-0.87s (m, 2H).

### Example 396: 7-(2-((2-Cyclopropyl-6-((1R,4R)-5-methyl-2,5-diazabicyclo[2.2.1]heptan-2-yl)pyridin-3-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((6-((1R,4R)-2,5-diazabicyclo[2.2.1]heptan-2-yl)-2-cyclopropylpyridin-3-yl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 38% yield. MS (ESI) m/z: 620.1 [M+H]⁺. ¹H NMR (400 MHz, CDCl₃) δ 8.67 (s, 1H), 8.04 (s, 1H), 7.60-7.53 (m, 1H), 7.16-7.13 (m, 1H), 6.22-6.21 (m, 1H), 4.70 (s, 1H), 3.91-3.87 (m, 2H), 3.72-3.63 (m, 4H), 3.40-3.32 (m, 1H), 3.23 (s, 3H), 3.06-3.05 (m, 1H), 2.88-2.66 (m, 1H), 2.51 (s, 3H), 2.03-2.02 (m, 1H), 1.97-1.88 (m, 2H), 1.11-1.07 (m, 2H), 0.89-0.86 (m, 2H).

### Example 397: 7-(2-((6-(3,6-diazabicyclo[3.1.1]heptan-3-yl)-2-cyclopropylpyridin-3-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 375, steps 2-4, where tert-butyl (R)-2-methylpiperazine-1-carboxylate was replaced with tert-butyl 3,6-diazabicyclo[3.1.1]heptane-6-carboxylate and Example 308, steps 4-6, where tert-butyl (R)-4-(4-((4-chloro-5-(trifluoromethyl)pyrimidin-2-yl)amino)-3-cyclopropylphenyl)-2-methylpiperazine-1-carboxylate was replaced with tert-butyl 3-(5-((4-chloro-5-(trifluoromethyl)pyrimidin-2-yl)amino)-6-cyclopropylpyridin-2-yl)-3,6-diazabicyclo[3.1.1]heptane-6-carboxylate. The title compound was isolated. MS (ESI) m/z: 606.3[M+H]⁺. ¹H NMR (400 MHz, methanol-d₄) δ 8.72-8.58 (m, 1H), 8.00-7.96 (m, 1H), 7.52-7.46 (m, 1H), 6.49-6.47 (m, 1H), 4.18-4.08 (m, 2H), 3.95-3.78 (m, 8H), 3.16 (s, 3H), 2.89-2.83 (m, 1H), 2.15-2.09 (m, 1H), 1.82-1.79 (m, 1H), 1.13-1.04 (m, 2H), 0.83 (dd, J=7.6, 2.8 Hz, 2H).

### Example 398: 7-(2-((2-cyclopropyl-6-(6-methyl-3,6-diazabicyclo[3.1.1]heptan-3-yl)pyridin-3-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((6-(3,6-diazabicyclo[3.1.1]heptan-3-yl)-2-cyclopropylpyridin-3-yl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 33% yield. MS (ESI) m/z: 620.3[M+H]⁺. ¹H NMR (400 MHz, methanol) δ 8.73-8.55 (m, 1H), 8.00-7.95 (m, 1H), 7.54-7.42 (m, 1H), 6.46 (s, 1H), 3.97-3.60 (m, 10H), 3.20-3.15 (m, 3H), 2.67-2.61 (m, 1H), 2.31-2.23 (m, 4H), 1.75-1.66 (m, 1H), 1.14-1.05 (m, 2H), 0.84-0.82 (m 2H).

### Example 399: 7-(2-((6-(3,6-Diazabicyclo[3.1.1]heptan-3-yl)-2-cyclopropylpyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 375, where tert-butyl (R)-2-methylpiperazine-1-carboxylate was replaced with tert-butyl 3,6-diazabicyclo[3.1.1]heptane-6-carboxylate in step 2. The title compound was isolated. MS (ESI) m/z: 648.1[M+H]⁺. ¹H NMR (400 MHz, methanol-d₄) δ 8.72-8.55 (m, 1H), 8.01-7.97 (m, 1H), 7.50-7.44 (m, 1H), 6.44 (d, J=8.8 Hz, 1H), 5.30-5.25 (m, 1H), 4.07-3.97 (m, 4H), 3.92-3.85 (m, 2H), 3.82-3.72 (m, 4H), 2.82-2.77 (m, 1H), 2.14-2.08 (m, 1H), 1.73-1.71 (m, 1H), 1.15-1.02 (m, 2H), 0.82 (dd, J=7.6, 2.8 Hz, 2H).

### Example 400: 7-(2-((2-Cyclopropyl-6-(6-methyl-3,6-diazabicyclo[3.1.1]heptan-3-yl)pyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((6-(3,6-Diazabicyclo[3.1.1]heptan-3-yl)-2-cyclopropylpyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 30% yield. MS (ESI) m/z: 662.3[M+H]⁺. ¹H NMR (400 MHz, CDCl₃) δ 8.69 (s, 1H), 8.07 (s, 1H), 7.68-7.53 (m, 1H), 7.24-7.15 (m, 1H), 6.38 (d, J=8.0 Hz, 1H), 5.56-5.50 (m, 1H), 5.08-4.97 (m, 2H), 4.73 (t, J=6.4 Hz, 2H), 4.13-4.08 (m, 2H), 3.90-3.72 (m, 6H), 3.62-3.57 (m, 2H), 2.78-2.71 (m, 1H), 2.26 (s, 3H), 2.06-2.05 (m, 1H), 1.76-1.62 (m, 1H), 1.17-1.14 (m, 2H), 0.92-0.88 (m, 2H).

### Example 401: 7-(2-((4-cyclopropyl-6-((1R,4R)-5-methyl-2,5-diazabicyclo[2.2.1]heptan-2-yl)pyridin-3-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((6-((1R,4R)-2,5-diazabicyclo[2.2.1]heptan-2-yl)-4-cyclopropylpyridin-3-yl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 11% yield. MS (ESI).m/z: 665.2 [M+H]⁺. ¹H NMR (400 MHz, CDCl₃) δ ppm 8.68 (s, 1H) 8.27 (s, 1H) 8.04 (br s, 1H) 7.18 (s, 1H) 5.97 (br s, 1H) 4.68-4.86 (m, 1H) 3.66-3.93 (m, 6H) 3.39-3.50 (m, 1H) 3.07-3.28 (m, 5H) 2.51-2.70 (m, 3H) 2.10-2.18 (m, 1H) 1.84-1.87 (m, 2H) 0.96-1.06 (m, 2H) 0.71-0.78 (m, 2H).

### Example 402: 7-(2-((4-cyclopropyl-6-((1R,4R)-5-methyl-2,5-diazabicyclo[2.2.1]heptan-2-yl)pyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((6-((1R,4R)-2,5-diazabicyclo[2.2.1]heptan-2-yl)-4-cyclopropylpyridin-3-yl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated in 68% yield. MS (ESI) m/z: 662.1 [M+H]⁺

¹H NMR (400 MHz, CDCl₃) δ ppm 0.67-0.78 (m, 2H) 1.00 (br dd, J=6.0, 2.4 Hz, 2H) 1.85 (ddd, J=13.6, 8.0, 5.2 Hz, 2H) 2.07 (br d, J=10.2 Hz, 1H) 2.51 (br s, 3H) 2.68-2.94 (m, 1H) 2.96-3.13 (m, 1H) 3.33-3.47 (m, 1H) 3.63 (br d, J=14.4 Hz, 2H) 3.76 (br s, 2H) 4.02-4.19 (m, 2H) 4.67-4.80 (m, 3H) 5.02 (br t, J=7.6 Hz, 2H) 5.44-5.64 (m, 1H) 5.95 (s, 1H) 7.14 (br s, 1H) 8.06 (br s, 1H) 8.27 (s, 1H) 8.68 (s, 1H).

### Example 403: 7-(2-((4-cyclopropyl-6-(6-methyl-3,6-diazabicyclo[3.1.1]heptan-3-yl)pyridin-3-yl)amino)-5-(trifluoromethyl)pyrimidin-4-yl)-4-methyl-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with 7-(2-((6-(3,6-diazabicyclo[3.1.1]heptan-3-yl)-4-cyclopropylpyridin-3-yl)amino)-5-(trifluoromethyl)pyrimidin-4-yl)-4-methyl-3,4-dihydrothieno[2,3-f][1,4]thiazepin-5(2H)-one 1,1-dioxide. The title compound was isolated in 39% yield. MS (ESI) m/z: 620.2 [M+H]⁺. ¹H NMR (400 MHz, CDCl₃) δ 8.69 (s, 1H), 8.40 (d, J=1.6 Hz, 1H), 8.06 (s, 1H), 7.20 (s, 1H), 6.16 (s, 1H), 4.05-3.93 (m, 2H), 3.91 (s, 2H), 3.83-3.64 (m, 6H), 3.24 (s, 3H), 2.97-2.82 (m, 1H), 2.34 (s, 3H), 2.10-2.05 (m, 1H), 1.96-1.93 (m, 1H), 1.08-1.02 (m, 2H), 0.79 (d, J=5.4 Hz, 2H).

### Example 404: (R)—N-(2-cyclopropyl-4-(3-methylpiperazin-1-yl)phenyl)-4-(4-(methylsulfonyl)thiophen-2-yl)-5-(trifluoromethyl)pyrimidin-2-amine

The title compound was prepared analogously to Example 308, where 7-bromo-4-cyclopropyl-3,4-dihydrothieno[2,3-f][1,4]thiazepin-5(2H)-one 1,1-dioxide was replaced with 2-bromo-4-(methylsulfonyl)thiophene in step 5. The title compound was isolated. MS (ESI) m/z: 538.2 [M+1H]⁺. ¹H NMR (400 MHz, methanol-d₄) δ=8.75-8.57 (m, 1H), 8.46 (s, 1H), 8.00 (s, 1H), 7.51-7.33 (m, 1H), 6.87 (dd, J=8.8, 2.8 Hz, 1H), 6.67 (d, J=2.4 Hz, 1H), 3.55 (t, J=10.4 Hz, 2H), 3.18 (s, 3H), 3.12 (d, J=2.4 Hz, 1H), 3.05-2.97 (m, 2H), 2.71-2.64 (m, 1H), 2.43-2.34 (m, 1H), 2.00-1.92 (m, 1H), 1.19 (d, J=6.4 Hz, 3H), 0.93-0.86 (m, 2H), 0.69-0.60 (m, 2H).

### Example 405: N-(4-(3,6-diazabicyclo[3.1.1]heptan-3-yl)-2-cyclopropylphenyl)-4-(4-(methylsulfonyl)thiophen-2-yl)-5-(trifluoromethyl)pyrimidin-2-amine

The title compound was prepared analogously to Example 308, where with tert-butyl (R)-4-(3-cyclopropyl-4-((5-(trifluoromethyl)-4-(trimethylstannyl)pyrimidin-2-yl)amino)phenyl)-2-methylpiperazine-1-carboxylate and 7-bromo-4-cyclopropyl-3,4-dihydrothieno[2,3-f][1,4]thiazepin-5(2H)-one 1,1-dioxide were replaced with tert-butyl 3-[3-cyclopropyl-4-[[5-(trifluoromethyl)-4-trimethylstannyl-pyrimidin-2-yl]amino]phenyl]-3,6-diazabicyclo[3.1.1]heptane-6-carboxylate and 2-bromo-4-methylsulfonyl-thiophene. The title compound was isolated. MS (ESI) m/z: 536.2[M+H]⁺. ¹H NMR (400 MHz, methanol-d₄) δ 8.66-8.58 (m, 1H), 8.43-8.41 (m, 1H), 7.99 (s, 1H), 7.42-7.24 (m, 1H), 6.65 (d, J=8.4 Hz, 1H), 6.42 (s, 1H), 4.02-3.81 (m, 2H), 3.62-3.56 (m, 4H), 3.17 (s, 3H), 2.80-2.68 (m, 1H), 2.00-1.93 (m, 1H), 1.71 (d, J=9.2 Hz, 1H), 0.90-0.85 (m, 2H), 0.68-0.64 (m, 2H).

### Example 406: (R)-7-(2-((2-ethyl-4-(3-methylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 319, steps 1-4, where tert-butyl (1S,4S)-2,5-diazabicyclo[2.2.1]heptane-2-carboxylate was replaced with tert-butyl (R)-2-methylpiperazine-1-carboxylate and Example 310, where tert-butyl (1S,4S)-5-(4-((4-chloro-5-(trifluoromethyl)pyrimidin-2-yl)amino)-3-cyclopropylphenyl)-2,5-diazabicyclo[2.2.1]heptane-2-carboxylate was replaced with tert-butyl (R)-4-(4-((4-chloro-5-(trifluoromethyl)pyrimidin-2-yl)amino)-3-ethylphenyl)-2-methylpiperazine-1-carboxylate in step 1. The title compound was isolated. MS (ESI) m/z: 637.3 [M+H]⁺. ¹H NMR (400 MHz, CDCl₃): δ 8.67 (s, 1H), 8.07 (s, 1H), 7.56-7.42 (m, 1H), 7.08-6.98 (m, 1H), 6.84 (br s, 2H), 5.61-5.48 (m, 1H), 5.03 (t, J=7.6 Hz, 2H), 4.73 (t, J=6.8 Hz, 2H), 4.10 (br d, J=3.6 Hz, 2H), 3.77 (br t, J=5.4 Hz, 2H), 3.57 (br d, J=11.6 Hz, 2H), 3.22-3.14 (m, 1H), 3.12-3.01 (m, 2H), 2.81 (dt, J=11.4, 3.2, Hz, 1H), 2.64 (q, J=7.6 Hz, 2H), 2.46 (br t, J=10.8 Hz, 1H), 1.25-1.19 (m, 6H).

### Example 407: (R)-4-cyclopropyl-7-(2-((2-ethyl-4-(3-(hydroxymethyl)piperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 355, where 7-iodo-4-(oxetan-3-yl)-3,4-dihydrothieno[2,3-f][1,4]thiazepin-5(2H)-one 1,1-dioxide was replaced with 4-cyclopropyl-7-iodo-3,4-dihydrothieno[2,3-f][1,4]thiazepin-5(2H)-one 1,1-dioxide. The title compound was isolated. MS (ESI) m/z: 637.4 [M+H]⁺. ¹H NMR (400 MHz, CDCl₃) δ 8.67 (s, 1H), 8.03 (s, 1H), 7.56-7.41 (m, 1H), 7.08 (br s, 1H), 6.91-6.79 (m, 2H), 3.97-3.87 (m, 3H), 3.79 (br dd, J=6.8, 11.2 Hz, 1H), 3.63 (br t, J=5.6 Hz, 4H), 3.38 (br d, J=12.0 Hz, 1H), 3.32-3.26 (m, 1H), 3.21-3.12 (m, 2H), 2.94-2.89 (m, 2H), 2.64 (q, J=7.6 Hz, 2H), 1.23 (t, J=7.6 Hz, 3H), 0.98-0.90 (m, 4H).

### Example 408: (R)-4-cyclopropyl-7-(2-((2-ethyl-4-(3-(hydroxymethyl)-4-methylpiperazin-1-yl)phenyl)amino)-5-(trifluoromethyl)pyrimidin-4-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (R)-4-cyclopropyl-7-(2-((2-ethyl-4-(3-(hydroxymethyl)piperazin-1-yl)phenyl)amino)-5-(trifluoromethyl)pyrimidin-4-yl)-3,4-dihydrothieno[2,3-f][1,4]thiazepin-5(2H)-one 1,1-dioxide. MS (ESI) m/z: 651.5 [M+H]⁺. ¹H NMR (400 MHz, CDCl₃) δ 8.66 (s, 1H), 8.02 (s, 1H), 7.59-7.34 (m, 1H), 7.18-7.03 (m, 1H), 6.93-6.80 (m, 2H), 4.01 (br dd, J=4.0, 12.0 Hz, 1H), 3.93 (br s, 2H), 3.77-3.67 (m, 1H), 3.67-3.52 (m, 4H), 3.52-3.46 (m, 1H), 3.23-2.97 (m, 3H), 2.90 (br s, 1H), 2.81-2.69 (m, 1H), 2.66-2.61 (m, 2H), 2.56 (br s, 3H), 2.10-2.06 (m, 1H), 1.23 (br t, J=7.6 Hz, 3H), 0.97-0.90 (m, 4H).

### Example 409: (R)-7-(2-((2-Ethyl-4-(3-(hydroxymethyl)-4-methylpiperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide

The title compound was prepared analogously to Example 306, where 7-(2-((4-((1S,4S)-2,5-diazabicyclo[2.2.1]heptan-2-yl)-2-cyclopropylphenyl)amino)-5-(trifluoromethyl)pyrimidin-4-yl)-4-cyclopropyl-3,4-dihydrothieno[2,3-f][1,4]thiazepin-5(2H)-one 1,1-dioxide was replaced with (R)-7-(2-((2-ethyl-4-(3-(hydroxymethyl)piperazin-1-yl)phenyl)amino)-5-(trifluoromethyl)pyrimidin-4-yl)-4-(oxetan-3-yl)-3,4-dihydrothieno[2,3-f][1,4]thiazepin-5(2H)-one 1,1-dioxide. The title compound was isolated. MS (ESI) m/z: 667.3 [M+H]⁺. ¹H NMR (400 MHz, CDCl₃) δ 8.68 (s, 1H), 8.07 (s, 1H), 7.58-7.37 (m, 1H), 7.07 (br s, 1H), 6.93-6.77 (m, 2H), 5.59-5.43 (m, 1H), 5.02 (t, J=7.6 Hz, 2H), 4.73 (t, J=6.8 Hz, 2H), 4.10 (br s, 2H), 4.01 (dd, J=4.0, 12.0 Hz, 1H), 3.76 (m, 2H), 3.69 (br d, J=12.0 Hz, 1H), 3.57 (br t, J=12.0 Hz, 2H), 3.19-2.97 (m, 3H), 2.77-2.65 (m, 2H), 2.65-2.58 (m, 2H), 2.54 (s, 3H), 1.23 (t, J=7.6 Hz, 3H).

### Example A: Biochemical Characterization of ULK1 Inhibitors

### Materials:

Recombinant human ULK1 (1-649) protein expressed in Sf9 insect cells was obtained from SignalChem (Vancouver, BC), catalog #U01-11G. Active kinase was purified via N-terminal GST tag to >85% purity.

ADP GLO Kinase Assay Reagents were obtained from Promega Corp (Madison, WI), catalog #V9104.

MBP (myelin basic protein) substrate, bovine, purified, was obtained from Sigma (St. Louis, MO), catalog #M1891.

### Methods:

### ADP Glo Kinase Activity Assay

ULK1 inhibitor potency was determined using the luminescent ADP Glo Kinase Assay (Promega), a universal ADP detection system that measures kinase activity by quantifying the amount of ADP produced during a kinase reaction. ULK1 kinase reactions are performed in a standard 96-well microplate (20 uL reaction volume) at 25° C. for 40 minutes in the presence or absence of the test compound (12-point dilution series starting at 10 uM). Addition of MBP substrate and ATP initiates the kinase reaction.

The standard ADP Glo assay is performed in two steps. In step one, following the kinase reaction, an equal volume of ADP-Glo™ Reagent (20 uL) is added to terminate the kinase reaction and deplete the remaining ATP. In the second step, the Kinase Detection Reagent (10 uL) is added to simultaneously convert ADP to ATP and allow the newly synthesized ATP to be measured using a luciferase/luciferin reaction. Step two of the ADP Glo assay is performed in a white 384-well plate (20 uL final volume). Thirty minutes after the addition of the Detection reagent, light generated from the luciferase reaction is measured (1 second exposure) using a microplate compatible luminometer (CLARIOstar, BMG Labtech).

Inhibition of ULK1 kinase activity is determined for each test compound by comparison to DMSO only control wells. Inhibitor IC50 was calculated by plotting the change in luminescence after 30 minutes (response) versus inhibitor concentration (in log molar) then applying the four-parameter variable slope curve fit (GraphPad Prism). Inhibition of ULK1 in biochemical assay is found in Table 3.

TABLE 3

| | ULK1 |
|---|---|
| Example # | biochemical IC₅₀ |
| 1 | A |
| 2 | B |
| 3 | A |
| 4 | A |
| 5 | A |
| 6 | C |
| 7 | A |
| 8 | B |
| 9 | A |
| 10 | A |
| 11 | B |
| 12 | A |
| 13 | A |
| 14 | A |
| 15 | B |
| 16 | A |
| 17 | A |
| 18 | B |
| 19 | A |
| 20 | B |
| 21 | A |
| 22 | A |
| 23 | A |
| 24 | B |
| 25 | B |
| 26 | E |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | B |
| 39 | A |
| 40 | A |
| 41 | B |
| 42 | A |
| 43 | A |
| 44 | A |
| 45 | A |
| 46 | A |
| 47 | A |
| 48 | A |
| 49 | A |
| 50 | E |
| 51 | A |
| 52 | A |
| 53 | A |
| 54 | D |
| 55 | A |
| 56 | A |
| 57 | A |
| 58 | D |
| 59 | A |
| 60 | A |
| 61 | D |
| 62 | B |
| 63 | A |
| 64 | A |
| 65 | A |
| 66 | A |
| 67 | B |
| 68 | A |
| 69 | A |
| 70 | B |
| 71 | A |
| 72 | A |
| 73 | A |
| 74 | E |
| 75 | A |
| 76 | B |
| 77 | A |
| 78 | A |
| 79 | D |
| 80 | A |
| 81 | A |
| 82 | A |
| 83 | A |
| 84 | A |
| 85 | A |
| 86 | A |
| 87 | A |
| 88 | A |
| 89 | B |
| 90 | A |
| 91 | A |
| 92 | A |
| 93 | A |
| 94 | A |
| 95 | A |
| 96 | B |
| 97 | A |
| 98 | A |
| 99 | A |
| 100 | A |
| 101 | A |
| 102 | A |
| 103 | A |
| 104 | A |
| 105 | A |
| 106 | A |
| 107 | B |
| 108 | D |
| 109 | A |
| 110 | A |
| 111 | A |
| 112 | A |
| 113 | B |
| 114 | B |
| 115 | A |
| 116 | A |
| 117 | A |
| 118 | B |
| 119 | A |
| 120 | B |
| 121 | C |
| 122 | D |
| 123 | B |
| 124 | D |
| 125 | D |
| 126 | B |
| 127 | A |
| 128 | A |
| 129 | A |
| 130 | A |
| 131 | A |
| 132 | A |
| 133 | A |
| 134 | A |
| 135 | A |

A: IC₅₀ ≤ 10 nM
B: 10 nM < IC₅₀ ≤ 50 nM
C: 50 nM < IC₅₀ ≤ 100 nM
D: 100 nM < IC₅₀ ≤ 500 nM
E: IC₅₀ > 500 nM

### Example B: Biochemical Characterization of ULK1 Inhibitors (New Method)

### Materials:

Recombinant human ULK1 (1-649) protein expressed in Sf9 insect cells was obtained from SignalChem (Vancouver, BC), catalog #U01-11G. Active kinase was purified via N-terminal GST tag to >85% purity.

ADP GLO Kinase Assay Reagents were obtained from Promega Corp (Madison, WI), catalog #V9104.

Synthetic peptide substrate (ULKtide) with the amino acid sequence YANWLAASIYLDGKKK was obtained from ThermoFisher Scientific USA.

### Methods:

### ADP Glo Kinase Activity Assay

ULK1 inhibitor potency was determined using the luminescent ADP Glo Kinase Assay (Promega), a universal ADP detection system that measures kinase activity by quantifying the amount of ADP produced during a kinase reaction. ULK1 kinase reactions are performed in a standard 96-well microplate (20 μL reaction volume) at 25° C. for 40 minutes in the presence or absence of the test compound (12-point dilution series starting at 10 μM). Addition of peptide substrate and ATP initiates the kinase reaction.

The standard ADP Glo assay is performed in two steps. In step one, following the kinase reaction, an equal volume of ADP-Glo™ Reagent (20 μL) is added to terminate the kinase reaction and deplete the remaining ATP. In the second step, the Kinase Detection Reagent (10 μL) is added to simultaneously convert ADP to ATP and allow the newly synthesized ATP to be measured using a luciferase/luciferin reaction. Step two of the ADP Glo assay is performed in a white 384-well plate (20 μL final volume). Thirty minutes after the addition of the Detection reagent, light generated from the luciferase reaction is measured (1 second exposure) using a microplate compatible luminometer (CLARIOstar, BMG Labtech).

Inhibition of ULK1 kinase activity is determined for each test compound by comparison to DMSO only control wells. Inhibitor IC50 was calculated by plotting the change in luminescence after 30 minutes (response) versus inhibitor concentration (in log molar) then applying the four-parameter variable slope curve fit (GraphPad Prism). Inhibition of ULK1 in biochemical assay is found in Table 4.

TABLE 4

| | ULK1 |
|---|---|
| Example # | biochemical IC₅₀ |
| 136 | D |
| 137 | E |
| 138 | A |
| 139 | A |
| 140 | A |
| 141 | A |
| 142 | A |
| 143 | A |
| 144 | A |
| 145 | B |
| 146 | A |
| 147 | A |
| 148 | A |
| 149 | C |
| 150 | C |
| 151 | A |
| 152 | A |
| 153 | A |
| 154 | A |
| 155 | A |
| 156 | A |
| 157 | A |
| 158 | A |
| 159 | A |
| 160 | A |
| 161 | A |
| 162 | A |
| 163 | A |
| 164 | A |
| 165 | E |
| 166 | E |
| 167 | A |
| 168 | A |
| 169 | A |
| 170 | C |
| 171 | A |
| 172 | A |
| 173 | A |
| 174 | A |
| 175 | A |
| 176 | E |
| 177 | A |
| 178 | E |
| 179 | A |
| 180 | A |
| 181 | A |
| 182 | E |
| 183 | E |
| 184 | A |
| 185 | A |
| 186 | A |
| 187 | A |
| 188 | A |
| 189 | D |
| 190 | A |
| 191 | D |
| 192 | A |
| 193 | A |
| 194 | A |
| 195 | A |
| 196 | A |
| 197 | A |
| 198 | A |
| 199 | A |
| 200 | E |
| 201 | A |
| 202 | A |
| 203 | A |
| 204 | A |
| 205 | A |
| 206 | A |
| 207 | A |
| 208 | A |
| 209 | A |
| 210 | A |
| 211 | C |
| 212 | A |
| 213 | A |
| 214 | A |
| 215 | A |
| 216 | A |
| 217 | A |
| 218 | A |
| 219 | A |
| 220 | A |
| 221 | A |
| 222 | A |
| 223 | A |
| 224 | A |
| 225 | A |
| 226 | A |
| 227 | A |
| 228 | A |
| 229 | A |
| 230 | A |
| 231 | A |
| 232 | A |
| 233 | A |
| 234 | A |
| 235 | A |
| 236 | A |
| 237 | A |
| 238 | B |
| 239 | A |
| 240 | A |
| 241 | A |
| 242 | A |
| 243 | A |
| 244 | A |
| 245 | A |
| 246 | A |
| 247 | A |
| 248 | A |
| 249 | A |
| 250 | A |
| 251 | A |
| 252 | A |
| 253 | A |
| 254 | A |
| 255 | A |
| 256 | A |
| 257 | A |
| 258 | A |
| 259 | A |
| 260 | A |
| 261 | A |
| 262 | A |
| 263 | A |
| 264 | A |
| 265 | A |
| 266 | A |
| 267 | A |
| 268 | A |
| 269 | A |
| 270 | A |
| 271 | A |
| 272 | A |
| 273 | A |
| 274 | A |
| 275 | A |
| 276 | C |
| 277 | A |
| 278 | A |
| 279 | A |
| 280 | A |
| 281 | B |
| 282 | A |
| 283 | A |
| 284 | A |
| 285 | B |
| 286 | A |
| 287 | A |
| 288 | E |
| 289 | B |
| 290 | A |
| 291 | A |
| 292 | A |
| 293 | B |
| 294 | A |
| 295 | A |
| 296 | B |
| 297 | A |
| 298 | C |
| 299 | C |
| 300 | C |
| 301 | A |
| 302 | A |
| 303 | A |
| 304 | A |
| 305 | A |
| 306 | A |
| 307 | A |
| 308 | A |
| 309 | A |
| 310 | A |
| 311 | A |
| 312 | B |
| 313 | A |
| 314 | C |
| 315 | A |
| 316 | A |
| 317 | A |
| 318 | A |
| 319 | A |
| 320 | A |
| 321 | A |
| 322 | A |
| 323 | A |
| 324 | A |
| 325 | A |
| 326 | A |
| 327 | A |
| 328 | B |
| 329 | A |
| 330 | A |
| 331 | A |
| 332 | A |
| 334 | A |
| 335 | A |
| 336 | A |
| 337 | A |
| 338 | A |
| 339 | A |
| 340 | A |
| 341 | A |
| 342 | A |
| 343 | A |
| 344 | A |
| 345 | A |
| 346 | B |
| 347 | A |
| 348 | A |
| 349 | A |
| 350 | A |
| 351 | B |
| 352 | A |
| 353 | A |
| 354 | A |
| 355 | A |
| 356 | A |
| 357 | A |
| 358 | A |
| 359 | A |
| 360 | A |
| 361 | A |
| 362 | A |
| 363 | A |
| 364 | A |
| 365 | A |
| 366 | A |
| 367 | A |
| 368 | A |
| 369 | A |
| 370 | A |
| 371 | A |
| 372 | A |
| 373 | A |
| 374 | A |
| 375 | A |
| 376 | A |
| 377 | A |
| 378 | A |
| 379 | A |
| 380 | A |
| 381 | A |
| 382 | A |
| 383 | A |
| 384 | A |
| 406 | A |
| 407 | A |
| 408 | A |
| 409 | A |

A: IC₅₀ ≤ 5 nM
B: 5 nM < IC₅₀ ≤ 10 nM
C: 10 nM < IC₅₀ ≤ 50 nM
D: 50 nM < IC₅₀ ≤ 100 nM
E: IC₅₀ > 100 nM

1. A compound of Formula (I), or pharmaceutically acceptable salt thereof: 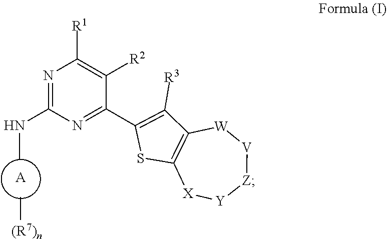
wherein
R¹ is hydrogen, halogen, C₁-C₆alkyl, C₁-C₆haloalkyl, C₁-C₆hydroxyalkyl, C₁-C₆aminoalkyl, or C₁-C₆heteroalkyl;
R² is hydrogen, halogen, C₁-C₆alkyl, C₁-C₆haloalkyl, C₁-C₆hydroxyalkyl, C₁-C₆aminoalkyl, C₁-C₆heteroalkyl, —C(═O)NR^{c}R^{d}, or cycloalkyl;
R³ is hydrogen, halogen, C₁-C₆alkyl, C₁-C₆haloalkyl, C₁-C₆hydroxyalkyl, C₁-C₆aminoalkyl, or C₁-C₆heteroalkyl;
W is —C(═O)—, —S(═O)—, —S(═O)₂—, —C(═O)NR⁴—, —S(═O)₂NR⁴—, —NR⁴S(═O)₂—, or —S(═O)(═NR⁴)—;
X is —NR⁵—, —O—, —S—, —S(═O)₂—, —C(R⁶)₂—, —C(═O)—, —C(═O)NR⁵—, or null;
Y is —C(R⁶)₂—, —O—, —NR⁵—, or null;
Z is —C(R⁶)₂—, —NR⁵—, or null;
or Y—Z is —CR⁶═CR⁶—, —CR⁶═N—, or —N═CR⁶—;
V is —C(R⁶)₂— or null;
wherein 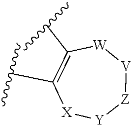
is a 5- to 8-membered ring;
R⁴ is hydrogen or C₁-C₆alkyl;
R⁵ is hydrogen, —C(═O)R^{a}, —C(═O)OR^{b}, —C(═O)NR^{c}R^{d}, —S(═O)₂NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl is optionally and independently substituted with one or more R;
each R⁶ is independently hydrogen, halogen, —CN, —NO₂, —OH, —OR^{a}, —OC(═O)NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R⁶ on the same atom are taken together to form an oxo;
Ring A is aryl or heteroaryl;
each R⁷ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl is optionally and independently substituted with one or more R^{7a};
or two R⁷ on the same atom are taken together to form an oxo;
or two R⁷ are taken together to form a heterocycloalkyl optionally substituted with one or more R^{7b};
each R^{7a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7a} on the same atom are taken together to form an oxo;
each R^{7b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7b} on the same atom are taken together to form an oxo;
n is 0-7;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
or two R on the same atom form an oxo.

2. The compound of claim 1, or pharmaceutically acceptable salt thereof, wherein W is —C(═O)NR⁴—.

3. The compound of claim 1, or pharmaceutically acceptable salt thereof, wherein W is —S(═O)₂NR⁴— or —NR⁴S(═O)₂—.

4. The compound of claim 1, or pharmaceutically acceptable salt thereof, wherein W is —S(═O)₂NR⁴—.

5. The compound of claim 1, or pharmaceutically acceptable salt thereof, wherein W is —NR⁴S(═O)₂—.

6. The compound of claim 1, or pharmaceutically acceptable salt thereof, wherein W is —C(═O)—.

7. The compound of claim 1, or pharmaceutically acceptable salt thereof, wherein W is —S(═O)₂—.

8. The compound of claim 1, or pharmaceutically acceptable salt thereof, wherein W is —S(═O)(═NR⁴)—.

9. The compound of any one of claims 1-8, or pharmaceutically acceptable salt thereof, wherein X is —NR⁵—.

10. The compound of any one of claims 1-8, or pharmaceutically acceptable salt thereof, wherein X is —O—.

11. The compound of any one of claims 1-8, or pharmaceutically acceptable salt thereof, wherein X is —S—.

12. The compound of any one of claims 1-8, or pharmaceutically acceptable salt thereof, wherein X is —C(═O)—.

13. The compound of any one of claims 1-8, or pharmaceutically acceptable salt thereof, wherein X is —C(═O)NR⁵—.

14. The compound of any one of claims 1-8, or pharmaceutically acceptable salt thereof, wherein X is —C(R⁶)₂—.

15. The compound of any one of claims 1-8, or pharmaceutically acceptable salt thereof, wherein X is null.

16. The compound of any one of claims 1-15, or pharmaceutically acceptable salt thereof, wherein Y is —C(R⁶)₂—.

17. The compound of any one of claims 1-15, or pharmaceutically acceptable salt thereof, wherein Y is null.

18. The compound of any one of claims 1-15, or pharmaceutically acceptable salt thereof, wherein Y is —NR⁵—.

19. The compound of any one of claims 1-15, or pharmaceutically acceptable salt thereof, wherein Y is —O—.

20. The compound of any one of claims 1-19, or pharmaceutically acceptable salt thereof, wherein Z is —C(R⁶)₂—.

21. The compound of any one of claims 1-19, or pharmaceutically acceptable salt thereof, wherein Z is null.

22. The compound of any one of claims 1-15, or pharmaceutically acceptable salt thereof, wherein Y—Z is —CR⁶═CR⁶—.

23. The compound of any one of claims 1-15, or pharmaceutically acceptable salt thereof, wherein Y—Z is —CR⁶═N—.

24. The compound of any one of claims 1-15, or pharmaceutically acceptable salt thereof, wherein Y—Z is —N═CR⁶—.

25. The compound of any one of claims 1-24, or pharmaceutically acceptable salt thereof, wherein V is —C(R⁶)₂—.

26. The compound of any one of claims 1-24, or pharmaceutically acceptable salt thereof, wherein V is null.

27. The compound of claim 1, or pharmaceutically acceptable salt thereof, wherein

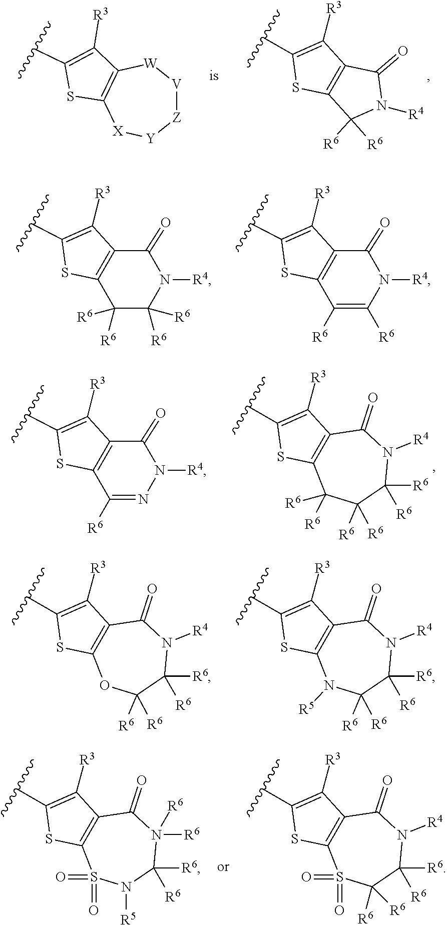

28. The compound of claim 1, or pharmaceutically acceptable salt thereof, wherein

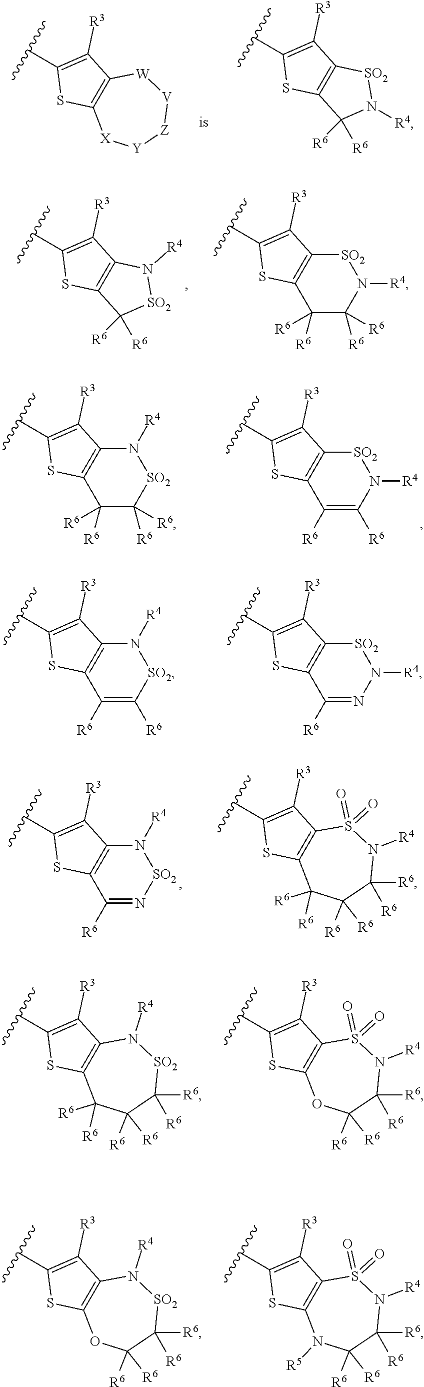

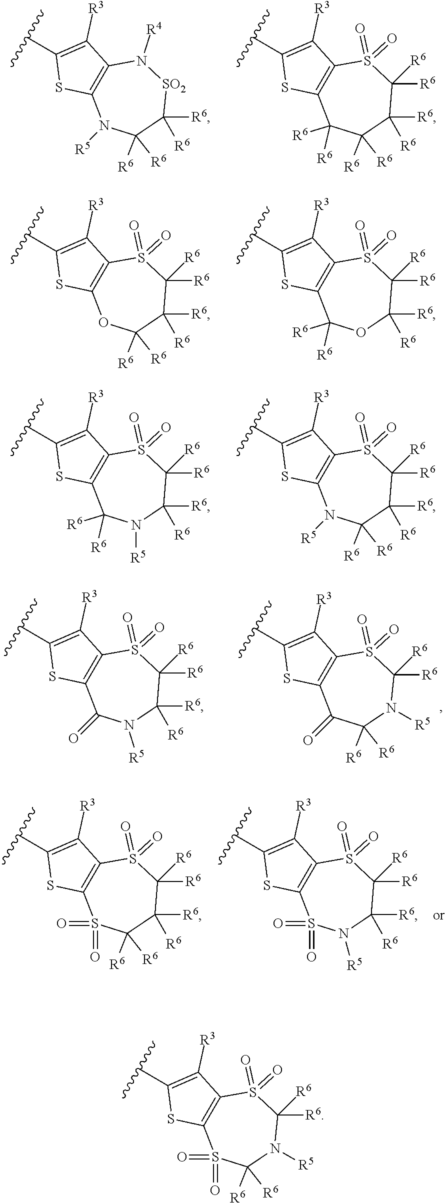

29. The compound of claim 1, or pharmaceutically acceptable salt thereof, wherein

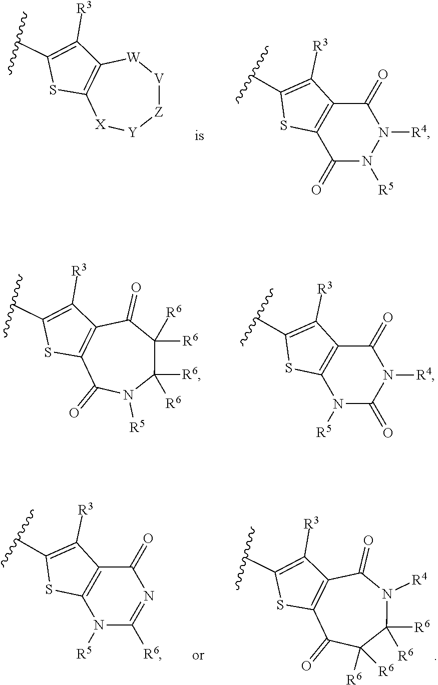

30. The compound of any one of claims 1-29, or pharmaceutically acceptable salt thereof, wherein R⁴ is hydrogen.

31. The compound of any one of claims 1-29, or pharmaceutically acceptable salt thereof, wherein R⁴ is C₁-C₆alkyl.

32. The compound of any one of claims 1-31, or pharmaceutically acceptable salt thereof, wherein R⁵ is hydrogen.

33. The compound of any one of claims 1-31, or pharmaceutically acceptable salt thereof, wherein R⁵ is C₁-C₆alkyl.

34. The compound of any one of claims 1-33, or pharmaceutically acceptable salt thereof, wherein each R⁶ is independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally and independently substituted with one or more R; or two R⁶ on the same atom are taken together to form an oxo.

35. The compound of any one of claims 1-34, or pharmaceutically acceptable salt thereof, wherein each R⁶ is independently hydrogen, halogen, C₁-C₆alkyl, or C₁-C₆haloalkyl; or two R⁶ on the same atom are taken together to form an oxo.

36. The compound of any one of claims 1-35, or pharmaceutically acceptable salt thereof, wherein each R⁶ is independently hydrogen or C₁-C₆alkyl; or two R⁶ on the same atom are taken together to form an oxo.

37. The compound of any one of claims 1-36, or pharmaceutically acceptable salt thereof, wherein each R⁶ is hydrogen.

38. A compound of Formula (II), or pharmaceutically acceptable salt thereof: 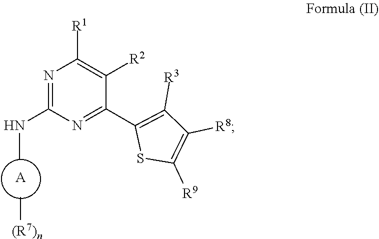
wherein
R¹ is hydrogen, halogen, C₁-C₆alkyl, C₁-C₆haloalkyl, C₁-C₆hydroxyalkyl, C₁-C₆aminoalkyl, or C₁-C₆heteroalkyl;
R² is halogen, C₁-C₆alkyl, C₁-C₆haloalkyl, C₁-C₆hydroxyalkyl, C₁-C₆aminoalkyl, C₁-C₆heteroalkyl, —C(═O)NR^{c}R^{d}, or cycloalkyl;
R³ is hydrogen, halogen, C₁-C₆alkyl, C₁-C₆haloalkyl, C₁-C₆hydroxyalkyl, C₁-C₆aminoalkyl, or C₁-C₆heteroalkyl;
R⁸ is —CN, —NR^{c}R^{d}, —C(═O)NR^{c}R^{d}, —C(═O)R^{a}, —S(═O)R^{a}, —S(═O)₂R^{a}, —S(═O)(═NR^{b})R^{a}, —S(═O)₂NR^{c}R^{d}, —NR^{b}S(═O)₂R^{a}, —P(═O)(R^{a})₂, heterocycloalkyl, or heteroaryl; wherein the heterocycloalkyl and heteroaryl is independently optionally substituted with one or more R;
R⁹ is hydrogen, halogen, —C(═O)NR^{c}R^{d}, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkyl(cycloalkyl), or C₁-C₆alkyl(heterocycloalkyl); wherein the alkyl, cycloalkyl, and heterocycloalkyl is optionally and independently substituted with one or more R;
Ring A is aryl or heteroaryl;
each R⁷ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein the alkyl, alkenyl, alkynyl, cycloalkyl, heterocycloalkyl, aryl, and heteroaryl is optionally and independently substituted with one or more R^{7a};
or two R⁷ on the same atom are taken together to form an oxo;
or two R⁷ are taken together to form a heterocycloalkyl optionally substituted with one or more R^{7b};
each R^{7a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7a} on the same atom are taken together to form an oxo;
each R^{7b} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{7b} on the same atom are taken together to form an oxo;
n is 0-7;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
or two R on the same atom form an oxo;
provided that the compound is not

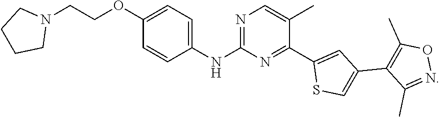 [SMILES: Cc1cnc(Nc2ccc(OCCN3CCCC3)cc2)nc1-c1cc(-c2c(C)noc2C)cs1]

39. The compound of claim 38, or pharmaceutically acceptable salt thereof, wherein R⁸ is —CN, —NR^{c}R^{d}, —C(═O)NR^{c}R^{d}, —S(═O)₂R^{a}, —S(═O)₂NR^{c}R^{d}, —NR^{b}S(═O)₂R^{a}, or —P(═O)(R^{a})₂.

40. The compound of claim 38, or pharmaceutically acceptable salt thereof, wherein R⁸ is —C(═O)NR^{c}R^{d}, —S(═O)₂R^{a}, —S(═O)₂NR^{c}R^{d}, —NR^{b}S(═O)₂R^{a}, or —P(═O)(R^{a})₂.

41. The compound of claim 38, or pharmaceutically acceptable salt thereof, wherein R⁸ is —C(═O)NR^{c}R^{d}.

42. The compound of claim 38, or pharmaceutically acceptable salt thereof, wherein R⁸ is —P(═O)(R^{a})₂.

43. The compound of claim 38, or pharmaceutically acceptable salt thereof, wherein R⁸ is —C(═O)R^{a}.

44. The compound of claim 38, or pharmaceutically acceptable salt thereof, wherein R⁸ is —S(═O)R^{a}.

45. The compound of claim 38, or pharmaceutically acceptable salt thereof, wherein R⁸ is —S(═O)(═NR^{b})R^{a}.

46. The compound of claim 38, or pharmaceutically acceptable salt thereof, wherein R⁸ is heterocycloalkyl or heteroaryl; wherein the heterocycloalkyl and heteroaryl is independently optionally substituted with one or more R.

47. The compound of claim 38, or pharmaceutically acceptable salt thereof, wherein R⁸ is heterocycloalkyl optionally substituted with one or more R.

48. The compound of any one of claims 38-47, or pharmaceutically acceptable salt thereof, wherein R⁹ is hydrogen, halogen, C₁-C₆alkyl, or C₁-C₆haloalkyl.

49. The compound of any one of claims 38-48, or pharmaceutically acceptable salt thereof, wherein R⁹ is hydrogen or C₁-C₆alkyl.

50. The compound of any one of claims 38-49, or pharmaceutically acceptable salt thereof, wherein R⁹ is hydrogen.

51. The compound of any one of claims 1-50, or pharmaceutically acceptable salt thereof, wherein R¹ is hydrogen, halogen, C₁-C₆alkyl, or C₁-C₆haloalkyl.

52. The compound of any one of claims 1-51, or pharmaceutically acceptable salt thereof, wherein R¹ is hydrogen or C₁-C₂alkyl.

53. The compound of any one of claims 1-52, or pharmaceutically acceptable salt thereof, wherein R¹ is hydrogen.

54. The compound of any one of claims 1-53, or pharmaceutically acceptable salt thereof, wherein R² is hydrogen, halogen, C₁-C₆alkyl, C₁-C₆haloalkyl, —C(═O)NR^{c}R^{d}, or cycloalkyl.

55. The compound of any one of claims 1-54, or pharmaceutically acceptable salt thereof, wherein R² is C₁-C₆alkyl, C₁-C₆haloalkyl, —C(═O)NR^{c}R^{d}, or cycloalkyl.

56. The compound of any one of claims 1-55, or pharmaceutically acceptable salt thereof, wherein R² is C₁-C₂alkyl, C₁-C₂haloalkyl, —C(═O)NR^{c}R^{d}, or cycloalkyl.

57. The compound of any one of claims 1-56, or pharmaceutically acceptable salt thereof, wherein R² is CF₃.

58. The compound of any one of claims 1-56, or pharmaceutically acceptable salt thereof, wherein R² is CH₃.

59. The compound of any one of claims 1-56, or pharmaceutically acceptable salt thereof, wherein R² is cyclopropyl.

60. The compound of any one of claims 1-56, or pharmaceutically acceptable salt thereof, wherein R² is —C(═O)NH₂.

61. The compound of any one of claims 1-60, or pharmaceutically acceptable salt thereof, wherein R³ is hydrogen, halogen, C₁-C₆alkyl, or C₁-C₆haloalkyl.

62. The compound of any one of claims 1-61, or pharmaceutically acceptable salt thereof, wherein R³ is hydrogen.

63. The compound of any one of claims 1-62, or pharmaceutically acceptable salt thereof, wherein Ring A is heteroaryl.

64. The compound of any one of claims 1-63, or pharmaceutically acceptable salt thereof, wherein Ring A is 5- or 6-membered heteroaryl.

65. The compound of any one of claims 1-64, or pharmaceutically acceptable salt thereof, wherein Ring A is 5-membered heteroaryl.

66. The compound of any one of claims 1-65, or pharmaceutically acceptable salt thereof, wherein Ring A is pyrazolyl.

67. The compound of any one of claims 1-62, or pharmaceutically acceptable salt thereof, wherein Ring A is phenyl.

68. The compound of any one of claims 1-67, or pharmaceutically acceptable salt thereof, wherein each R⁷ is independently halogen, —CN, —OH, —OR^{a}, —SR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally and independently substituted with one or more R^{7a}; or two R⁷ are taken together to form a heterocycloalkyl optionally substituted with one or more R^{7b}.

69. The compound of any one of claims 1-68, or pharmaceutically acceptable salt thereof, wherein each R⁷ is independently halogen, —CN, —OH, —OR^{a}, —SR^{a}, —NR^{c}R^{d}, C₁-C₆alkyl, C₁-C₆haloalkyl, cycloalkyl, or heterocycloalkyl; wherein the alkyl, cycloalkyl, and heterocycloalkyl is optionally and independently substituted with one or more R^{7a}; or two R⁷ are taken together to form a heterocycloalkyl optionally substituted with one or more R^{7b}.

70. The compound of any one of claims 1-69, or pharmaceutically acceptable salt thereof, wherein each R⁷ is independently halogen, —OR^{a}, —SR^{a}, C₁-C₆alkyl, cycloalkyl, or heterocycloalkyl; wherein the alkyl, cycloalkyl, and heterocycloalkyl is optionally and independently substituted with one or more R^{7a}; or two R⁷ are taken together to form a heterocycloalkyl optionally substituted with one or more R^{7b}.

71. The compound of any one of claims 1-70, or pharmaceutically acceptable salt thereof, wherein each R^{7a} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally and independently substituted with one or more R.

72. The compound of any one of claims 1-71, or pharmaceutically acceptable salt thereof, wherein each R^{7a} is independently halogen, C₁-C₆alkyl, or C₁-C₆haloalkyl.

73. The compound of any one of claims 1-72, or pharmaceutically acceptable salt thereof, wherein each R^{7a} is independently C₁-C₆alkyl.

74. The compound of any one of claims 1-73, or pharmaceutically acceptable salt thereof, wherein each R^{7b} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl, is optionally and independently substituted with one or more R.

75. The compound of any one of claims 1-74, or pharmaceutically acceptable salt thereof, wherein each R^{7b} is independently halogen, C₁-C₆alkyl, C₁-C₆haloalkyl, cycloalkyl, or heterocycloalkyl.

76. The compound of any one of claims 1-75, or pharmaceutically acceptable salt thereof, wherein n is 1-4.

77. The compound of any one of claims 1-76, or pharmaceutically acceptable salt thereof, wherein n is 1-3.

78. The compound of any one of claims 1-77, or pharmaceutically acceptable salt thereof, wherein n is 1 or 2.

79. The compound of any one of claims 1-62, or pharmaceutically acceptable salt thereof, wherein 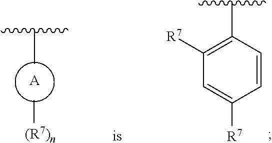
wherein:
each R⁷ is independently halogen, —OR^{a}, —SR^{a}, C₁-C₆alkyl, cycloalkyl, or heterocycloalkyl; wherein the cycloalkyl and heterocycloalkyl is optionally and independently substituted with one or more C₁-C₆alkyl.

80. The compound of any one of claims 1-62, or pharmaceutically acceptable salt thereof, wherein 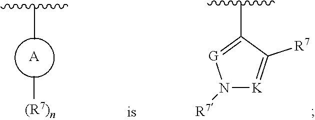
wherein:
G is —CH— and K is —N— or G is —N— and K is —CH—;
R^{7′} is cycloalkyl or heterocycloalkyl; wherein the cycloalkyl and heterocycloalkyl is optionally and independently substituted with one or more C₁-C₆alkyl; and
R⁷ is independently C₁-C₆alkyl, cycloalkyl, or heterocycloalkyl.

81. The compound of any one of claims 1-62, or pharmaceutically acceptable salt thereof, wherein 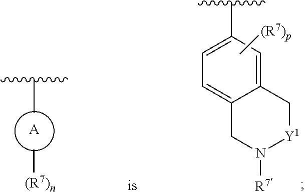
wherein:
Y¹ is —CH₂—, —C(═O)—, or null;
R^{7′} is hydrogen or C₁-C₆alkyl;
p is 0-5; and
each R⁷ is independently halogen, —OR^{a}, —SR^{a}, C₁-C₆alkyl, cycloalkyl, or heterocycloalkyl; or two R⁷ on the same atom are taken together to form an oxo.

82. The compound of any one of claims 1-62, or pharmaceutically acceptable salt thereof, wherein 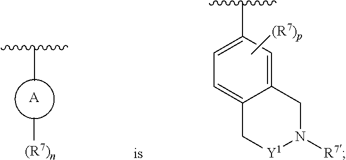
wherein:
Y¹ is —CH₂—, —C(═O)—, or null;
R^{7′} is hydrogen or C₁-C₆alkyl;
p is 0-5; and
each R⁷ is independently halogen, —OR^{a}, —SR^{a}, C₁-C₆alkyl, cycloalkyl, or heterocycloalkyl; or two R⁷ on the same atom are taken together to form an oxo.

83. The compound of any one of claims 1-62, or pharmaceutically acceptable salt thereof, wherein 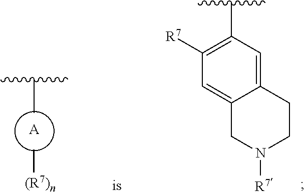
wherein:
R^{7′} is hydrogen or C₁-C₆alkyl; and
R⁷ is independently halogen, —OR^{a}, —SR^{a}, C₁-C₆alkyl, cycloalkyl, or heterocycloalkyl.

84. The compound of any one of claims 1-62, or pharmaceutically acceptable salt thereof, wherein 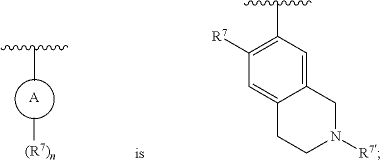
wherein:
R^{7′} is hydrogen or C₁-C₆alkyl; and
R⁷ is independently halogen, —OR^{a}, —SR^{a}, C₁-C₆alkyl, cycloalkyl, or heterocycloalkyl.

85. The compound of any one of claims 1-62, or pharmaceutically acceptable salt thereof, wherein 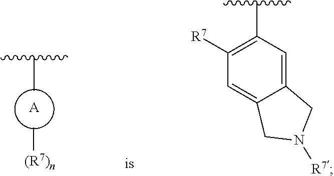
wherein:
R^{7′} is hydrogen or C₁-C₆alkyl; and
R⁷ is independently halogen, —OR^{a}, —SR^{a}, C₁-C₆alkyl, cycloalkyl, or heterocycloalkyl.

86. The compound of any one of claims 1-62, or pharmaceutically acceptable salt thereof, wherein 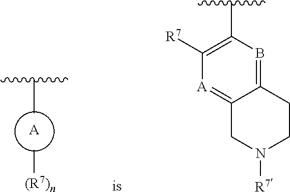
wherein:
A and B are independently selected from CH, N, and CF, with the proviso that at least one of A or B is N or CF.
R^{7′} is hydrogen or C₁-C₆alkyl; and
R⁷ is independently halogen, —OR^{a}, —SR^{a}, C₁-C₆alkyl, cycloalkyl, or heterocycloalkyl.

87. The compound of any one of claims 1-62, or pharmaceutically acceptable salt thereof, wherein 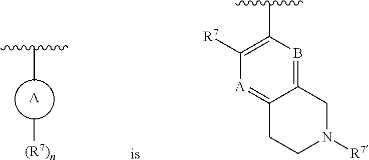
wherein:
A and B are independently selected from CH, N, and CF, with the proviso that at least one of A or B is N or CF.
R^{7′} is hydrogen or C₁-C₆alkyl; and
R⁷ is independently halogen, —OR^{a}, —SR^{a}, C₁-C₆alkyl, cycloalkyl, or heterocycloalkyl.

88. The compound of any one of claims 1-62, or pharmaceutically acceptable salt thereof, wherein

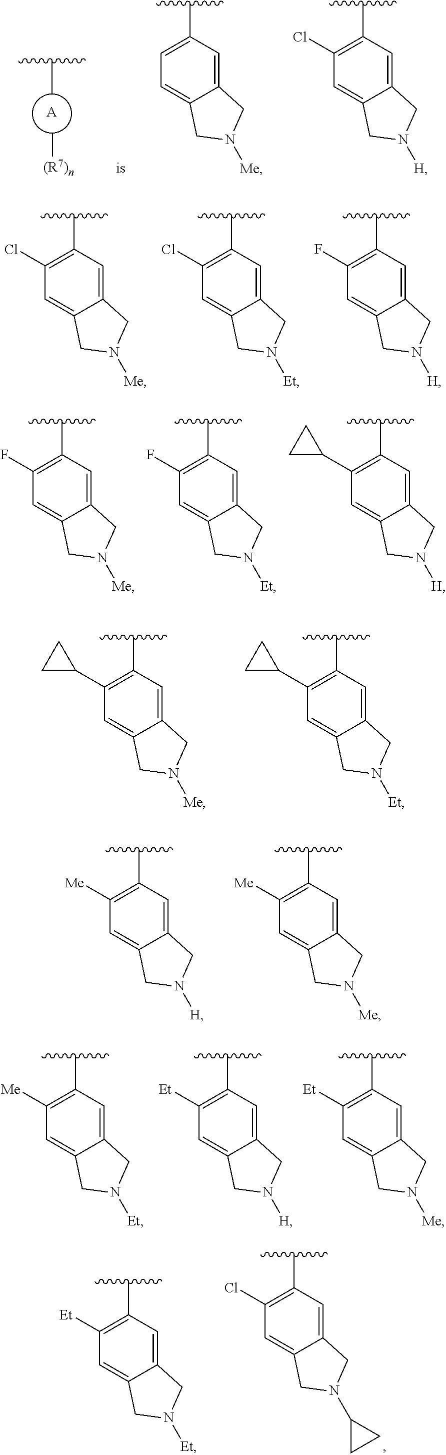

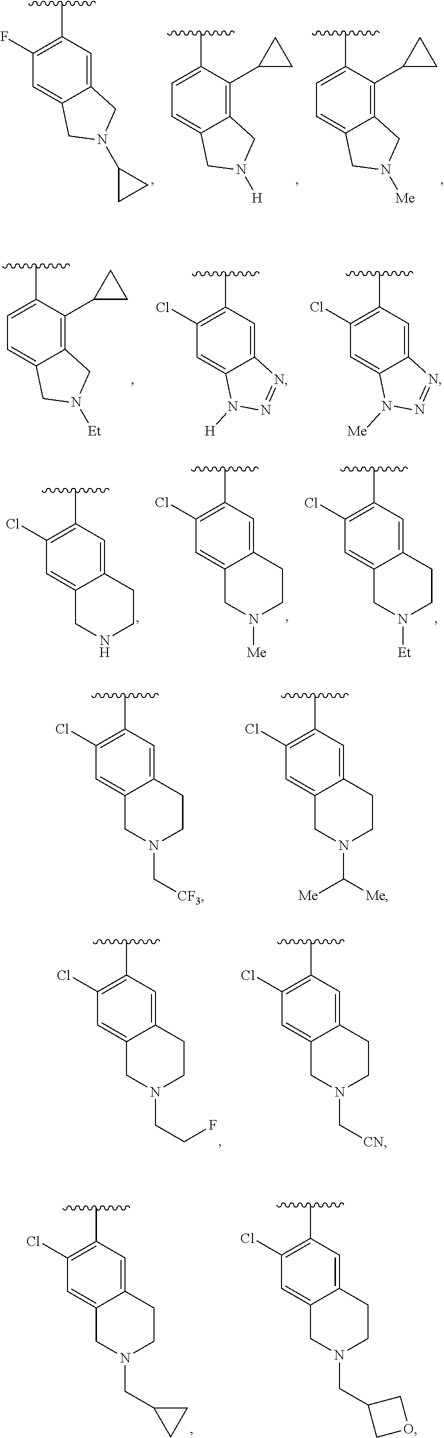

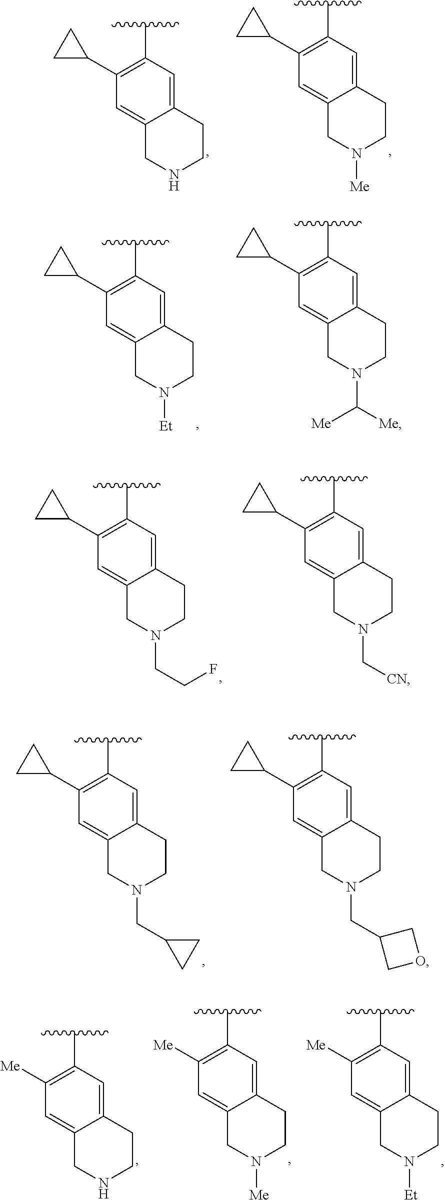

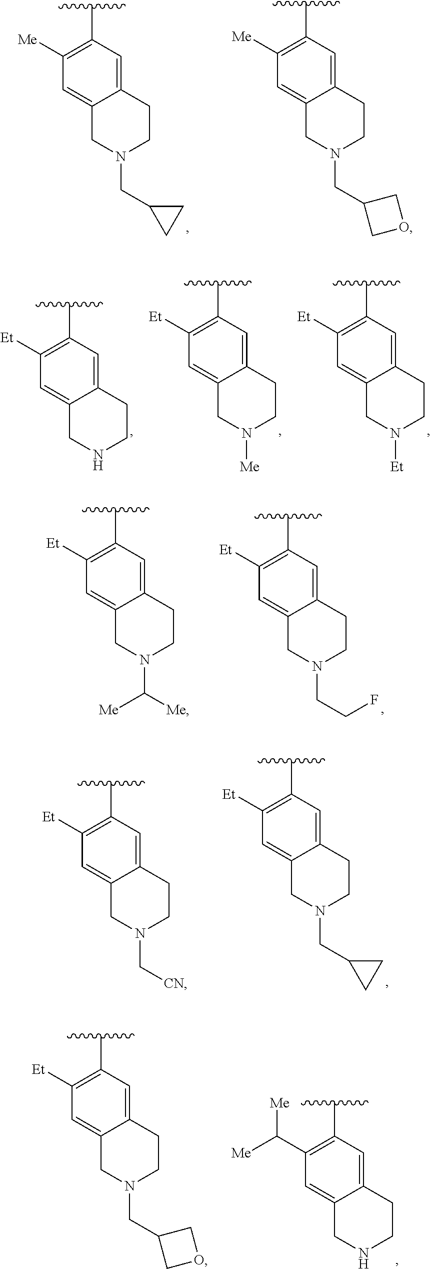

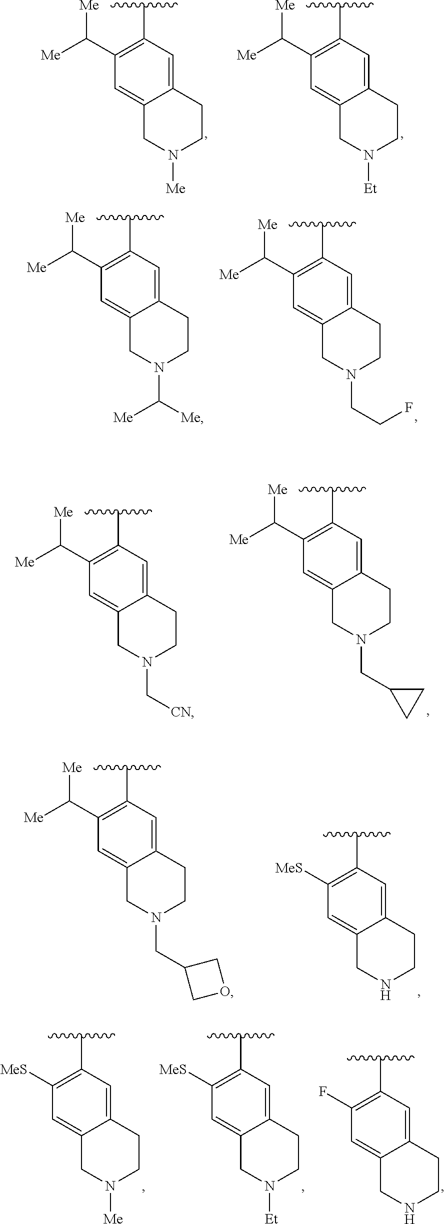

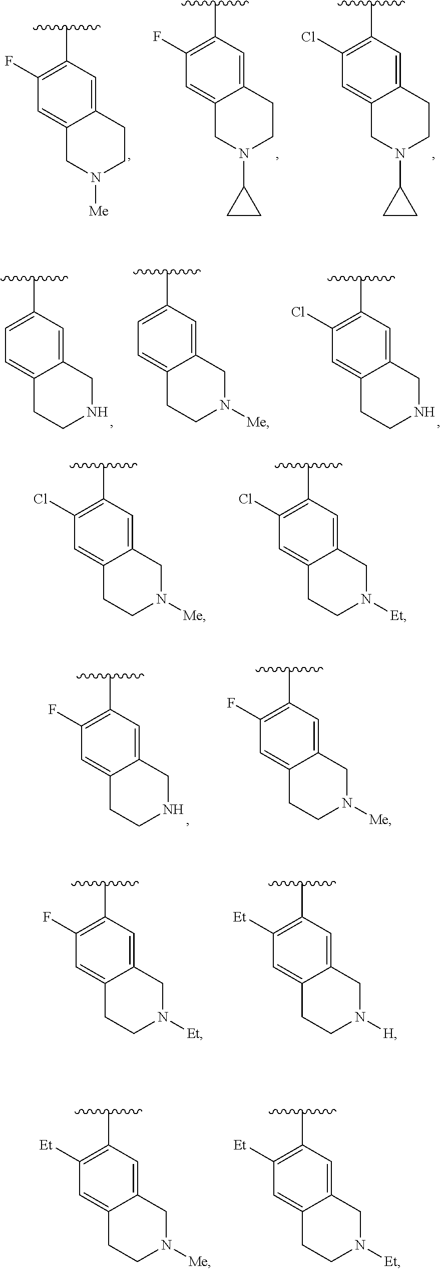

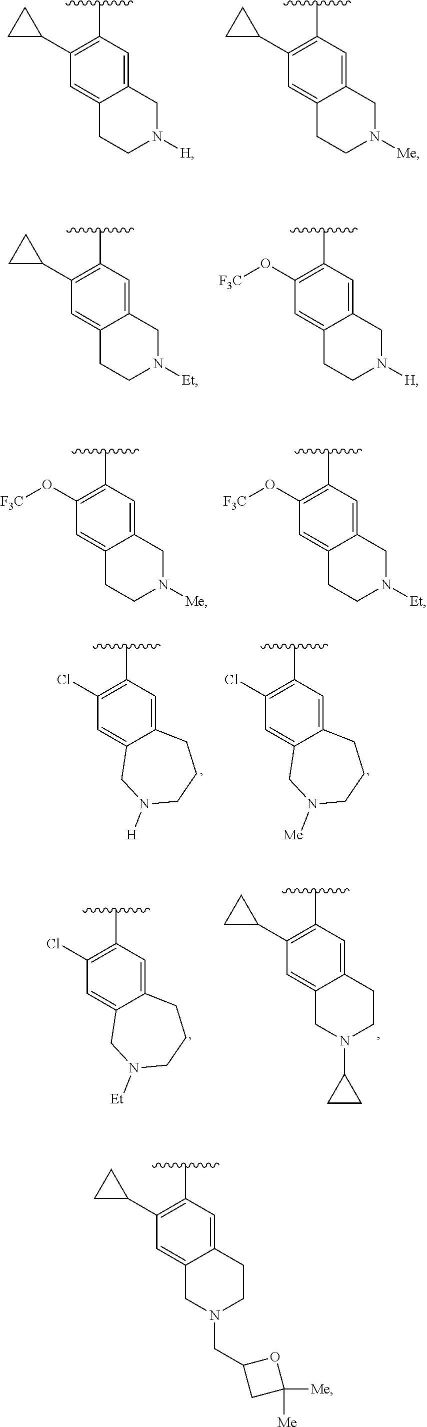

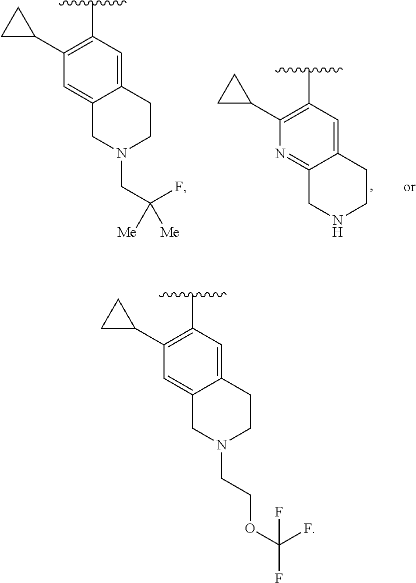 [SMILES: *c1cc2c(cc1C1CC1)CN(CC(C)(C)F)CC2 ; *c1cc2c(nc1C1CC1)CNCC2 ; *c1cc2c(cc1C1CC1)CN(CCOC(F)(F)F)CC2]

89. The compound of any one of claims 1-62, or pharmaceutically acceptable salt thereof, wherein

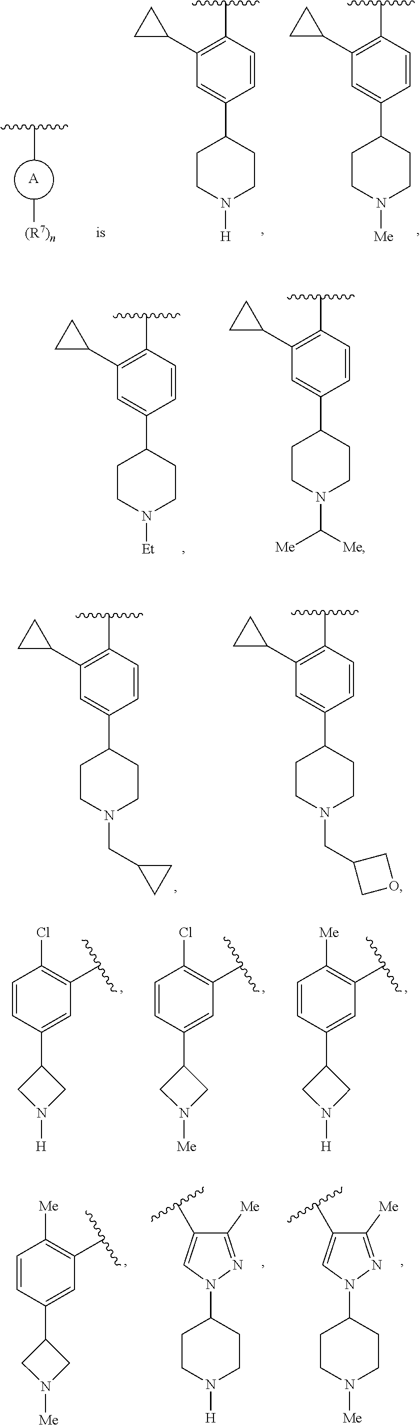

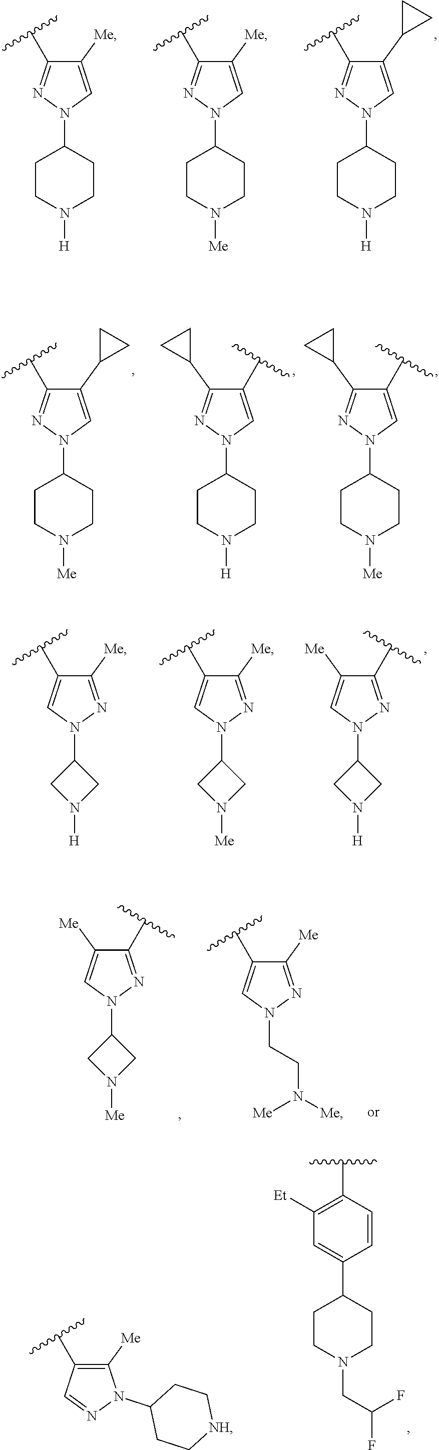

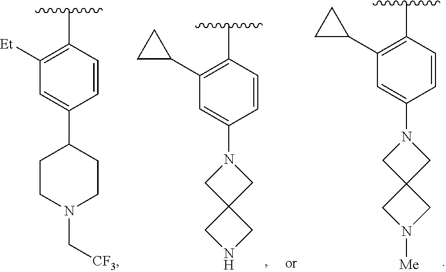

90. A compound that is selected from a compound disclosed in the specification, or pharmaceutically acceptable salt thereof.

91. A pharmaceutical composition comprising a compound of any one of claims 1-90, or pharmaceutically acceptable salt thereof, and a pharmaceutically acceptable excipient.

92. A method of inhibiting a Unc-51 like autophagy activating kinase (ULK) isoform comprising contacting the ULK isoform with a compound of any one of claims 1-90, or pharmaceutically acceptable salt thereof, or pharmaceutical composition of claim 91.

93. The method of claim 88, that inhibits ULK1 and ULK2.

94. A method of treating cancer comprising administering to a subject a compound of any one of claims 1-90, or pharmaceutically acceptable salt thereof, or pharmaceutical composition of claim 91.

95. Use of a compound or pharmaceutically acceptable salt of any one of claims 1-90, or pharmaceutically acceptable salt thereof, in the manufacture of a medicament for the treatment of cancer.
